(19) United States
(12) Patent Application Publication
(10) Pub. No.: US 2023/0272429 A1
(43) Pub. Date: Aug. 31, 2023

(54) MODIFICATION OF BLOOD TYPE ANTIGENS

(71) Applicant: Sana Biotechnology, Inc., Seattle, WA (US)

(72) Inventors: William DOWDLE, South San Francisco, CA (US); Andrew MAY, San Francisco, CA (US); Sonja SCHREPFER, South San Francisco, CA (US)

(21) Appl. No.: 17/792,121

(22) PCT Filed: Jan. 12, 2021
(86) PCT No.: PCT/US2021/013140

§ 371 Date: Jul. 11, 2022

Related U.S. Application Data
(60) Provisional application No. 62/960,617, filed on Jan. 13, 2020.
(60) Provisional application No. 62/960,607, filed on Jan. 13, 2020.

(51) Int. Cl. C12N 15/90; C12N 9/22; C12N 15/11; A61K 35/545

(52) U.S. Cl. CPC C12N 15/907; C12N 9/22; C12N 15/11; C12N 15/111; A61K 35/545; C12N 2310/20; C12N 2800/80

(57) ABSTRACT

Provided herein are cells with a gene modification of an ABO gene, RHD gene, and/or FUT1 gene. In some embodiments, the cells express reduced levels of a MHC I antigen and/or a MHC II antigen. In some instances, the cells are also hypoimmunogenic cells.

### CROSS-REFERENCE

This application claims priority as a 371 of International Application No. PCT/US21/13140, filed Jan. 12, 2021, which claims priority under 35 U.S.C. § 119(e) to U.S. Provisional Application No. 62/960,607, filed Jan. 13, 2020, and U.S. Provisional Application No. 62/960,617, filed Jan. 13, 2020, the disclosures of which are herein incorporated by reference in their entirety.

### BACKGROUND OF THE INVENTION

Blood products can be classified into different groups according to the presence or absence of antigens on the surface of every red blood cell in a person's body (ABO Blood Type). The A, B, AB, and A1 antigens are determined by the sequence of oligosaccharides on the glycoproteins of erythrocytes. The genes in the blood group antigen group provide instructions for making antigen proteins. Blood group antigen proteins serve a variety of functions within the cell membrane of red blood cells. These protein functions include transporting other proteins and molecules into and out of the cell, maintaining cell structure, attaching to other cells and molecules, and participating in chemical reactions.

The Rhesus Factor (Rh) blood group is the second most important blood group system, after the ABO blood group system. The Rh blood group system consists of 49 defined blood group antigens, among which five antigens, D, C, c, E, and e, are the most important. Rh(D) status of an individual is normally described with a positive or negative suffix after the ABO type. The terms “Rh factor,” “Rh positive,” and “Rh negative” refer to the Rh(D) antigen only. Antibodies to Rh antigens can be involved in hemolytic transfusion reactions and antibodies to the Rh(D) and Rh(c) antigens confer significant risk of hemolytic disease of the fetus and newborn. ABO antibodies develop in early life in every human. However, rhesus antibodies in Rh− humans typically develop only when the person is sensitized. This can occur, for example, by giving birth to a Rh+ baby or by receiving an Rh+ blood transfusion.

A, B, H, and Rh antigens are major determinants of histocompatibility between donor and recipient for blood, tissue and cellular transplantation. A glycosyltransferase activity encoded by the ABO gene is responsible for producing A, B, AB, O histo-blood group antigens, which are displayed on the surface of cells. Group A individuals encode an ABO gene product with specificity to produce α(1,3) N-acetylgalactosaminyltransferase activity and group B individuals with specificity to produce α(1, 3) galactosyltransferase activity. Type O individuals do not produce a functional galactosyltransferase at all and thus do not produce either modification. Type AB individuals harbor one copy of each and produce both types of modifications. The enzyme products of the ABO gene act on the H antigen as a substrate, and thus type O individuals whom lack ABO activity present an unmodified H antigen and are thus often referred to as type O(H).

The H antigen itself is the product of an α(1,2)fucosyltransferase enzyme, which is encoded by the FUT1 gene. In very rare individuals there exists a loss of the H antigen entirely as a result of a disruption of the FUT1 gene and no substrate will exist for ABO to produce A or B histo-blood types. These individuals are said to be of the Bombay histo-blood type. The Rh antigen is encoded by the RHD gene, and individuals who are Rh negative harbor a deletion or disruption of the RHD gene.

The availability of cell-lines suitable for therapeutic applications is severely limited and often the available cell lines are not universally histo-compatible with all possible recipients.

There remains a need for novel approaches, compositions and methods for generating histo-blood type cells that are useful for cell therapies.

### BRIEF SUMMARY OF THE INVENTION

In some aspects, provided herein is an isolated cell in which expression of an ABO gene is partially or fully inactivated by a deleterious variation of the ABO gene or by insertion of an exon 6 258delG variation of the ABO gene, and/or expression of an RHD gene is partially or fully inactivated by a deleterious variation of the RHD gene.

In some embodiments, expression of the ABO gene is partially or fully inactivated by an insertion or deletion within ACACCT of exon 1; AGTGCG of exon 2; or CAAGCGC, GGCGCA, TGGGCG, ACCACG, GCCGCA, CCACGT, TGCCGT, TACTCG, ACTCGG, GAGCGC, GCCTGG, GTCCTT, TCACGC, TGGACG, TGGTCG, GCTGGC, CACGCG, AGGTGA, or GCATCG of exon 8 of the ABO gene.

In some embodiments, the deleterious variation of the ABO gene is generated using CRISPR-Cas9 gene editing comprising a guide RNA target sequence comprising one selected from the group consisting of those in Tables A-C.

In some embodiments, the deleterious variation of the ABO gene comprises an insertion/deletion in a coding exon and the guide RNA target sequence comprises SEQ ID NO:1 or SEQ ID NO:2.

In some embodiments, the deleterious variation of the ABO gene comprises a frame-shift insertion/deletion and the guide RNA target sequence comprises SEQ ID NO:3.

In some embodiments, the deleterious variation of the ABO gene is a homozygous variation such that the cell is a histo-blood type O cell.

In some embodiments, the exon 6 258delG variation (e.g., the deletion in exon 6 of G at nucleotide position 258) of the ABO gene is generated using CRISPR-Cas9 gene editing comprising a guide RNA target sequence comprising one selected from the group consisting of SEQ ID NOS:3-5 and a homology directed repair (HDR) template comprising a sequence encoding the 258delG variation, a PAM sequence, a spacer region, and two homology arms, each comprising about 10 nucleotides to about 1 Kb of homologous sequence to the ABO gene.

In some embodiments, the HDR template further comprises one or more silent mutations in the PAM sequence and/or the spacer region to ensure that the HDR template is not cleaved by Cas9.

In some embodiments, the exon 6 258delG variation of the ABO gene is generated using CRISPR-Cas9 gene editing comprising a guide RNA target sequence comprising one selected from the group consisting of SEQ ID NOS:3-5 and the HDR template comprises one selected from the group consisting of SEQ ID NOS:6-8.

In some embodiments, the exon 6 258delG variation of the ABO gene is a homozygous variation such that the cell is a histo-blood type O cell.

In some embodiments, the deleterious variation of the RHD gene is generated by using CRISPR-Cas9 gene editing comprising one or more guide RNA target sequences comprising one selected from the group consisting of those in Tables D-F.

In some embodiments, the deleterious variation comprises a deletion of a genomic region comprising exons 1-8 of the RHD gene and is generated by using CRISPR-Cas9 gene editing comprising a guide RNA target sequence comprising SEQ ID NO:11 and another guide RNA comprising SEQ ID NO:12.

In some embodiments, the deleterious variation of the RHD gene comprises a frame-shift insertion/deletion and the guide RNA target sequence comprises SEQ ID NO:9 or SEQ ID NO:10.

In some embodiments, expression of the RHD gene is partially or fully inactivated by an insertion or deletion within TCATGG, GAGGTG, AACTCG, AGTTTC, TTGGCT, or CACAGC of exon 2; CCGTGA of exon 3; GGGTAG or AGGGAA of exon 4; TTCGAT, TCAGCG, CATAGT, or ATCGAA of exon 5; CGTCGG or TCCGTC of exon 6; CGGCAA, CGGAGC, TACCGT, GCTTGC, or CTTGCT of exon 7; or GGTTCT or TCCTAC of exon 8 of the RHD gene.

In some embodiments, the deleterious variation of the RHD gene is a homozygous variation such that the cell is a Rhesus factor (Rh) negative cell. In some embodiments, the deleterious variation of the RHD gene is a homozygous variation such that the cell is a type O negative cell.

In some aspects, provided herein is a cell in which expression of an ABO gene is partially or fully inactivated by a deleterious variation of the ABO gene or by insertion of an exon 6 258delG variation of the ABO gene.

In some embodiments, the cell is an Rh negative cell. In some embodiments, the deleterious variation of the ABO gene is generated using CRISPR-Cas9 gene editing comprising a guide RNA target sequence comprising one selected from the group consisting of those in Tables A-C.

In some embodiments, the deleterious variation of the ABO gene comprises an insertion/deletion in a coding exon and a guide RNA target sequence comprises SEQ ID NO:1 or SEQ ID NO:2.

In some embodiments, the deleterious variation of the ABO gene comprises a frame-shift insertion/deletion and the guide RNA target sequence comprises SEQ ID NO:3.

In some embodiments, expression of the ABO gene is partially or fully inactivated by an insertion or deletion within ACACCT of exon 1; AGTGCG of exon 2; or CAAGCGC, GGCGCA, TGGGCG, ACCACG, GCCGCA, CCACGT, TGCCGT, TACTCG, ACTCGG, GAGCGC, GCCTGG, GTCCTT, TCACGC, TGGACG, TGGTCG, GCTGGC, CACGCG, AGGTGA, or GCATCG of exon 8 of the ABO gene.

In some embodiments, the deleterious variation of the ABO gene is a homozygous variation such that the cell is a type O negative cell.

In some embodiments, the exon 6 258delG variation of the ABO gene is generated using CRISPR-Cas9 gene editing comprising a guide RNA target sequence comprising one selected from the group consisting of those in SEQ ID NOS:3-5 and a HDR template comprising a sequence encoding the 258delG variation, a PAM sequence, a spacer region, and two homology arms, each comprising about 10 nucleotides to about 1 Kb of homologous sequence to the ABO gene.

In some embodiments, the HDR template further comprises one or more silent mutations in the PAM sequence and/or the spacer region to ensure that the HDR template is not cleaved by Cas9.

In some embodiments, the exon 6 258delG variation of the ABO gene is generated using CRISPR-Cas9 gene editing comprising a guide RNA target sequence comprises one selected from the group consisting of SEQ ID NOS:3-5 and the HDR template comprises one selected from the group consisting of SEQ ID NOS:6-8.

In some embodiments, the exon 6 258delG variation of the ABO gene is a homozygous variation such that the cell is a type O negative cell.

In some aspects, provided herein is an isolated cell in which expression of a FUT1 gene is partially or fully inactivated by a deleterious variation of the FUT1 gene and/or expression of an RHD gene is partially or fully inactivated by a deleterious variation of the RHD gene.

In some embodiments, expression of the FUT1 gene is partially or fully in isolated activated by an insertion or deletion within ATCGAC, AGTACG, CGCCCT, GTTTGC, CGACAA, GGTGCG, CGCCGT, ACGCCG, CCGGTT, CGCGGG, TTTTCG, ATACCG, GTGCGC, CCATTG, TGTCGG, ATCTGC, CTTTGT, GGGGCC, GGCCAT, TGCGAT, CGTGCA, or ATGGAC of exon 4 of the FUT1 gene.

In some embodiments, expression of the RHD gene is partially or fully inactivated by an insertion or deletion within TCATGG, GAGGTG, AACTCG, AGTTTC, TTGGCT, or CACAGC of exon 2; CCGTGA of exon 3; GGGTAG or AGGGAA of exon 4; TTCGAT, TCAGCG, CATAGT, or ATCGAA of exon 5; CGTCGG or TCCGTC of exon 6; CGGCAA, CGGAGC, TACCGT, GCTTGC, or CTTGCT of exon 7; or GGTTCT or TCCTAC of exon 8 of the RHD gene.

In some embodiments, the deleterious variation of the FUT1 gene is generated using CRISPR-Cas9 gene editing comprising a guide RNA target sequence comprising one selected from the group consisting of those in Tables G-I.

In some embodiments, the deleterious variation comprises a deletion in exon 4 of the FUT1 gene and a guide RNA target sequence comprising SEQ ID NO:13 or SEQ ID NO:14.

In some embodiments, the deleterious variation of the FUT1 gene is a homozygous variation such that the cell has a Bombay phenotype.

In some embodiments, the deleterious variation of the RHD gene is generated by using CRISPR-Cas9 gene editing comprising one or more guide RNA target sequences comprising one or more selected from the group consisting of those in Tables D-F.

In some embodiments, the deleterious variation comprises a deletion of a genomic region comprising exons 1-8 of the RHD gene and a guide RNA target sequence comprising SEQ ID NO:11 and another guide RNA comprising SEQ ID NO:12.

In some embodiments, the deleterious variation of the RHD gene comprises a frame-shift insertion/deletion and a guide RNA target sequence comprises SEQ ID NO:9 or SEQ ID NO:10.

In some embodiments, the deleterious variation of the RHD gene is a homozygous variation such that the cell is Rhesus factor (Rh) negative.

In some embodiments, the deleterious variation of the RHD gene is a homozygous variation such that the cell has a Bombay phenotype and is Rh negative.

In some aspects, provided herein is an isolated cell in which expression of a FUT1 gene is partially or fully inactivated by a deleterious variation of the FUT1 gene. In some embodiments, the cell is further an Rh negative cell.

In some embodiments, expression of the FUT1 gene is partially or fully inactivated by an insertion or deletion within ATCGAC, AGTACG, CGCCCT, GTTTGC, CGACAA, GGTGCG, CGCCGT, ACGCCG, CCGGTT, CGCGGG, TTTTCG, ATACCG, GTGCGC, CCATTG, TGTCGG, ATCTGC, CTTTGT, GGGGCC, GGCCAT, TGCGAT, CGTGCA, or ATGGAC of exon 4 of the FUT1 gene.

In some embodiments, the deleterious variation of the FUT1 gene is generated using CRISPR-Cas9 gene editing comprising a guide RNA target sequence comprising one selected from the group consisting of those in Tables G-I.

In some embodiments, the deleterious variation comprises a deletion in exon 4 of the FUT1 gene and is generated using CRISPR-Cas9 gene editing comprising a guide RNA target sequence comprising SEQ ID NO:13 or SEQ ID NO:14.

In some embodiments, the deleterious variation of the FUT1 gene is a homozygous variation such that the cell is a universal Bombay negative cell.

In some aspects, provided herein is an isolated cell in which expression of an RHD gene is partially or fully inactivated by a deleterious variation of the RHD gene.

In some embodiments, the cell has a type O or Bombay phenotype.

In some embodiments, expression of the RHD gene is partially or fully inactivated by an insertion or deletion within TCATGG, GAGGTG, AACTCG, AGTTTC, TTGGCT, or CACAGC of exon 2; CCGTGA of exon 3; GGGTAG or AGGGAA of exon 4; TTCGAT, TCAGCG, CATAGT, or ATCGAA of exon 5; CGTCGG or TCCGTC of exon 6; CGGCAA, CGGAGC, TACCGT, GCTTGC, or CTTGCT of exon 7; or GGTTCT or TCCTAC of exon 8 of the RHD gene.

In some embodiments, the deleterious variation of the RHD gene is generated by using CRISPR-Cas9 gene editing comprising one or more guide RNA target sequences comprising one selected from the group consisting of those in Tables D-F.

In some embodiments, the deleterious variation comprises a deletion of a genomic region comprising exons 1-8 of the RHD gene and a guide RNA target sequence comprising SEQ ID NO:11 and another guide RNA comprising SEQ ID NO:12.

In some embodiments, the deleterious variation of the RHD gene comprises a frame-shift insertion/deletion and the guide RNA target sequence comprises SEQ ID NO:9 or SEQ ID NO:10.

In some embodiments, the deleterious variation of the RHD gene is a homozygous variation such that the cell is a type O negative cell or a Bombay negative cell.

In some aspects, provided herein is an isolated cell in which expression of an ABO gene is partially or fully inactivated by a deleterious variation of the ABO gene or by insertion of an exon 6 258delG variation of the ABO gene.

In some embodiments, expression of the ABO gene is partially or fully inactivated by an insertion or deletion within ACACCT of exon 1; AGTGCG of exon 2; or CAAGCGC, GGCGCA, TGGGCG, ACCACG, GCCGCA, CCACGT, TGCCGT, TACTCG, ACTCGG, GAGCGC, GCCTGG, GTCCTT, TCACGC, TGGACG, TGGTCG, GCTGGC, CACGCG, AGGTGA, or GCATCG of exon 8 of the ABO gene.

In some embodiments, the deleterious variation of the ABO gene is generated using CRISPR-Cas9 gene editing comprising a guide RNA target sequence comprising one selected from the group consisting of those in Tables A-C.

In some embodiments, the deleterious variation of the ABO gene comprises an insertion or deletion in a coding exon and the guide RNA target sequence comprises SEQ ID NO:1 or SEQ ID NO:2.

In some embodiments, the deleterious variation of the ABO gene comprises a frame-shift insertion/deletion and the guide RNA target sequence comprises SEQ ID NO:3.

In some embodiments, the deleterious variation of the ABO gene is a homozygous variation.

In some embodiments, the exon 6 258delG variation of the ABO gene is generated using CRISPR-Cas9 gene editing comprising a guide RNA target sequence comprising one selected from the group consisting of those in SEQ ID NOS:3-5 and a HDR template comprising a sequence encoding the 258delG variation, a PAM sequence, a spacer region, and two homology arms, each comprising about 10 nucleotides to about 1 Kb of homologous sequence to the ABO gene.

In some embodiments, the HDR template further comprises one or more silent mutations in the PAM sequence and/or the spacer region to ensure that the HDR template is not cleaved by Cas9.

In some embodiments, the exon 6 258delG variation of the ABO gene is generated using CRISPR-Cas9 gene editing comprising a guide RNA target sequence comprises one selected from the group consisting of SEQ ID NOS:3-5 and the HDR template comprises one selected from the group consisting of SEQ ID NOS:6-8.

In some embodiments, the exon 6 258delG variation of the ABO gene is a homozygous variation.

In some aspects, provided herein is an isolated cell in which expression of an RHD gene is partially or fully inactivated by a deleterious variation of the RHD gene.

In some embodiments, expression of the RHD gene is partially or fully inactivated by an insertion or deletion within TCATGG, GAGGTG, AACTCG, AGTTTC, TTGGCT, or CACAGC of exon 2; CCGTGA of exon 3; GGGTAG or AGGGAA of exon 4; TTCGAT, TCAGCG, CATAGT, or ATCGAA of exon 5; CGTCGG or TCCGTC of exon 6; CGGCAA, CGGAGC, TACCGT, GCTTGC, or CTTGCT of exon 7; or GGTTCT or TCCTAC of exon 8 of the RHD gene.

In some embodiments, the deleterious variation of the RHD gene is generated by using CRISPR-Cas9 gene editing comprising one or more guide RNA target sequences comprising one or more selected from the group consisting of those in Tables D-F.

In some embodiments, the deleterious variation comprises a deletion of a genomic region comprising exons 1-8 of the RHD gene and a guide RNA target sequence comprising SEQ ID NO:11 and another guide RNA comprising SEQ ID NO:12.

In some embodiments, the deleterious variation of the RHD gene comprises a frame-shift insertion/deletion and a guide RNA target sequence comprises SEQ ID NO:9 or SEQ ID NO:10.

In some embodiments, the deleterious variation of the RHD gene is a homozygous variation.

In some aspects, provided herein is an isolated cell in which expression of a FUT1 gene is partially or fully inactivated by a deleterious variation of the FUT1 gene.

In some embodiments, expression of the FUT1 gene is partially or fully inactivated by an insertion or deletion within ATCGAC, AGTACG, CGCCCT, GTTTGC, CGACAA, GGTGCG, CGCCGT, ACGCCG, CCGGTT, CGCGGG, TTTTCG, ATACCG, GTGCGC, CCATTG, TGTCGG, ATCTGC, CTTTGT, GGGGCC, GGCCAT, TGCGAT, CGTGCA, or ATGGAC of exon 4 of the FUT1 gene.

In some embodiments, the deleterious variation of the FUT1 gene is generated using CRISPR-Cas9 gene editing comprising a guide RNA target sequence comprising one selected from the group consisting of those in Tables G-I.

In some embodiments, the deleterious variation comprises a deletion in exon 4 of the FUT1 gene and a guide RNA target sequence comprising SEQ ID NO:13 or SEQ ID NO:14.

In some embodiments, the deleterious variation of the RHD gene is a homozygous variation.

In some embodiments, the cell is a human cell. In further embodiments, the cell is selected from the group consisting of an induced pluripotent stem cell, an embryonic stem cell (such as RUES2 or H9 cells), an adult stem cell, and a differentiated cell.

In some embodiments, the cell comprises one or more partially or fully inactivated genes encoding one or more transcriptional regulators of MHC-I and/or one or more partially or fully inactivated genes encoding one or more transcriptional regulators of MHC-II.

In some embodiments, expression of a B2M gene is partially or fully inactivated.

In some embodiments, expression of a CIITA gene is partially or fully inactivated.

In some embodiments, the cell further comprises a CD47 transgene.

In some embodiments, expression of the B2M and CIITA genes are partially or fully inactivated and the cell further comprises a CD47 transgene.

In some embodiments, provided herein is a pharmaceutical composition comprising a cell in accordance with any of the above.

In some embodiments, provided herein is a method for treating a patient in need to a cell replacement therapy comprising administering a cell in accordance with any of the above.

In some aspects, provided herein is a method of generating an engineered histocompatible cell, the method comprising: (a) obtaining an isolated cell; (b) introducing a Cas9 nuclease and a guide RNA target sequence for an ABO gene comprising one selected from the group consisting of those in Tables A-C into the cell; and (c) selecting an engineered cell in which the ABO gene is partially or fully inactivated.

In some embodiments, the guide RNA target sequence comprises one selected from the group consisting of SEQ ID NOS:1-3.

In some embodiments, the method further comprises the steps of (i) introducing a guide RNA target sequence for an RHD gene comprising one selected from the group consisting of those in Tables D-F into the cell; and (ii) selecting an engineered cell in which the RHD gene is partially or fully inactivated.

In some embodiments, the guide RNA target sequence comprising SEQ ID NO:11 and the another guide RNA comprising SEQ ID NO:12. In some embodiments, the guide RNA target sequence comprises SEQ ID NO:9 or SEQ ID NO:10.

In some further aspects, provided herein is a method of generating an engineered histocompatible cell, the method comprising: (a) obtaining an isolated cell; (b) introducing a Cas9 nuclease and a guide RNA target sequence for producing an exon 6 258delG variation of an ABO gene comprising one selected from the group consisting of those in SEQ ID NOS:3-5 and a HDR template comprising a sequence encoding the 258delG variation, a PAM sequence, a spacer region, and two homology arms, each comprising about 10 nucleotides to about 1 Kb of homologous sequence to the ABO gene into the cell; and (c) selecting an engineered cell in which the ABO gene is partially or fully inactivated.

In some embodiments, the HDR template further comprises one or more silent mutations in the PAM sequence and/or the spacer region to ensure that the HDR template is not cleaved by Cas9.

In some embodiments, the guide RNA target sequence comprises one selected from the group consisting of SEQ ID NOS:3-5 and the HDR template comprises one selected from the group consisting of SEQ ID NOS:6-8.

In some embodiments, the method of generating the engineered histocompatible cell further includes the steps of (i) introducing a guide RNA target sequence for an RHD gene comprising one selected from the group consisting of those in Tables D-F into the cell; and (ii) selecting an engineered cell in which the RHD gene is partially or fully inactivated.

In some embodiments, the guide RNA target sequence comprising SEQ ID NO:11 and the another guide RNA comprising SEQ ID NO:12.

In some embodiments, the guide RNA target sequence comprises SEQ ID NO:9 or SEQ ID NO:10.

In some aspects, provided herein is a method for generating an engineered histocompatible cell, the method comprising: (a) obtaining an isolated cell; (b) introducing a Cas9 nuclease and a guide RNA target sequence for an RHD gene comprising one selected from the group consisting of those in Tables D-F into the cell; and (c) selecting an engineered cell in which the RHD gene is partially or fully inactivated.

In some embodiments, the guide RNA target sequence comprising SEQ ID NO:11 and the another guide RNA comprising SEQ ID NO:12.

In some embodiments, the guide RNA target sequence comprises SEQ ID NO:9 or SEQ ID NO:10.

In some embodiments, the method for generating an engineered histocompatible cell further comprises the steps of: (i) introducing a guide RNA target sequence for an ABO gene comprising one selected from the group consisting of those in Tables A-C into the cell; and (ii) selecting an engineered cell in which the ABO gene is partially or fully inactivated. In some embodiments, the guide RNA target sequence comprises SEQ ID NOS:1-3.

In some embodiments, the method for generating an engineered histocompatible cell further comprises the steps of; (i) introducing a guide RNA target sequence for producing an exon 6 258delG variation of an ABO gene comprising one selected from the group consisting of those in SEQ ID NOS:3-5 and a HDR template comprising a sequence encoding the 258delG variation, a PAM sequence, a spacer region, and two homology arms, each comprising about 10 nucleotides to about 1 Kb of homologous sequence to the ABO gene into the cell; and (ii) selecting an engineered cell in which the ABO gene is partially or fully inactivated.

In some embodiments, the HDR template further comprises one or more silent mutations in the PAM sequence and/or the spacer region to ensure that the HDR template is not cleaved by Cas9.

In some embodiments, the guide RNA target sequence comprises one selected from the group consisting of SEQ ID NOS:3-5 and the HDR template comprises one selected from the group consisting of SEQ ID NOS:6-8. In some aspects, provided herein is a method of generating an engineered histocompatible cell, the method comprising: (a) obtaining an isolated cell; (b) introducing a Cas9 nuclease and a guide RNA target sequence for a FUT1 gene comprising one selected from the group consisting of those in Tables G-I into the cell; and (c) selecting an engineered cell in which the FUT1 gene is partially or fully inactivated.

In some embodiments, the guide RNA target sequence comprising SEQ ID NO:13 or SEQ ID NO:14.

In some embodiments, the method of generating an engineered histocompatible cell further comprises the steps of: (i) introducing a guide RNA target sequence for an RHD gene comprising one selected from the group consisting of those in Tables D-F into the cell; and (ii) selecting an engineered cell in which the RHD gene is partially or fully inactivated.

In some embodiments, the guide RNA target sequence comprises SEQ ID NO:9 or SEQ ID NO:10.

In some embodiments, the guide RNA target sequence comprises SEQ ID NO:11 and another guide RNA target sequence comprises SEQ ID NO:12.

In some embodiments, the isolated cell is an isolated human cell.

In some embodiments, the isolated cell is selected from the group consisting of a pluripotent stem cell, an induced pluripotent stem cell, an embryonic stem cell (such as RUES2 or H9 cells), a multipotent stem cell, an adult stem cell, and a differentiated cell.

In some embodiments, the isolated cell comprises one or more partially or fully inactivated genes encoding one or more transcriptional regulators of MHC-I and/or one or more partially or fully inactivated genes encoding one or more transcriptional regulators of MHC-II.

In some embodiments, the isolated cell comprises a partially or fully inactivated B2M gene.

In some further embodiments, the isolated cell used in accordance with the above method further comprises a partially or fully inactivated CIITA gene. In some further embodiments, the isolated cell comprises a CD47 transgene. In some further embodiments, the isolated cell comprises partially or fully inactivated B2M and CIITA genes and a CD47 transgene.

In some further aspects, provided herein is a method of preparing a differentiated cell, the method comprising culturing under differentiation conditions the stem cell prepared according to the above method, thereby preparing a differentiated cell. In some embodiments, the differentiation conditions are appropriate for differentiation of a stem cell into a cell type selected from the group consisting of cardiac cells, liver cell, kidney cells, pancreatic cells, neural cells, immune cells, mesenchymal cells, and endothelial cells.

In some further aspects, provided herein is a method of treating a patient in need of cell replacement therapy, the method comprising administering a population of differentiated cells prepared according to the above method.

In some aspects, provided herein is an isolated cell derived from a RUES2 cell line, wherein the isolated cell comprises one or both of: (a) a modification that renders the isolated cell histo-blood group O; and (b) a modification that renders the isolated cell Rh negative.

In some further embodiments, the modification that renders the isolated cell histo-blood group O reduces or eliminates antigenicity of the ABO histo-blood group B antigen.

In some further embodiments, wherein the modification comprises partially or fully inactivated expression of an ABO gene by a deleterious variation of the ABO gene or by insertion of an exon 6 258delG variation of the ABO gene.

In some further embodiments, expression of the ABO gene is partially or fully inactivated by an insertion or deletion within ACACCT of exon 1; AGTGCG of exon 2; or CAAGCGC, GGCGCA, TGGGCG, ACCACG, GCCGCA, CCACGT, TGCCGT, TACTCG, ACTCGG, GAGCGC, GCCTGG, GTCCTT, TCACGC, TGGACG, TGGTCG, GCTGGC, CACGCG, AGGTGA, or GCATCG of exon 8 of the ABO gene.

In some further embodiments, the deleterious variation of the ABO gene is generated using CRISPR-Cas9 gene editing comprising a guide RNA target sequence comprising one selected from the group consisting of those in Tables A-C.

In some further embodiments, the deleterious variation of the ABO gene comprises an insertion or deletion in a coding exon and the guide RNA target sequence comprises SEQ ID NO:1 or SEQ ID NO:2.

In some further embodiments, the deleterious variation of the ABO gene comprises a frame-shift insertion/deletion and the guide RNA target sequence comprises SEQ ID NO:3.

In some further embodiments, the deleterious variation of the ABO gene is a homozygous variation.

In some further embodiments, the exon 6 258delG variation of the ABO gene is generated using CRISPR-Cas9 gene editing comprising a guide RNA target sequence comprising one selected from the group consisting of those in SEQ ID NOS:3-5 and a HDR template comprising a sequence encoding the 258delG variation, a PAM sequence, a spacer region, and two homology arms, each comprising about 10 nucleotides to about 1 Kb of homologous sequence to the ABO gene.

In some further embodiments, the HDR template further comprises one or more silent mutations in the PAM sequence and/or the spacer region to ensure that the HDR template is not cleaved by Cas9.

In some further embodiments, wherein the exon 6 258delG variation of the ABO gene is generated using CRISPR-Cas9 gene editing comprising a guide RNA target sequence comprises one selected from the group consisting of SEQ ID NOS:3-5 and the HDR template comprises one selected from the group consisting of SEQ ID NOS:6-8.

In some further embodiments, the exon 6 258delG variation of the ABO gene is a homozygous variation.

In some further embodiments, the modification that renders the isolated cell Rh negative comprises partially or fully inactivated expression of a FUT1 gene by a deleterious variation of the FUT1 gene.

In some further embodiments, the expression of the FUT1 gene is partially or fully inactivated by an insertion or deletion within ATCGAC, AGTACG, CGCCCT, GTTTGC, CGACAA, GGTGCG, CGCCGT, ACGCCG, CCGGTT, CGCGGG, TTTTCG, ATACCG, GTGCGC, CCATTG, TGTCGG, ATCTGC, CTTTGT, GGGGCC, GGCCAT, TGCGAT, CGTGCA, or ATGGAC of exon 4 of the FUT1 gene.

In some further embodiments, the deleterious variation of the FUT1 gene is generated using CRISPR-Cas9 gene editing comprising a guide RNA target sequence comprising one selected from the group consisting of those in Tables G-I.

In some further embodiments, the deleterious variation comprises a deletion in exon 4 of the FUT1 gene and a guide RNA target sequence comprising SEQ ID NO:13 or SEQ ID NO:14.

In some further embodiments, the modification that renders the isolated cell Rh negative reduces or eliminates antigenicity of one or more Rhesus factor antigens.

In some further embodiments, the modification comprises partially or fully inactivated expression of an RHD gene by a deleterious variation of the RHD gene.

In some further embodiments, expression of the RHD gene is partially or fully inactivated by an insertion or deletion within TCATGG, GAGGTG, AACTCG, AGTTTC, TTGGCT, or CACAGC of exon 2; CCGTGA of exon 3; GGGTAG or AGGGAA of exon 4; TTCGAT, TCAGCG, CATAGT, or ATCGAA of exon 5; CGTCGG or TCCGTC of exon 6; CGGCAA, CGGAGC, TACCGT, GCTTGC, or CTTGCT of exon 7; or GGTTCT or TCCTAC of exon 8 of the RHD gene.

In some further embodiments, the deleterious variation of the RHD gene is generated by using CRISPR-Cas9 gene editing comprising one or more guide RNA target sequences comprising one selected from the group consisting of those in Tables D-F.

In some further embodiments, the deleterious variation comprises a deletion of a genomic region comprising exons 1-8 of the RHD gene and a guide RNA target sequence comprising SEQ ID NO:11 and another guide RNA comprising SEQ ID NO:12.

In some further embodiments, the deleterious variation of the RHD gene comprises a frame-shift insertion/deletion and the guide RNA target sequence comprises SEQ ID NO:9 or SEQ ID NO:10.

In some embodiments, the isolated cell described above further comprises one or more partially or fully inactivated genes encoding one or more transcriptional regulators of MHC-I and/or one or more partially or fully inactivated genes encoding one or more transcriptional regulators of MHC-II.

In some embodiments, the isolated cell comprises a partially or fully inactivated B2M gene. In some further embodiments, the isolated cell used in accordance with the above method further comprises a partially or fully inactivated CIITA gene. In some further embodiments, the isolated cell comprises a CD47 transgene. In some further embodiments, the isolated cell comprises partially or fully inactivated B2M and CIITA genes and a CD47 transgene.

In some aspects, provided herein is a method of generating an engineered histocompatible cell comprising: (a) obtaining an isolated RUES2 cell or a derivative thereof; (b) introducing a Cas9 nuclease and a guide RNA target sequence for an ABO gene comprising one selected from the group consisting of those in Tables A-C into the cell; and (c) selecting an engineered cell in which the ABO gene is partially or fully inactivated.

In some further embodiments, the guide RNA target sequence comprises one selected from the group consisting of SEQ ID NOS:1-3.

In some further embodiments, the method for generating the engineered histocompatible cell further comprises the steps of: (i) introducing a guide RNA target sequence for an RHD gene comprising one selected from the group consisting of those in Tables D-F into the cell; and (ii) selecting an engineered cell in which the RHD gene is partially or fully inactivated.

In some further embodiments, the guide RNA target sequence comprising SEQ ID NO:11 and the another guide RNA comprising SEQ ID NO:12.

In some further embodiments, the guide RNA target sequence comprises SEQ ID NO:9 or SEQ ID NO:10.

In some aspects, provided herein is a method of generating an engineered histocompatible cell, the method comprising: (a) obtaining an isolated RUES2 cell or a derivative thereof; (b) introducing a Cas9 nuclease and a guide RNA target sequence for producing an exon 6 258delG variation of an ABO gene comprising one selected from the group consisting of those in SEQ ID NOS:3-5 and a HDR template comprising a sequence encoding the 258delG variation, a PAM sequence, a spacer region, and two homology arms, each comprising about 10 nucleotides to about 1 Kb of homologous sequence to the ABO gene into the cell; and (c) selecting an engineered cell in which the ABO gene is partially or fully inactivated.

In some further embodiments, the HDR template further comprises one or more silent mutations in the PAM sequence and/or the spacer region to ensure that the HDR template is not cleaved by Cas9.

In some further embodiments, the guide RNA target sequence comprises one selected from the group consisting of SEQ ID NOS:3-5 and the HDR template comprises one selected from the group consisting of SEQ ID NOS:6-8.

In some further embodiments, the method further comprises the steps: (i) introducing a guide RNA target sequence for an RHD gene comprising one selected from the group consisting of those in Tables D-F into the cell; and (ii) selecting an engineered cell in which the RHD gene is partially or fully inactivated.

In some further embodiments, the guide RNA target sequence comprising SEQ ID NO:11 and the another guide RNA comprising SEQ ID NO:12.

In some further embodiments, the guide RNA target sequence comprises SEQ ID NO:9 or SEQ ID NO:10.

In some aspects, provided herein is a method of generating an engineered histocompatible cell comprising: (a) obtaining an isolated RUES2 cell or a derivative thereof; (b) introducing a Cas9 nuclease and a guide RNA target sequence for an RHD gene comprising one selected from the group consisting of those in Tables D-F into the cell; and (c) selecting an engineered cell in which the RHD gene is partially or fully inactivated.

In some further embodiments, the guide RNA target sequence comprising SEQ ID NO: 11 and the another guide RNA comprising SEQ ID NO:12.

In some further embodiments, the guide RNA target sequence comprises SEQ ID NO:9 or SEQ ID NO:10.

In some further embodiments, the method further comprises the steps of: (i) introducing a guide RNA target sequence for an ABO gene comprising one selected from the group consisting of those in Tables A-C into the cell; and (ii) selecting an engineered cell in which the ABO gene is partially or fully inactivated.

In some further embodiments, the guide RNA target sequence comprises SEQ ID NOS:1-3.

In some further embodiments, the method further comprises the steps of: (i) introducing a guide RNA target sequence for producing an exon 6 258delG variation of an ABO gene comprising one selected from the group consisting of those in SEQ ID NOS:3-5 and a HDR template comprising a sequence encoding the 258delG variation, a PAM sequence, a spacer region, and two homology arms, each comprising about 10 nucleotides to about 1 Kb of homologous sequence to the ABO gene into the cell; and (ii) selecting an engineered cell in which the ABO gene is partially or fully inactivated.

In some further embodiments, the HDR template further comprises one or more silent mutations in the PAM sequence and/or the spacer region to ensure that the HDR template is not cleaved by Cas9.

In some further embodiments, the guide RNA target sequence comprises one selected from the group consisting of SEQ ID NOS:3-5 and the HDR template comprises one selected from the group consisting of SEQ ID NOS:6-8.

In some aspects, provided herein is a method of generating an engineered histocompatible cell, the method comprising: (a) obtaining an isolated RUES2 cell or a derivative thereof; (b) introducing a Cas9 nuclease and a guide RNA target sequence for a FUT1 gene comprising one selected from the group consisting of those in Tables G-I into the cell; and (c) selecting an engineered cell in which the FUT1 gene is partially or fully inactivated.

In some further embodiments, the guide RNA target sequence comprising SEQ ID NO:13 or SEQ ID NO:14.

In some further embodiments, the method further includes the steps of (i) introducing a guide RNA target sequence for an RHD gene comprising one selected from the group consisting of those in Tables D-F into the cell; and (ii) selecting an engineered cell in which the RHD gene is partially or fully inactivated.

In some further embodiments, the guide RNA target sequence comprises SEQ ID NO:9 or SEQ ID NO:10.

In some further embodiments, the guide RNA target sequence comprises SEQ ID NO:11 and another guide RNA target sequence comprises SEQ ID NO:12.

In some further embodiments, the isolated cell further comprises one or more partially or fully inactivated genes encoding one or more transcriptional regulators of MHC-I and/or one or more partially or fully inactivated genes encoding one or more transcriptional regulators of MHC-II.

In some further embodiments, the isolated cell comprises a partially or fully inactivated B2M gene.

In some further embodiments, the isolated cell comprises a partially or fully inactivated CIITA gene.

In some further embodiments, the isolated cell comprises a CD47 transgene. In some further embodiments, the isolated cell comprises partially or fully inactivated B2M and CIITA genes and a CD47 transgene.

In some aspects, provided herein is a method of preparing a differentiated cell comprising culturing under differentiation conditions the engineered histocompatible cell prepared according to any of the methods described above, thereby preparing a differentiated cell.

In some further embodiments, the differentiation conditions are appropriate for differentiation of a stem cell into a cell type selected from the group consisting of cardiac cells, liver cell, kidney cells, pancreatic cells, neural cells, immune cells, mesenchymal cells, and endothelial cells.

In some aspects, provided herein is a method of treating a patient in need of cell replacement therapy comprising administering a population of differentiated cells prepared according to any of the methods described above.

Detailed descriptions of hypoimmunogenic cells, methods of producing thereof, and methods of using thereof are found in WO2016183041 filed May 9, 2015 and WO2018132783 filed Jan. 14, 2018, the disclosures including the sequence listings and Figures are incorporated herein by reference in their entirety.

Other objects, advantages and embodiments of the invention will be apparent from the detailed description following.

### BRIEF DESCRIPTION OF THE DRAWINGS

FIG. 1 shows exemplary embodiments of a universal O negative iPSCs.

FIG. 2 shows exemplary genome modification strategies for the ABO gene that can be used to generate universal O negative cells.

FIG. 3 shows exemplary genome modification strategies for the RHD gene that can be used to generate universal O negative cells.

FIG. 4 shows 3 HDR strategies to produce c.258delG ABO genotypes.

FIGS. 5A-5AE show exemplary ABO target sequences and the genomic structure of the ABO gene.

FIGS. 6A-6C show exemplary ABO 238delG donor templates and guide RNA target sequences.

FIGS. 7A-7K show exemplary FUT1 target sequences and the genomic structure of the FUT1 gene.

FIGS. 8A-8BR show exemplary RHD target sequences and the genomic structure of the RHD gene.

FIG. 9 shows Table A of exemplary ABO gRNA target sequences.

FIGS. 10A-10E show Table B of exemplary ABO gRNA target sequences to target coding exons.

FIGS. 11A-BZ show Table C of ABO gRNA target sequences.

FIG. 12 shows Table D of exemplary RHD gRNA target sequences.

FIGS. 13A-13D show Table E of exemplary RHD gRNA target sequences to target coding exons.

FIGS. 14A-14FE show Table F of RHD gRNA target sequences.

FIG. 15 shows Table G of exemplary FUT1 gRNA target sequences.

FIG. 16A-16L show Table H of exemplary FUT1 gRNA target sequences to target coding exons.

FIGS. 17A-17AD show Table I of FUT1 gRNA target sequences.

FIG. 18 shows RUES2-cardiomyocytes survival when incubated with blood type matched monkey serum, and rejection when incubated with mismatched blood.

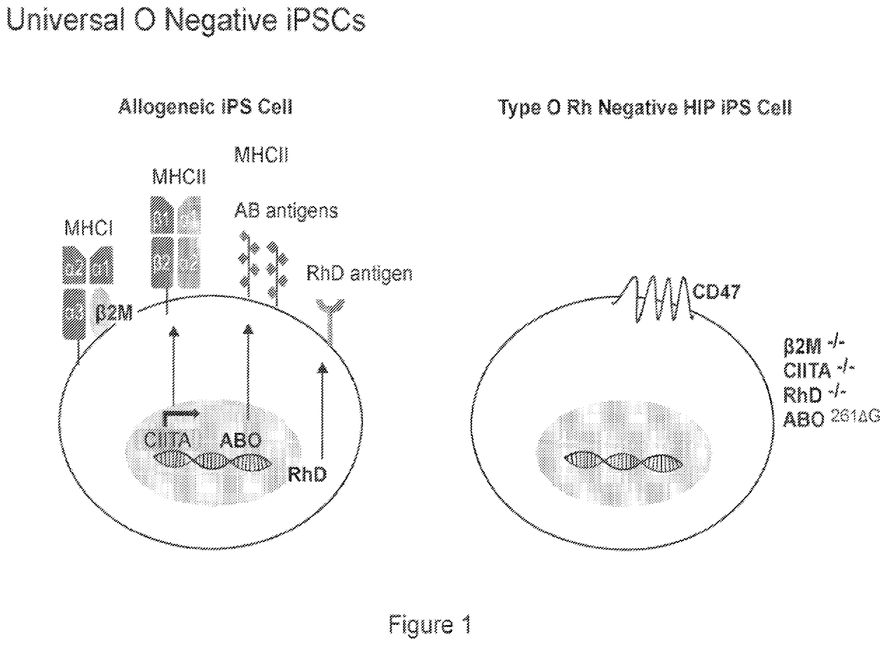

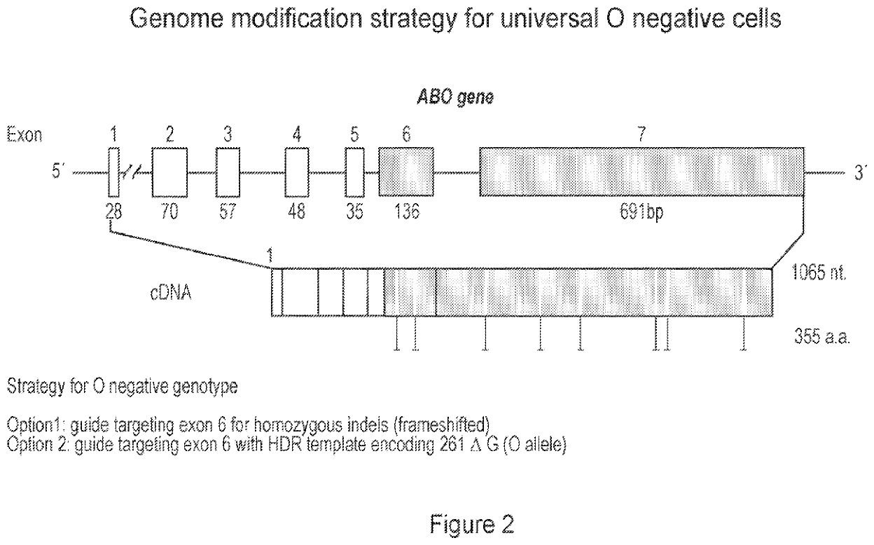

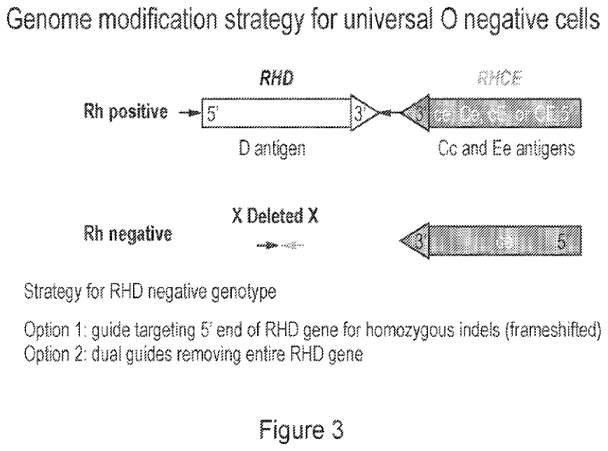

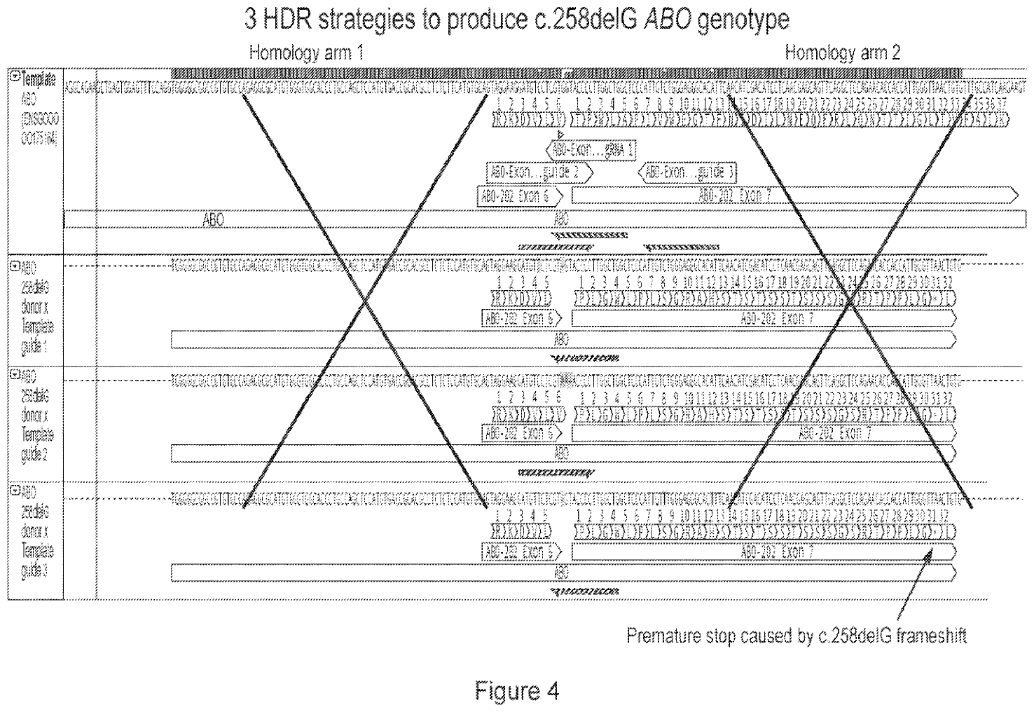

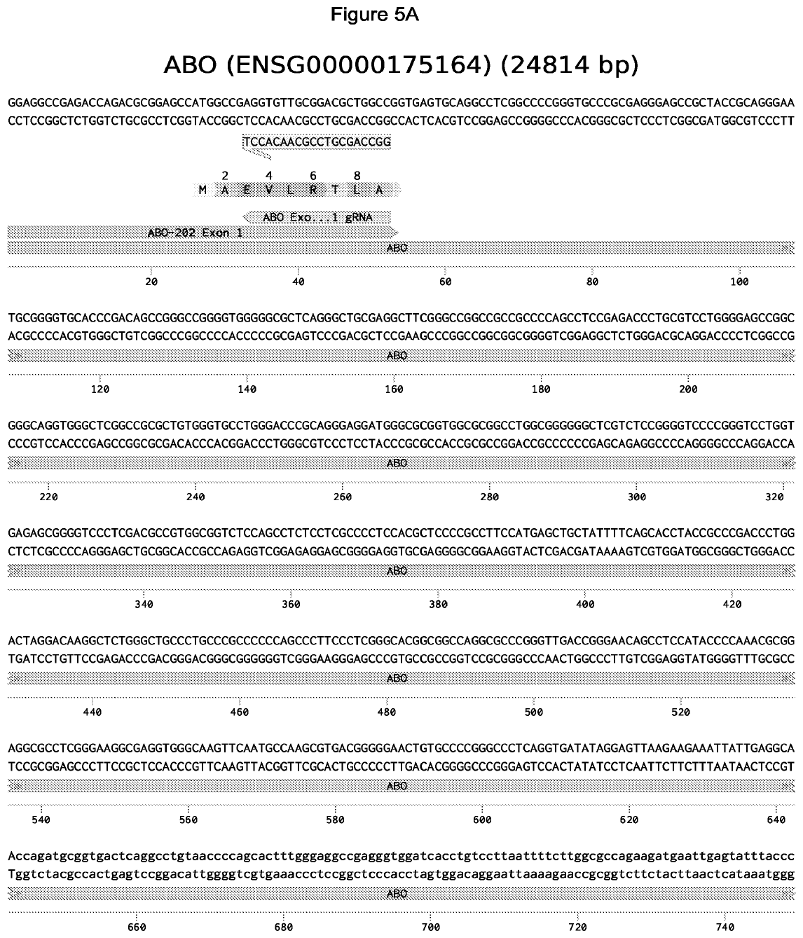

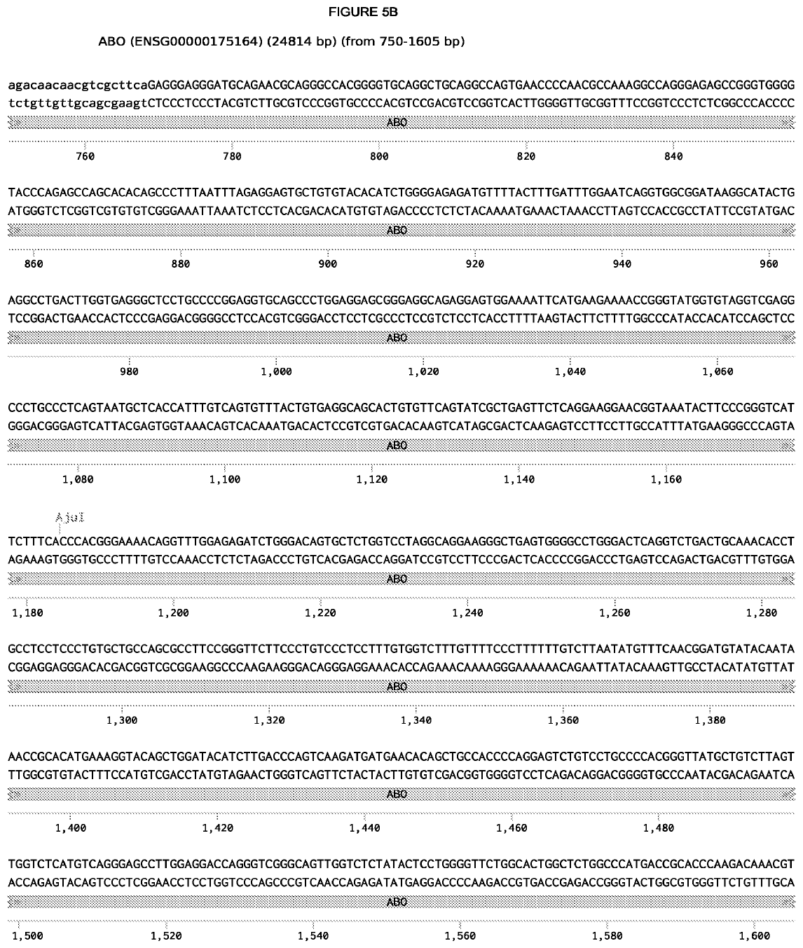

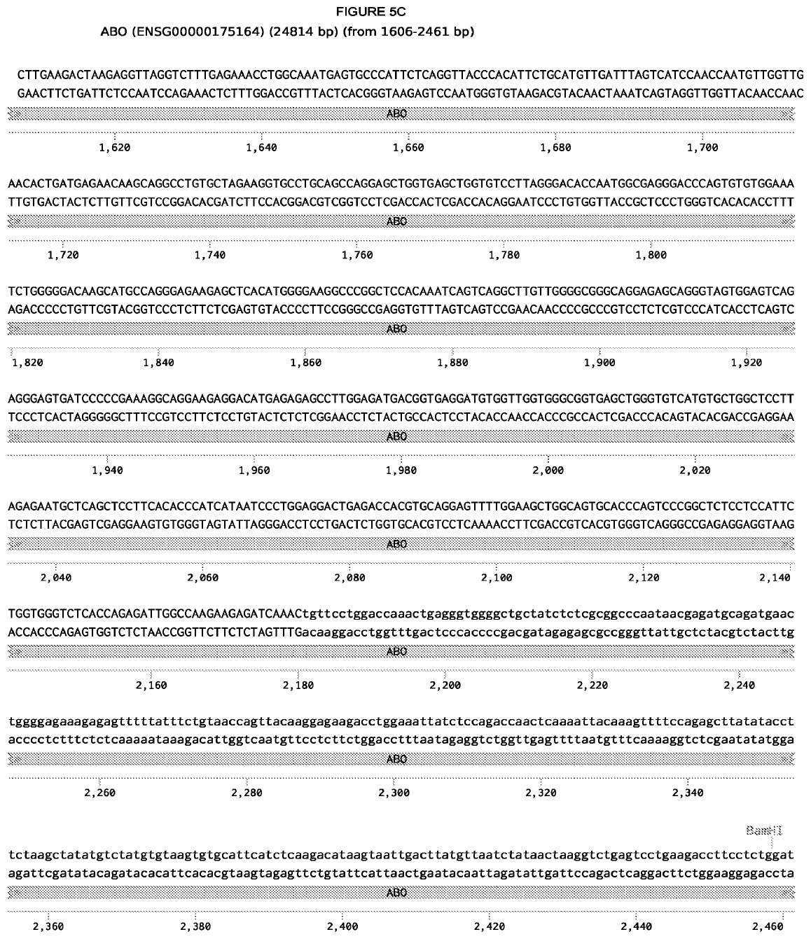

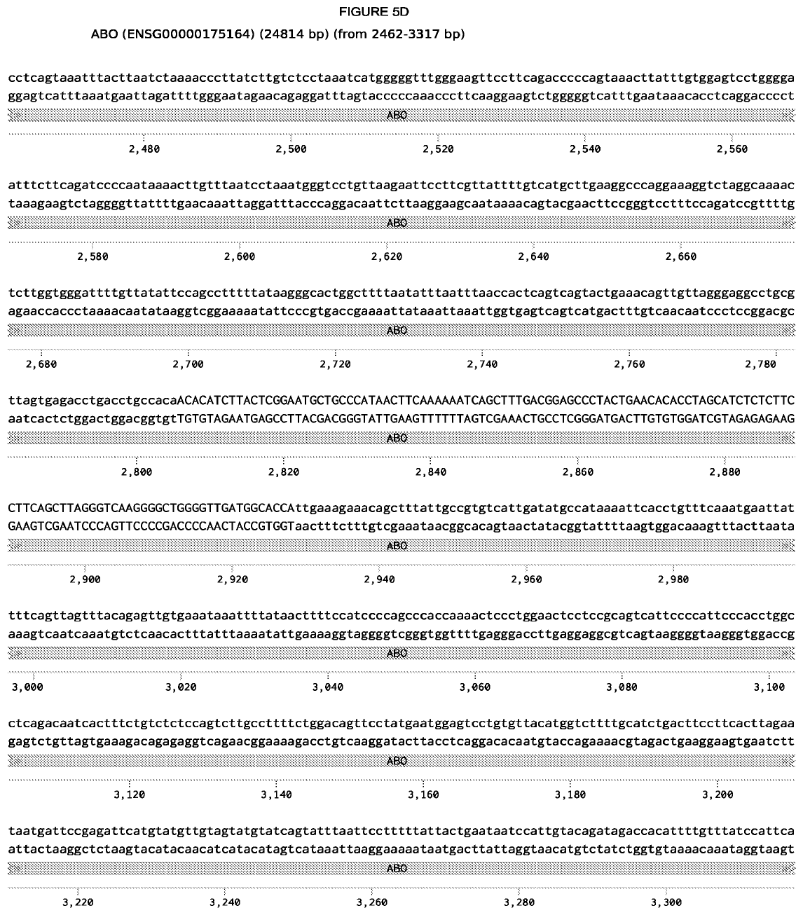

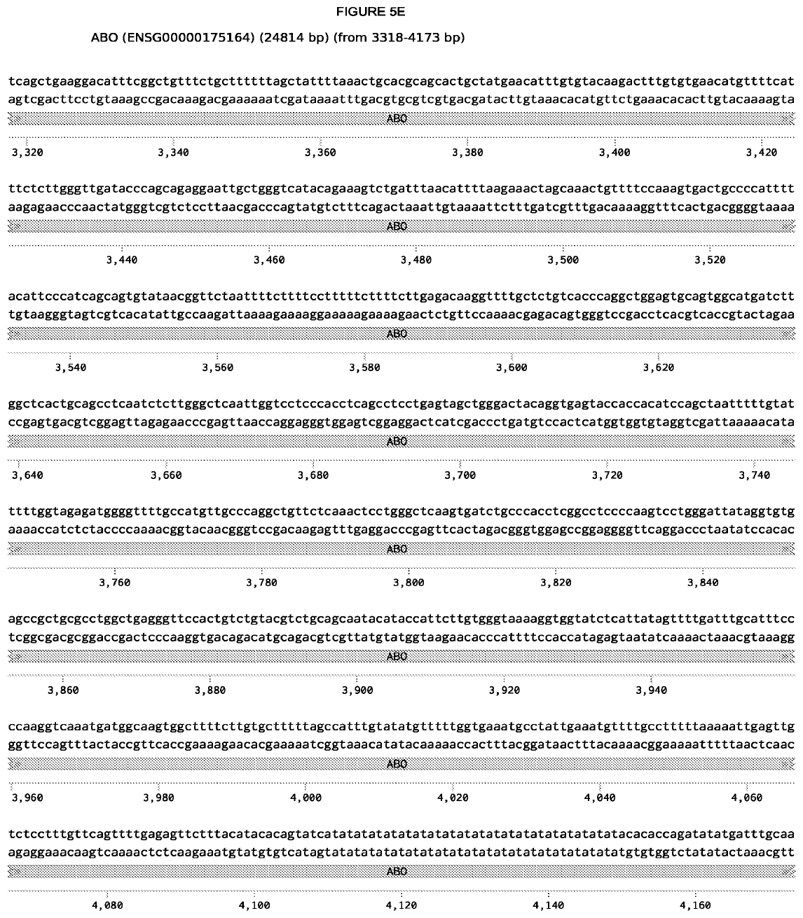

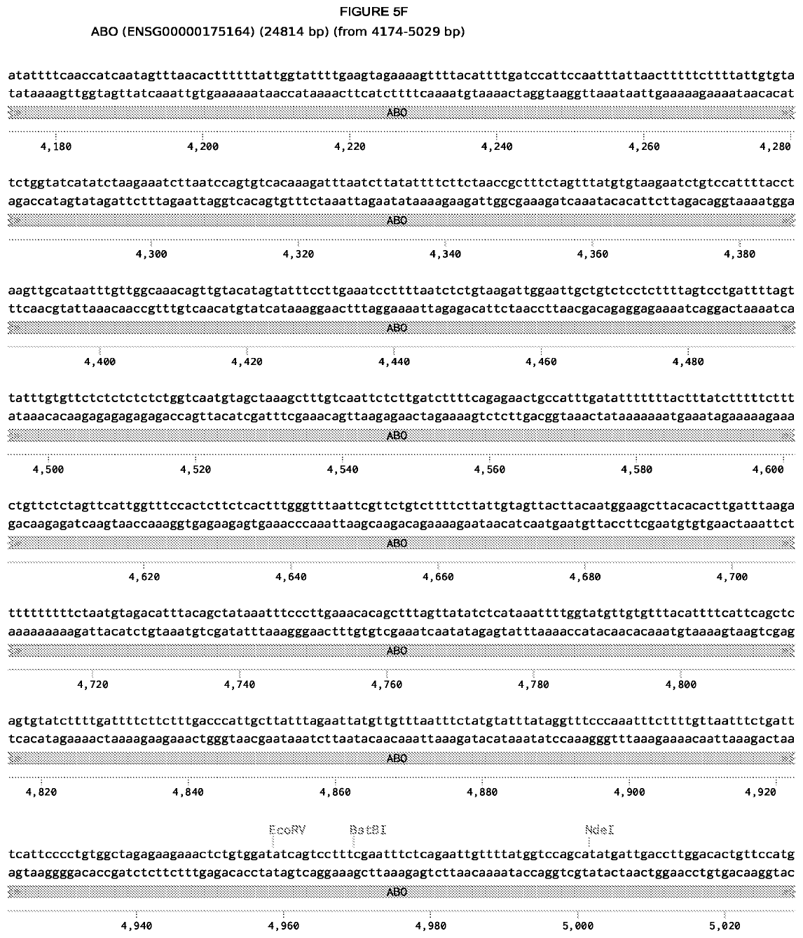

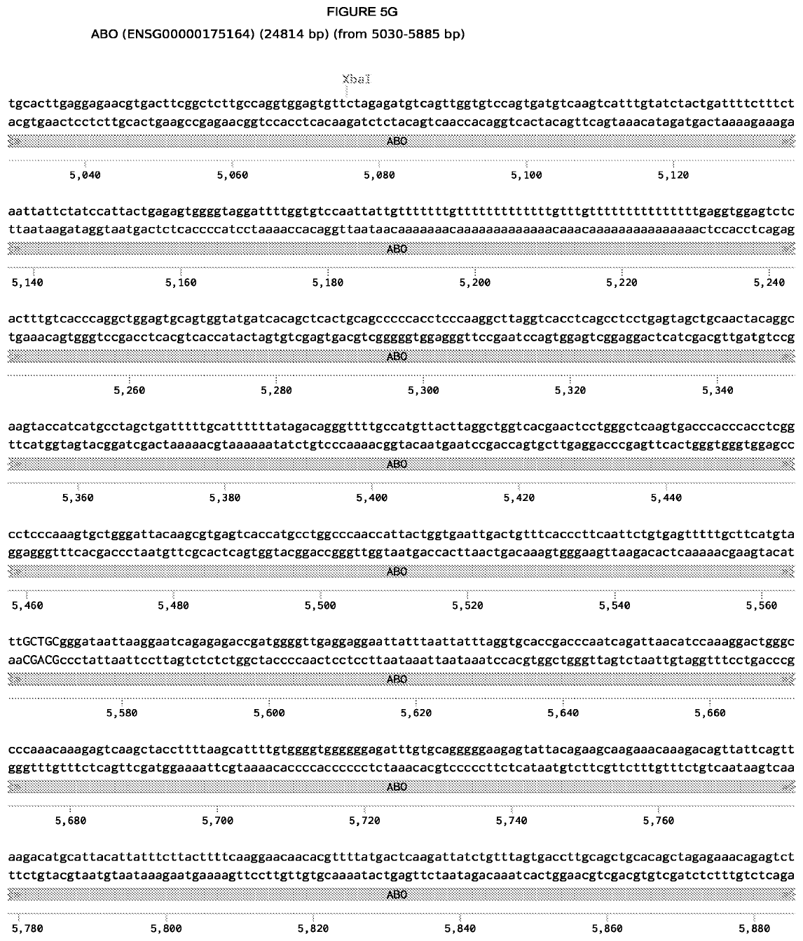

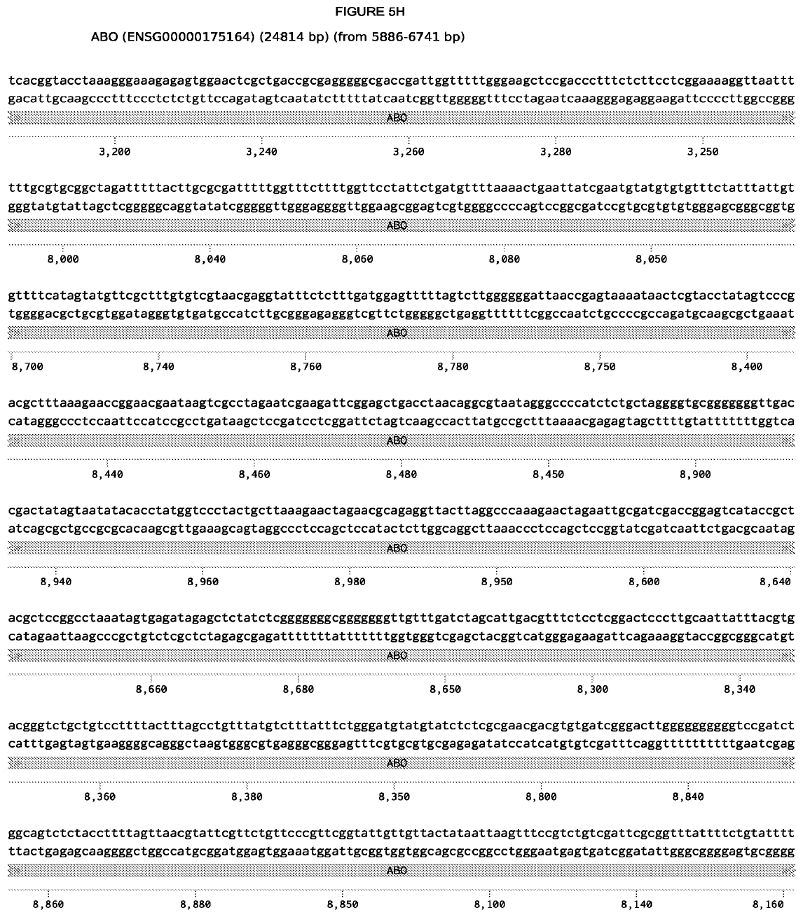

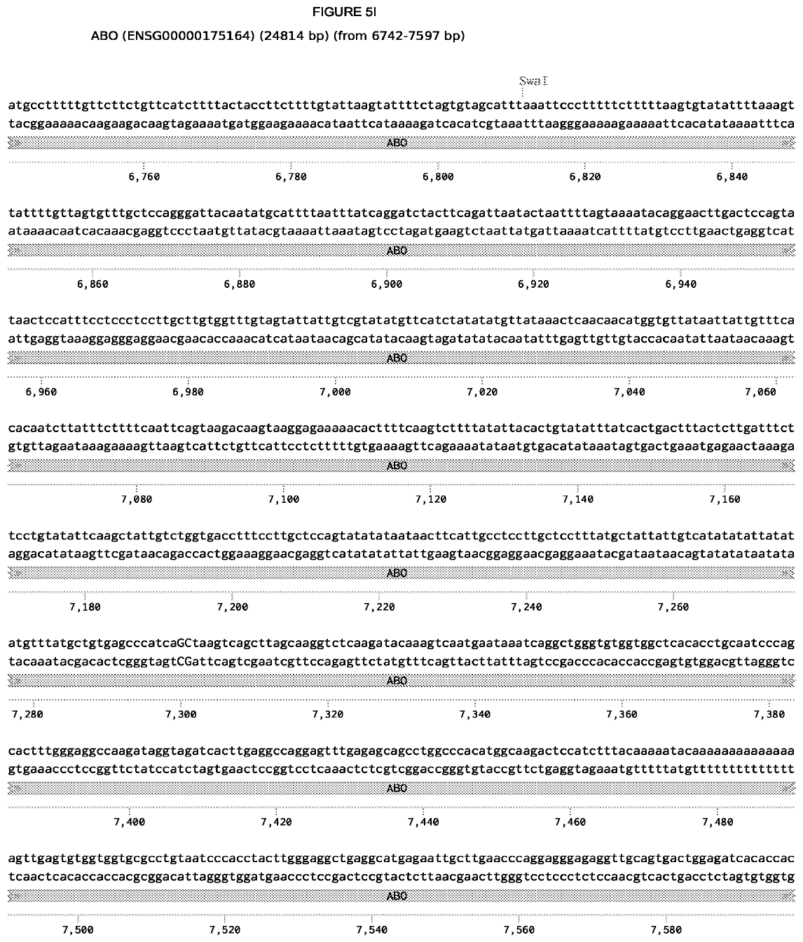

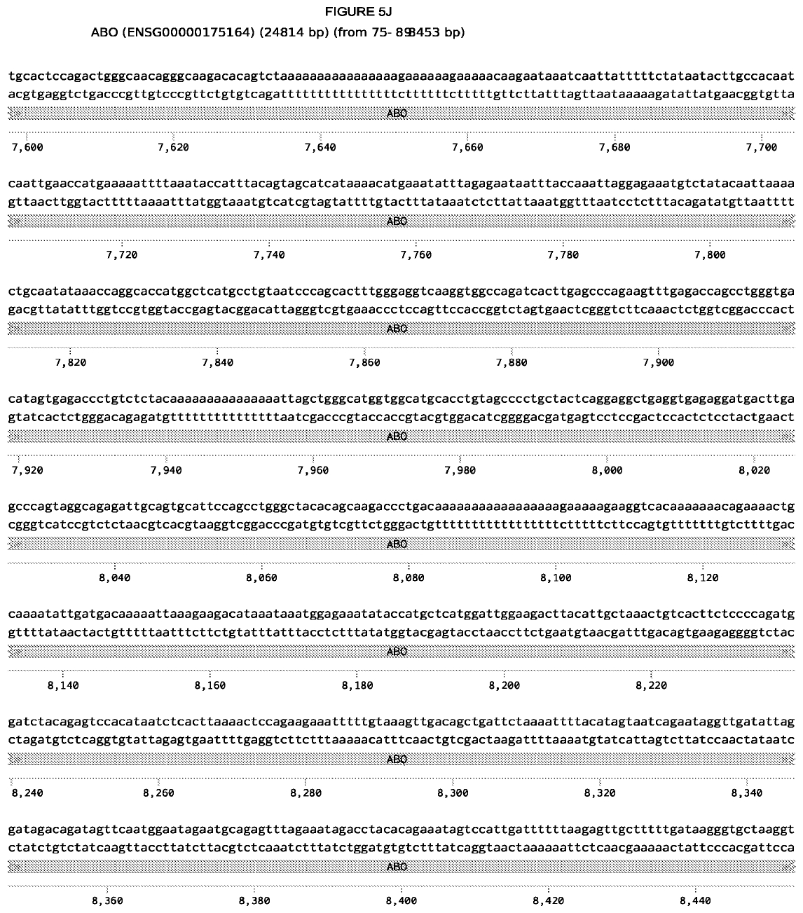

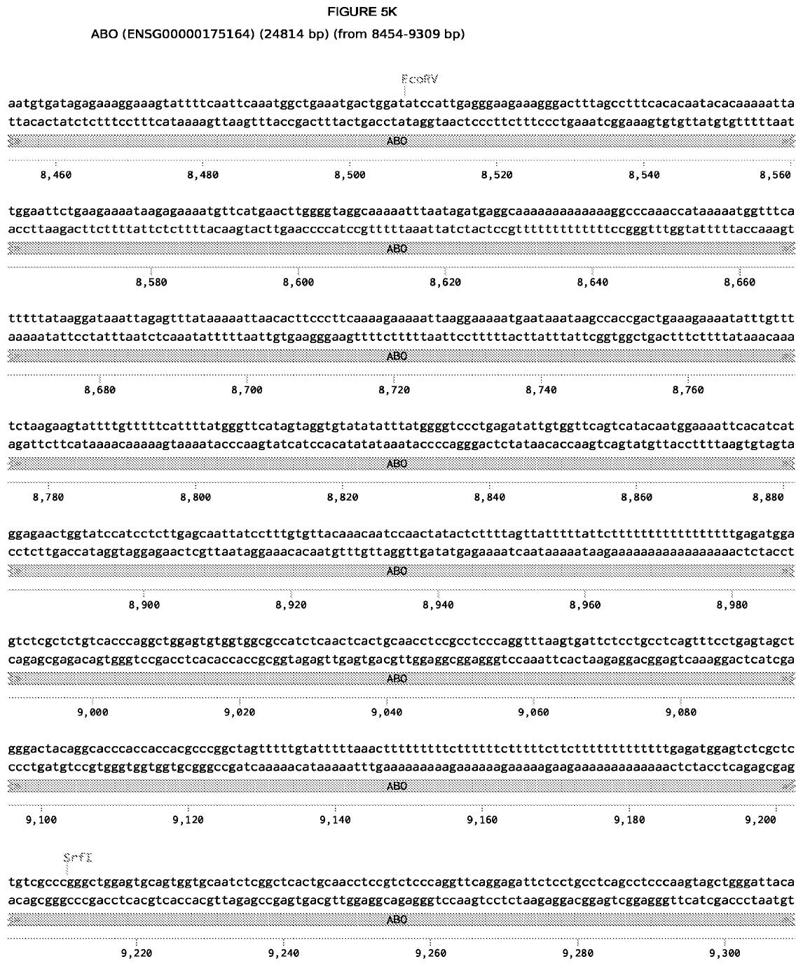

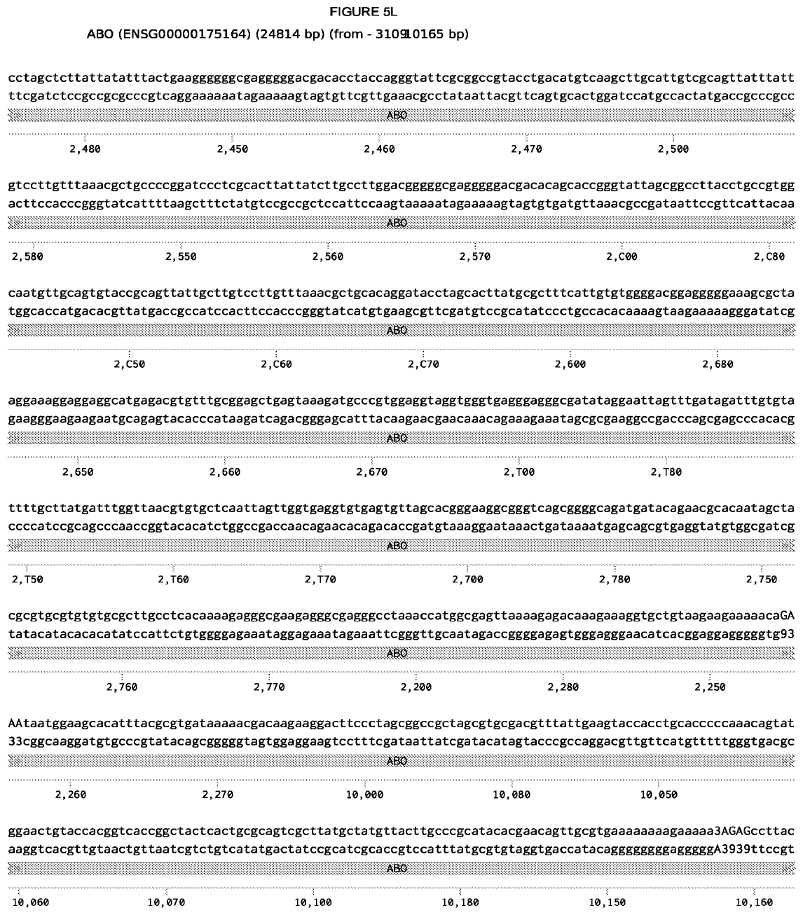

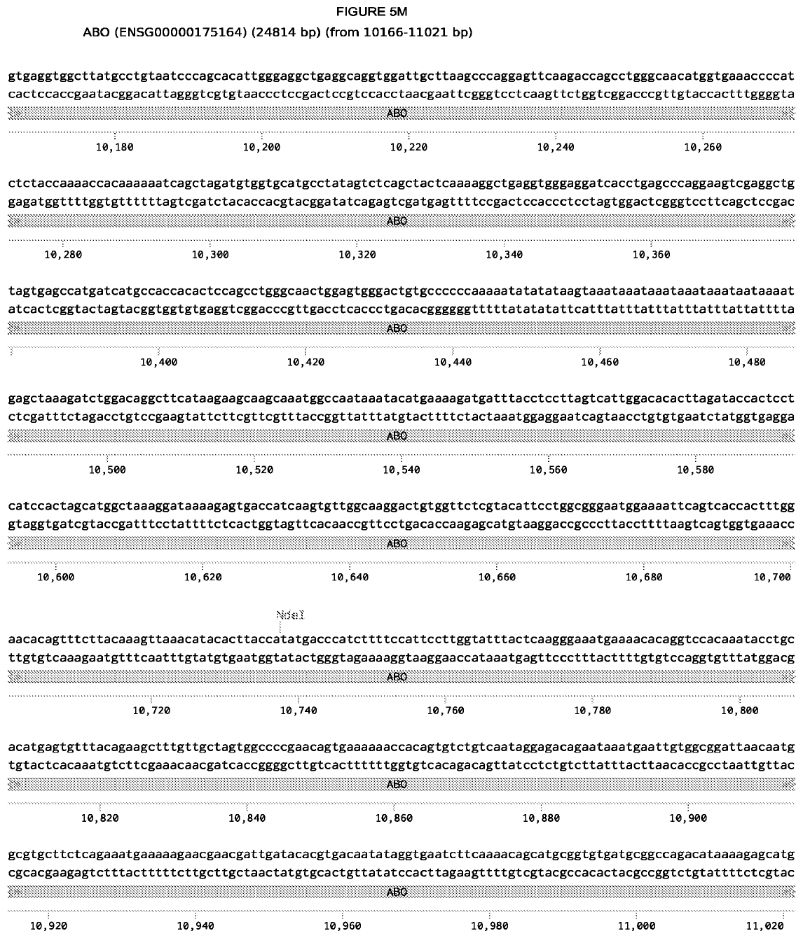

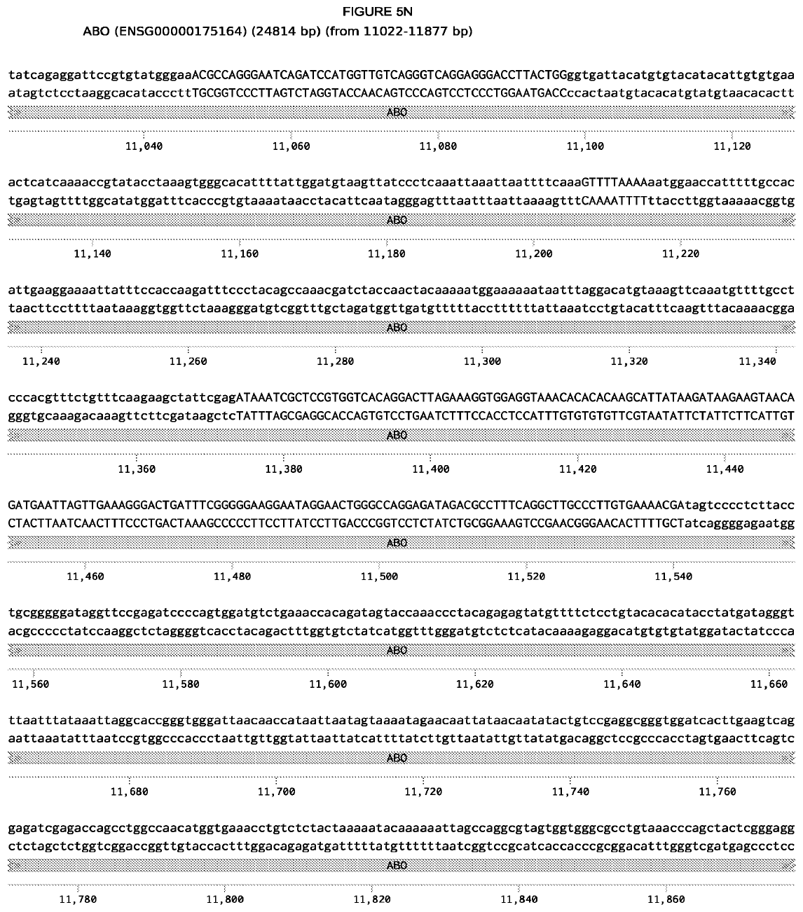

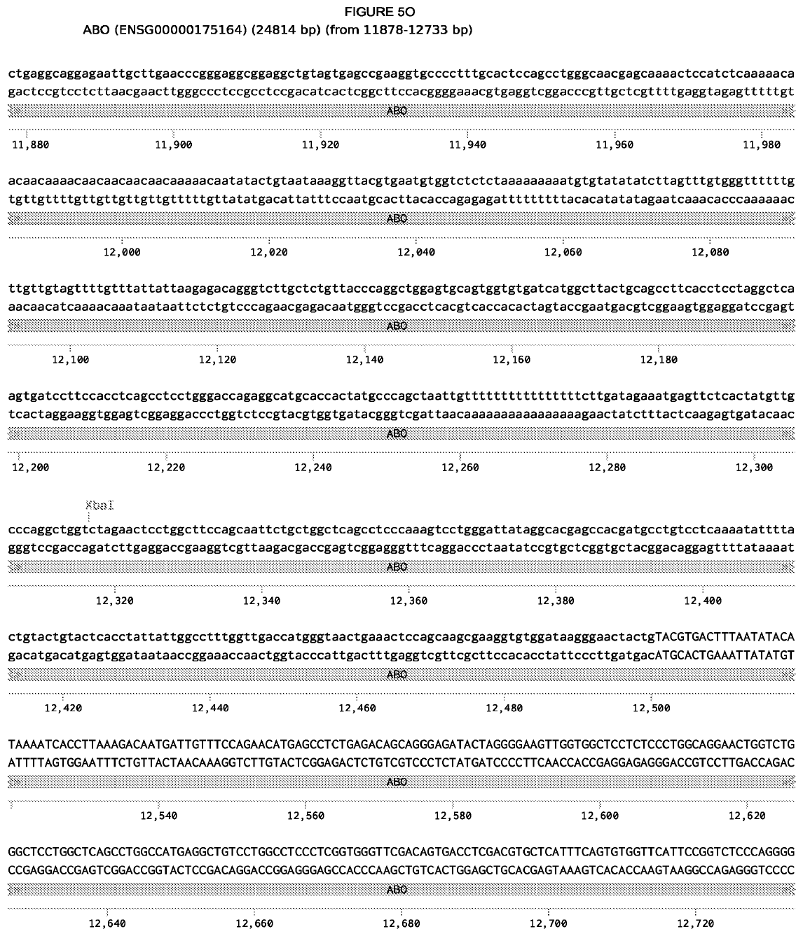

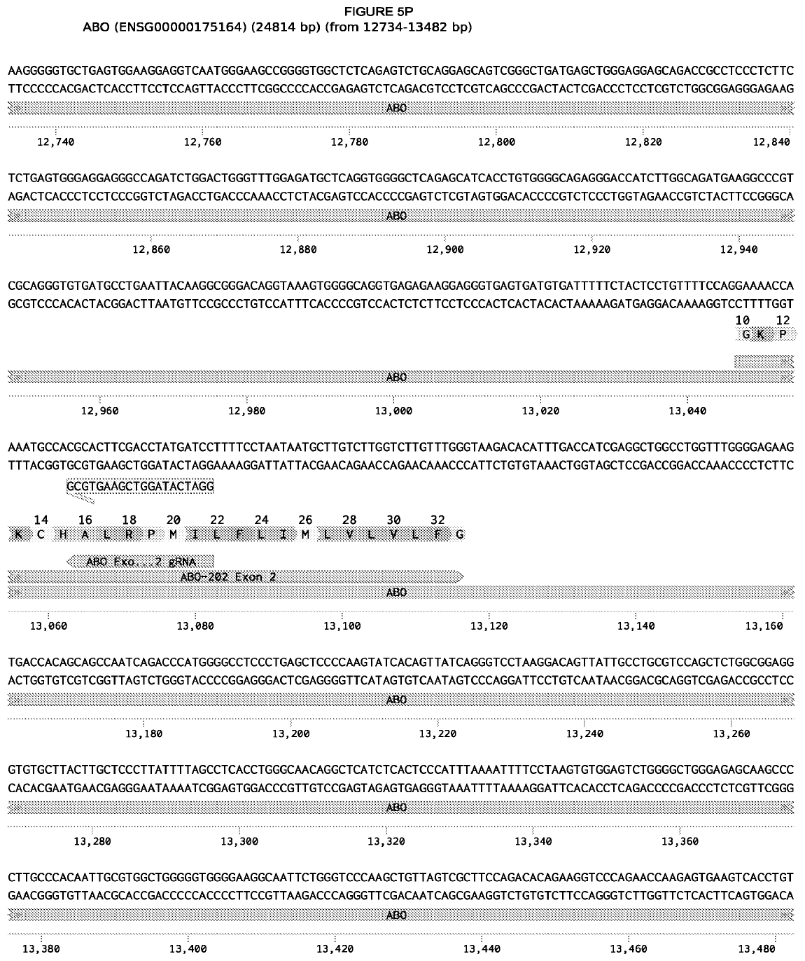

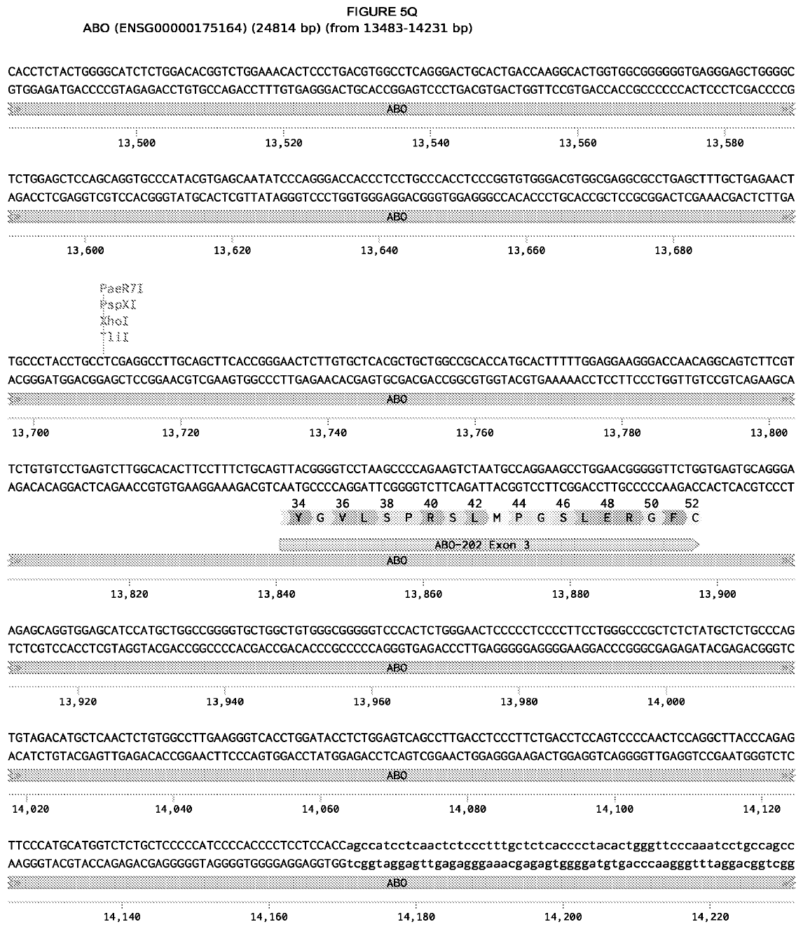

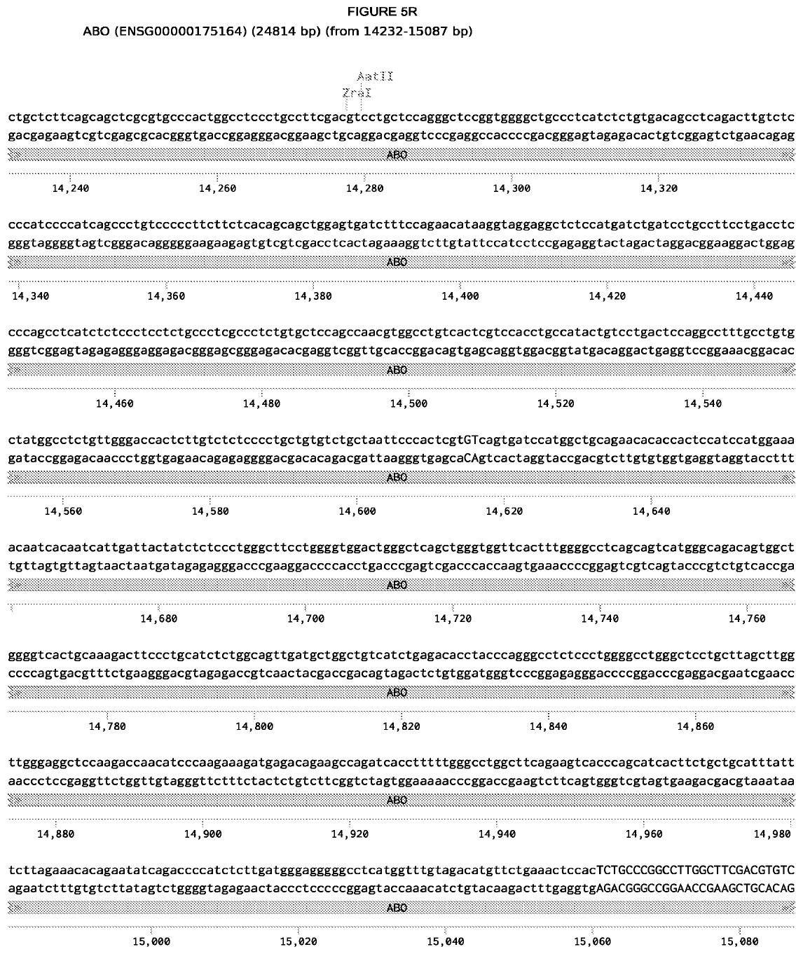

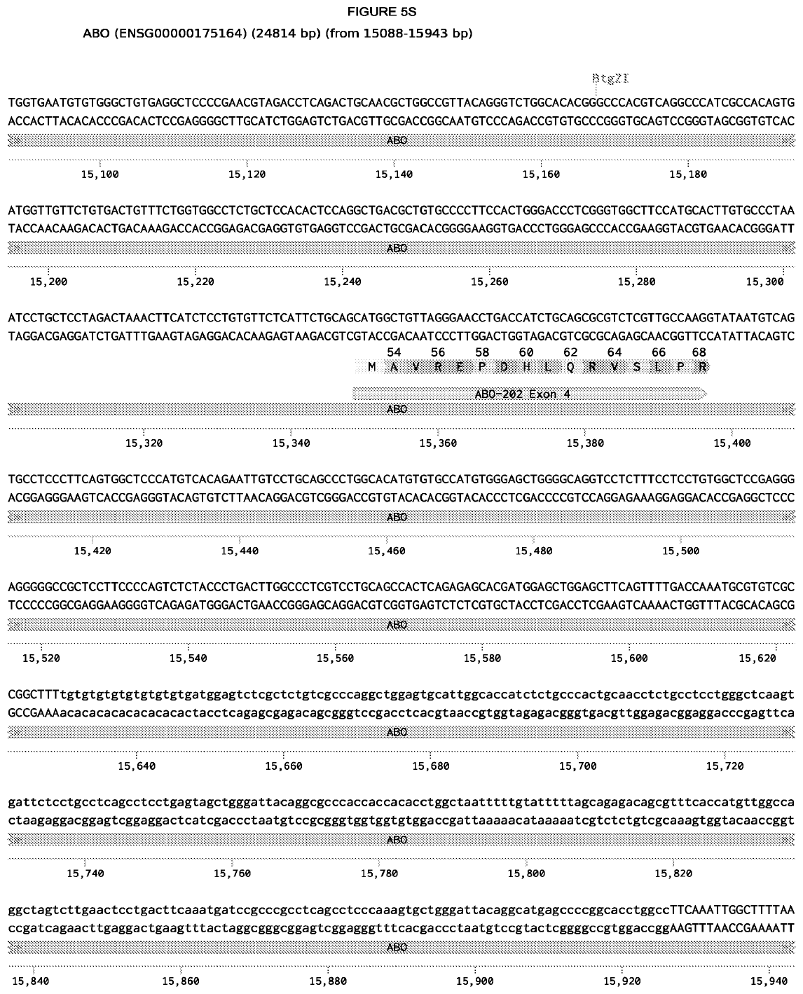

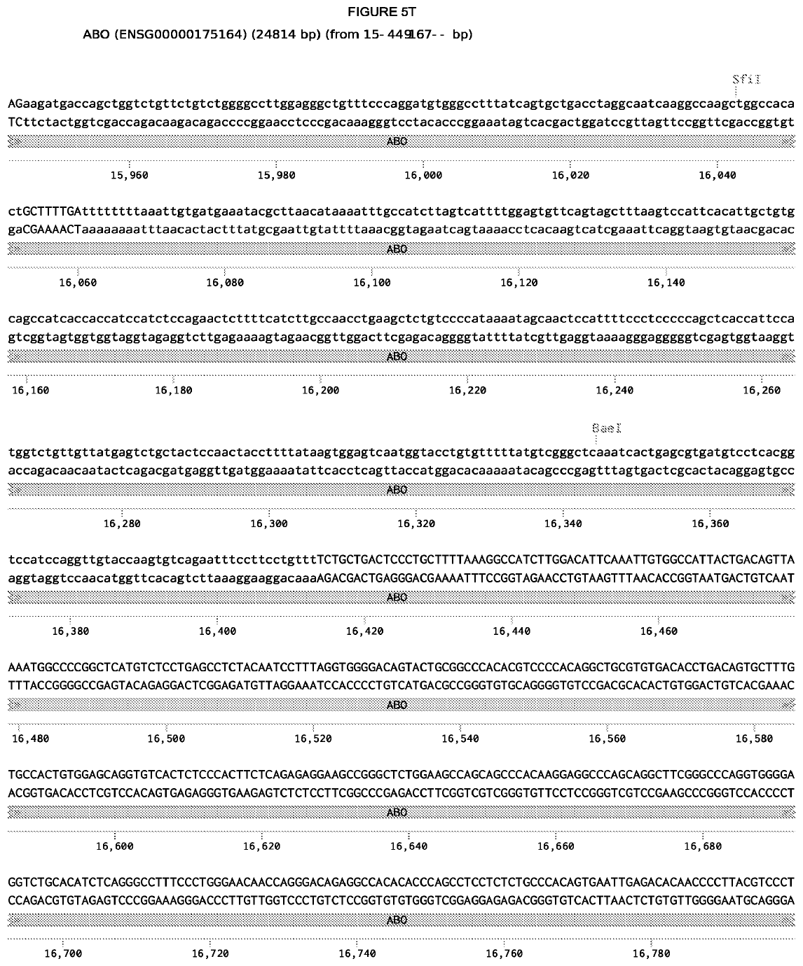

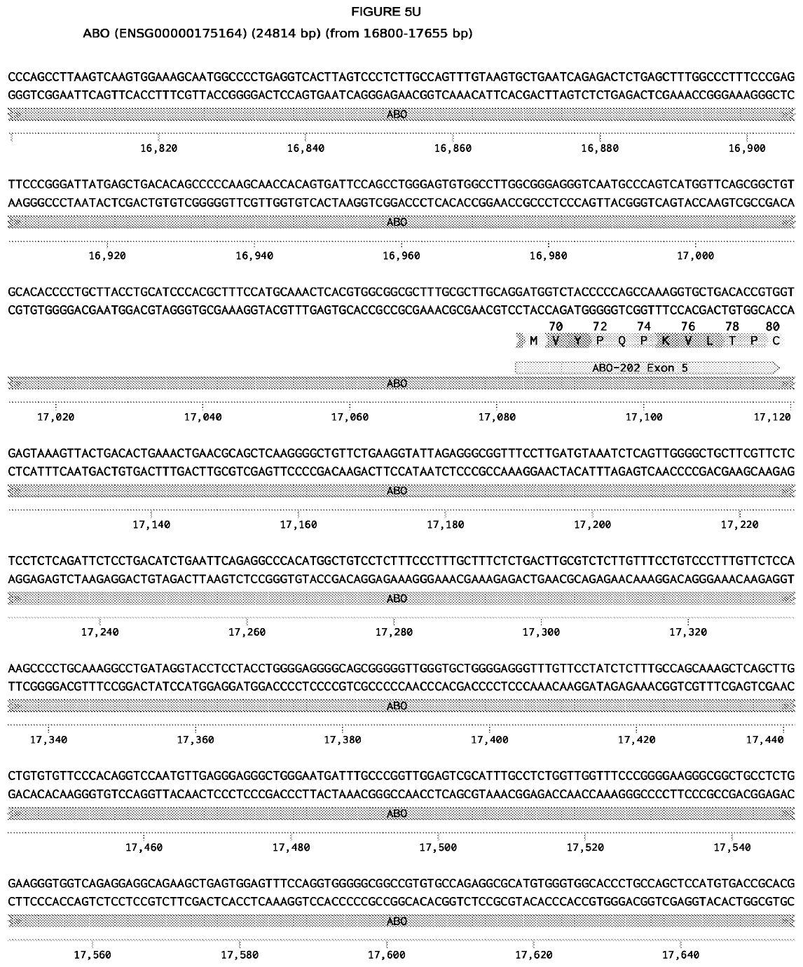

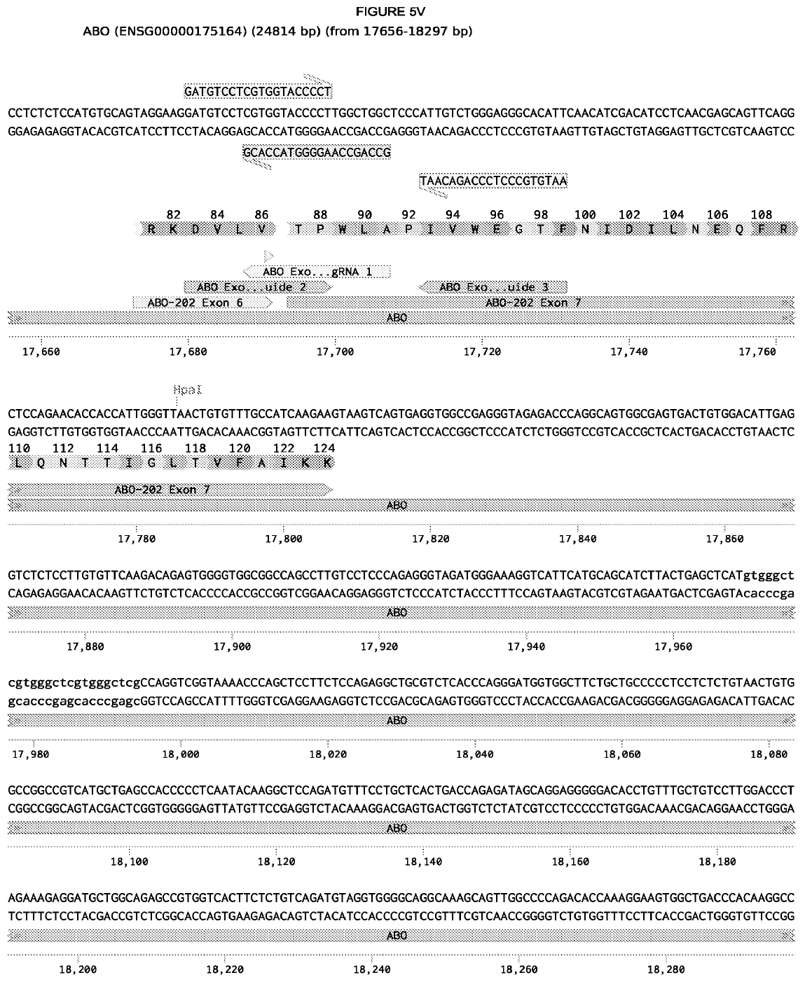

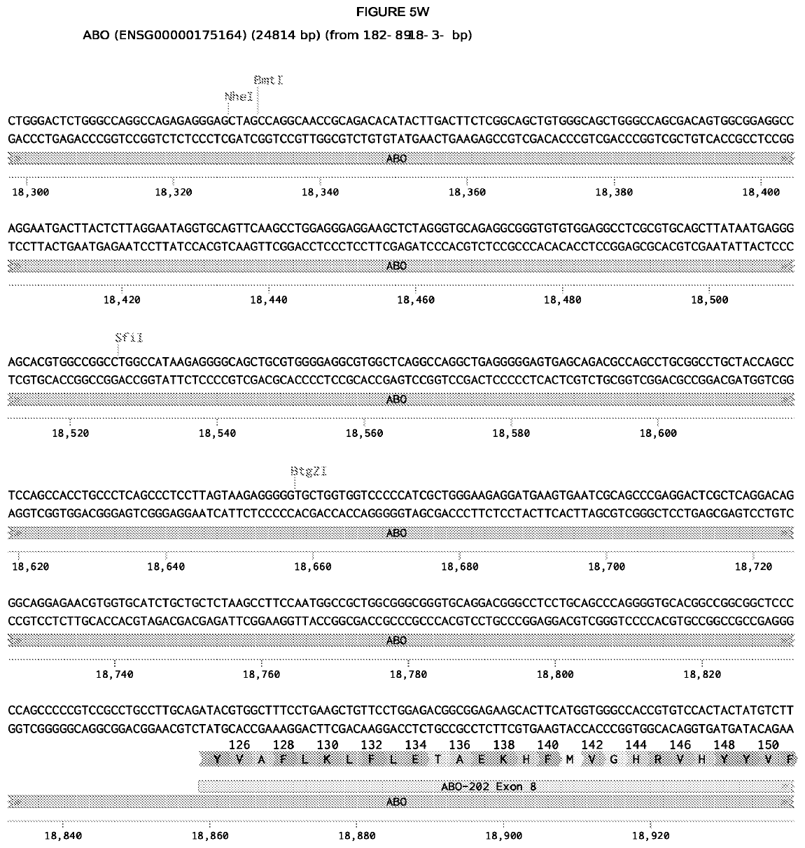

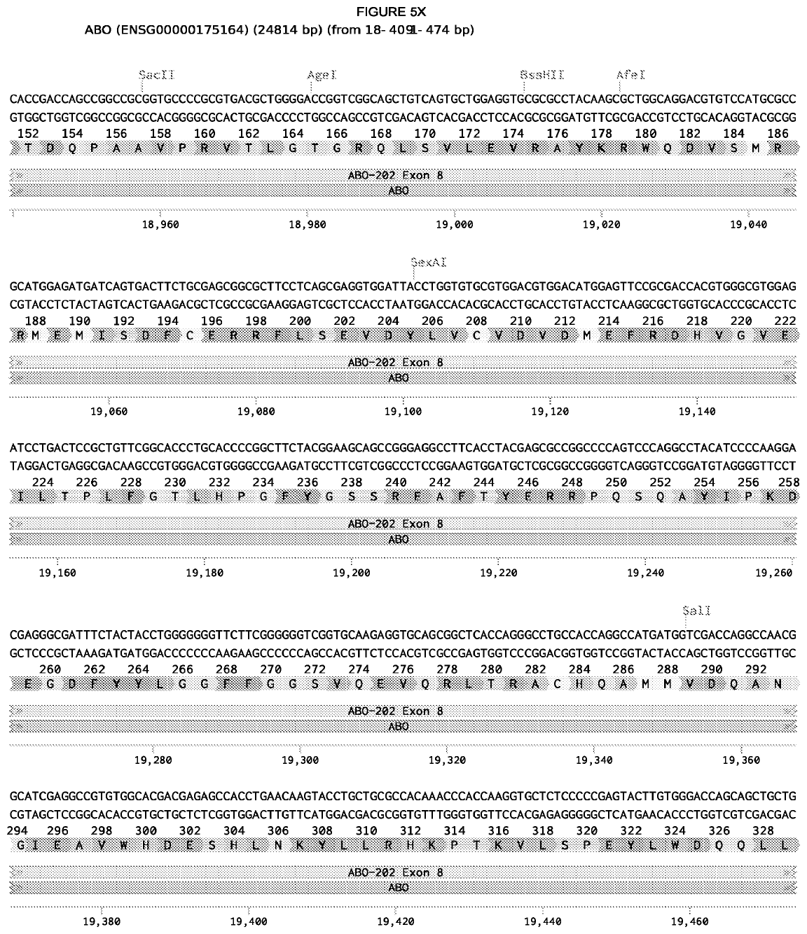

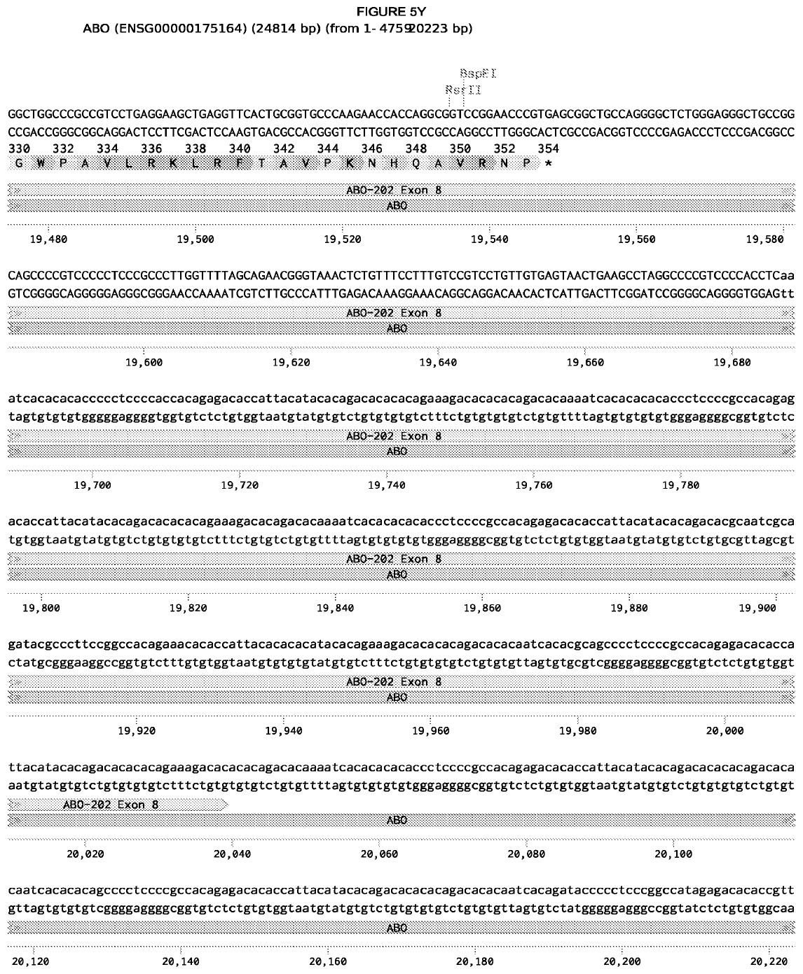

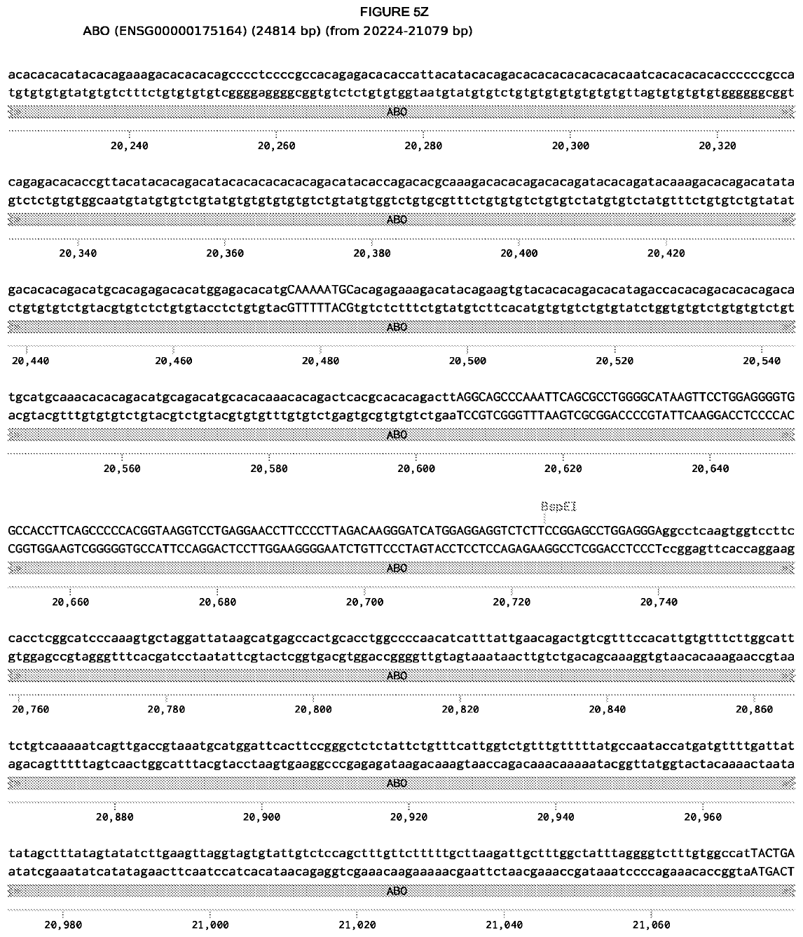

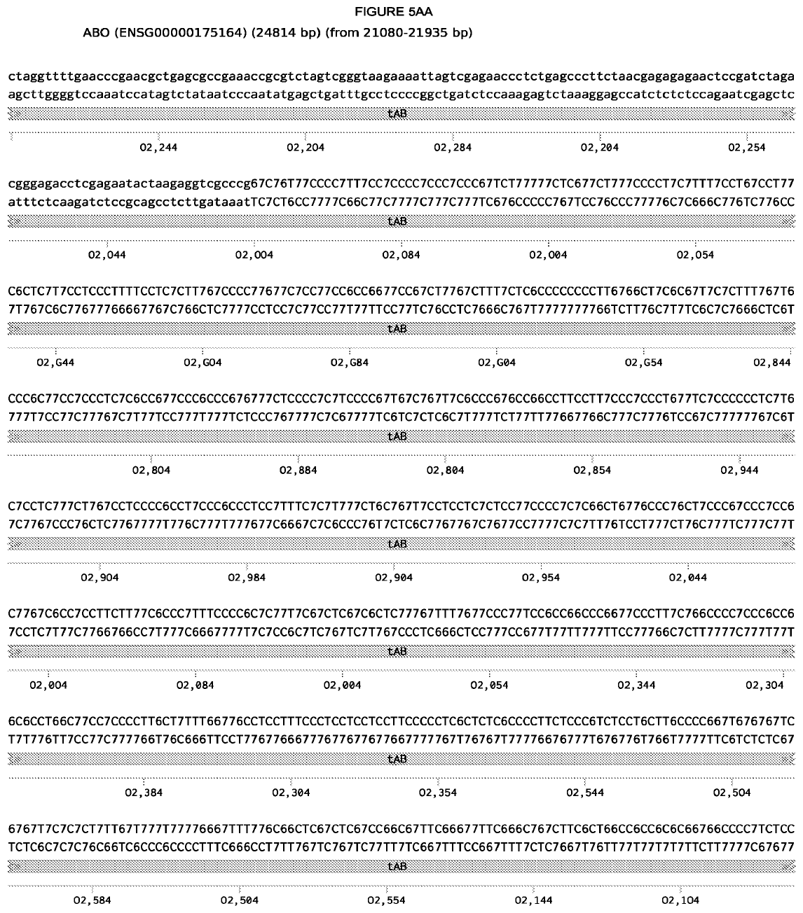

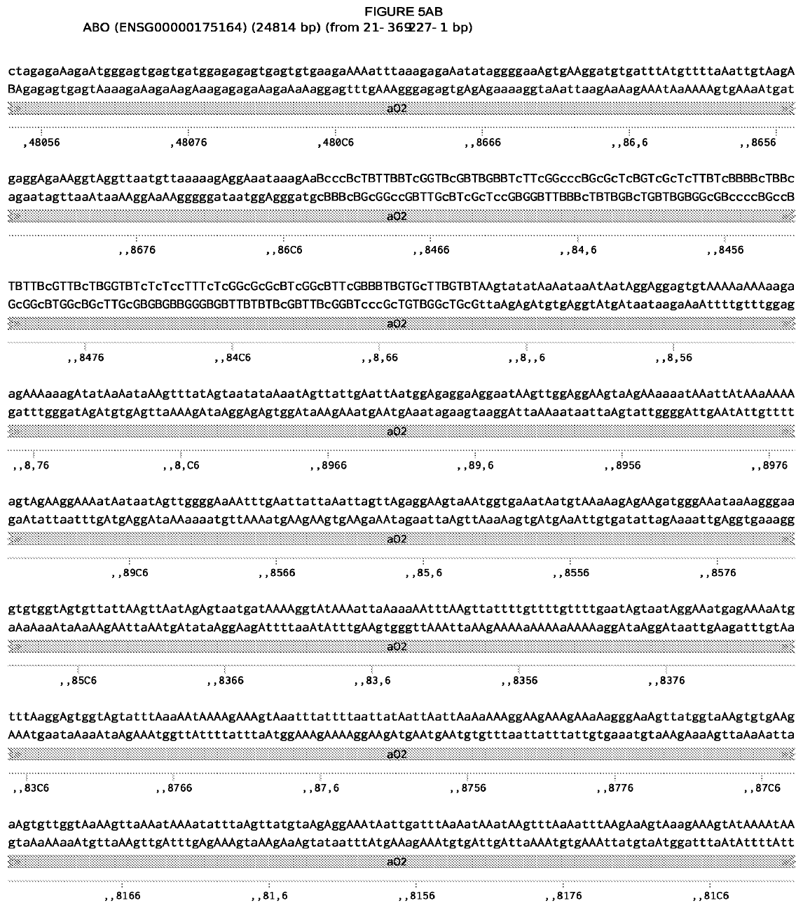

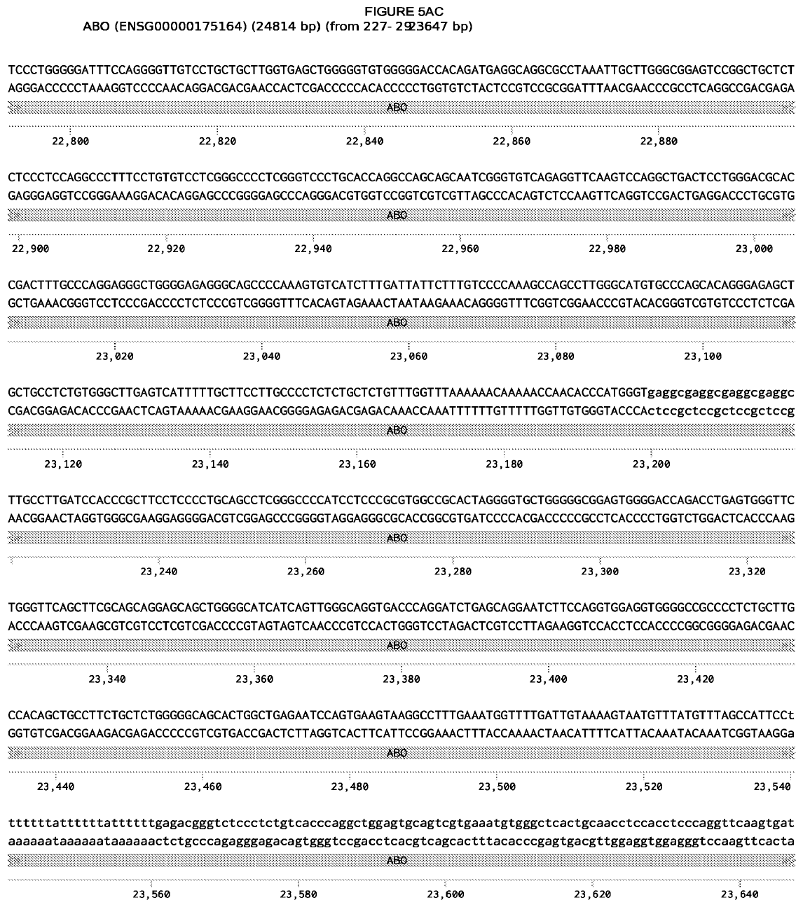

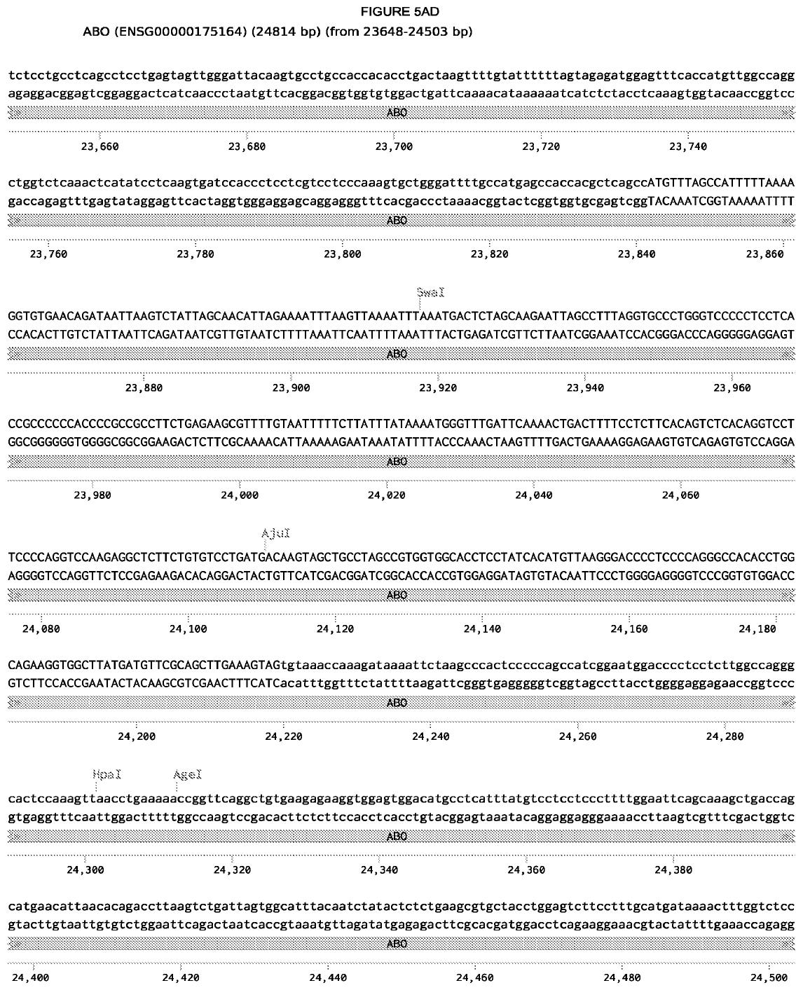

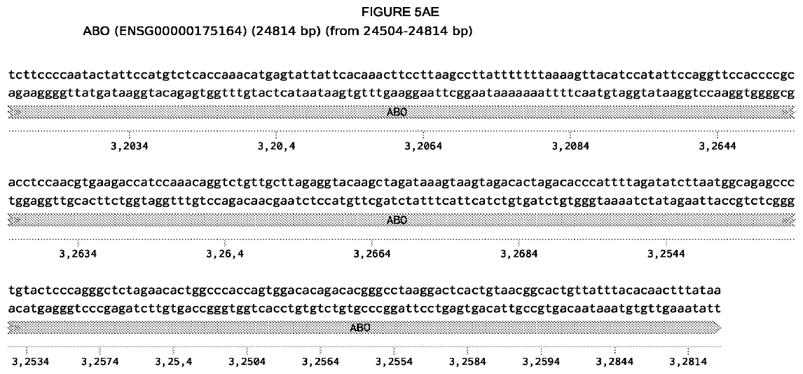

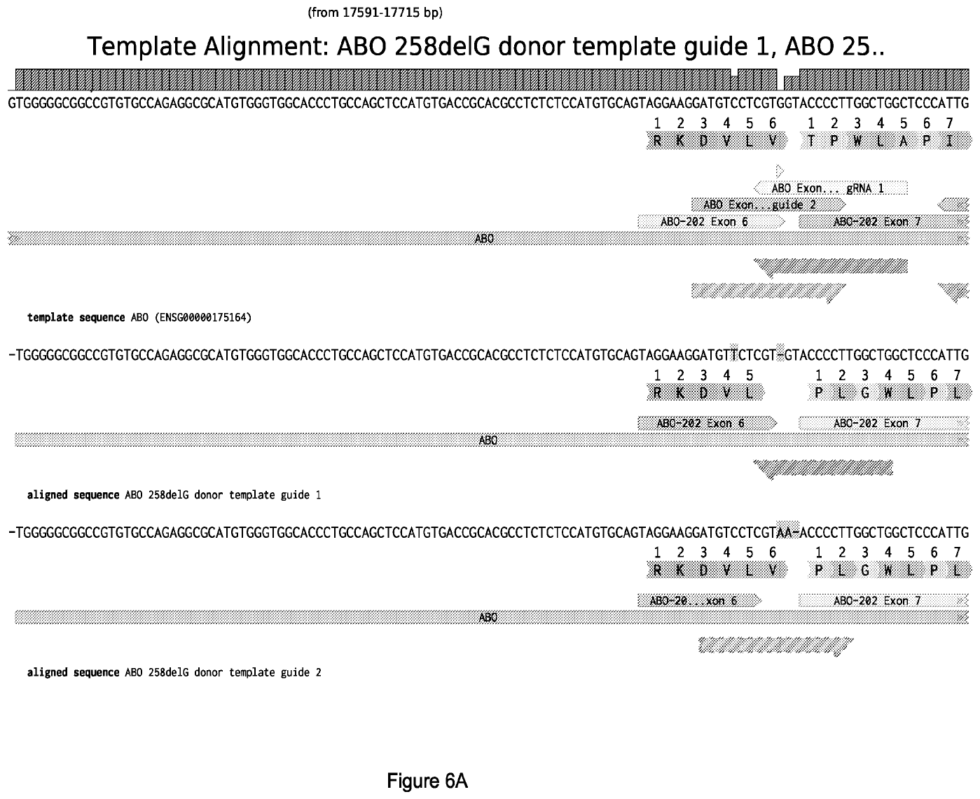

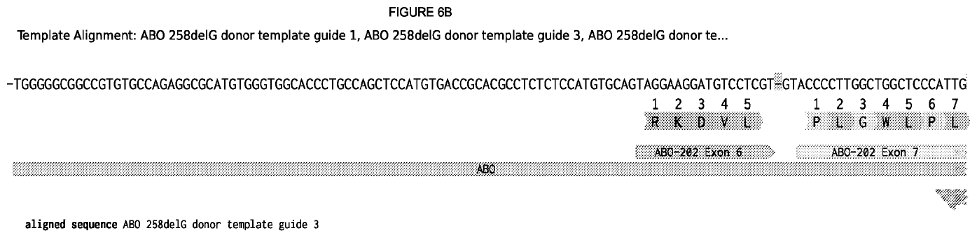

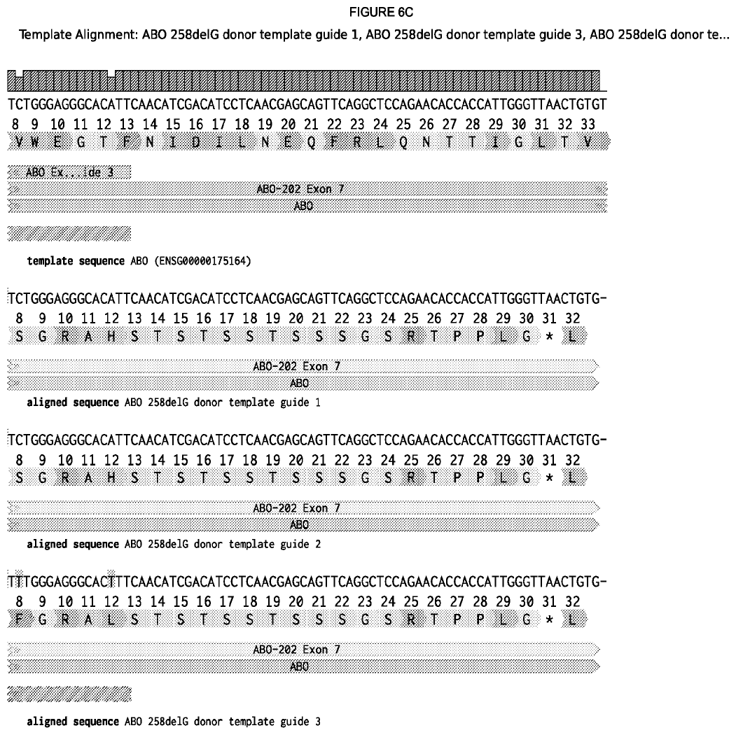

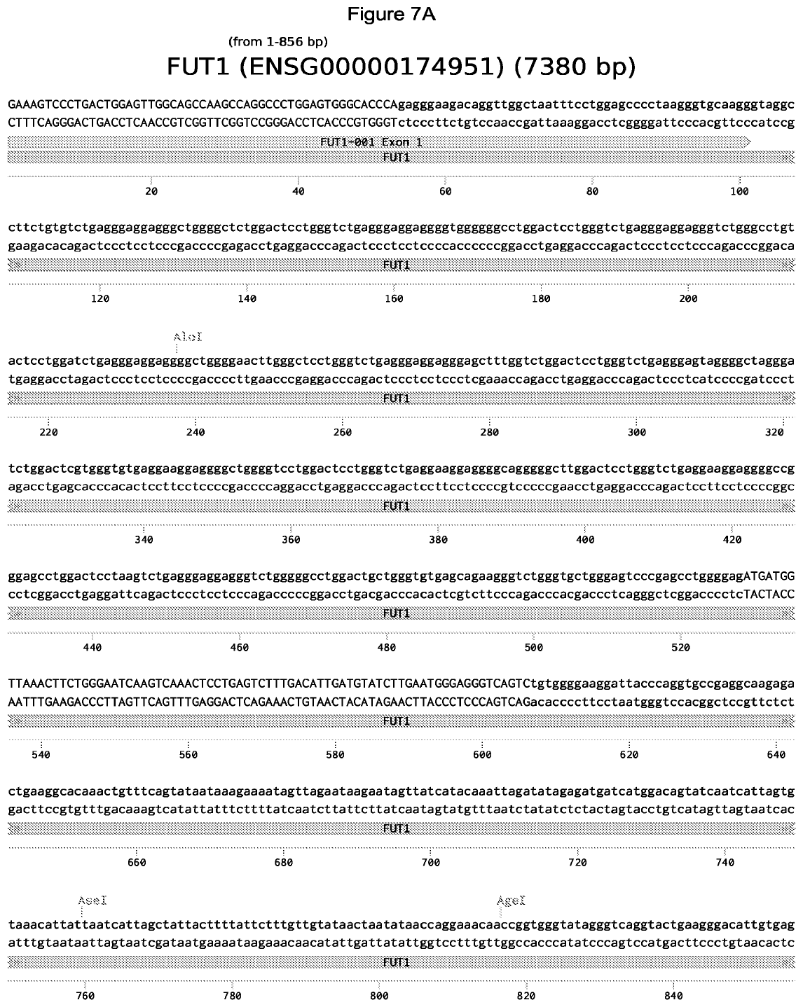

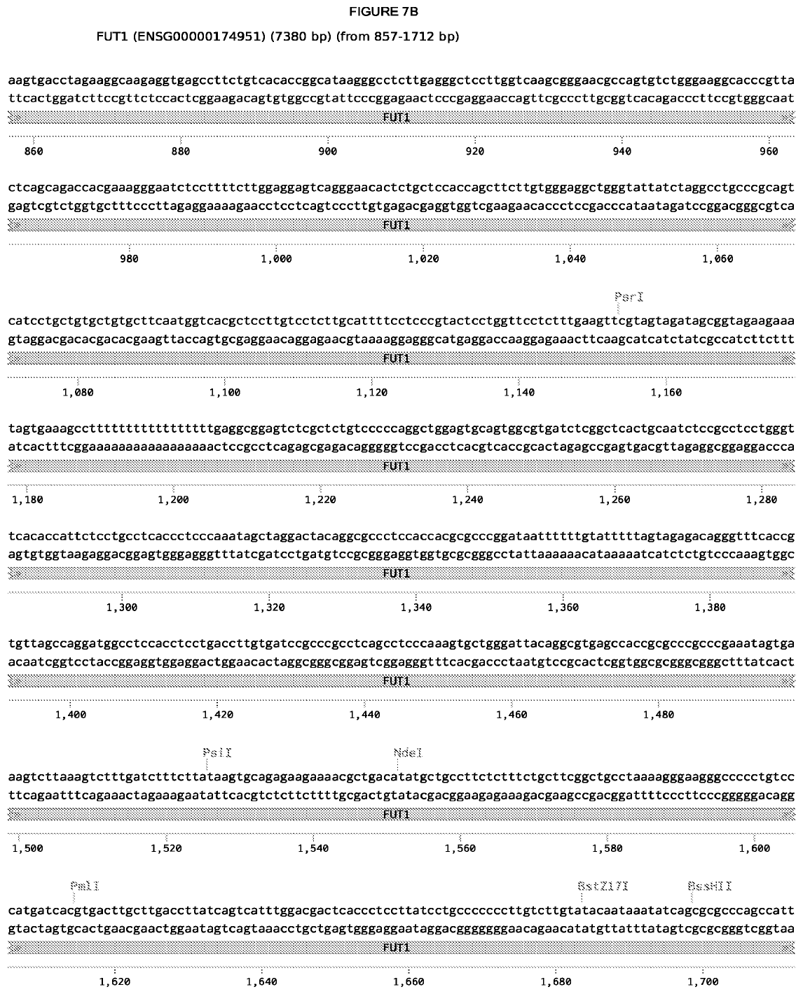

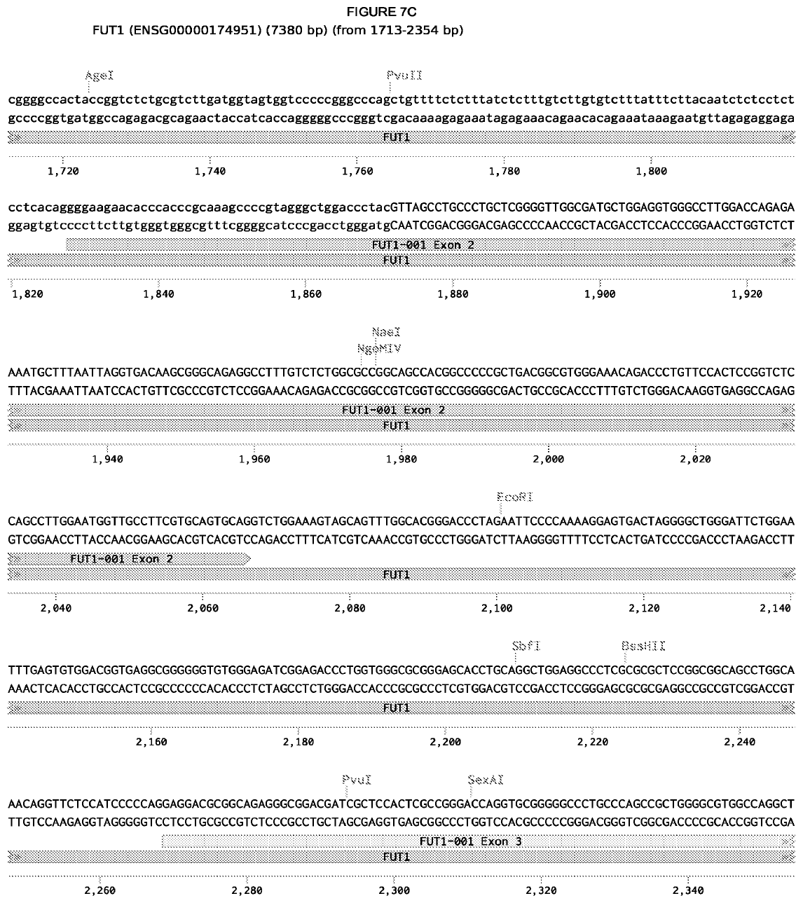

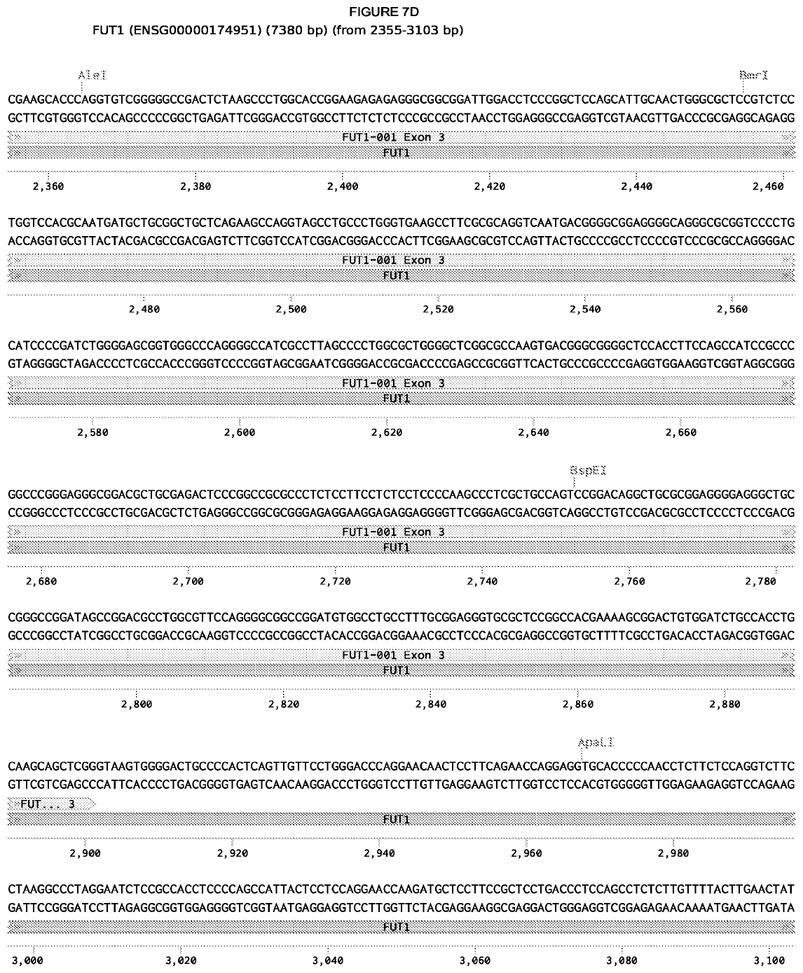

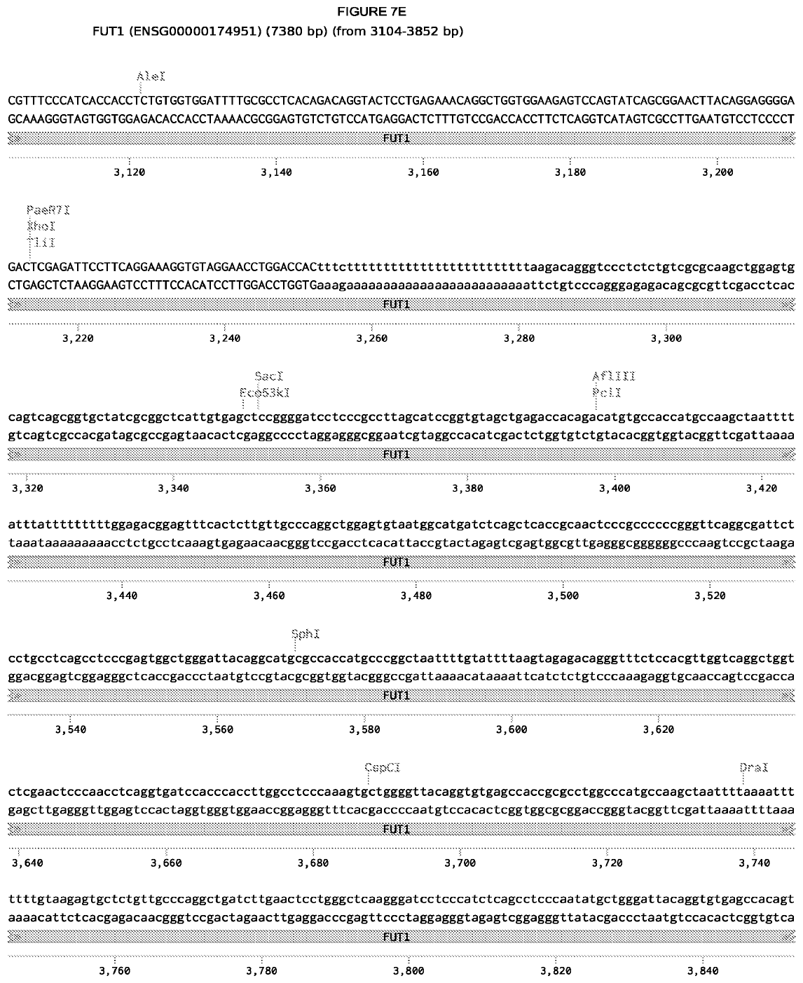

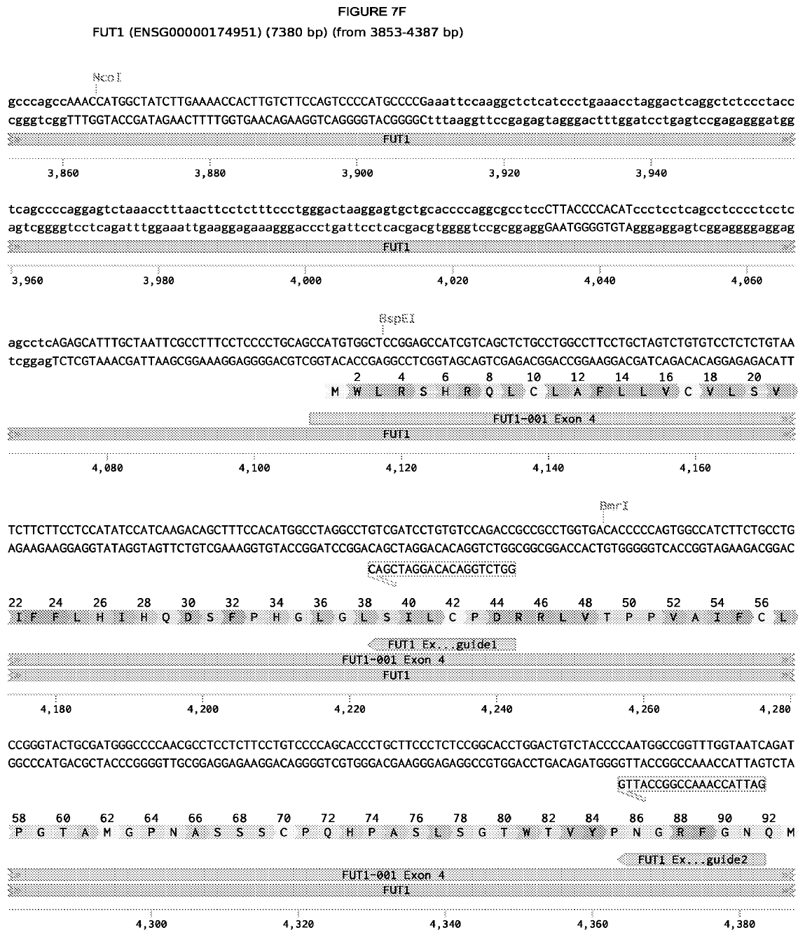

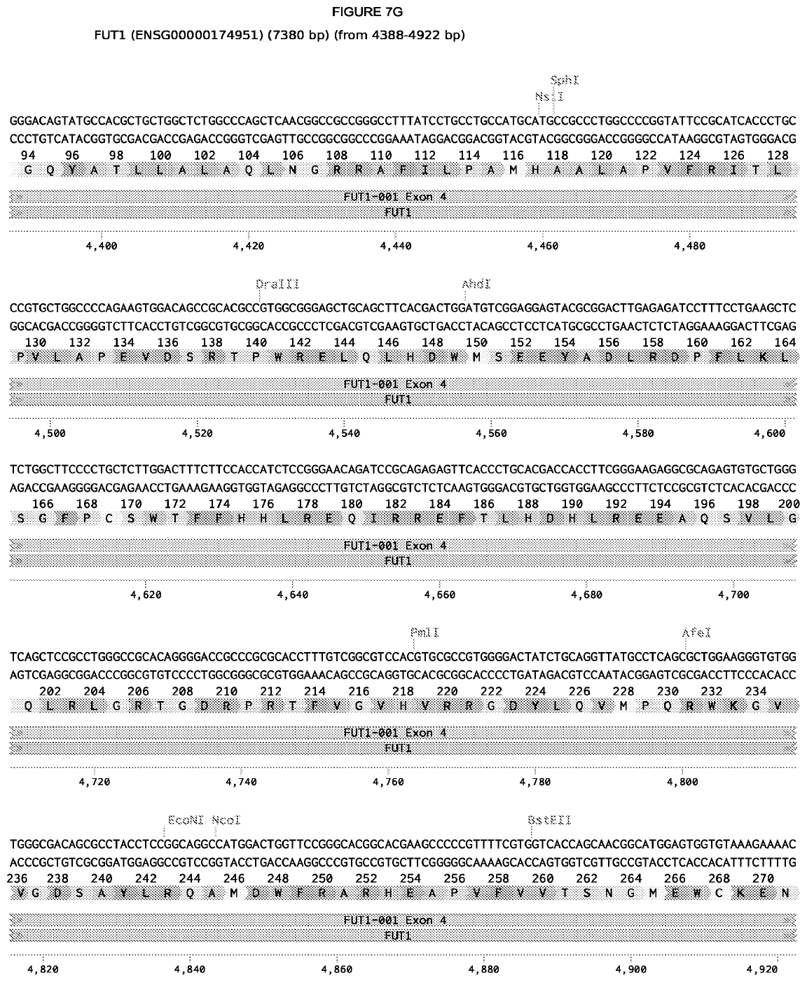

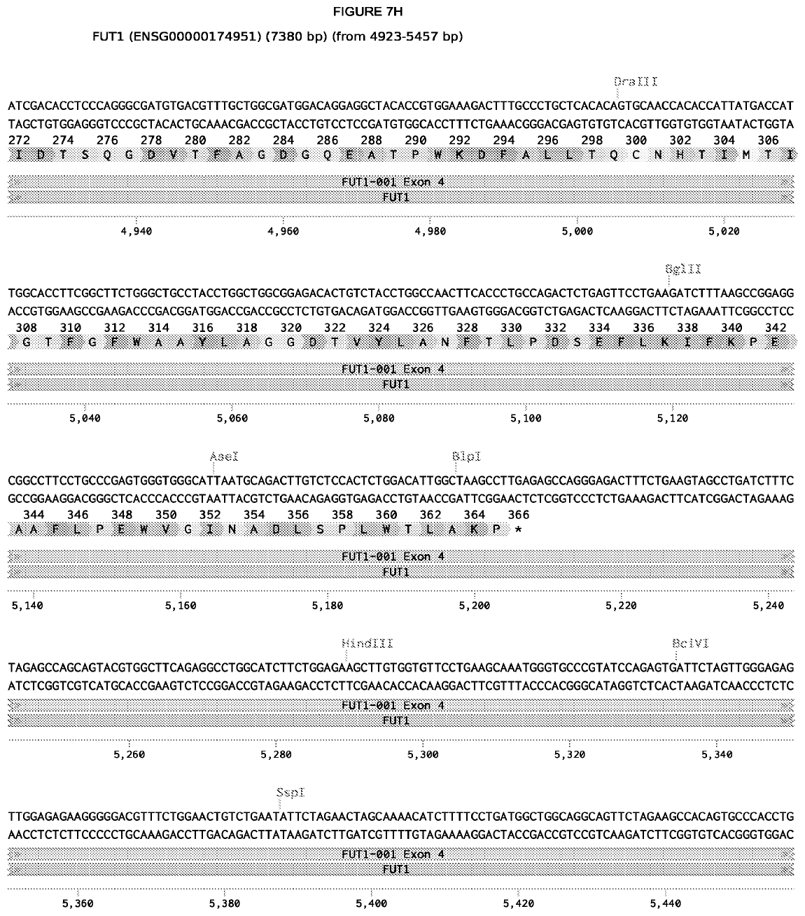

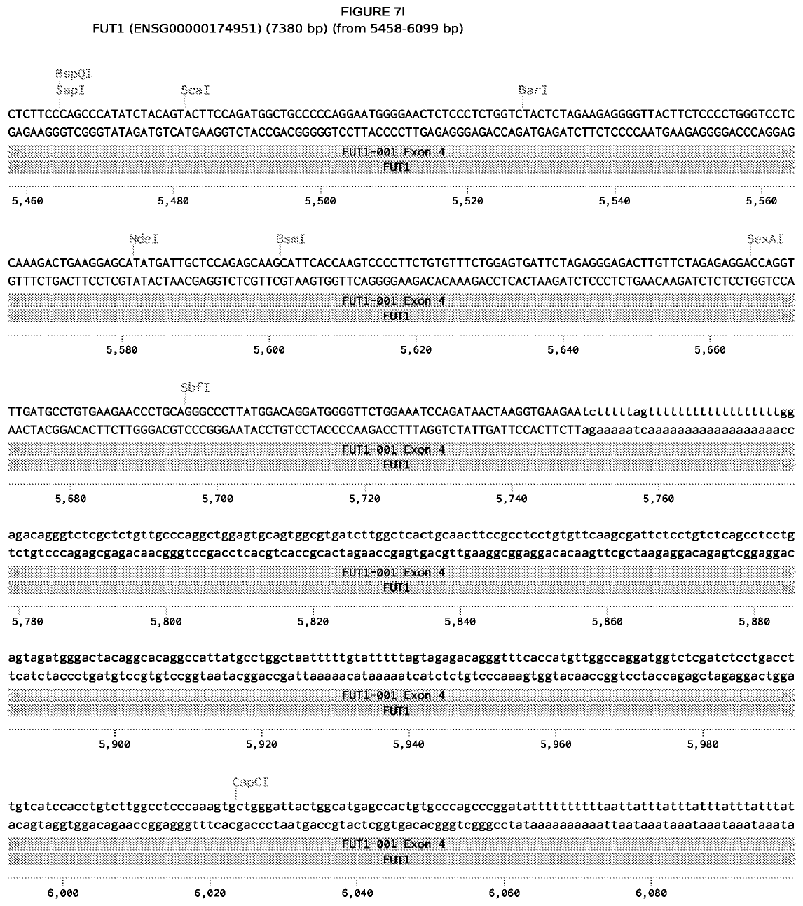

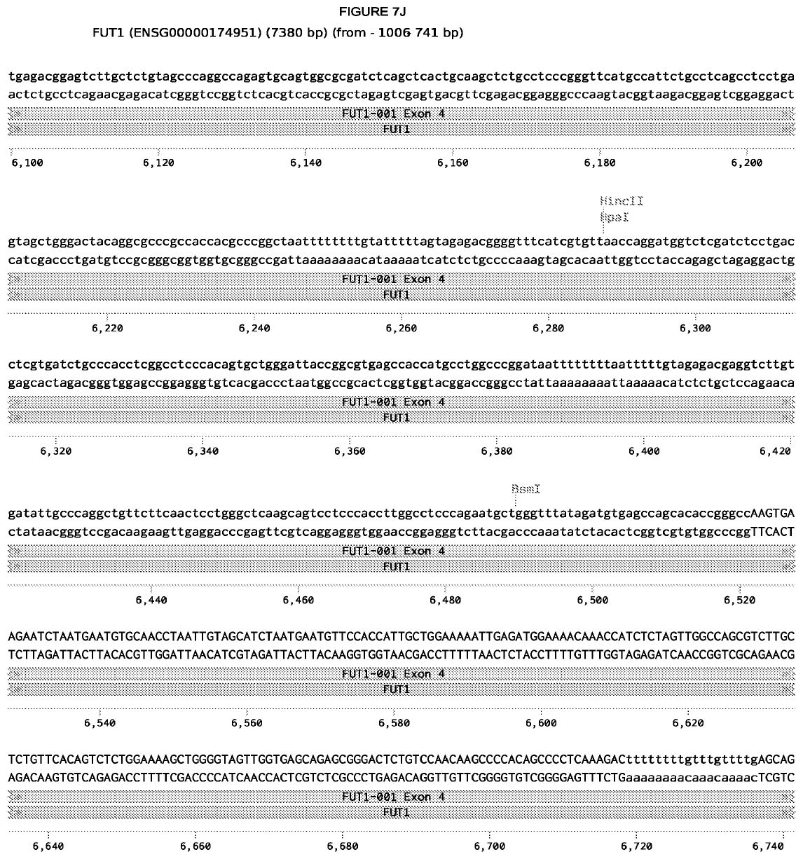

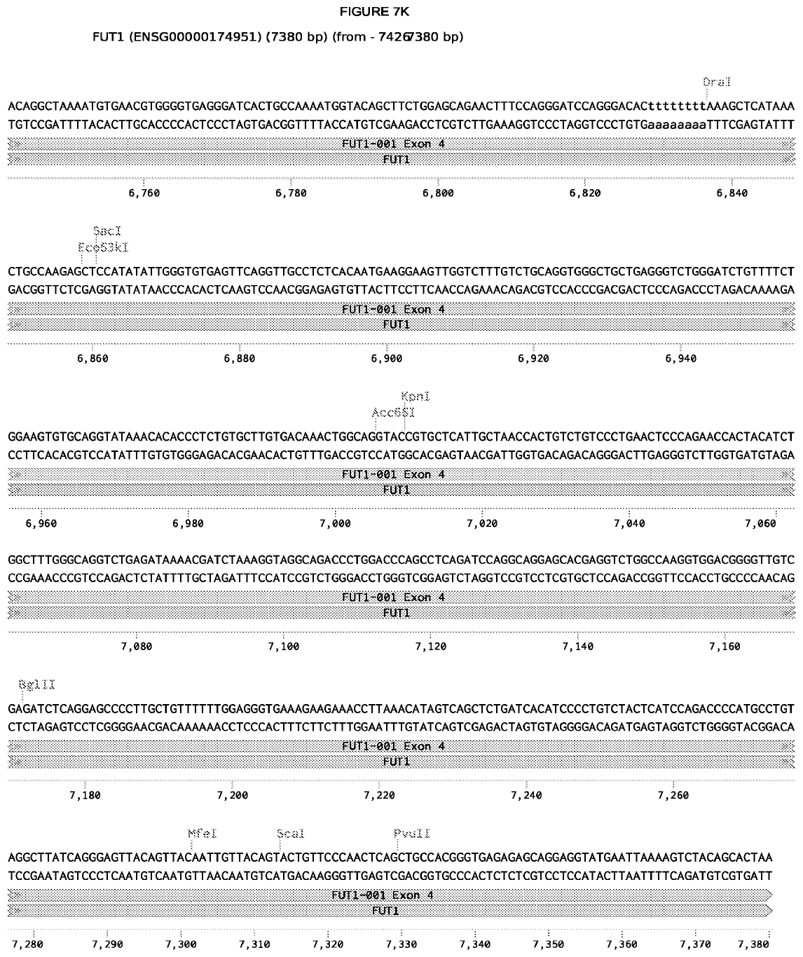

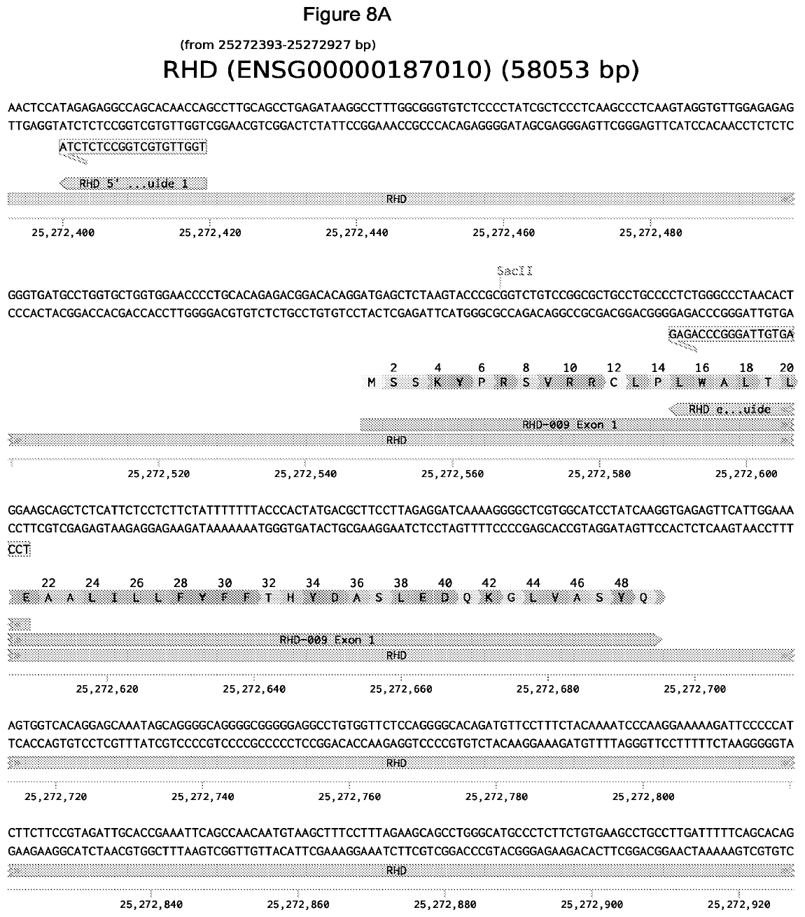

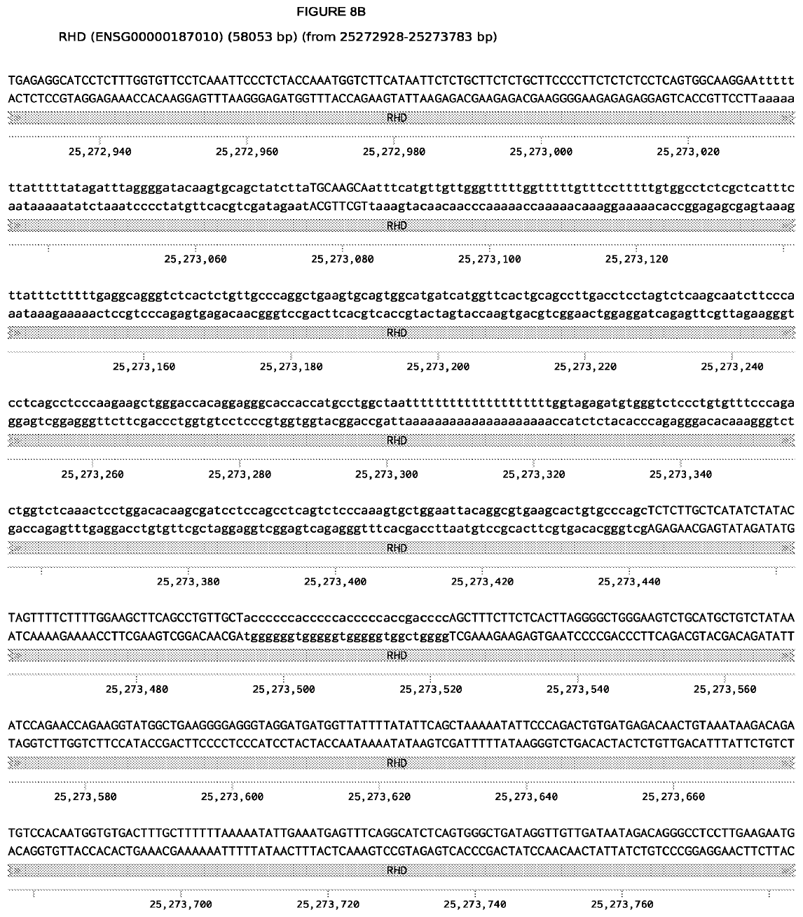

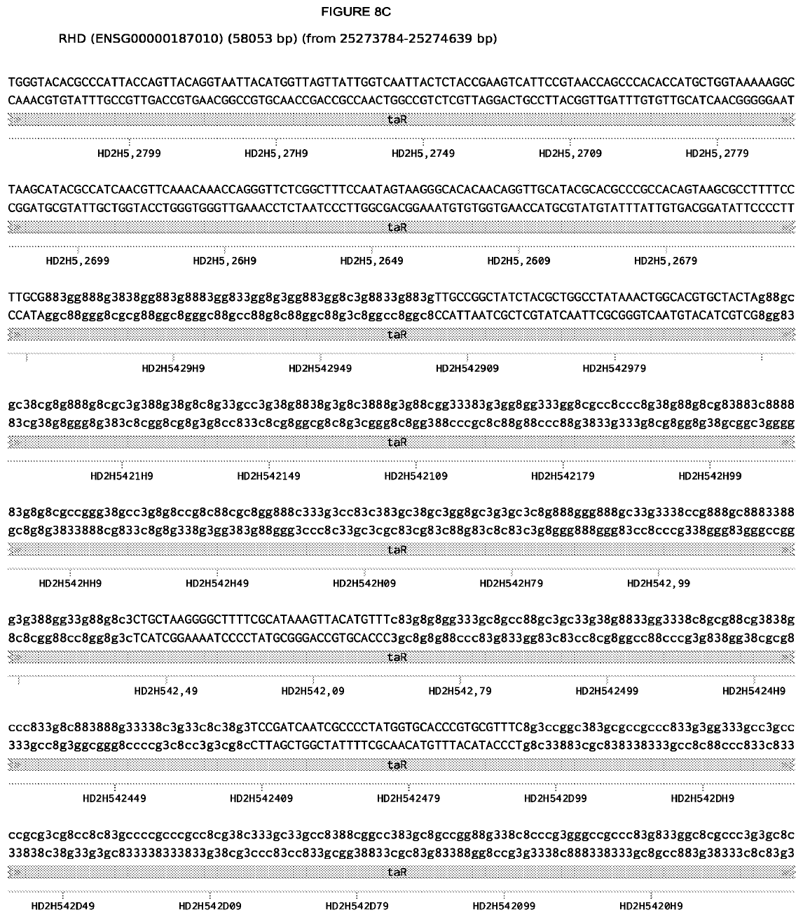

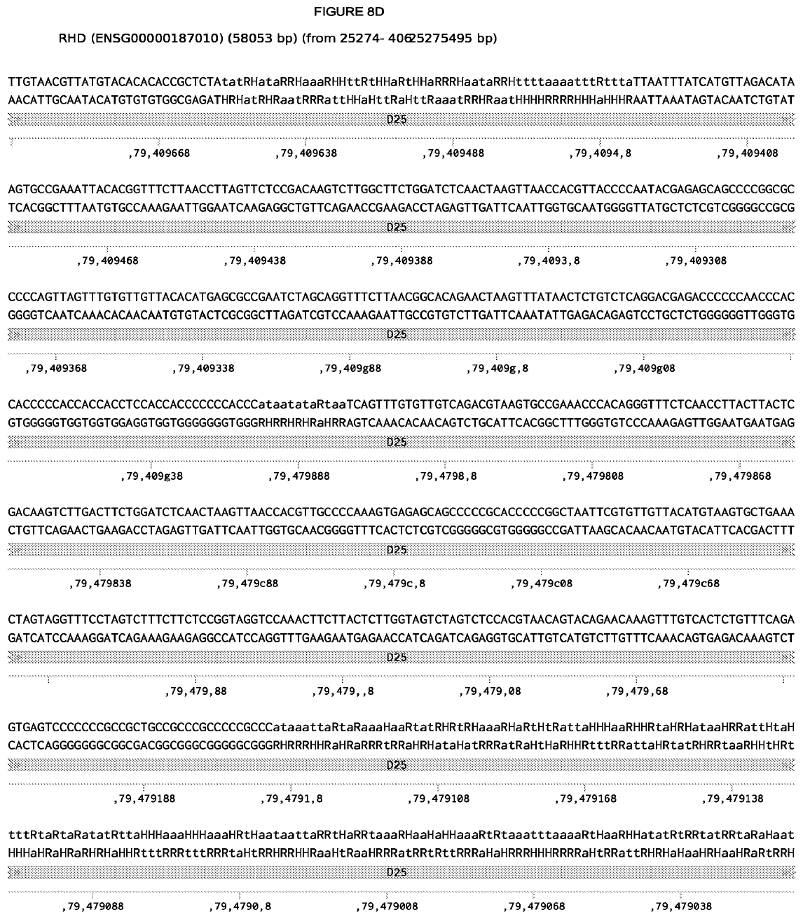

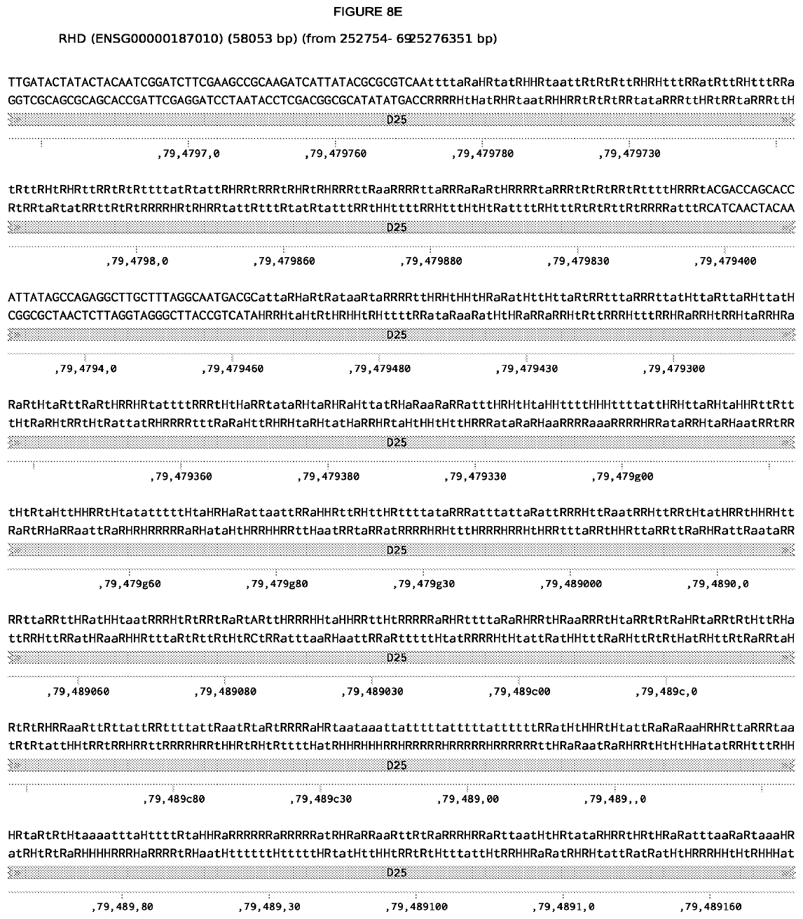

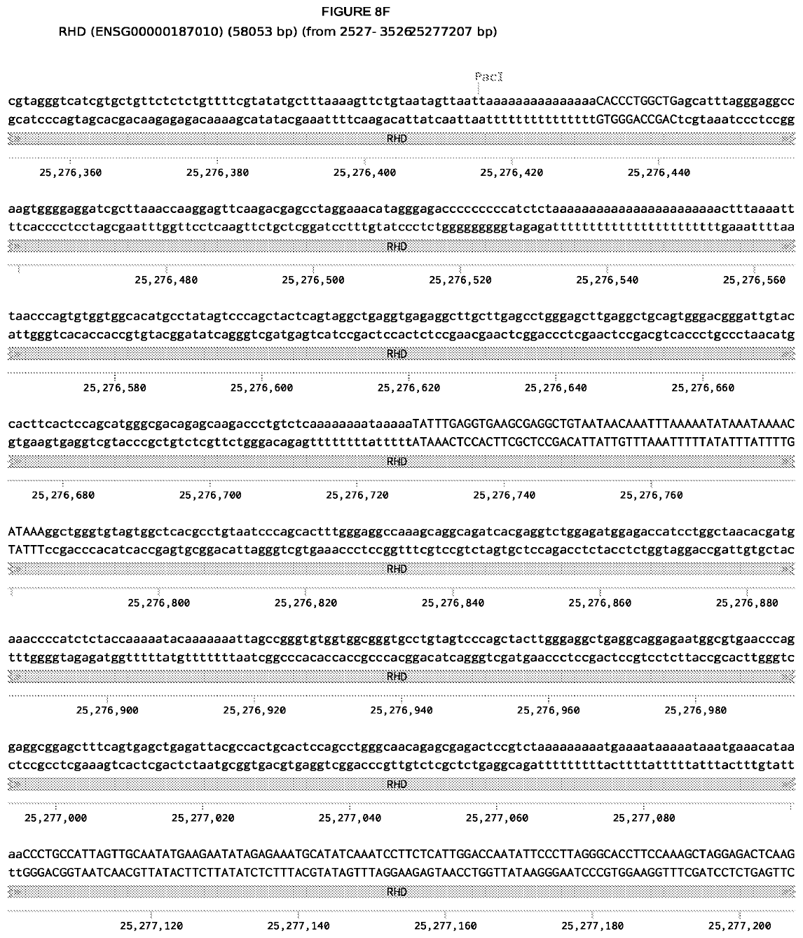

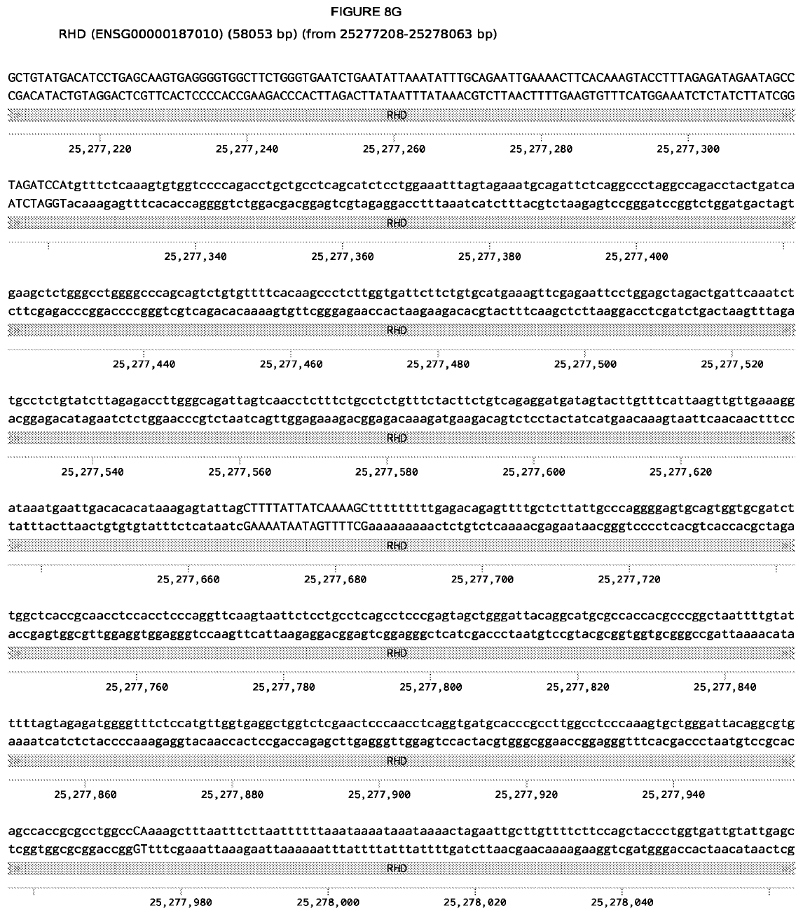

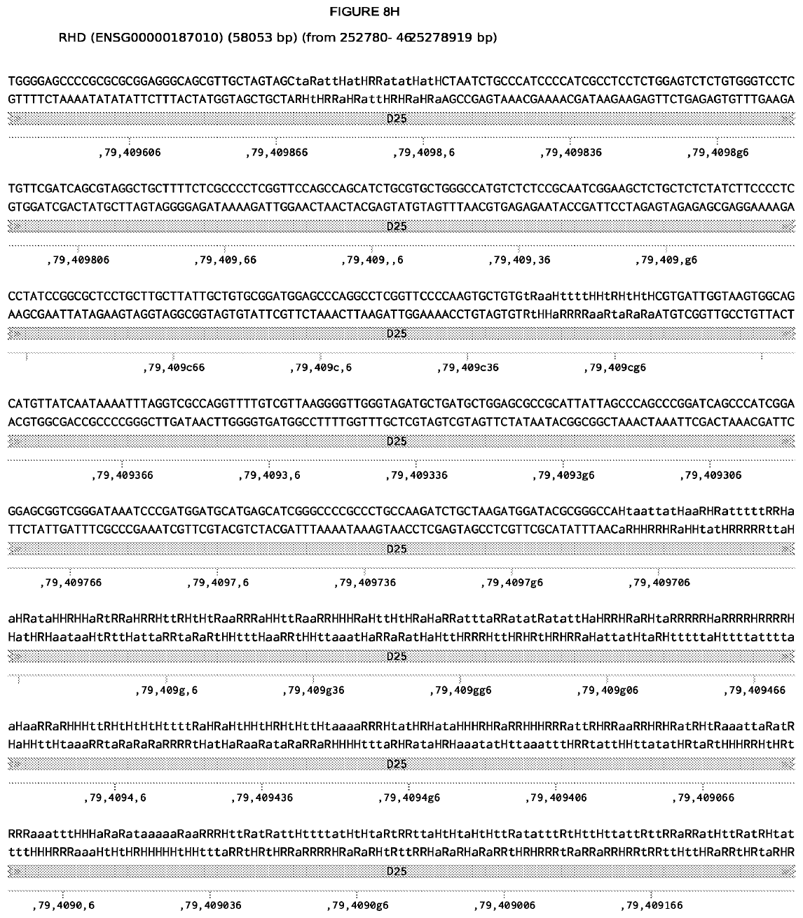

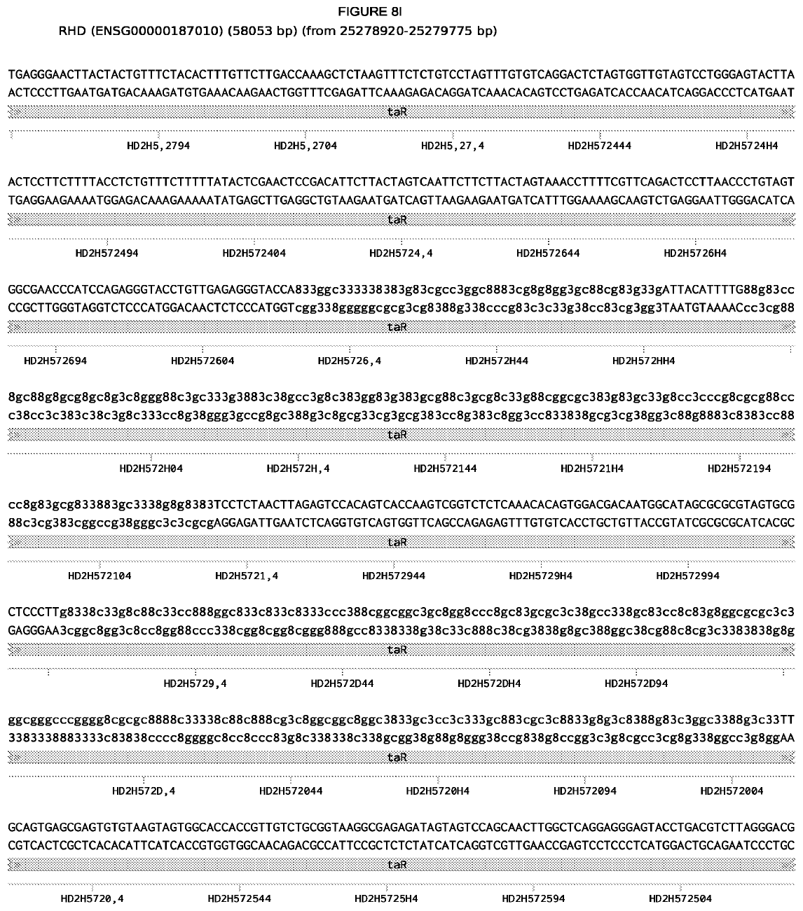

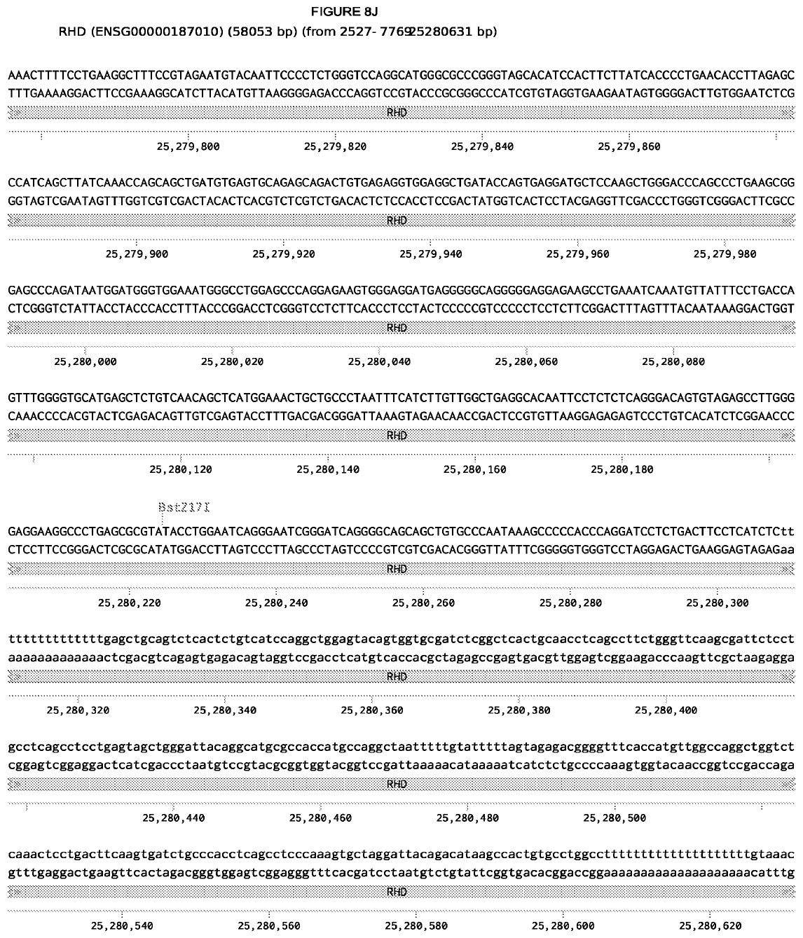

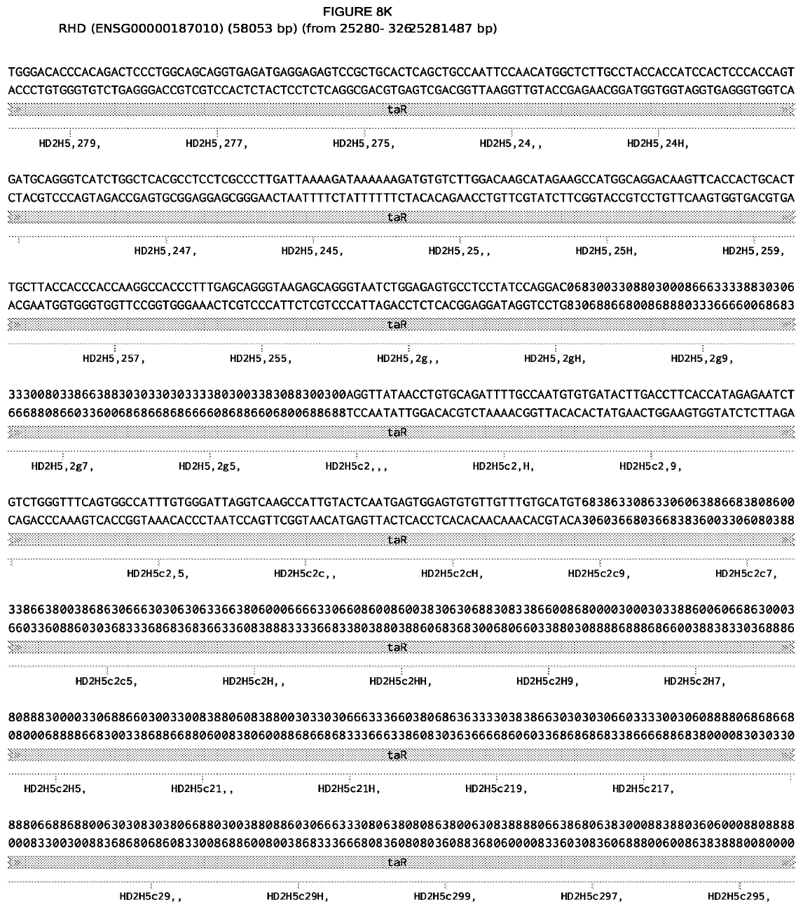

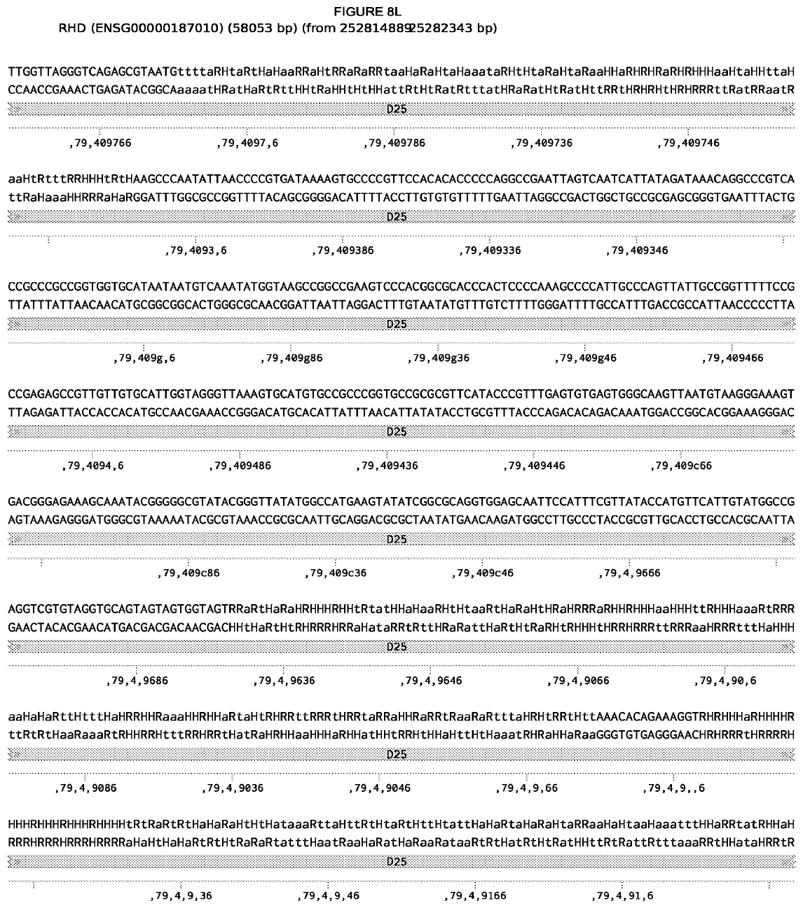

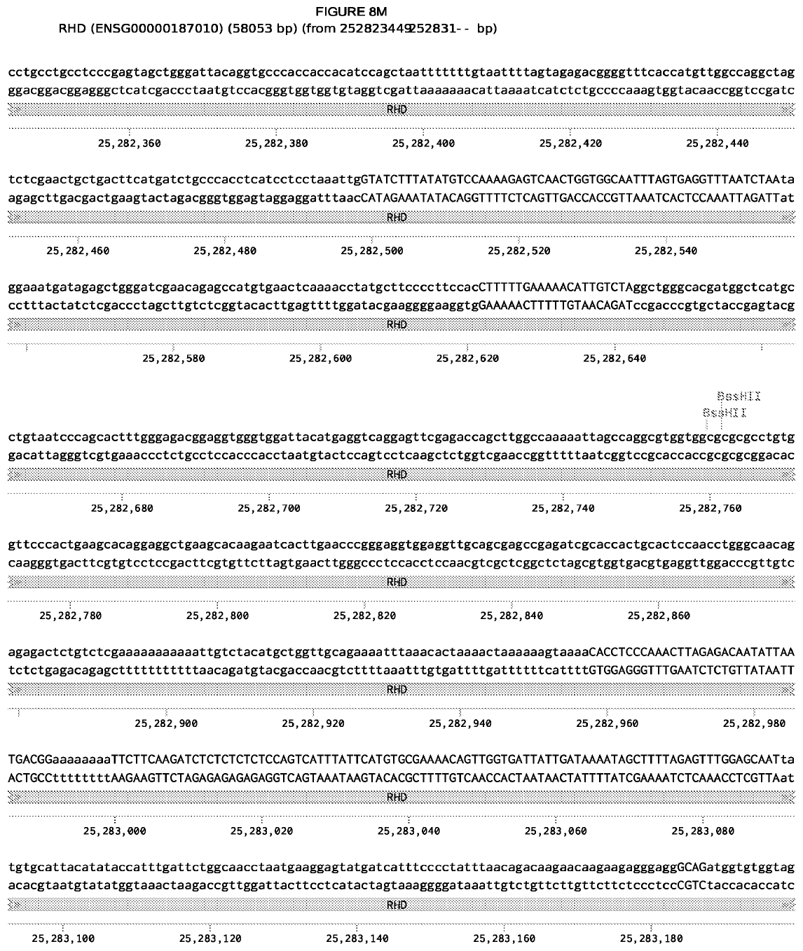

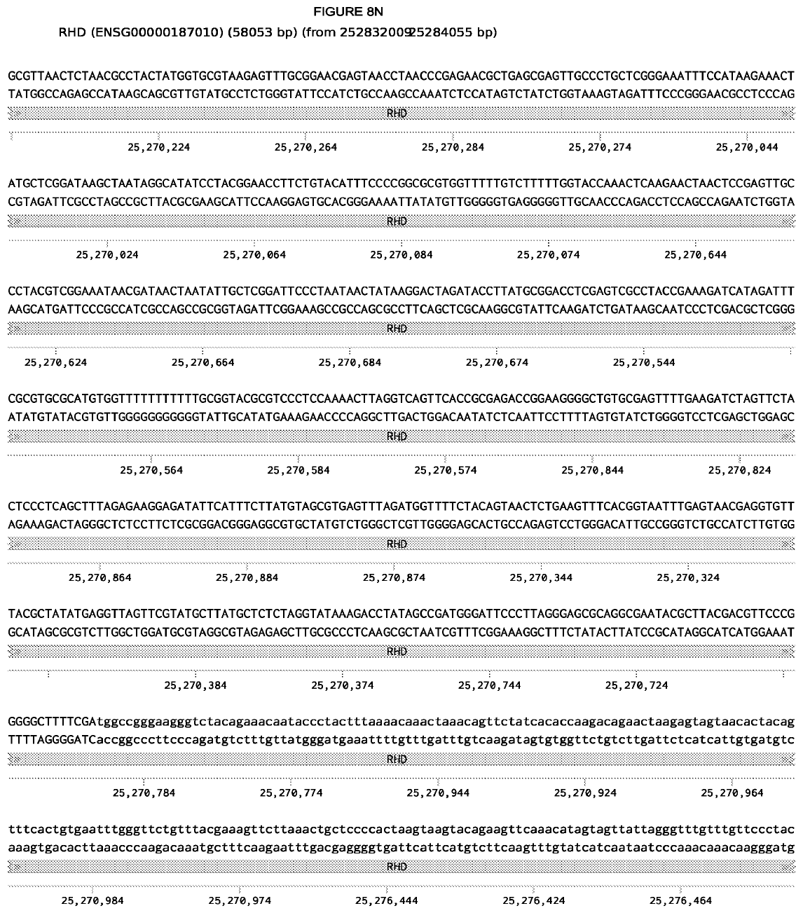

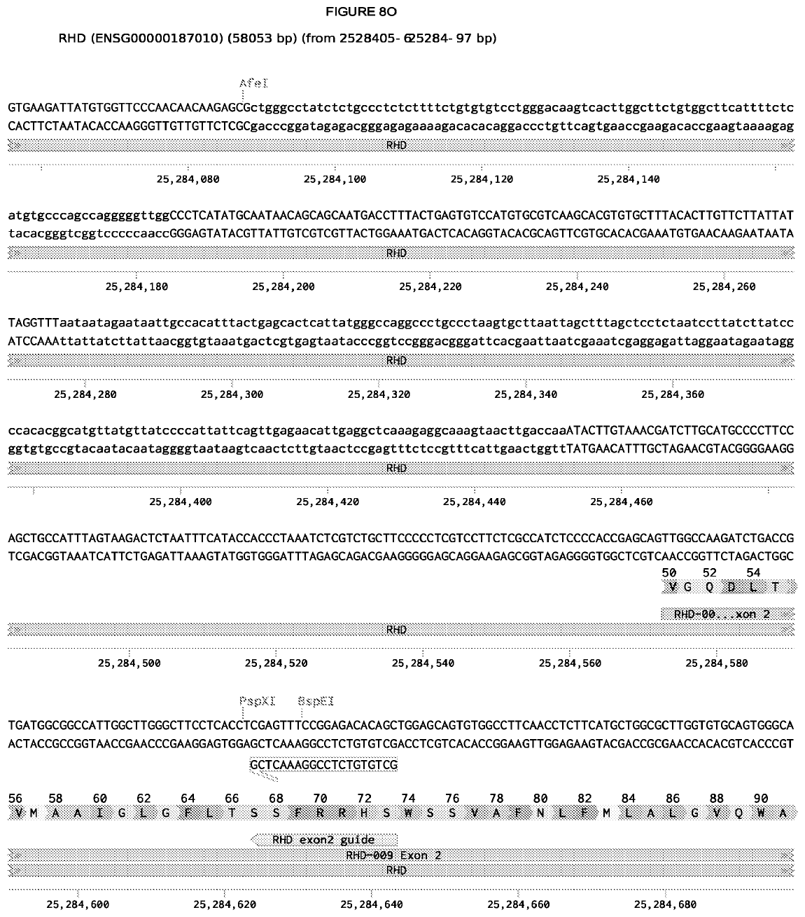

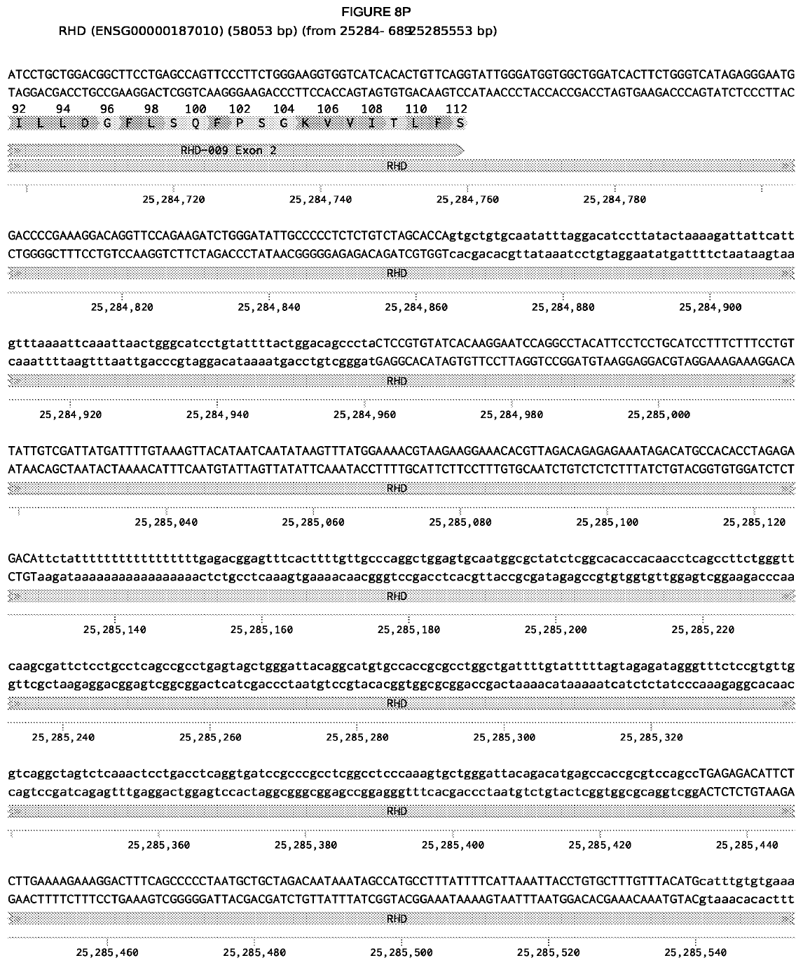

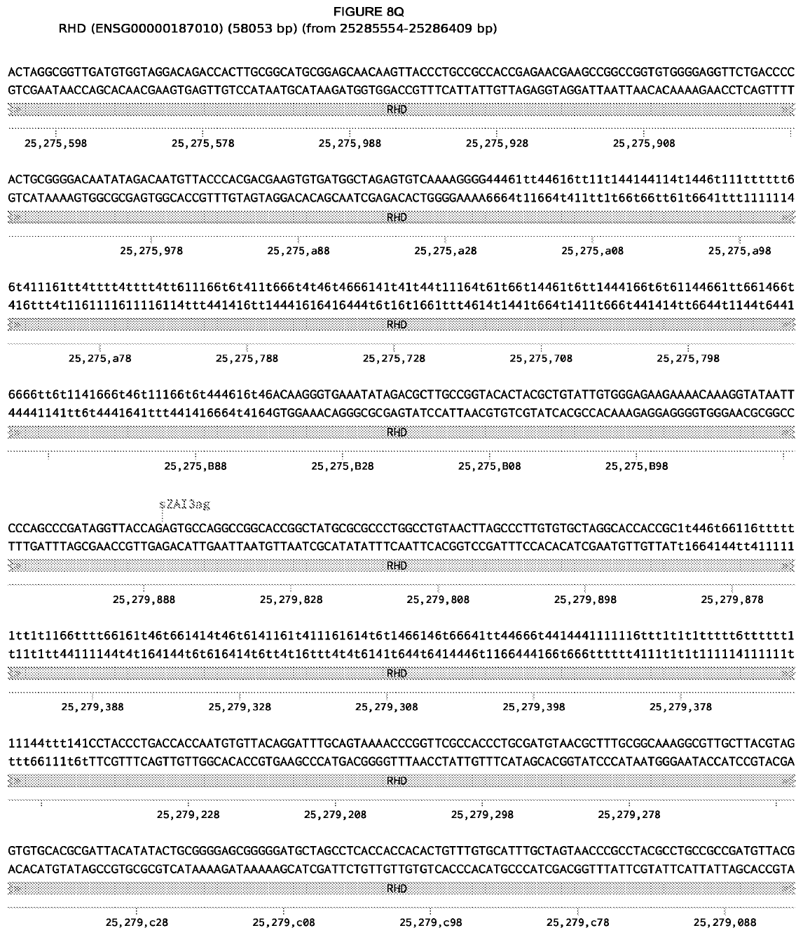

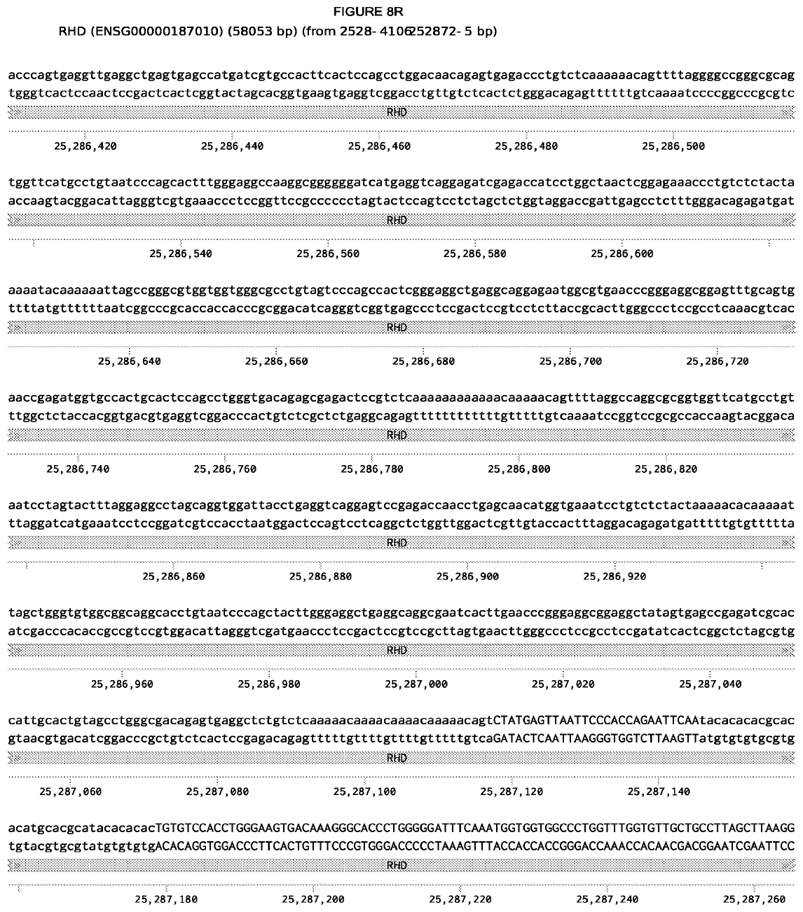

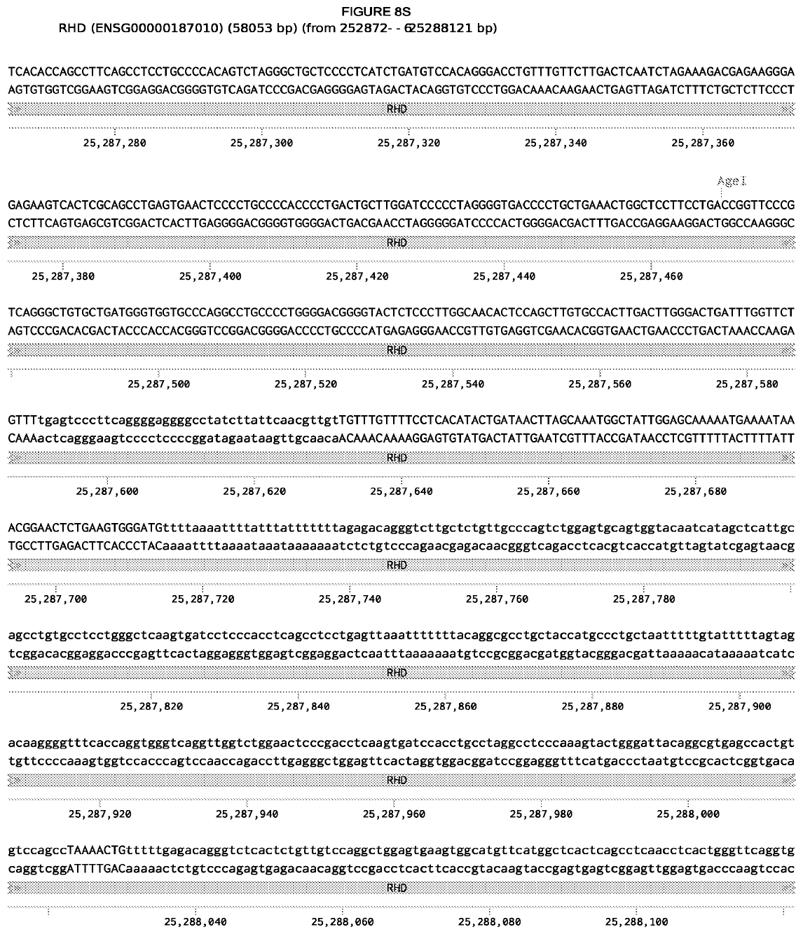

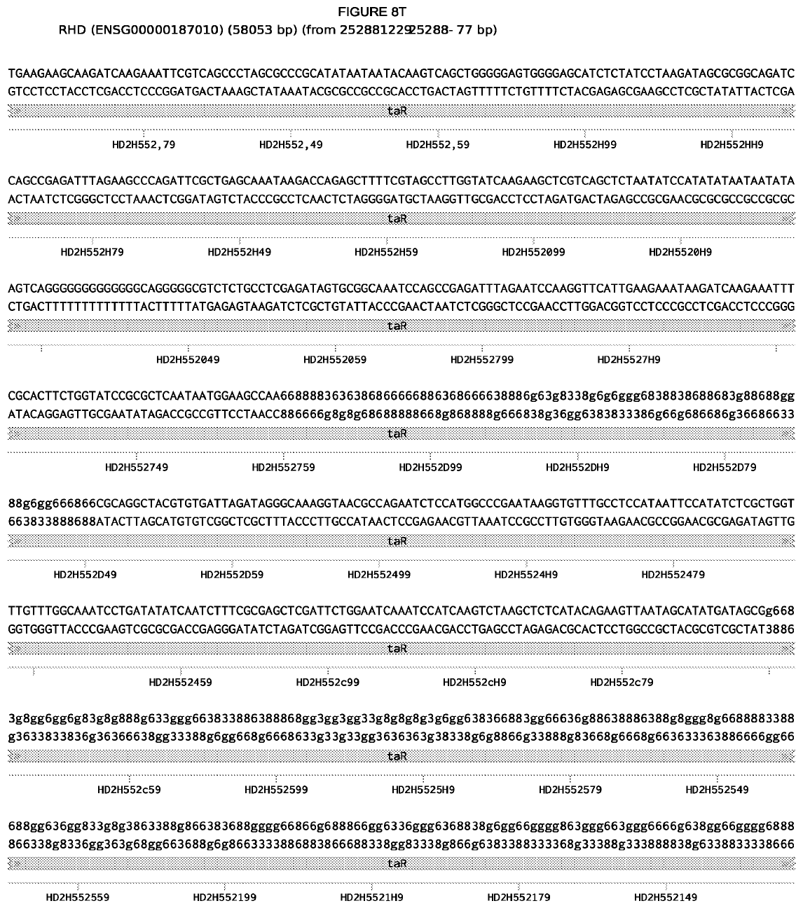

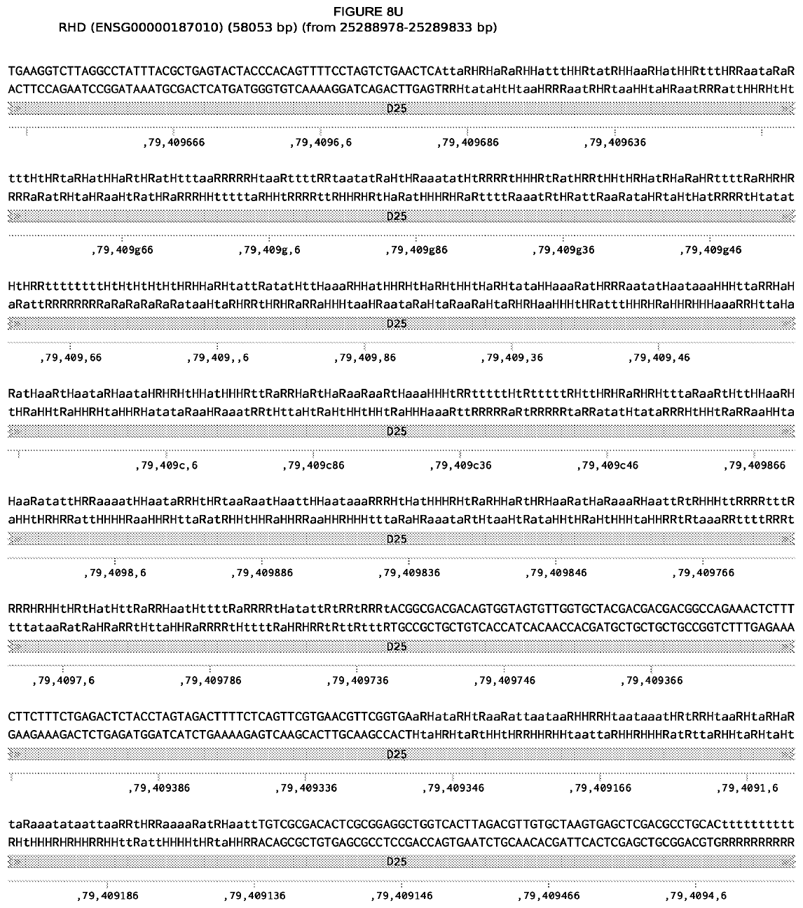

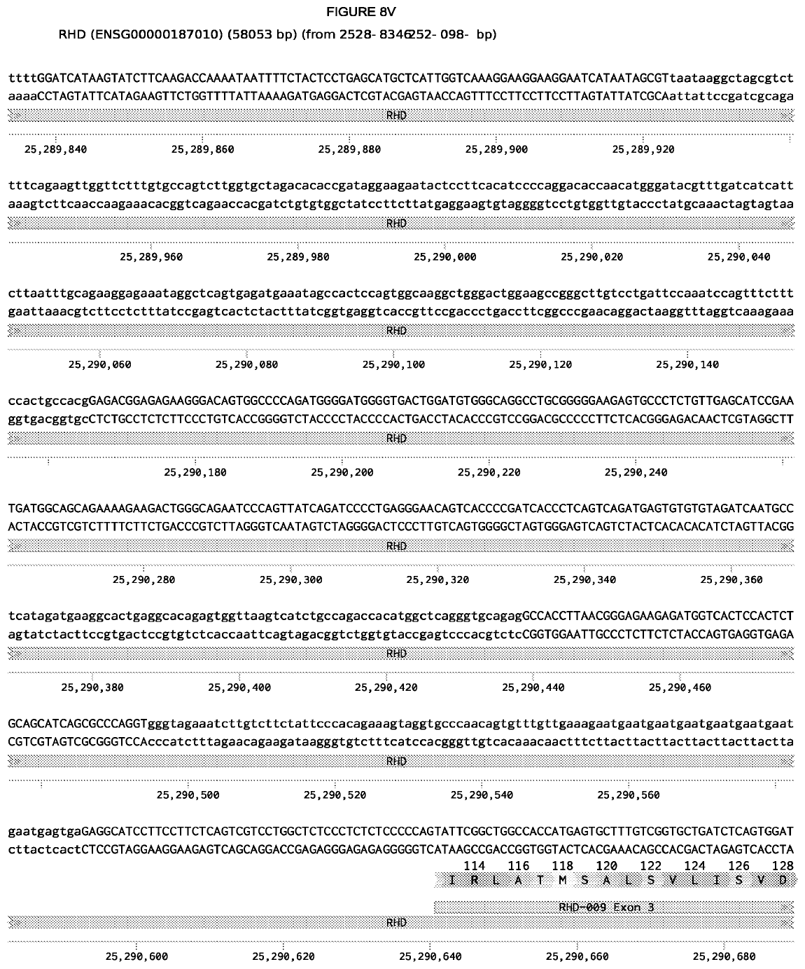

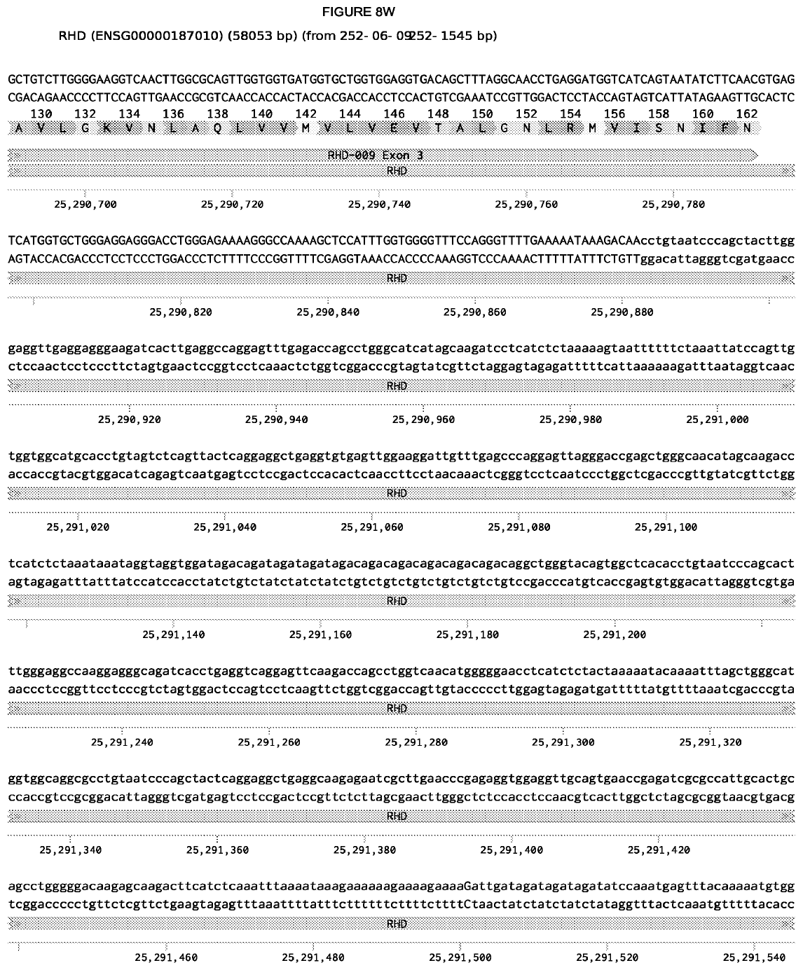

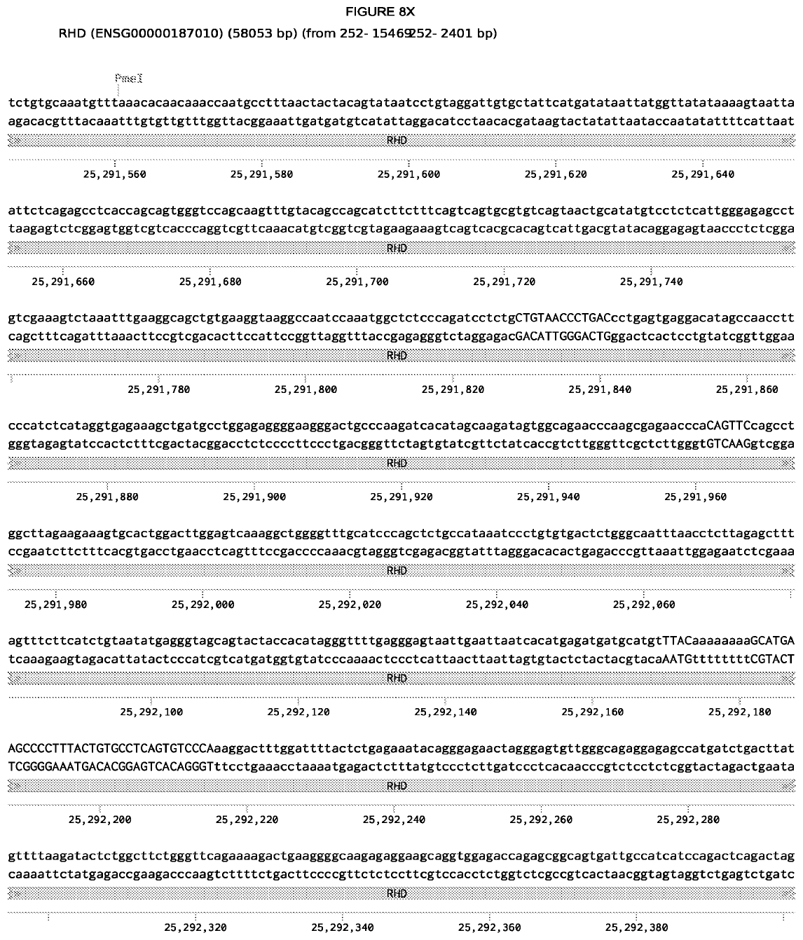

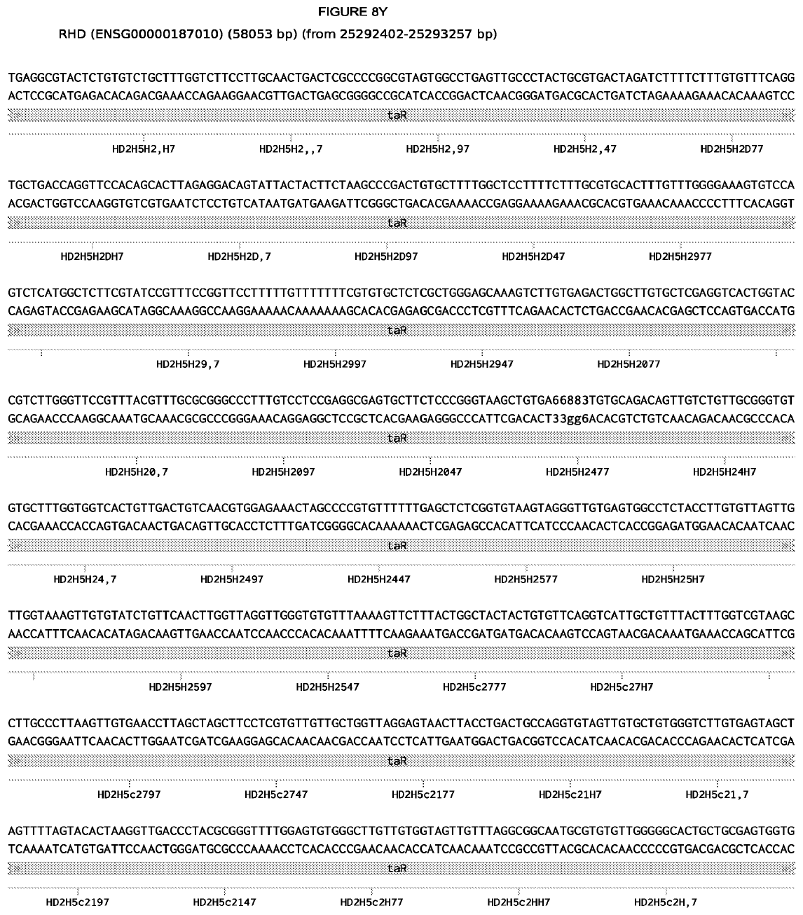

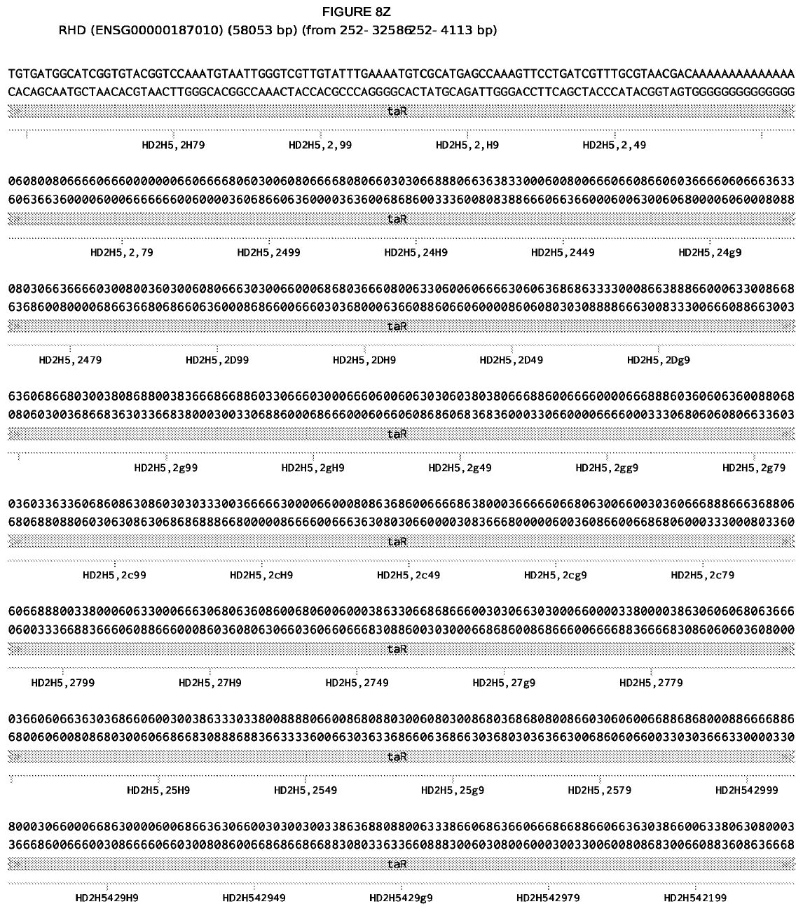

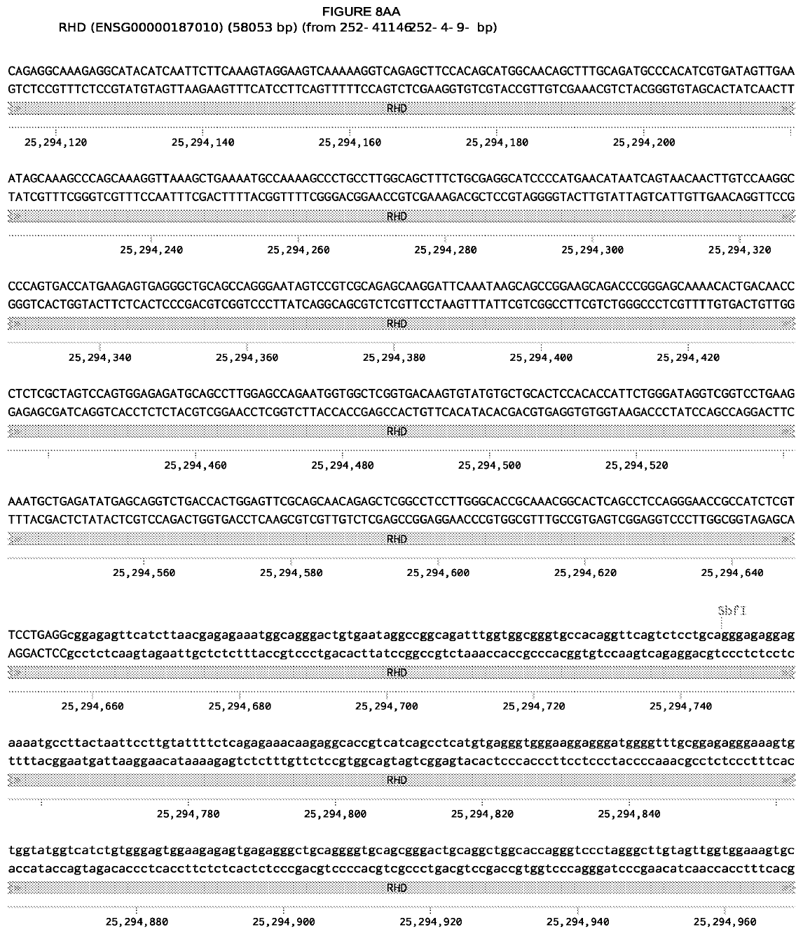

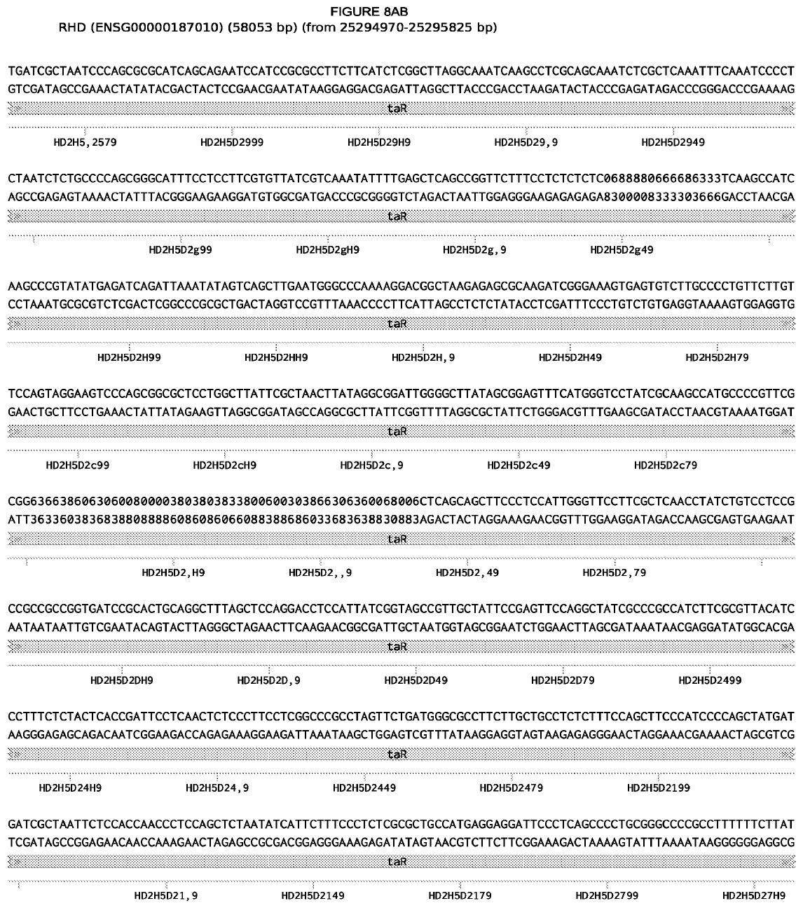

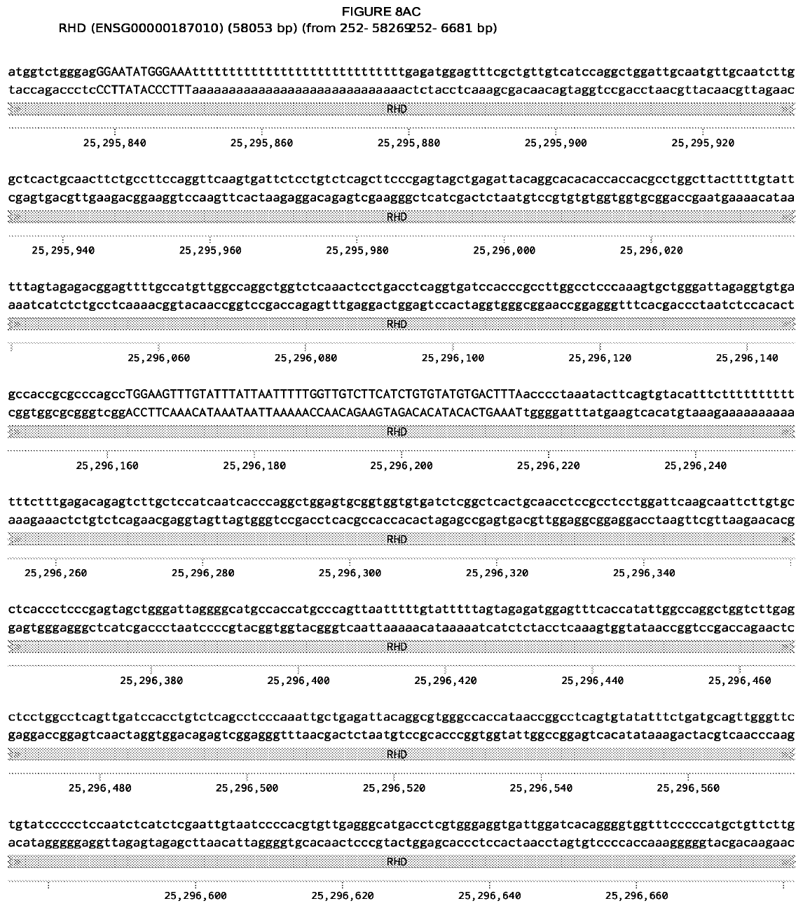

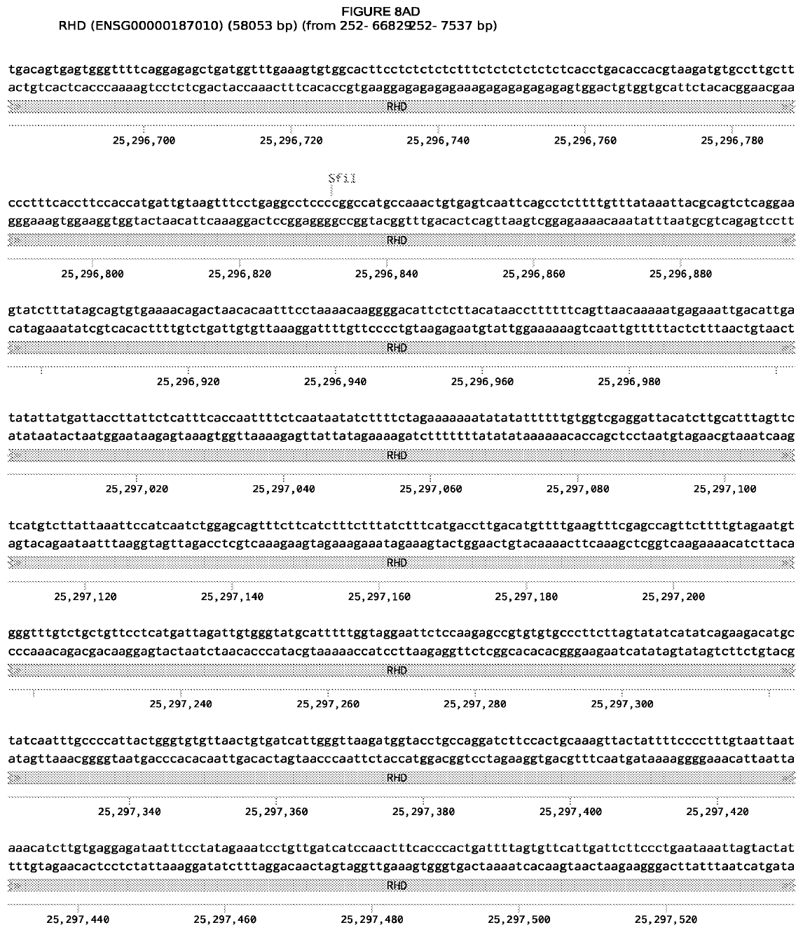

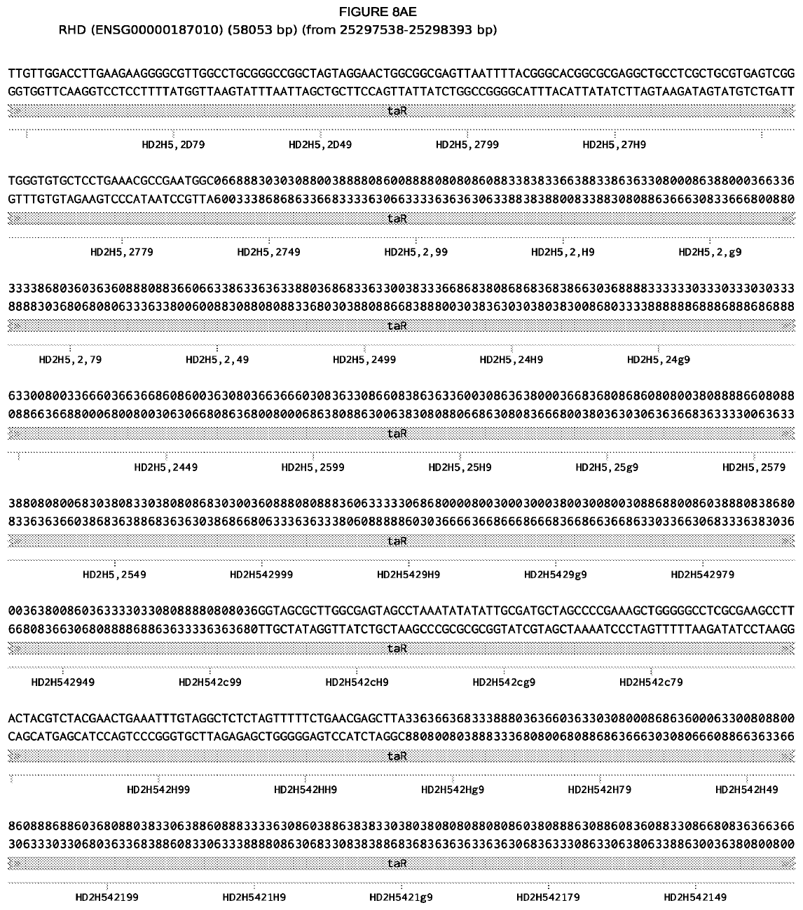

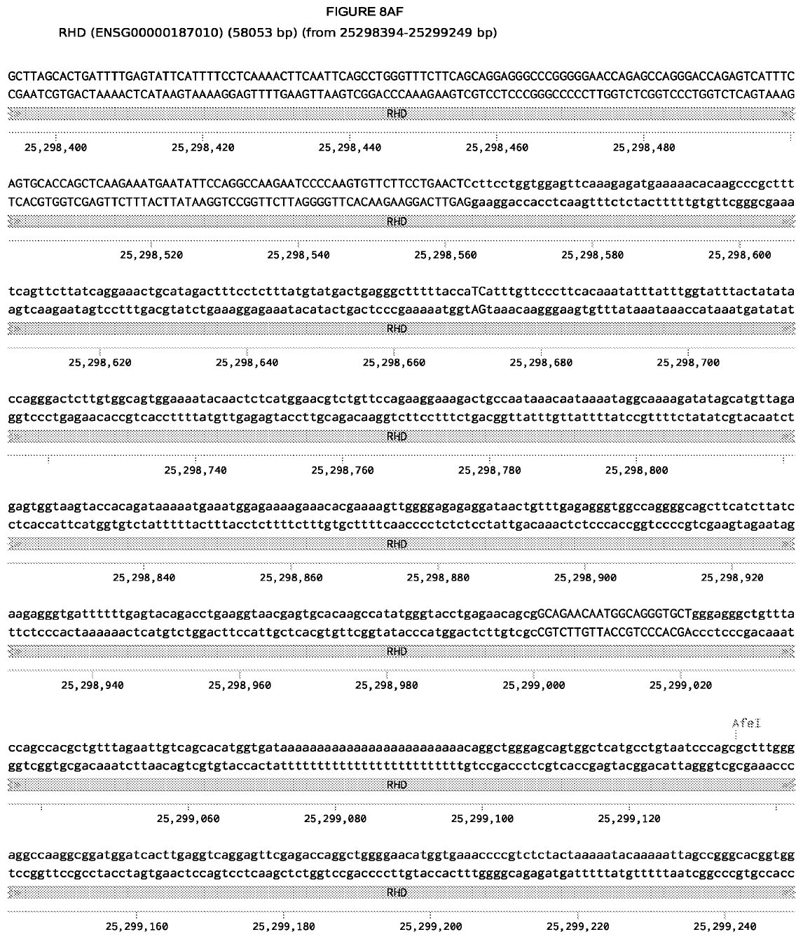

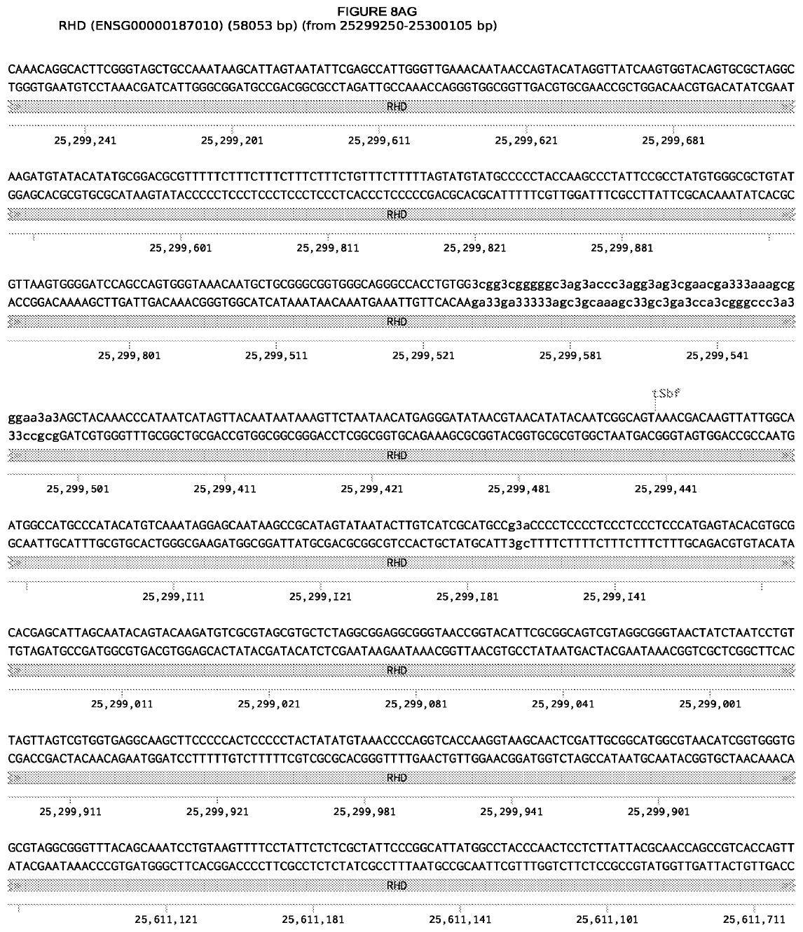

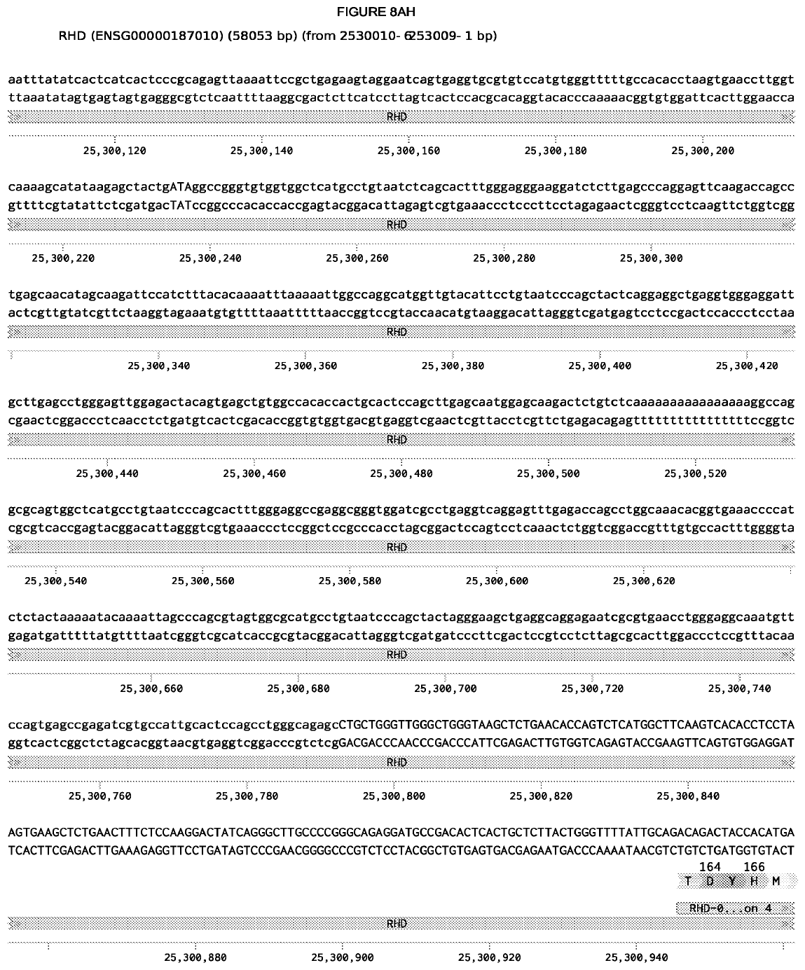

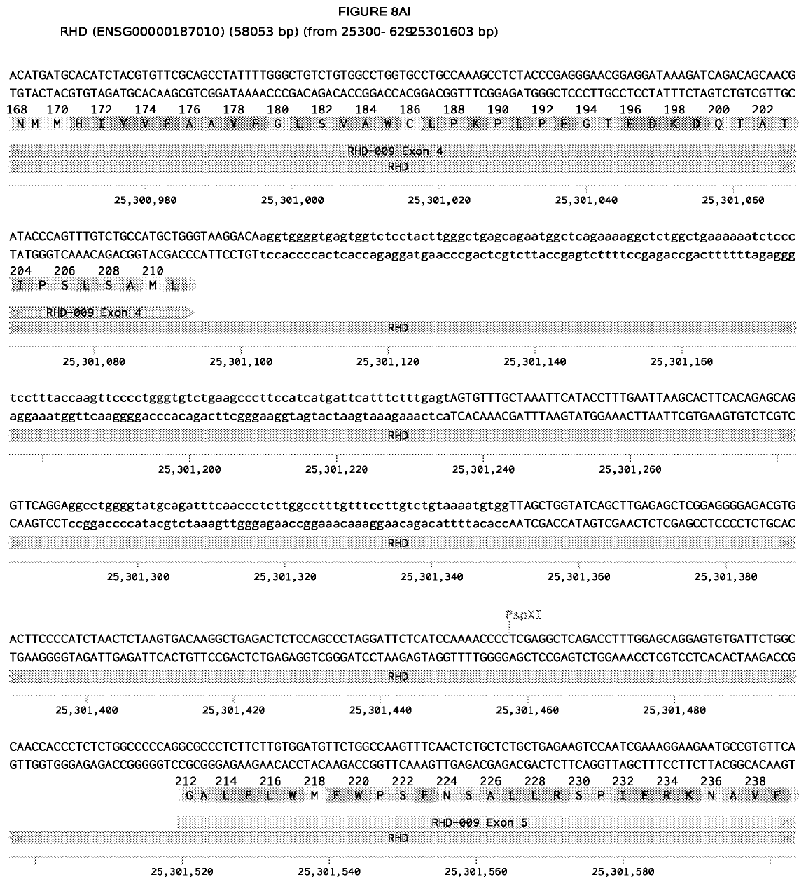

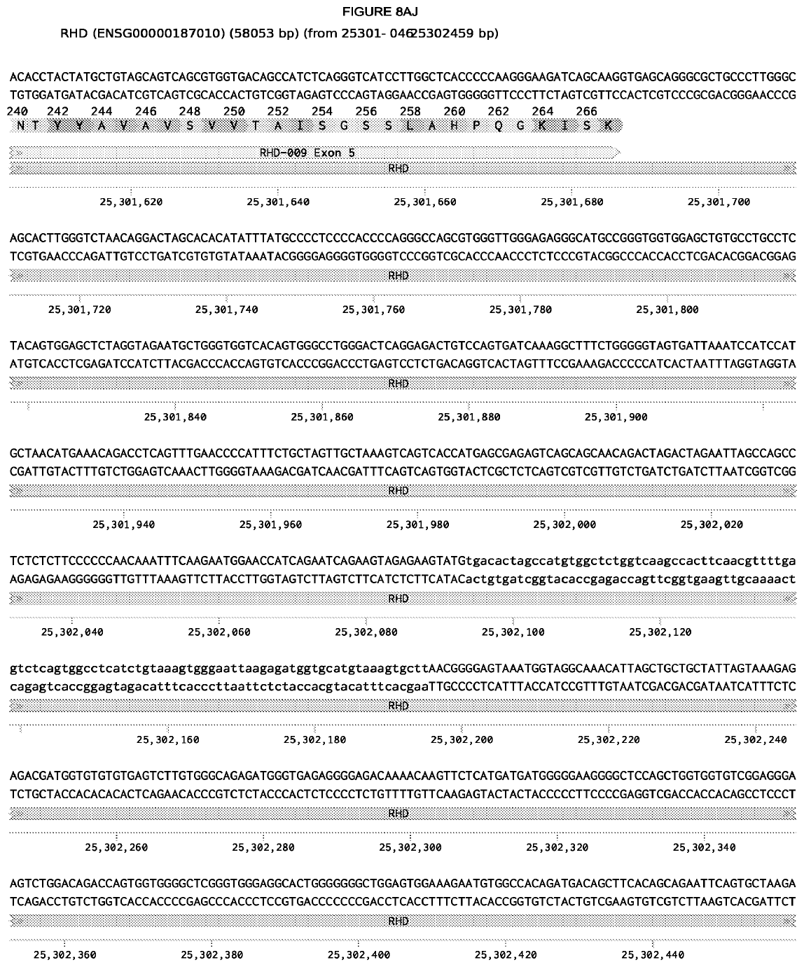

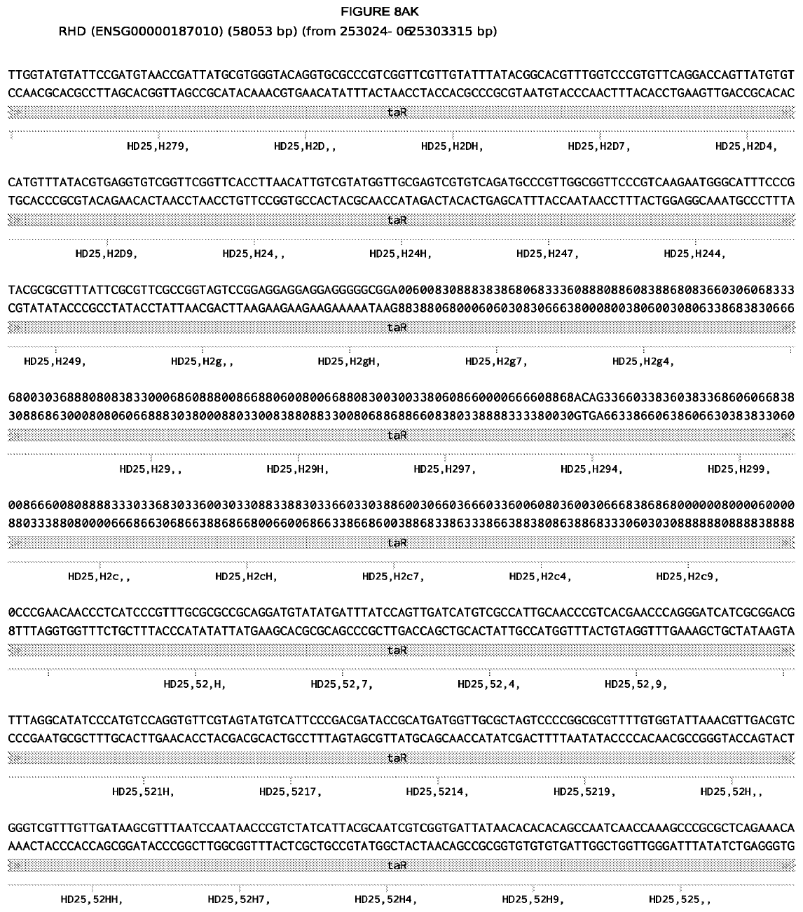

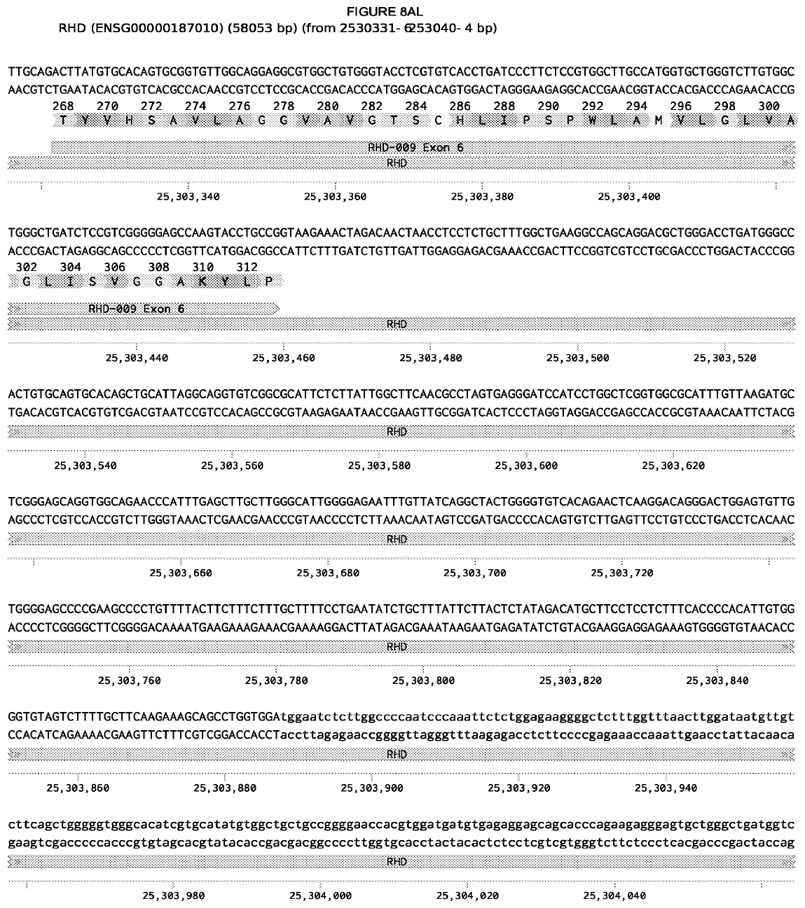

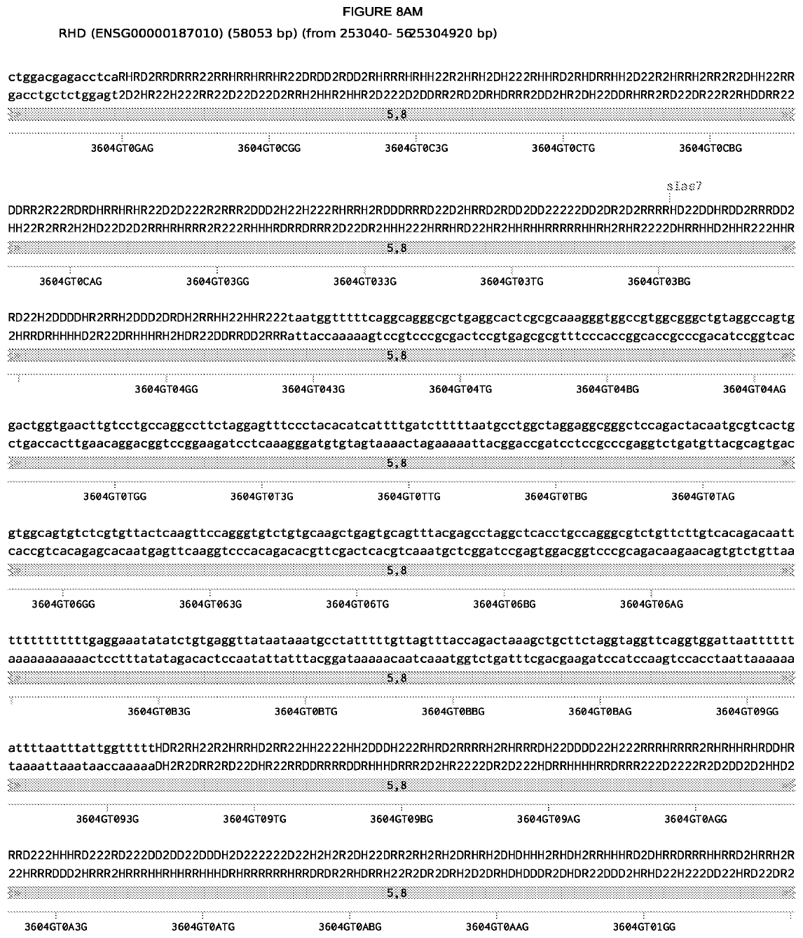

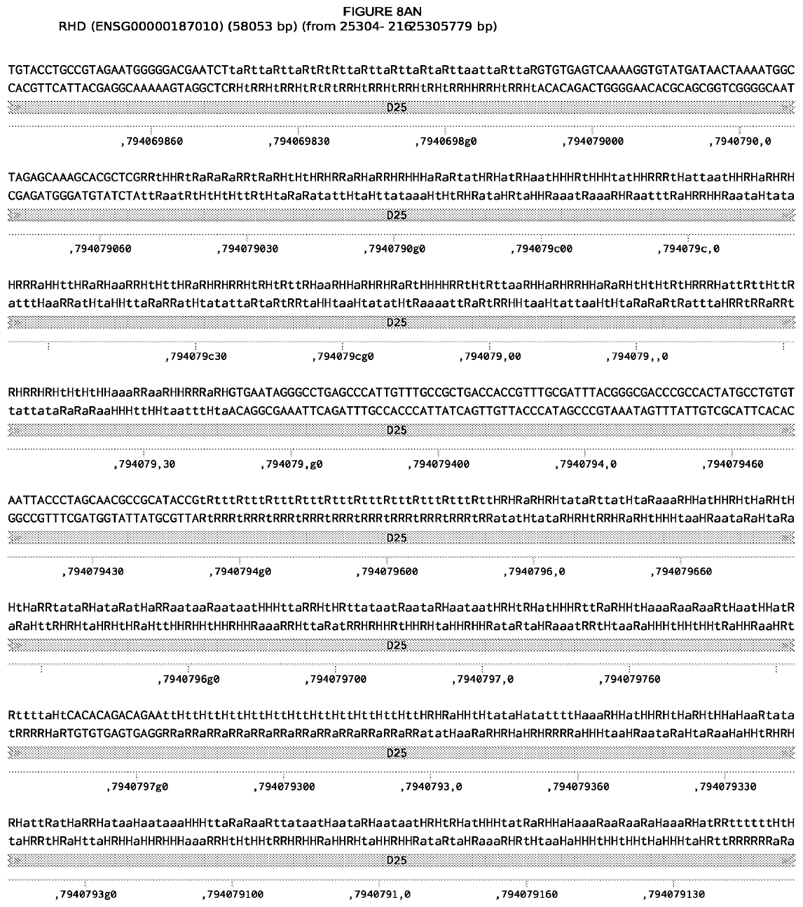

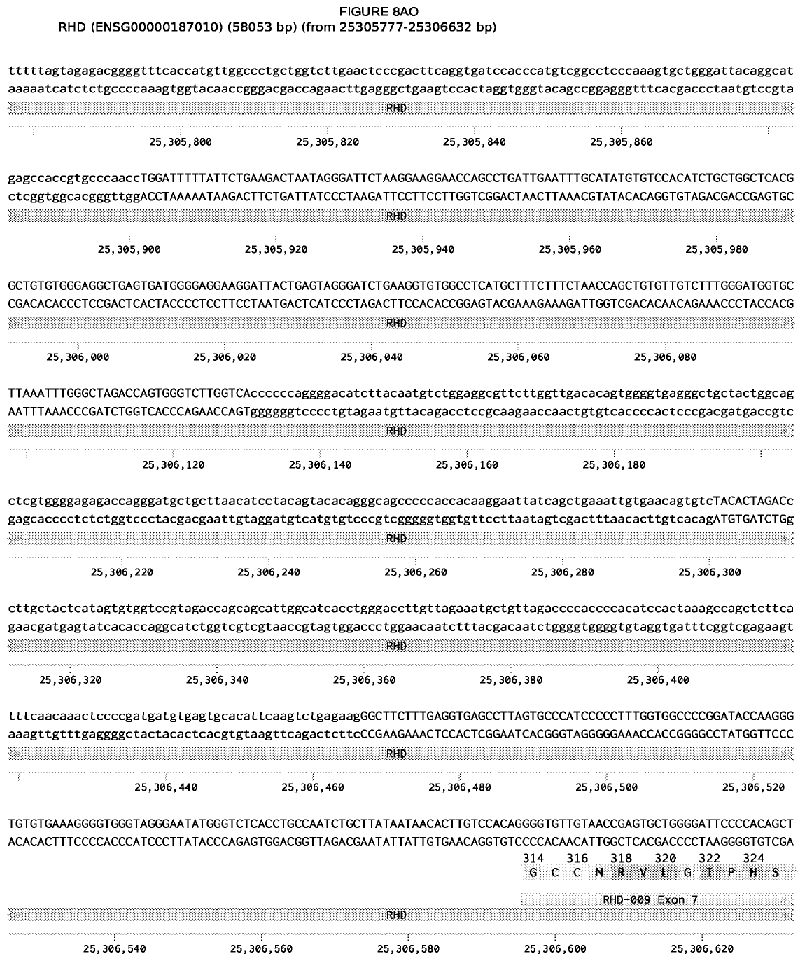

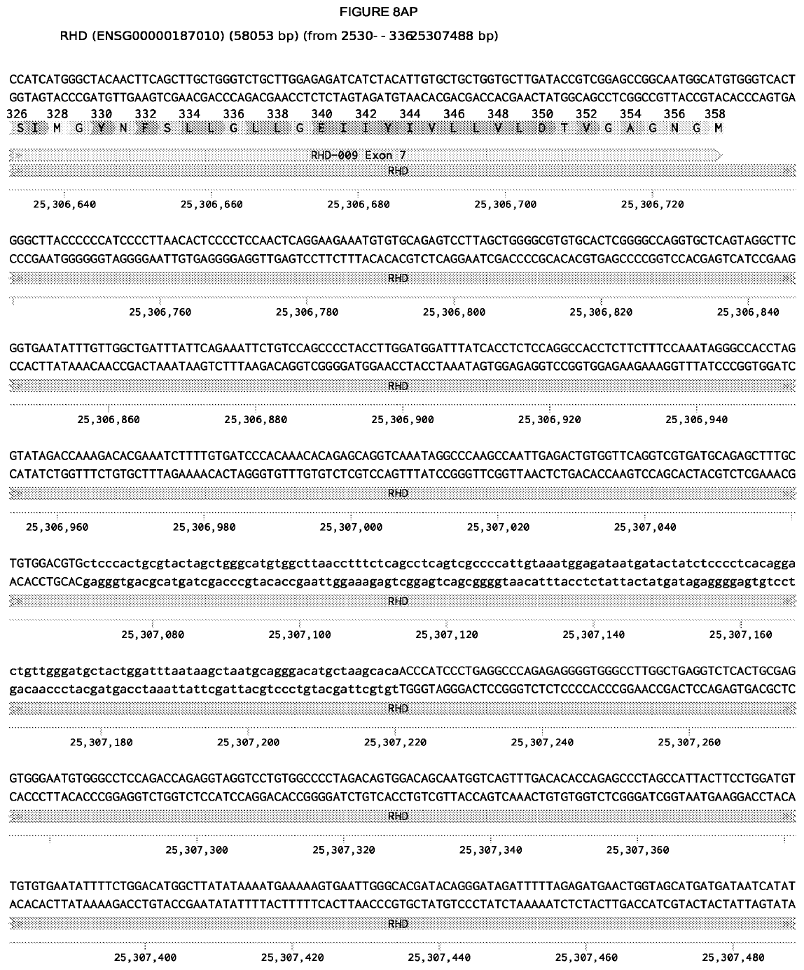

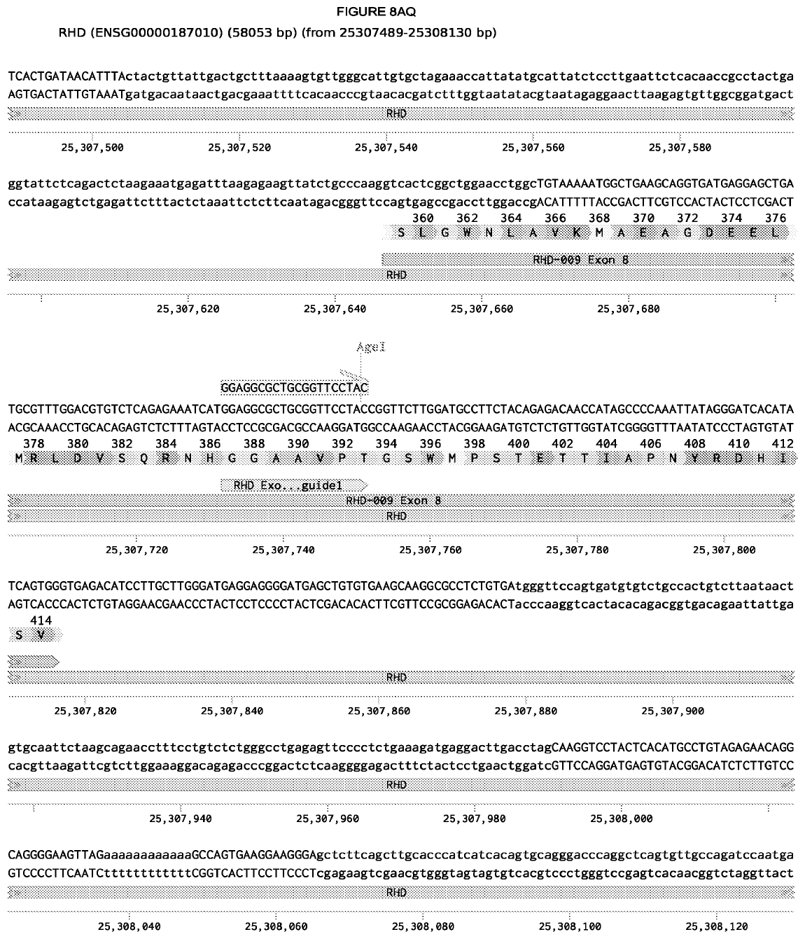

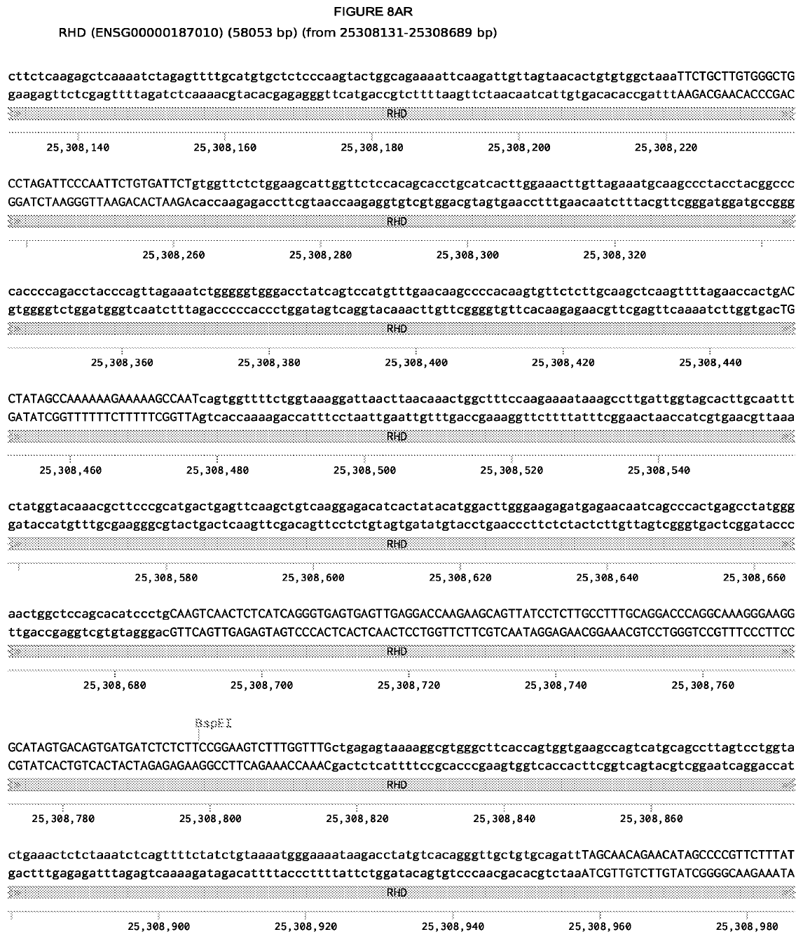

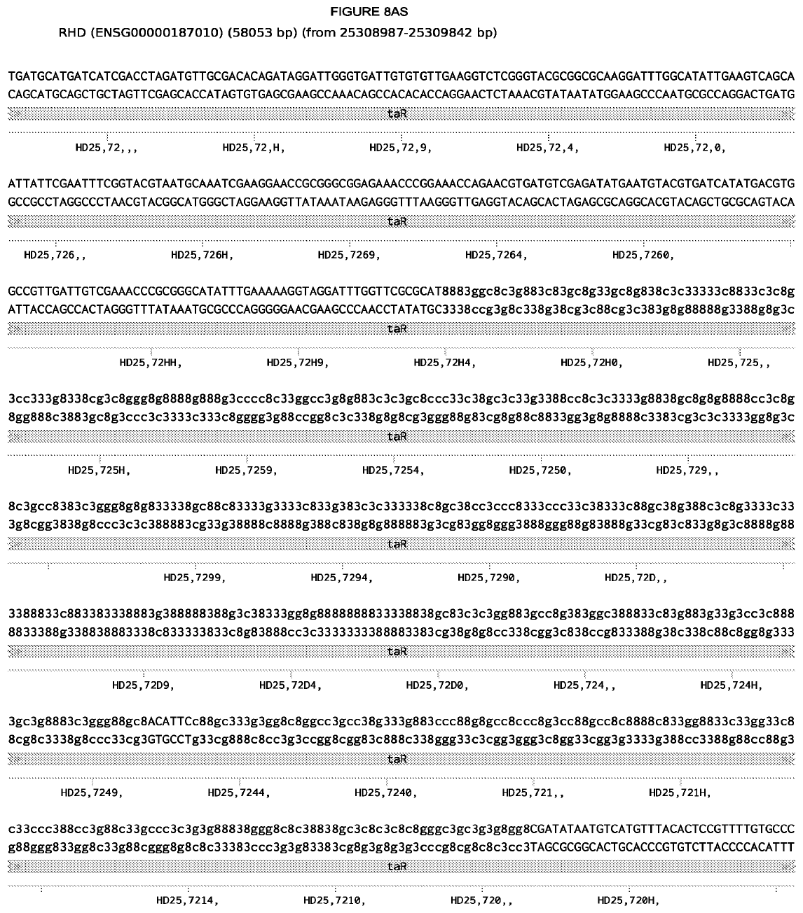

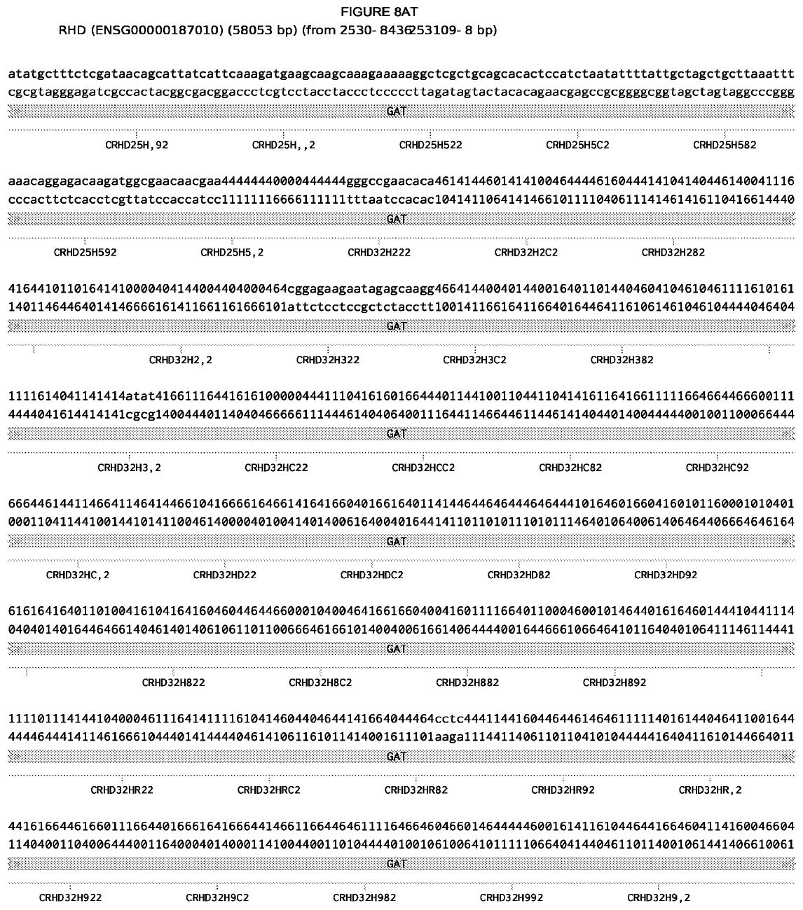

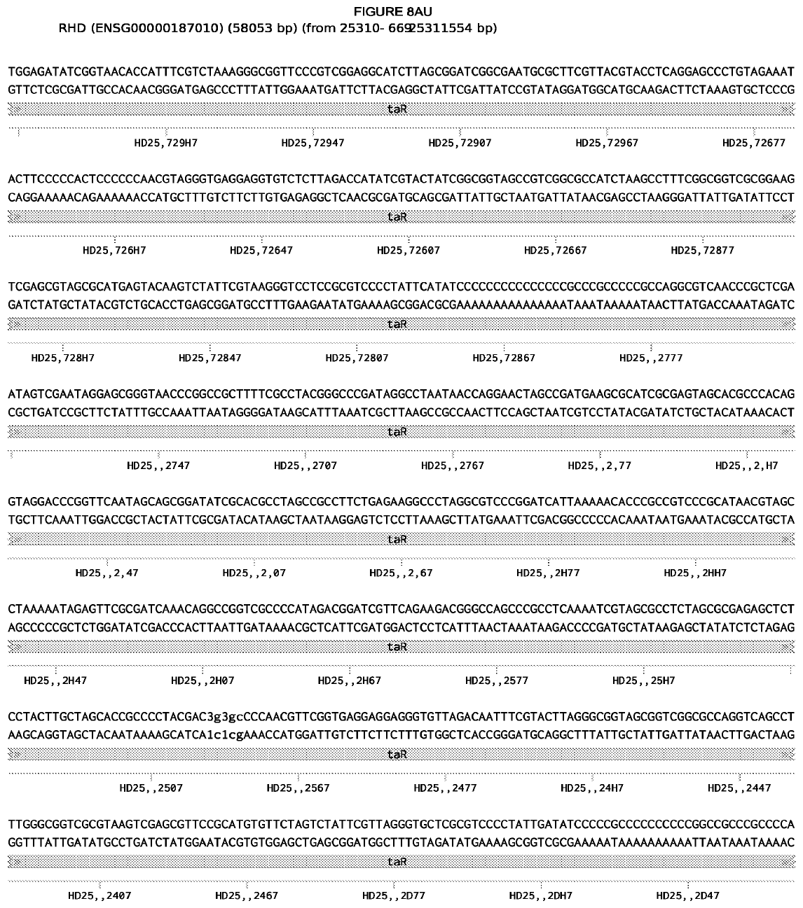

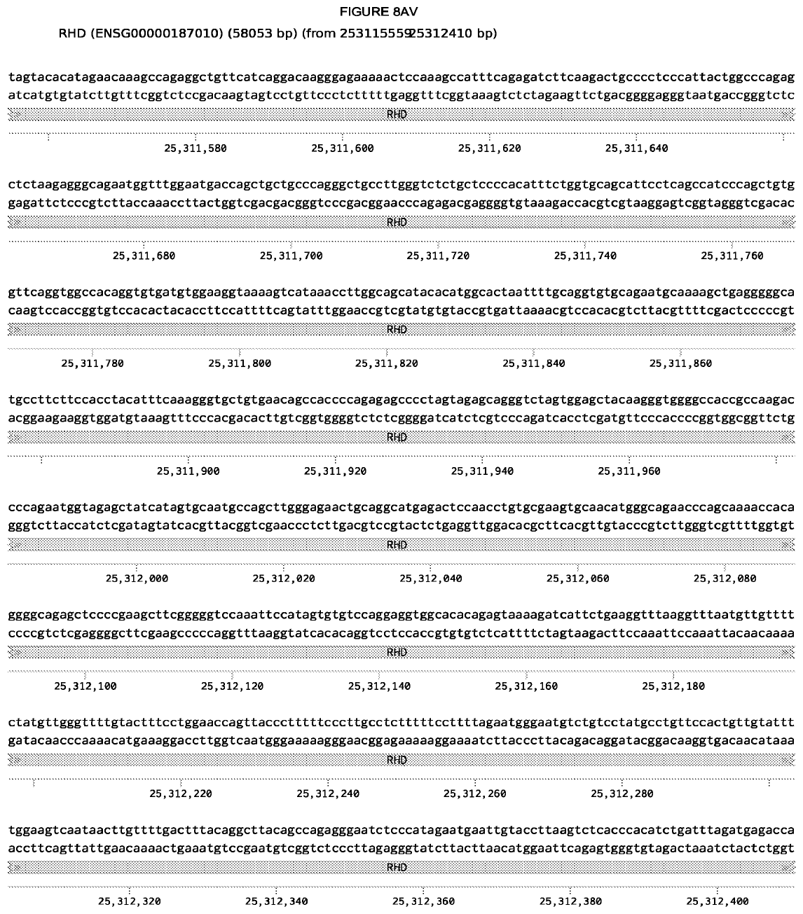

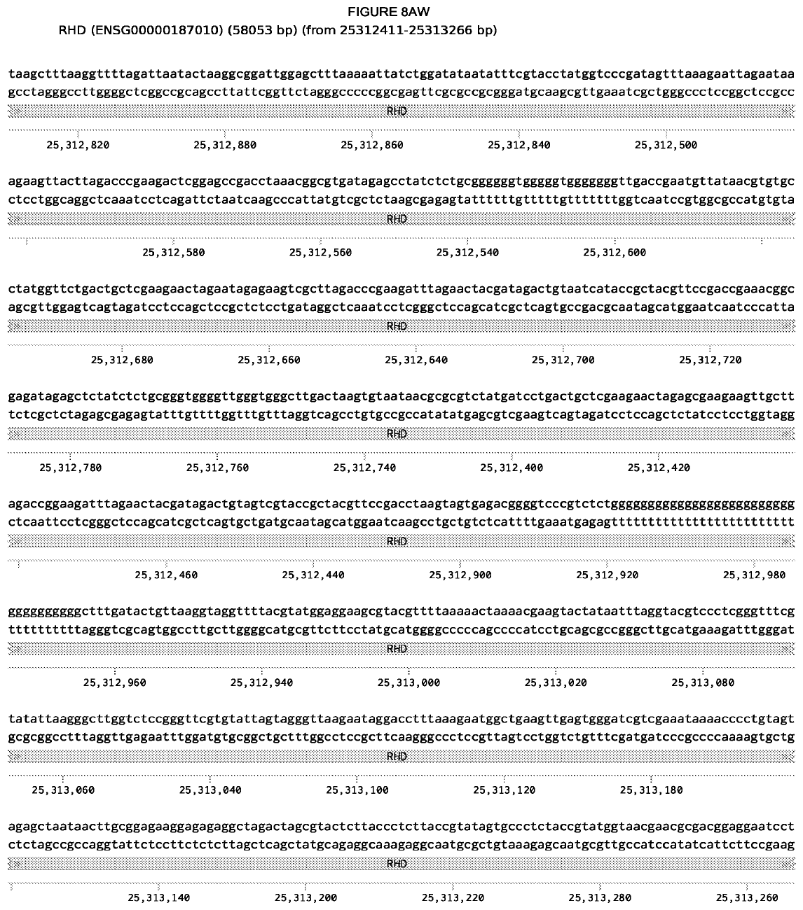

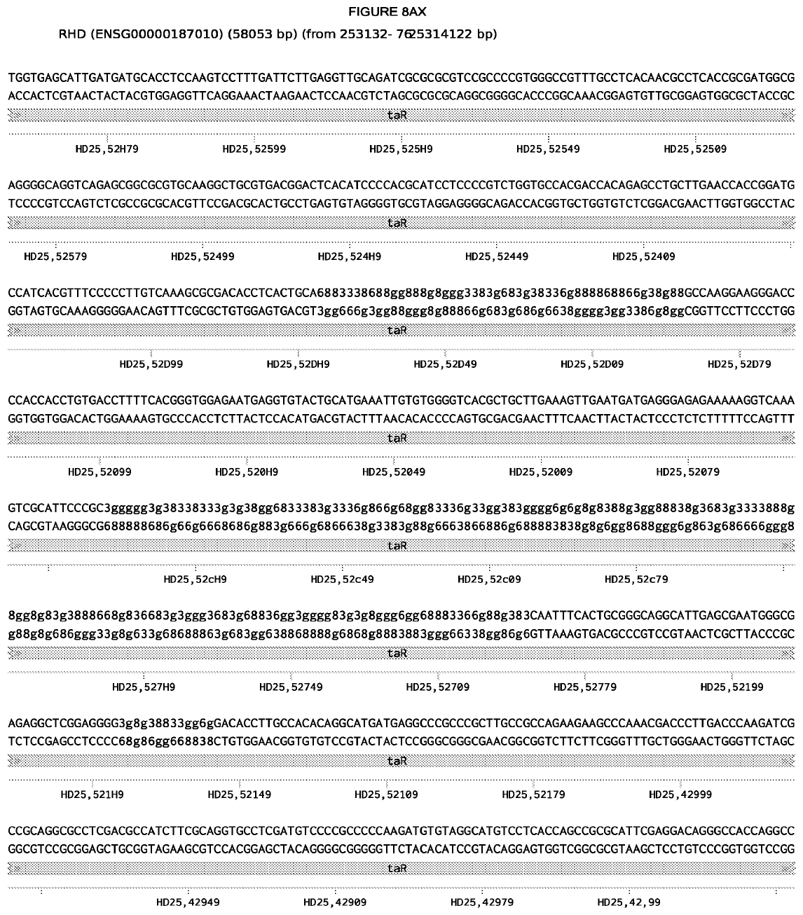

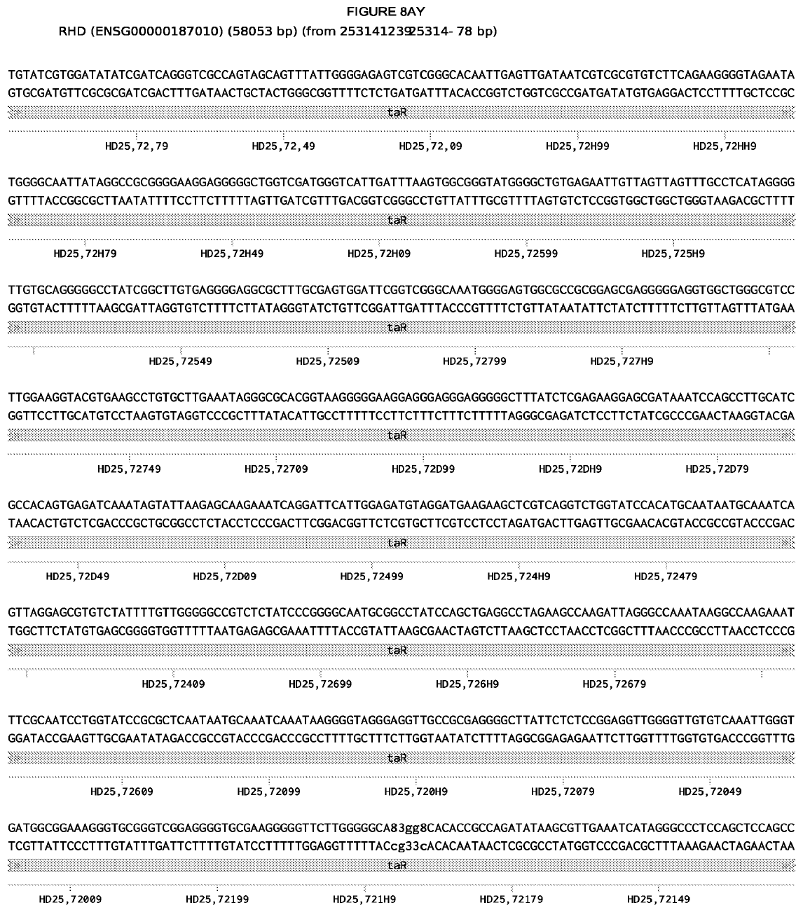

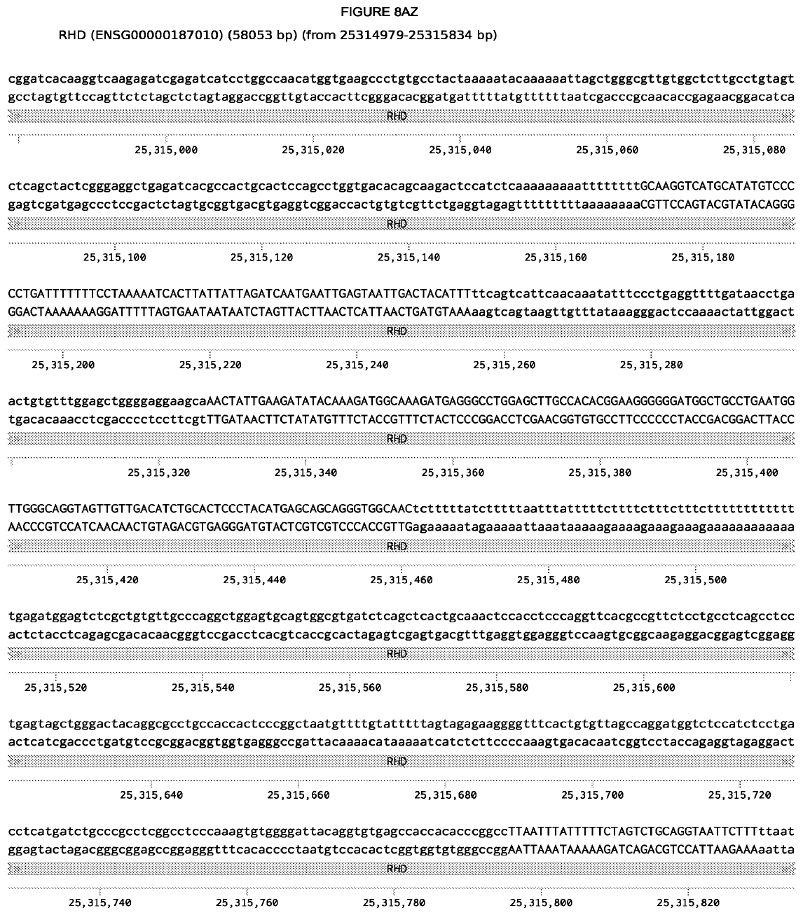

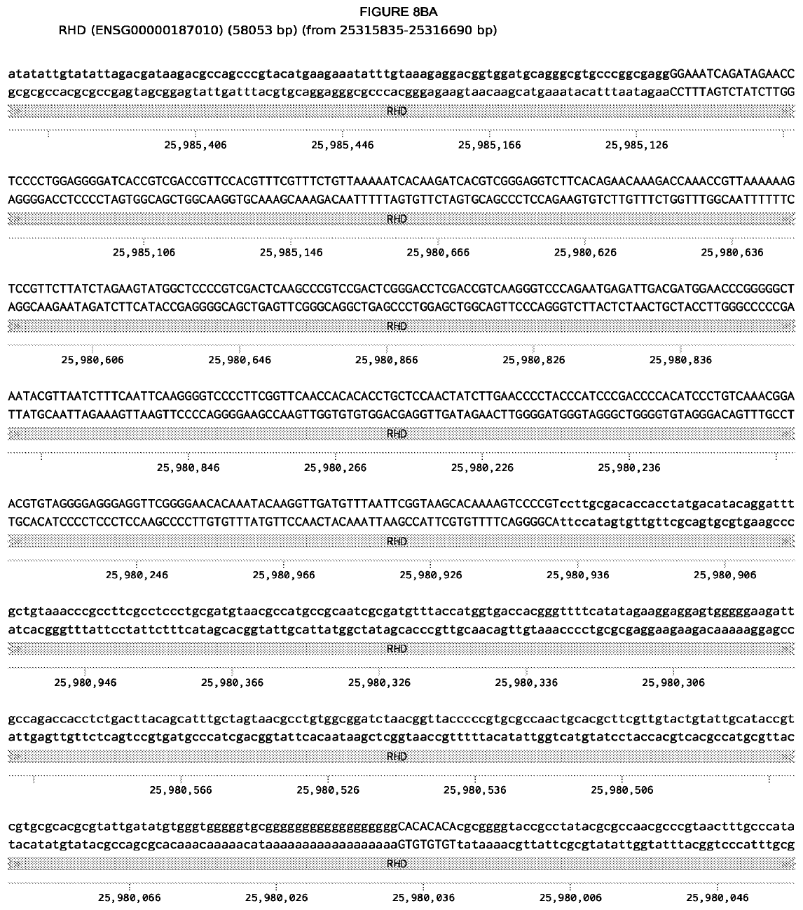

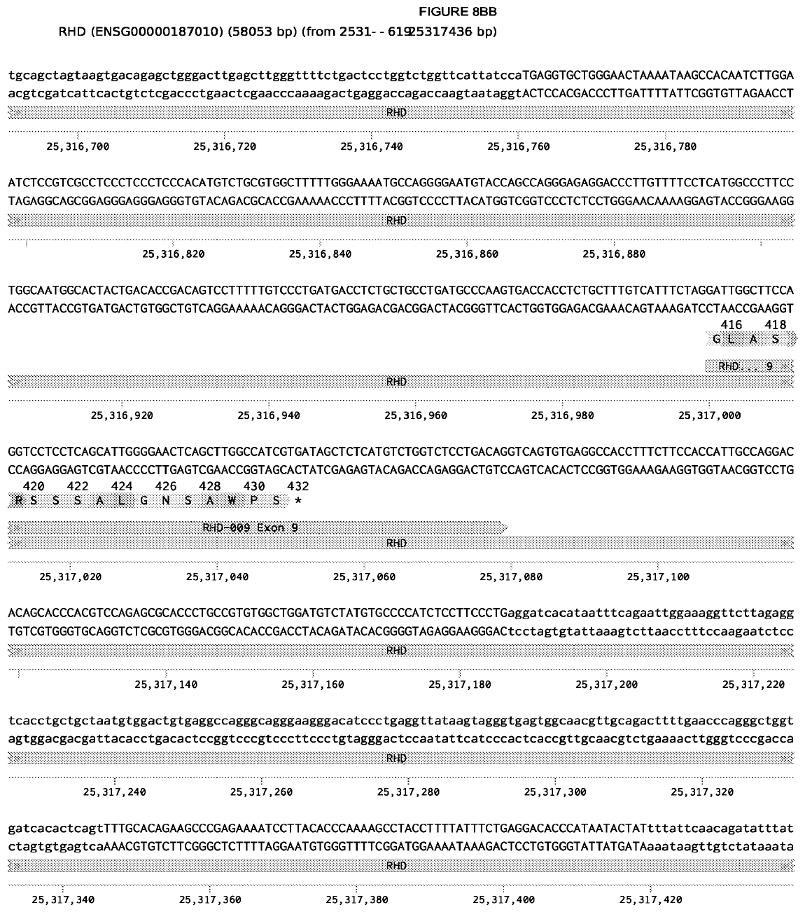

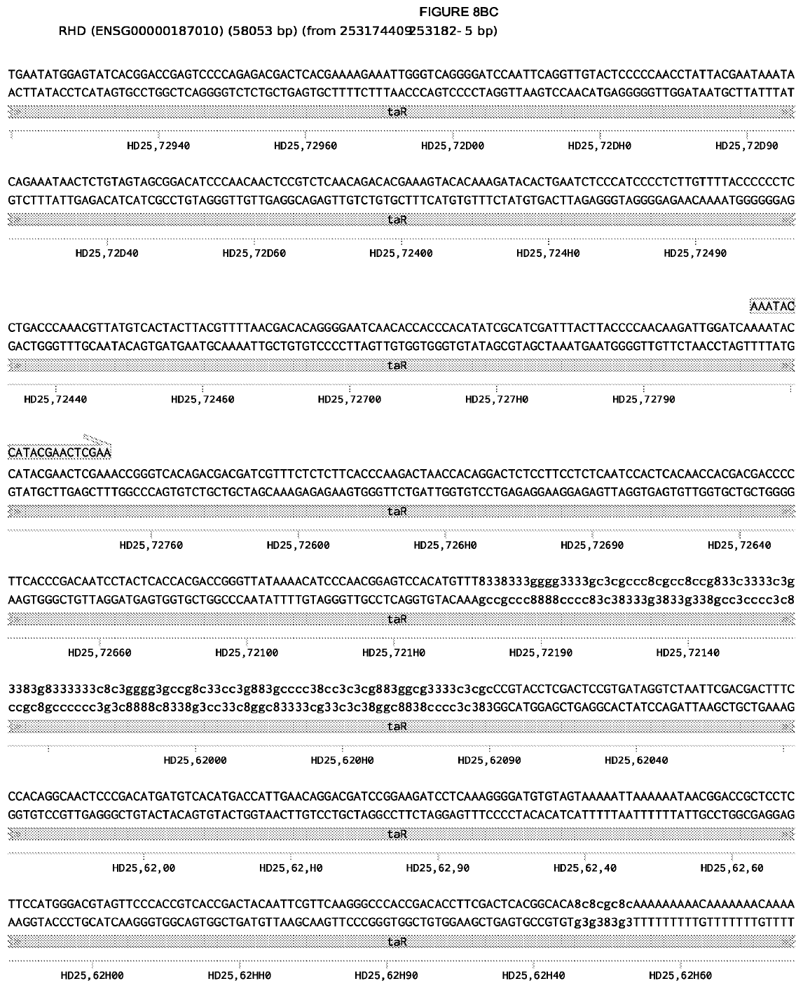

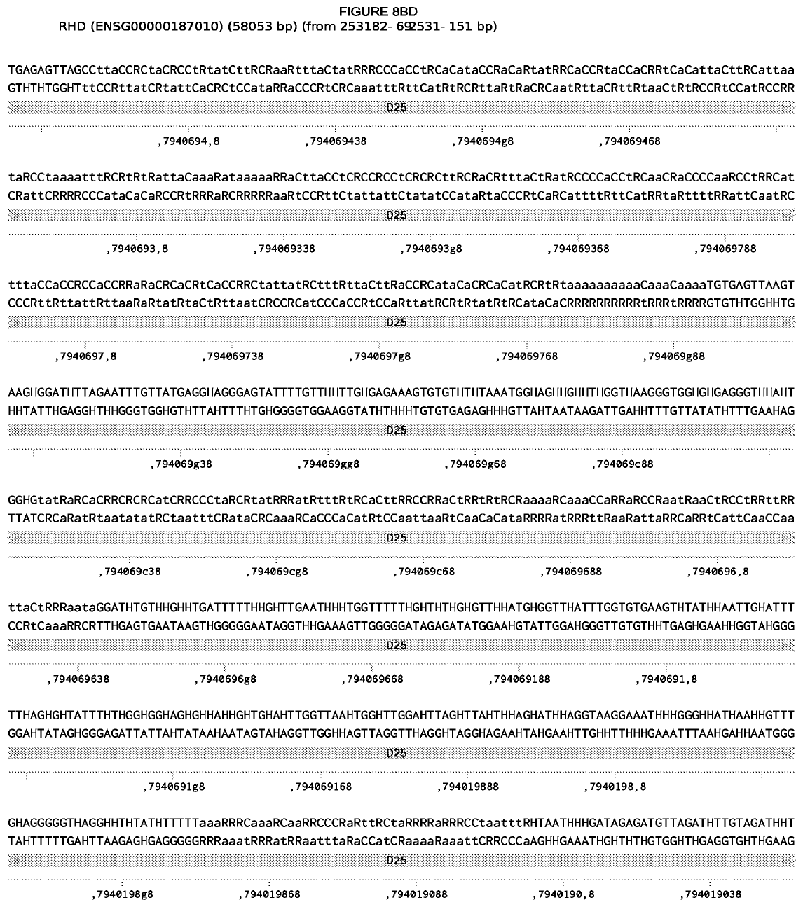

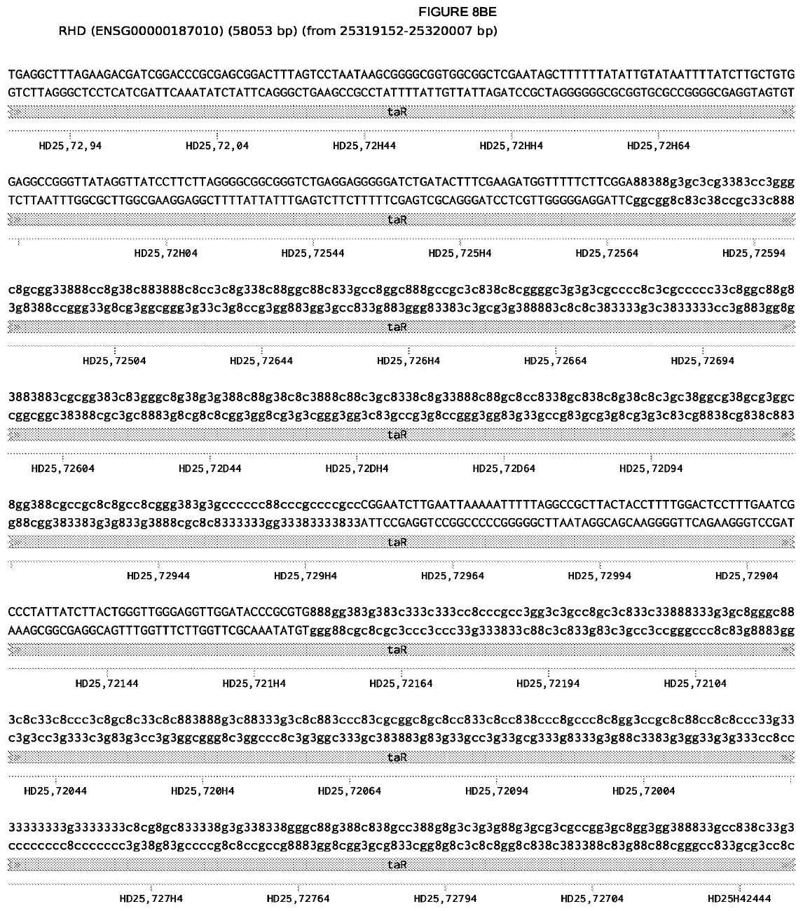

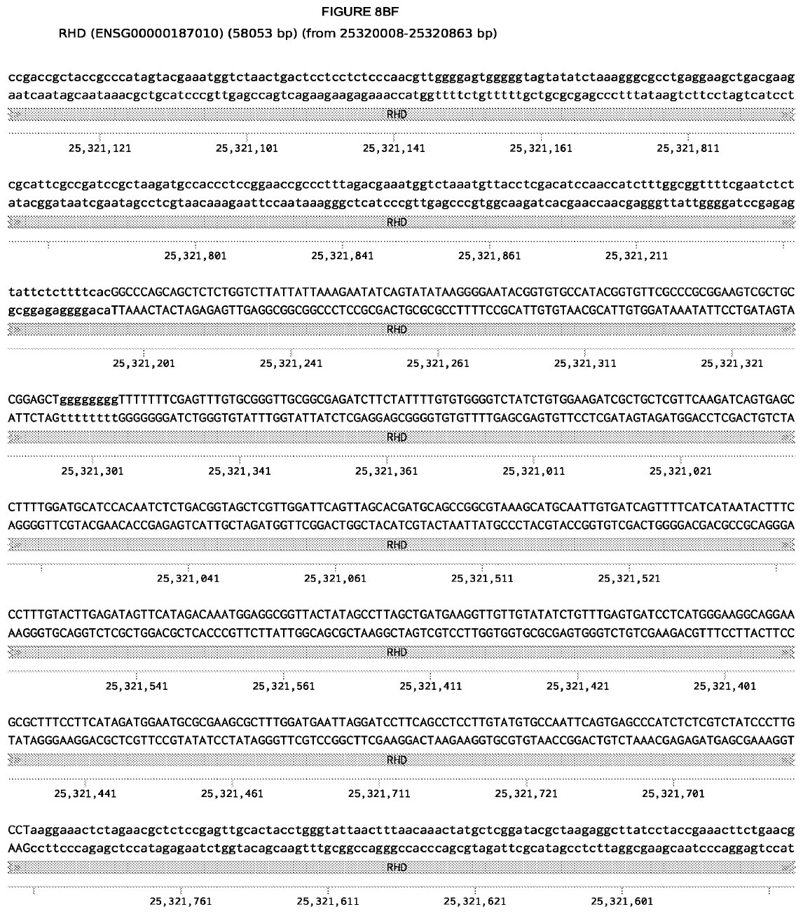

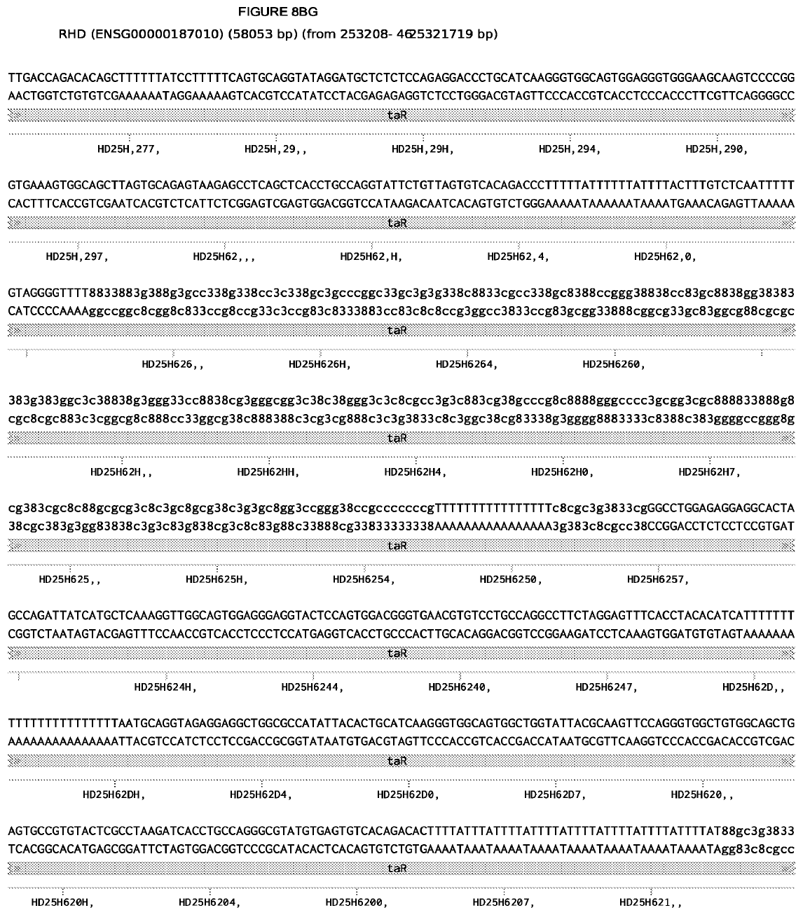

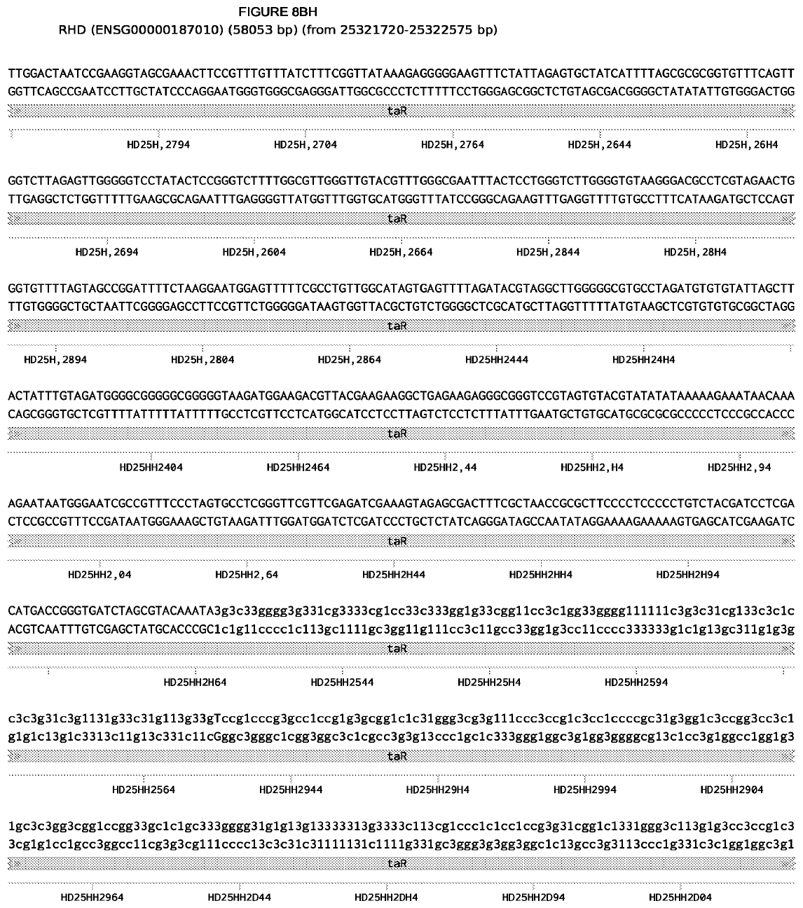

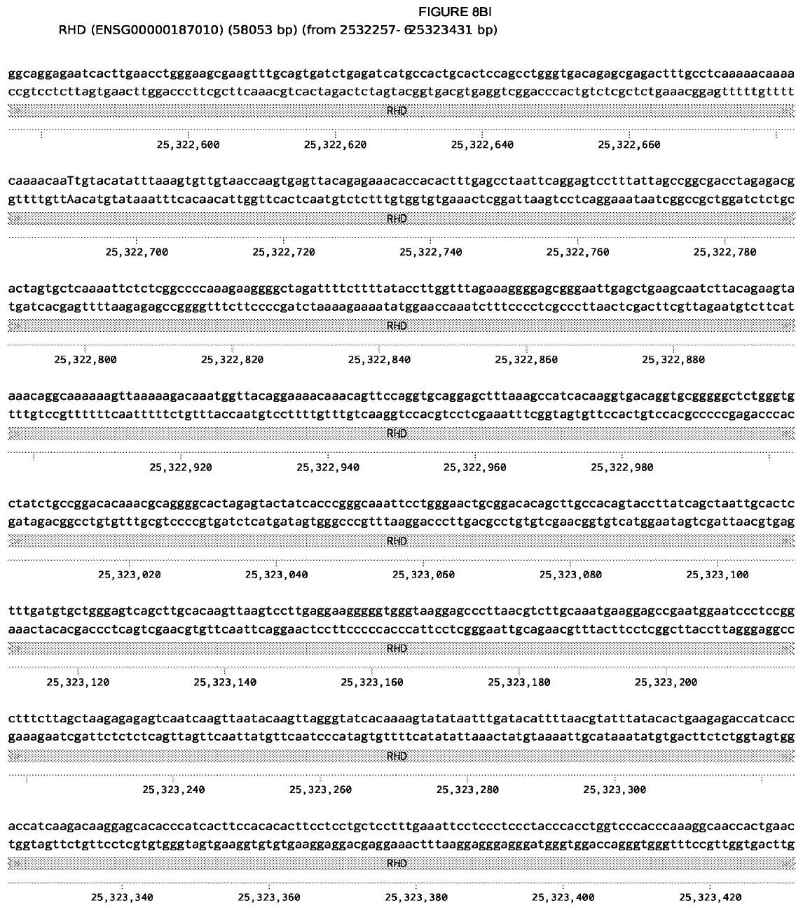

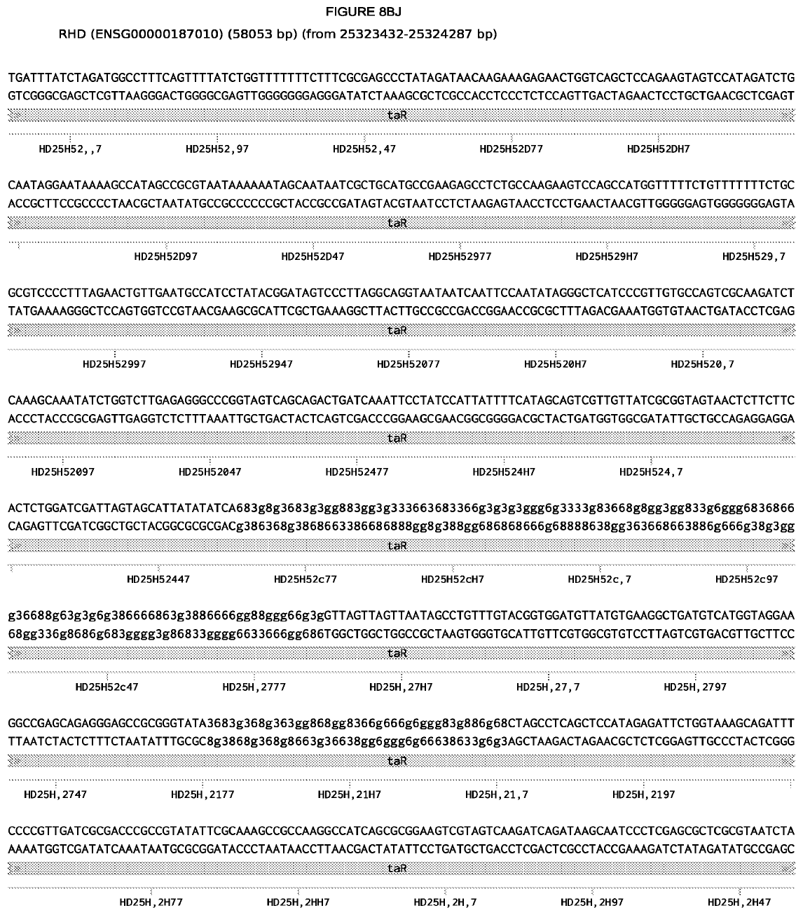

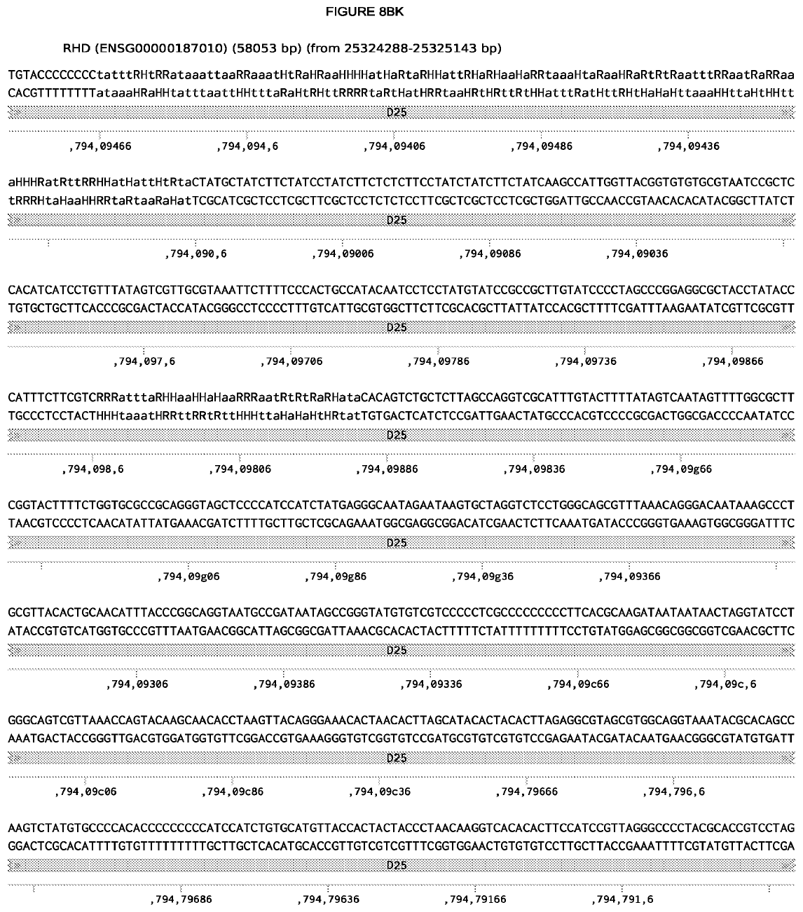

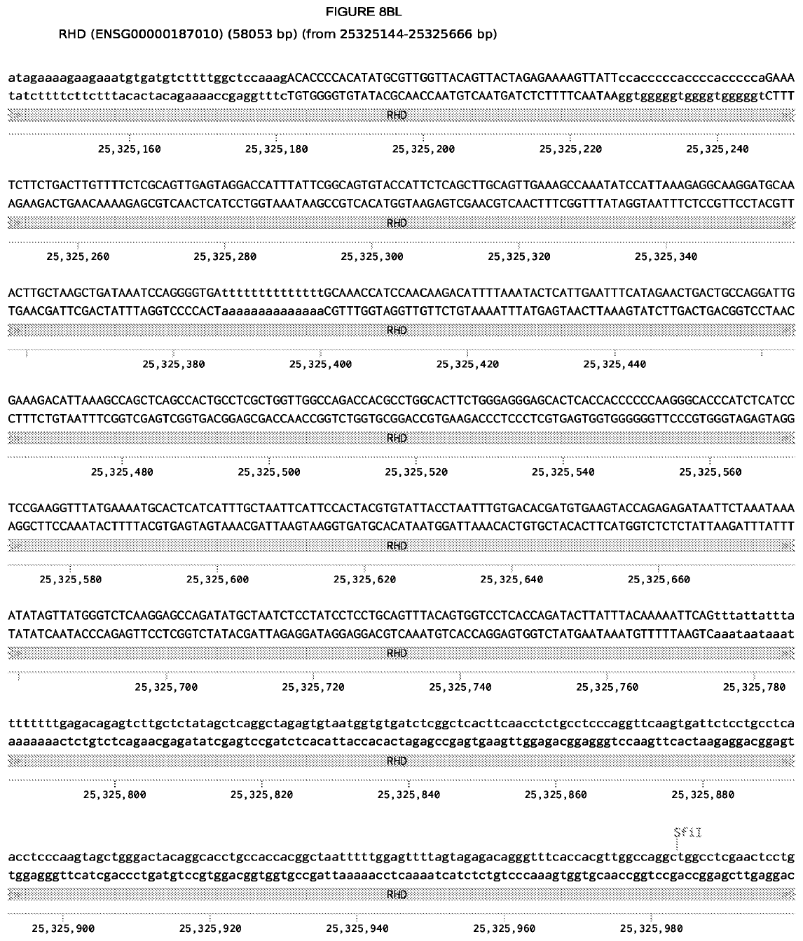

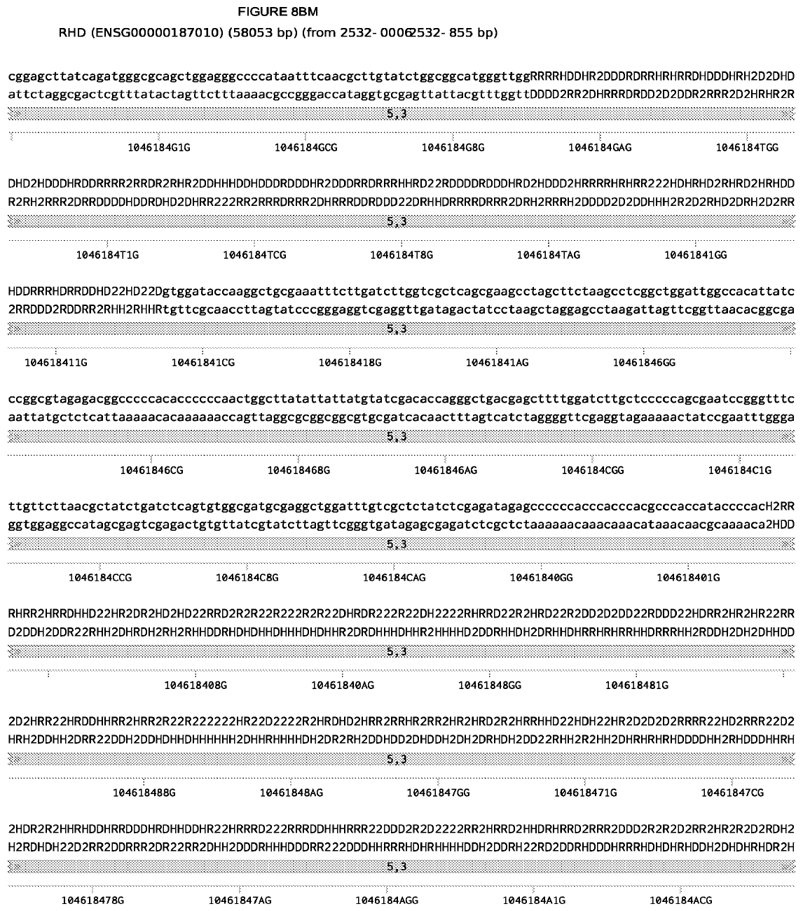

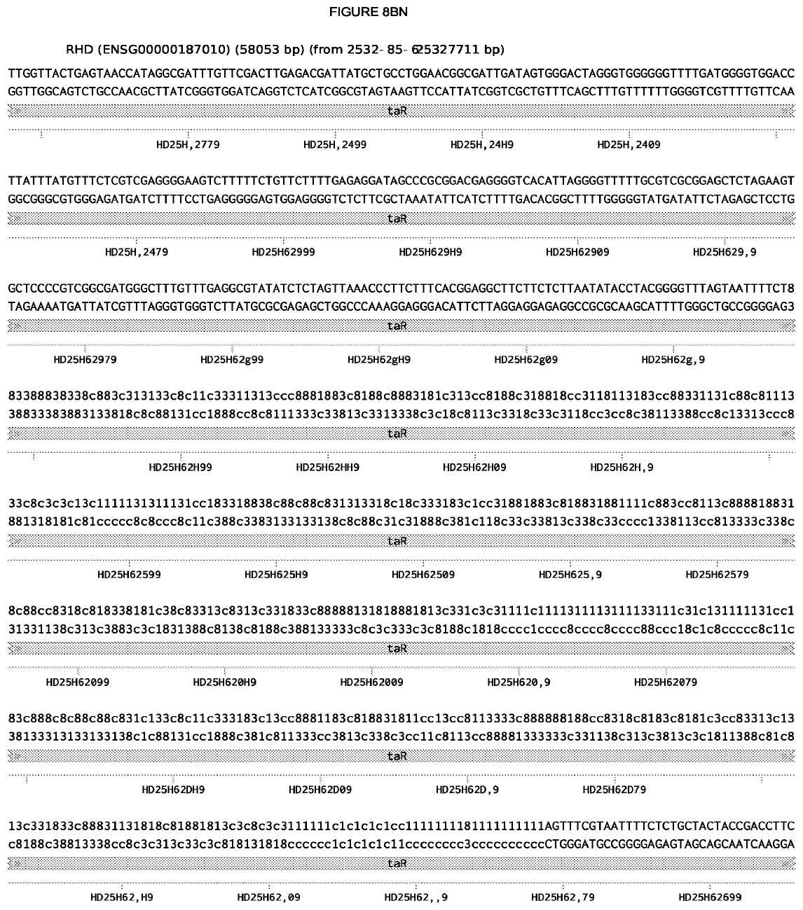

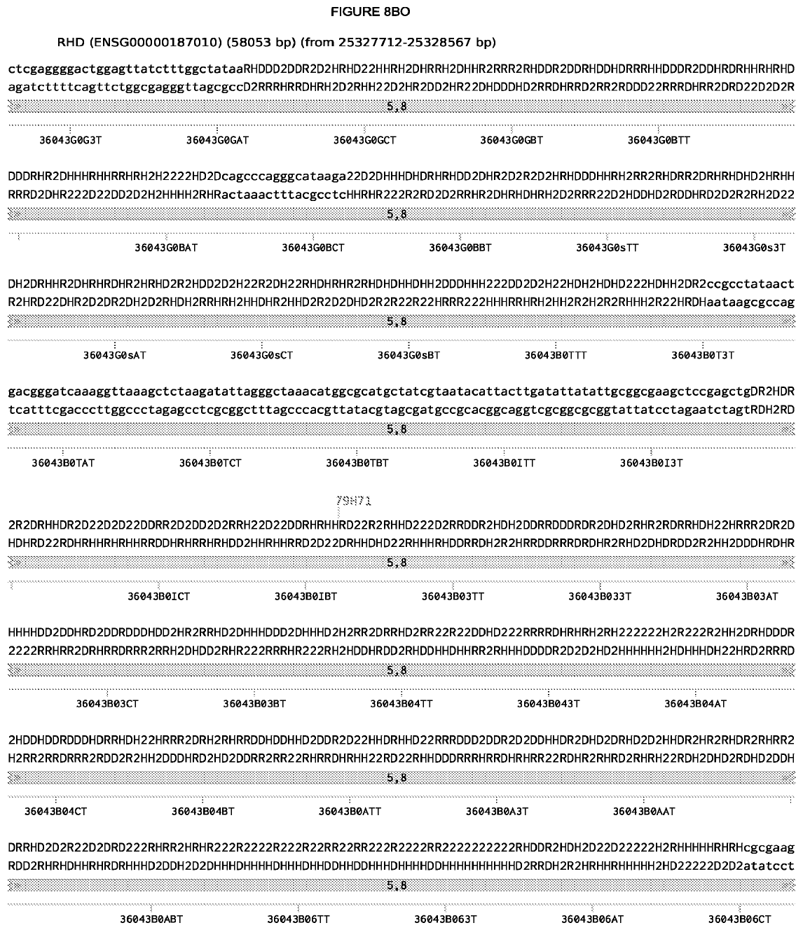

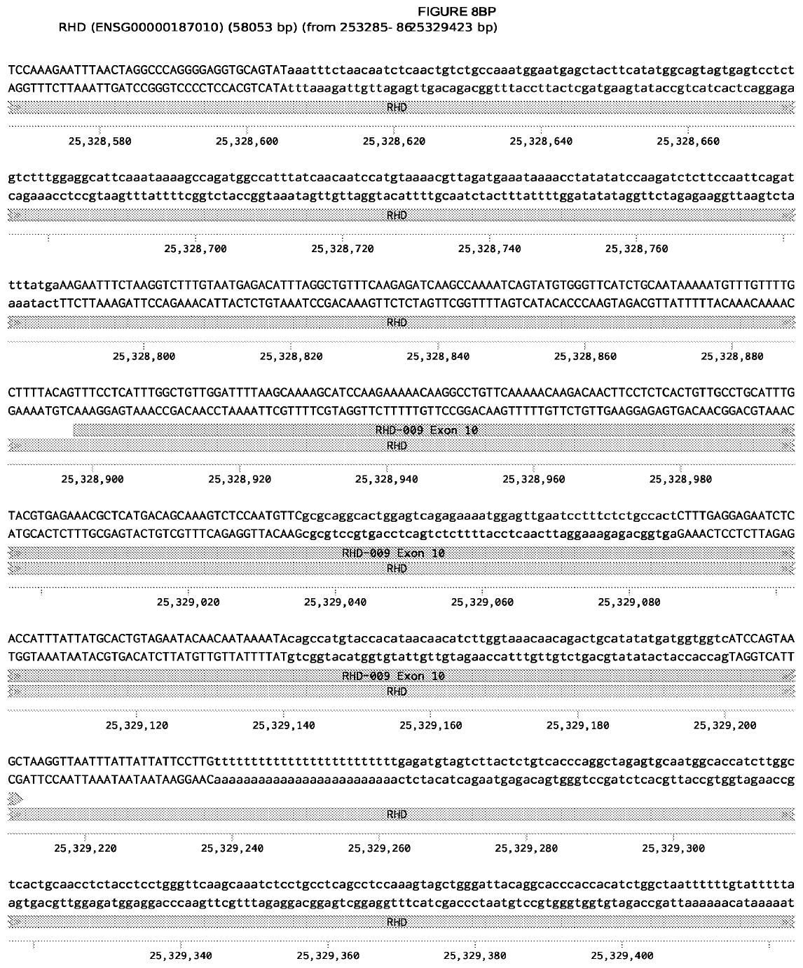

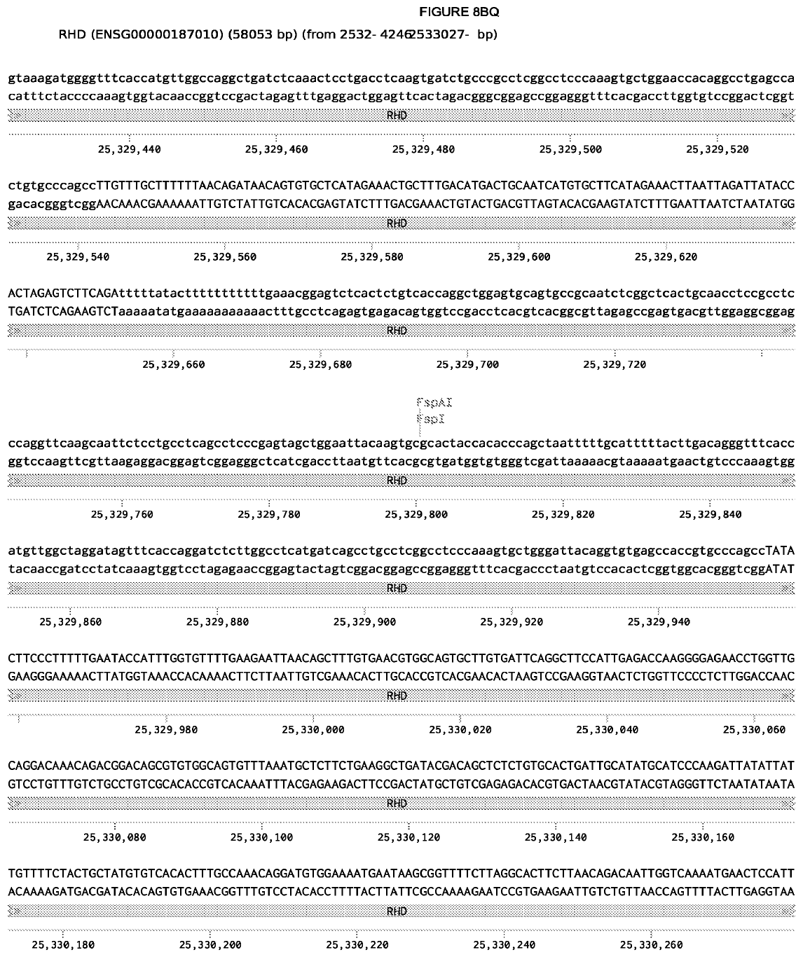

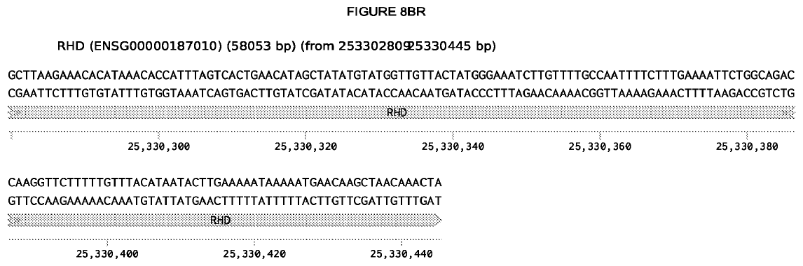

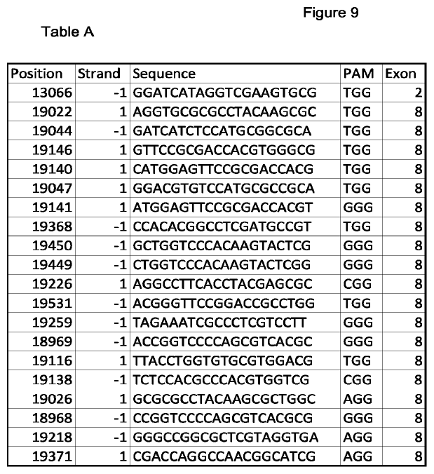

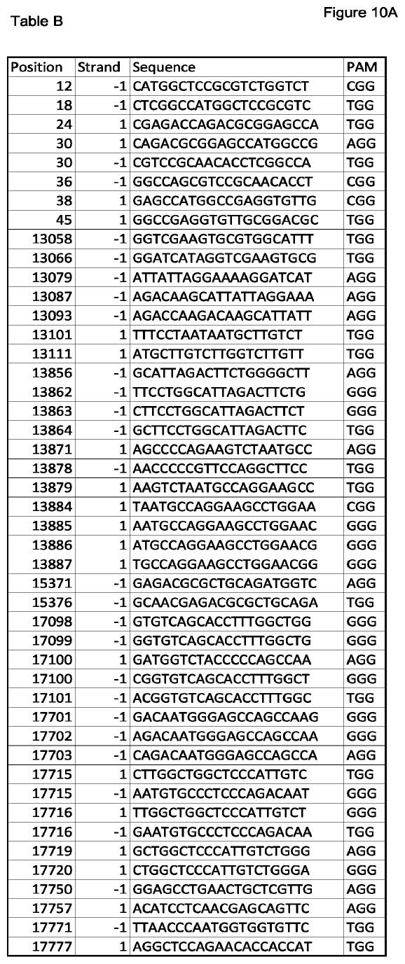

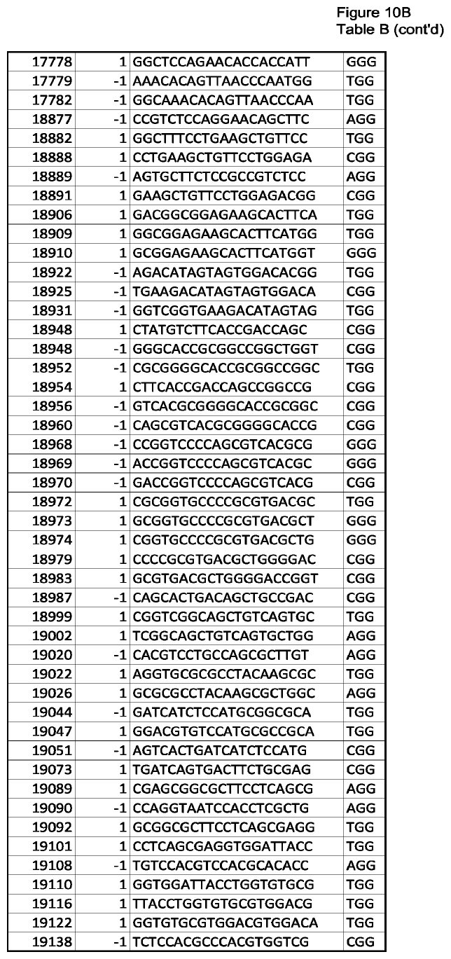

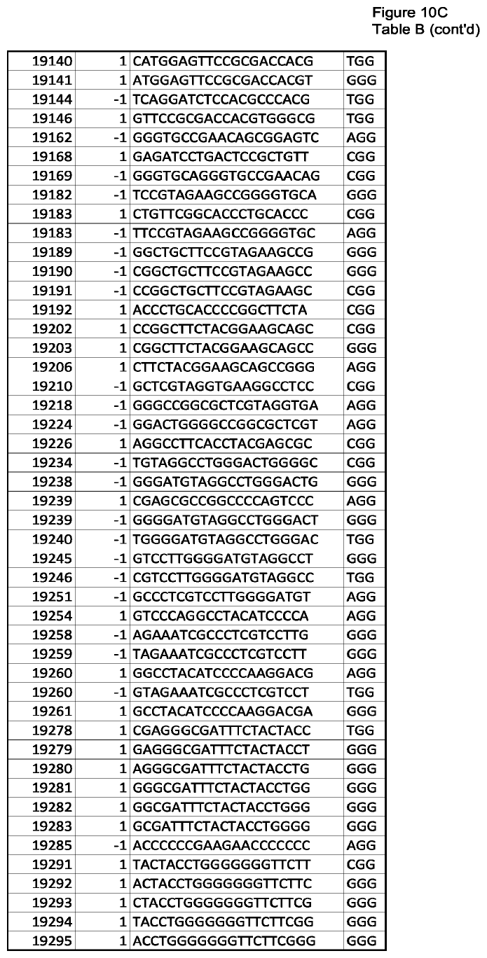

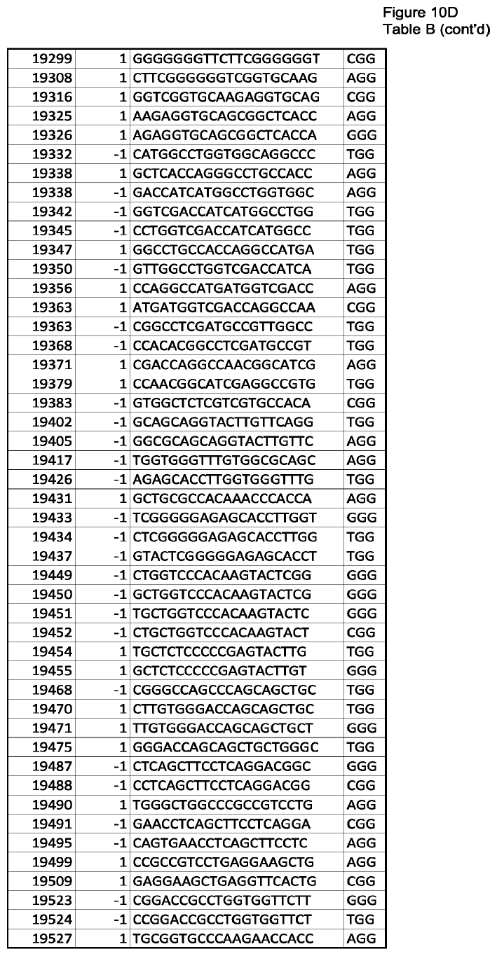

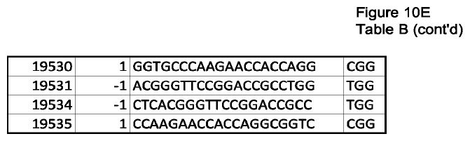

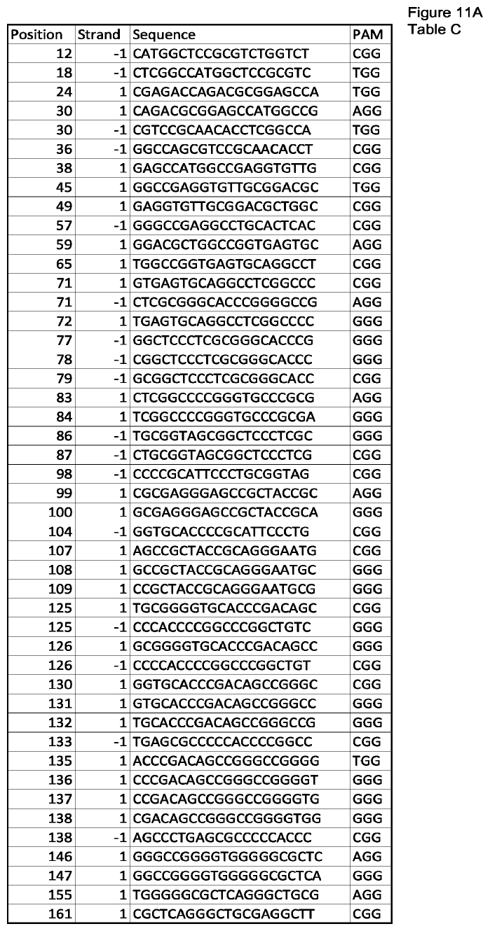

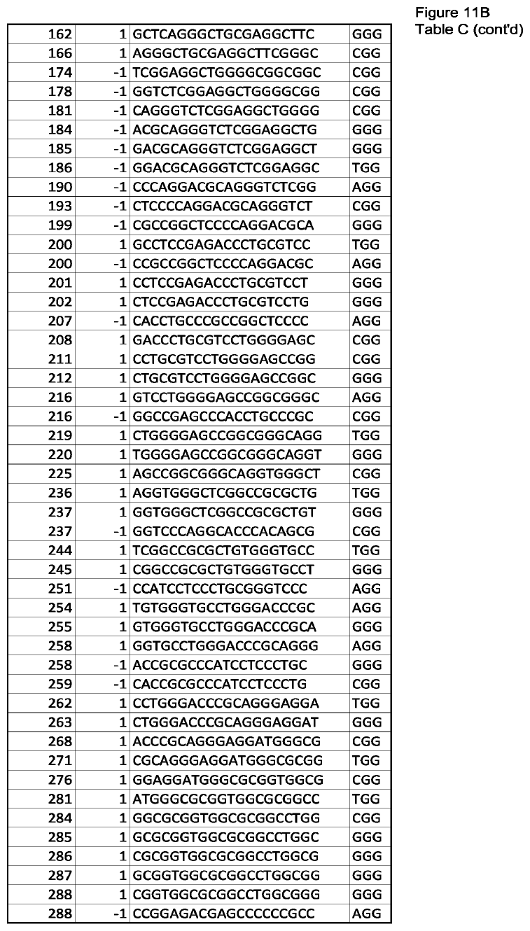

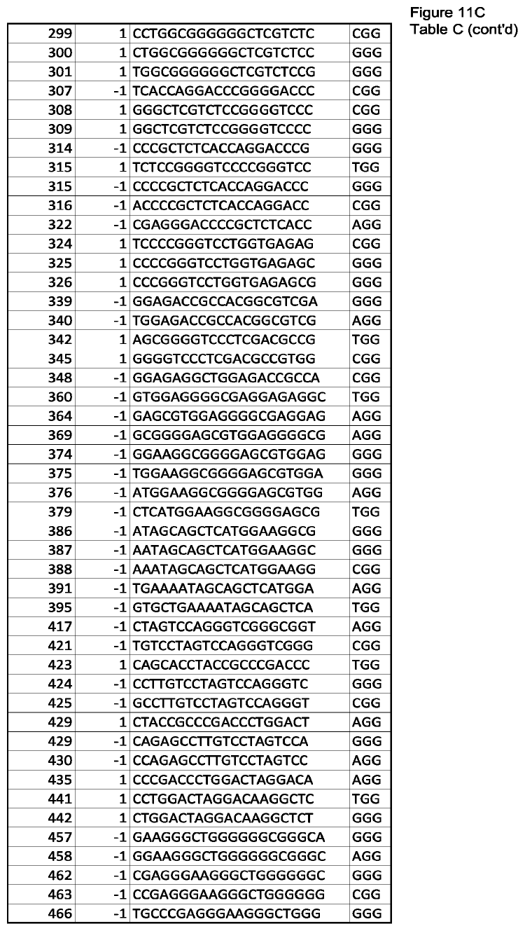

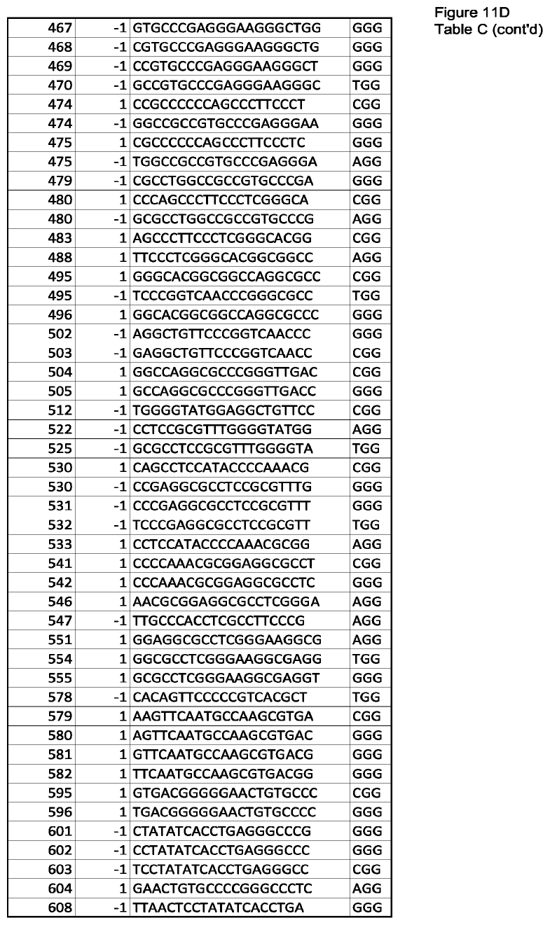

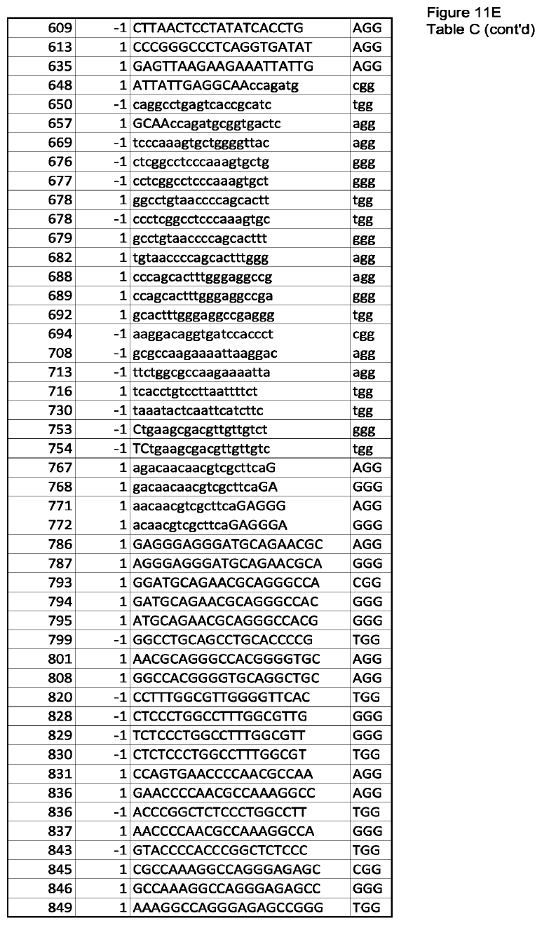

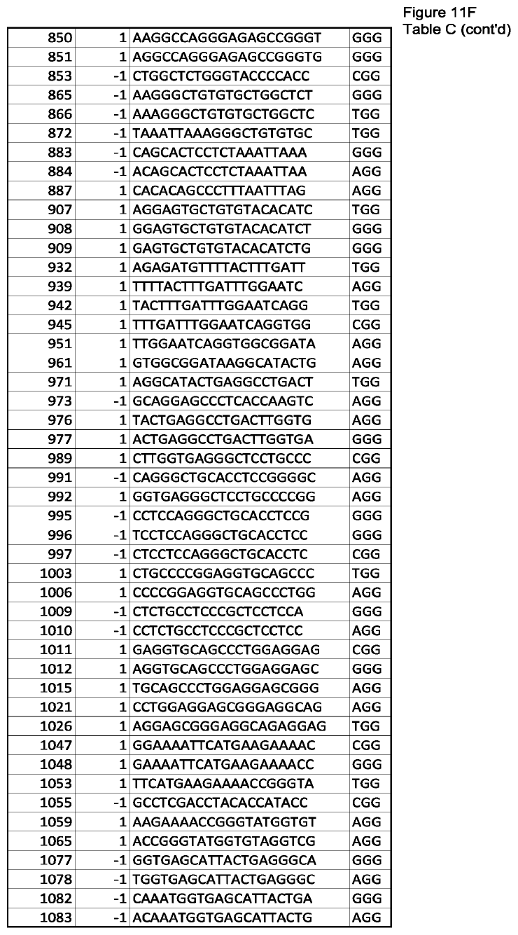

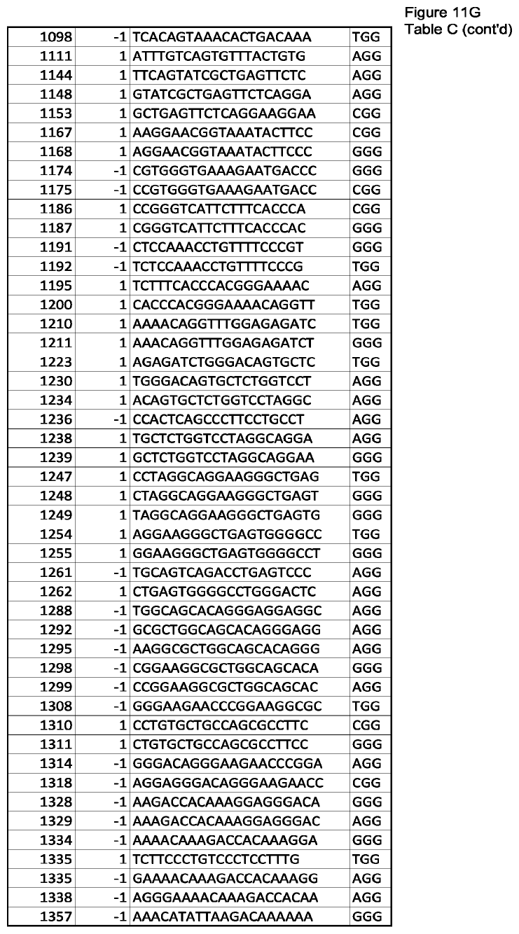

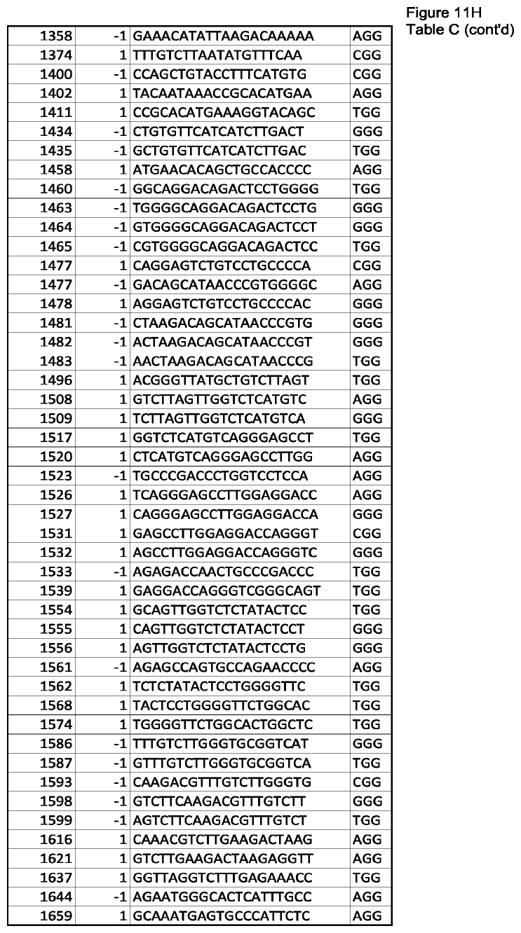

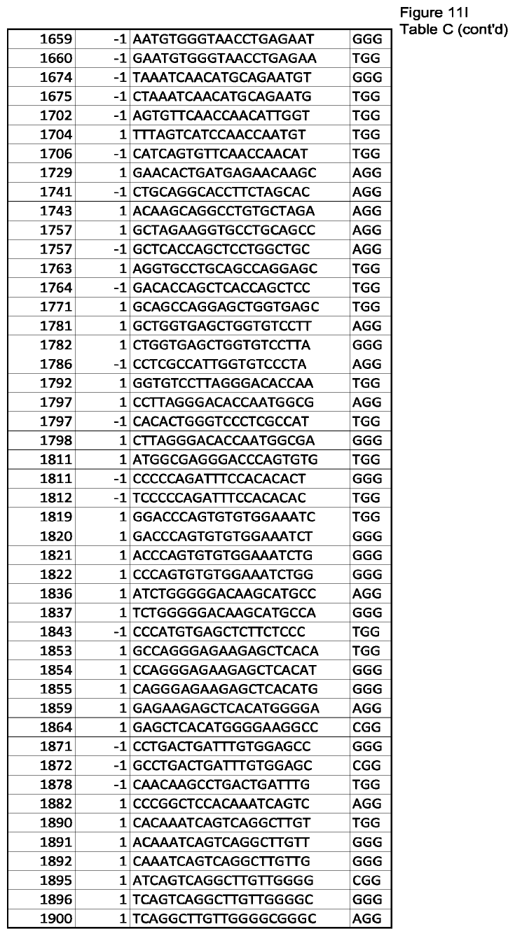

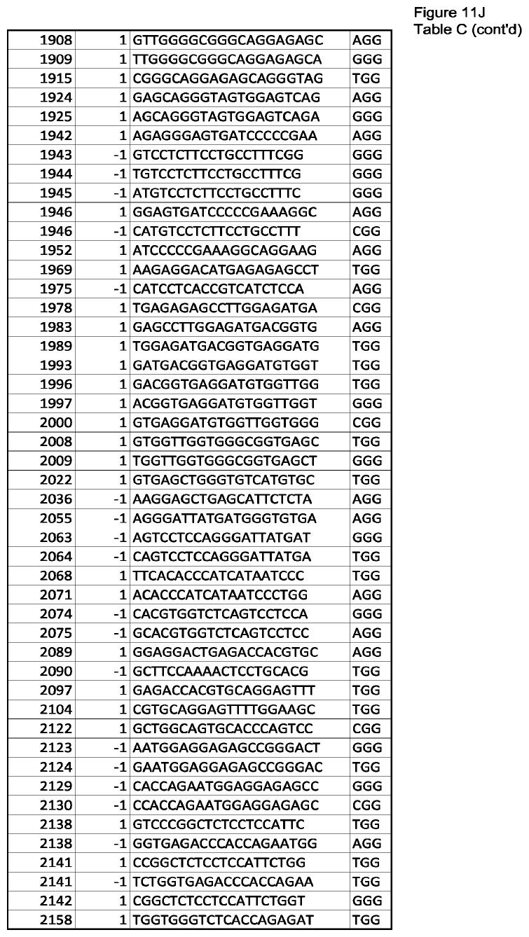

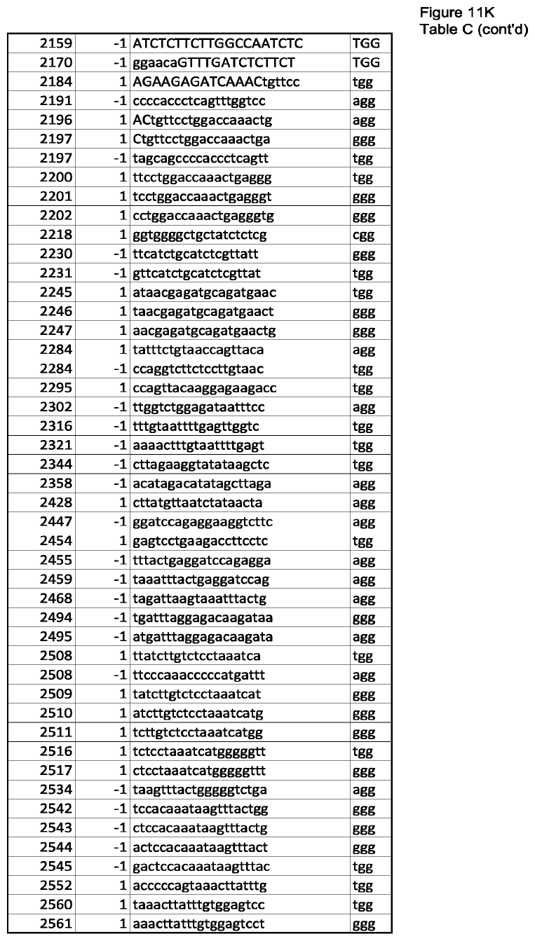

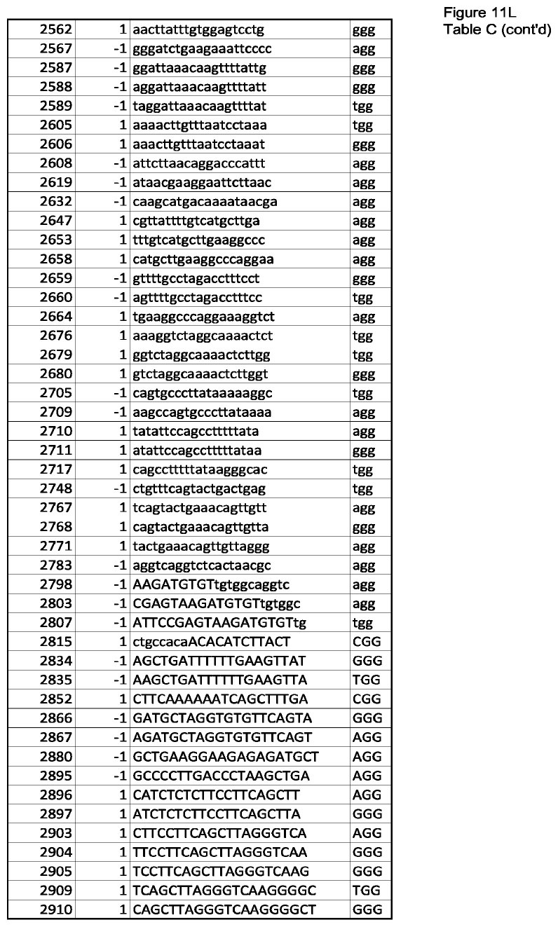

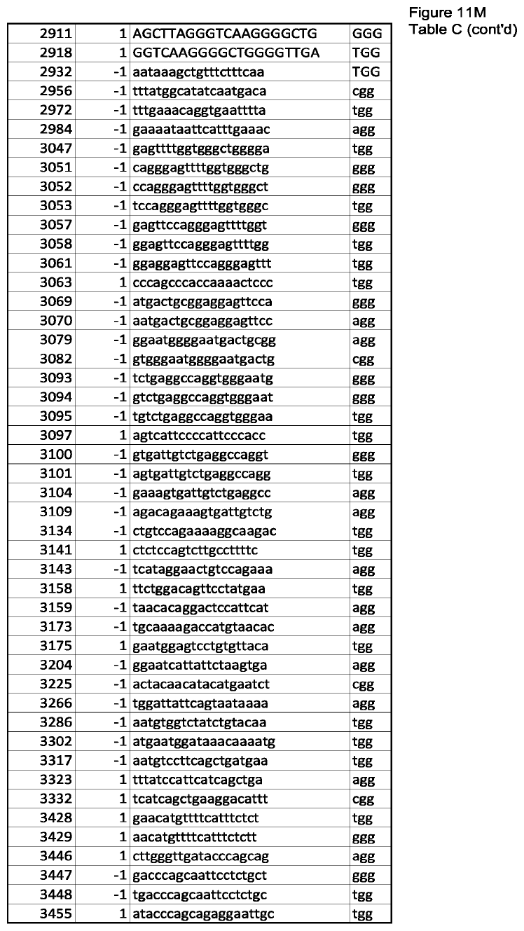

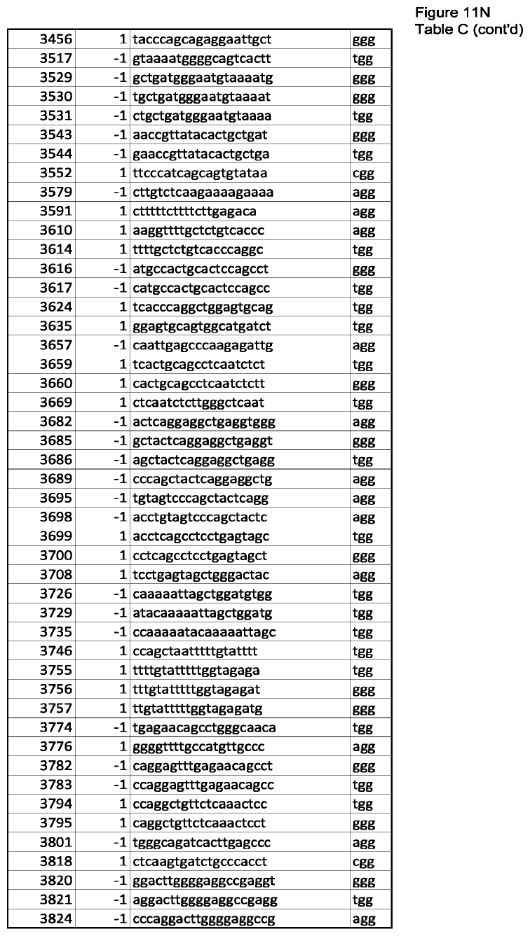

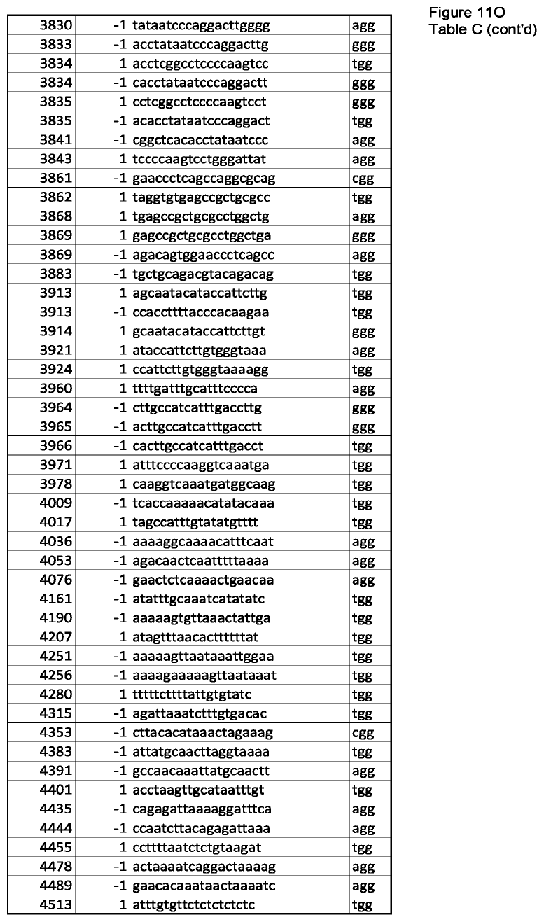

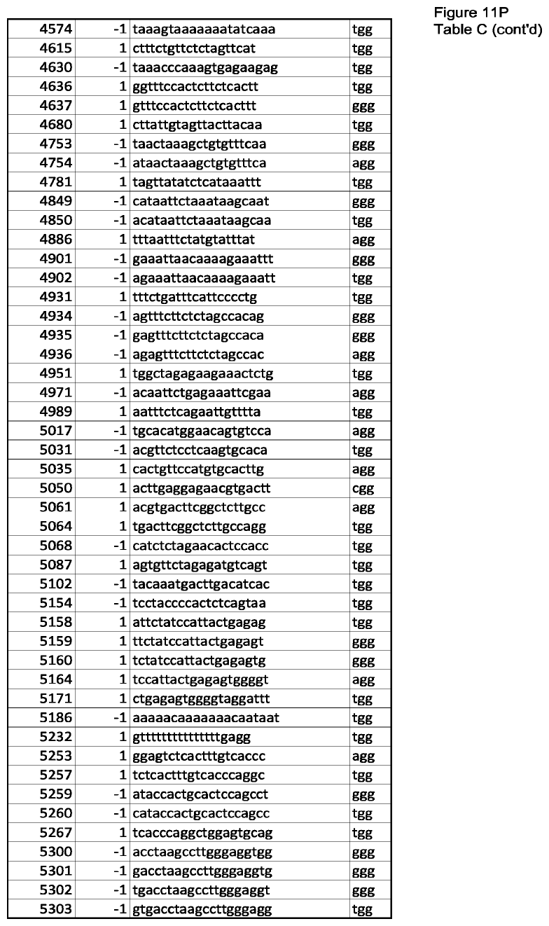

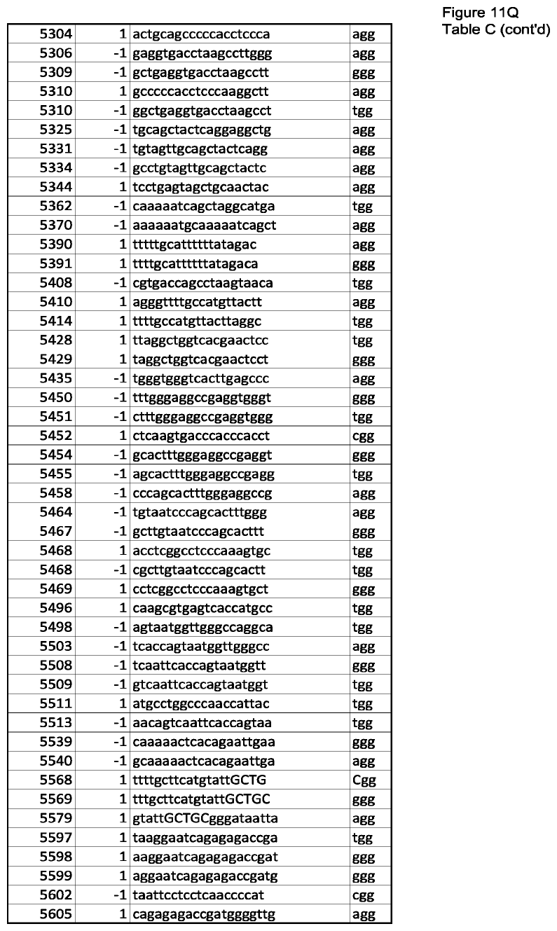

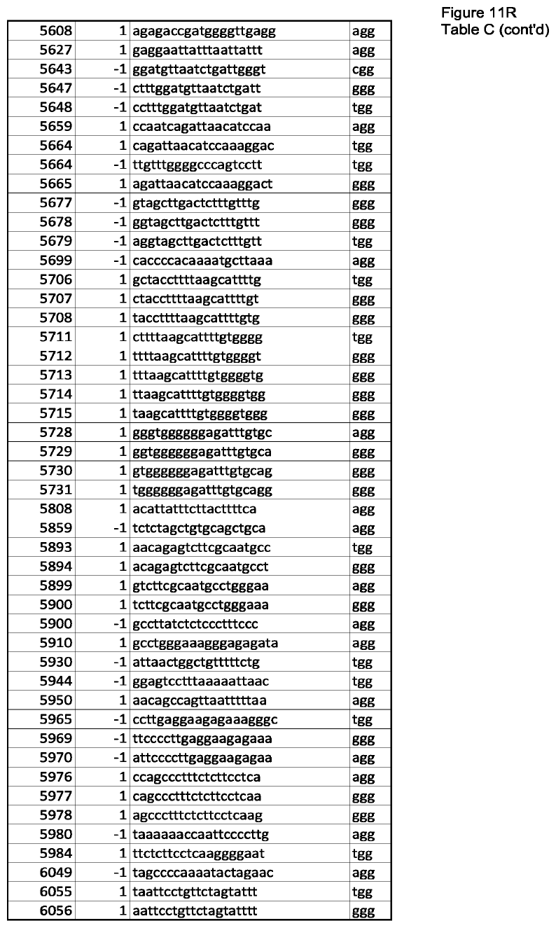

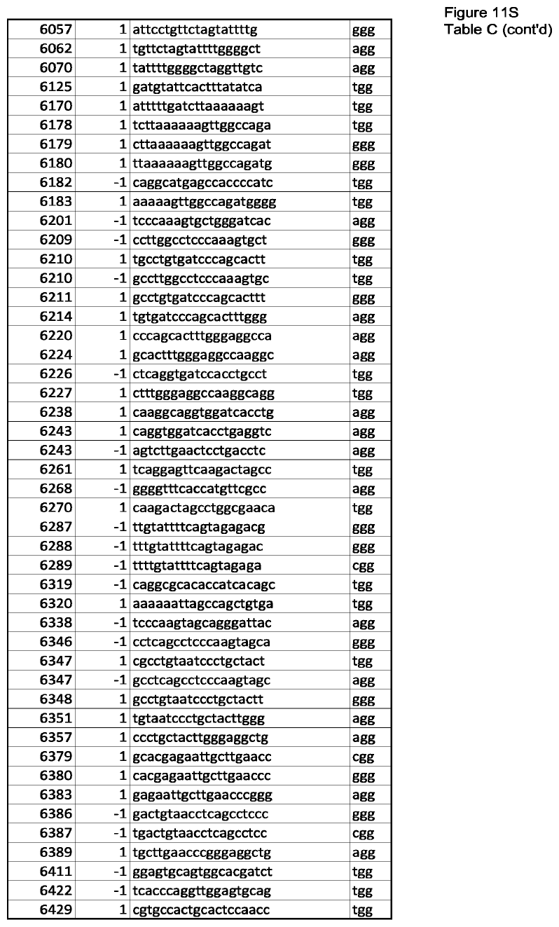

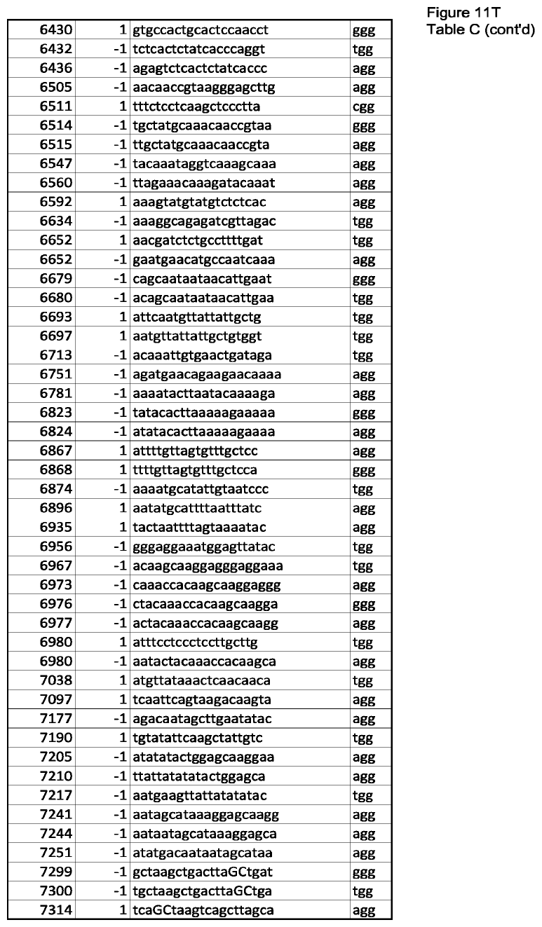

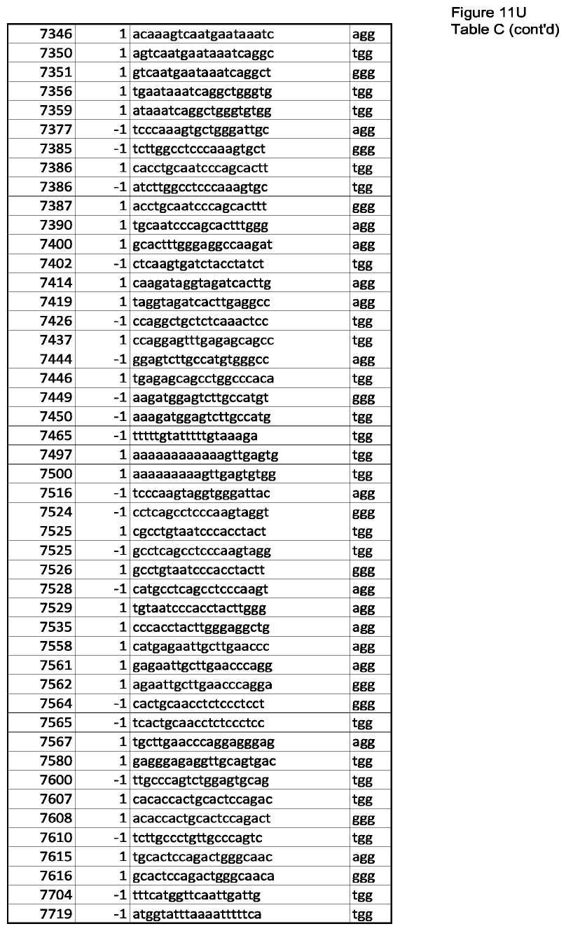

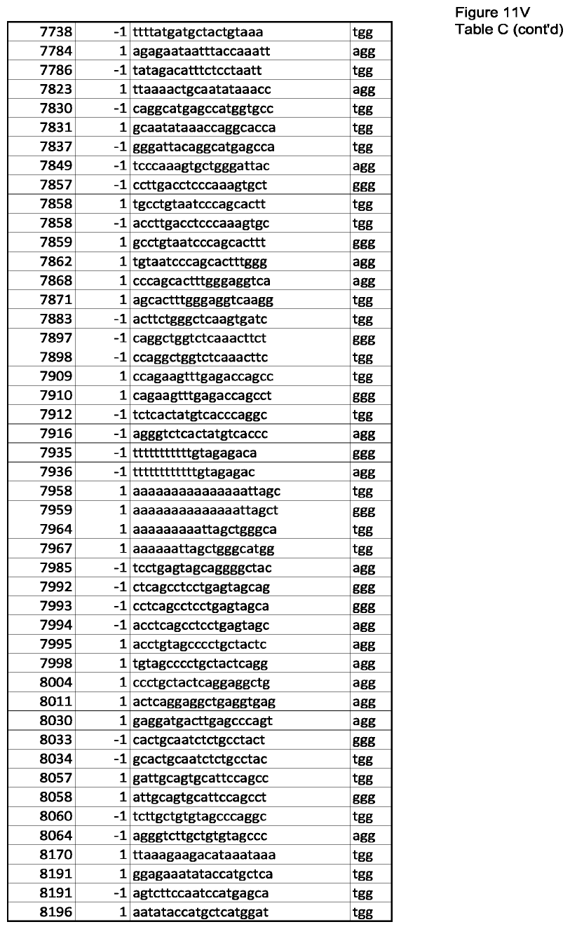

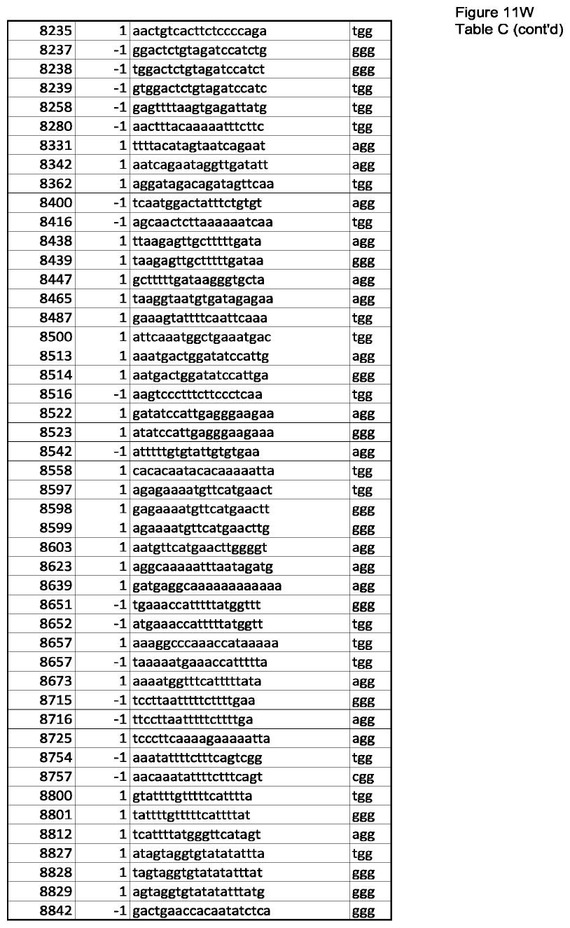

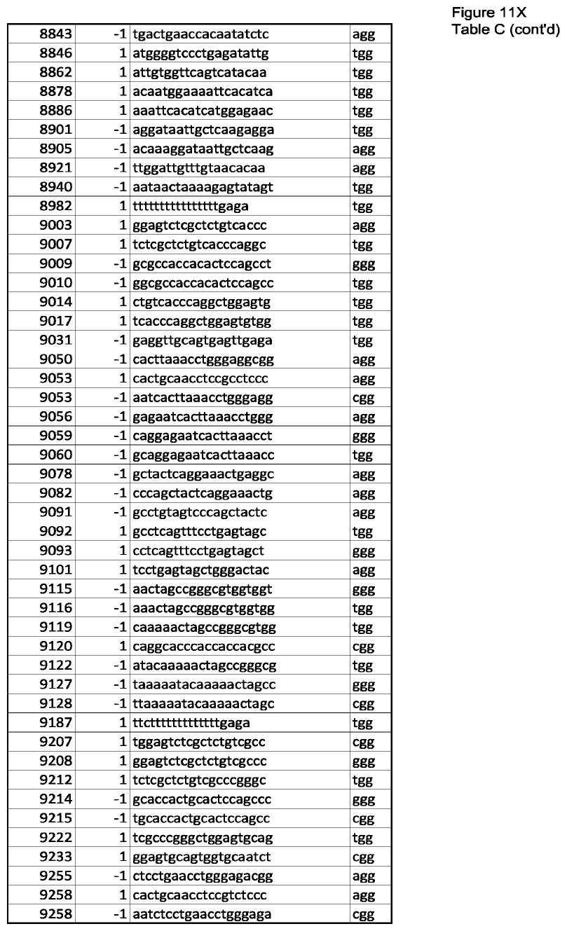

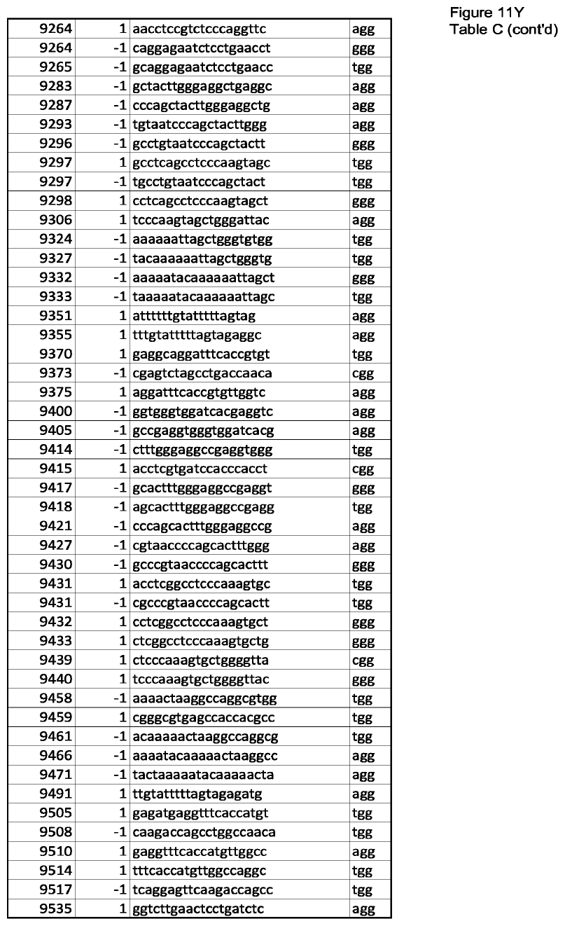

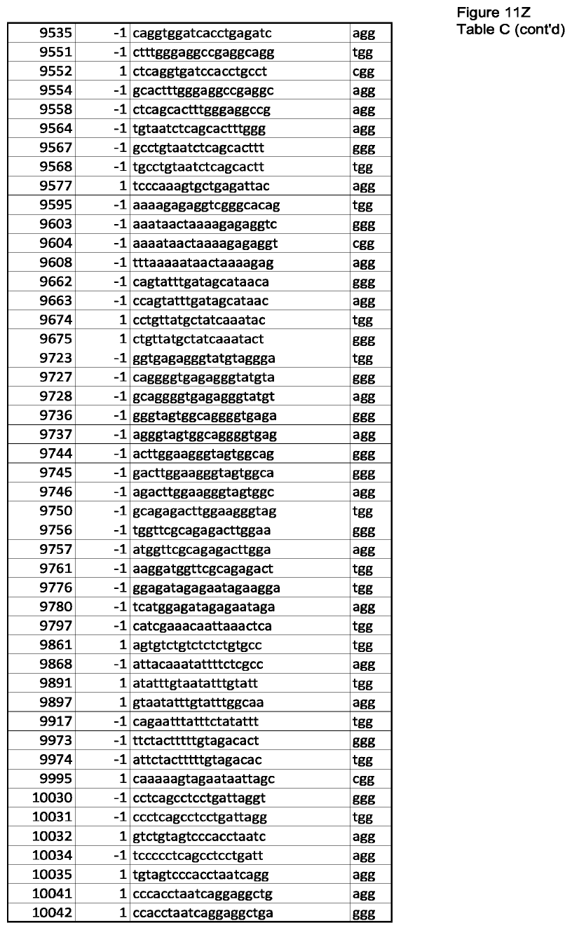

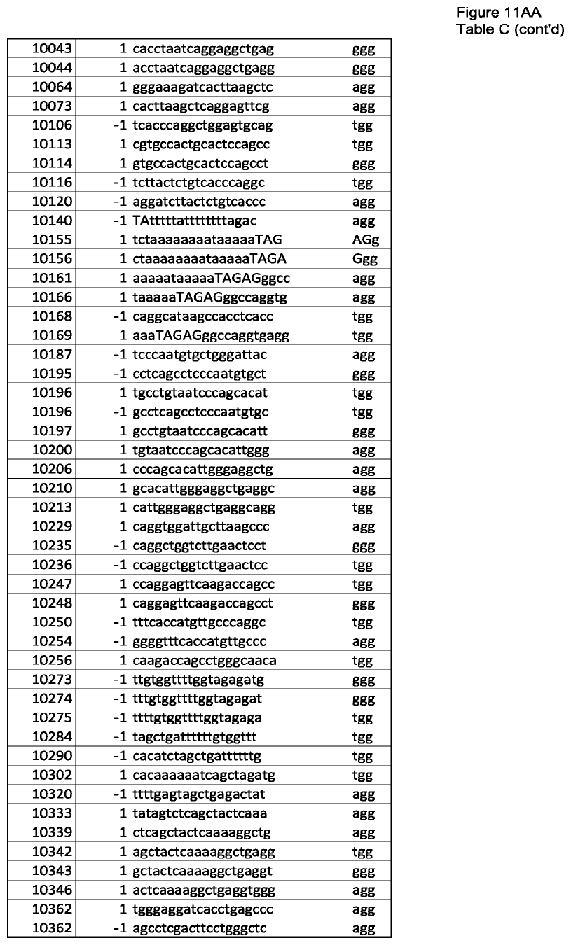

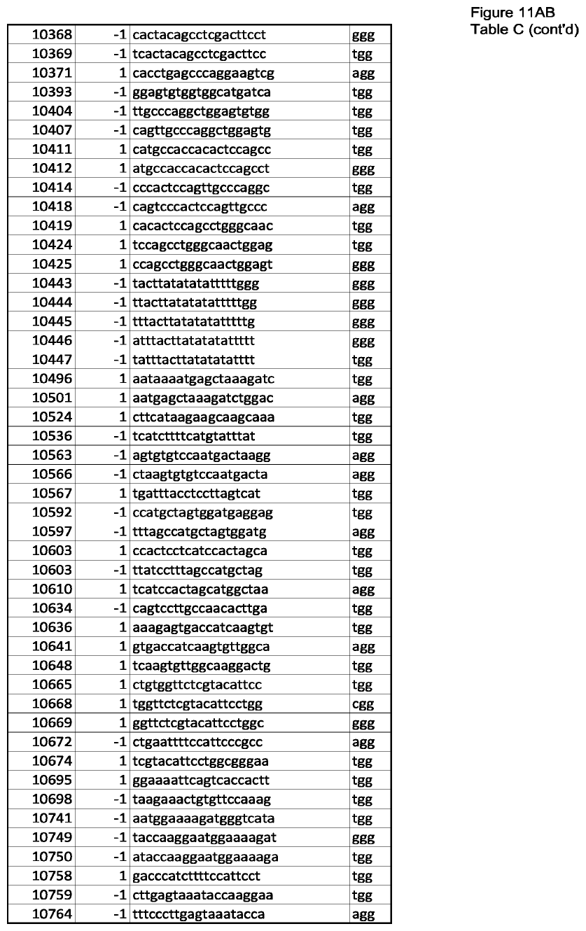

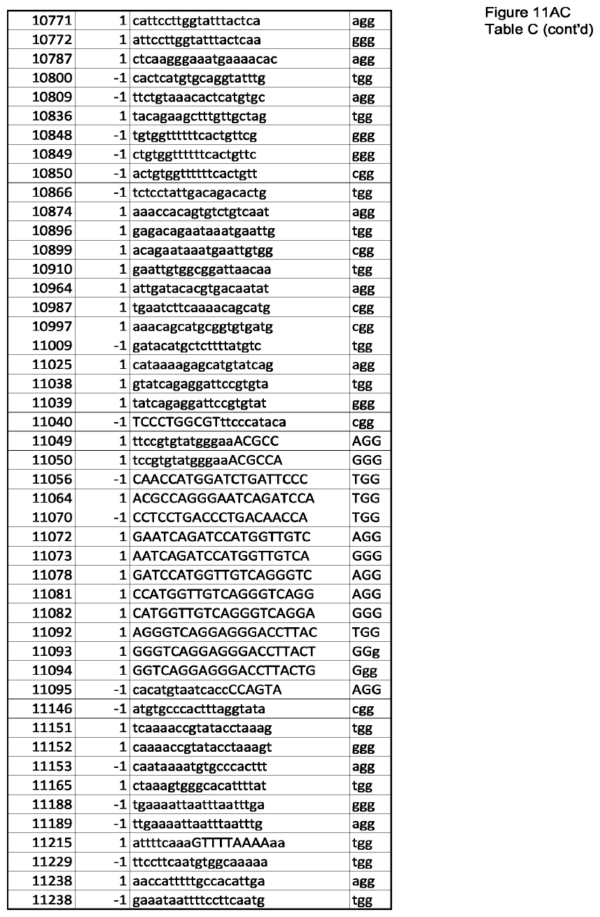

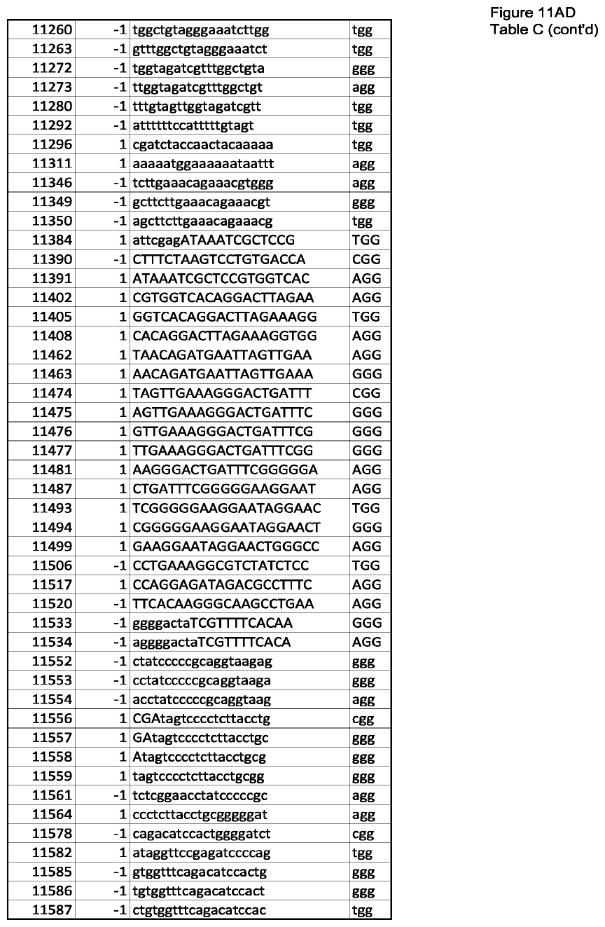

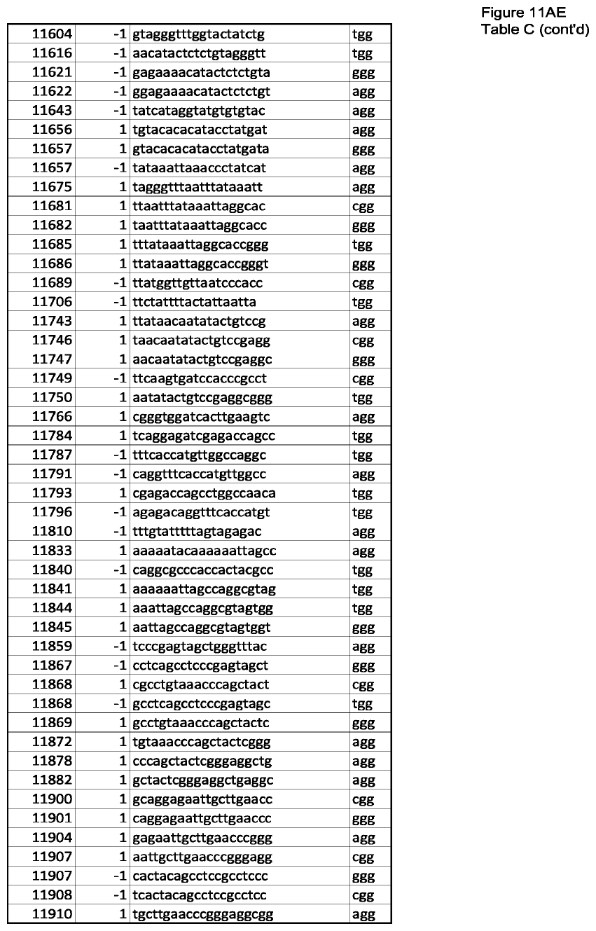

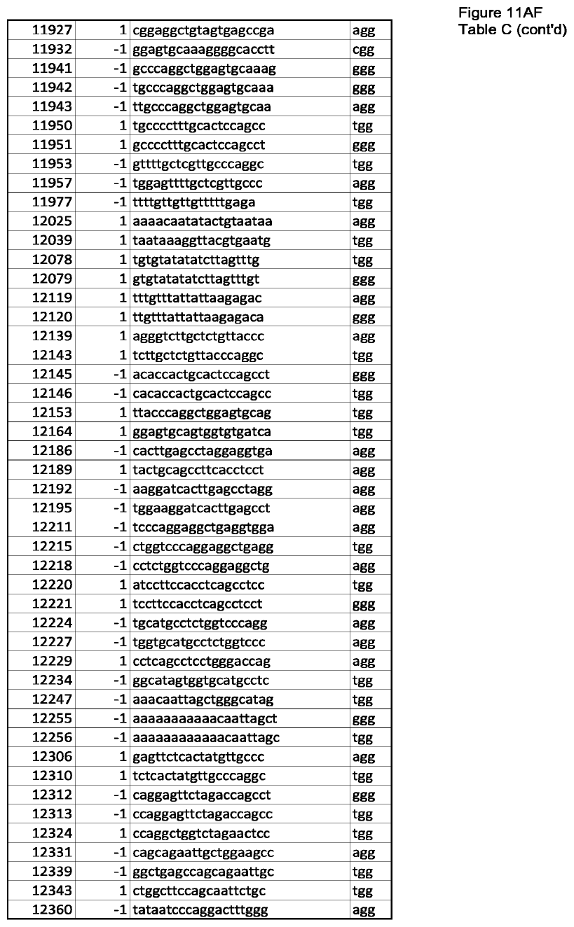

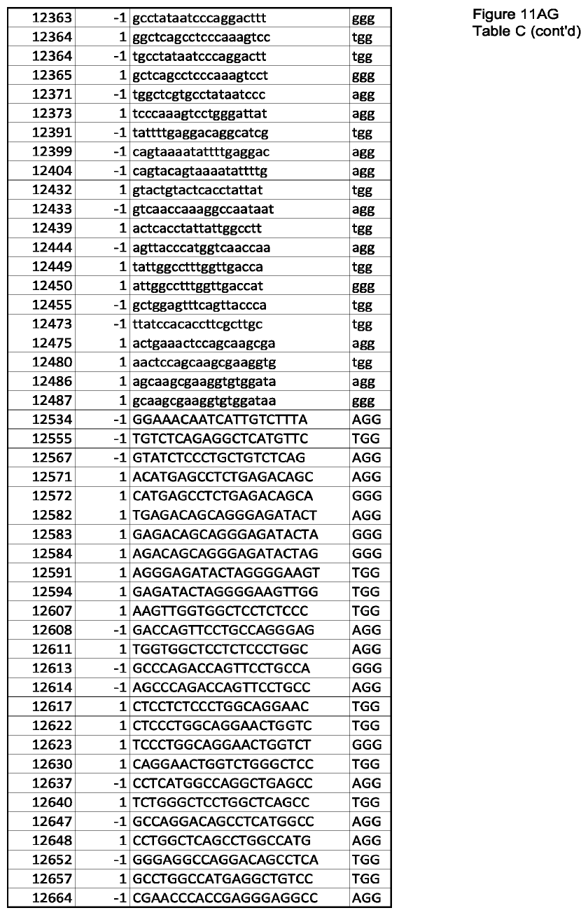

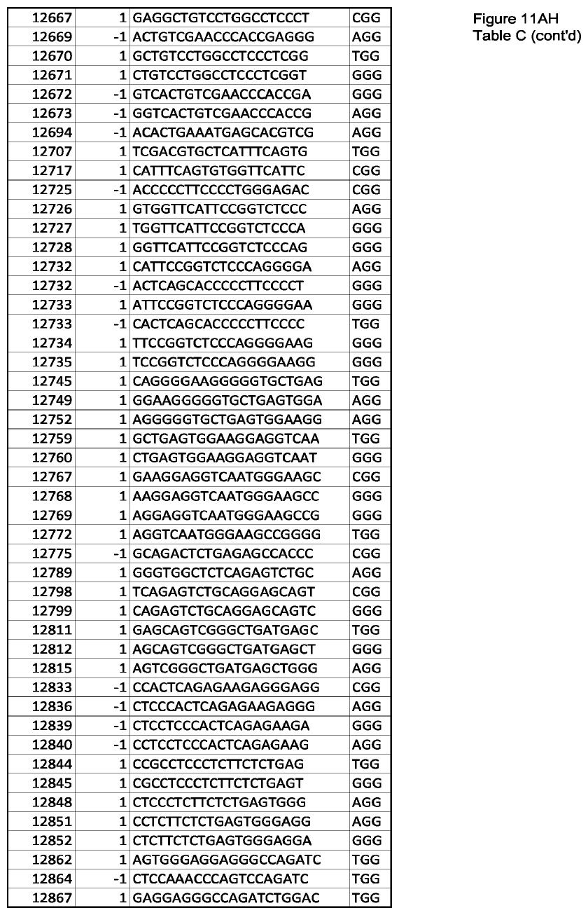

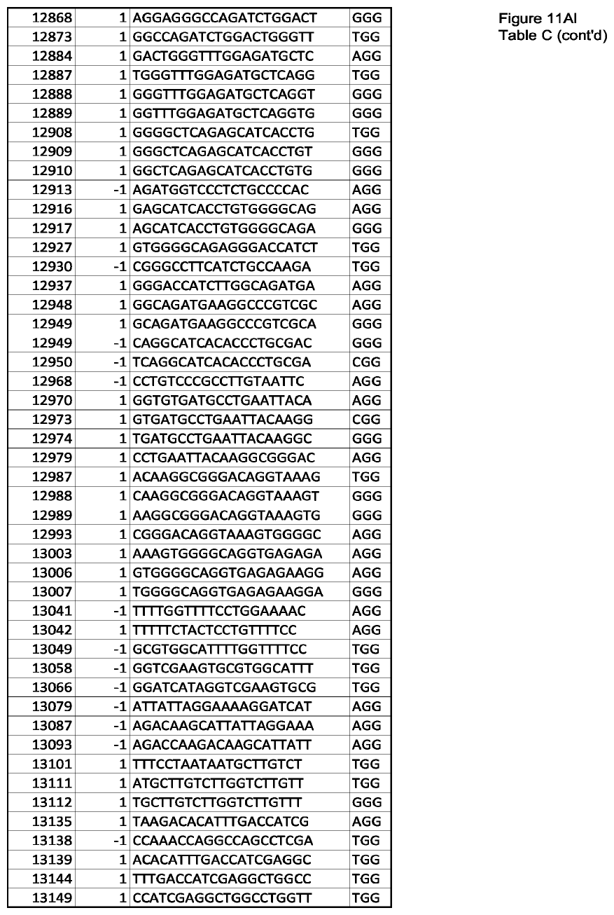

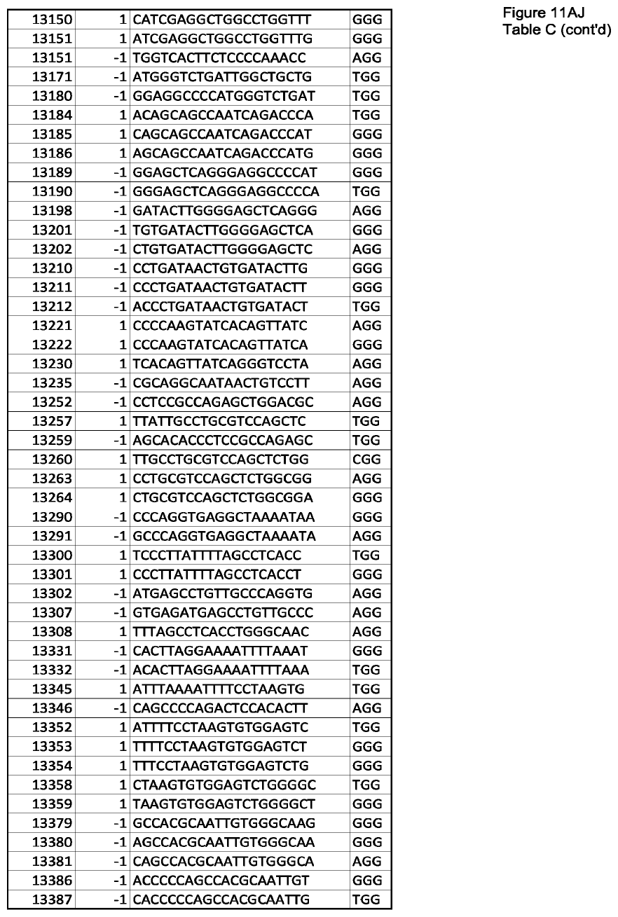

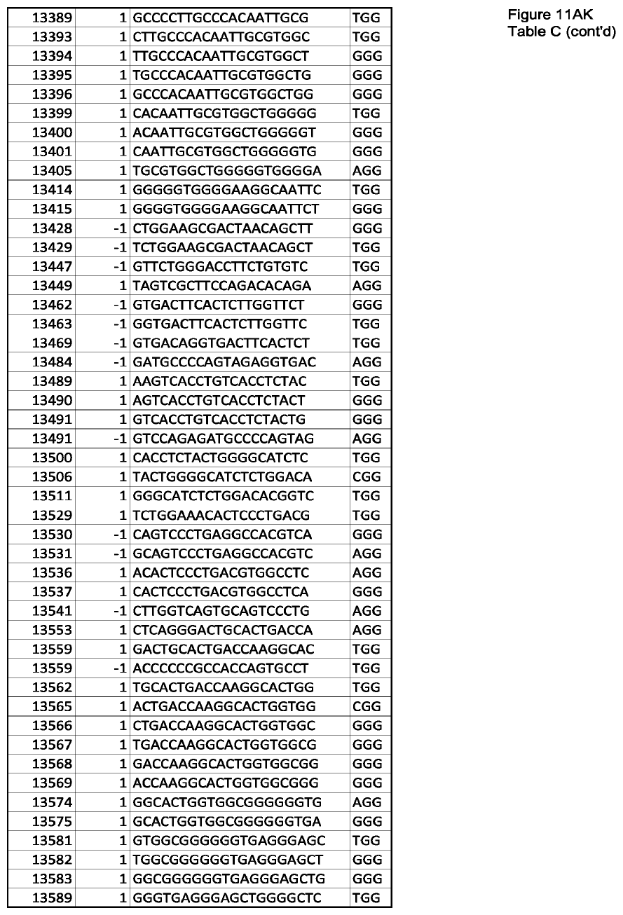

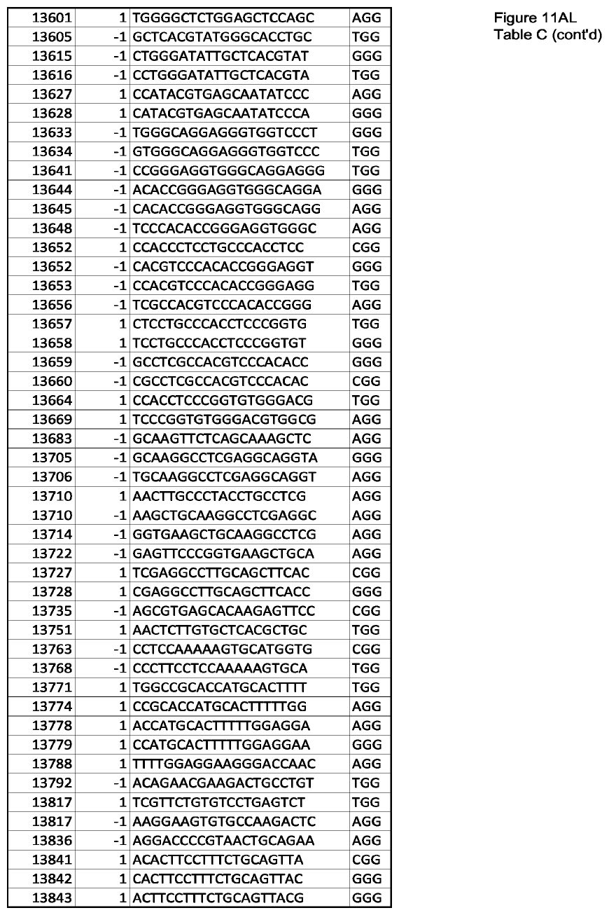

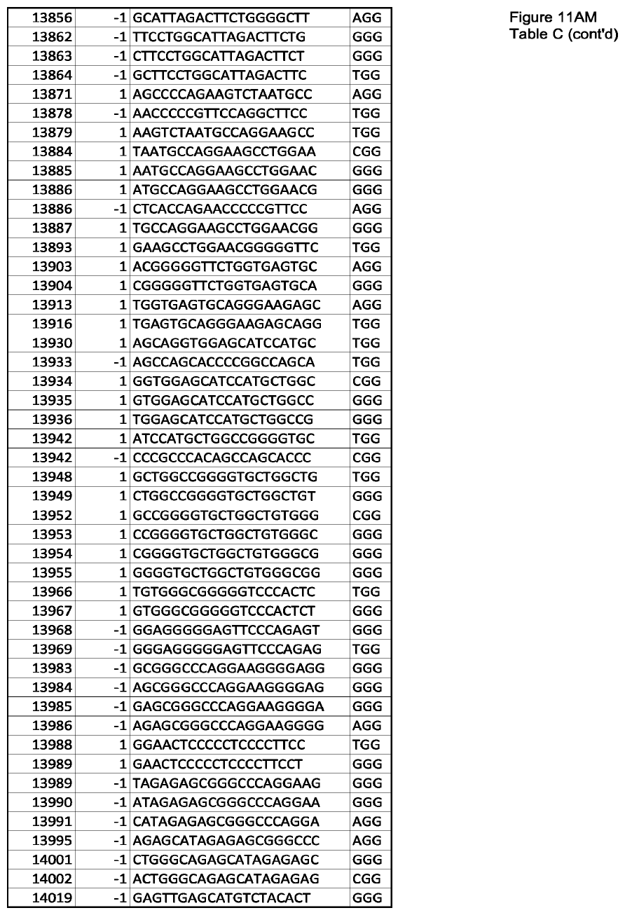

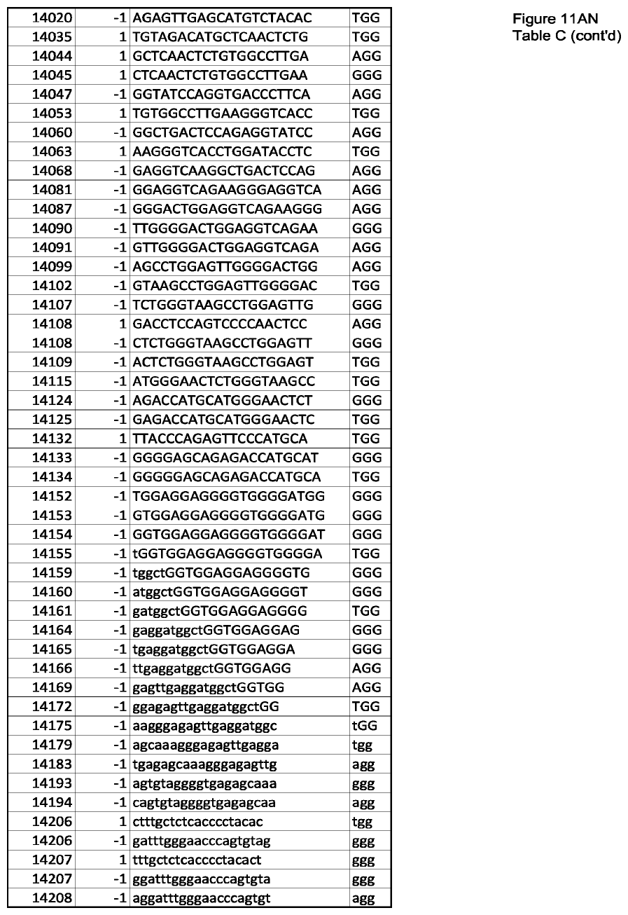

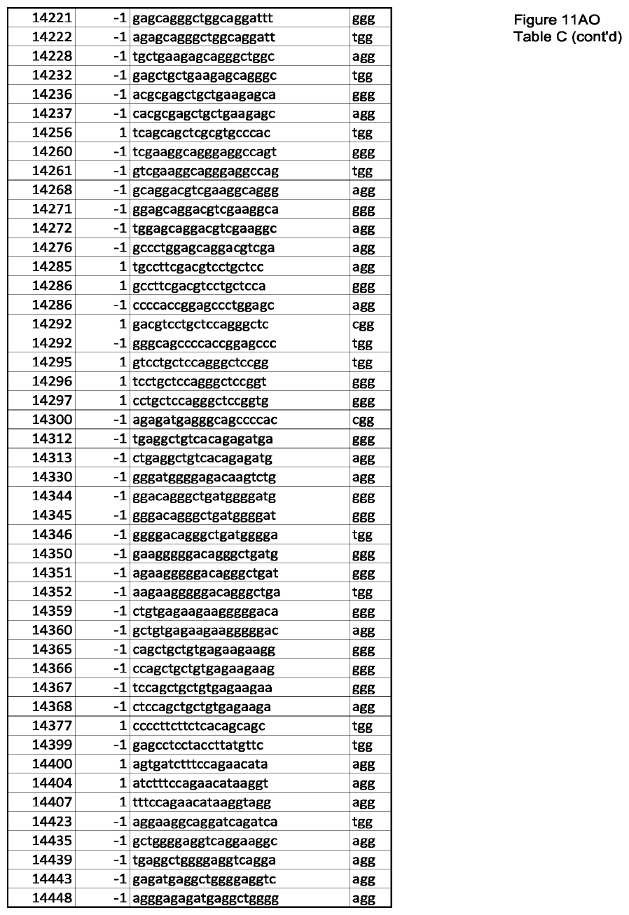

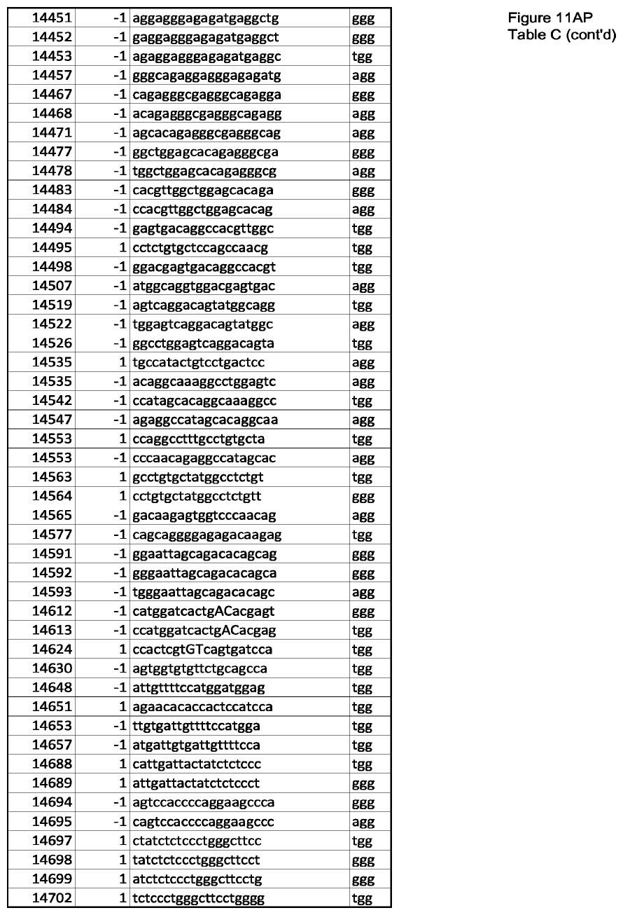

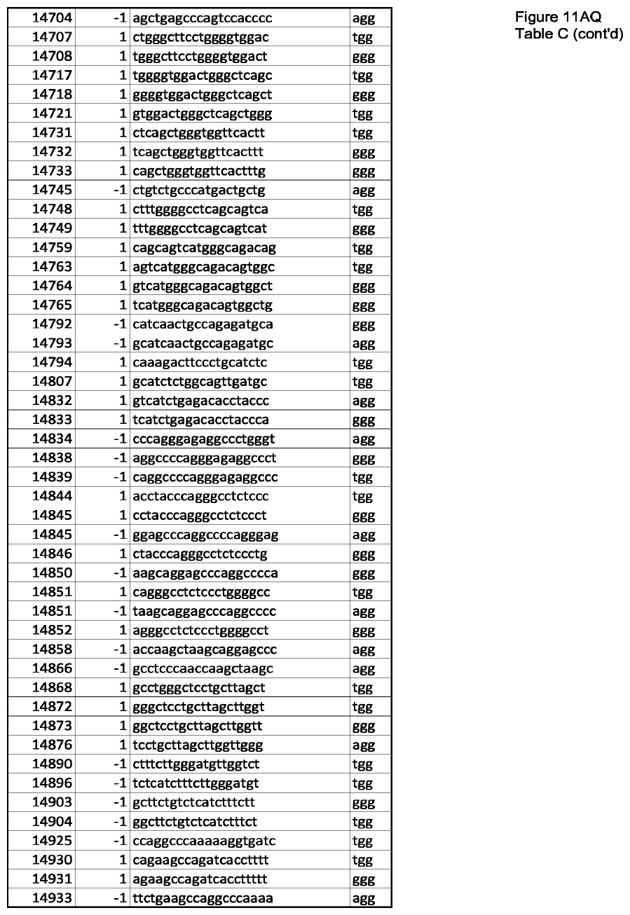

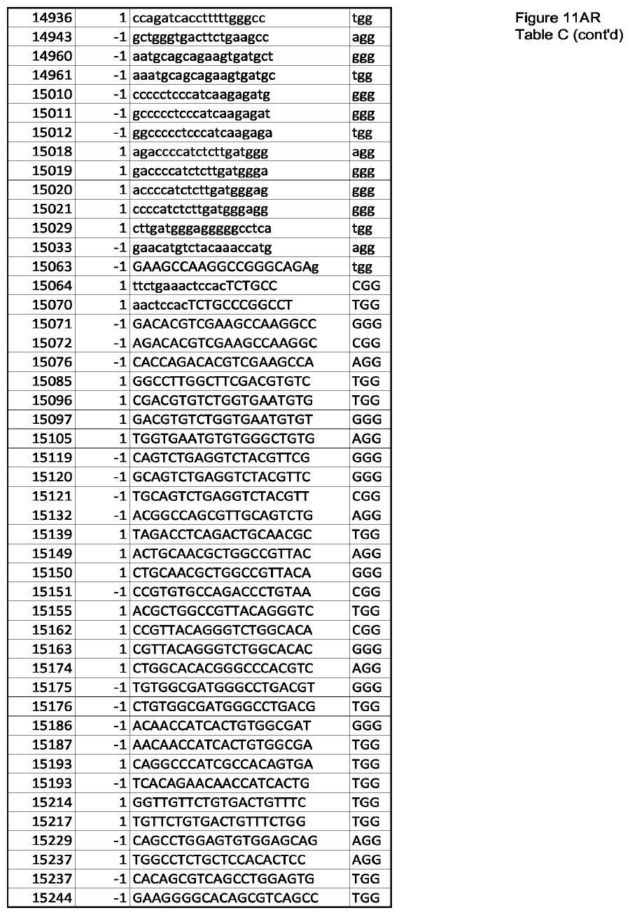

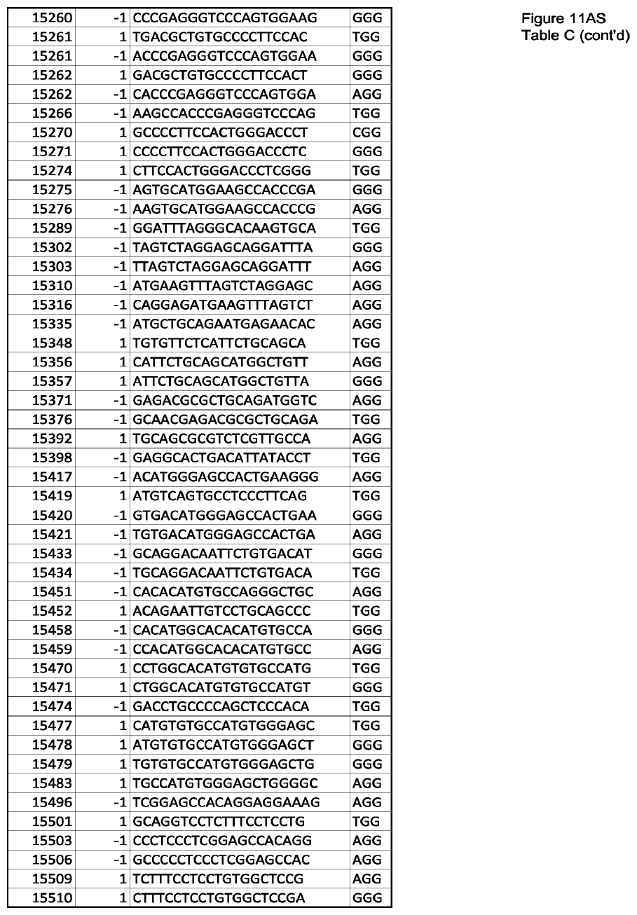

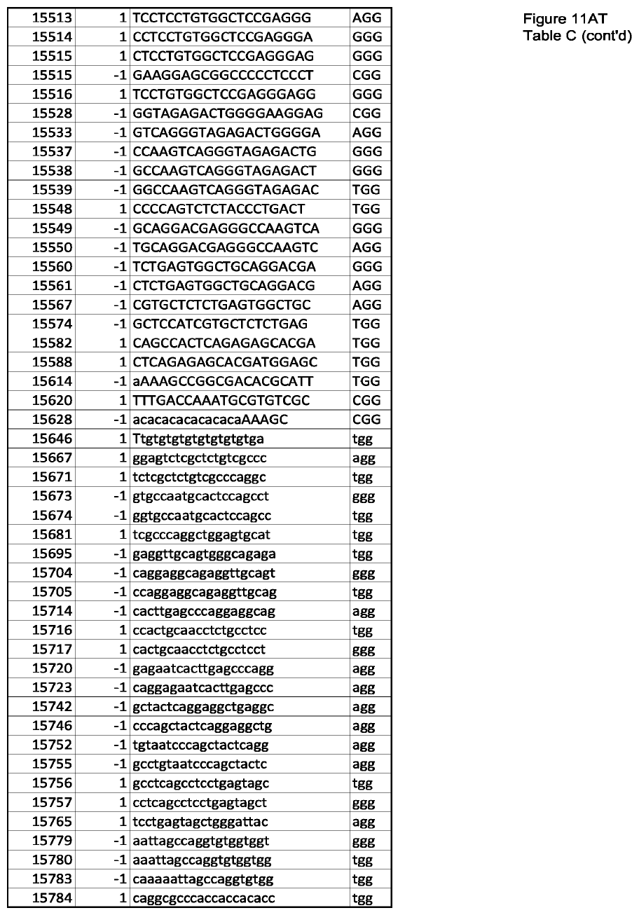

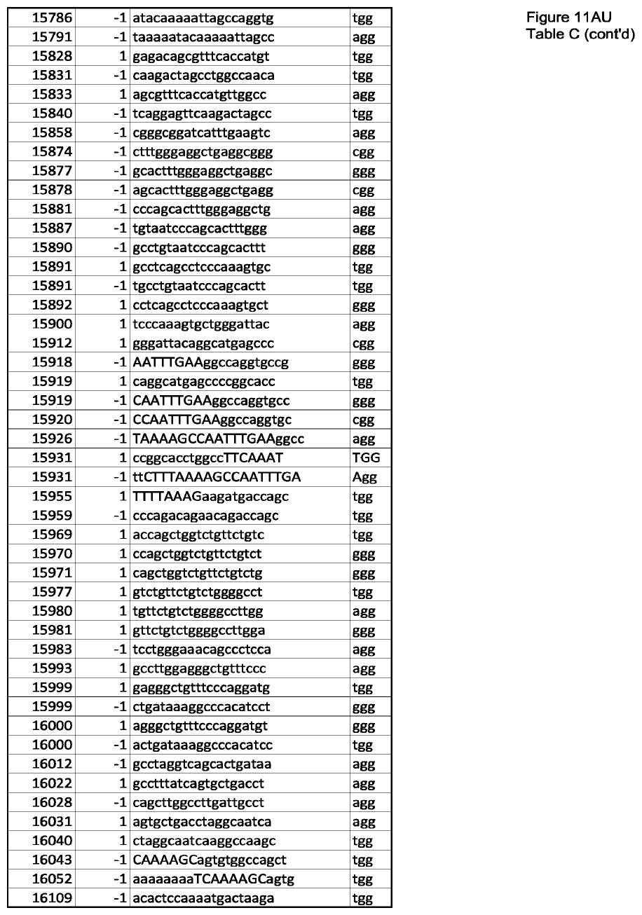

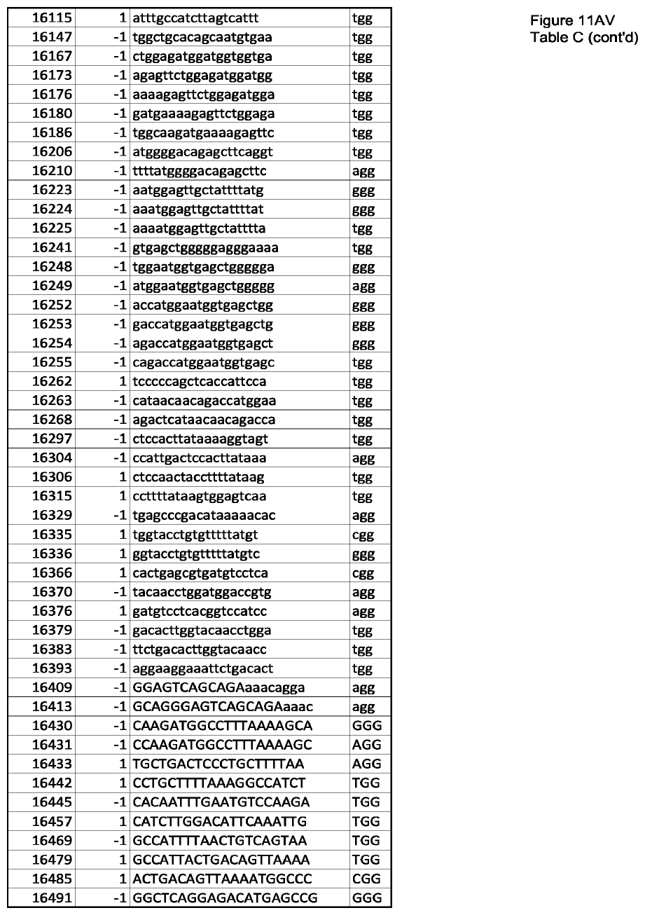

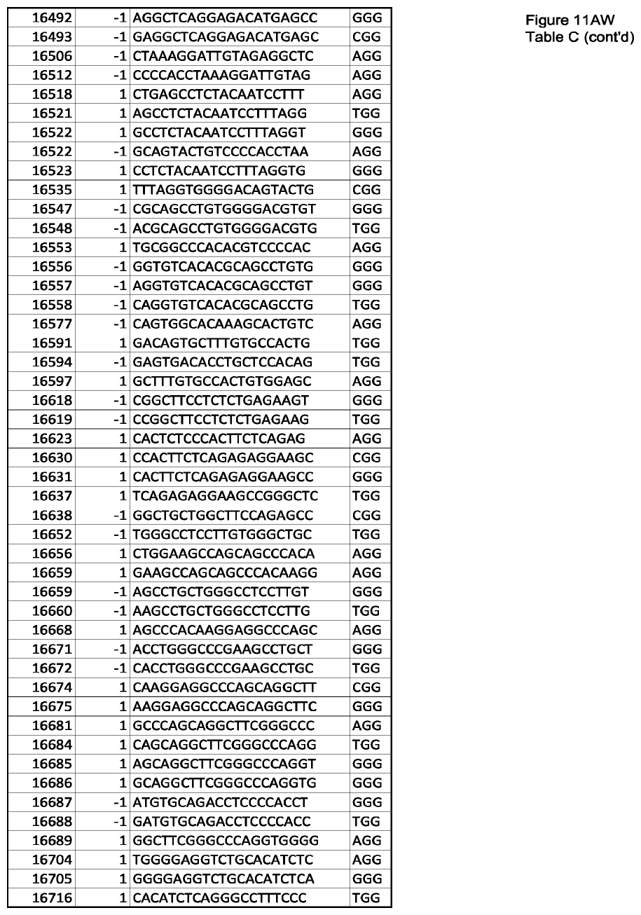

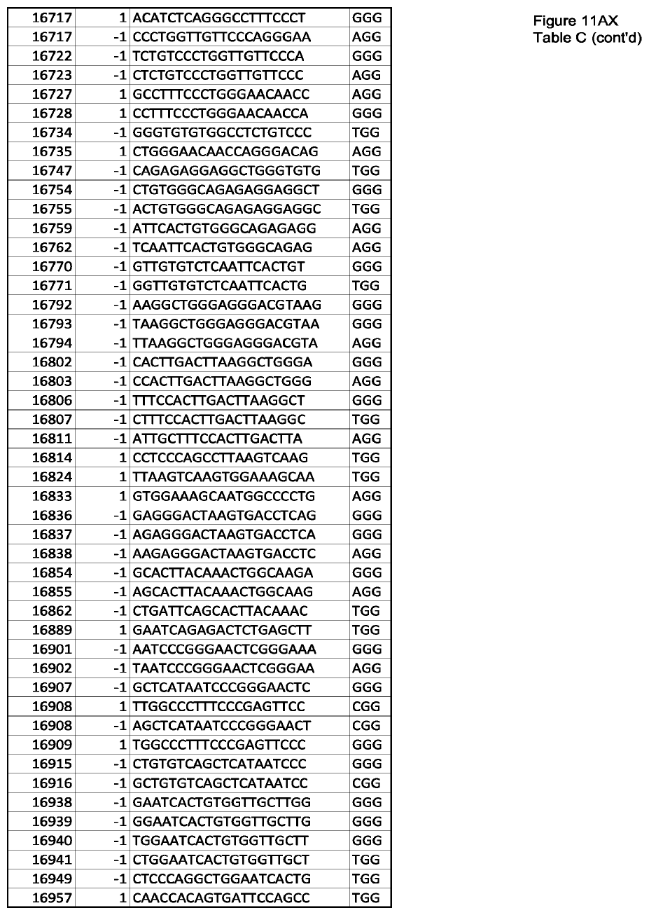

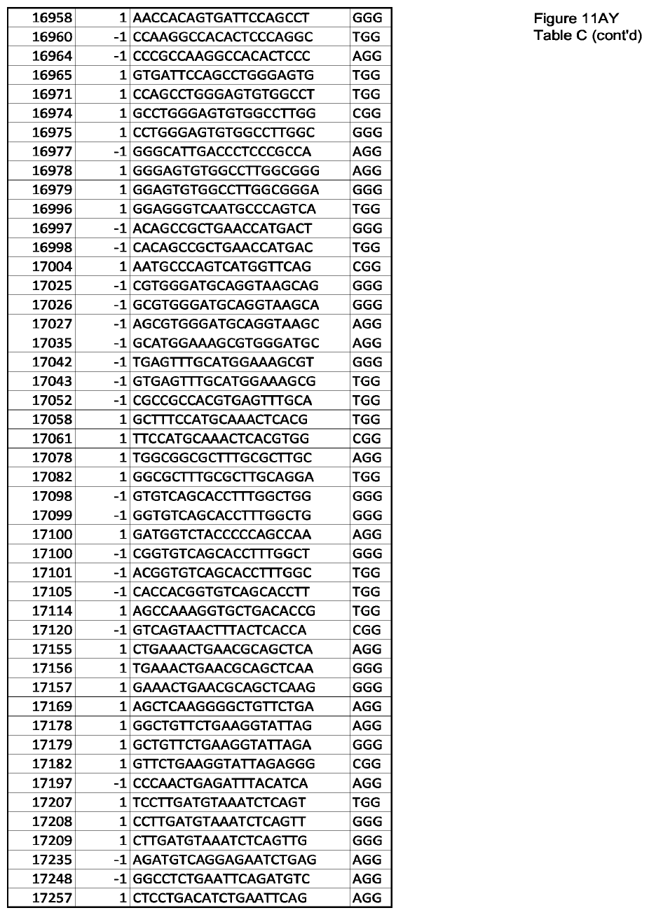

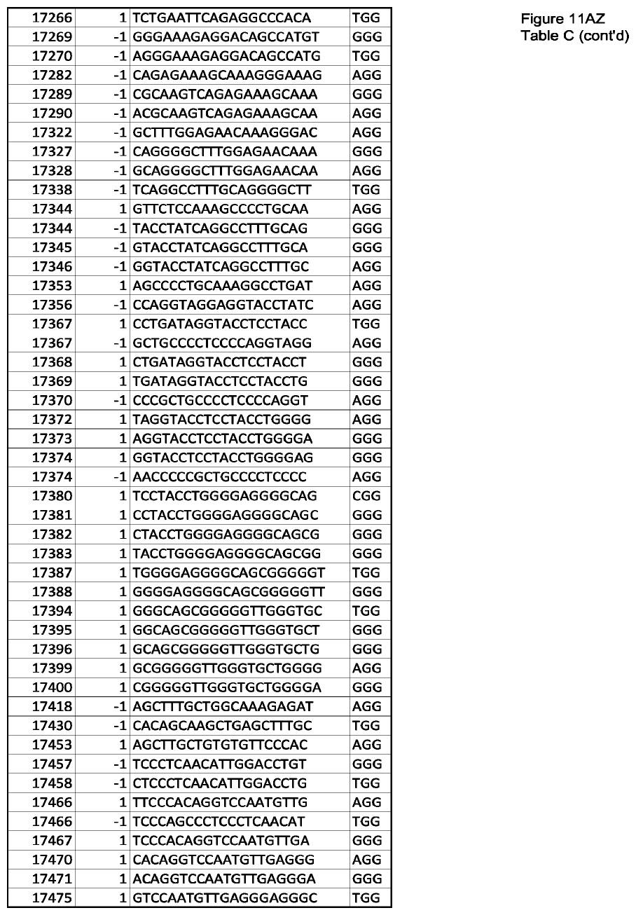

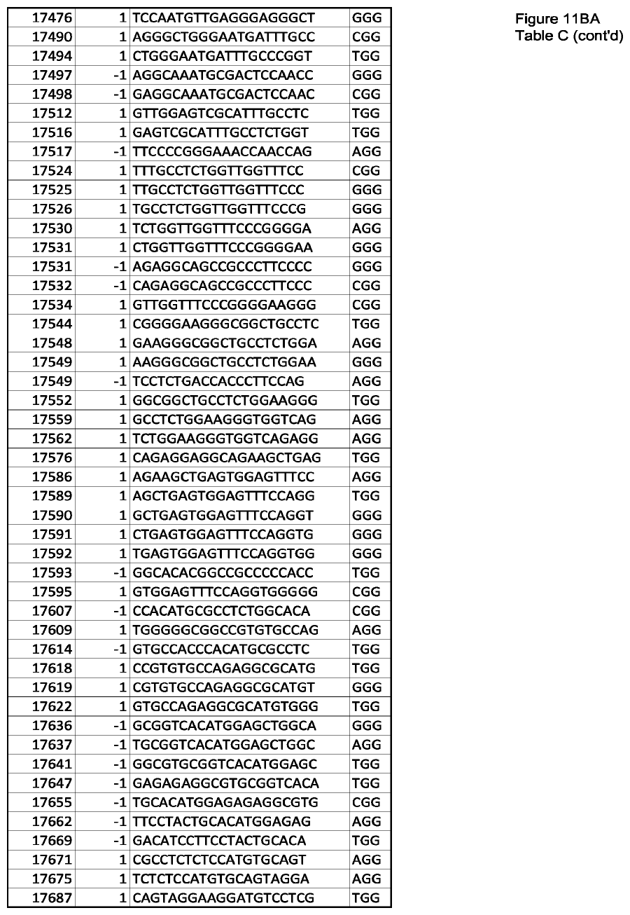

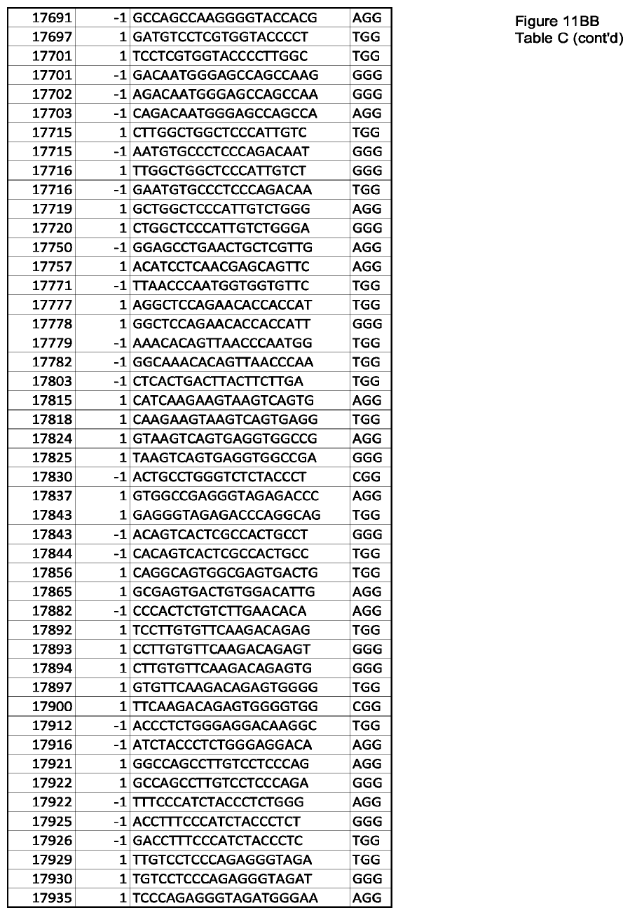

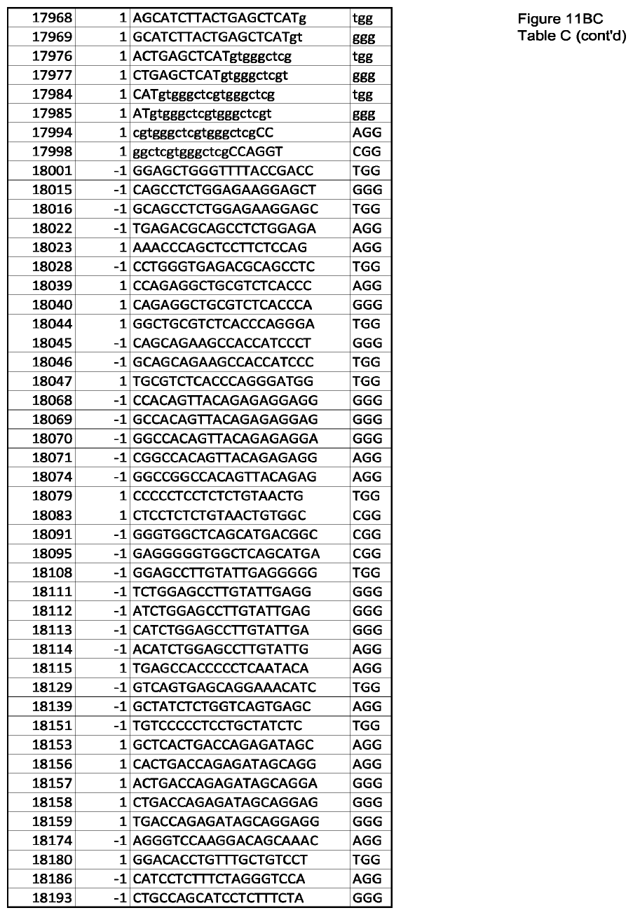

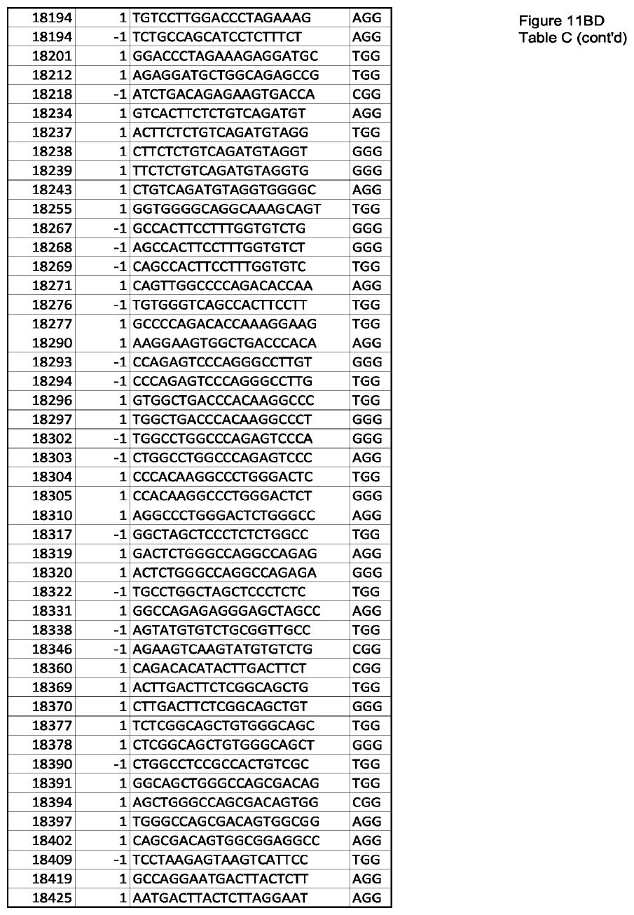

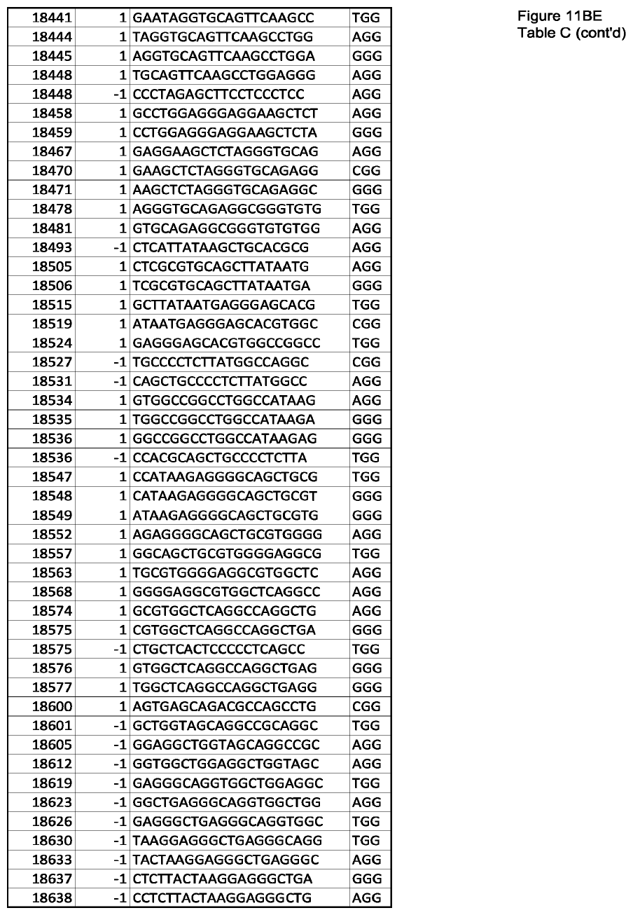

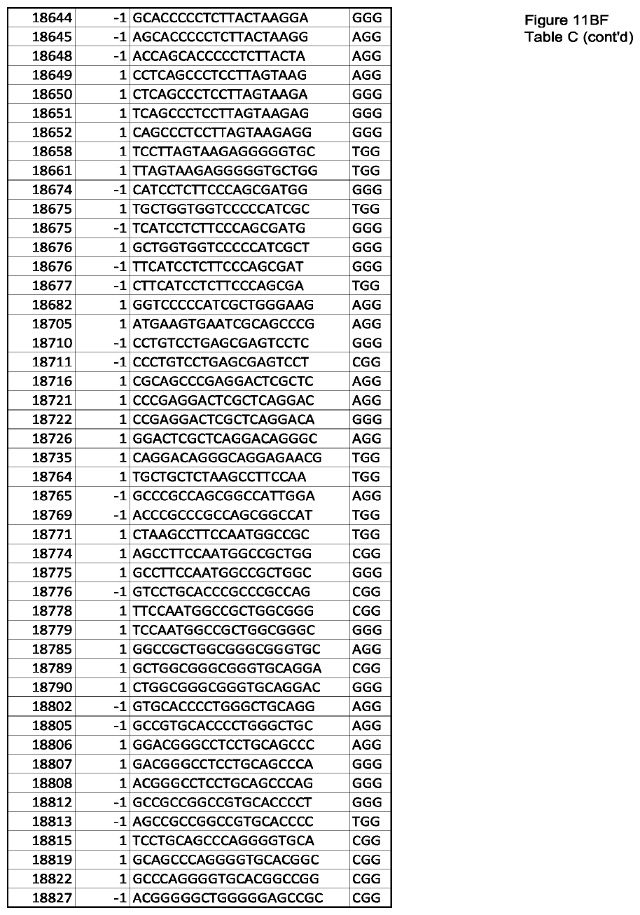

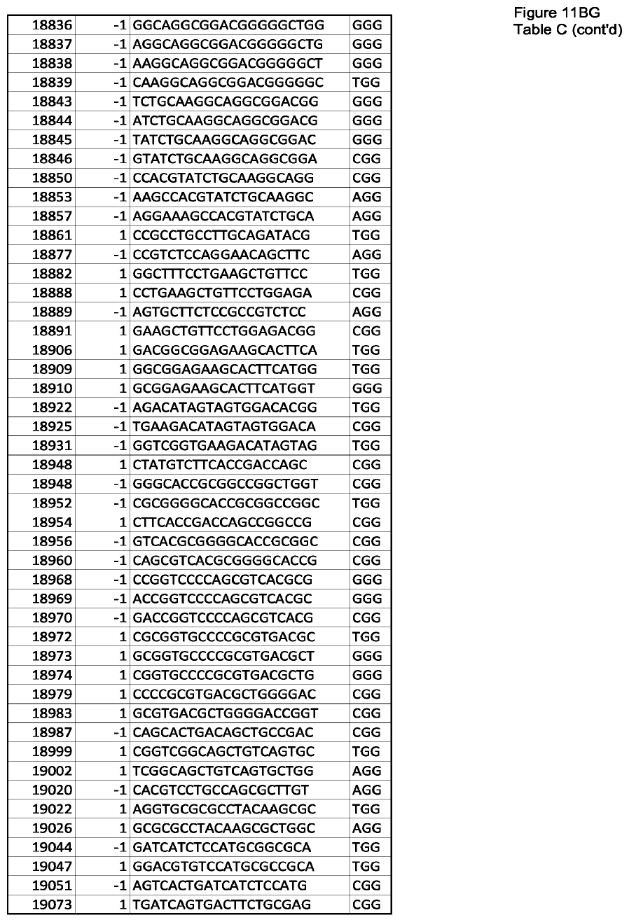

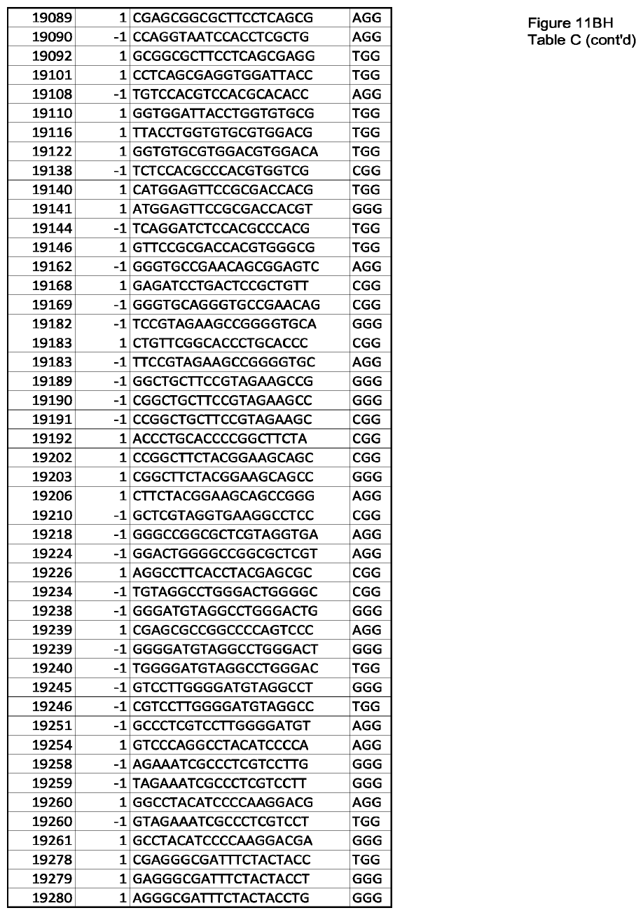

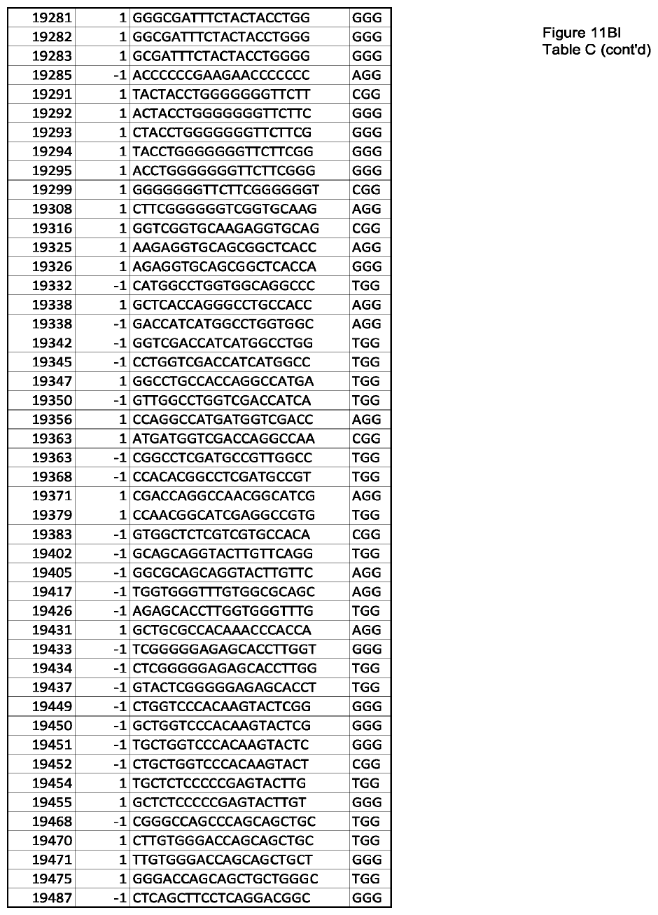

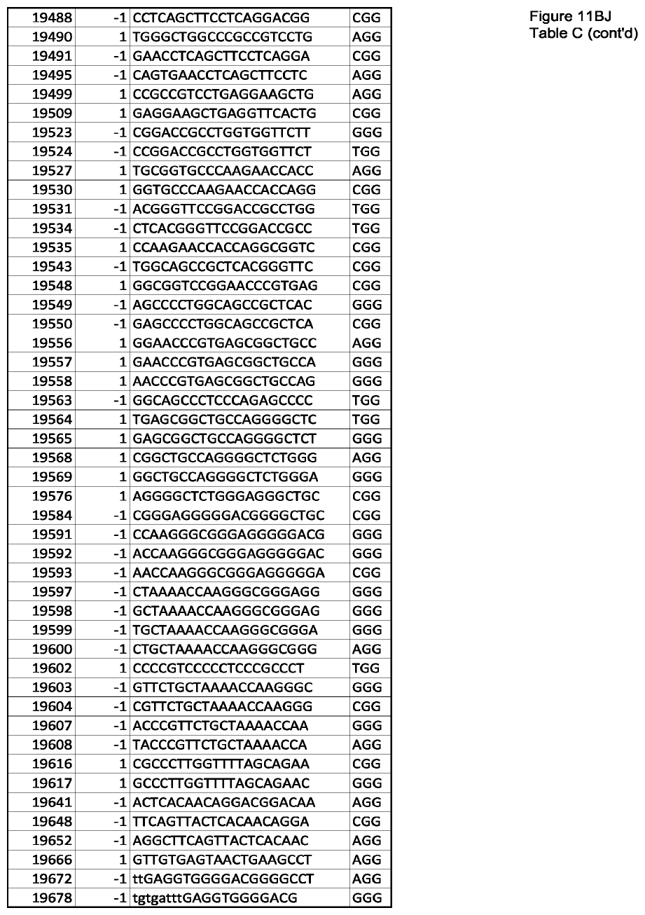

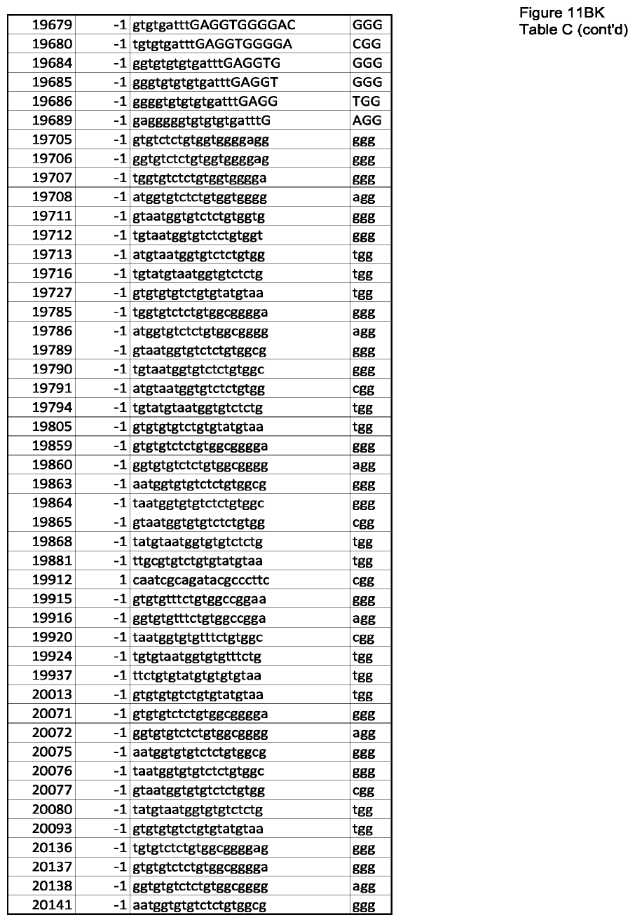

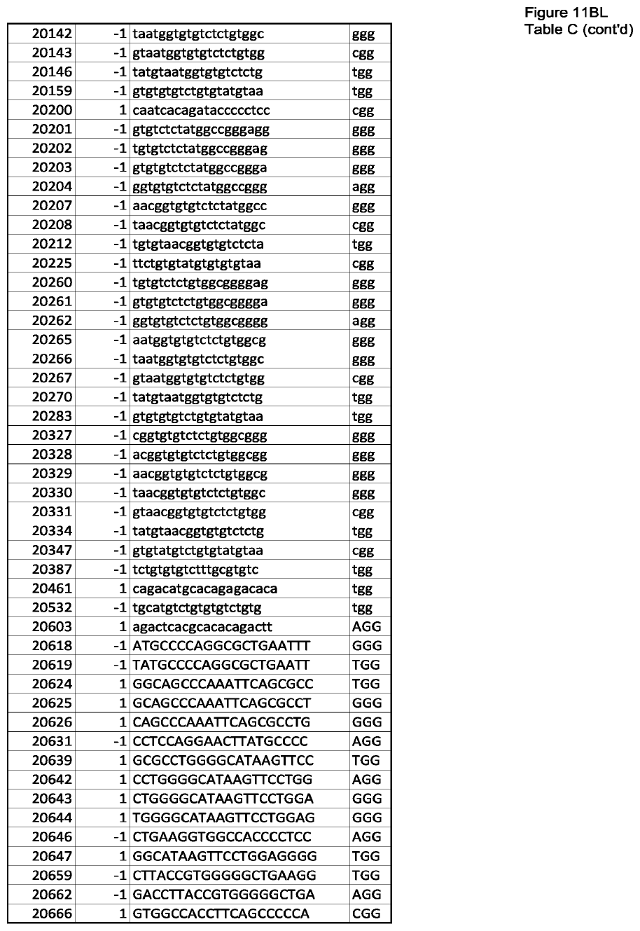

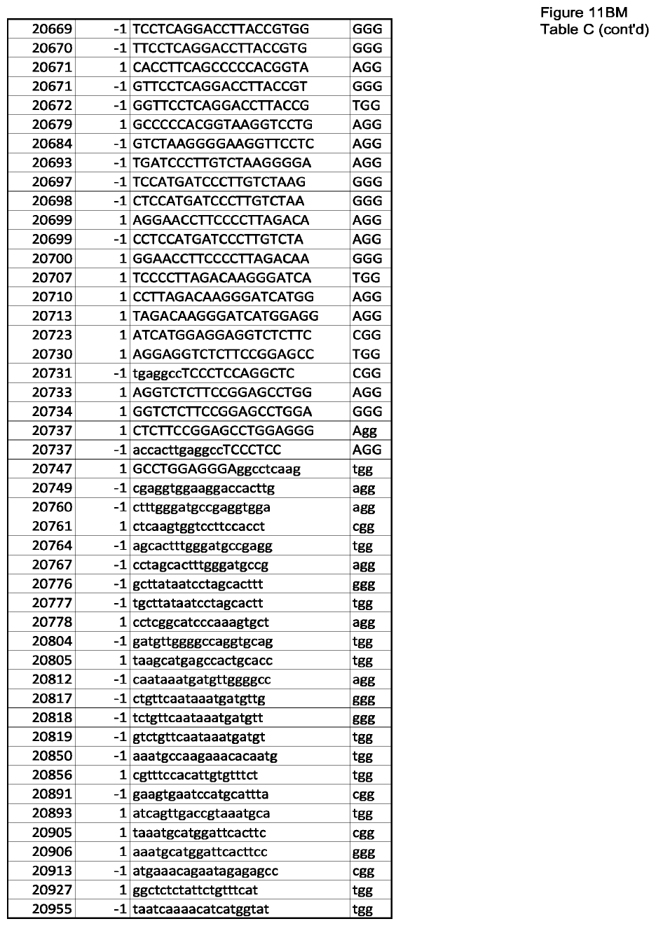

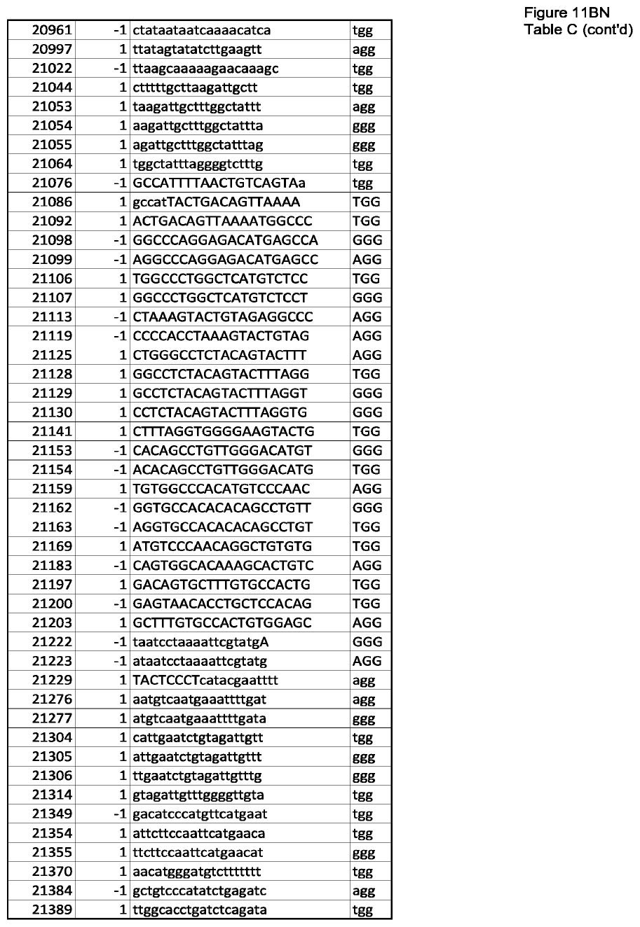

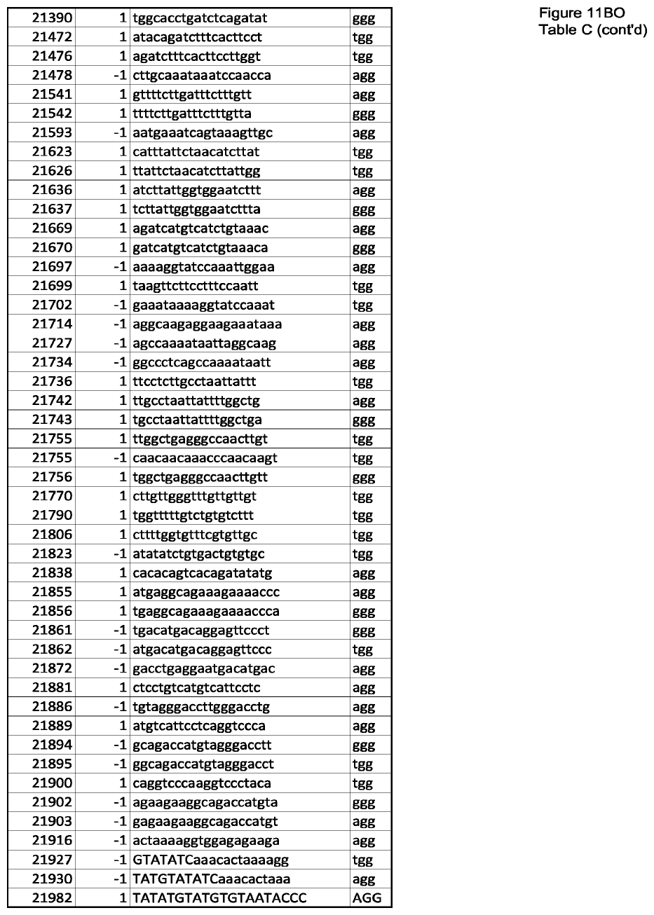

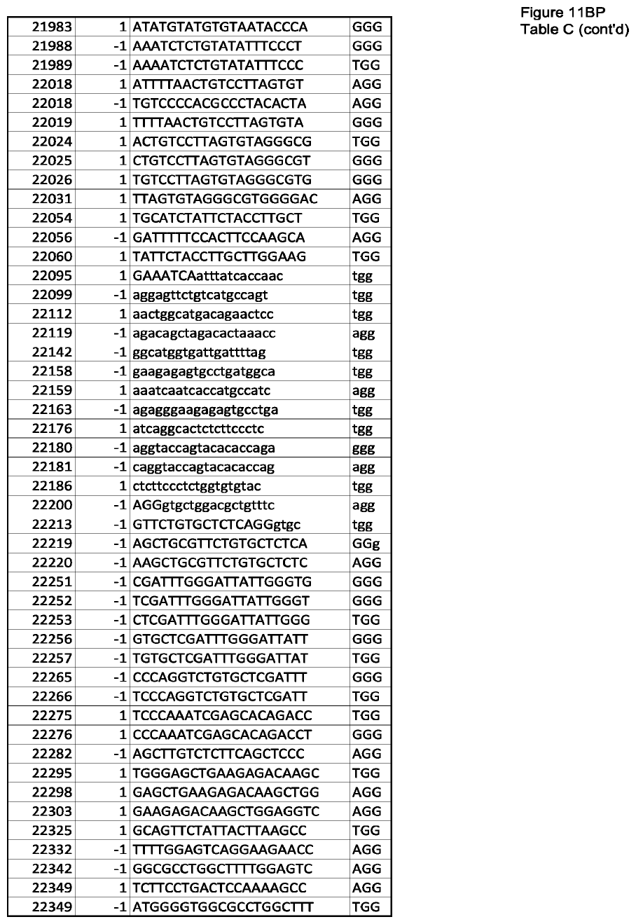

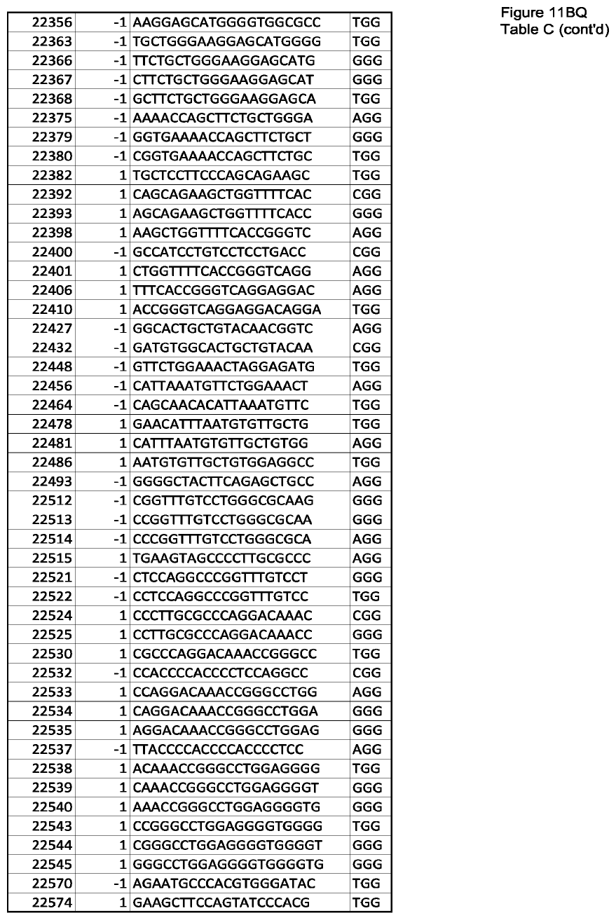

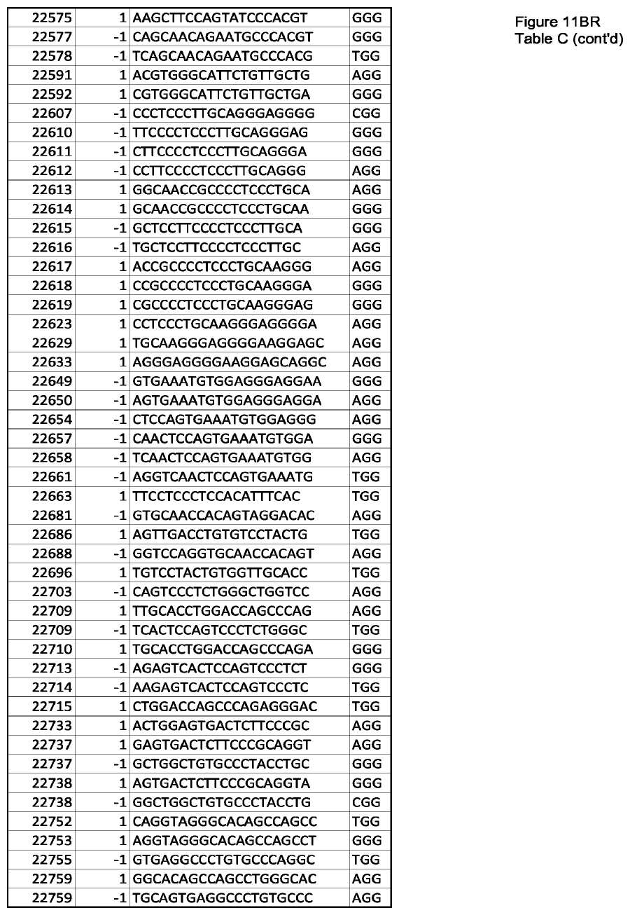

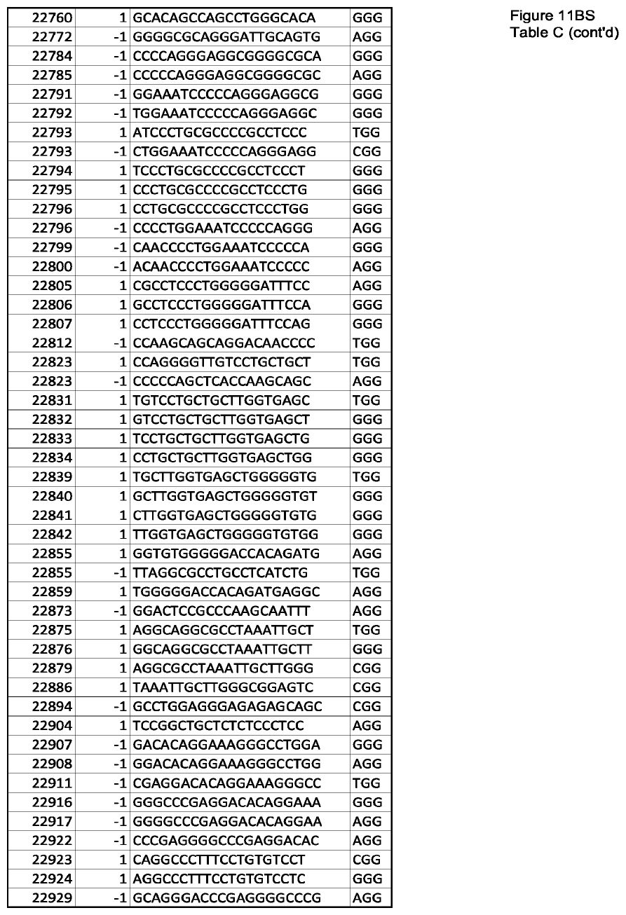

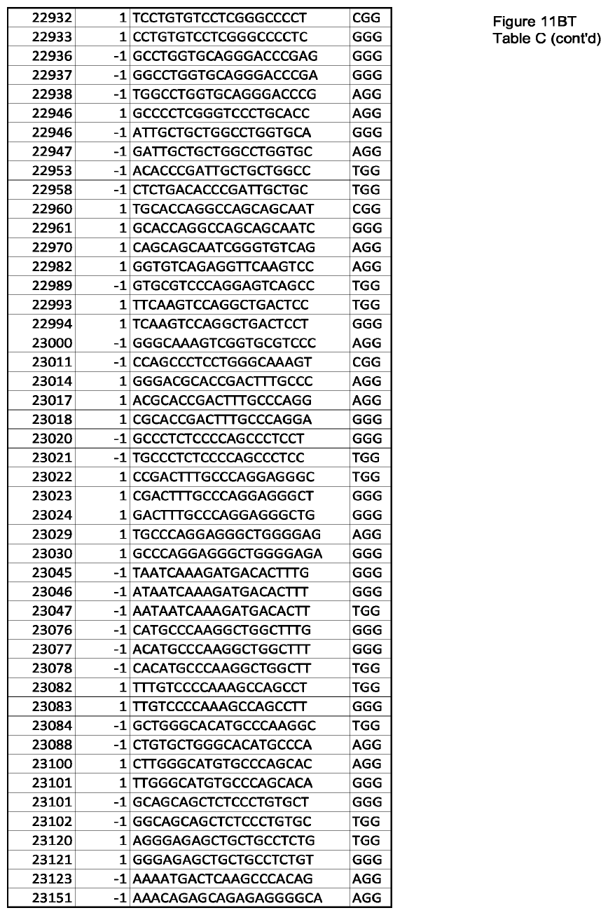

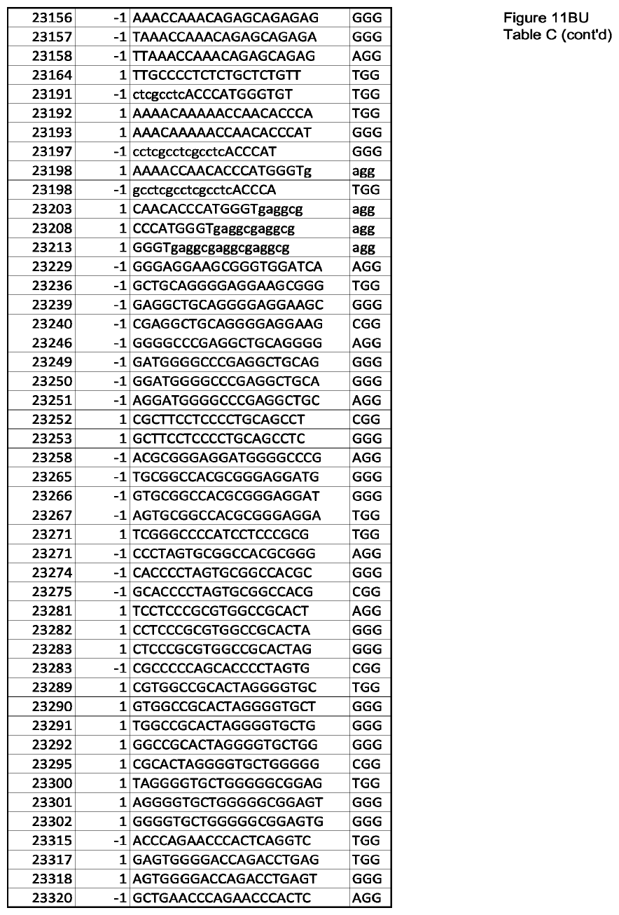

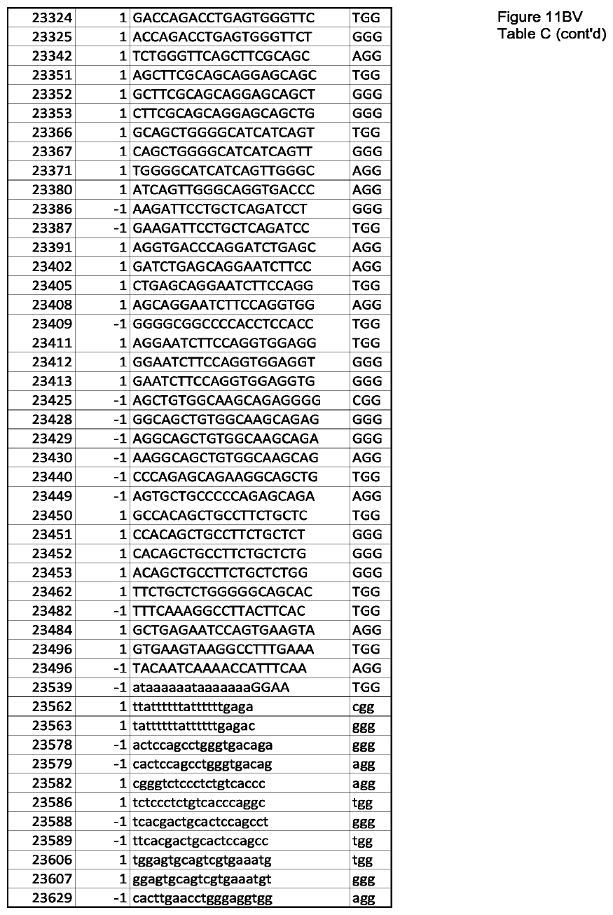

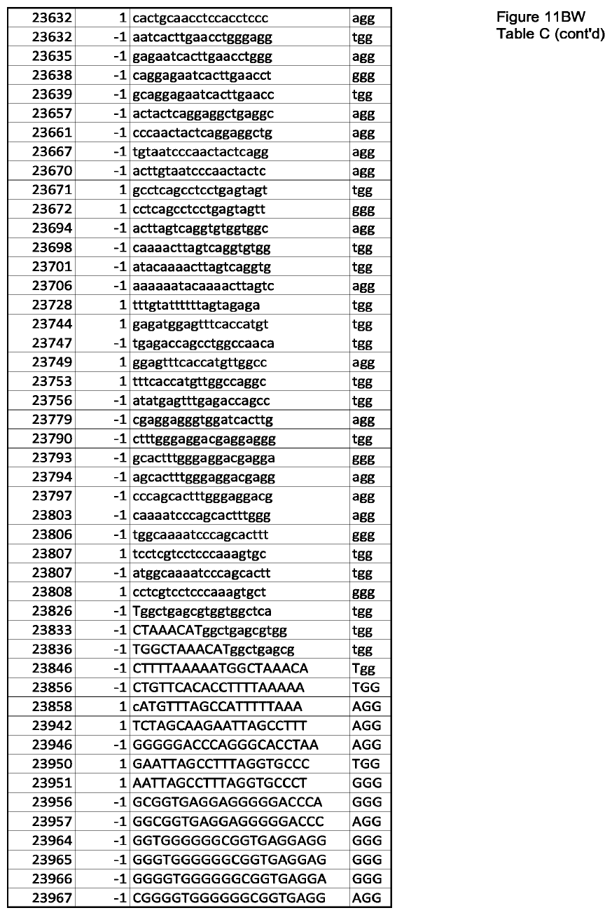

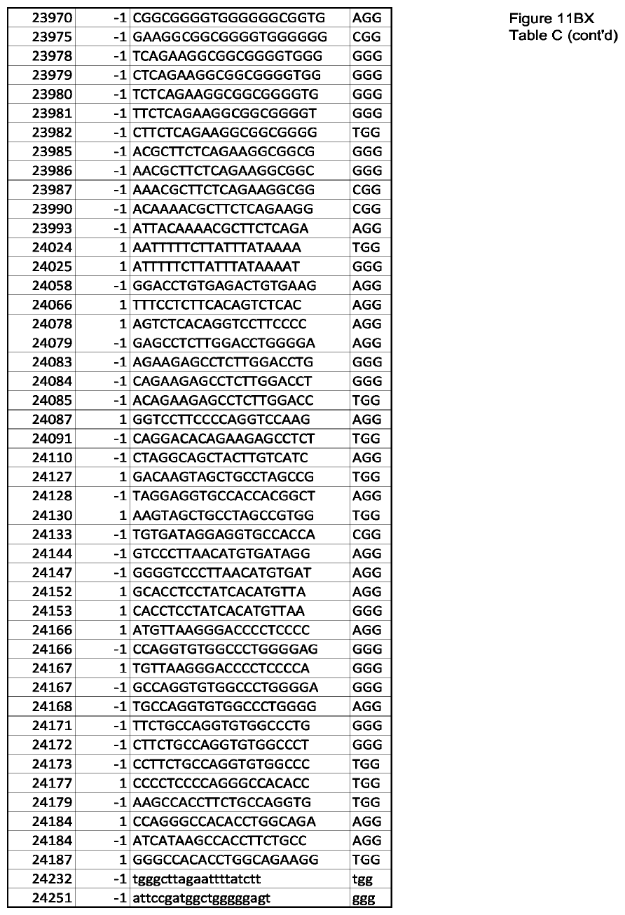

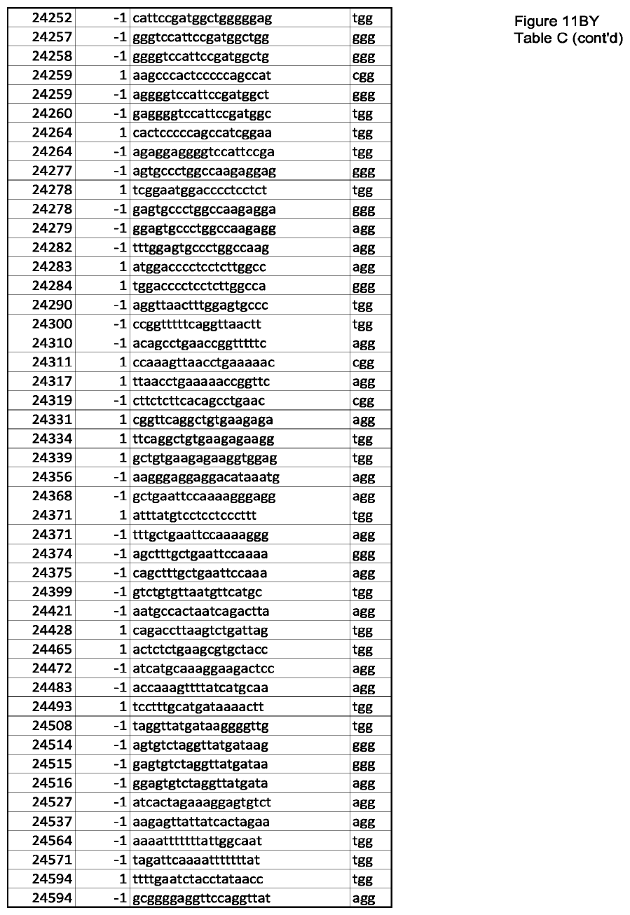

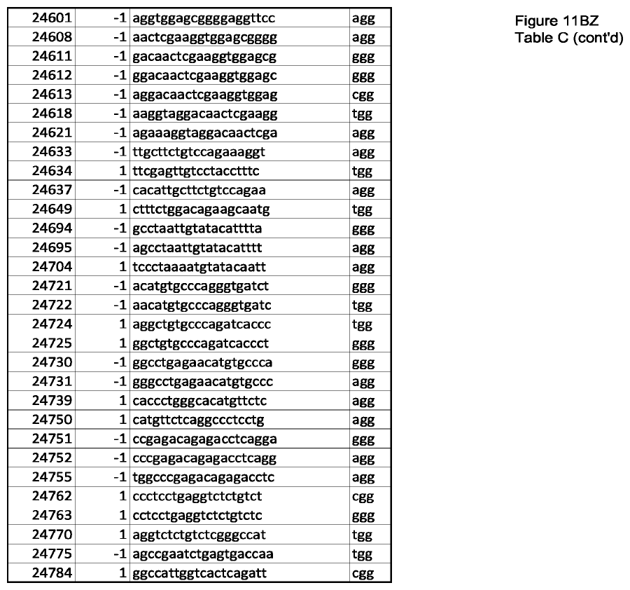

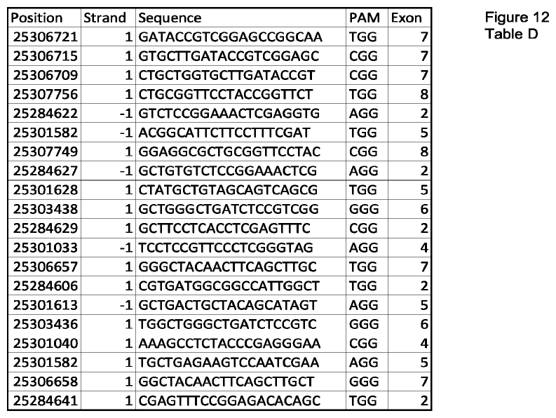

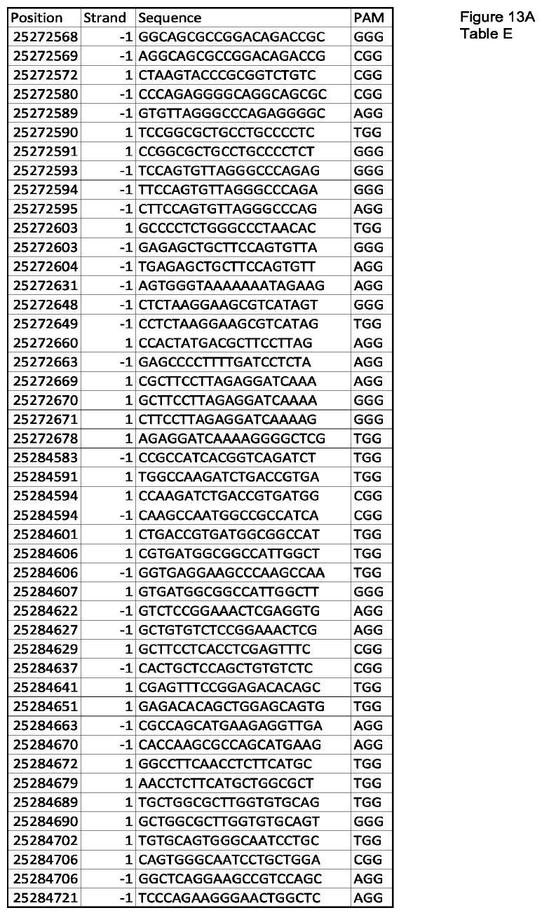

### DETAILED DESCRIPTION OF THE INVENTION

### I. Introduction

This invention is based, in part, on methods to genetically modify cells to produce a histo-blood antigen type suitable for universal transplantation. Described are methods of gene editing of ABO, RHD and FUT1 genes to produce universal histo-blood type cells. In some embodiments, cells harboring such gene modifications also carry gene alterations in genes regulating expression of MHC I and/or MHC II antigens. The cells outlined herein can improve and facilitate implementation and delivery of cellular therapies to patients.

### II. Definitions

As used herein, “immunogenicity” refers to property that allows a substance to induce a detectable immune response (humoral or cellular) when introduced into a subject (e.g., a human subject).

As used herein to characterize a cell, the term “hypoimmunogenic” generally means that such cell is less prone to immune rejection by a subject into which such cells are transplanted. For example, relative to an unaltered or unmodified wild-type cell, such a hypoimmunogenic cell may be about 2.5%, 5%, 10%, 20%, 30%, 40%, 50%, 60%, 70%, 80%, 90%, 95%, 97.5%, 99% or more less prone to immune rejection by a subject into which such cells are transplanted. In some aspects, genome editing technologies are used to modulate the expression of MHC I and MHC II genes, and thus, generate a hypoimmunogenic cell. In some embodiments, a hypoimmunogenic cell evades immune rejection in an MHC-mismatched allogenic recipient. In some instance, differentiated cells produced from the hypoimmunogenic stem cells outlined herein evade immune rejection when administered (e.g., transplanted or grafted) to an MHC-mismatched allogenic recipient. In some embodiments, a hypoimmunogenic cell is protected from T cell-mediated adaptive immune rejection and/or innate immune cell rejection.

Hypoimmunogencity of a cell can be determined by evaluating the immunogenicity of the cell such as the cell's ability to elicit adaptive and innate immune responses. Such immune response can be measured using assays recognized by those skilled in the art. In some embodiments, an immune response assay measures the effect of a hypoimmunogenic cell on T cell proliferation, T cell activation, T cell killing, NK cell proliferation, NK cell activation, and macrophage activity. In some cases, hypoimmunogenic cells and derivatives thereof undergo decreased killing by T cells and/or NK cells upon administration to a subject. In some instances, the cells and derivatives thereof show decreased macrophage engulfment compared to an unmodified or wildtype cell. In some embodiments, a hypoimmunogenic cell elicits a reduced or diminished immune response in a recipient subject compared to a corresponding unmodified wild-type cell. In some embodiments, a hypoimmunogenic cell is nonimmunogenic or fails to elicit an immune response in a recipient subject.

By “HLA” or “human leukocyte antigen” complex is a gene complex encoding the major histocompatibility complex (MHC) proteins in humans. These cell-surface proteins that make up the HLA complex are responsible for the regulation of the immune response to antigens. In humans, there are two MHCs, class I and class II, “HLA-I” and “HLA-II”. HLA-I includes three proteins, HLA-A, HLA-B and HLA-C, which present peptides from the inside of the cell, and antigens presented by the HLA-I complex attract killer T cells (also known as CD8+ T cells or cytotoxic T cells). The HLA-I proteins are associated with β-2 microglobulin (B2M). HLA-II includes five proteins, HLA-DP, HLA-DM, HLA-DOB, HLA-DQ and HLA-DR, which present antigens from outside the cell to T lymphocytes. This stimulates CD4+ cells (also known as T-helper cells). It should be understood that the use of either “MHC” or “HLA” is not meant to be limiting, as it depends on whether the genes are from humans (HLA) or murine (MHC). Thus, as it relates to mammalian cells, these terms may be used interchangeably herein.

As used herein, the terms “evade rejection,” “escape rejection,” “avoid rejection,” and similar terms are used interchangeably to refer to genetically or otherwise modified membranous products and cells according to the invention that are less susceptible to rejection when transplanted into a subject when compared with corresponding products and cells that are not genetically modified according to the invention. In some embodiments, the genetically modified products and cells according to the invention are less susceptible to rejection when transplanted into a subject when compared with corresponding cells that are ABO blood group or Rh factor mismatched to the subject.

By “allogeneic” herein is meant the genetic dissimilarity of a host organism and a cellular transplant where an immune cell response is generated.

The term “pluripotent cells” refers to cells that can self-renew and proliferate while remaining in an undifferentiated state and that can, under the proper conditions, be induced to differentiate into specialized cell types. The term “pluripotent cells,” as used herein, encompass embryonic stem cells and other types of stem cells, including fetal, amnionic, or somatic stem cells. Exemplary human stem cell lines include the H9 human embryonic stem cell line, RUES2 human embryonic stem cell line, and the like. Additional exemplary stem cell lines include those made available through the National Institutes of Health Human Embryonic Stem Cell Registry and the Howard Hughes Medical Institute HUES collection, (as described in Cowan, C. A. et. al, New England J. Med. 350:13. (2004), incorporated by reference herein in its entirety. “Pluripotent stem cells” as used herein have the potential to differentiate into any of the three germ layers: endoderm (e.g., the stomach linking, gastrointestinal tract, lungs, etc.), mesoderm (e.g., muscle, bone, blood, urogenital tissue, etc.) or ectoderm (e.g. epidermal tissues and nervous system tissues). The term “pluripotent stem cells,” as used herein, also encompasses “induced pluripotent stem cells”, or “iPSCs”, a type of pluripotent stem cell derived from a non-pluripotent cell. Examples of parent cells include somatic cells that have been reprogrammed to induce a pluripotent, undifferentiated phenotype by various means. Such “iPS” or “iPSC” cells can be created by inducing the expression of certain regulatory genes or by the exogenous application of certain proteins. Methods for the induction of iPS cells are known in the art and are further described below. (See, e.g., Zhou et al., Stem Cells 27 (11): 2667-74 (2009); Huangfu et al, Nature Biotechnol. 26 (7): 795 (2008); Woltjen et al., Nature 458 (7239): 766-770 (2009); and Zhou et al., Cell Stem Cell 8:381-384 (2009); each of which is incorporated by reference herein in their entirety.) The generation of induced pluripotent stem cells (iPSCs) is outlined below. As used herein, “hiPSCs” are human induced pluripotent stem cells.

Several characteristics of pluripotent stem cells distinguish them from other cells. For example, the ability to give rise to progeny that can undergo differentiation, under the appropriate conditions, into cell types that collectively demonstrate characteristics associated with cell lineages from all of the three germinal layers (e.g., endoderm, mesoderm, and ectoderm) is a pluripotent stem cell characteristic. Expression or non-expression of certain combinations of molecular markers are also pluripotent stem cell characteristics. For example, human pluripotent stem cells express at least several, and in some embodiments, all of the markers from the following non-limiting list: SSEA-3, SSEA-4, TRA-1-60, TRA-1-81, TRA-2-49/6E, ALP, Sox2, E-cadherin, UTF-1, Oct4, Rex1, and Nanog. Cell morphologies associated with pluripotent stem cells are also pluripotent stem cell characteristics. Cells do not need to pass through pluripotency to be reprogrammed into endodermal progenitor cells and/or hepatocytes.

As used herein, “non-pluripotent cells” refer to mammalian cells that are not pluripotent cells. Examples of such cells include differentiated cells as well as progenitor cells. Examples of differentiated cells include, but are not limited to, cells from a tissue selected from bone marrow, skin, skeletal muscle, fat tissue and peripheral blood. Exemplary cell types include, but are not limited to, fibroblasts, hepatocytes, myoblasts, neurons, osteoblasts, osteoclasts, and T cells. The starting cells employed for generating the induced multipotent cells, the endodermal progenitor cells, and the hepatocytes can be non-pluripotent cells.

As used herein, “multipotent” or “multipotent cell” refers to a cell type that can give rise to a limited number of other particular cell types. For example, induced multipotent cells are capable of forming endodermal cells. Additionally, multipotent blood stem cells can differentiate itself into several types of blood cells, including lymphocytes, monocytes, neutrophils, etc.

Differentiated cells include, but are not limited to, multipotent cells, oligopotent cells, unipotent cells, progenitor cells, and terminally differentiated cells. In particular embodiments, a less potent cell is considered “differentiated” in reference to a more potent cell.

A “somatic cell” is a cell forming the body of an organism. Somatic cells include cells making up organs, skin, blood, bones and connective tissue in an organism, but not germ cells. Somatic cell as used herein includes but is not limited to cardiac cells, liver cell, kidney cells, pancreatic cells, neural cells, glial cells, immune cells, mesenchymal cells, epithelial cells, and endothelial cells.

In some embodiments, the term “immune cell” as used herein includes immune effector cells, e.g., as described herein. Exemplary immune effector cells include T cells, e.g., alpha/beta T cells and gamma/delta T cells, B cells, natural killer (NK) cells, natural killer T (NKT) cells, mast cells, myeloid-derived phagocytes, or a combination thereof.

By “hypoimmunogenic pluripotent cell,” “hypoimmune pluripotent cell” or “HIP cell” herein is meant a pluripotent cell that retains its pluripotent characteristics and yet gives rise to a reduced immunological rejection response when transferred into an allogeneic host. In preferred embodiments, HIP cells do not give rise to an immune response. Thus, “hypo-immunogenic” refers to a significantly reduced or eliminated immune response when compared to the immune response of a parental (i.e. “wt”) cell prior to immunoengineering as outlined herein. In many cases, the HIP cells are immunologically silent and yet retain pluripotent capabilities. Assays for HIP characteristics are outlined below.

By “wild type” or “wt” in the context of a cell means a cell found in nature. However, in the context of a pluripotent stem cell, as used herein, it also means an iPSC that may contain nucleic acid changes resulting in pluripotency but did not undergo the gene editing procedures of the invention to achieve hypo-immunogenicity.

The terms “treat”, “treating”, “treatment”, etc., as applied to an isolated cell, include subjecting the cell to any kind of process or condition or performing any kind of manipulation or procedure on the cell. As applied to a subject, the terms refer to administering a cell or population of cells in which a target polynucleotide sequence (e.g., B2M) has been altered ex vivo according to the methods described herein to an individual. The individual is usually ill or injured, or at increased risk of becoming ill relative to an average member of the population and in need of such attention, care, or management.

As used herein, the term “treating” and “treatment” refers to administering to a subject an effective amount of cells with target polynucleotide sequences altered ex vivo according to the methods described herein so that the subject has a reduction in at least one symptom of the disease or an improvement in the disease, for example, beneficial or desired clinical results. For purposes of this invention, beneficial or desired clinical results include, but are not limited to, alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable. Treating can refer to prolonging survival as compared to expected survival if not receiving treatment. Thus, one of skill in the art realizes that a treatment may improve the disease condition, but may not be a complete cure for the disease. As used herein, the term “treatment” includes prophylaxis. Alternatively, treatment is “effective” if the progression of a disease is reduced or halted. “Treatment” can also mean prolonging survival as compared to expected survival if not receiving treatment. Those in need of treatment include those already diagnosed with a disorder associated with expression of a polynucleotide sequence, as well as those likely to develop such a disorder due to genetic susceptibility or other factors.

By “treatment” or “prevention” of a disease or disorder is meant delaying or preventing the onset of such a disease or disorder, reversing, alleviating, ameliorating, inhibiting, slowing down or stopping the progression, aggravation or deterioration the progression or severity of a condition associated with such a disease or disorder. In one embodiment, the symptoms of a disease or disorder are alleviated by at least 5%, at least 10%, at least 20%, at least 30%, at least 40%, or at least 50%.

As used herein, the terms “administering,” “introducing” and “transplanting” are used interchangeably in the context of the placement of cells, e.g. cells described herein comprising a target polynucleotide sequence altered according to the methods of the invention into a subject, by a method or route which results in at least partial localization of the introduced cells at a desired site. The cells can be implanted directly to the desired site, or alternatively be administered by any appropriate route which results in delivery to a desired location in the subject where at least a portion of the implanted cells or components of the cells remain viable. The period of viability of the cells after administration to a subject can be as short as a few hours, e. g. twenty-four hours, to a few days, to as long as several years. In some instances, the cells can also be administered a location other than the desired site, such as in the liver or subcutaneously, for example, in a capsule to maintain the implanted cells at the implant location and avoid migration of the implanted cells.

The term “effective amount” as used herein means an amount of a pharmaceutical composition which is sufficient enough to significantly and positively modify the symptoms and/or conditions to be treated (e.g., provide a positive clinical response). The effective amount of an active ingredient for use in a pharmaceutical composition will vary with the particular condition being treated, the severity of the condition, the duration of treatment, the nature of concurrent therapy, the particular active ingredient(s) being employed, the particular pharmaceutically-acceptable excipient(s) and/or carrier(s) utilized, and like factors with the knowledge and expertise of the attending physician.

The term “pharmaceutically acceptable” as used herein, refers to excipients, compositions and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The RNA molecule that binds to CRISPR-Cas components and targets them to a specific location within the target DNA is referred to herein as “guide RNA,” “gRNA,” or “small guide RNA” and may also be referred to herein as a “DNA-targeting RNA.” A guide RNA comprises at least two nucleotide segments: at least one “DNA-binding segment” and at least one “polypeptide-binding segment.” By “segment” is meant a part, section, or region of a molecule, e.g., a contiguous stretch of nucleotides of an RNA molecule. The definition of “segment,” unless otherwise specifically defined, is not limited to a specific number of total base pairs. In some embodiments, the targeting is accomplished through hybridization of a portion of the gRNA to DNA (e.g., through the gRNA targeting domain), and by binding of a portion of the gRNA molecule to the RNA-guided nuclease or other effector molecule (e.g., through at least the gRNA tracr). In some embodiments, a gRNA molecule consists of a single contiguous polynucleotide molecule, referred to herein as a “single guide RNA” or “sgRNA” and the like. In other embodiments, a gRNA molecule includes a plurality, usually two, polynucleotide molecules, which are themselves capable of association, usually through hybridization, referred to herein as a “dual guide RNA” or “dgRNA,” and the like. gRNA molecules are described in more detail below, and generally include a targeting domain and a tracr. In other embodiments the targeting domain and tracr are disposed on a single polynucleotide. The guide RNA can be introduced into the target cell as an isolated RNA molecule, or is introduced into the cell using an expression vector containing DNA encoding the guide RNA.

The term “guide RNA target” as used herein includes an RNA sequence of each and any of the guide RNA targets described herein and variants thereof which are utilized for gene editing. In some embodiment, the guide RNA target includes a target sequence to which a guide RNA binds, thereby allowing for gene editing of the target sequence. The guide RNA target can correspond to a target sequence and does not include a PAM sequence.

The “DNA-binding segment” (or “DNA-targeting sequence”) of the guide RNA comprises a nucleotide sequence that is complementary to a specific sequence within a target DNA.

The guide RNA can include one or more polypeptide-binding sequences/segments. The polypeptide-binding segment (or “protein-binding sequence”) of the guide RNA interacts with the RNA-binding domain of a Cas protein.

The term “Cas9 molecule,” as used herein, refers to Cas9 wild-type proteins derived from Type II CRISPR-Cas9 systems, modifications of Cas9 proteins, variants of Cas9 proteins, Cas9 orthologs, and combinations thereof.

The term “donor polynucleotide,” “donor template” and “donor oligonucleotide” are used interchangeably and refer to a polynucleotide that provides a nucleic acid sequence of which at least a portion is intended to be integrated into a selected nucleic acid target site. Generally speaking, a donor polynucleotide is a single-strand polynucleotide or a double-strand polynucleotide. For example, an engineered Type II CRISPR-Cas9 system can be used in combination with a donor DNA template to modify a DNA target sequence in a genomic DNA wherein the genomic DNA is modified to comprise at least a portion of the donor DNA template at the DNA target sequence. In some embodiments, a vector comprises a donor polynucleotide In other embodiments, a donor polynucleotide is an oligonucleotide.

The term “HDR”, as used herein, refers to homology-directed repair, as used herein, refers to the process of repairing DNA damage using a homologous nucleic acid (e.g., an endogenous homologous sequence, e.g., a sister chromatid, or an exogenous nucleic acid, e.g., a template nucleic acid). HDR typically acts when there has been significant resection at the double strand break, forming at least one single stranded portion of DNA. In a normal cell, HDR typically involves a series of steps such as recognition of the break, stabilization of the break, resection, stabilization of single stranded DNA, formation of a DNA crossover intermediate, resolution of the crossover intermediate, and ligation. In some cases, HDR requires nucleotide sequence homology and uses a donor template (e.g., a donor DNA template) or donor oligonucleotide to repair the sequence wherein the double-strand break occurred (e.g., DNA target sequence). This results in the transfer of genetic information from, for example, the donor template DNA to the DNA target sequence. HDR may result in alteration of the DNA target sequence (e.g., insertion, deletion, mutation) if the donor template DNA sequence or oligonucleotide sequence differs from the DNA target sequence and part or all of the donor template DNA polynucleotide or oligonucleotide is incorporated into the DNA target sequence. In some embodiments, an entire donor template DNA polynucleotide, a portion of the donor template DNA polynucleotide, or a copy of the donor polynucleotide is integrated at the site of the DNA target sequence.

The term “non-homologous end joining” or “NHEJ”, as used herein, refers to ligation mediated repair and/or non-template mediated repair.

The methods of the present invention can be used to alter a target polynucleotide sequence in a cell. The present invention contemplates altering target polynucleotide sequences in a cell for any purpose. In some embodiments, the target polynucleotide sequence in a cell is altered to produce a mutant cell. As used herein, a “mutant cell” refers to a cell with a resulting genotype that differs from its original genotype. In some instances, a “mutant cell” exhibits a mutant phenotype, for example when a normally functioning gene is altered using the CRISPR/Cas systems of the present invention. In other instances, a “mutant cell” exhibits a wild-type phenotype, for example when a CRISPR/Cas system of the present invention is used to correct a mutant genotype. In some embodiments, the target polynucleotide sequence in a cell is altered to correct or repair a genetic mutation (e.g., to restore a normal phenotype to the cell). In some embodiments, the target polynucleotide sequence in a cell is altered to induce a genetic mutation (e.g., to disrupt the function of a gene or genomic element).

In some embodiments, the alteration is an indel. As used herein, “indel” refers to a mutation resulting from an insertion, deletion, or a combination thereof. As will be appreciated by those skilled in the art, an indel in a coding region of a genomic sequence will result in a frameshift mutation, unless the length of the indel is a multiple of three. In some embodiments, the alteration is a point mutation. As used herein, “point mutation” refers to a substitution that replaces one of the nucleotides. A CRISPR/Cas system of the present invention can be used to induce an indel of any length or a point mutation in a target polynucleotide sequence.

As used herein, “knock out” includes deleting all or a portion of the target polynucleotide sequence in a way that interferes with the function of the target polynucleotide sequence. For example, a knock out can be achieved by altering a target polynucleotide sequence by inducing an indel in the target polynucleotide sequence in a functional domain of the target polynucleotide sequence (e.g., a DNA binding domain). Those skilled in the art will readily appreciate how to use the CRISPR/Cas systems of the present invention to knock out a target polynucleotide sequence or a portion thereof based upon the details described herein.

The process of a “gene knock-out” renders a particular gene inactive in the host cell in which it resides, resulting either in no protein of interest being produced or an inactive form. As will be appreciated by those in the art and further described below, this can be accomplished in a number of different ways, including removing nucleic acid sequences from a gene, or interrupting the sequence with other sequences, altering the reading frame, or altering the regulatory components of the nucleic acid. For example, all or part of a coding region of the gene of interest can be removed or replaced with “nonsense” sequences, all or part of a regulatory sequence such as a promoter can be removed or replaced, translation initiation sequences can be removed or replaced, etc.

In some embodiments, the alteration results in a knock out of the target polynucleotide sequence or a portion thereof. Knocking out a target polynucleotide sequence or a portion thereof using a CRISPR/Cas system of the present invention can be useful for a variety of applications. For example, knocking out a target polynucleotide sequence in a cell can be performed in vitro for research purposes. For ex vivo purposes, knocking out a target polynucleotide sequence in a cell can be useful for treating or preventing a disorder associated with expression of the target polynucleotide sequence (e.g., by knocking out a mutant allele in a cell ex vivo and introducing those cells comprising the knocked out mutant allele into a subject).

By “knock in” herein is meant a process that adds a genetic function to a host cell. This causes increased levels of the encoded protein. As will be appreciated by those in the art, this can be accomplished in several ways, including adding one or more additional copies of the gene to the host cell or altering a regulatory component of the endogenous gene increasing expression of the protein is made. This may be accomplished by modifying the promoter, adding a different promoter, adding an enhancer, or modifying other gene expression sequences.

As used herein, a “vector” or “vectors” refer to tools that allow or facilitate the transfer of an entity from one environment to another.

The term “packaging cell” as used herein includes a cell which contains some or all of the elements necessary for packaging an infectious recombinant virus or viral vector. The packaging cell may lack a recombinant viral vector. Typically, such packaging cells contain one or more vectors which are capable of expressing viral structural proteins. Cells comprising only some of the elements required for the production of enveloped viral particles are useful as intermediate reagents in the generation of viral particle producer cell lines, through subsequent steps of transient transfection, transduction or stable integration of each additional required element. These intermediate reagents are encompassed by the term “packaging cell.”

As used herein, “viral particles” refer to replication-competent or -defective viruses, viral vectors derived therefrom, and which may or may not comprise a nucleotide of interest.

An “enveloped virus” refers to a virus containing a proteinaceous viral envelope which surrounds the viral capsid. Such enveloped viruses include orthomyxoviruses and paramyxovirus, herpes viruses, togaviruses, lentiviruses, and retroviruses. During cell infection by an enveloped virus, the plasma membrane of the host cell is altered to include some viral-coded proteins and, as the viral nucleoprotein core exits the host cell in which it was assembled, it becomes enveloped with the modified membrane, thus forming the viral envelope.

Lentivirus vectors are part of the larger group of retroviral vectors. A detailed list of lentiviruses may be found in Coffin, J. M. et al. (1997) Retroviruses, Cold Spring Harbor Laboratory Press, 758-63. Lentiviruses can be divided into primate and non-primate groups. Examples of primate lentiviruses include but are not limited to human immunodeficiency virus (HIV), the causative agent of human acquired immunodeficiency syndrome (AIDS); and simian immunodeficiency virus (SIV). Examples of non-primate lentiviruses include the prototype “slow virus” visna/maedi virus (VMV), as well as the related caprine arthritis-encephalitis virus (CAEV), equine infectious anaemia virus (EIAV), and the more recently described feline immunodeficiency virus (FIV) and bovine immunodeficiency virus (BIV).

The lentivirus family differs from retroviruses in that lentiviruses have the capability to infect both dividing and non-dividing cells (Lewis, P. et al. (1992) EMBO J 11: 3053-8; Lewis, P. F. et al. (1994) J. Virol. 68: 510-6). In contrast, other retroviruses, such as MLV, are unable to infect non-dividing or slowly dividing cells such as those that make up, for example, muscle, brain, lung and liver tissue.

The lentiviral vector may be a “primate” vector. The lentiviral vector may be a “non-primate” vector (i.e. derived from a virus which does not primarily infect primates, especially humans). Examples of non-primate lentiviruses may be any member of the family of lentiviridae which does not naturally infect a primate.

In some embodiments, the lentiviral vector is a pseudotyped lentiviral vector. Pseudotyped lentiviral vectors comprise vector particles bearing glycoproteins from other enveloped viruses. Pseudotyping is a mechanism for expanding the cellular tropism of enveloped viruses through the formation of phenotypically mixed particles or pseudotypes. In some embodiments, the vector may be pseudotyped with a fusogenic envelope G glycoprotein of the vesicular stomatitis virus (VSV-G). Pseudotyped proteins may also be derived from rabies virus, MLV, Ebola, baculovirus, paramyxoviruses (e.g., measles virus, Hendra virus, Nipah virus), and filovirus.

In some embodiments, the lentiviral vector is an HIV-1 or HIV-2 based vector. The HIV-1 vector contains cis-acting elements that are also found in simple retroviruses. It has been shown that sequences that extend into the gag open reading frame are important for packaging of HIV-1. Therefore, HIV-1 vectors often contain the relevant portion of gag in which the translational initiation codon has been mutated. In addition, most HIV-1 vectors also contain a portion of the env gene that includes the RRE. Rev binds to RRE, which permits the transport of full-length or singly spliced mRNAs from the nucleus to the cytoplasm. In the absence of Rev and/or RRE, full-length HIV-1 RNAs accumulate in the nucleus. Alternatively, a constitutive transport element from certain simple retroviruses such as Mason-Pfizer monkey virus can be used to relieve the requirement for Rev and RRE. Efficient transcription from the HIV-1 LTR promoter requires the viral protein Tat. Most HIV-2-based vectors are structurally very similar to HIV-1 vectors. Similar to HIV-1-based vectors, HIV-2 vectors also require RRE for efficient transport of the full-length or singly spliced viral RNAs. HIV-derived vectors for use in the present invention are not limited in terms of HIV strain.

In some embodiments, the plasmid vector used to produce the viral genome within a host cell/packaging cell will have sufficient lentiviral genetic information to allow packaging of an RNA genome, in the presence of packaging components, into a viral particle which is capable of infecting a target cell, but is incapable of independent replication to produce infectious viral particles within the final target cell. In a preferred embodiment, the vector lacks a functional gag-pol and/or env gene and/or other genes essential for replication. In some embodiments, the plasmid vector includes transcriptional regulatory control sequences operably linked to the lentiviral genome to direct transcription of the genome in a host cell/packaging cell. These regulatory sequences may be the natural sequences associated with the transcribed viral sequence (i.e. the 5′ U3 region), or they may be a heterologous promoter, such as another viral promoter (e.g. the CMV promoter).

In some embodiments, the vectors are integration-defective. Integration defective lentiviral vectors (IDLVs) can be produced, for example, either by packaging the vector with catalytically inactive integrase (such as an HIV integrase bearing the D64V mutation in the catalytic site; Naldini, L. et al. (1996) Science 272: 263-7; Naldini, L. et al. (1996) Proc. Natl. Acad. Sci. USA 93: 11382-8; Leavitt, A. D. et al. (1996) J. Virol. 70: 721-8) or by modifying or deleting essential att sequences from the vector LTR (Nightingale, S. J. et al. (2006) Mol. Ther. 13: 1121-32), or by a combination of the above.

In some embodiments, the lentiviral vector is a herpes simplex virus (HSV). Herpes simplex virus (HSV) is an enveloped double-stranded DNA virus that naturally infects neurons. HSV can accommodate large sections of foreign DNA, which makes it attractive as a vector system, and has been employed as a vector for gene delivery to neurons. The use of HSV in therapeutic procedures requires the strains to be attenuated so that they cannot establish a lytic cycle. If HSV vectors are to be used for gene therapy in humans, the gene of interest is preferably inserted into an essential gene. This is necessary, because if a vector virus encounters a wild type virus, transfer of a heterologous gene to the wild type virus could occur by recombination. However, as long as the NOI is inserted into an essential gene, recombinational transfer would also delete the essential gene in the recipient virus and prevent “escape” of the heterologous gene into the replication competent wild type virus population.

In other embodiments, the lentiviral vector is a vaccinia virus derived vector. Vaccinia virus is large enveloped virus that has an approximately 190 kb linear, double-stranded DNA genome. Vaccinia virus can accommodate up to approximately 25 kb of foreign DNA, which also makes it useful for the delivery of large genes. A number of attenuated vaccinia virus strains are known in the art that are suitable for gene therapy applications, for example the MVA and NYVAC strains.

A “nucleic acid,” “nucleic acid molecule,” “oligonucleotide” or “polynucleotide” means a polymeric compound comprising covalently linked nucleotides. The term “nucleic acid” includes polyribonucleic acid (RNA) and polydeoxyribonucleic acid (DNA), both of which may be single- or double-stranded. DNA includes, but is not limited to, complimentary DNA (cDNA), genomic DNA, plasmid or vector DNA, and synthetic DNA.

A “gene” refers to an assembly of nucleotides that encode a polypeptide, and includes cDNA and genomic DNA nucleic acid molecules. “Gene” also refers to a nucleic acid fragment that can act as a regulatory sequences preceding (5′ non-coding sequences) and following (3′ non-coding sequences) the coding sequence.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

An “amino acid” as used herein refers to a compound containing both a carboxyl (—COON) and amino (—NH.sub.2) group. “Amino acid” refers to both natural and unnatural, i.e., synthetic, amino acids. Natural amino acids, with their three-letter and single letter abbreviations, include Alanine (Ala; A); Arginine (Arg, R); Asparagine (Asn; N); Aspartic acid (Asp; D); Cysteine (Cys; C); Glutamine (Gln; Q); Glutamic acid (Glu; E); Glycine (Gly; G); Histidine (His; H); Isoleucine (Ile; I); Leucine (Leu; L); Lysine (Lys; K); Methionine (Met; M); Phenylalanine (Phe; F); Proline (Pro; P); Serine (Ser; S); Threonine (Thr; T); Tryptophan (Trp; W); Tyrosine (Tyr; Y); and Valine (Val; V).

An “amino acid substitution” refers to a polypeptide or protein comprising one or more substitutions of a wild-type or naturally occurring amino acid with a different amino acid relative to the wild-type or naturally occurring amino acid at that amino acid residue. The substituted amino acid of the invention may be a synthetic or naturally occurring amino acid. In certain embodiments, the substituted amino acid is a naturally occurring amino acid selected from the group consisting of: A, R, N, D, C, Q, E, G, H, I, L, K, M, F, P, S, T, W, Y, and V. Substitution mutants may be described using an abbreviated system. For example, a substitution mutation in which the fifth (5.sup.th) amino acid residue is substituted may be abbreviated as “X5Y,” wherein “X” is the wild-type or naturally occurring amino acid to be replaced, “5” is the amino acid residue within the protein or polypeptide, and “Y” is the substituted, or non-wild-type or non-naturally occurring, amino acid.

The term “recombinant” when used in reference to a nucleic acid molecule, peptide, polypeptide, or protein means of, or resulting from, a new combination of genetic material that is not known to exist in nature. A recombinant molecule can be produced by any of the well-known techniques available in the field of recombinant technology, including, but not limited to, polymerase chain reaction (PCR), gene splicing (e.g., using restriction endonucleases), and solid state synthesis of nucleic acid molecules, peptides, or proteins.

An “isolated” polypeptide, protein, peptide, or nucleic acid is a molecule that has been removed from its natural environment. It is also to be understood that “isolated” polypeptides, proteins, peptides, or nucleic acids may be formulated with excipients such as diluents or adjuvants and still be considered isolated.

As used herein, the terms “regulatory sequences,” “regulatory elements,” and “control elements” are interchangeable and refer to polynucleotide sequences that are upstream (5′ non-coding sequences), within, or downstream (3′ non-translated sequences) of a polynucleotide target to be expressed. Regulatory sequences influence, for example, the timing of transcription, amount or level of transcription, RNA processing or stability, and/or translation of the related structural nucleotide sequence. Regulatory sequences may include activator binding sequences, enhancers, introns, polyadenylation recognition sequences, promoters, repressor binding sequences, stem-loop structures, translational initiation sequences, translation leader sequences, transcription termination sequences, translation termination sequences, primer binding sites, and the like.

As used herein, “promoter,” “promoter sequence,” or “promoter region” refers to a DNA regulatory region/sequence capable of binding RNA polymerase and involved in initiating transcription of a downstream coding or non-coding sequence. In some examples, the promoter sequence includes the transcription initiation site and extends upstream to include the minimum number of bases or elements necessary to initiate transcription at levels detectable above background. In some embodiments, the promoter sequence includes a transcription initiation site, as well as protein binding domains responsible for the binding of RNA polymerase. Eukaryotic promoters will often, but not always, contain “TATA” boxes and “CAT” boxes.

“Constitutive promoters” are typically active, i.e., promote transcription, under most conditions. “Inducible promoters” are typically active only under certain conditions, such as in the presence of a given molecule factor (e.g., IPTG) or a given environmental condition (e.g., particular CO2 concentration, nutrient levels, light, heat). In the absence of that condition, inducible promoters typically do not allow significant or measurable levels of transcriptional activity. For example, inducible promoters may be induced according to temperature, pH, a hormone, a metabolite (e.g., lactose, mannitol, an amino acid), light (e.g., wavelength specific), osmotic potential (e.g., salt-induced), heavy metal, or an antibiotic. Numerous standard inducible promoters will be known to one of skill in the art.

As used herein the term “operably linked” refers to polynucleotide sequences or amino acid sequences placed into a functional relationship with one another. For instance, a promoter or enhancer is operably linked to a coding sequence if it regulates, or contributes to the modulation of, the transcription of the coding sequence. Operably linked DNA sequences encoding regulatory sequences are typically contiguous to the coding sequence. However, enhancers can function when separated from a promoter by up to several kilobases or more. Accordingly, some polynucleotide elements may be operably linked but not contiguous. Coding sequences can be operably-linked to regulatory sequences in sense or antisense orientation.

The terms “vector” and “plasmid” are used interchangeably and as used herein refer to a polynucleotide vehicle to introduce genetic material into a cell. Vectors can be linear or circular. Vectors can integrate into a target genome of a host cell or replicate independently in a host cell. Vectors can comprise, for example, an origin of replication, a multicloning site, and/or a selectable marker. An expression vector typically comprises an expression cassette. Vectors and plasmids include, but are not limited to, integrating vectors, prokaryotic plasmids, eukaryotic plasmids, plant synthetic chromosomes, episomes, viral vectors, cosmids, and artificial chromosomes. The term “vector” also includes both viral and nonviral means for introducing a nucleic acid molecule into a cell in vitro, in vivo, or ex vivo. Vectors may be introduced into the desired host cells by well-known methods, including, but not limited to, transfection, transduction, cell fusion, and lipofection. Vectors can comprise various regulatory elements including promoters.

The term “expression cassette” refers to a polynucleotide construct, generated recombinantly or synthetically, comprising regulatory sequences operably linked to a selected polynucleotide to facilitate expression of the selected polynucleotide in a host cell. For example, the regulatory sequences can facilitate transcription of the selected polynucleotide in a host cell, or transcription and translation of the selected polynucleotide in a host cell. An expression cassette can, for example, be integrated in the genome of a host cell or be present in an expression vector.

As used herein, the term “expression” refers to transcription of a polynucleotide from a DNA template, resulting in, for example, an mRNA or other RNA transcript (e.g., non-coding, such as structural or scaffolding RNAs). The term further refers to the process through which transcribed mRNA is translated into peptides, polypeptides, or proteins. Transcripts and encoded polypeptides may be referred to collectively as “gene product.” Expression may include splicing the mRNA in a eukaryotic cell, if the polynucleotide is derived from genomic DNA.

“Transfection” as used herein means the introduction of an exogenous nucleic acid molecule, including a vector, into a cell. A “transfected” cell comprises an exogenous nucleic acid molecule inside the cell and a “transformed” cell is one in which the exogenous nucleic acid molecule within the cell induces a phenotypic change in the cell. The transfected nucleic acid molecule can be integrated into the host cell's genomic DNA and/or can be maintained by the cell, temporarily or for a prolonged period of time, extra-chromosomally. Host cells or organisms that express exogenous nucleic acid molecules or fragments are referred to as “recombinant,” “transformed,” or “transgenic” organisms. In some embodiments, the invention is directed to a host cell comprising any of the expression vectors described herein, e.g., an expression vector comprising a polynucleotide encoding a Cas protein or variant thereof. In some embodiments, the invention is directed to a host cell comprising an expression vector comprising a polynucleotide encoding a Cas3, Cas9 or Cas10 protein or variant thereof.

In some embodiments, the alteration results in reduced expression of the target polynucleotide sequence. The terms “decrease,” “reduced,” “reduction,” and “decrease” are all used herein generally to mean a decrease by a statistically significant amount. However, for avoidance of doubt, decrease,” “reduced,” “reduction,” “decrease” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 10%, or at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The terms “inactivate” or “inactivation” are used herein to generally mean that the expression of a gene of interest is reduced as compared to a reference level or not expressed in a functional or active protein form. The terms “partially inactivate” or “partial inactivation” refer to an expression of the gene of interest that is reduced but not eliminated as compared to a reference level, or that a percentage of the proteins expressed by the gene still retain their activity and function. The terms “fully inactivate” or “full inactivation” as used herein mean that the gene of interest does not express any protein or all of the expressed proteins encoded by the gene of interest are inactive and nonfunctional.

As used herein, the term “exogenous” in intended to mean that the referenced molecule or the referenced polypeptide is introduced into the cell of interest. The polypeptide can be introduced, for example, by introduction of an encoding nucleic acid into the genetic material of the cells such as by integration into a chromosome or as non-chromosomal genetic material such as a plasmid or expression vector. Therefore, the term as it is used in reference to expression of an encoding nucleic acid refers to introduction of the encoding nucleic acid in an expressible form into the cell.

The term “endogenous” refers to a referenced molecule or polypeptide that is present in the cell. Similarly, the term when used in reference to expression of an encoding nucleic acid refers to expression of an encoding nucleic acid contained within the cell and not exogenously introduced.

As used herein the term “modification” refers to an alteration that physically differentiates the modified molecule from the parent molecule. In one embodiment, a variant polypeptide includes one or more modifications that differentiates the function of the variant polypeptide from the unmodified polypeptide. For example, an amino acid change in a variant polypeptide affects its receptor binding profile. In other embodiments, a variant polypeptide comprises substitution, deletion, or insertion modifications, or combinations thereof. In another embodiment, a variant polypeptide includes one or more modifications that increases its affinity for a receptor compared to the affinity of the unmodified polypeptide. In one embodiment, a variant polypeptide includes one or more substitutions, insertions, or deletions relative to a corresponding native or parent sequence. In certain embodiments, a variant polypeptide includes 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31 to 40, 41 to 50, or 51 or more modifications.

The terms “inhibitors,” “activators,” and “modulators” as used herein refer to agents that affect a function or expression of a biologically-relevant molecule. The term “modulator” includes both inhibitors and activators. They may be identified using in vitro and in vivo assays for expression or activity of a target molecule. In some cases, “inhibitors” are agents that, e.g., inhibit expression or bind to target molecules or proteins. They may partially or totally block stimulation or have protease inhibitor activity. They may reduce, decrease, prevent, or delay activation, including inactivation, desensitization, or down regulation of the activity of the described target protein. Modulators may be antagonists or agonists of the target molecule or protein. In some cases, “activators” are agents that, e.g., induce or activate the function or expression of a target molecule or protein. They may bind to, stimulate, increase, open, activate, or facilitate the target molecule activity. Activators may be agonists of the target molecule or protein. The terms “subject” and “individual” are used interchangeably herein, and refer to an animal, for example, a human from whom cells can be obtained and/or to whom treatment, including prophylactic treatment, with the cells as described herein, is provided. For treatment of those infections, conditions or disease states which are specific for a specific animal such as a human subject, the term subject refers to that specific animal. The “non-human animals” and “non-human mammals” as used interchangeably herein, includes mammals such as rats, mice, rabbits, sheep, cats, dogs, cows, pigs, and non-human primates. The term “subject” also encompasses any vertebrate including but not limited to mammals, reptiles, amphibians and fish. However, advantageously, the subject is a mammal such as a human, or other mammals such as a domesticated mammal, e.g. dog, cat, horse, and the like, or production mammal, e.g. cow, sheep, pig, and the like.

The terms “homologs” and “homologous sequences” refer to bioactive molecules that are similar to a reference molecule at the nucleotide sequence, peptide sequence, functional, or structural level. Homologs may include sequence derivatives that share a certain percent identity with the reference sequence. Thus, in one embodiment, homologous or derivative sequences share at least a 70 percent sequence identity. In a specific embodiment, homologous or derivative sequences share at least an 80 or 85 percent sequence identity. In a specific embodiment, homologous or derivative sequences share at least a 90 percent sequence identity. In a specific embodiment, homologous or derivative sequences share at least a 95 percent sequence identity. In a more specific embodiment, homologous or derivative sequences share at least an 50, 55, 60, 65, 70, 75, 85, 86, 87, 88, 89, 90, 91, 92, 93, 94, 95, 96, 97, 98, or 99 percent sequence identity. In one embodiment, homologous or derivative sequences share 100% sequence identity to the reference sequence. Homologous or derivative nucleic acid sequences may also be defined by their ability to remain bound to a reference nucleic acid sequence under high stringency hybridization conditions. Homologs having a structural or functional similarity to a reference molecule may be chemical derivatives of the reference molecule. Methods of detecting, generating, and screening for structural and functional homologs as well as derivatives are known in the art.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The term “sequence identity” or “% identity” in the context of two or more nucleic acid or polypeptide sequences, refers to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared. For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al, infra).

One example of an algorithm that is suitable for determining percent sequence identity and sequence similarity is the BLAST algorithm, which is described in Altschul et al, J. Mol. Biol. 215:403-410 (1990). Software for performing BLAST analyses is publicly available through the National Center for Biotechnology Information.

The practice of the particular embodiments will employ, unless indicated specifically to the contrary, conventional methods of chemistry, biochemistry, organic chemistry, molecular biology, microbiology, recombinant DNA techniques, genetics, immunology, and cell biology that are within the skill of the art, many of which are described below for the purpose of illustration. Such techniques are explained fully in the literature. See, e.g., Sambrook, et al., Molecular Cloning: A Laboratory Manual (3rd Edition, 2001); Sambrook, et al., Molecular Cloning: A Laboratory Manual (2nd Edition, 1989); Maniatis et al., Molecular Cloning: A Laboratory Manual (1982); Ausubel et al., Current Protocols in Molecular Biology (John Wiley and Sons, updated July 2008); Short Protocols in Molecular Biology: A Compendium of Methods from Current Protocols in Molecular Biology, Greene Pub. Associates and Wiley-Interscience; Glover, DNA Cloning: A Practical Approach, vol. I & II (IRL Press, Oxford, 1985); Anand, Techniques for the Analysis of Complex Genomes, (Academic Press, New York, 1992); Transcription and Translation (B. Hames & S. Higgins, Eds., 1984); Perbal, A Practical Guide to Molecular Cloning (1984); Harlow and Lane, Antibodies, (Cold Spring Harbor Laboratory Press, Cold Spring Harbor, N.Y., 1998) Current Protocols in Immunology Q. E. Coligan, A. M. Kruisbeek, D. H. Margulies, E. M. Shevach and W. Strober, eds., 1991); Annual Review of Immunology; as well as monographs in journals such as Advances in Immunology.

It is noted that the claims may be drafted to exclude any optional element. As such, this statement is intended to serve as antecedent basis for use of such exclusive terminology as “solely,” “only,” and the like in connection with the recitation of claim elements, or use of a “negative” limitation. As will be apparent to those of skill in the art upon reading this disclosure, each of the individual embodiments described and illustrated herein has discrete components and features readily separated from or combined with the features of any of the other several embodiments without departing from the scope or spirit of the invention. Any recited method may be carried out in the order of events recited or in any other order that is logically possible. Although any methods and materials similar or equivalent to those described herein may also be used in the practice or testing of the invention, representative illustrative methods and materials are now described.

As described in the present invention, the following terms will be employed, and are defined as indicated below.

Before the invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention. Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number, which, in the context presented, provides the substantial equivalent of the specifically recited number.

All publications, patents, and patent applications cited in this specification are incorporated herein by reference to the same extent as if each individual publication, patent, or patent application were specifically and individually indicated to be incorporated by reference. Furthermore, each cited publication, patent, or patent application is incorporated herein by reference to disclose and describe the subject matter in connection with which the publications are cited. The citation of any publication is for its disclosure prior to the filing date and should not be construed as an admission that the invention described herein is not entitled to antedate such publication by virtue of prior invention. Further, the dates of publication provided might be different from the actual publication dates, which may need to be independently confirmed.

### III. DETAILED DESCRIPTION OF THE EMBODIMENTS

A. Engineered Histocompatible Cells

As will be appreciated, the methods provided herein can be conducted on any type of nucleated cell. In some embodiments, the cell (e.g., unmodified cell) is a type A cell. In some embodiments, the cell is a type B cell. In some embodiments, the cell is a type AB cell. In some embodiments, the cell is a type O cell. In some embodiments, the cell is a type O cell. In some embodiments, the cell is a type A Rh+ (e.g., A+) cell. In some embodiments, the cell is a type A RH− (e.g., A−) cell. In some embodiments, the cell is a type B Rh+(e.g., B+) cell. In some embodiments, the cell is a type B Rh− (e.g., B−) cell. In some embodiments, the cell is a type AB Rh+(e.g., AB+) cell. In some embodiments, the cell is a type AB Rh− (e.g., AB−) cell. In some embodiments, the cell is a type O Rh+(e.g., O+) cell. In some embodiments, the cell (e.g., unmodified cell) is a type hh cell and has a Bombay phenotype.

In some embodiments, engineered histocompatible cells outlined herein are produced using gene editing to modify one or more of the cells' histo-blood antigens including A, B, H, and Rh antigens. In some embodiments, an engineered histocompatible cell is generated using gene editing to modify, for instance, a type A cell to a type O cell, a type B cell to a type O cell, a type AB cell to a type O cell, a type A+ cell to a type O− cell, a type A− cell to a type O− cell, a type AB+ cell to a type O− cell, a type AB− cell to a type O− cell, a type B+ cell to a type O− cell, and a type B− cell to a type O− cell.

In some embodiments, a cell from a blood group A individual or a group A cell is genetically modified at the ABO gene to no longer produce a functional glycosyltransferase with α(1,3) N-acetylgalactosaminyltransferase activity. In some embodiments, a cell from a blood group B individual or a group B cell is genetically modified at the ABO gene to no longer produce a functional glycosyltransferase with α(1,3)galactosyltransferase activity. In some embodiments, a cell from a blood group A individual, a blood group B individual, or a blood group AB individual is engineered according to the methods described herein to produce a cell lacking ABO activity. In some instances, such a cell without ABO activity expresses or presents an unmodified H antigen.

In some embodiments, an engineered histocompatible cell comprises a gene modification in the ABO gene. In some embodiments, the gene modification affects one allele of the ABO gene. In some embodiments, the gene modification affects two alleles of the ABO gene. In some embodiments, the gene modification is an insertion, deletion, or disruption of the ABO gene. In some embodiments, the gene modification is a homozygous modification of the ABO gene. In some embodiments, the gene modification is a heterozygous modification of the ABO gene.

In some embodiments, an engineered histocompatible cell comprises a gene modification in the FUT1 gene. In some embodiments, the gene modification affects one allele of the FUT1 gene. In some embodiments, the gene modification affects two alleles of the FUT1 gene. In some embodiments, the gene modification is an insertion, deletion, or disruption of the FUT1 gene. In some embodiments, the gene modification is a homozygous modification of the FUT1 gene. In some embodiments, the gene modification is a heterozygous modification of the FUT1 gene.

In some embodiments, an engineered histocompatible cell comprises a gene modification in the RHD gene. In some embodiments, the gene modification affects one allele of the RHD gene. In some embodiments, the gene modification affects two alleles of the RHD gene. In some embodiments, the gene modification is an insertion, deletion, or disruption of the RHD gene. In some embodiments, the gene modification is a homozygous modification of the RHD gene. In some embodiments, the gene modification is a heterozygous modification of the RHD gene.

In some embodiments, an engineered histocompatible cell comprises a gene modification of the ABO gene such that a functional ABO gene product is not generated by the cell. In some embodiments, an engineered histocompatible cell comprises a gene modification of the FUT1 gene such that a functional FUT1 gene product is not generated by the cell. In some embodiments, an engineered histocompatible cell comprises a gene modification of the RHD gene such that a functional RHD gene product is not generated by the cell.

In some embodiments, an engineered histocompatible cell comprises a gene modification in one or more genes encoding histocompatibility determinants. In some embodiments, the cell comprises a gene modification of the ABO gene and a gene modification of the RHD gene. In some instances, the engineered cell is a type O negative cell.

In some embodiments, the engineered cell comprises a gene modification of the FUT1 gene and a gene modification of the RHD gene. As such, the engineered cell can be a type O negative cell.

Gene editing using a rare-cutting endonuclease such as, but not limited to Cas9 is utilized to a targeted disruption of one or more genes encoding a histocompatibility determinant, such as but not limited to, an ABO gene, a FUT1 gene, and an RHD gene.

In some instances, the targeted disruption of the ABO gene targets any one of its coding exons. In other instances, the targeted disruption of the FUT1 gene targets any one of its coding exons. In certain instances, the targeted disruption of the RHD gene targets any one of its coding exons. In some embodiments, the entire coding sequence or a large portion thereof of the gene is disrupted or excised. In some embodiments, insertion-deletions by way of CRISPR/Cas9 editing are introduced into the cell to disruption of the ABO gene, the RHD gene, and/or the FUT1 gene.

In some embodiments, a RNA guided-DNA nuclease is used to target the coding sequence of the ABO gene to introduce deleterious variations of the ABO gene and disruption ABO function. In other embodiments, the untranslated region, intron sequence and/or exon sequences of the ABO are targeted.

In some embodiments, the deleterious variation of the ABO gene comprises an indel. In some embodiments, the deleterious variation of the ABO gene comprises a deletion. In some embodiments, the deleterious variation of the ABO gene comprises an insertion. In some embodiments, the deleterious variation of the ABO gene comprises a frameshift mutation. In some embodiments, the deleterious variation of the ABO gene comprises a substitution. In some embodiments, the deleterious variation of the ABO gene comprises a point mutation. In some embodiments, the deleterious variation of the ABO gene reduced the expression of the gene. In some embodiments, the deleterious variation of the ABO gene comprises a loss-of-function mutation.

In some embodiments, a RNA guided-DNA nuclease is used to target the coding sequence of the FUT1 gene to introduce deleterious variations of the FUT1 gene and disruption FUT1 function. In other embodiments, the untranslated region, intron sequence and/or exon sequences of the FUT1 are targeted.

In some embodiments, the deleterious variation of the FUT1 gene comprises an indel. In some embodiments, the deleterious variation of the FUT1 gene comprises a deletion. In some embodiments, the deleterious variation of the FUT1 gene comprises an insertion. In some embodiments, the deleterious variation of the FUT1 gene comprises a frameshift mutation. In some embodiments, the deleterious variation of the FUT1 gene comprises a substitution. In some embodiments, the deleterious variation of the FUT1 gene comprises a point mutation. In some embodiments, the deleterious variation of the FUT1 gene reduced the expression of the gene. In some embodiments, the deleterious variation of the FUT1 gene comprises a loss-of-function mutation.

In some embodiments, a RNA guided-DNA nuclease is used to target the coding sequence of the RHD gene to introduce deleterious variations of the RHD gene and disruption RHD function. In other embodiments, the untranslated region, intron sequence and/or exon sequences of the RHD are targeted.

In some embodiments, the deleterious variation of the RHD gene comprises an indel. In some embodiments, the deleterious variation of the RHD gene comprises a deletion. In some embodiments, the deleterious variation of the RHD gene comprises an insertion. In some embodiments, the deleterious variation of the RHD gene comprises a frameshift mutation. In some embodiments, the deleterious variation of the RHD gene comprises a substitution. In some embodiments, the deleterious variation of the RHD gene comprises a point mutation. In some embodiments, the deleterious variation of the RHD gene reduced the expression of the gene. In some embodiments, the deleterious variation of the RHD gene comprises a loss-of-function mutation.

In some embodiments, the histocompatibility of the cells is determined using a complement mediated cell killing assay. A non-limiting example of such as assay is an XCelligence SP platform (ACEA BioSciences).

The histocompatible cells of the present invention can be produced from cells including cells in culture, for example cultured mammalian cells, e.g., cultured human cells. In some embodiments, the cells are stem cells. In some embodiments, the stem cells retain pluripotency. In some embodiments, the stem cells retain differentiation potential. In some embodiments, the cells are pluripotent stem cells or non-pluripotent stem cells, multipotent stem cells or non-multipotent stem cells, progenitor cells or non-progenitor cells, differentiated cells or non-differentiated cell, and the like. In some embodiments, the cells are primary cells or cell lines (e.g., a mammalian cell lines including human cell lines).

In some embodiments, the histocompatible cells of the present invention are produced from cells that are pluripotent stem cells, induced pluripotent stem cells, embryonic stem cells (such as RUES2 or H9 cells), adult stem cells, and multipotent stem cells. In certain embodiments, the cells are differentiated cells derived or generated from pluripotent stem cells, induced pluripotent stem cells, embryonic stem cells (such as RUES2 or H9 cells), adult stem cells, and multipotent stem cells. Such differentiated cells can be a cell type of any tissue or organ of the body. In some embodiments, the cells are any type that is useful for cell-based therapies.

In some embodiments, the histocompatible cells of the present invention are produced from cells are from any organ or tissue of the body, including but not limited to, epithelial, connective, muscular, or nervous tissue or cells, and combinations thereof. In some instances, the cells from any eukaryotic (e.g., mammalian) organ system, for example, from the cardiovascular system (heart, vasculature); digestive system (esophagus, stomach, liver, gallbladder, pancreas, intestines, colon, rectum and anus); endocrine system (hypothalamus, pituitary gland, pineal body or pineal gland, thyroid, parathyroids, adrenal glands); excretory system (kidneys, ureters, bladder); lymphatic system (lymph, lymph nodes, lymph vessels, tonsils, adenoids, thymus, spleen); integumentary system (skin, hair, nails); muscular system (e.g., skeletal muscle); nervous system (brain, spinal cord, nerves); reproductive system (ovaries, uterus, mammary glands, testes, vas deferens, seminal vesicles, prostate); respiratory system (pharynx, larynx, trachea, bronchi, lungs, diaphragm); skeletal system (bone, cartilage), and combinations thereof.

In some embodiments, the histocompatible cells of the present invention are produced from cells are from a highly mitotic tissue (e.g., a highly mitotic healthy tissue, such as epithelium, embryonic tissue, bone marrow, intestinal crypts).

In some embodiments, the histocompatible cells of the present invention are produced from adult stem cells, which are stem cells from any organ or tissue of the body, including but not limited to, epithelial, connective, muscular, or nervous tissue or cells, and combinations thereof. In some instances, the histocompatible cells of the present invention are produced from cells from any eukaryotic (e.g., mammalian) organ system, for example, from the cardiovascular system (heart, vasculature); digestive system (esophagus, stomach, liver, gallbladder, pancreas, intestines, colon, rectum and anus); endocrine system (hypothalamus, pituitary gland, pineal body or pineal gland, thyroid, parathyroids, adrenal glands); excretory system (kidneys, ureters, bladder); lymphatic system (lymph, lymph nodes, lymph vessels, tonsils, adenoids, thymus, spleen); integumentary system (skin, hair, nails); muscular system (e.g., skeletal muscle); nervous system (brain, spinal cord, nerves); reproductive system (ovaries, uterus, mammary glands, testes, vas deferens, seminal vesicles, prostate); respiratory system (pharynx, larynx, trachea, bronchi, lungs, diaphragm); skeletal system (bone, cartilage), and combinations thereof. In many embodiments, the multipotent stem cells are cells that have the capacity to self-renew and develop (differentiated) into two or more specialized cell types present in a specific organ or tissue of the body, as described herein.

In some embodiments, the histocompatible cells of the present invention are produced from cells that are progenitor cells, e.g., bone marrow stromal cells, marrow derived adult progenitor cells (MAPCs), endothelial progenitor cells (EPC), blast cells, intermediate progenitor cells formed in the subventricular zone, neural stem cells, muscle stem cells, satellite cells, liver stem cells, hematopoietic stem cells, bone marrow stromal cells, epidermal stem cells, embryonic stem cells (such as RUES2 or H9 cells), mesenchymal stem cells, umbilical cord stem cells, precursor cells, muscle precursor cells, myoblast, cardiomyoblast, neural precursor cells, glial precursor cells, neuronal precursor cells, and hepatoblasts.

B. Methods of Modifying Expression of Histocompatible-Blood Determinants

Provided herein are methods of modifying nucleic acid sequences within cells to generate genetically modified cells. Exemplary technologies for such modification include homologous recombination, knock-in technology, zinc finger nucleases (ZFNs), transcription activator-like effector nucleases (TALENs), CRISPR/Cas9, and other site-specific nuclease technologies. These techniques enable double-strand DNA breaks at desired locus sites. These controlled double-strand breaks promote homologous recombination at the specific locus sites. This process focuses on targeting specific sequences of nucleic acid molecules, such as chromosomes, with endonucleases that recognize and bind to the sequences and induce a double-stranded break in the nucleic acid molecule. The double-strand break is repaired either by an error-prone non-homologous end-joining (NHEJ) or by homologous recombination (HR).

In one embodiment, the cells are manipulated using CRISPR/Cas technologies as is known in the art. There are a large number of techniques based on CRISPR, see, e.g., Doudna and Charpentier, Science, doi:10.1126/science.1258096. CRISPR techniques and kits are sold commercially.

In some embodiments, the cells of the invention are made using Transcription Activator-Like Effector Nucleases (TALEN) methodologies. TALEN are restriction enzymes combined with a nuclease that can be engineered to bind to and cut practically any desired DNA sequence. TALEN kits are sold commercially.

In some embodiments, the cells are manipulated using zinc finger nuclease technologies. Zinc finger nucleases are artificial restriction enzymes generated by fusing a zinc finger DNA-binding domain to a DNA-cleavage domain. Zinc finger domains can be engineered to target specific desired DNA sequences and this enables zinc-finger nucleases to target unique sequences within complex genomes. By taking advantage of endogenous DNA repair machinery, these reagents can be used to precisely alter the genomes of higher organisms, similar to CRISPR and TALENs.

As will be appreciated by those in the art, a number of different techniques can be used to engineer cells to be blood histocompatible cells, and in some embodiments, also hypo-immunogenic as outlined herein.

1. Genetic Modifications of ABO Gene

Provided herein are methods for producing a histo-blood type O cell from any other histo-blood type cell using gene editing tools such as but not limited to CRISPR/Cas, TALE-nucleases, zinc finger nucleases, other viral based gene editing system, or RNA interference, such as CRISPR/Cas9 gene editing.

In some embodiments, a Cas9 editing system is used to target a sequence of the ABO gene to introduce an insertion or deletion into the gene to disrupt its function, and in some instances, to render it inactive. In some embodiments, a single guide RNA is used. In some embodiments, dual guide RNAs are used. In some embodiments, any one of the gRNA target sequences of Tables 1, A, B, and C, are used. In some instances, more than one gRNA target sequence of Tables 1, A, B, and C is used for gene editing.

In some embodiments, a frame-shift deletion, e.g., a frame-shift single base deletion is introduced into the ABO gene to inactive the gene. In some instances, the deletion is produced using a Cas9 editing system and a guide RNA target sequence of Table 1. In some embodiments, an engineered histocompatible cell comprises a homozygous frame-shift deletion of the ABO gene. In some embodiments, an insertion-deletion (indel) is introduced in a coding exon, such as exon 1. In some embodiments, an insertion-deletion (indel) is introduced in a coding exon, such as exon 2. In some embodiments, an insertion-deletion (indel) is introduced in a coding exon, such as exon 8. In some instances, the indel is produced using a Cas9 editing system and a guide RNA target sequence of Table 1. In some embodiments, an engineered histocompatible cell comprises a homozygous indel of the ABO gene.

TABLE 1
Exemplary ABO gRNA target sequences

| SEQ ID NO: | Guide RNA name | Position | Strand | Sequence | PAM |
|---|---|---|---|---|---|
| SEQ ID NO: 1 | ABO Exon #1 gRNA | 36 | 1 | GGCCAGCGTCCGCAACACCT | CGG |
| SEQ ID NO: 2 | ABO Exon #2 gRNA | 13066 | -1 | GGATCATAGGTCGAAGTGCG | TGG |
| SEQ ID NO: 3 | ABO Exon 6-7 gRNA 1 | 17691 | -1 | GCCAGCCAAGGGGTACCACG | AGG |
| SEQ ID NO: 4 | ABO Exon 6-7 gRNA 2 | 17697 | 1 | GATGTCCTCGTGGTACCCCT | TGG |
| SEQ ID NO: 5 | ABO Exon 6-7 gRNA 3 | 17715 | -1 | AATGTGCCCTCCCAGACAAT | GGG |
| SEQ ID NO: 15 | ABO Exon 6-7 gRNA 4 | 17675 | 1 | TCTCTCCATGTGCAGTAGGA | AGG |
| SEQ ID NO: 16 | ABO Exon 6-7 gRNA 5 | 17687 | | CAGTAGGAAGGATGTCCTCG | TGG |

In some embodiments, an insertion-deletion is generated to introduce a 258delG mutation in exon 6 of the ABO gene by gene editing, e.g., homology-directed repair (HDR). In some instances, ABO 258delG variant is knocked into the ABO gene. In some embodiments, a gRNA target sequence for a specific ABO Cas9 protospacer adjacent motif (PAM) and a HDR donor template encoding the 258delG variation are used to produce an engineered type O cell. In some embodiments, the ABO exon 6-7 gRNA target sequence (ABO exon 6-7 gRNA 1) and the donor template (ABO 258delG donor template guide 1) of Table 2 are used. The donor template includes the 258delG variant and a silent mutation that destroys the PAM site on the reverse strand. In some embodiments, the donor template includes two homology arms, each containing about 10 nt to 1 Kb (e.g., 20, nt, 30 nt, 40 nt, or 50 nt to 1 Kb) of homologous sequence

In some embodiments, the ABO exon 6-7 gRNA target sequence (ABO exon 6-7 gRNA 2) and the donor template (ABO 258delG donor template guide 2) of Table 2 are used. The donor template includes the 258delG variant and a silent mutation that introduces 2 mismatches in the gRNA sequence to prevent continual editing of the repaired genomic sequence. In some embodiments, the donor template includes two homology, each containing about 10 nt to 1 Kb (e.g., 20, nt, 30 nt, 40 nt, or 50 nt to 1 Kb) of homologous sequence.

In some embodiments, the ABO exon 6-7 gRNA target sequence (ABO exon 6-7 gRNA 3) and the donor template (ABO 258delG donor template guide 3) of Table 2 are used. The donor template includes the 258delG variant and a silent mutation that introduces 2 mismatches in the gRNA sequence to prevent continual editing of the repaired genomic sequence. In some embodiments, the donor template includes two homology arms, each containing about 10 nt to 1 Kb (e.g., 20, nt, 30 nt, 40 nt, or 50 nt to 1 Kb) of homologous sequence.

In some embodiments, the ABO exon 6-7 gRNA target sequence (ABO exon 6-7 gRNA 4) and the donor template (ABO 258delG donor template guide 4) of Table 2 are used. The donor template includes the 258delG variant and a silent mutation that introduces 2 mismatches in the gRNA sequence to prevent continual editing of the repaired genomic sequence. In some embodiments, the donor template includes two homology arms, each containing about 10 nt to 1 Kb (e.g., 20, nt, 30 nt, 40 nt, or 50 nt to 1 Kb) of homologous sequence.

TABLE 2
Exemplary ABO HDR Donor Templates

| SEQ ID | Guide | HDR Template | |
|---|---|---|---|
| NO: | RNA name | name | HDR sequence 5′→3′ |
| SEQ ID | ABO Exon | ABO 258delG | TGGGGGCGGCCGTGTGCCAGAGGCGCATGTGGGTGGCACCC |
| NO: 6 | 6-7 gRNA | donor template | TGCCAGCTCCATGTGACCGCACGCCTCTCTCCATGTGCAGTAG |
| | 1 | guide 1 | GAAGGATGTTCTCGTGTACCCCTTGGCTGGCTCCCATTGTCTG |
| | | | GGAGGGCACATTCAACATCGACATCCTCAACGAGCAGTTCAG |
| | | | GCTCCAGAACACCACCATTGGGTTAACTGTG |
| SEQ ID | ABO Exon | ABO 258delG | TGGGGGCGGCCGTGTGCCAGAGGCGCATGTGGGTGGCACCC |
| NO: 7 | 6-7 gRNA | donor template | TGCCAGCTCCATGTGACCGCACGCCTCTCTCCATGTGCAGTAG |
| | 2 | guide 2 | GAAGGATGTCCTCGTAAACCCCTTGGCTGGCTCCCATTGTCTG |
| | | | GGAGGGCACATTCAACATCGACATCCTCAACGAGCAGTTCAG |
| | | | GCTCCAGAACACCACCATTGGGTTAACTGTG |
| SEQ ID | ABO Exon | ABO 258delG | TGGGGGCGGCCGTGTGCCAGAGGCGCATGTGGGTGGCACCC |
| NO: 8 | 6-7 gRNA | donor template | TGCCAGCTCCATGTGACCGCACGCCTCTCTCCATGTGCAGTAG |
| | 3 | guide 3 | GAAGGATGTCCTCGTGTACCCCTTGGCTGGCTCCCATTGTTTG |
| | | | GGAGGGCACTTTCAACATCGACATCCTCAACGAGCAGTTCAG |
| | | | GCTCCAGAACACCACCATTGGGTTAACTGTG |
| SEQ ID | ABO Exon | ABO 258delG | CTCCATGTGACCGCACGCCTCTCTCCATGTGCAGTAGGA |
| NO: 19 | 6-7 gRNA | donor template | AGGATGTCCTCGTGTACCCCTTGGCTGGCTCCCATTGTCT |
| | 4 | guide 4 | GGGAGGGCACATTCAACATCGAC |

In some embodiments, an engineered histocompatible cell comprises a homozygous 258delG mutation of the ABO gene.

2. Genetic Modifications of RHD Gene

Provided herein are methods for producing a Rh negative cell by gene editing the RHD gene using gene editing tools such as but not limited to CRISPR/Cas, TALE-nucleases, zinc finger nucleases, other viral based gene editing system, or RNA interference. In some embodiments, the gene editing targets the coding sequence of the RHD gene. In some instances, the cells do not generate a functional RHD gene product. In the absence of the RHD gene product, the cells completely lack an Rh blood group antigen.

In some embodiments, a Cas9 editing system is used to target a sequence of the RHD gene to introduce an insertion or deletion into the gene to disrupt its function, and in some instances, to render it inactive. In some embodiments, a single guide RNA is used. In some embodiments, dual guide RNAs are used. In some embodiments, any one of the gRNA target sequences of Tables 3, D, E, and F are used. In some instances, more than one gRNA target sequences of Tables 3, D, E, and F are used for gene editing.

In some embodiments, a frame-shift insertion-deletion is introduced in any coding sequence of the gene. In some embodiments, a modification within the UTRs, introns, or exons of the gene is added to disrupt the function of the RHD gene. In some embodiments, CRISPR/Cas9 editing comprising any one or more of the gRNA target sequences of Tables 3, D, E, and F are utilized.

In some embodiments, a modification is introduced into the RHD gene to inactivate the gene. In some embodiments, coding exons such as exon 1 or exon 2 of the RHD gene are targeted. In some embodiments, coding exon 4 of the RHD gene are targeted. In some embodiments, coding exon 5 of the RHD gene are targeted. In some embodiments, coding exon 6 of the RHD gene are targeted. In some embodiments, coding exon 7 of the RHD gene are targeted. In some embodiments, coding exon 8 of the RHD gene are targeted. In some instances, a deletion is produced using a Cas9 editing system and a guide RNA target sequence targeting a sequence at the 5′ of the RHD gene and a guide RNA target sequence to an exon such as but not limited to exon 8. In some embodiments, one gRNA target sequence is the RHD 5′ UTR guide 1 of Table 3 and one gRNA target sequence is the RHD exon 8 guide 1 of Table 3. In some embodiments, an engineered histocompatible cell comprises a homozygous modification of the RHD gene, thereby inactivating the gene.

TABLE 3
Exemplary RHD gRNA target sequences

| SEQ ID NO: | Guide RNA name | Position | Strand | Sequence | PAM |
|---|---|---|---|---|---|
| SEQ ID NO: 9 | RHD gRNA 1 | 25290638 | -1 | CACCGACAAAGCACTCATGG | TGG |
| SEQ ID NO: 10 | RHD gRNA 2 | 25284571 | 1 | TGGCCAAGATCTGACCGTGA | TGG |
| SEQ ID NO: 11 | RHD Exon 8 guide 1 | 25307729 | 1 | GGAGGCGCTGCGGTTCCTAC | CGG |
| SEQ ID NO: 12 | RHD 5′ UTR guide 1 | 25272403 | -1 | TGGTTGTGCTGGCCTCTCTA | TGG |

In some embodiments, the gRNA target sequence is to exon 1 or exon 2 of the RHD gene. In some embodiments, the gRNA target sequence is a gRNA of Table 3 that induces a frameshift mutation to inactivate exon 1 or exon 2.

3. Genetic Modifications of FUT1 Gene

Provided herein are methods for producing a histo-blood type O cell from any other histo-blood type cell by gene editing the FUT1 gene using gene editing tools such as but not limited to CRISPR-Cas9, TALE-nucleases, zinc finger nucleases, other viral based gene editing system, or RNA interference. In some embodiments, the gene editing targets the coding sequence of the FUT1 gene. In some instances, the cells do not generate a functional FUT1 gene product. In the absence of FUT1, the cells completely lack the H antigen and have a Bombay histo-blood type (hh).

In some embodiments, a Cas9 editing system is used to target a sequence of the FUT1 gene to introduce an insertion or deletion into the gene to disrupt its function, and in some instances, to render it inactive. In some embodiments, a single guide RNA is used. In some embodiments, dual guide RNAs are used. In some embodiments, any one of the gRNA target sequences of Tables 4, G, H, and I are used. In some instances, more than one gRNA target sequence of Table 4, G, H, and I is used for gene editing.

In some embodiments, a modification is introduced into the FUT1 gene to inactive the gene. In some embodiments, coding exons such as exon 4 of the FUT1 gene are targeted. In some instances, the deletion is produced using a Cas9 editing system and a guide RNA target sequence of Table 4. In some embodiments, an engineered histocompatible cell comprises a homozygous modification of the FUT1 gene, thereby inactivating the gene.

TABLE 4
Exemplary FUT1 gRNA target sequences

| SEQ ID NO | Guide RNA name | Position | Strand | Sequence | PAM |
|---|---|---|---|---|---|
| SEQ ID NO: 13 | FUT1 Exon 4 guide | 4226 | -1 | GGTCTGGACACAGGATCGAC | AGG |
| SEQ ID NO: 14 | FUT1 Exon 4 guide | 4367 | -1 | GATTACCAAACCGGCCATTG | GGG |
| | 2 | | | | |
| SEQ ID NO: 20 | FUT1 Exon 4 guide | 4570 | 1 | CTGGATGTCGGAGGAGTACG | CGG |
| | 3 | | | | |

In some embodiments, a frame-shift insertion-deletion is introduced in any coding sequence of the gene. In some embodiments, a modification within the UTRs, introns, or exons of the gene is added to disrupt the function of the FUT1 gene. In some embodiments, CRISPR-Cas9 editing comprising any one or more of the gRNA target sequences of Tables 4, G, H, and I are utilized.

In some embodiments, a first gene modification disrupts the function of the ABO gene and a second gene modification disrupts the function of the RHD gene. In some embodiments, a first gene modification disrupts the function of the FUT1 gene and a second gene modification disrupts the function of the RHD gene.

In general, these methods can be used individually or in combination. In some embodiments, the cells also include a B2M gene modification, a CIITA gene modification, and a CD47 transgene. In some embodiments, the cells have a B2M homozygous null genotype. In some embodiments, the cells have a CIITA homozygous null genotype. In some embodiments, the cells overexpress CD47. In some embodiments, the modified cells carry gene modifications including, but not limited to, B2M^{-/-}, CIITA^{-/-}, CD47 tg.

In some embodiments, in the generation of the hypoimmunogenic cells including hypoimmunogenic pluripotent stem cells and hypoimmunogenic induced pluripotent stem cells, CRISPR may be used to reduce the expression of active B2M and/or CIITA protein in the engineered cells, with viral techniques (e.g., lentivirus) to knock in the CD47 functionality. Also, as will be appreciated by those in the art, although one embodiment sequentially utilizes a CRISPR step to knock out B2M, followed by a CRISPR step to knock out CIITA with a final step of a lentivirus to knock in the CD47 functionality, these genes can be manipulated in different orders using different technologies.

C. Additional Modifications to Generate Hypoimmunogenic Cells

Provided herein are cells comprising a modification of one or more targeted polynucleotide sequences that regulates the expression of MHC I and/or MHC II. In some embodiments, the expression of both MHC I and MHC II is regulated by one or more targeted polynucleotides sequences such as those described herein.

In some embodiments, the cell comprises a genomic modification of one or more targeted polynucleotide sequences that regulates the expression of MHC I. In some embodiments, the cell comprises a genomic modification of one or more targeted polynucleotide sequences that regulates the expression of MHC II. In some aspects, a genetic editing system is used to modify one or more targeted polynucleotide sequences. In some embodiments, the targeted polynucleotide sequence is one or more selected from the group consisting of B2M, CIITA, and NLRC5. In certain embodiments, the genome of the cell has been altered to reduce or delete critical components of HLA expression.

In some aspects, the present disclosure provides a stem cell (e.g., pluripotent stem cell or induced pluripotent stem cell) or population thereof comprising a genome in which a gene has been edited to delete a contiguous stretch of genomic DNA, thereby reducing or eliminating surface expression of MHC class I molecules in the cell or population thereof. In some embodiments, the stem cells retain pluripotency. In some embodiments, the stem cells retain differentiation potential. In certain aspects, the present disclosure provides a stem cell (e.g., pluripotent stem cell or induced pluripotent stem cell) or population thereof comprising a genome in which a gene has been edited to delete a contiguous stretch of genomic DNA, thereby reducing or eliminating surface expression of MHC class II molecules in the cell or population thereof. In particular aspects, the present disclosure provides a stem cell (e.g., pluripotent stem cell or induced pluripotent stem cell) or population thereof comprising a genome in which one or more genes has been edited to delete a contiguous stretch of genomic DNA, thereby reducing or eliminating surface expression of MHC class I and II molecules in the cell or population thereof. The efficient interference with the expression of MHC I and/or MHC II proteins can be accomplished by one or more of the following: (1) targeting the polymorphic HLA alleles (HLA-A, -B, -C) and MHC-II genes directly; (2) removal of B2M, which will prevent surface trafficking of all MHC-I molecules; and/or (3) deletion of components of the MHC enhanceosomes, such as LRC5, RFX-5, RFX-associated ankyrin-containing protein (RFX-ANK), RFX-associated protein (RFX-AP), IRF1, NF-Y, and CIITA that are critical for HLA expression.

In certain embodiments, the expression of MHC I or MHC II is modulated by targeting and deleting a contiguous stretch of genomic DNA thereby reducing or eliminating expression of a target gene selected from the group consisting of B2M, CIITA, and NLRC5.

In some embodiments, the expression of MHC I is reduced by targeting and deleting a contiguous stretch of genomic DNA, thereby reducing or eliminating expression of HLA-A, HLA-B, and HLA-C. In some embodiments, the expression of MHC I is reduced or eliminated by way of genomic modification of the B2M gene. In some instances, the genomic modification is conducted using a CRISPR/Cas system.

In some embodiments, the cells and methods described herein include genomically editing human cells to cleave CIITA gene sequences as well as editing the genome of such cells to alter one or more additional target polynucleotide sequences such as, but not limited to, B2M and NLRC5. In some embodiments, the cells and methods described herein include genomically editing human cells to cleave B2M gene sequences as well as editing the genome of such cells to alter one or more additional target polynucleotide sequences such as, but not limited to, CIITA and NLRC5. In some embodiments, the cells and methods described herein include genomically editing human cells to cleave NLRC5 gene sequences as well as editing the genome of such cells to alter one or more additional target polynucleotide sequences such as, but not limited to, B2M and CIITA.

In some embodiments, the cell also includes a modification to increase expression of one selected from the group consisting of HLA-C, HLA-E, HLA-G, PD-L1, CTLA-4-Ig, C1-inhibitor, and IL-35. In some embodiments, the cells are generating by modulating expression of one or more genes such as RFX-5, RFX-associated ankyrin-containing protein (RFX-ANK), RFX-associated protein (RFX-AP), IRF1, NFY-A, NFY-B, NFY-C, and CIITA that are critical for HLA expression as described in WO2016/183041, incorporated by reference herein in its entirety, and specifically for the related techniques outlined therein. In additional embodiments, further modifications to the producer cells include modulating expression of one or more genes such as OX40, GITR, 4-1BB, CD28, B7-1, B7-2, ICOS, CD27, HVEM, SLAM, CD226, PD1, CTLA4, LAG3, TIGIT, TIM3, CD160, BTLA, CD244, CD30, TLT, VISTA, B7-H3, PD-L2, LFA-1, CD2, CD58, ICAM-3, TCRA, TCRB, FOXP3, HELIOS, ST2, PCSK9, CCR5, and/or APOC3 as described in WO2016/183041, incorporated by reference herein in its entirety, and specifically for the related techniques outlined therein.

In some embodiments, the cells are generated by the introduction of one or more transgenes such as PDL-1, HLA-G, CD47, CD200, FASLG, CLC21, MFGE8, and/or SERPIN B9, or a gene encoding a biologic that acts as an agonist of PDL-1, HLA-G, CD47, CD200, FASLG, CLC21, MFGE8, and/or SERPIN 9, as described in WO2018/227286, incorporated by reference herein in its entirety, and specifically for the related techniques outlined therein. In some embodiments, the cells are generated by the introduction of transgenes including PDL-1, HLA-G, and CD47. In some embodiments, the cell are generated by the introduction of transgenes including PDL-1 and HLA-G. In some embodiments, the cell are generated by the introduction of transgenes including PDL-1 and CD47. In some embodiments, the cell is generated by further introduction of one or more transgenes such as TGFβ, CD73, CD39, LAG3, IL1R2, ACKR2, TNFRSF22, TNFRSF23, TNFRS10, DAD1, and/or IFNγR1 d39, or a gene encoding a biologic that acts as an agonist of TGFβ, CD73, CD39, LAG3, IL1R2, ACKR2, TNFRSF22, TNFRSF23, TNFRS10, DAD1, and/or IFNγR1 d39, as described in WO2018/227286, incorporated by reference herein in its entirety, and specifically for the related techniques outlined therein.

In some embodiments, the cells are generated by further introducing into the cells a suicide gene that is activated by a trigger that causes the cells to die. In one embodiment, the suicide gene is a herpes simplex virus thymidine kinase gene (HSV-tk) and the trigger is ganciclovir. In another embodiment, the suicide gene is an Escherichia coli cytosine deaminase gene (EC-CD) and the trigger is 5-fluorocytosine (5-FC). In yet another embodiment, the suicide gene is an inducible Caspase protein and the trigger is a specific chemical inducer of dimerization (CID).

In some instances, a gene editing system such as the CRISPR/Cas system is used to facilitate the insertion of hypoimmunity factors, such as the hypoimmunity factors into a safe harbor locus, such as the AAVS, HPRT, CCR5 and ROSA26 locus, to actively inhibit immune rejection. In some embodiment, a hypoimmunity factor is inserted into a single safe harbor locus of the cell. In other embodiments, the hypoimmunity factors are inserted into more than one safe harbor locus. In some instances, the hypoimmunity factors are inserted into a safe harbor locus using an expression vector, e.g., a lentiviral expression vector.

Assays for to determined hypoimmunogenicity of the cells is generally described herein and in WO 2018/132783, incorporated herein by reference. For example, hypoimmunogenicity may be assayed using a number of techniques. These techniques include administration into allogeneic hosts and monitoring the T cell and/or B cell response of the host animal. T cell function can be assessed by ELISpot, ELISA, FACS, PCR, or mass cytometry (CYTOF). B cell response or antibody response can be assessed using FACS or Luminex.

In some embodiments, the hypoimmunogenic cell exhibits reduced levels of immunogenicity as compared to a reference cell, e.g., an unmodified cell or an immunogenic cell. In some embodiments, the reduced level is at least 5%, 10%, 20%, 30%, 40%, 50%, 60%, 70%, 80%, 90%, 2-fold, 3-fold, 5-fold, 10-fold, 20-fold, 50-fold, or 100-fold reduced.

In some embodiments, the hypoimmunogenic cell facilitates a reduction in macrophage phagocytosis, e.g., a reduction of 1%, 5%, 10%, 20%, 30%, 40%, 50%, 60%, 70%, 80%, 90%, or more in macrophage phagocytosis compared to a reference cell, e.g., an unmodified cell or an immunogenic cell, wherein the reduction in macrophage phagocytosis is determined by assaying the phagocytosis index in vitro.

In some embodiments, the hypoimmunogenic cell renders a reduction in cytotoxicity mediated cell lysis by PBMCs, e.g., a reduction of 1%, 5%, 10%, 20%, 30%, 40%, 50%, 60%, 70%, 80%, 90%, or more in cell lysis compared to a reference cell, e.g., an unmodified cell or an immunogenic cell.

In some embodiments, the hypoimmunogenic cell renders a reduction in NK-mediated cell lysis, e.g., a reduction of 1%, 5%, 10%, 20%, 30%, 40%, 50%, 60%, 70%, 80%, 90%, or more in NK-mediated cell lysis compared to a reference cell, e.g., an unmodified cell or an immunogenic cell, wherein NK-mediated cell lysis is assayed in vitro, by a chromium release assay or europium release assay.

In some embodiments, the hypoimmunogenic cell gives rise to a reduction in CD8+ T cell mediated cell lysis, e.g., a reduction of 1%, 5%, 10%, 20%, 30%, 40%, 50%, 60%, 70%, 80%, 90%, or more in CD8 T cell mediated cell lysis compared to a reference cell, e.g., an unmodified cell or an immunogenic cell.

In some embodiments, the hypoimmunogenic cell induces a reduction in CD4+ T cell proliferation and/or activation, e.g., a reduction of 1%, 5%, 10%, 20%, 30%, 40%, 50%, 60%, 70%, 80%, 90%, or more compared to a reference cell, e.g., an unmodified cell or an immunogenic cell, wherein CD4 T cell proliferation is assayed in vitro (e.g. co-culture assay of modified or unmodified mammalian source cell, and CD4+ T cells with CD3/CD28 Dynabeads).

In some embodiments, the hypoimmunogenic cell causes a reduction in T cell IFN-gamma secretion, e.g., a reduction of 1%, 5%, 10%, 20%, 30%, 40%, 50%, 60%, 70%, 80%, 90%, or more in T cell IFN-gamma secretion compared to a reference cell, e.g., an unmodified cell or an immunogenic cell, wherein T cell IFN-gamma secretion is assayed in vitro, e.g., by IFN-gamma ELISPOT.

In some embodiments, the hypoimmunogenic cell causes a reduction in secretion of immunogenic cytokines, e.g., a reduction of 1%, 5%, 10%, 20%, 30%, 40%, 50%, 60%, 70%, 80%, 90%, or more in secretion of immunogenic cytokines compared to a reference cell, e.g., an unmodified cell or an immunogenic cell, wherein secretion of immunogenic cytokines is assayed in vitro using ELISA or ELISpot.

In some embodiments, the hypoimmunogenic cell induces an increased secretion of an immunosuppressive cytokine, e.g., an increase of 1%, 5%, 10%, 20%, 30%, 40%, 50%, 60%, 70%, 80%, 90%, or more in secretion of an immunosuppressive cytokine compared to a reference cell, e.g., an unmodified cell or an immunogenic cell, wherein secretion of the immunosuppressive cytokine is assayed in vitro using ELISA or ELISPOT.

The cells described herein can also be assessed for immunogenicity. In some embodiments, a cell is analyzed for the presence of antibodies on the cell surface, e.g., by staining with an anti-IgM antibody. In other embodiments, immunogenicity is assessed by a PBMC cell lysis assay. In some embodiments, a cell is incubated with peripheral blood mononuclear cells (PBMCs) and then assessed for lysis of the cells by the PBMCs. In other embodiments, immunogenicity is assessed by a natural killer (NK) cell lysis assay. In some embodiments, a cell is incubated with NK cells and then assessed for lysis of the cells by the NK cells. In other embodiments, immunogenicity is assessed by a CD8+ T cell lysis assay. In some embodiments, a cell is incubated with CD8+ T cells and then assessed for lysis of the cells by the CD8+ T cells.

1. CIITA

In certain aspects, the inventions disclosed herein modulate (e.g., reduce or eliminate) the expression of MHC II genes by targeting and modulating (e.g., reducing or eliminating) Class II transactivator (CIITA) expression. In some aspects, the modulation occurs using a CRISPR/Cas system. CIITA is a member of the LR or nucleotide binding domain (NBD) leucine-rich repeat (LRR) family of proteins and regulates the transcription of MHC II by associating with the MHC enhanceosome.

In some embodiments, the target polynucleotide sequence of the present invention is a variant of CIITA. In some embodiments, the target polynucleotide sequence is a homolog of CIITA. In some embodiments, the target polynucleotide sequence is an ortholog of CIITA.

In some aspects, reduced or eliminated expression of CIITA reduces or eliminates expression of one or more of the following MHC class II are HLA-DP, HLA-DM, HLA-DOA, HLA-DOB, HLA-DQ, and HLA-DR.

In some embodiments, the hypoimmunogenic cells outlined herein comprise a genetic modification targeting the CIITA gene. In some embodiments, the genetic modification targeting the CIITA gene by the rare-cutting endonuclease comprises a Cas protein or a polynucleotide encoding a Cas protein, and at least one guide ribonucleic acid sequence for specifically targeting the CIITA gene. In some embodiments, the at least one guide ribonucleic acid sequence for specifically targeting the CIITA gene is selected from the group consisting of SEQ ID NOS:5184-36352 of Appendix 1 (Table 12 of WO2016183041) provided herewith. In some embodiments, the cell has a reduced ability to induce an immune response in a recipient subject.

Assays to test whether the CIITA gene has been inactivated are known and described herein. In one embodiment, the resulting genetic modification of the CIITA gene by PCR and the reduction of HLA-II expression can be assays by FACS analysis. In another embodiment, CIITA protein expression is detected using a Western blot of cells lysates probed with antibodies to the CIITA protein. In another embodiment, reverse transcriptase polymerase chain reactions (RT-PCR) are used to confirm the presence of the inactivating genetic modification.

2. B2M

In certain embodiments, the inventions disclosed herein modulate (e.g., reduce or eliminate) the expression of MHC-I genes by targeting and modulating (e.g., reducing or eliminating) expression of the accessory chain B2M. In some aspects, the modulation occurs using a CRISPR/Cas system. By modulating (e.g., reducing or deleting) expression of B2M, surface trafficking of MHC-I molecules is blocked and the cell rendered hypoimmunogenic. In some embodiments, the cell has a reduced ability to induce an immune response in a recipient subject.

In some embodiments, the target polynucleotide sequence of the present invention is a variant of B2M. In some embodiments, the target polynucleotide sequence is a homolog of B2M. In some embodiments, the target polynucleotide sequence is an ortholog of B2M.

In some aspects, decreased or eliminated expression of B2M reduces or eliminates expression of one or more of the following MHC I molecules—HLA-A, HLA-B, and HLA-C.

In some embodiments, the hypoimmunogenic cells outlined herein comprise a genetic modification targeting the B2M gene. In some embodiments, the genetic modification targeting the B2M gene by the rare-cutting endonuclease comprises a Cas protein or a polynucleotide encoding a Cas protein, and at least one guide ribonucleic acid sequence for specifically targeting the B2M gene. In some embodiments, the at least one guide ribonucleic acid sequence for specifically targeting the B2M gene is selected from the group consisting of SEQ ID NOS:81240-85644 of Appendix 2 (Table 15 of WO2016/183041) provided herewith.

Assays to test whether the B2M gene has been inactivated are known and described herein. In one embodiment, the resulting genetic modification of the B2M gene by PCR and the reduction of HLA-I expression can be assays by FACS analysis. In another embodiment, B2M protein expression is detected using a Western blot of cells lysates probed with antibodies to the B2M protein. In another embodiment, reverse transcriptase polymerase chain reactions (RT-PCR) are used to confirm the presence of the inactivating genetic modification.

3. NLRC5

In certain aspects, the inventions disclosed herein modulate (e.g., reduce or eliminate) the expression of MHC-I genes by targeting and modulating (e.g., reducing or eliminating) expression of the NLR family, CARD domain containing 5/NOD27/CLR16.1 (NLRC5). In some aspects, the modulation occurs using a CRISPR/Cas system. NLRC5 is a critical regulator of MHC-I-mediated immune responses and, similar to CIITA, NLRC5 is highly inducible by IFN-γ and can translocate into the nucleus. NLRC5 activates the promoters of MHC-I genes and induces the transcription of MHC-I as well as related genes involved in MHC-I antigen presentation.

In some embodiments, the target polynucleotide sequence of the present invention is a variant of NLRC5. In some embodiments, the target polynucleotide sequence is a homolog of NLRC5. In some embodiments, the target polynucleotide sequence is an ortholog of NLRC5.

In some aspects, decreased or eliminated expression of NLRC5 reduces or eliminates expression of one or more of the following MHC I molecules—HLA-A, HLA-B, and HLA-C.

In some embodiments, the hypoimmunogenic cells outlined herein comprise a genetic modification targeting the NLRC5 gene. In some embodiments, the genetic modification targeting the NLRC5 gene by the rare-cutting endonuclease comprises a Cas protein or a polynucleotide encoding a Cas protein, and at least one guide ribonucleic acid sequence for specifically targeting the NLRC5 gene. In some embodiments, the at least one guide ribonucleic acid sequence for specifically targeting the NLRC5 gene is selected from the group consisting of SEQ ID NOS:36353-81239 of Appendix 3 (Table 14 of WO2016183041) provided herewith. In some embodiments, the cell has a reduced ability to induce an immune response in a recipient subject.

Assays to test whether the NLRC5 gene has been inactivated are known and described herein. In one embodiment, the resulting genetic modification of the NLRC5 gene by PCR and the reduction of HLA-I expression can be assays by FACS analysis. In another embodiment, NLRC5 protein expression is detected using a Western blot of cells lysates probed with antibodies to the NLRC5 protein. In another embodiment, reverse transcriptase polymerase chain reactions (RT-PCR) are used to confirm the presence of the inactivating genetic modification.

4. HLA-A/B/C

In some embodiments, the hypoimmunogenic cells of the present invention do not express or exhibit reduced expression of one or more of HLA-A, HLA-B, and HLA-C. In some embodiments, the hypoimmunogenic cells of the present invention do not express or exhibit reduced expression of HLA-A, HLA-B, and HLA-C.

In some embodiments, the present disclosure provides a stem cell (e.g., hypoimmunogenic stem cell) or population thereof comprising a genome in which the HLA-A gene has been edited to delete a contiguous stretch of genomic DNA, thereby reducing or eliminating surface expression of MHC class I molecules in the cell or population thereof. The contiguous stretch of genomic DNA can be deleted by contacting the cell or population thereof with a Cas protein or a nucleic acid encoding the Cas protein and at least one ribonucleic acid or at least one pair of ribonucleic acids.

In certain embodiments, the present disclosure provides a method for altering a target HLA-A sequence in a cell comprising contacting the HLA-A sequence with a clustered regularly interspaced short palindromic repeats-associated (Cas) protein and at least one ribonucleic acid or at least one pair of ribonucleic acids, wherein the ribonucleic acids direct Cas protein to and hybridize to a target motif of the target HLA-A polynucleotide sequence, wherein the target HLA-A polynucleotide sequence is cleaved, and wherein the at least one ribonucleic acid or the at least one pair of ribonucleic acids.

In some embodiments, the present disclosure provides a stem cell (e.g., hypoimmunogenic stem cell) or population thereof comprising a genome in which the HLA-B gene has been edited to delete a contiguous stretch of genomic DNA, thereby reducing or eliminating surface expression of MHC class I molecules in the cell or population thereof. The contiguous stretch of genomic DNA can be deleted by contacting the cell or population thereof with a Cas protein or a nucleic acid encoding the Cas protein and at least one ribonucleic acid or at least one pair of ribonucleic acids.

In certain embodiments, the present disclosure provides a method for altering a target HLA-B sequence in a cell comprising contacting the HLA-B sequence with a clustered regularly interspaced short palindromic repeats-associated (Cas) protein and at least one ribonucleic acid or at least one pair of ribonucleic acids, wherein the ribonucleic acids direct Cas protein to and hybridize to a target motif of the target HLA-B polynucleotide sequence, wherein the target HLA-B polynucleotide sequence is cleaved, and wherein the at least one ribonucleic acid or the at least one pair of ribonucleic acids.

In some embodiments, the present disclosure provides a stem cell (e.g., hypoimmunogenic stem cell) or population thereof comprising a genome in which the HLA-C gene has been edited to delete a contiguous stretch of genomic DNA, thereby reducing or eliminating surface expression of MHC class I molecules in the cell or population thereof. The contiguous stretch of genomic DNA can be deleted by contacting the cell or population thereof with a Cas protein or a nucleic acid encoding the Cas protein and at least one ribonucleic acid or at least one pair of ribonucleic acids.

In certain embodiments, the present disclosure provides a method for altering a target HLA-C sequence in a cell comprising contacting the HLA-C sequence with a clustered regularly interspaced short palindromic repeats-associated (Cas) protein and at least one ribonucleic acid or at least one pair of ribonucleic acids, wherein the ribonucleic acids direct Cas protein to and hybridize to a target motif of the target HLA-C polynucleotide sequence, wherein the target HLA-C polynucleotide sequence is cleaved, and wherein the at least one ribonucleic acid or the at least one pair of ribonucleic acids.

5. CD47

In some aspects, the inventions disclosed herein modulate (e.g., increase) the expression of CD47. In some embodiments, the present disclosure provides a stem cell (e.g., hypoimmunogenic stem cell) or population thereof comprising a genome in which the stem cell genome has been modified to express CD47. In some embodiments, the present disclosure provides a method for altering a stem cell genome to express CD47. In certain embodiments, at least one ribonucleic acid or at least one pair of ribonucleic acids may be utilized to facilitate the insertion of CD47 into a stem cell line. In certain embodiments, the at least one ribonucleic acid or the at least one pair of ribonucleic acids is selected from the group consisting of SEQ ID NOS:200784-231885 of Appendix 4 (Table 29 of WO2016183041) provided herein.

D. Methods of Genetic Modifications

The present invention contemplates altering target polynucleotide sequences in any manner which is available to the skilled artisan. In some embodiments, the method includes utilizing a CRISPR/Cas system of the present invention. For instance, base-editing systems including, but not limited to, disabled Cas9 proteins can be utilized. Any CRISPR/Cas system that is capable of altering a target polynucleotide sequence (e.g., target gene) in a cell can be used. Such CRISPR/Cas systems can employ a variety of Cas proteins (Haft et al. PLoS Comput Biol. 2005; 1(6)e60). The molecular machinery of such Cas proteins that allows the CRISPR/Cas system to alter target polynucleotide sequences in cells include RNA binding proteins, endo- and exo-nucleases, helicases, and polymerases. In some embodiments, alternatives to CRISPR/Cas systems can be used to alter the target polynucleotide sequences in a cell including, but not limited to, site-specific recombinases (SSRs), zinc finger nucleases, TALE-nucleases, meganucleases (also known as homing endonucleases), and the like.

CRISPR systems can be divided into two main classes, Class 1 and Class 2, which are further classified into different types and sub-types. The classification of the CRISPR systems is based on the effector Cas proteins that are capable of cleaving specific nucleic acids. In Class 1 CRISPR systems the effector module consists of a multi-protein complex, whereas Class 2 systems only use one effector protein. Class 1 CRISPR includes Types I, III, and IV and Class 2 CRISPR includes Types II, V, and VI. While any of these types of CRISPR systems may be used in accordance with the present invention, there are three types of CRISPR systems which incorporate RNAs and Cas proteins that are preferred for use in accordance with the present invention: Types I (exemplified by Cas3), II (exemplified by Cas9), and III (exemplified by Cas10). The Type II CRISPR is one of the most well-characterized systems. In some embodiments, the CRISPR/Cas system is a CRISPR type I system. In some embodiments, the CRISPR/Cas system is a CRISPR type II system. In some embodiments, the CRISPR/Cas system is a CRISPR type V system. In some embodiments, the CRISPR/Cas system is a CRISPR type VI system.

The CRISPR/Cas systems of the present technology can be used to alter any target polynucleotide sequence in a cell. Those skilled in the art will readily appreciate that desirable target polynucleotide sequences to be altered in any particular cell may correspond to any genomic sequence for which expression of the genomic sequence is associated with a disorder or otherwise facilitates entry of a pathogen into the cell. For example, a desirable target polynucleotide sequence to alter in a cell may be a polynucleotide sequence corresponding to a genomic sequence which contains a disease associated single polynucleotide polymorphism. In such example, the CRISPR/Cas systems of the present technology can be used to correct the disease associated SNP in a cell by replacing it with a wild-type allele. As another example, a polynucleotide sequence of a target gene which is responsible for entry or proliferation of a pathogen into a cell may be a suitable target for deletion or insertion to disrupt the function of the target gene to prevent the pathogen from entering the cell or proliferating inside the cell.

In some embodiments, the target polynucleotide sequence is a genomic sequence. In some embodiments, the target polynucleotide sequence is a human genomic sequence. In some embodiments, the target polynucleotide sequence is a mammalian genomic sequence. In some embodiments, the target polynucleotide sequence is a vertebrate genomic sequence.

In some embodiments, a CRISPR/Cas system of the present invention includes a Cas protein and at least one to two ribonucleic acids (e.g., guide RNAs or guide RNA target sequences) that are capable of directing the Cas protein to and hybridizing to a target motif of a target polynucleotide sequence. As used herein, “protein” and “polypeptide” are used interchangeably to refer to a series of amino acid residues joined by peptide bonds (i.e., a polymer of amino acids) and include modified amino acids (e.g., phosphorylated, glycated, glycosylated, etc.) and amino acid analogs. Exemplary polypeptides or proteins include gene products, naturally occurring proteins, homologs, paralogs, fragments and other equivalents, variants, and analogs of the above.

In some embodiments, a Cas protein comprises one or more amino acid substitutions or modifications. In some embodiments, the one or more amino acid substitutions comprises a conservative amino acid substitution. In some instances, substitutions and/or modifications can prevent or reduce proteolytic degradation and/or extend the half-life of the polypeptide in a cell. In some embodiments, the Cas protein can comprise a peptide bond replacement (e.g., urea, thiourea, carbamate, sulfonyl urea, etc.). In some embodiments, the Cas protein can comprise a naturally occurring amino acid. In some embodiments, the Cas protein can comprise an alternative amino acid (e.g., D-amino acids, beta-amino acids, homocysteine, phosphoserine, etc.). In some embodiments, a Cas protein can comprise a modification to include a moiety (e.g., PEGylation, glycosylation, lipidation, acetylation, end-capping, etc.).

In some embodiments, a Cas protein comprises a core Cas protein. Exemplary Cas core proteins include, but are not limited to Cas1, Cas1B, Cas2, Cas3, Cas4, Cas5, Cas5d, Cas5t, Cas5h, Cas5a, Cas6, Cas7, Cas8, Cas9 (also known as Csn1 or Csx12), Cas10, Cas11, Cas12, Cas13, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Csd1, Csd2, Cst1, Cst2, Csh1, Csh2, Csa1, Csa2, Csa3, Csa4, Csa5, homologues thereof, or modified versions thereof. In some embodiments, a Cas protein comprises a Cas protein of an E. coli subtype (also known as CASS2). Exemplary Cas proteins of the E. Coli subtype include, but are not limited to Cse1, Cse2, Cse3, Cse4, and Cas5e. In some embodiments, a Cas protein comprises a Cas protein of the Ypest subtype (also known as CASS3). Exemplary Cas proteins of the Ypest subtype include, but are not limited to Csy1, Csy2, Csy3, and Csy4. In some embodiments, a Cas protein comprises a Cas protein of the Nmeni subtype (also known as CASS4). Exemplary Cas proteins of the Nmeni subtype include, but are not limited to Csn1 and Csn2. In some embodiments, a Cas protein comprises a Cas protein of the Dvulg subtype (also known as CASS1). Exemplary Cas proteins of the Dvulg subtype include Csd1, Csd2, and Cas5d. In some embodiments, a Cas protein comprises a Cas protein of the Tneap subtype (also known as CASS7). Exemplary Cas proteins of the Tneap subtype include, but are not limited to, Cst1, Cst2, Cas5t. In some embodiments, a Cas protein comprises a Cas protein of the Hmari subtype. Exemplary Cas proteins of the Hmari subtype include, but are not limited to Csh1, Csh2, and Cas5h. In some embodiments, a Cas protein comprises a Cas protein of the Apern subtype (also known as CASS5). Exemplary Cas proteins of the Apern subtype include, but are not limited to Csa1, Csa2, Csa3, Csa4, Csa5, and Cas5a. In some embodiments, a Cas protein comprises a Cas protein of the Mtube subtype (also known as CASS6). Exemplary Cas proteins of the Mtube subtype include, but are not limited to Csm1, Csm2, Csm3, Csm4, and Csm5. In some embodiments, a Cas protein comprises a RAMP module Cas protein. Exemplary RAMP module Cas proteins include, but are not limited to, Cmr1, Cmr2, Cmr3, Cmr4, Cmr5, and Cmr6. See, e.g., Klompe et al., Nature 571, 219-225 (2019); Strecker et al., Science 365, 48-53 (2019).

In some embodiments, a Cas protein comprises any one of the Cas proteins described herein or a functional portion thereof. As used herein, “functional portion” refers to a portion of a peptide which retains its ability to complex with at least one ribonucleic acid (e.g., guide RNA (gRNA)) and cleave a target polynucleotide sequence. In some embodiments, the functional portion comprises a combination of operably linked Cas9 protein functional domains selected from the group consisting of a DNA binding domain, at least one RNA binding domain, a helicase domain, and an endonuclease domain. In some embodiments, the functional portion comprises a combination of operably linked Cas12a (also known as Cpf1) protein functional domains selected from the group consisting of a DNA binding domain, at least one RNA binding domain, a helicase domain, and an endonuclease domain. In some embodiments, the functional domains form a complex. In some embodiments, a functional portion of the Cas9 protein comprises a functional portion of a RuvC-like domain. In some embodiments, a functional portion of the Cas9 protein comprises a functional portion of the HNH nuclease domain. In some embodiments, a functional portion of the Cas12a protein comprises a functional portion of a RuvC-like domain.

In some embodiments, exogenous Cas protein can be introduced into the cell in polypeptide form. In certain embodiments, Cas proteins can be conjugated to or fused to a cell-penetrating polypeptide or cell-penetrating peptide. As used herein, “cell-penetrating polypeptide” and “cell-penetrating peptide” refers to a polypeptide or peptide, respectively, which facilitates the uptake of molecule into a cell. The cell-penetrating polypeptides can contain a detectable label.

In certain embodiments, Cas proteins can be conjugated to or fused to a charged protein (e.g., that carries a positive, negative or overall neutral electric charge). Such linkage may be covalent. In some embodiments, the Cas protein can be fused to a superpositively charged GFP to significantly increase the ability of the Cas protein to penetrate a cell (Cronican et al. ACS Chem Biol. 2010; 5(8):747-52). In certain embodiments, the Cas protein can be fused to a protein transduction domain (PTD) to facilitate its entry into a cell. Exemplary PTDs include Tat, oligoarginine, and penetratin. In some embodiments, the Cas9 protein comprises a Cas9 polypeptide fused to a cell-penetrating peptide. In some embodiments, the Cas9 protein comprises a Cas9 polypeptide fused to a PTD. In some embodiments, the Cas9 protein comprises a Cas9 polypeptide fused to a tat domain. In some embodiments, the Cas9 protein comprises a Cas9 polypeptide fused to an oligoarginine domain. In some embodiments, the Cas9 protein comprises a Cas9 polypeptide fused to a penetratin domain. In some embodiments, the Cas9 protein comprises a Cas9 polypeptide fused to a superpositively charged GFP. In some embodiments, the Cas12a protein comprises a Cas12a polypeptide fused to a cell-penetrating peptide. In some embodiments, the Cas12a protein comprises a Cas12a polypeptide fused to a PTD. In some embodiments, the Cas12a protein comprises a Cas12a polypeptide fused to a tat domain. In some embodiments, the Cas12a protein comprises a Cas12a polypeptide fused to an oligoarginine domain. In some embodiments, the Cas12a protein comprises a Cas12a polypeptide fused to a penetratin domain. In some embodiments, the Cas12a protein comprises a Cas12a polypeptide fused to a superpositively charged GFP.

In some embodiments, the Cas protein can be introduced into a cell containing the target polynucleotide sequence in the form of a nucleic acid encoding the Cas protein. The process of introducing the nucleic acids into cells can be achieved by any suitable technique. Suitable techniques include calcium phosphate or lipid-mediated transfection, electroporation, and transduction or infection using a viral vector. In some embodiments, the nucleic acid comprises DNA. In some embodiments, the nucleic acid comprises a modified DNA, as described herein. In some embodiments, the nucleic acid comprises mRNA. In some embodiments, the nucleic acid comprises a modified mRNA, as described herein (e.g., a synthetic, modified mRNA).

Cas9 mediates genome editing at sites complementary to a 20-nucleotide sequence (e.g., protospacer) in a bound guide RNA. In addition, target sites must include a protospacer adjacent motif (PAM) at the 3′ end adjacent to the 20-nucleotide target site; for Streptococcus pyogenes Cas9, the PAM sequence is NGG. The methods of the present invention contemplate the use of any ribonucleic acid that is capable of directing a Cas protein to and hybridizing to a target motif of a target polynucleotide sequence. In some embodiments, at least one of the ribonucleic acids comprises tracrRNA. In some embodiments, at least one of the ribonucleic acids comprises CRISPR RNA (crRNA). In some embodiments, a single ribonucleic acid comprises a guide RNA that directs the Cas protein to and hybridizes to a target motif of the target polynucleotide sequence in a cell. In some embodiments, at least one of the ribonucleic acids comprises a segment that directs the Cas protein to and hybridizes to a target motif of the target polynucleotide sequence in a cell. In some embodiments, both of the one to two ribonucleic acids comprise a segment that directs the Cas protein to and hybridizes to a target motif of the target, polynucleotide sequence in a cell, The ribonucleic acids of the present invention can be selected to hybridize to a variety of different target motifs, depending on the particular CRISPR/Cas system employed, and the sequence of the target polynucleotide, as will be appreciated by those skilled in the art. The one to two ribonucleic acids can also be selected to minimize hybridization with nucleic acid sequences other than the target polynucleotide sequence. In some embodiments, the one to two ribonucleic acids, hybridize to a target motif that contains at least two mismatches when compared with all other genomic nucleotide sequences in the cell. gRNA molecules generally include a targeting domain and a tracr. In some embodiments the targeting domain and tracr are disposed on a single polynucleotide. In some embodiments, a gRNA molecule consists of a single contiguous polynucleotide molecule, referred to herein as a “single guide RNA” or “sgRNA” and the like. In other embodiments, a gRNA molecule consists of a plurality, usually two, polynucleotide molecules, which are themselves capable of association, usually through hybridization, referred to herein as a “dual guide RNA” or “dgRNA, “two-part guide RNA”, and the like.

It has been suggested that complementarity between guide RNA and target DNA is required in the 7-12 base pairs adjacent to the PAM end of the target site (3′ end of the guide RNA) and mismatches are tolerated at the non-PAM end (5′ end of the guide RNA). In some embodiments, there are at least 1 mismatch between the DNA-binding segment of the guide RNA and the target DNA. In some embodiments, there are at least 2 mismatches between the DNA-binding segment of the guide RNA and the target DNA. In some embodiments, there are at least 3 mismatches between the DNA-binding segment of the guide RNA and the target DNA. In some embodiments, there are at least 4 mismatches between the DNA-binding segment of the guide RNA and the target DNA. In some embodiments, there are at least 5 mismatches between the DNA-binding segment of the guide RNA and the target DNA. In some embodiments, the Cas protein is complexed with one to two ribonucleic acid sequences (e.g., gRNAs). In some embodiments, the Cas protein is complexed with two ribonucleic acid sequences. In some embodiments, the Cas protein is complexed with one ribonucleic acid sequence. In some embodiments, the Cas protein is encoded by a modified nucleic acid, as described herein (e.g., a synthetic, modified mRNA).

The methods of the present invention contemplate the use of any ribonucleic acid sequence that is capable of directing a Cas protein to and hybridizing to a target motif of a target polynucleotide sequence. In some embodiments, a single guide RNA is used. In some embodiments, dual guide RNAs are used. In some embodiments, at least one of the ribonucleic acid sequence comprises tracrRNA. In some embodiments, at least one of the ribonucleic acid sequence comprises CRISPR RNA (crRNA). In some embodiments, a single ribonucleic acid comprises a guide RNA that directs the Cas protein to and hybridizes to a target motif of the target polynucleotide sequence in a cell. In some embodiments, the ribonucleic acid sequence is a guide RNA comprising a crRNA and tracrRNA that directs the Cas protein to and hybridizes to a target motif of the target polynucleotide sequence in a cell.

A ribonucleic acid sequence of a guide RNA that is specific to the target sequence (e.g., protospacer) can be designed by replacing thymines with uracils for the crRNA and adding a tracrRNA sequence. A guide RNA does not include the PAM sequence which serves as a binding signal for the Cas protein. From the target sequence, a guide RNA can be designed comprising a crRNA that is complementary to the target sequence. Guide RNA sequences are also designed with minimal off-targeting binding and cleavage. In some instances, off-target prediction methods and algorithms, including those described in Hsu et al., Nature Biotechnology, 2013, 31, 827-832, are employed to evaluate guide RNA target sequences. Provided herein are guide RNAs comprising an RNA sequence corresponding to gRNA targets disclosed in any of the tables and Figures.

The ribonucleic acids of the present invention can be selected to hybridize to a variety of different target motifs, depending on the particular CRISPR/Cas system employed, and the sequence of the target polynucleotide, as will be appreciated by those skilled in the art. The one to two ribonucleic acids can also be selected to minimize hybridization with nucleic acid sequences other than the target polynucleotide sequence. In some embodiments, the one to two ribonucleic acids hybridize to a target motif that contains at least two mismatches when compared with all other genomic nucleotide sequences in the cell. In some embodiments, the one to two ribonucleic acids hybridize to a target motif that contains at least one mismatch when compared with all other genomic nucleotide sequences in the cell. In some embodiments, the one to two ribonucleic acids are designed to hybridize to a target motif immediately adjacent to a deoxyribonucleic acid motif recognized by the Cas protein. In some embodiments, each of the one to two ribonucleic acids are designed to hybridize to target motifs immediately adjacent to deoxyribonucleic acid motifs recognized by the Cas protein which flank a mutant allele located between the target motifs.

In some embodiments, each of the one to two ribonucleic acids comprises guide RNAs that directs the Cas protein to and hybridizes to a target motif of the target polynucleotide sequence in a cell.

In some embodiments, one or two ribonucleic acids (e.g., guide RNAs) are complementary to and/or hybridize to sequences on the same strand of a target polynucleotide sequence. In some embodiments, one or two ribonucleic acids (e.g., guide RNAs) are complementary to and/or hybridize to sequences on the opposite strands of a target polynucleotide sequence. In some embodiments, the one or two ribonucleic acids (e.g., guide RNAs) are not complementary to and/or do not hybridize to sequences on the opposite strands of a target polynucleotide sequence. In some embodiments, the one or two ribonucleic acids (e.g., guide RNAs) are complementary to and/or hybridize to overlapping target motifs of a target polynucleotide sequence. In some embodiments, the one or two ribonucleic acids (e.g., guide RNAs) are complementary to and/or hybridize to offset target motifs of a target polynucleotide sequence.

In some embodiments, a target nucleic acid sequence for genome editing may have a specific sequence on its 3′ end, named the protospacer adjacent motif or protospacer associated motif (PAM). The PAM is present in the targeted nucleic acid sequence but not in the crRNA that is produced to target it. In some embodiments, the proto-spacer adjacent motif (PAM) may correspond to 2 to 5 nucleotides starting immediately or in the vicinity of the proto-spacer at the leader distal end. The sequence and the location of the PAM vary among the different systems. Non-limiting examples of PAM motif include NNAGAA (SEQ ID NO:98), NAG, NGG, NGGNG (SEQ ID NO:99), AWG, CC, CC, CCN, TCN, and TTC. Different Type II CRISPR systems have differing PAM requirements. For example, the S. pyogenes system requires an NGG sequence, where N can be any nucleotides. In some cases, a Cas9 protein can be engineered to recognize specific PAM motifs or to recognize a non-natural PAM motif. In some instances, the selected target sequence may comprise a smaller or a larger PAM motif with any combinations of amino acids. In some embodiments, a selected target sequence comprise a PAM motif which comprises at least 3, preferably, 4, more preferably 5 nucleotides recognized by a Cas9 protein or a variant thereof.

In some embodiments, nucleic acids encoding Cas protein and nucleic acids encoding the at least one to two ribonucleic acids are introduced into a cell via viral transduction (e.g., lentiviral transduction). In some embodiments, the Cas protein is complexed with 1-2 ribonucleic acids. In some embodiments, the Cas protein is complexed with two ribonucleic acids. In some embodiments, the Cas protein is complexed with one ribonucleic acid. In some embodiments, the Cas protein is encoded by a modified nucleic acid, as described herein (e.g., a synthetic, modified mRNA). In some embodiments, a guide RNA comprises a peptide nucleic acid (PNA) or a locked nucleic acid (LNA).

Exemplary gRNA sequences useful for CRISPR/Cas-based targeting of genes described herein are provided in Table 1. The sequences can be found in WO2016/183041 filed May 9, 2016, the disclosure including the Tables, Appendices, and Sequence Listing is incorporated herein by reference in its entirety.

Additional disclosures of CRISPR/Cas system are provided in U.S. Pat. Nos. 8,697,359; 8,993,233; 8,795,965; 8,771,945; 8,889,356; 8,865,406; 8,999,641; 8,945,839; 8,932,814; 8,871,445; 8,906,616; and U.S. Pat. No. 8,895,308, Yan et al., Science 363, 88-91 (2019), Moon et al. Experimental & Molecular Medicine (2019) 51:130, and Tachibana, Science (2019; www.sciencemag.org/features/2019/09/beyond-crispr-what-s-current-and-upcoming-genome-editing), the disclosures including the figures, figure legends, and description of methods are incorporated herein by reference in their entirety. Exemplary gRNA sequences useful for CRISPR/Cas-based targeting of genes described herein are provided in Table 5. The sequences can be found in WO2016183041 filed May 9, 2016, the disclosure including the Tables, Appendices, and Sequence Listing is incorporated herein by reference in its entirety.

TABLE 5
Exemplary gRNA sequences useful for targeting genes

| Gene Name | SEQ ID NO: | WO2016183041 |
|---|---|---|
| HLA-A | SEQ ID NOs: 2-1418 | Table 8, Appendix 1 |
| HLA-B | SEQ ID NOs: 1419-3277 | Table 9, Appendix 2 |
| HLA-C | SEQ ID NOs: 3278-5183 | Table 10, Appendix 3 |
| RFX-ANK | SEQ ID NOs: 95636-102318 | Table 11, Appendix 4 |
| NFY-A | SEQ ID NOs: 102319-121796 | Table 13, Appendix 6 |
| RFX5 | SEQ ID NOs: 85645-90115 | Table 16, Appendix 9 |
| RFX-AP | SEQ ID NOs: 90116-95635 | Table 17, Appendix 10 |
| NFY-B | SEQ ID NOs: 121797-135112 | Table 20, Appendix 13 |
| NFY-C | SEQ ID NOs: 135113-176601 | Table 22, Appendix 15 |
| IRF1 | SEQ ID NOs: 176602-182813 | Table 23, Appendix 16 |
| TAP1 | SEQ ID NOs: 182814-188371 | Table 24, Appendix 17 |
| CIITA | SEQ ID NOs: 5184-36352 | Table 12, Appendix 5 |
| B2M | SEQ ID NOs: 81240-85644 | Table 15, Appendix 8 |
| NLRC5 | SEQ ID NOs: 36353-81239 | Table 14, Appendix 7 |
| CD47 | SEQ ID NOs: 200784-231885 | Table 29, Appendix 22 |
| HLA-E | SEQ ID NOs: 189859-193183 | Table 19, Appendix 12 |
| HLA-F | SEQ ID NOs: 688808-699754 | Table 45, Appendix 38 |
| HLA-G | SEQ ID NOs: 188372-189858 | Table 18, Appendix 11 |
| PD-L1 | SEQ ID NOs: 193184-200783 | Table 21, Appendix 14 |

In some embodiments, the cells of the invention are made using Transcription Activator-Like Effector Nucleases (TALEN) methodologies.

By a “TALE-nuclease” (TALEN) is intended a fusion protein consisting of a nucleic acid-binding domain typically derived from a Transcription Activator Like Effector (TALE) and one nuclease catalytic domain to cleave a nucleic acid target sequence. The catalytic domain is preferably a nuclease domain and more preferably a domain having endonuclease activity, like for instance I-TevI, ColE7, NucA and Fok-I. TALE-nucleases are specific reagents because they need to bind DNA by pairs under obligatory heterodimeric form to obtain dimerization of the cleavage domain such as Fok-1. Left and right heterodimer members each recognizes a different nucleic sequences of about 14 to 20 bp, together spanning target sequences of 30 to 50 bp overall specificity. To induce site-specific mutation, two individual TALEN arms, separated by a 14-20 base pair spacer region, bring FokI monomers in close proximity to dimerize and produce a targeted double-strand break. In a particular embodiment, the TALE domain can be fused to a meganuclease like for instance I-CreI and I-OnuI or functional variant thereof. In some embodiments, the TALE-nucleases are heterodimeric nucleases. In some embodiments, the TALE-nucleases are monomeric TALE-nucleases. A monomeric TALE-Nuclease is a TALE-nuclease that does not require dimerization for specific recognition and cleavage, such as the fusions of engineered TAL repeats with the catalytic domain of I-TevI described in WO2012138927. Transcription Activator like Effector (TALE) are proteins from the bacterial species Xanthomonas comprise a plurality of repeated sequences, each repeat comprising di-residues in position 12 and 13 (RVD) that are specific to each nucleotide base of the nucleic acid targeted sequence. Binding domains with similar modular base-per-base nucleic acid binding properties (MBBBD) can also be derived from new modular proteins recently discovered by the applicant in a different bacterial species. The new modular proteins have the advantage of displaying more sequence variability than TAL repeats. Preferably, RVDs associated with recognition of the different nucleotides are HD for recognizing C, NG for recognizing T, NI for recognizing A, NN for recognizing G or A, NS for recognizing A, C, G or T, HG for recognizing T, IG for recognizing T, NK for recognizing G, HA for recognizing C, ND for recognizing C, HI for recognizing C, HN for recognizing G, NA for recognizing G, SN for recognizing G or A and YG for recognizing T, TL for recognizing A, VT for recognizing A or G and SW for recognizing A. In another embodiment, critical amino acids 12 and 13 can be mutated towards other amino acid residues in order to modulate their specificity towards nucleotides A, T, C and G and in particular to enhance this specificity. TALEN kits are sold commercially.

In some embodiments, the cells are manipulated using zinc finger nuclease (ZFN). A “zinc finger binding protein” is a protein or polypeptide that binds DNA, RNA and/or protein, preferably in a sequence-specific manner, as a result of stabilization of protein structure through coordination of a zinc ion. The term zinc finger binding protein is often abbreviated as zinc finger protein or ZFP. The individual DNA binding domains are typically referred to as “fingers.” A ZFP has least one finger, typically two fingers, three fingers, or six fingers. Each finger binds from two to four base pairs of DNA, typically three or four base pairs of DNA. A ZFP binds to a nucleic acid sequence called a target site or target segment. Each finger typically comprises an approximately 30 amino acid, zinc-chelating, DNA-binding subdomain. Studies have demonstrated that a single zinc finger of this class consists of an alpha helix containing the two invariant histidine residues co-ordinated with zinc along with the two cysteine residues of a single beta turn (see, e.g., Berg & Shi, Science 271:1081-1085 (1996)).

In some embodiments, the cells of the invention are made using a homing endonuclease. Such homing endonucleases are well-known to the art (Stoddard 2005). Homing endonucleases recognize a DNA target sequence and generate a single- or double-strand break. Homing endonucleases are highly specific, recognizing DNA target sites ranging from 12 to 45 base pairs (bp) in length, usually ranging from 14 to 40 bp in length. The homing endonuclease according to the invention may for example correspond to a LAGLIDADG endonuclease, to a HNH endonuclease, or to a GIY-YIG endonuclease. Preferred homing endonuclease according to the present invention can be an I-CreI variant.

In some embodiments, the cells of the invention are made using a meganuclease. Meganucleases are by definition sequence-specific endonucleases recognizing large sequences (Chevalier, B. S. and B. L. Stoddard, Nucleic Acids Res., 2001, 29, 3757-3774). They can cleave unique sites in living cells, thereby enhancing gene targeting by 1000-fold or more in the vicinity of the cleavage site (Puchta et al., Nucleic Acids Res., 1993, 21, 5034-5040; Rouet et al., Mol. Cell. Biol., 1994, 14, 8096-8106; Choulika et al., Mol. Cell. Biol., 1995, 15, 1968-1973; Puchta et al., Proc. Natl. Acad. Sci. USA, 1996, 93, 5055-5060; Sargent et al., Mol. Cell. Biol., 1997, 17, 267-77; Donoho et al., Mol. Cell. Biol, 1998, 18, 4070-4078; Elliott et al., Mol. Cell. Biol., 1998, 18, 93-101; Cohen-Tannoudji et al., Mol. Cell. Biol., 1998, 18, 1444-1448).

In some embodiments, the cells of the invention are made using RNA silencing or RNA interference (RNAi) to knockdown (e.g., decrease, eliminate, or inhibit) the expression of a polypeptide such as a hypoimmunity factor. Useful RNAi methods include those that utilize synthetic RNAi molecules, short interfering RNAs (siRNAs), PIWI-interacting NRAs (piRNAs), short hairpin RNAs (shRNAs), microRNAs (miRNAs), and other transient knockdown methods recognized by those skilled in the art. Reagents for RNAi including sequence specific shRNAs, siRNA, miRNAs and the like are commercially available. Reagents for RNAi including sequence specific shRNAs, siRNA, miRNAs and the like are commercially available. For instance, CIITA can be knocked down in a pluripotent stem cell by introducing a CIITA siRNA or transducing a CIITA shRNA-expressing virus into the cell. In some embodiments, RNA interference is employed to reduce or inhibit the expression of at least one selected from the group consisting of CIITA, B2M, ABO, RHD and FUT1.

In some embodiments, the rare-cutting endonuclease is introduced into a cell containing the target polynucleotide sequence in the form of a nucleic acid encoding a rare-cutting endonuclease. The process of introducing the nucleic acids into cells can be achieved by any suitable technique. Suitable techniques include calcium phosphate or lipid-mediated transfection, electroporation, and transduction or infection using a viral vector. In some embodiments, the nucleic acid comprises DNA. In some embodiments, the nucleic acid comprises a modified DNA, as described herein. In some embodiments, the nucleic acid comprises mRNA. In some embodiments, the nucleic acid comprises a modified mRNA, as described herein (e.g., a synthetic, modified mRNA).

The process of introducing the nucleic acids into cells can be achieved by any suitable technique. The gene editing system used herein, such as CRISPR, TALEN, and ZFN systems, can be delivered into a cell through any suitable technique, including but not limited to calcium phosphate or lipid-mediated transfection, electroporation, and transduction or infection using a viral vector. In some embodiment, the gene editing system of the present invention is delivered to a cell via electroporation. In some embodiments, the electroporation system is a flow electroporation system. An example of a suitable flow electroporation system suitable for use with some embodiments of the present invention is the commercially-available MaxCyte STX system. There are several alternative commercially-available electroporation instruments which may be suitable for use with the present invention, such as the AgilePulse system or ECM 830 available from BTX-Harvard Apparatus, Cellaxess Elektra (Cellectricon), Nucleofector (Lonza/Amaxa), GenePulser MXcell (BIORAD), iPorator-96 (Primax) or siPORTer96 (Ambion). In some embodiments, the electroporation system forms a closed, sterile system for a cell. In some embodiments, the electroporation system is a pulsed electroporation system as described herein, and forms a closed, sterile system for a cell.

E. Overexpression of Tolerogenic Factors

For all of the technologies described herein, well-known recombinant techniques can be used to generate recombinant nucleic acids. In certain aspects, overexpression of tolerogenic factors is accomplished utilizing regulatory sequences within an expression construct. In certain embodiments, the recombinant nucleic acids encoding a tolerogenic factor may be operably linked to one or more regulatory nucleotide sequences in an expression construct. Regulatory nucleotide sequences will generally be appropriate for the host cell and recipient subject to be treated. Numerous types of appropriate expression vectors and suitable regulatory sequences are known in the art for a variety of host cells and are compatible with the technologies disclosed herein. Typically, the one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are also contemplated. The promoters may be either naturally occurring promoters, or hybrid promoters that combine elements of more than one promoter. An expression construct may be present in a cell on an episome, such as a plasmid, or the expression construct may be inserted into a chromosome. In a specific embodiment, the expression vector includes a selectable marker gene to allow the selection of transformed host cells. Certain embodiments include an expression vector comprising a nucleotide sequence encoding a variant polypeptide operably linked to at least one regulatory sequence. Regulatory sequences for use herein include promoters, enhancers, and other expression control elements. In certain embodiments, an expression vector is designed for the choice of the host cell to be transformed, the particular variant polypeptide desired to be expressed, the vector's copy number, the ability to control that copy number, and/or the expression of any other protein encoded by the vector, such as antibiotic markers.

Examples of suitable mammalian promoters include, for example, promoters from the following genes: elongation factor 1 alpha (EF1α) promoter, ubiquitin/S27a promoter of the hamster (WO 97/15664), Simian vacuolating virus 40 (SV40) early promoter, adenovirus major late promoter, mouse metallothionein-I promoter, the long terminal repeat region of Rous Sarcoma Virus (RSV), mouse mammary tumor virus promoter (MMTV), Moloney murine leukemia virus Long Terminal repeat region, and the early promoter of human Cytomegalovirus (CMV). Examples of other heterologous mammalian promoters are the actin, immunoglobulin or heat shock promoter(s). In additional embodiments, promoters for use in mammalian host cells can be obtained from the genomes of viruses such as polyoma virus, fowlpox virus (UK 2,211,504 published 5 Jul. 1989), bovine papilloma virus, avian sarcoma virus, cytomegalovirus, a retrovirus, hepatitis-B virus and Simian Virus 40 (SV40). In further embodiments, heterologous mammalian promoters are used. Examples include the actin promoter, an immunoglobulin promoter, and heat-shock promoters. The early and late promoters of SV40 are conveniently obtained as an SV40 restriction fragment which also contains the SV40 viral origin of replication (Fiers et al, Nature 273: 113-120 (1978)). The immediate early promoter of the human cytomegalovirus is conveniently obtained as a HindIII restriction enzyme fragment (Greenaway et al, Gene 18: 355-360 (1982)). The foregoing references are incorporated by reference in their entirety.

The process of introducing the polynucleotides described herein into cells can be achieved by any suitable technique. Suitable techniques include calcium phosphate or lipid-mediated transfection, electroporation, and transduction or infection using a viral vector. In some embodiments, the polynucleotides are introduced into a cell via viral transduction (e.g., lentiviral transduction).

Once altered, the presence of expression of any of the molecule described herein can be assayed using known techniques, such as Western blots, ELISA assays, FACS assays, and the like.

F. Assays for Hypoimmunogenicity Phenotypes and Retention of Pluripotency

Once the hypoimmunogenic cells have been generated, they may be assayed for their hypoimmunogenicity and/or retention of pluripotency as is described in WO2016183041 and WO2018132783.

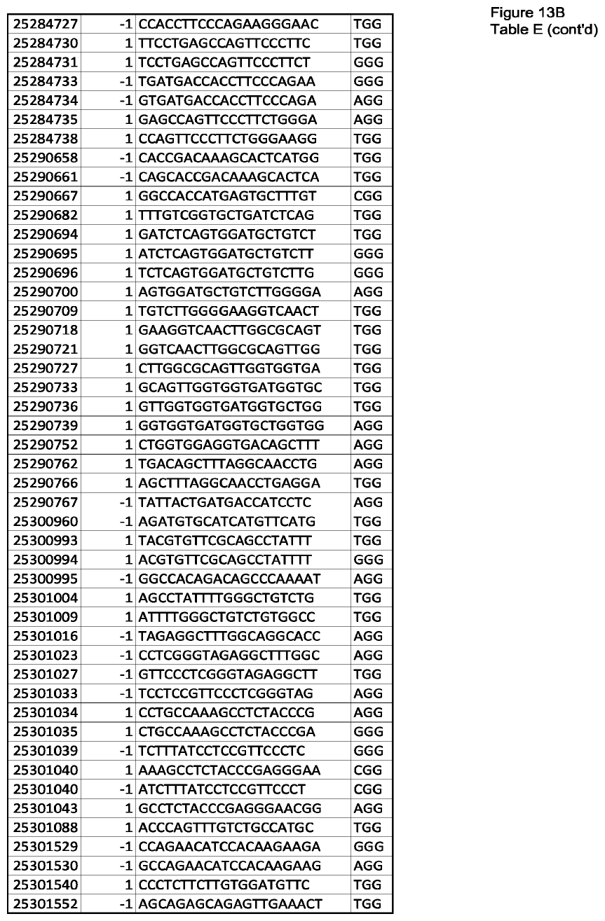

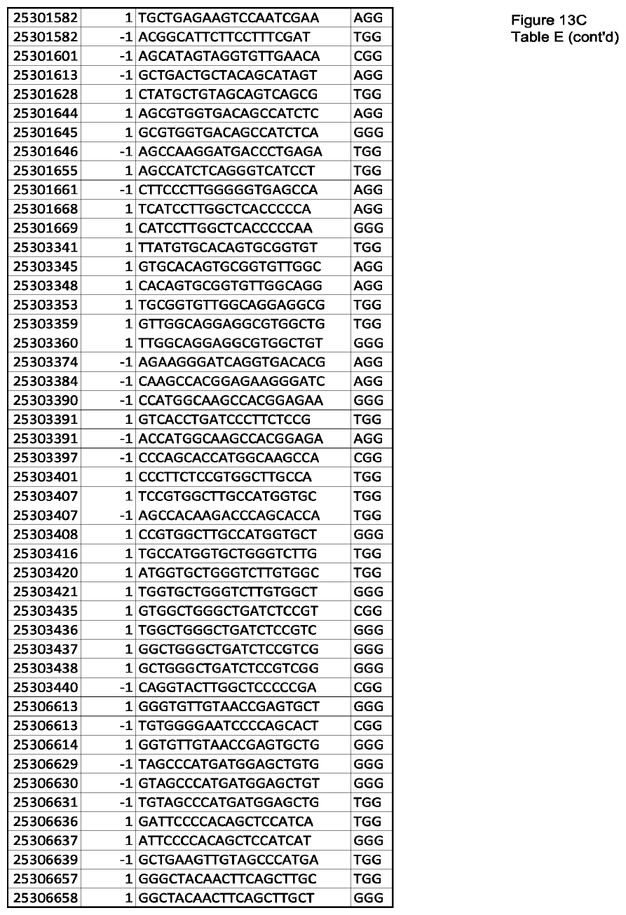

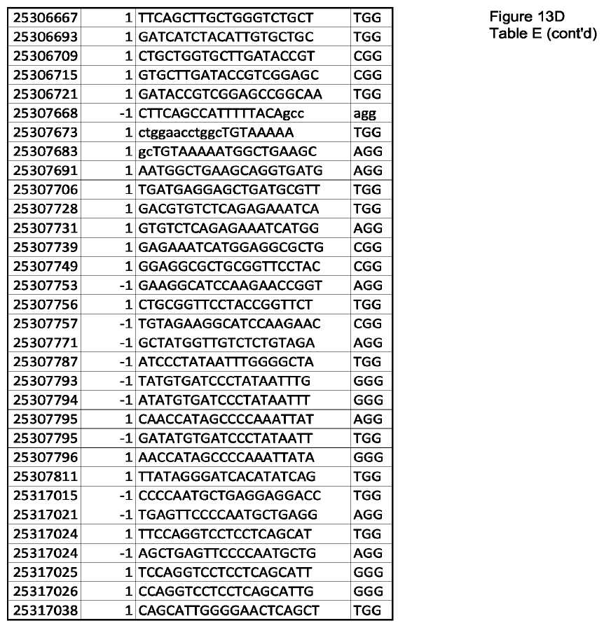

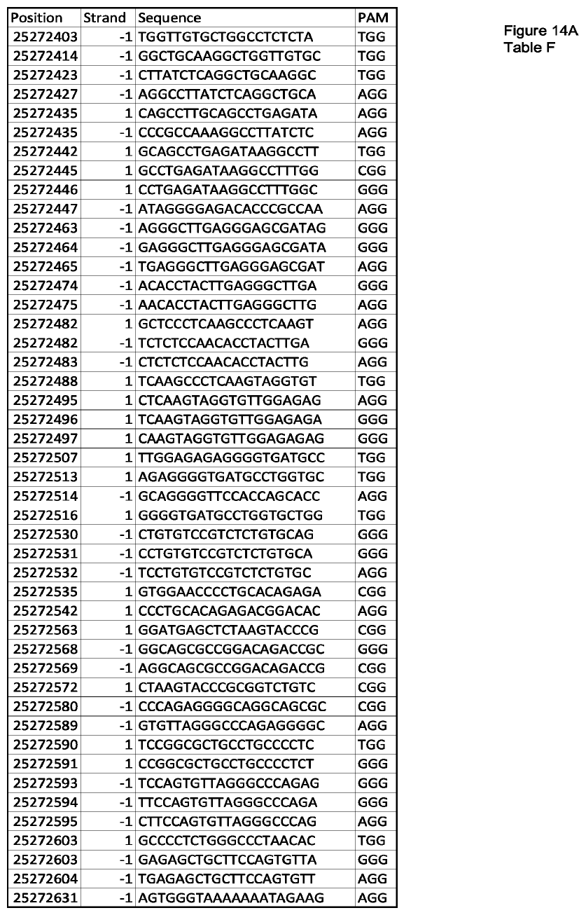

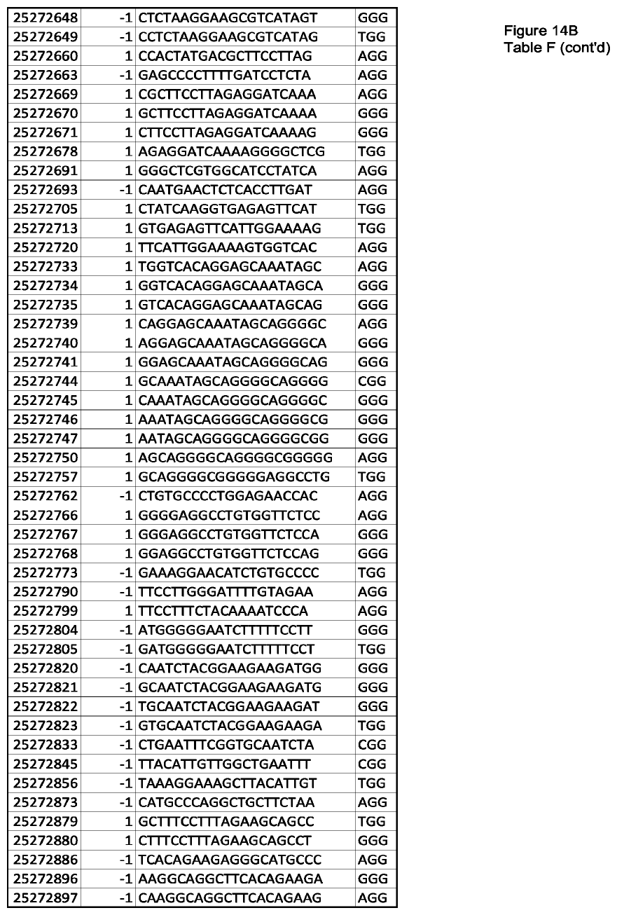

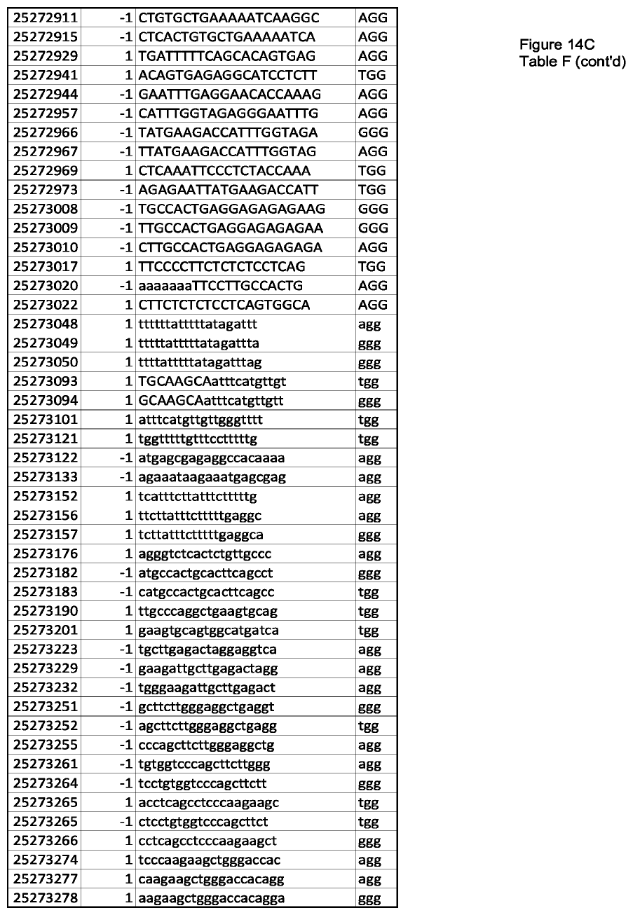

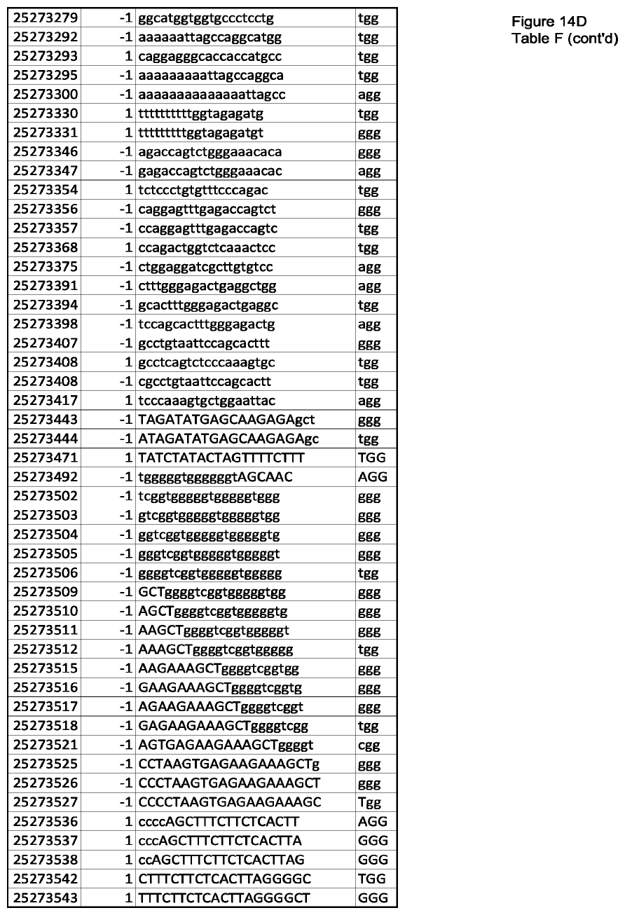

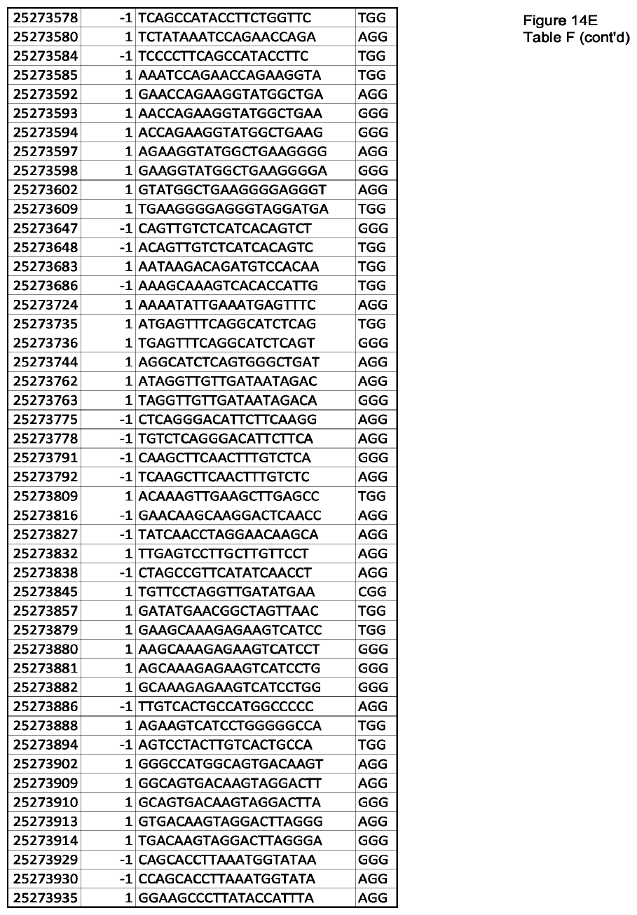

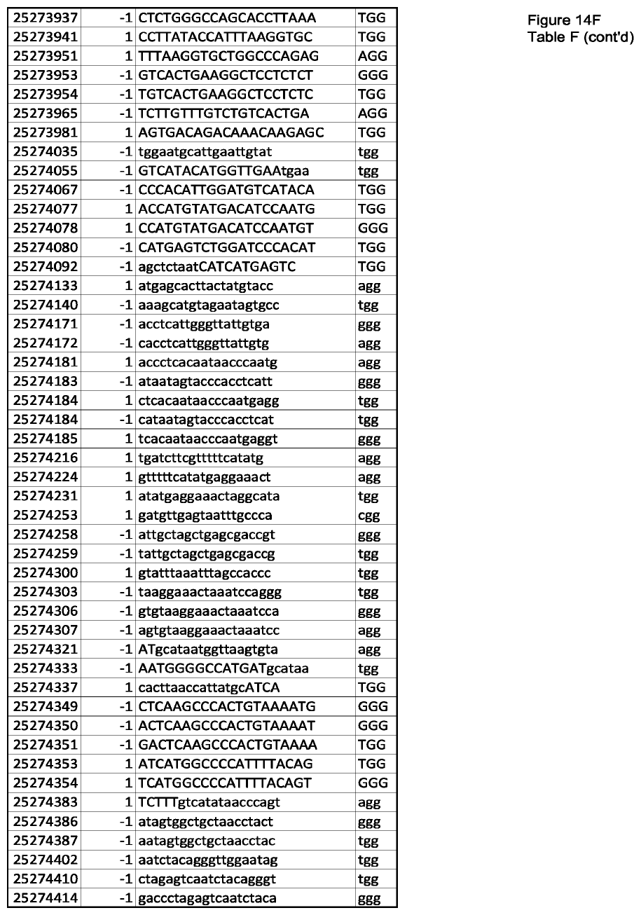

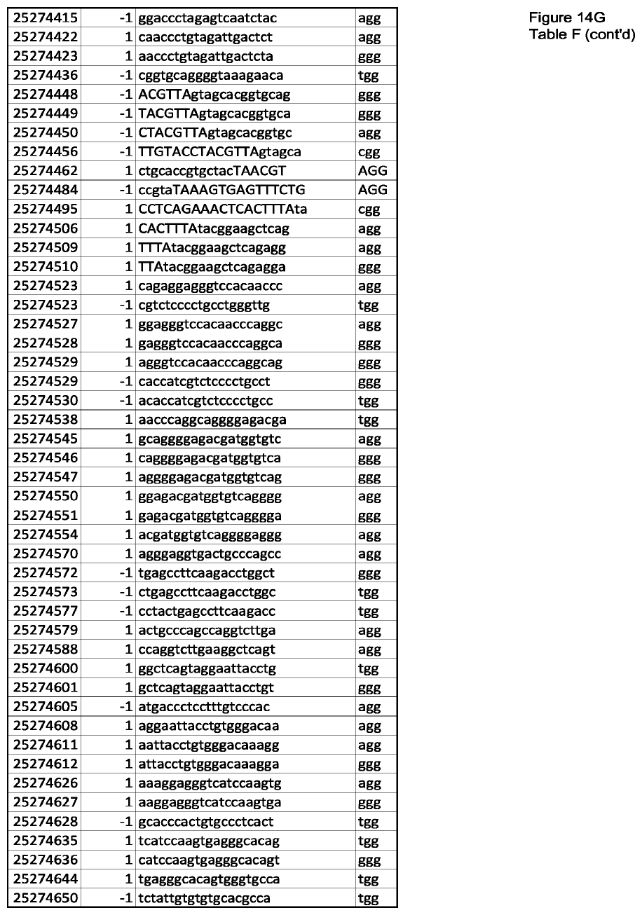

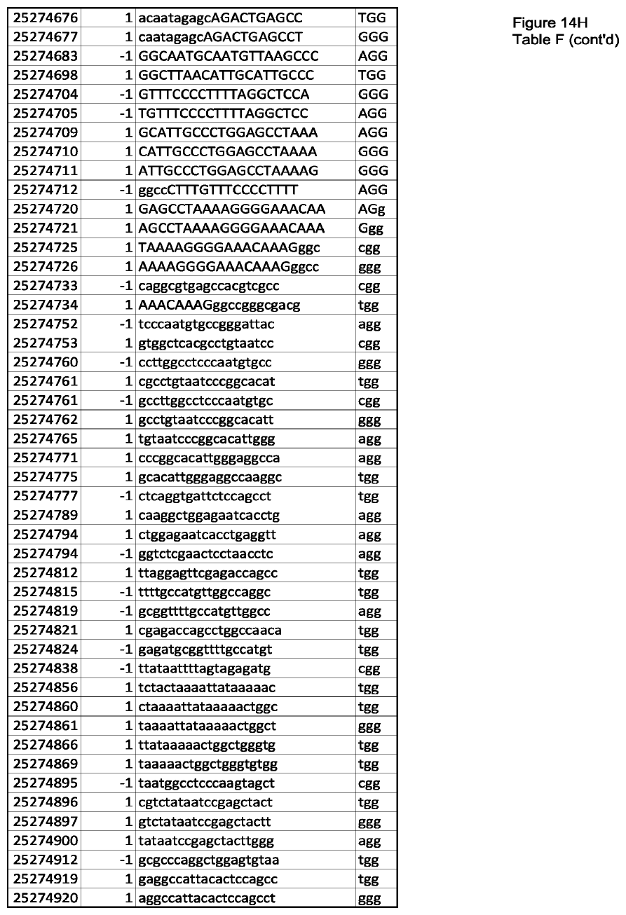

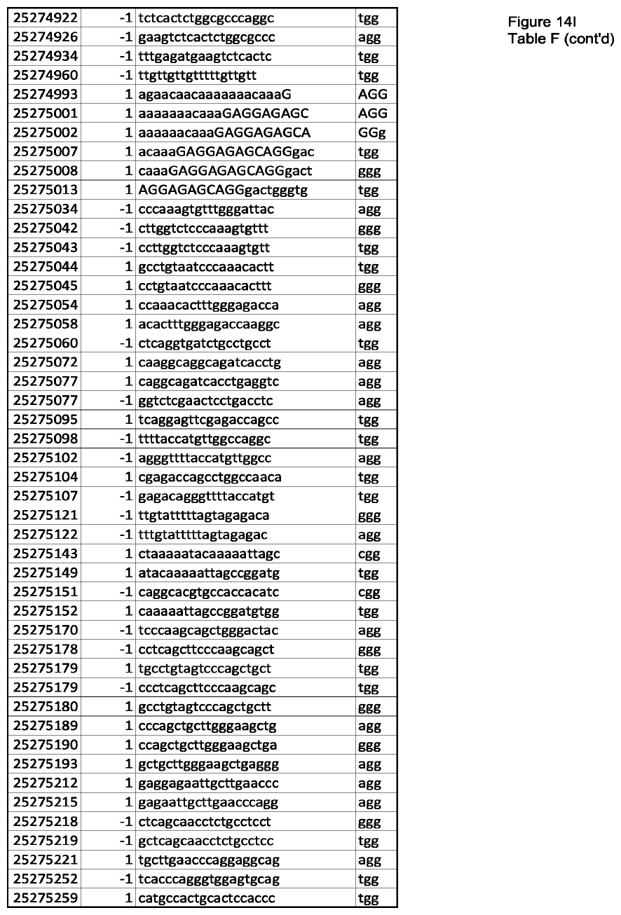

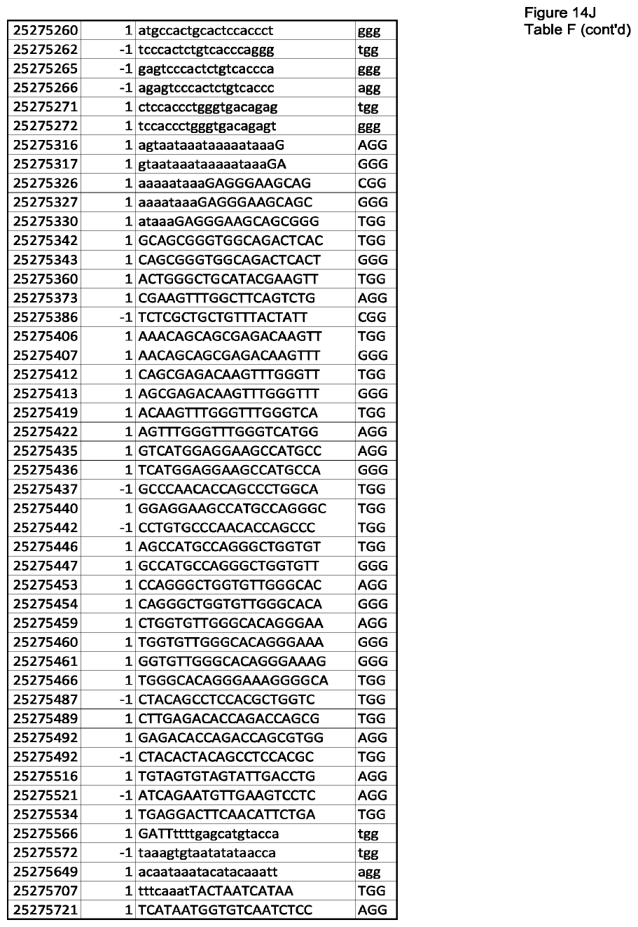

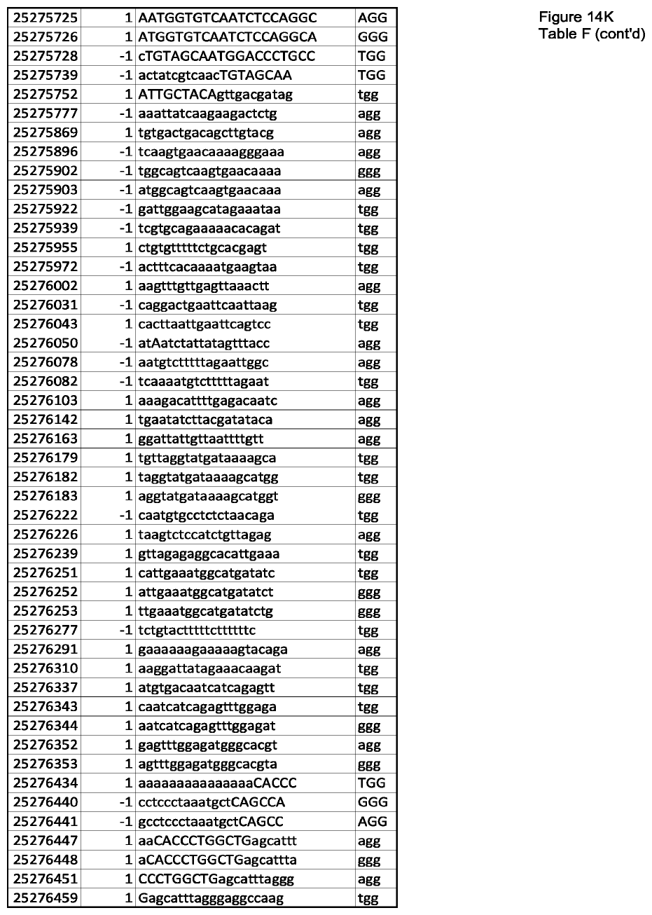

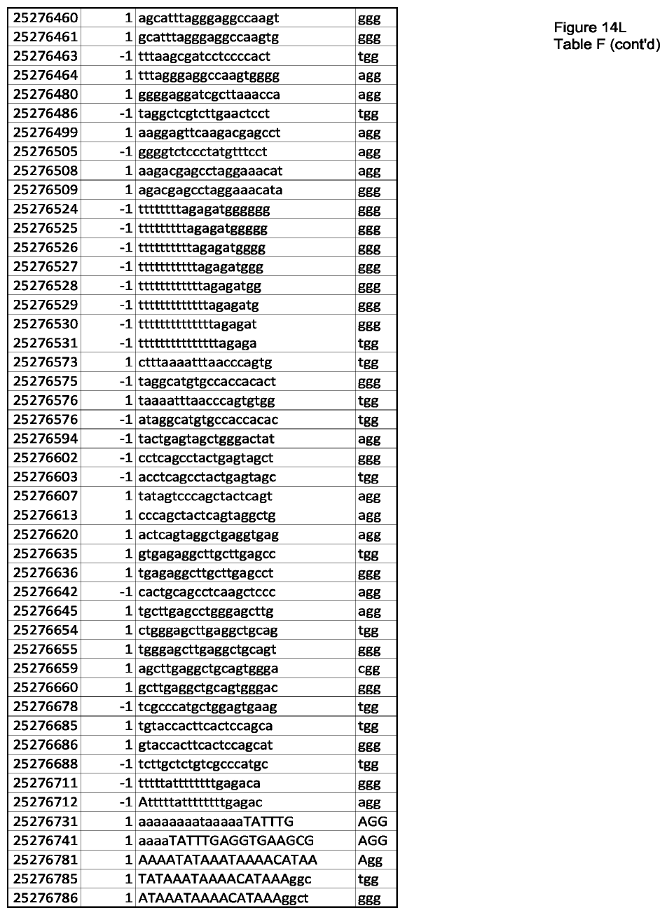

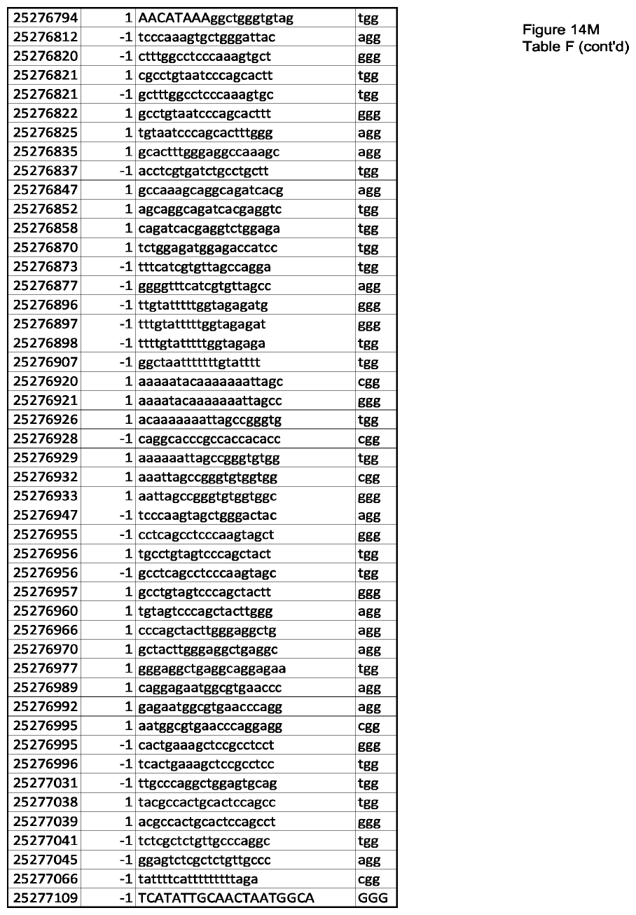

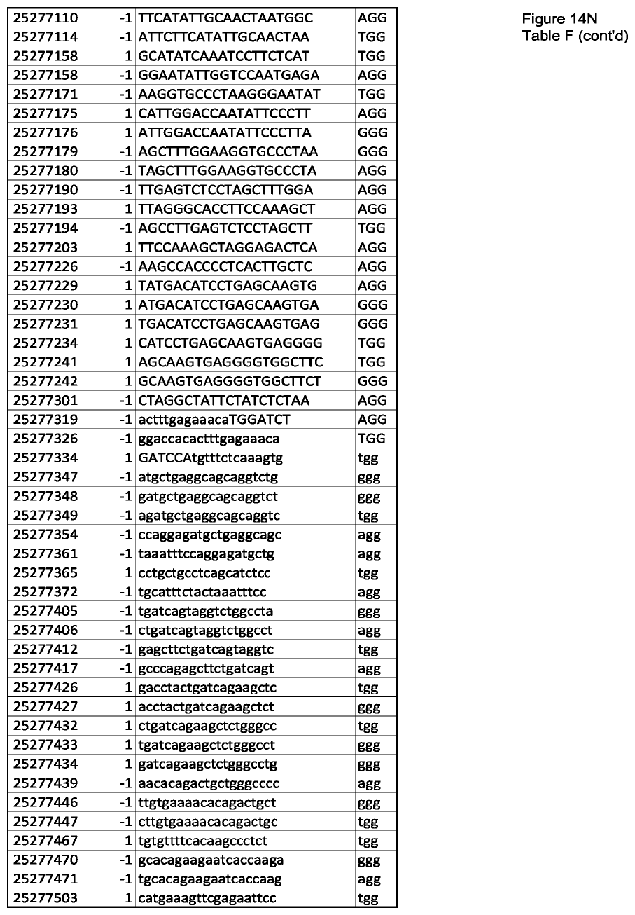

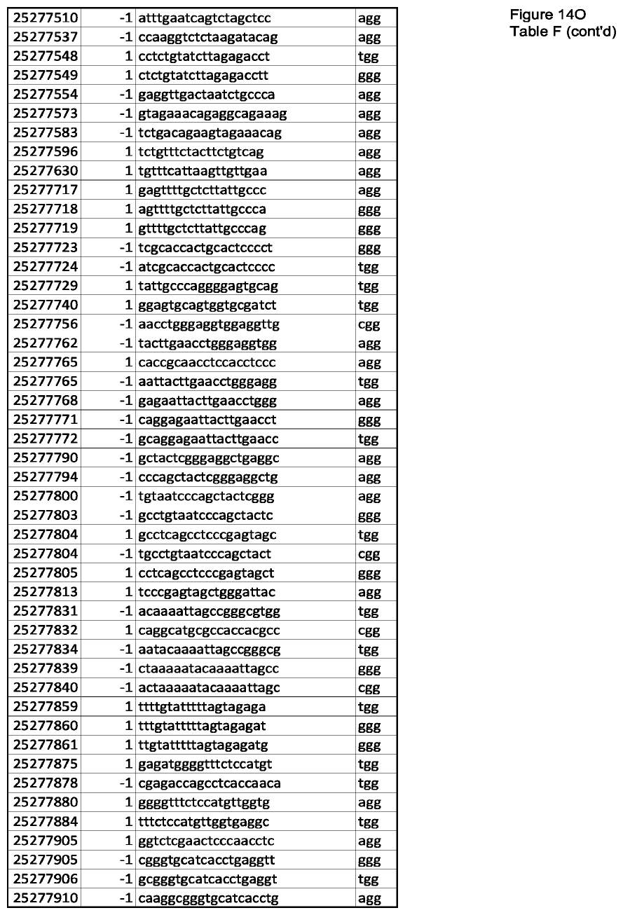

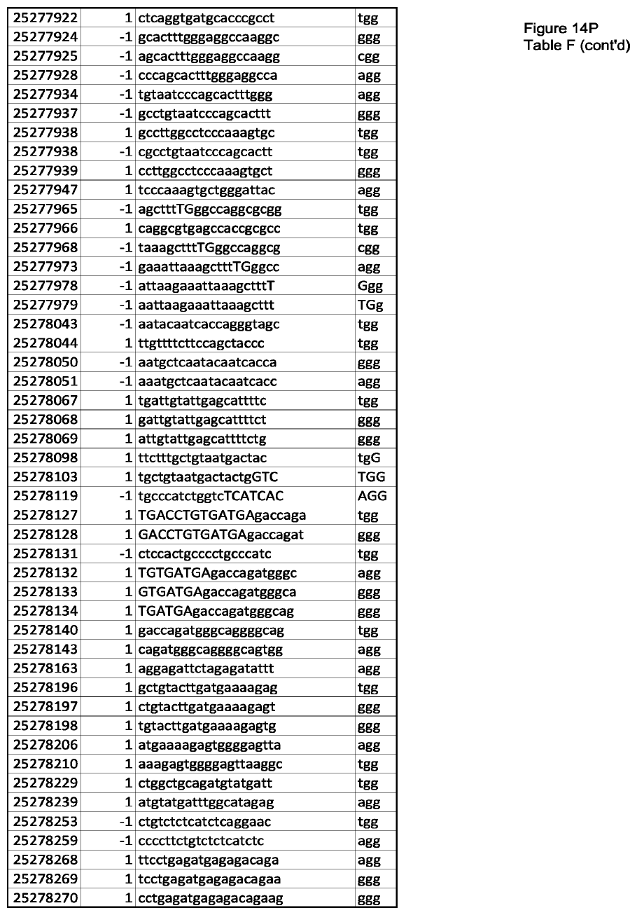

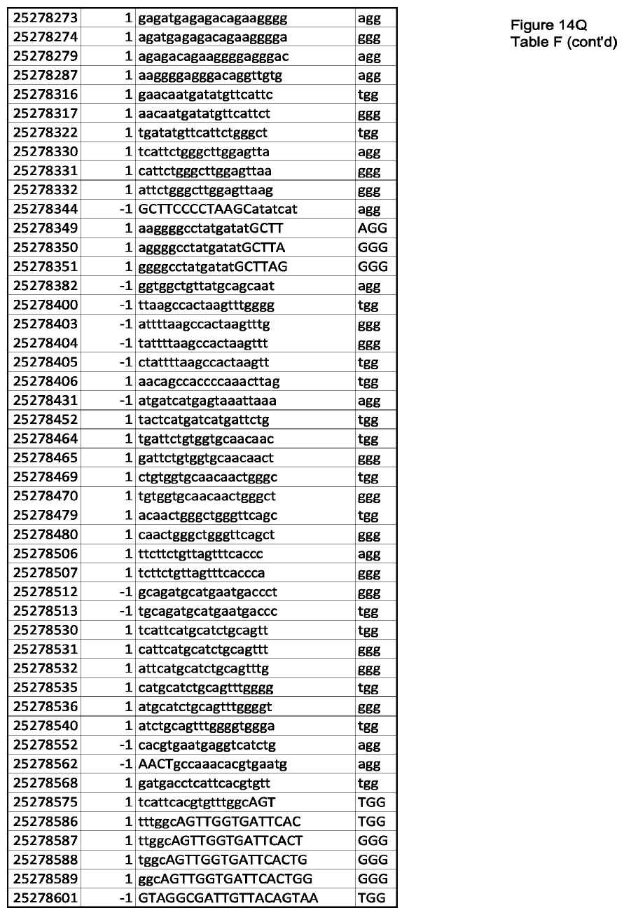

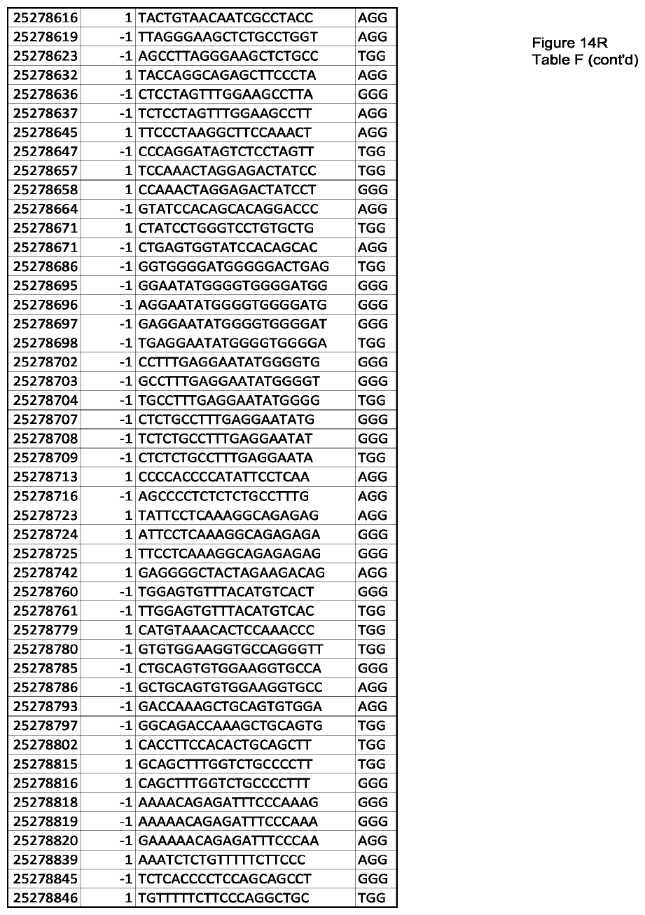

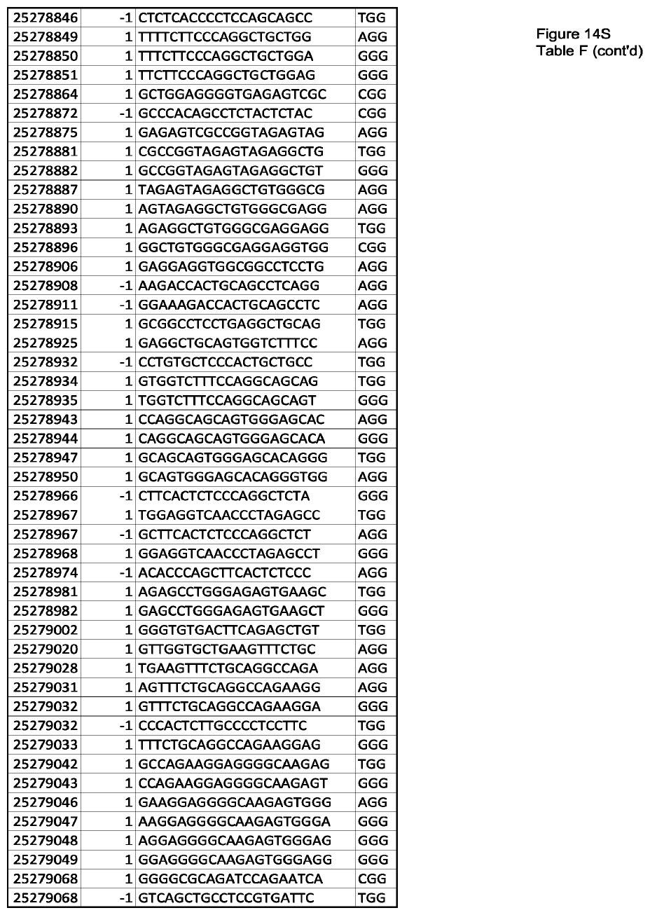

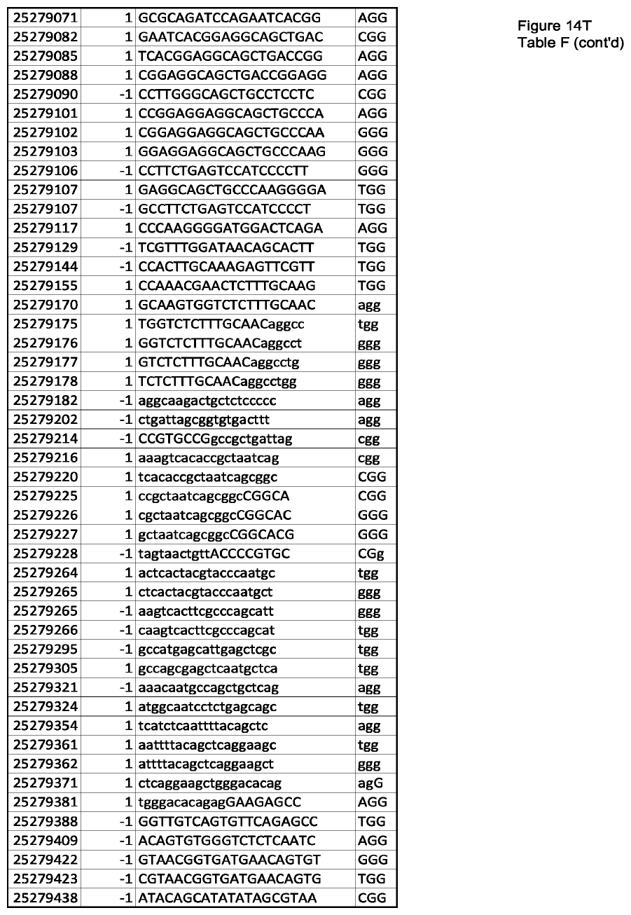

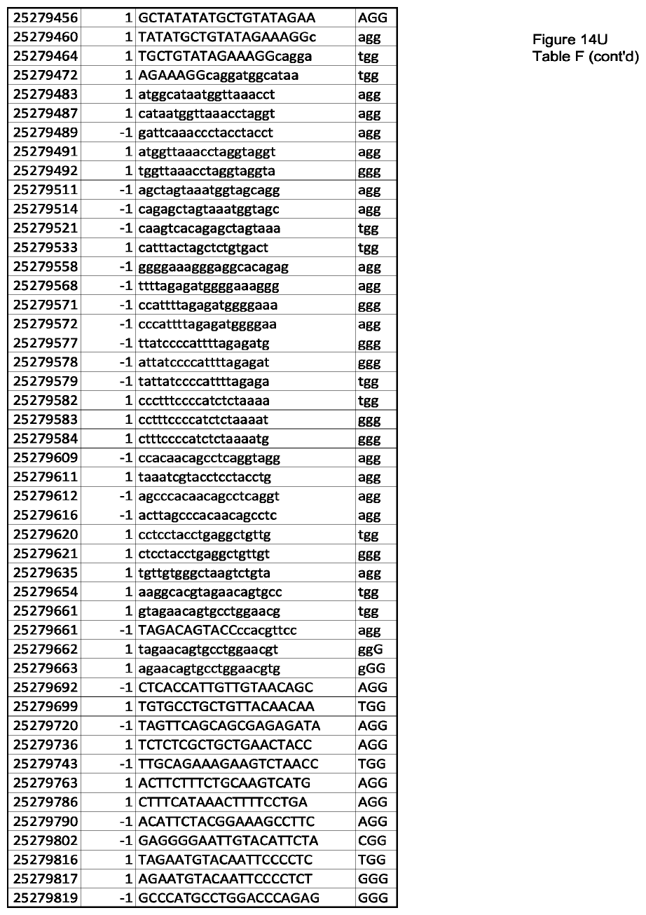

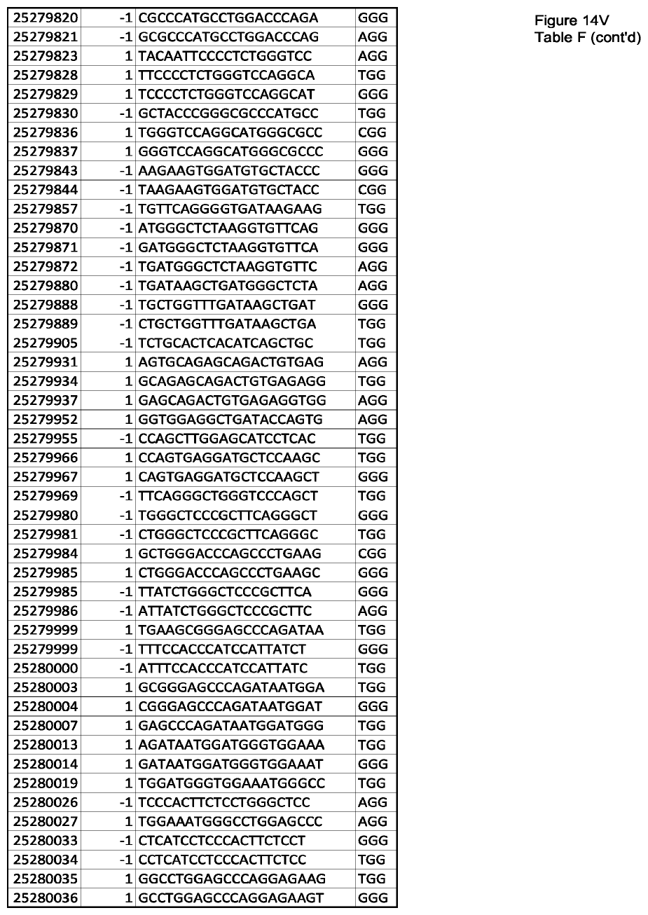

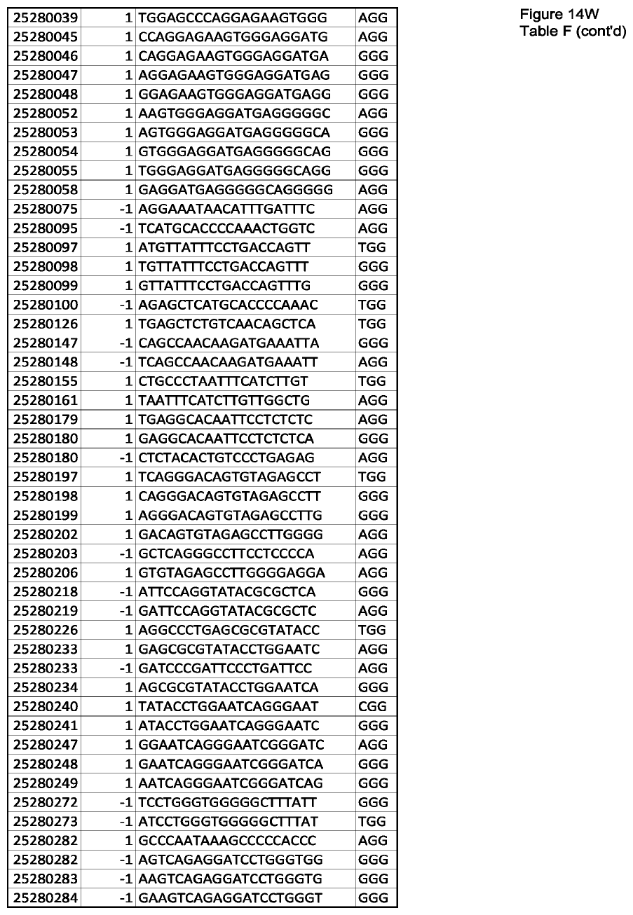

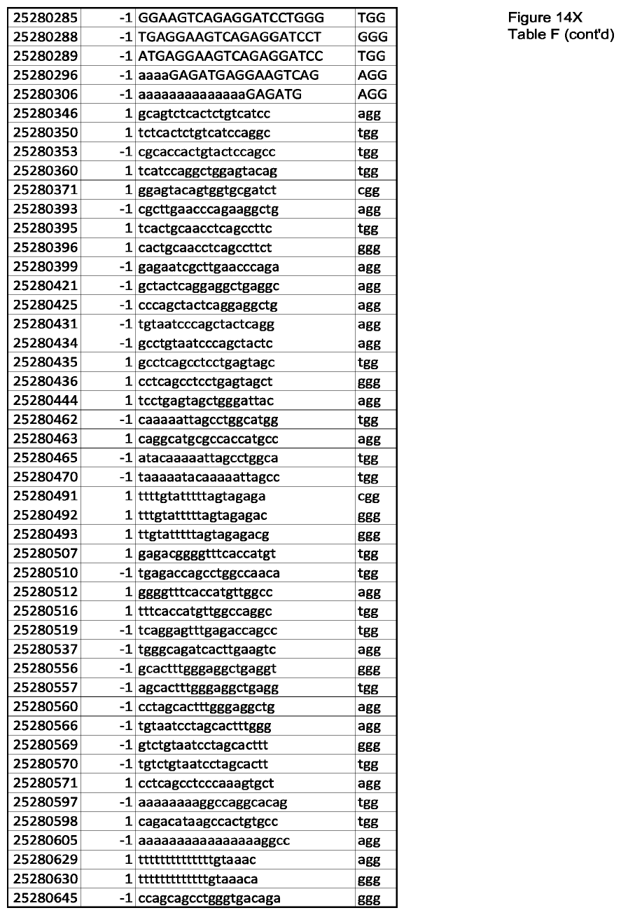

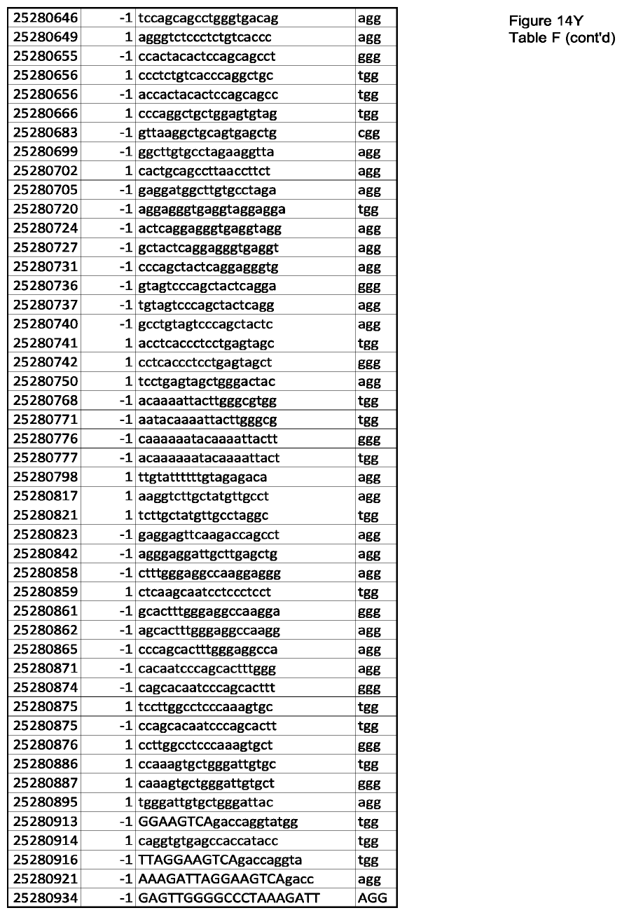

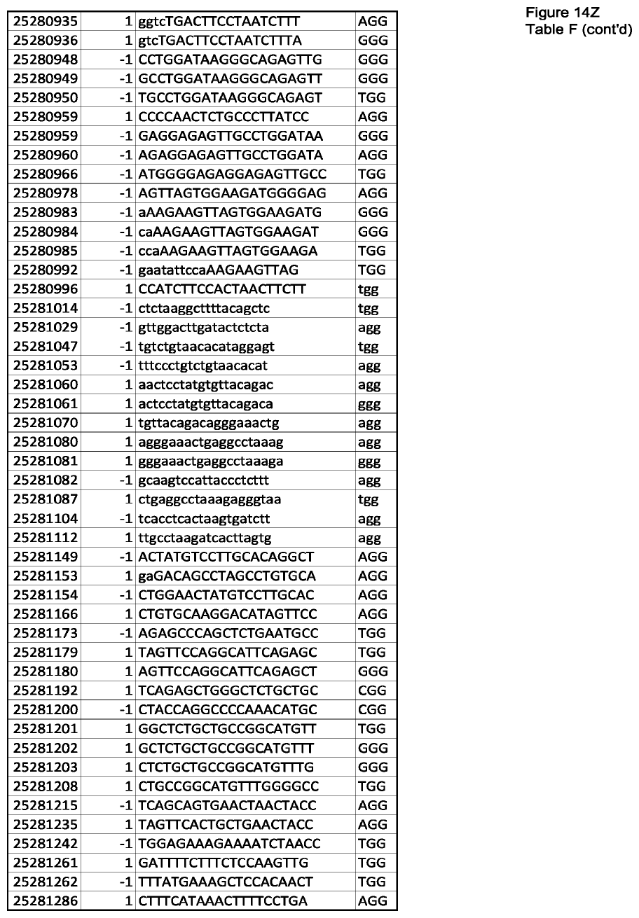

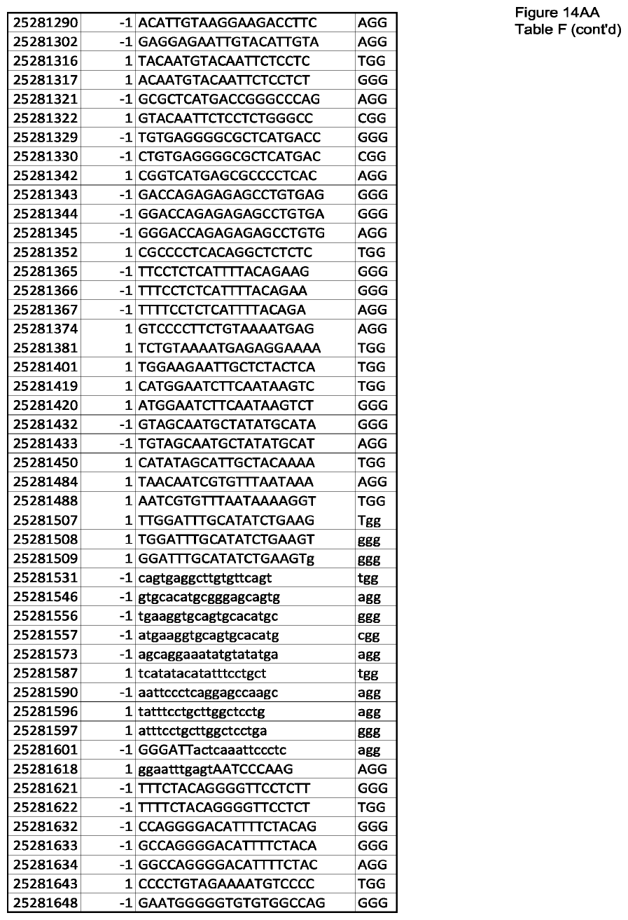

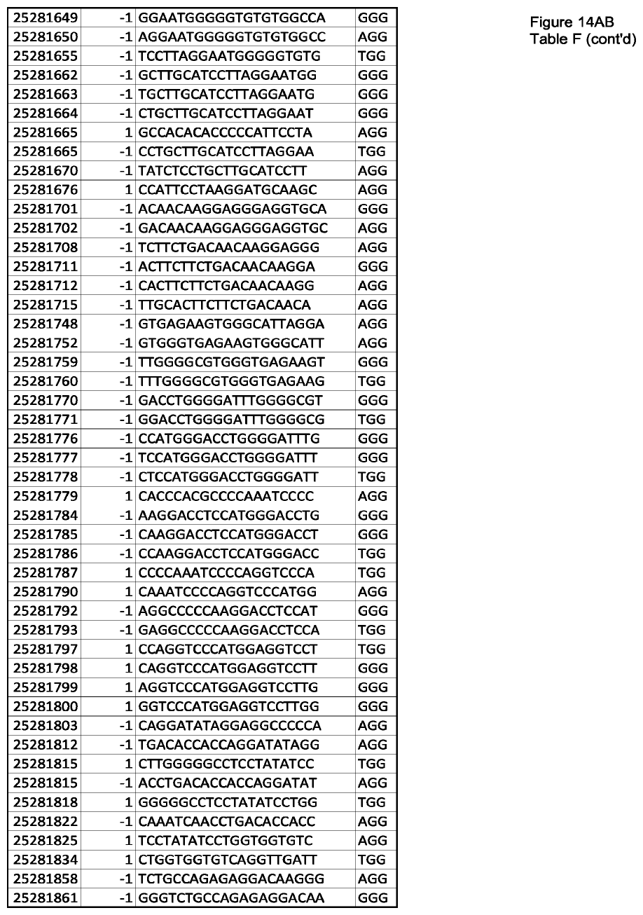

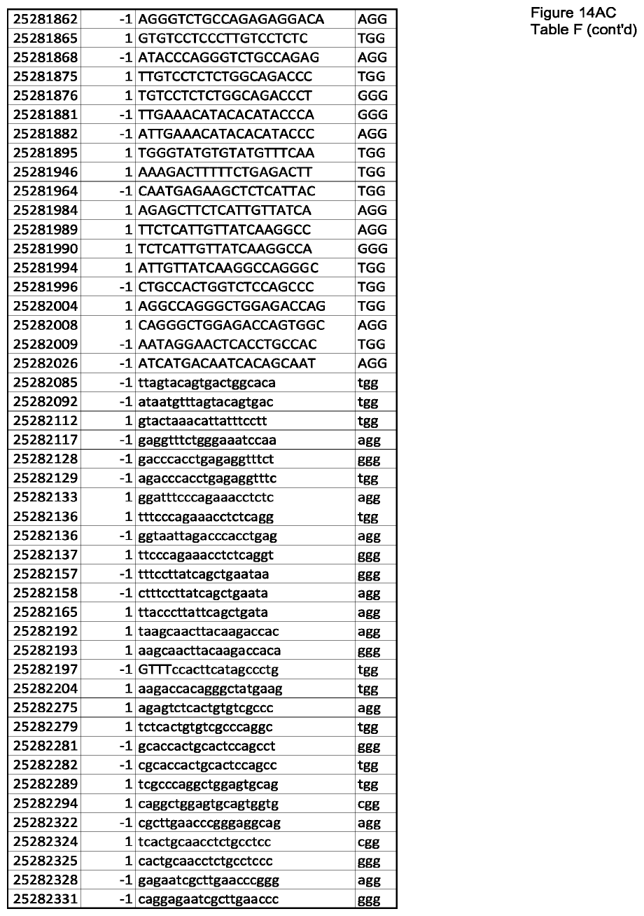

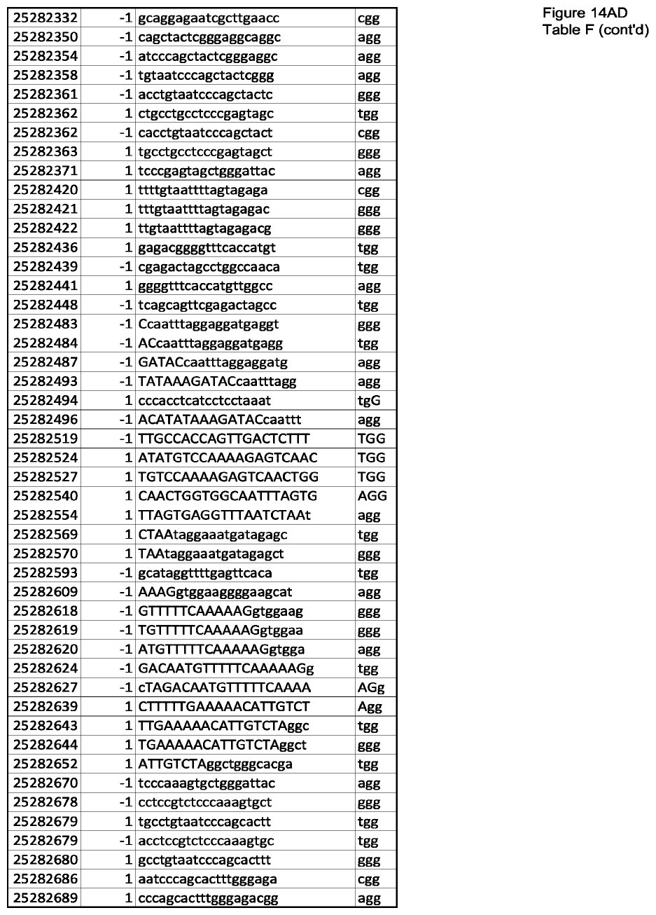

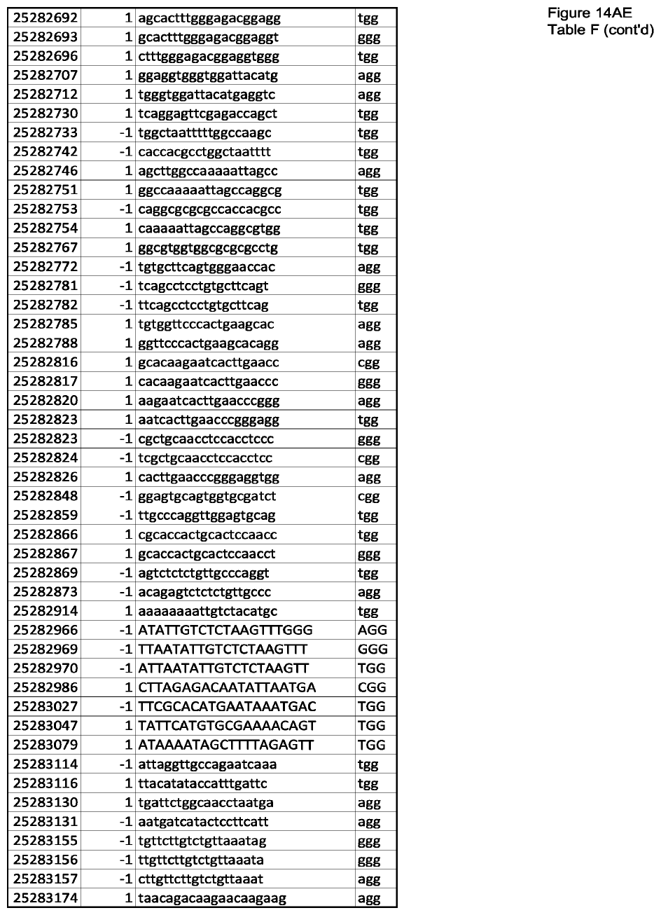

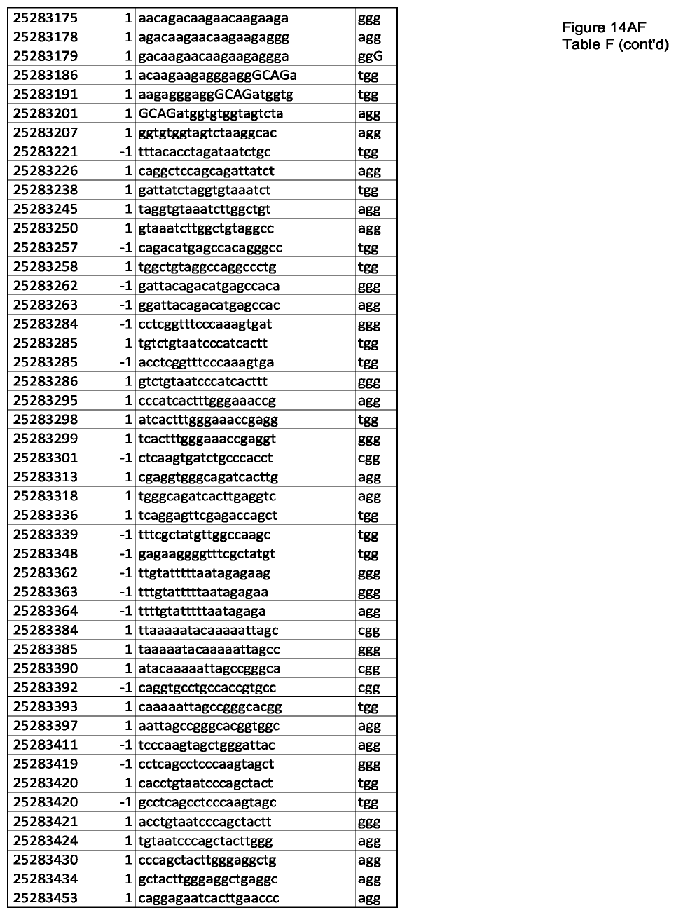

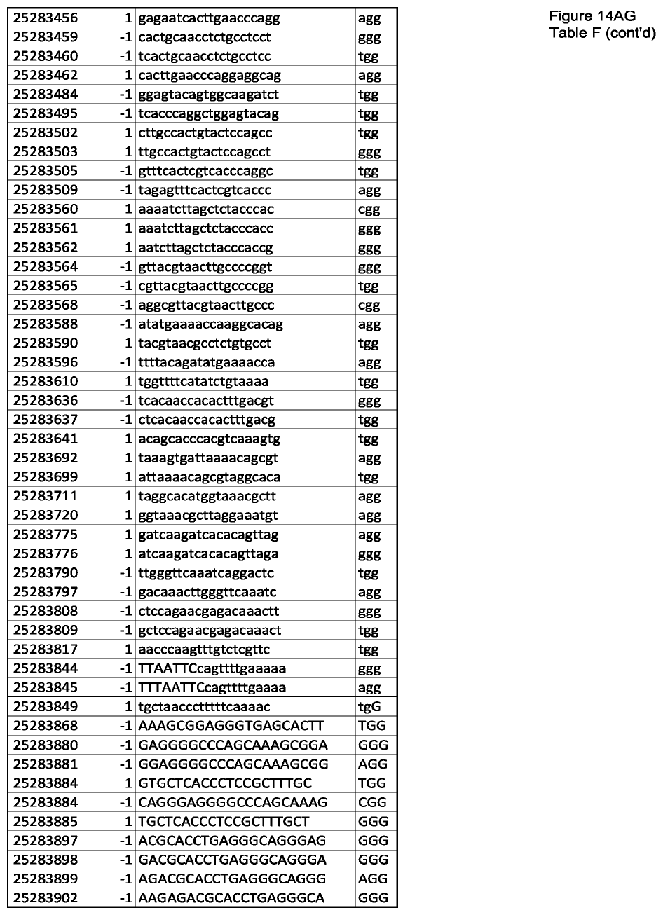

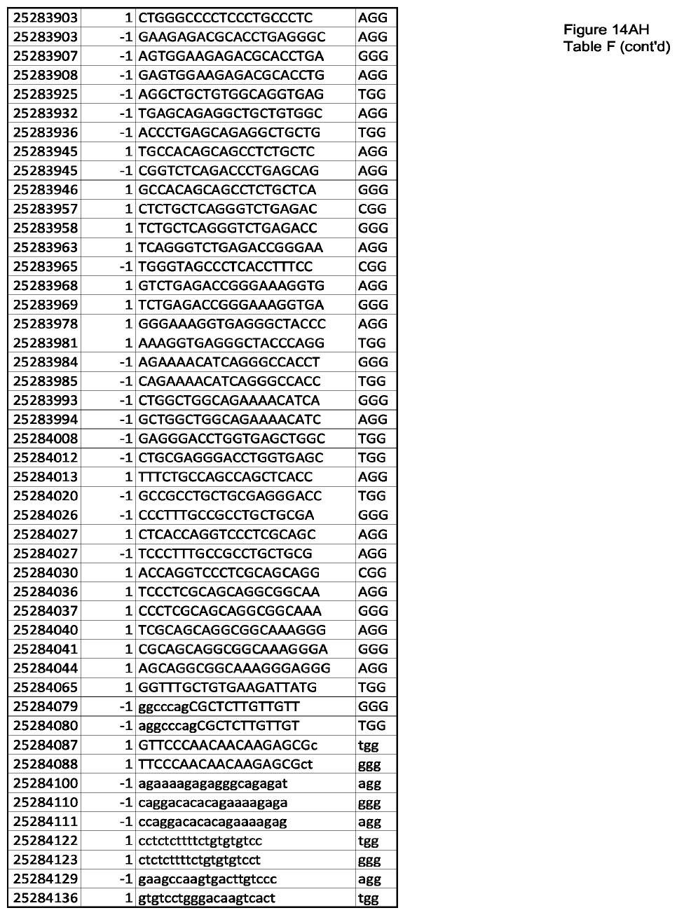

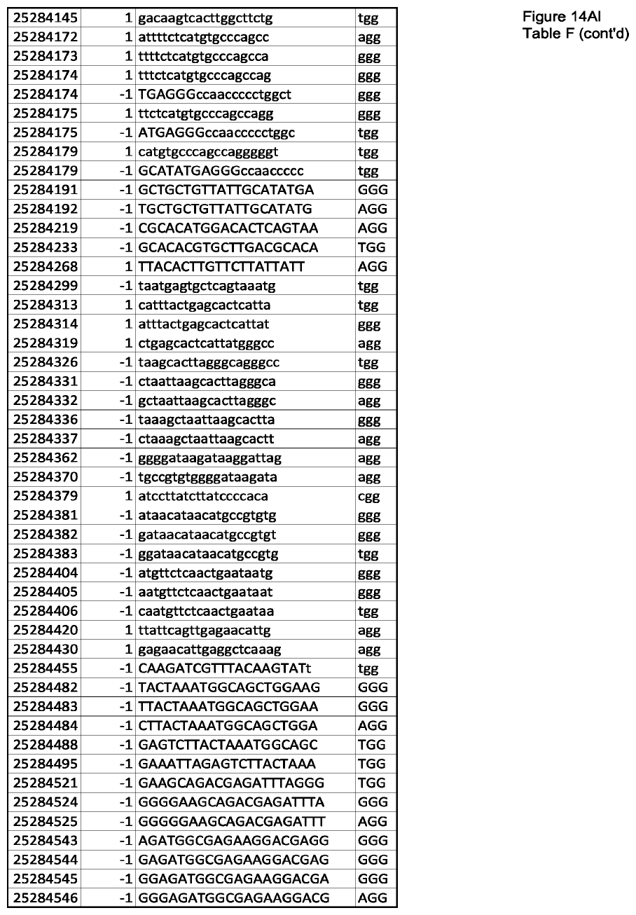

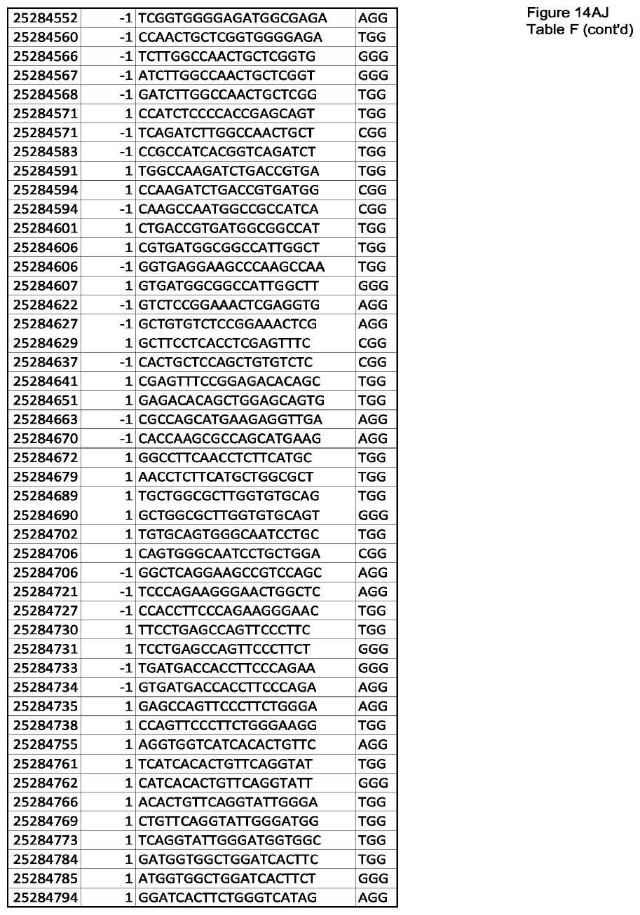

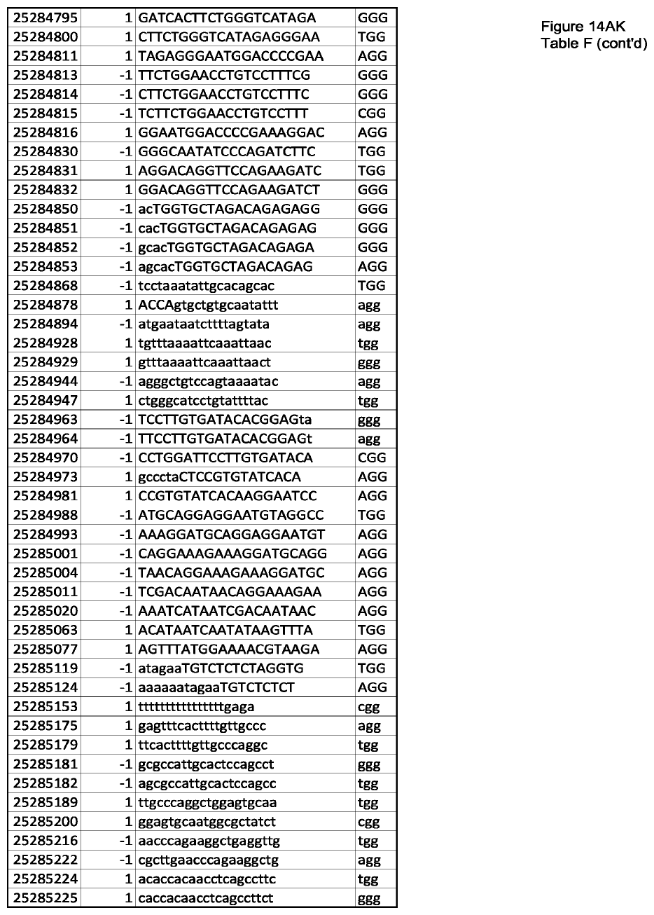

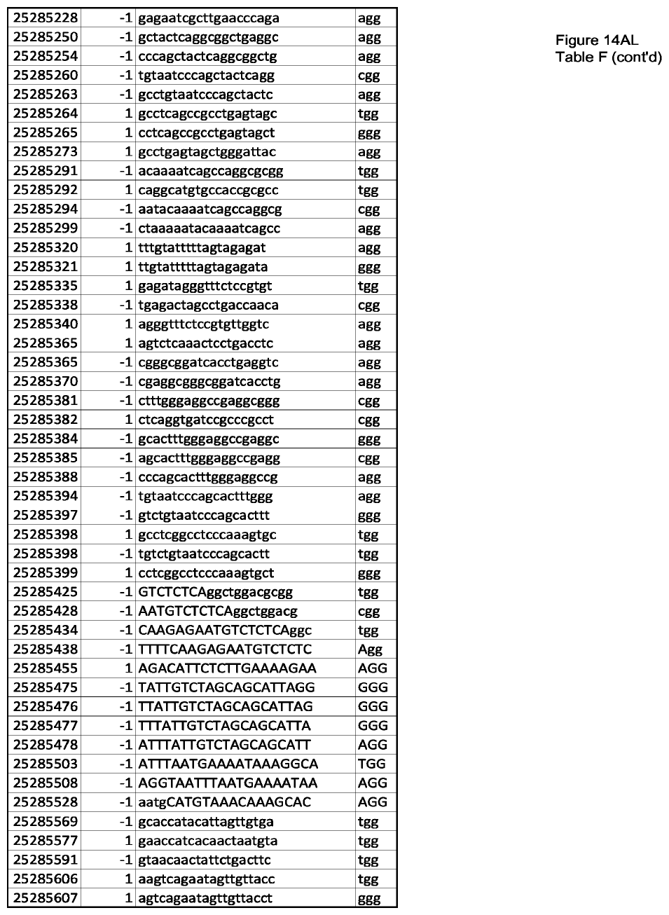

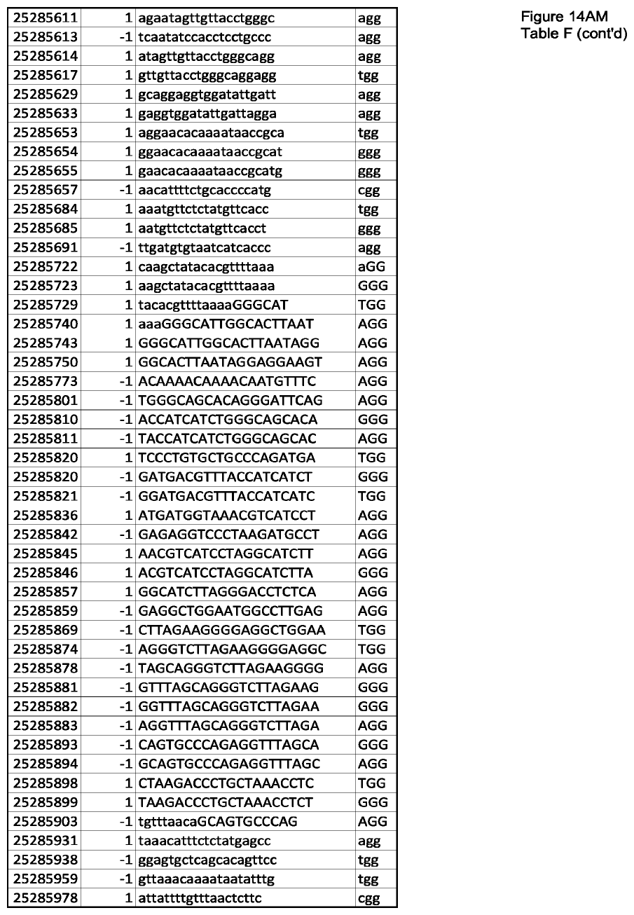

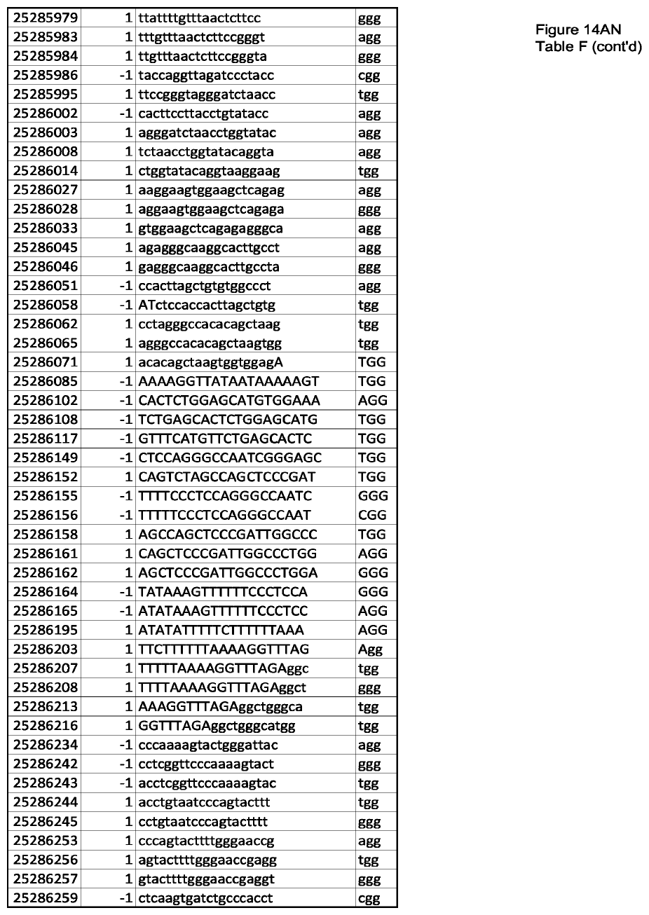

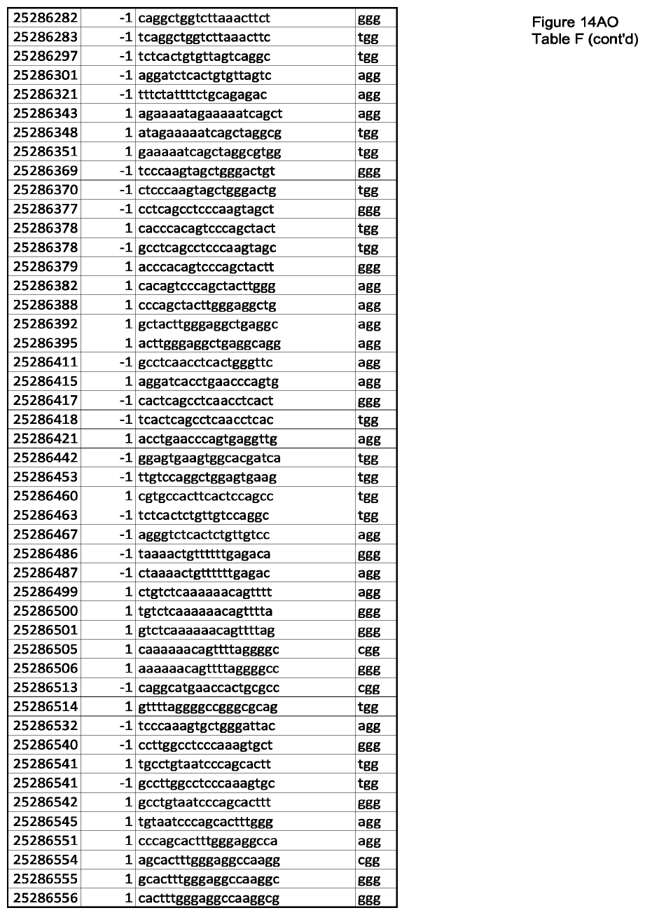

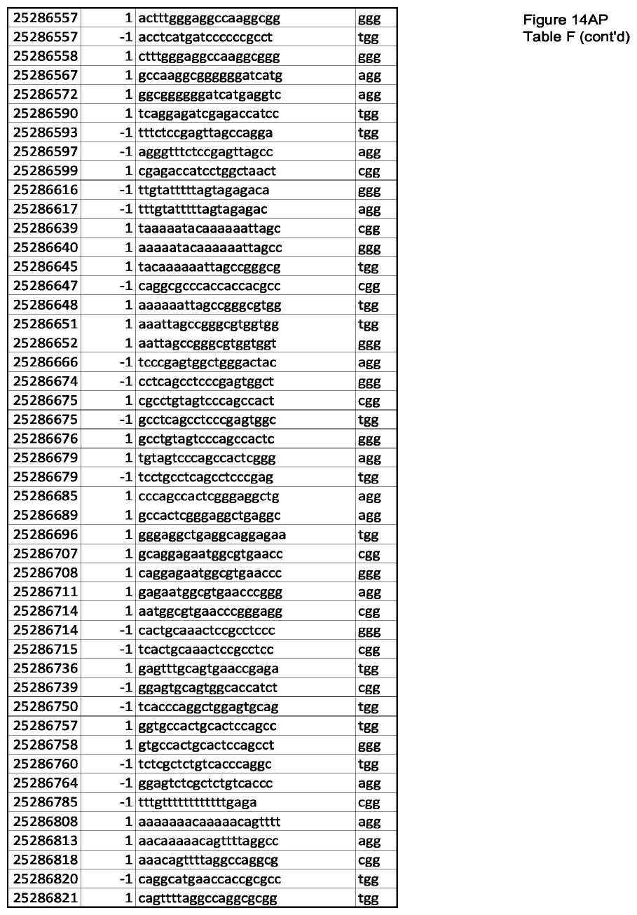

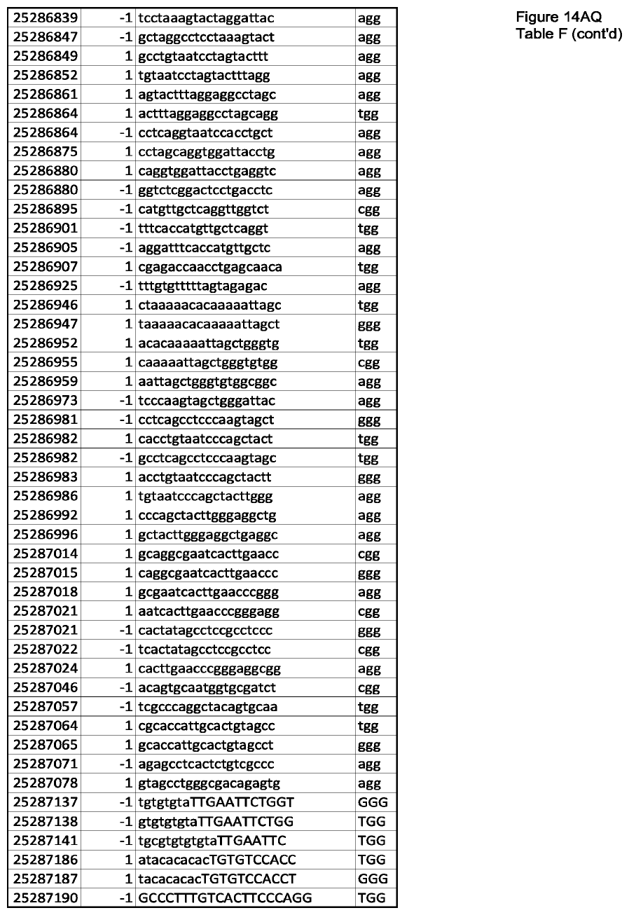

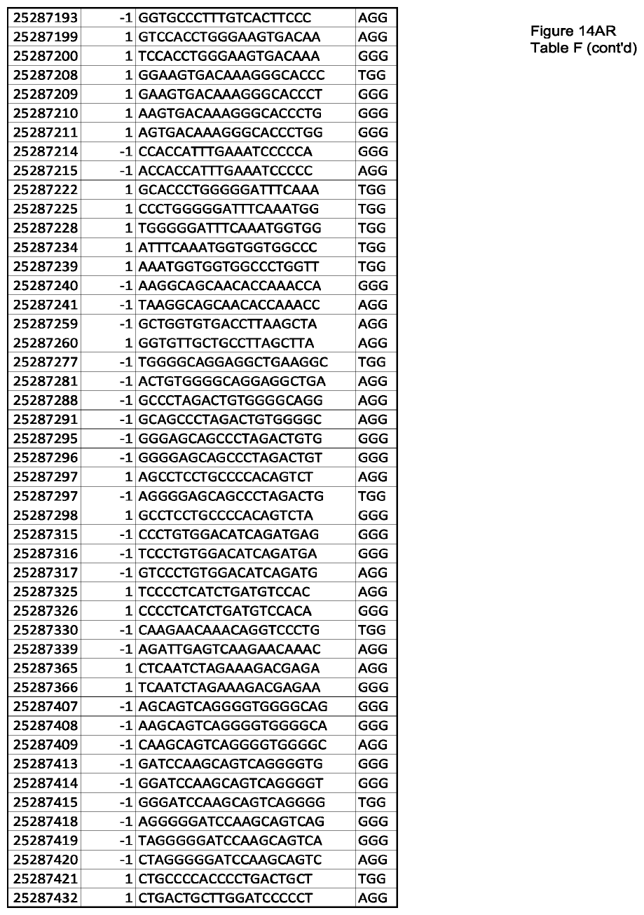

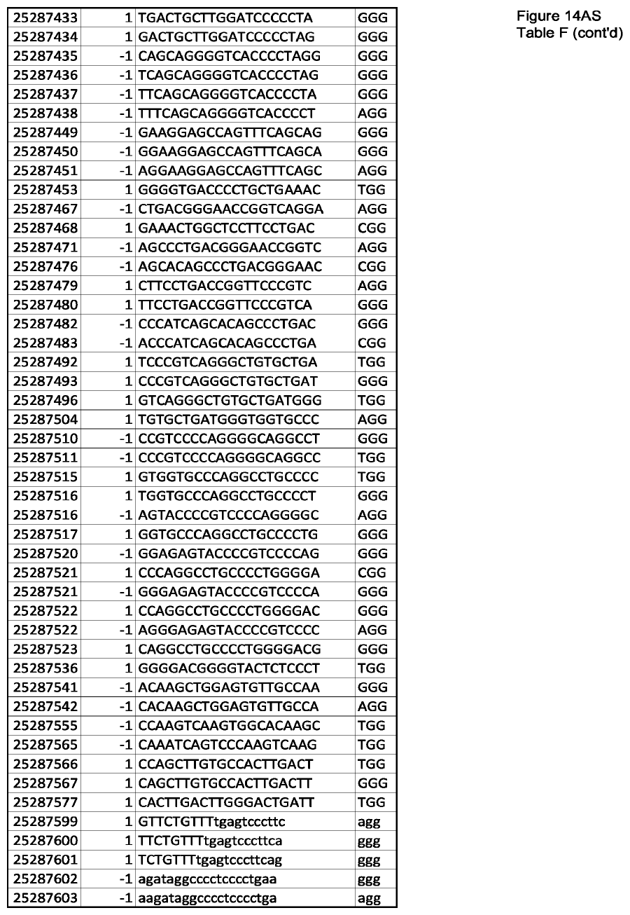

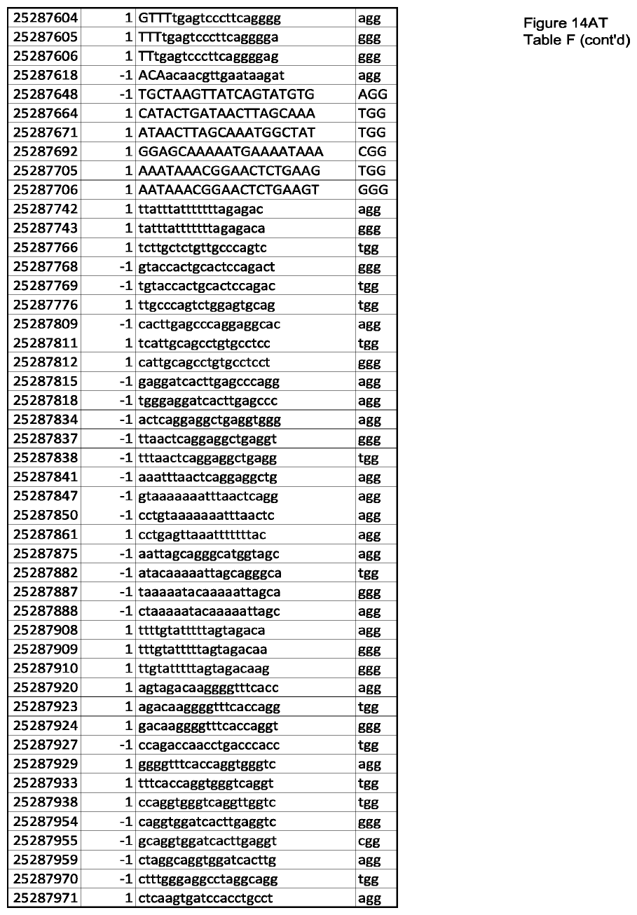

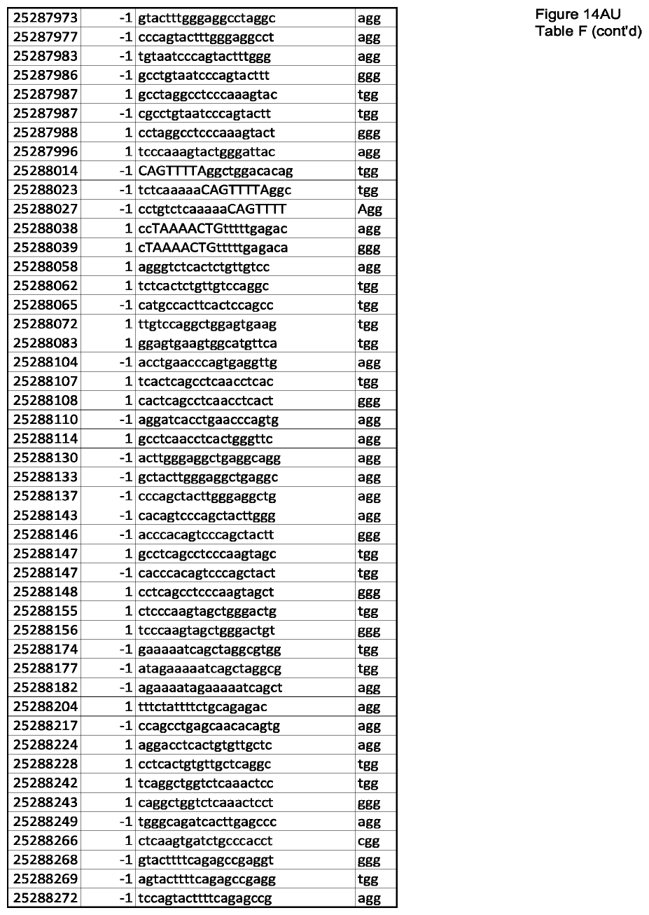

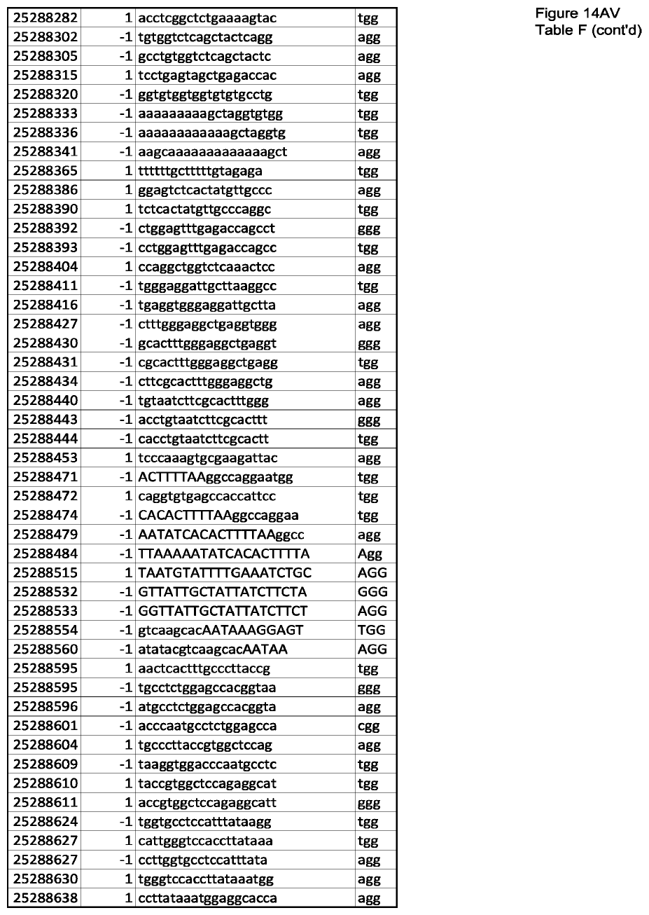

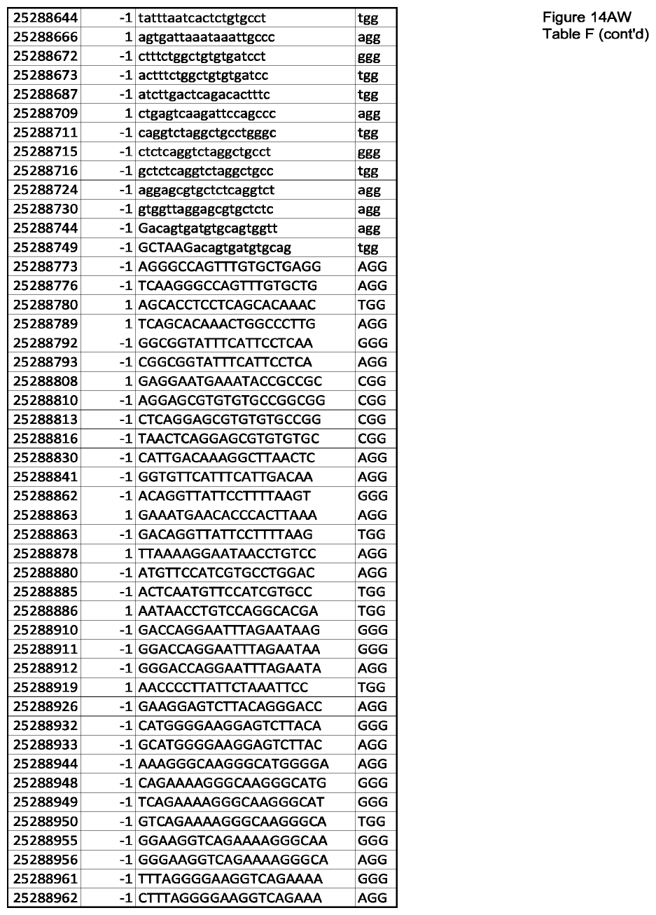

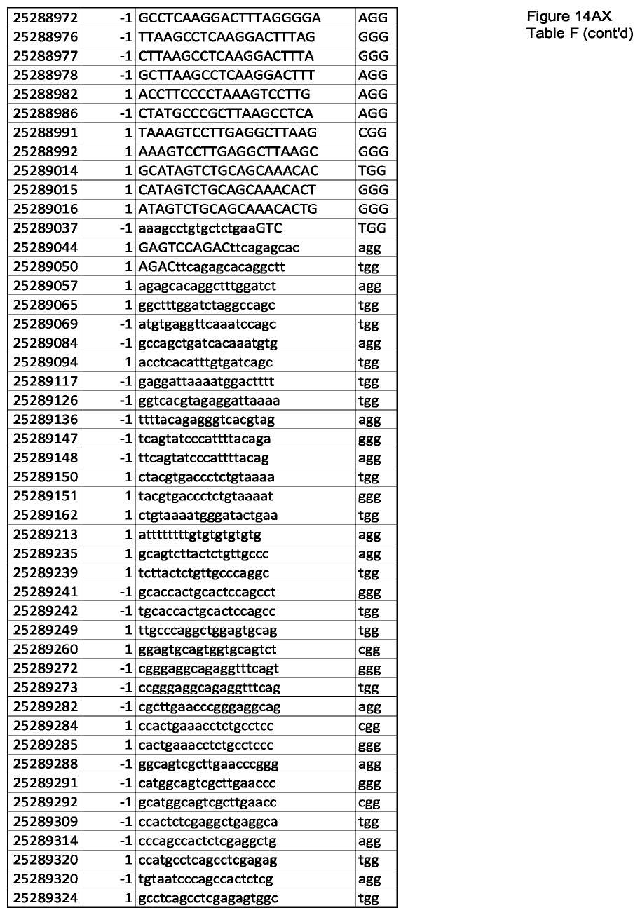

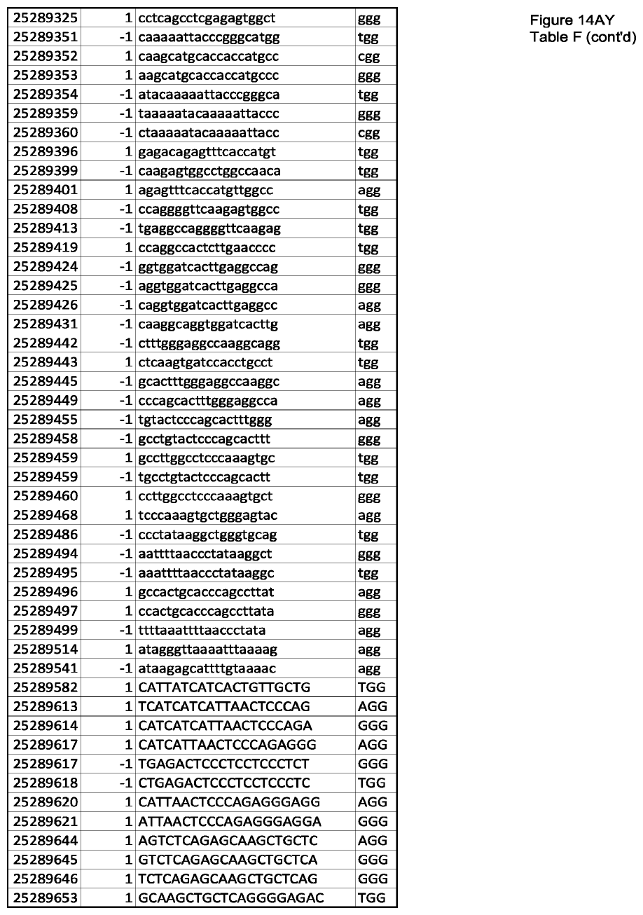

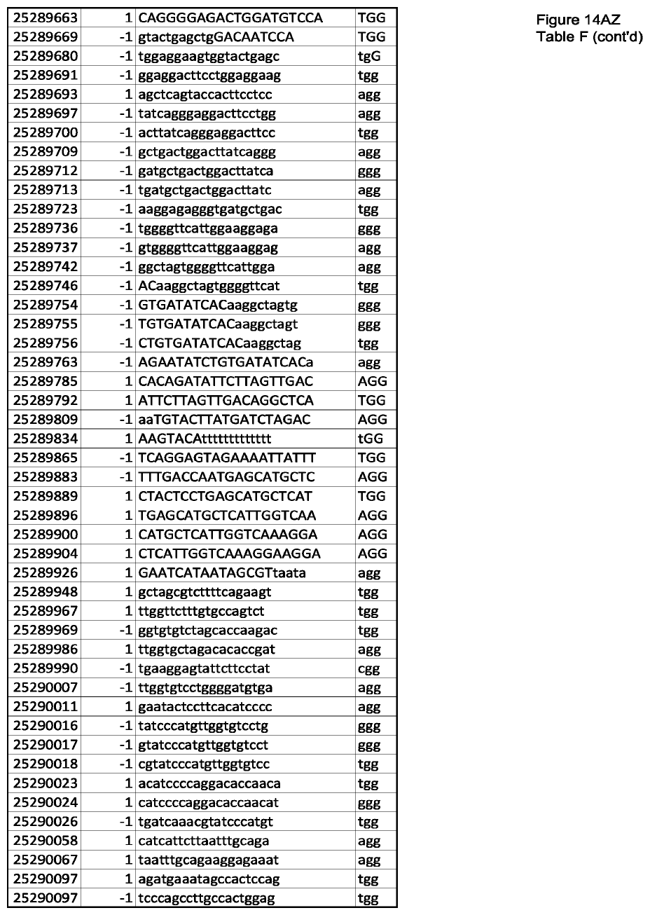

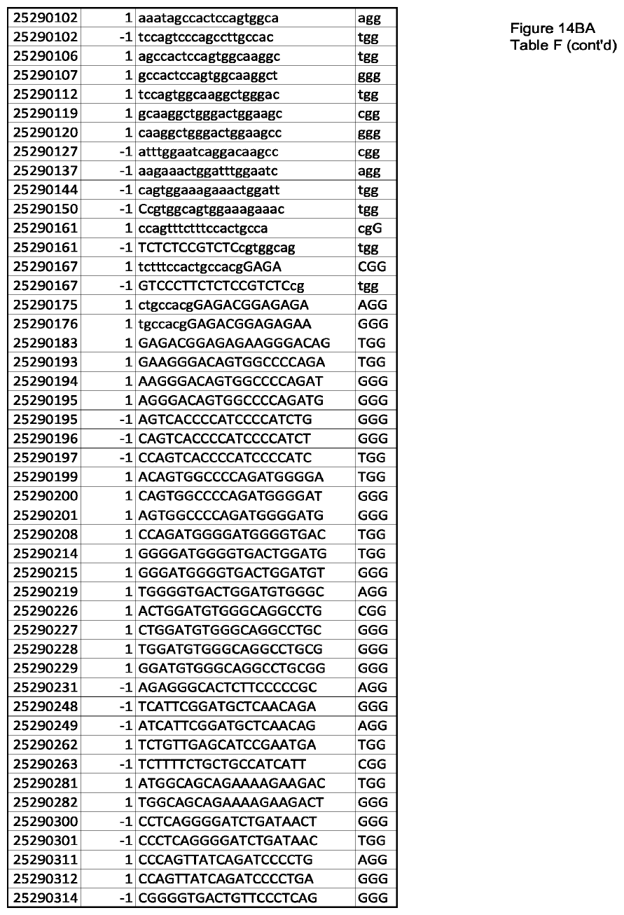

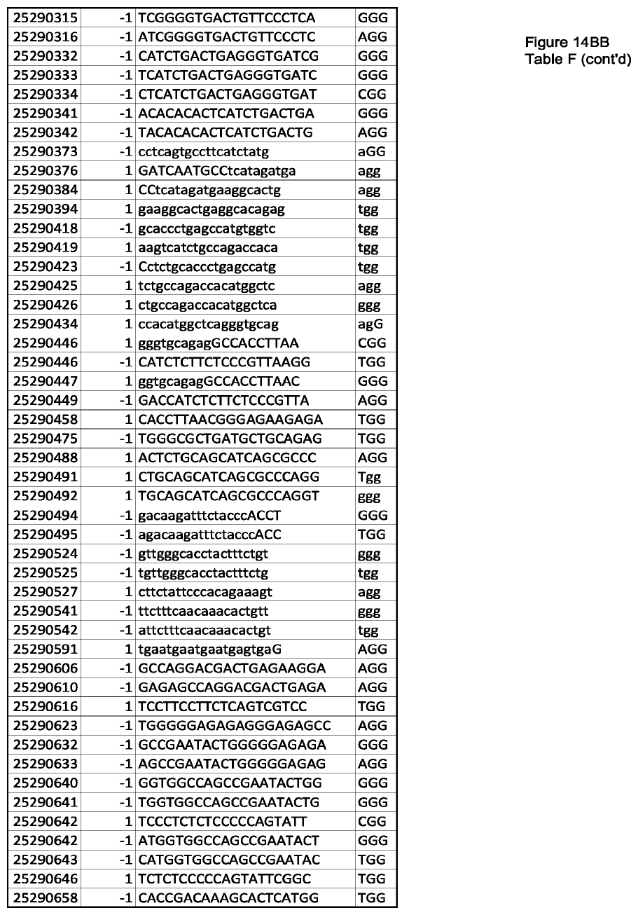

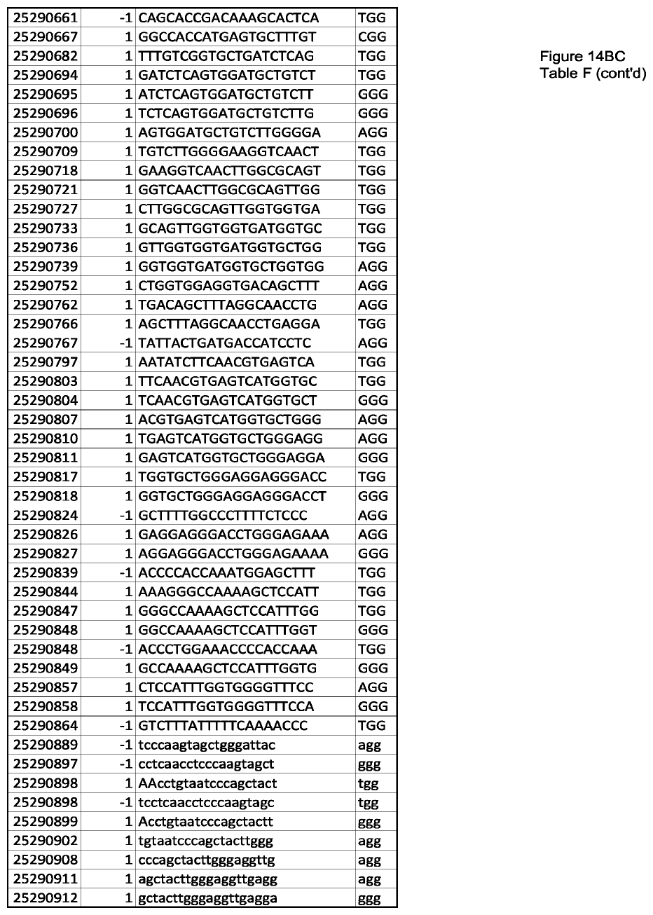

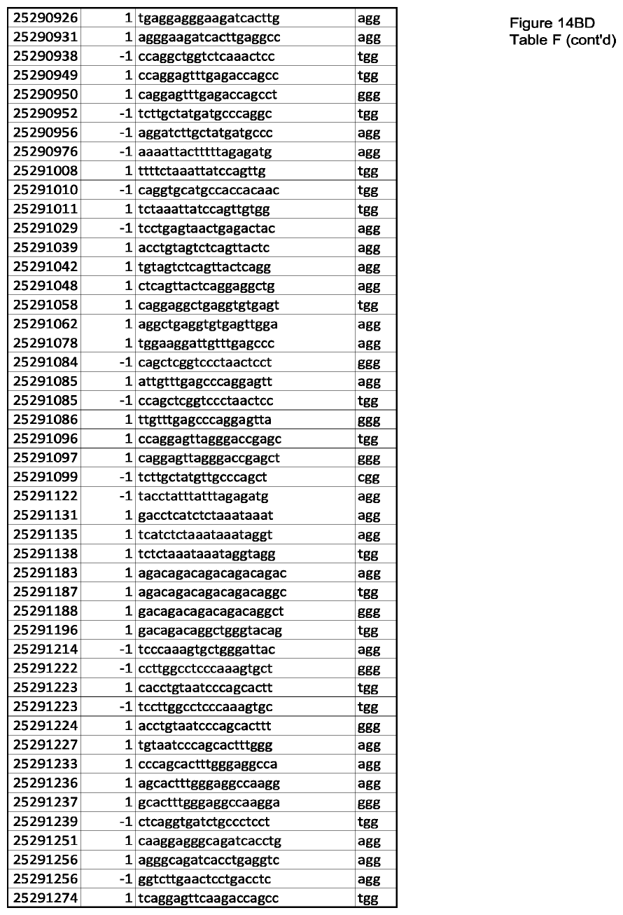

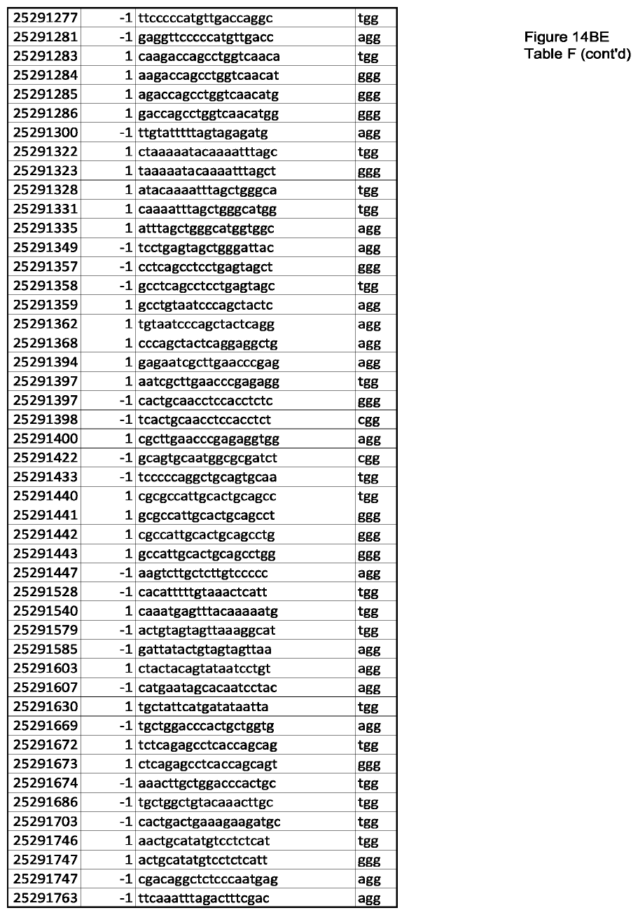

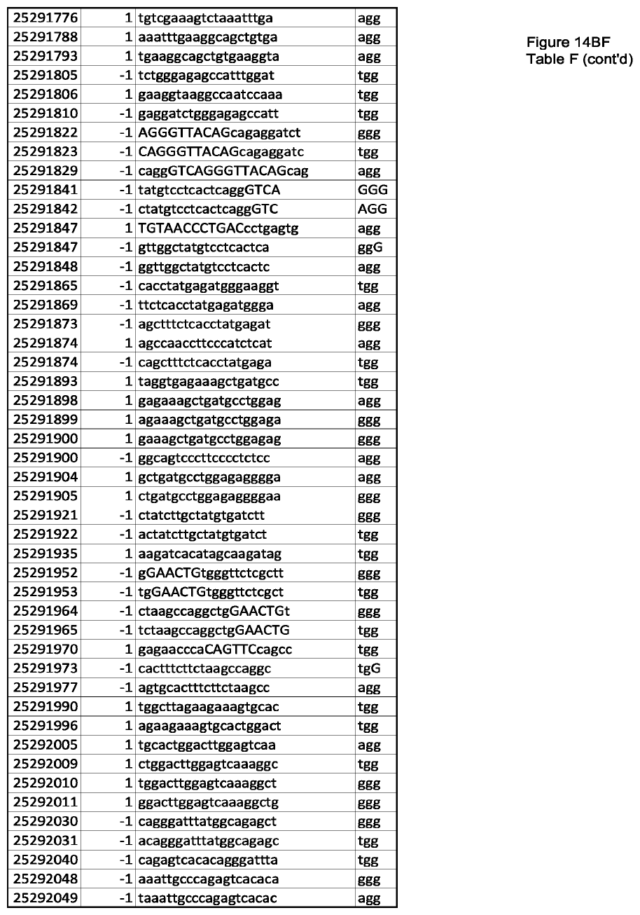

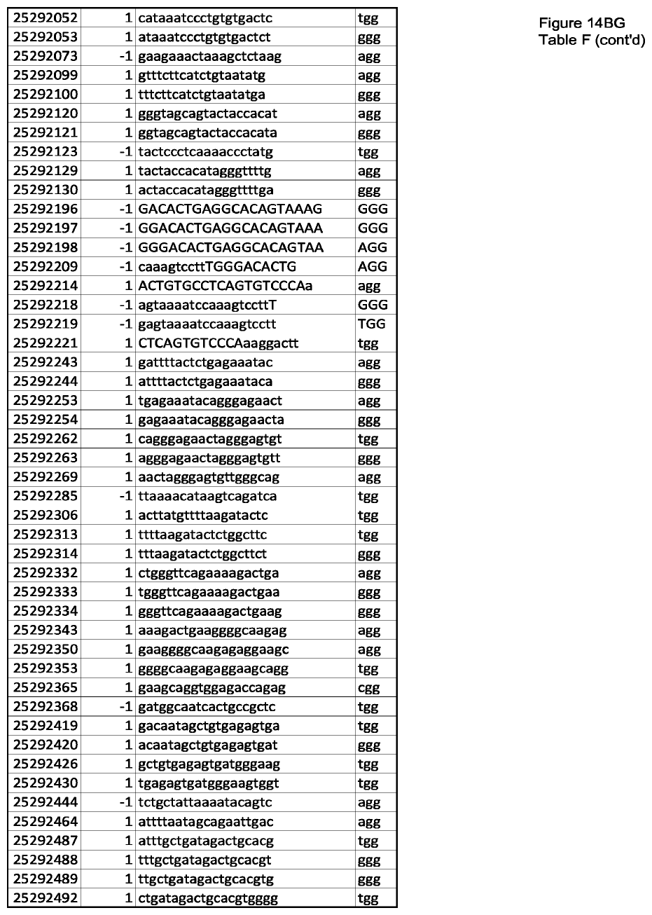

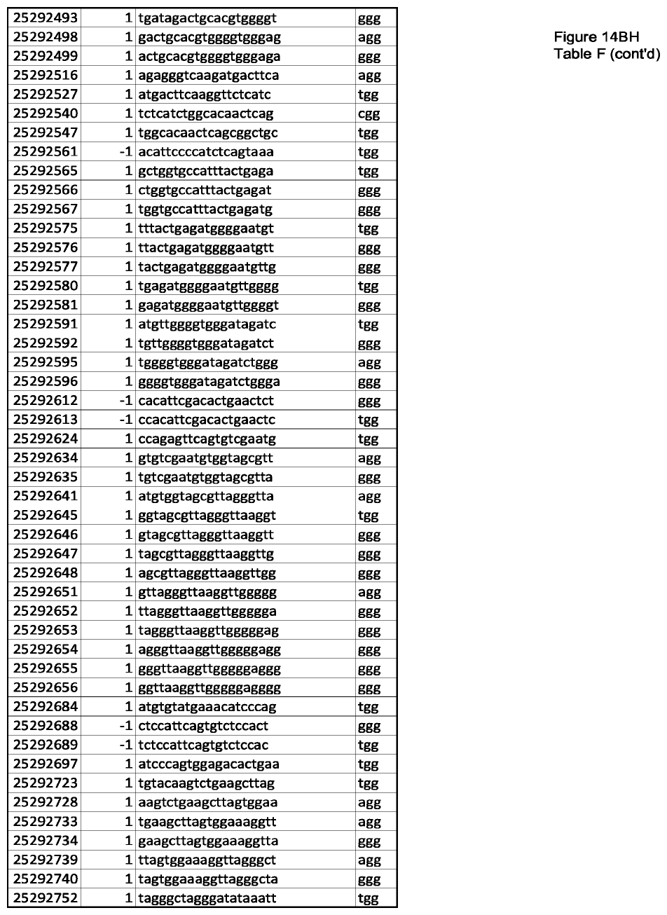

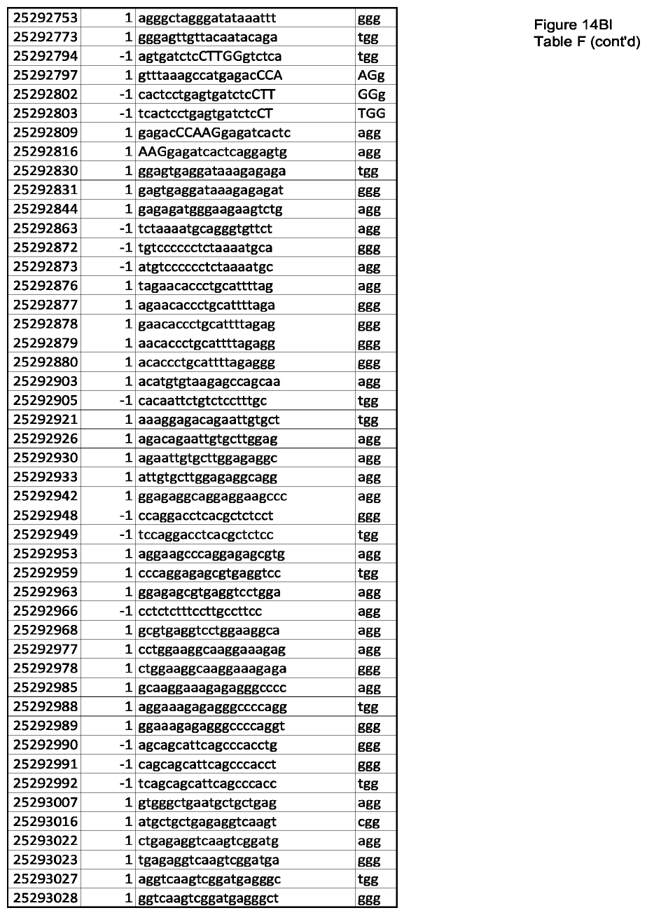

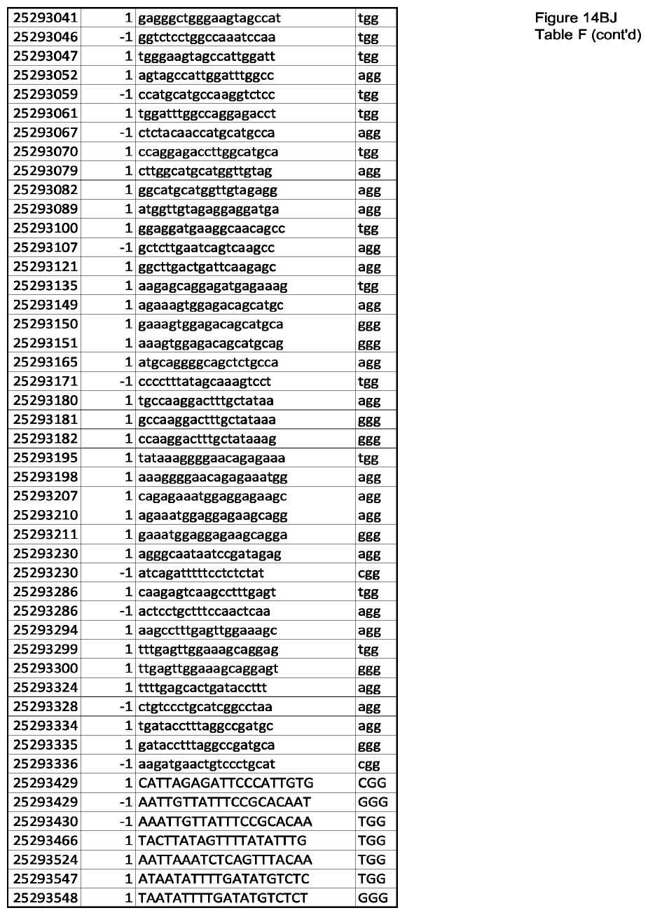

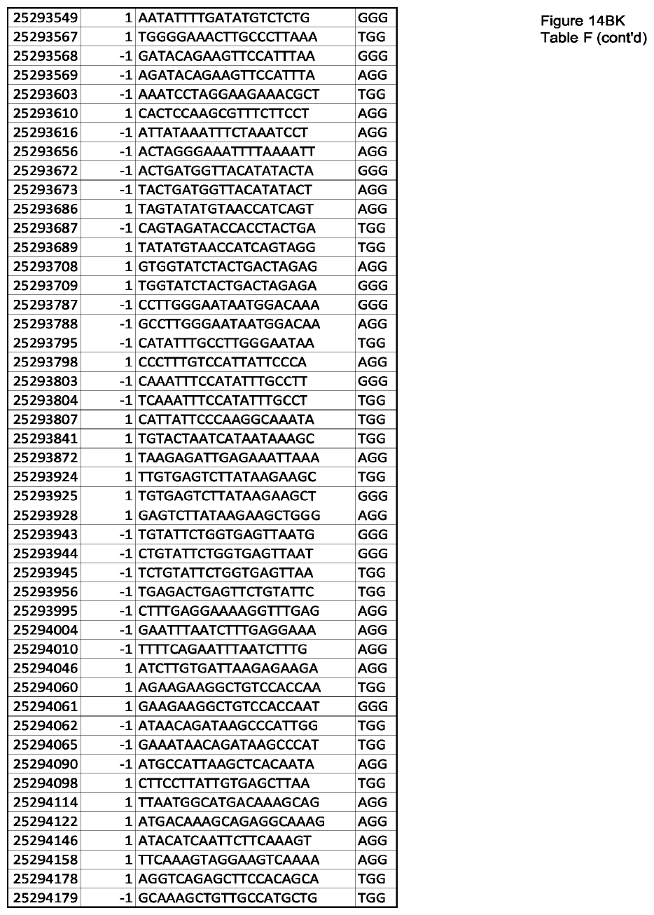

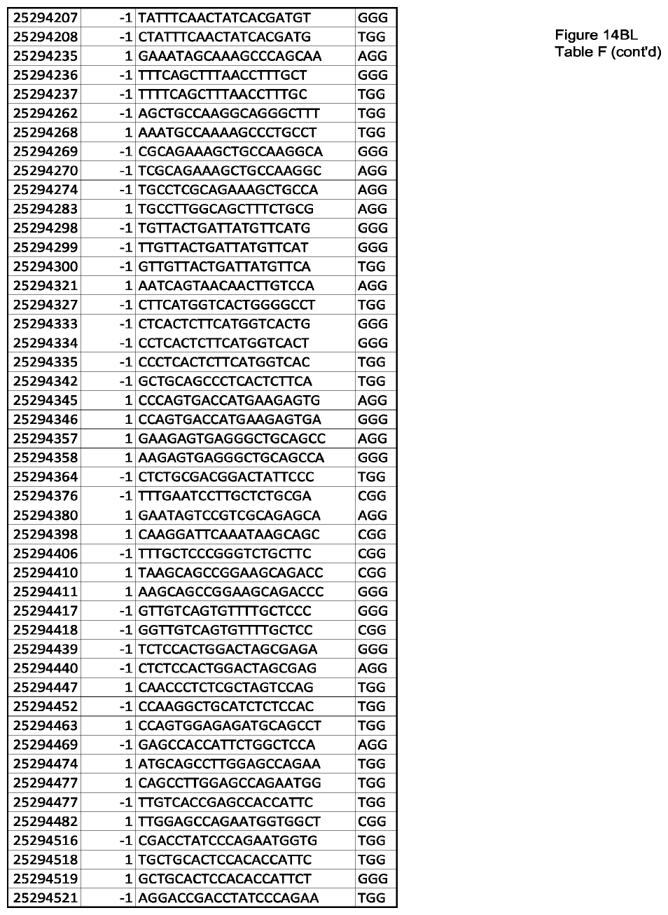

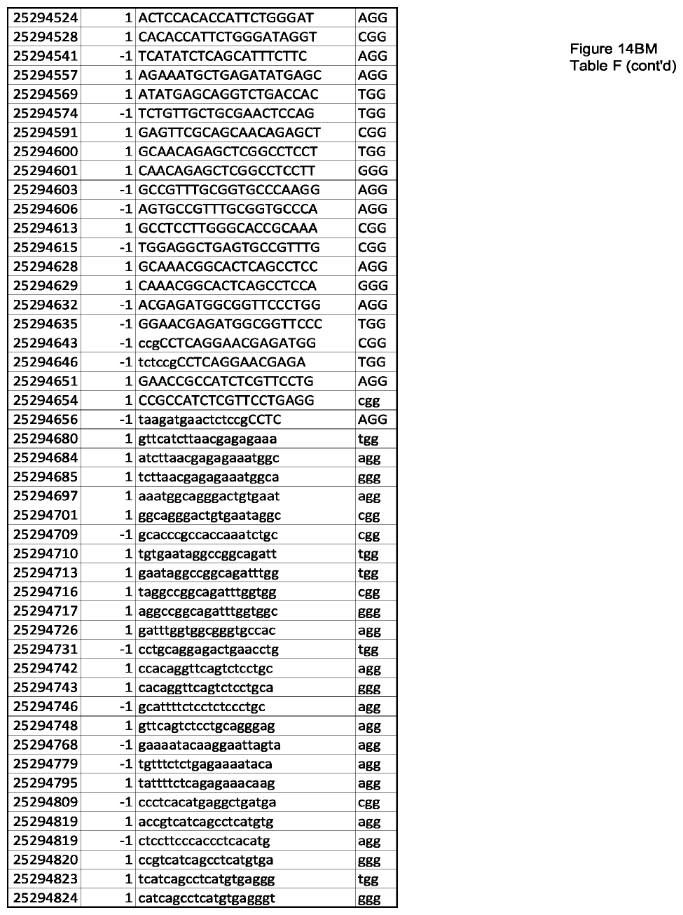

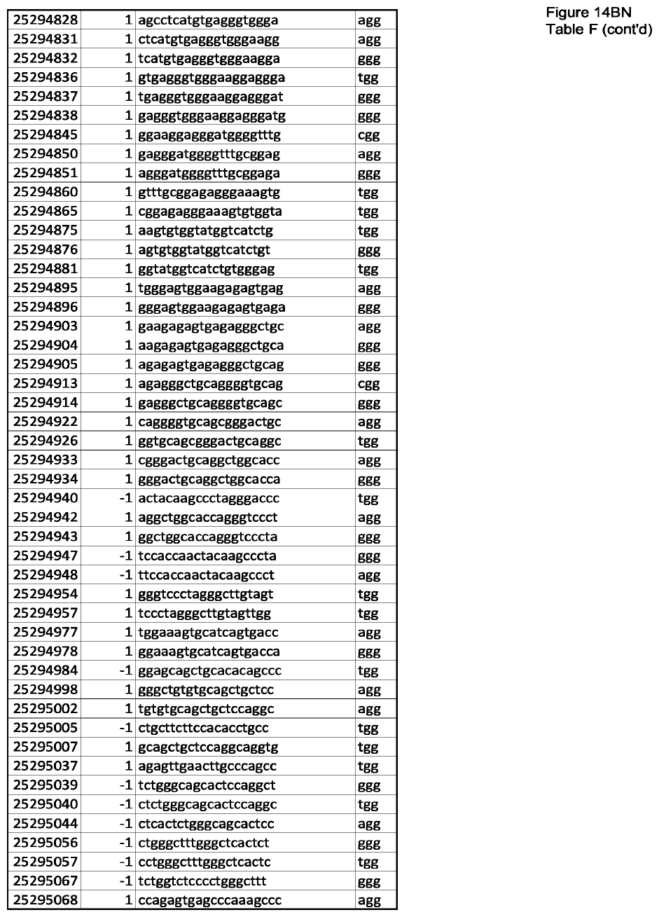

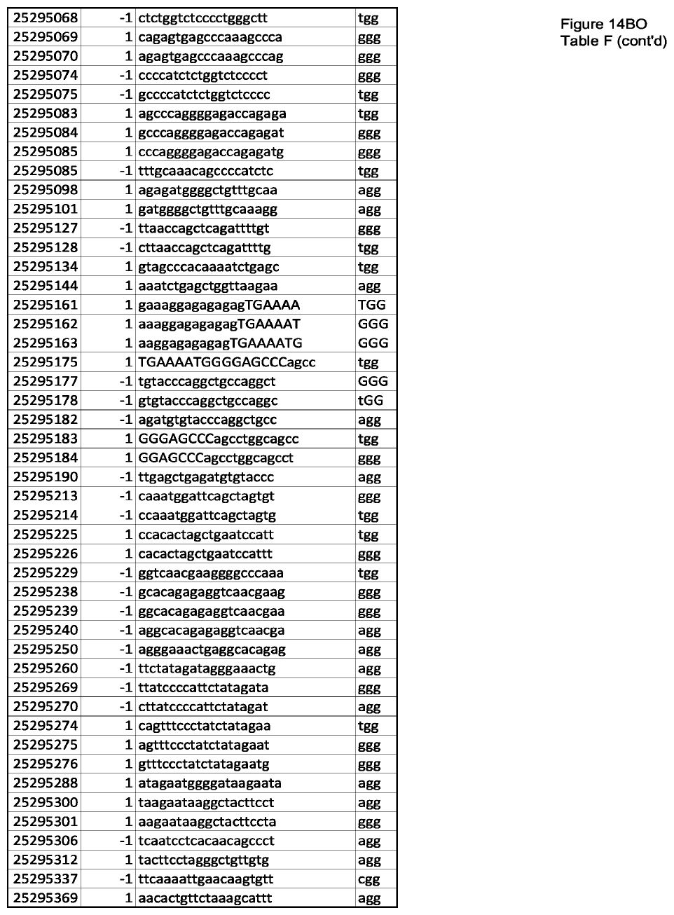

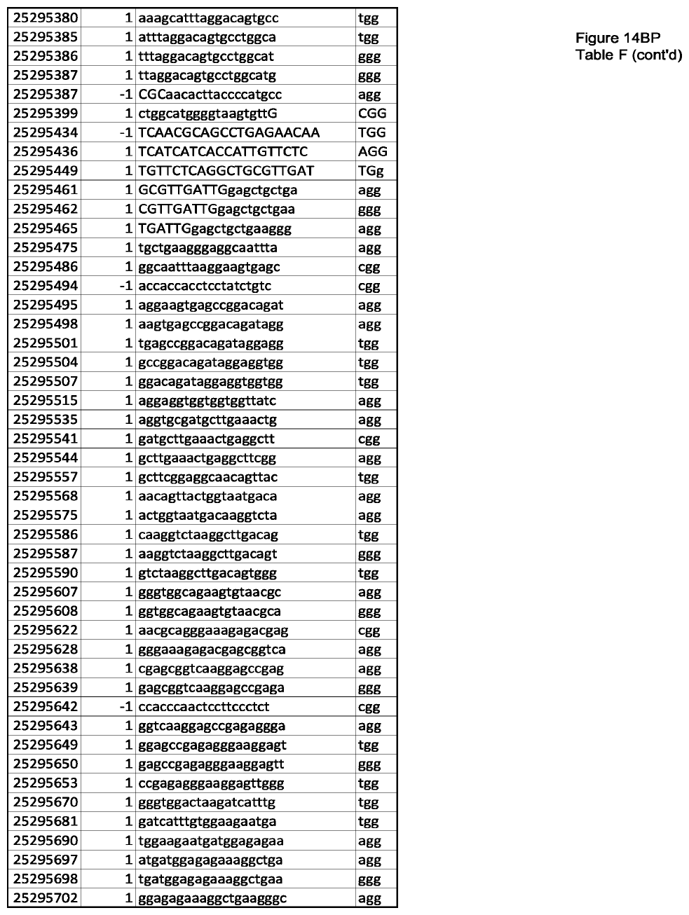

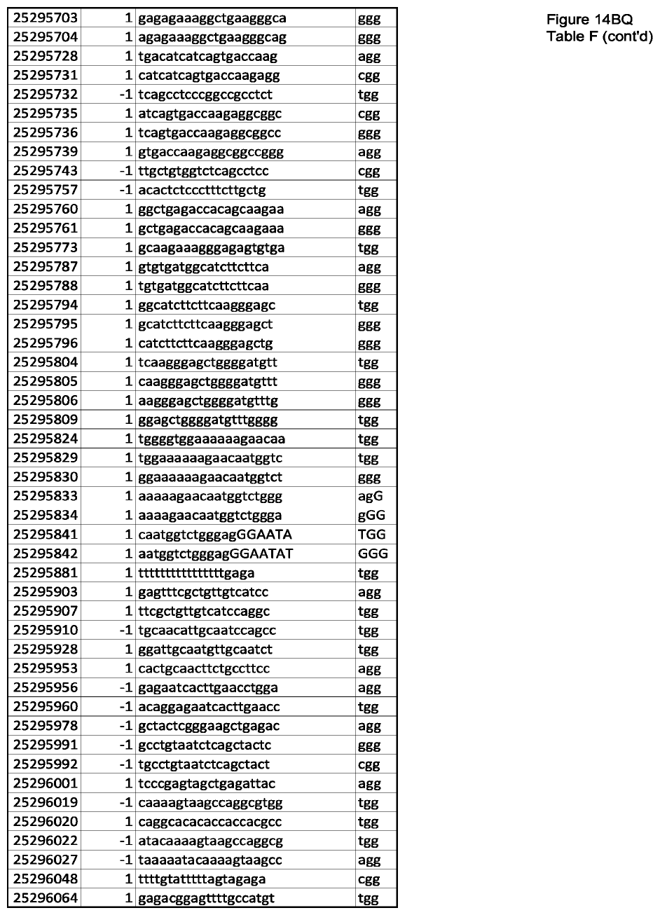

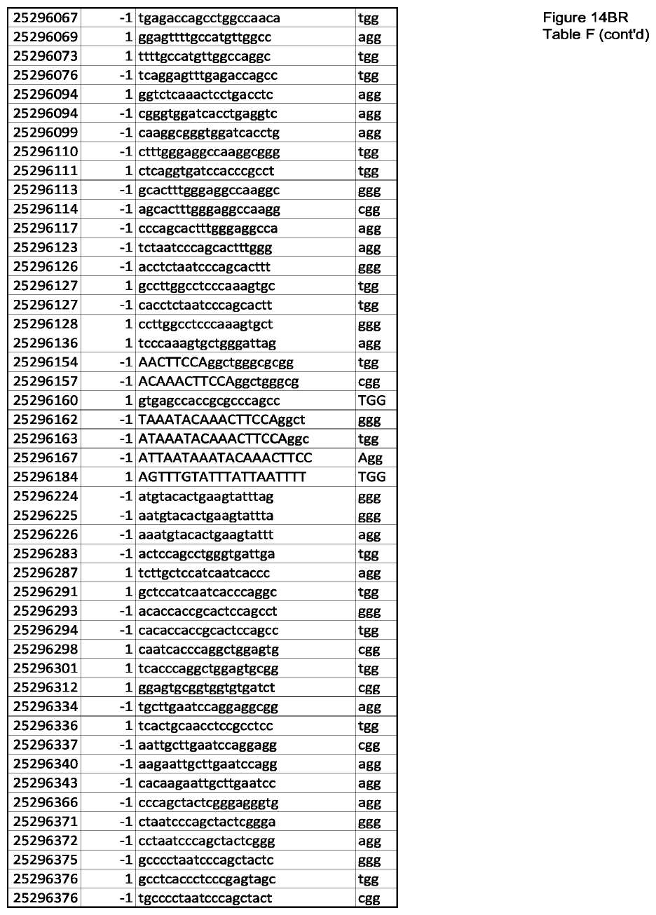

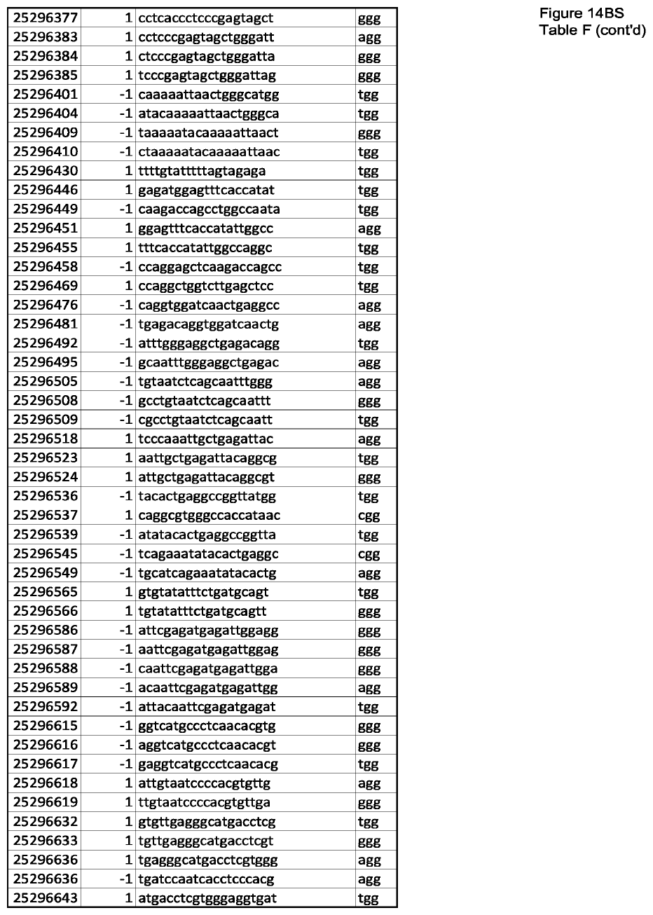

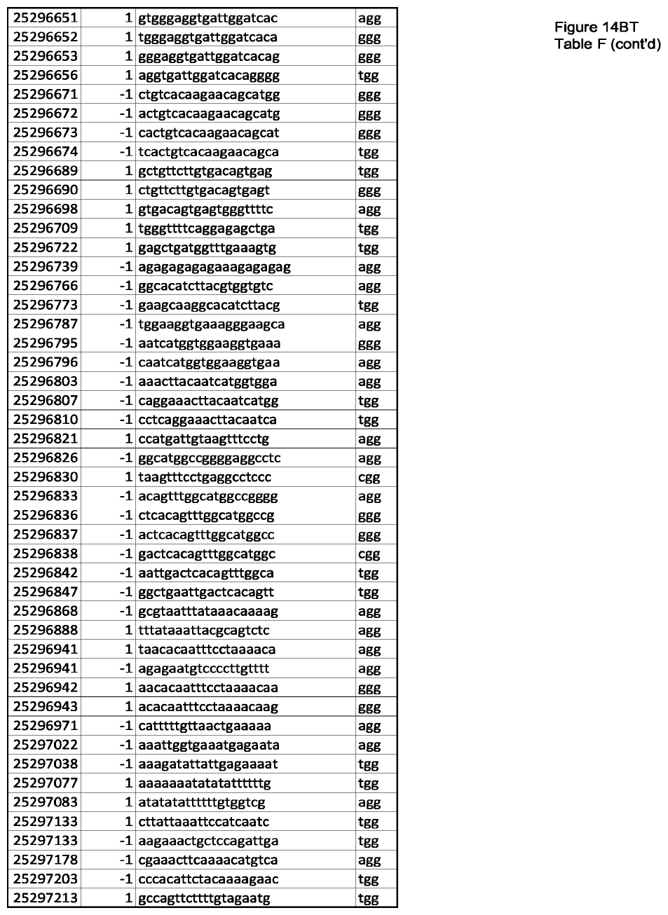

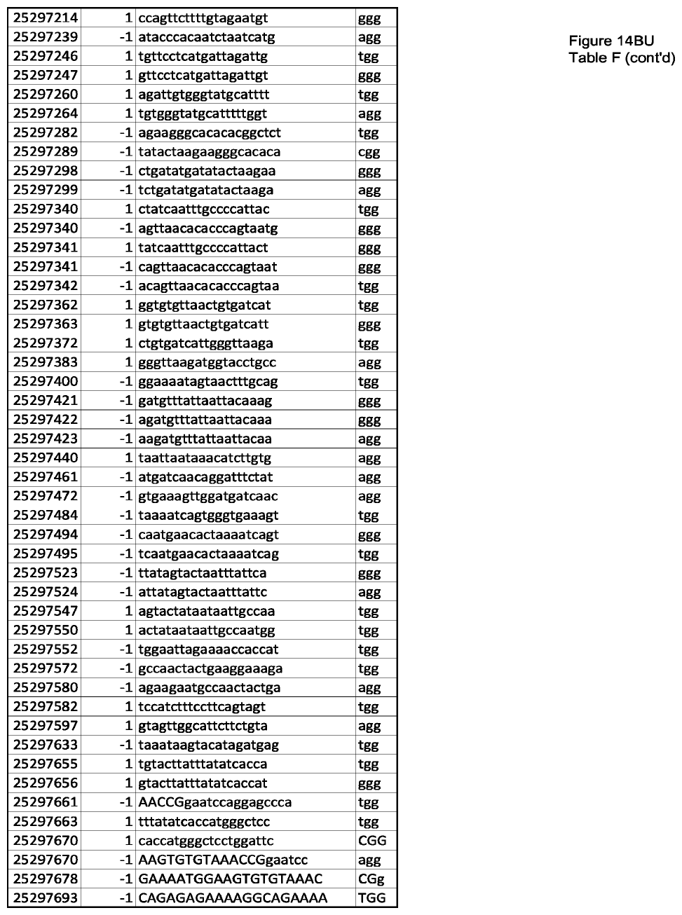

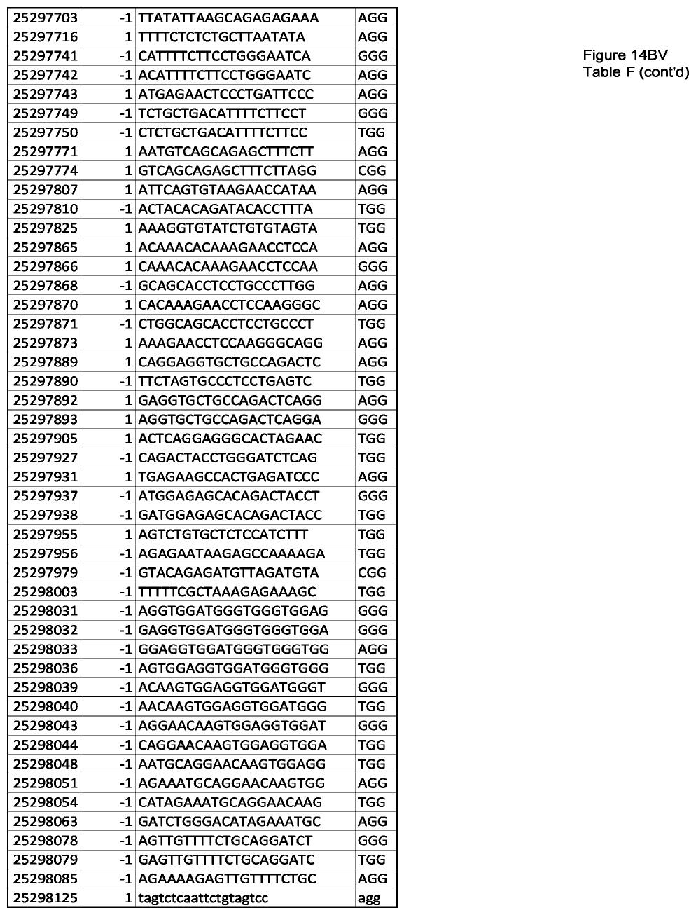

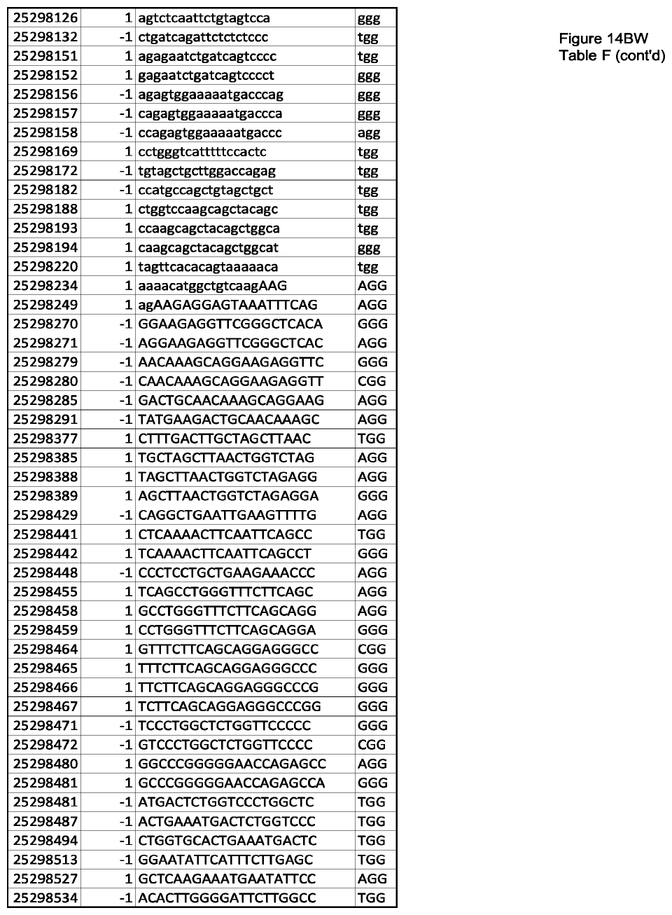

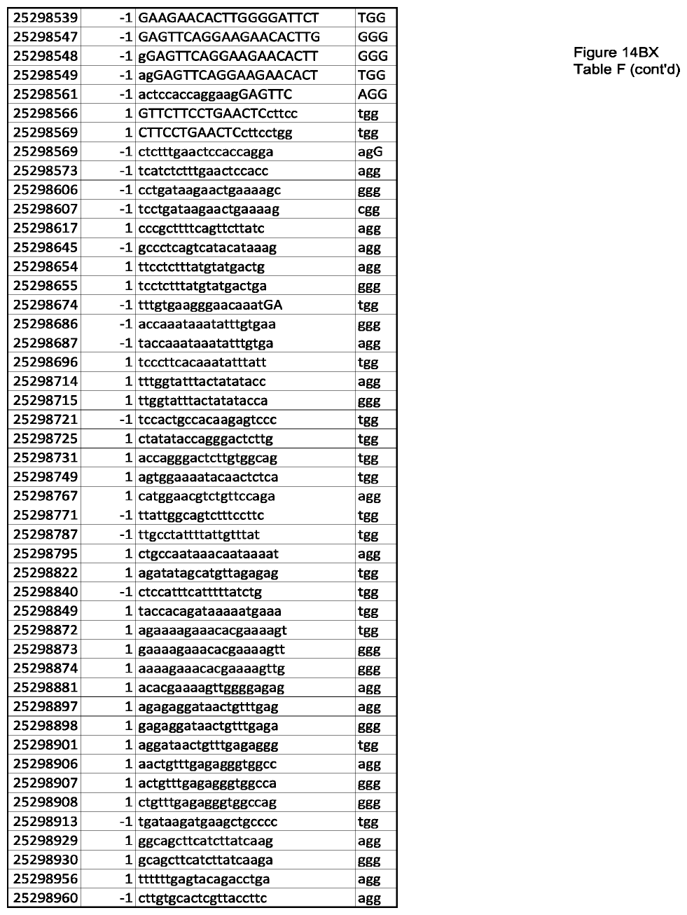

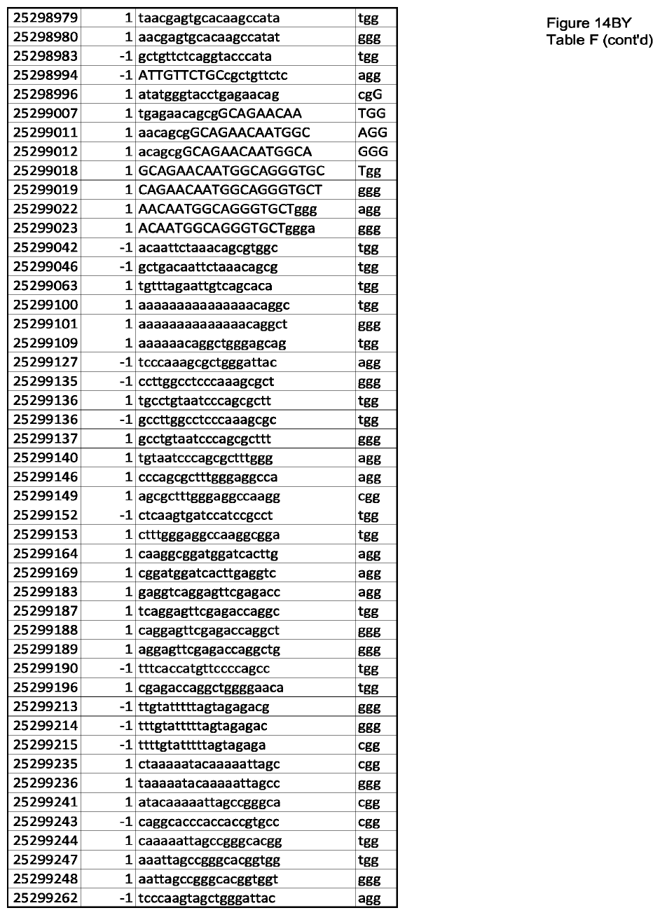

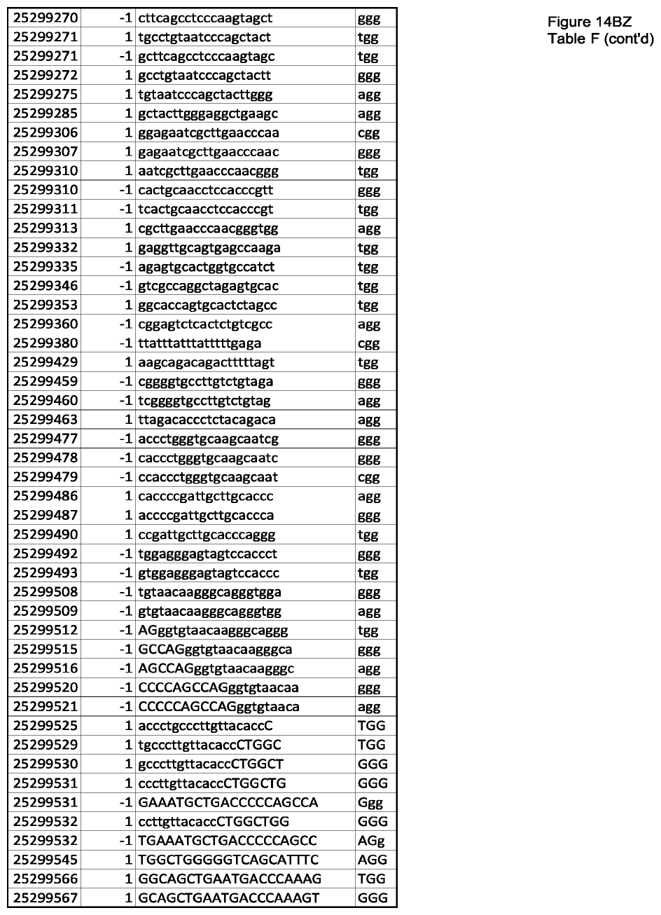

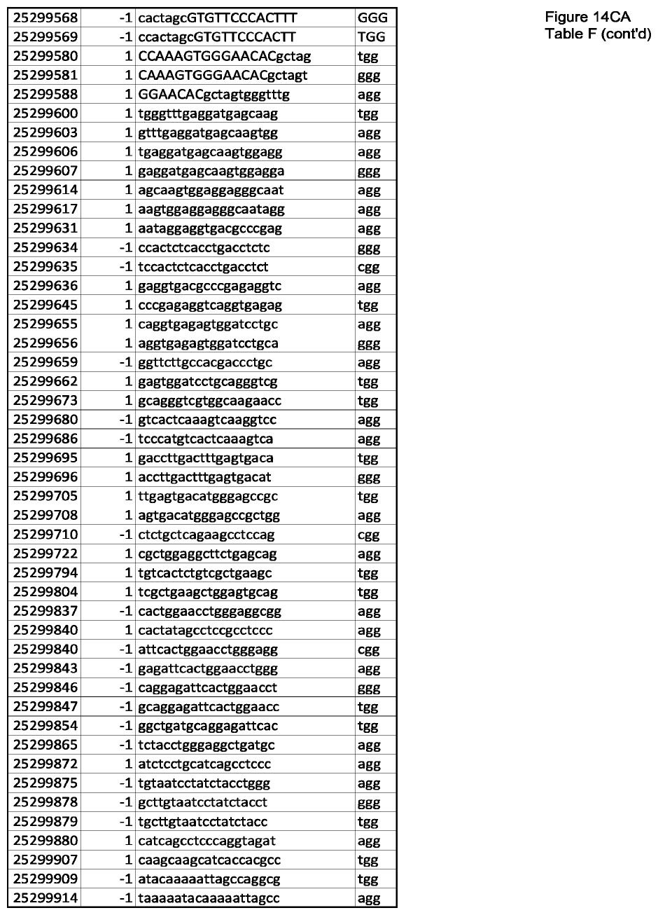

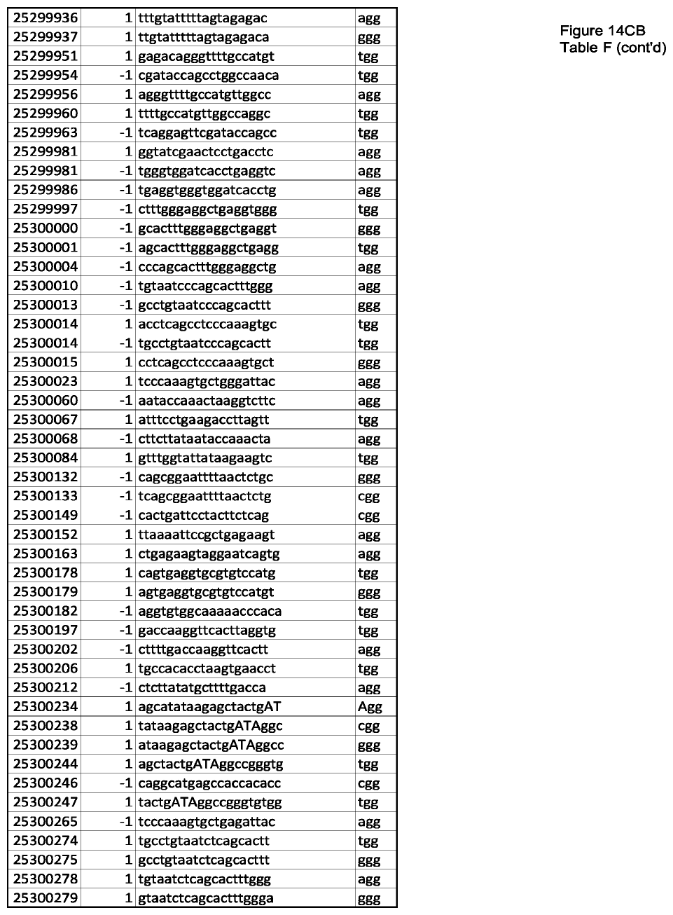

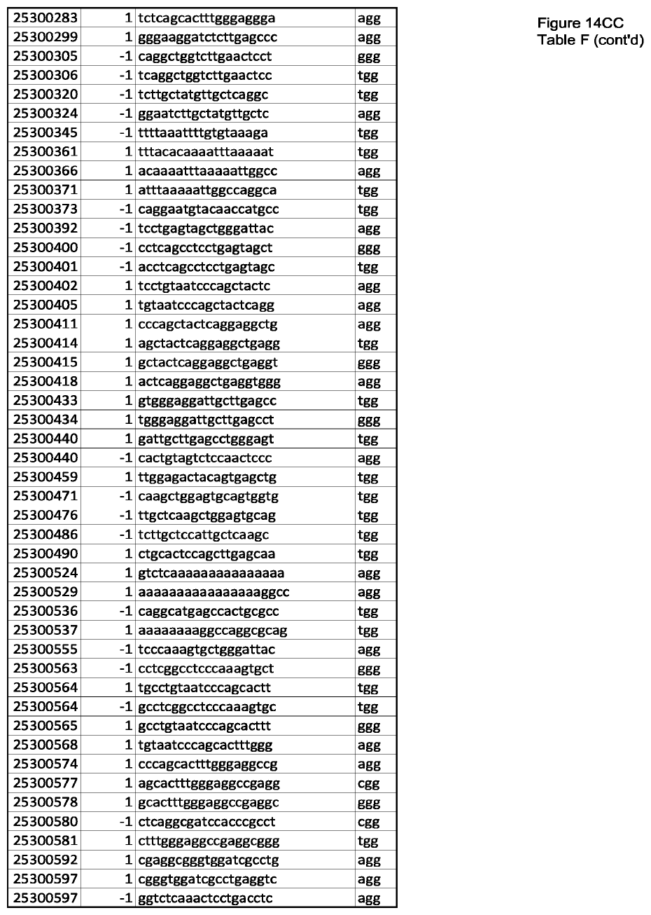

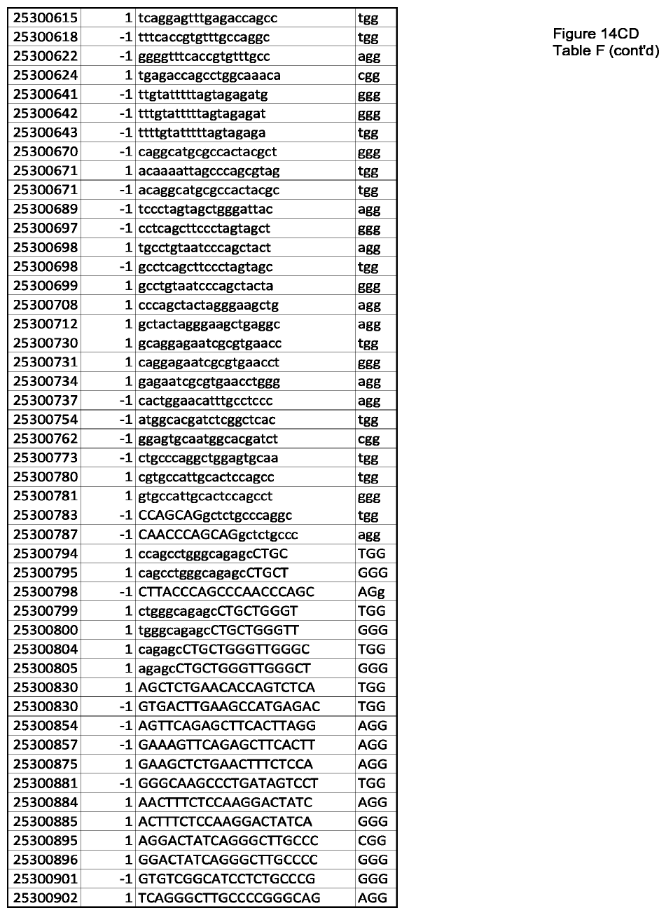

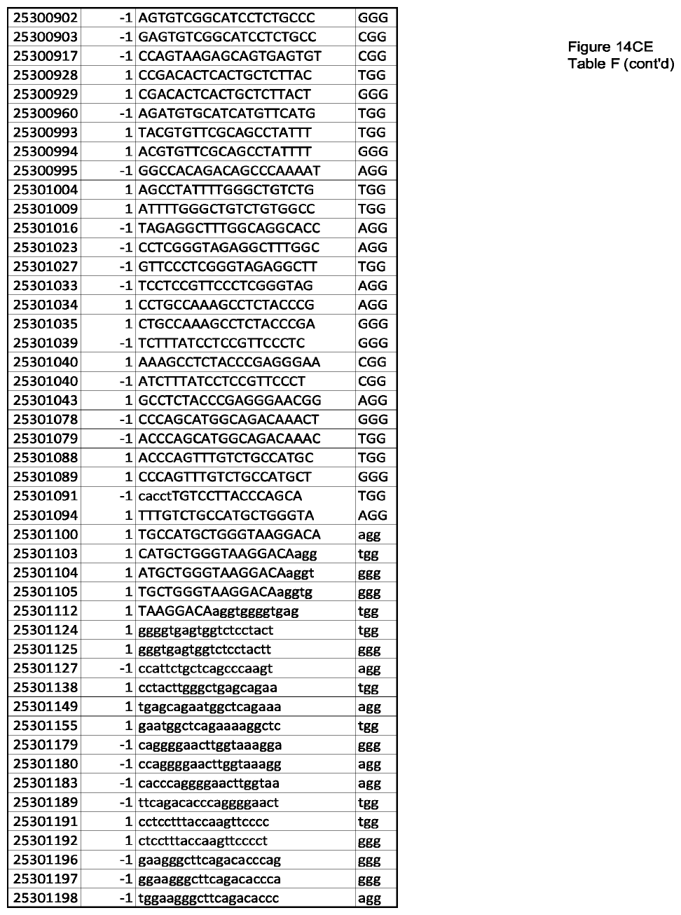

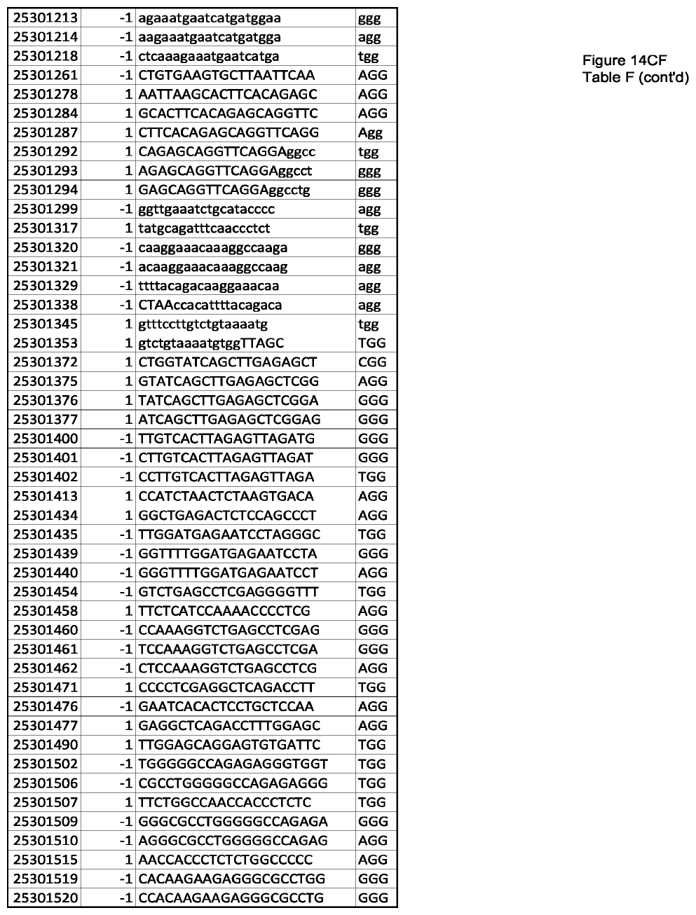

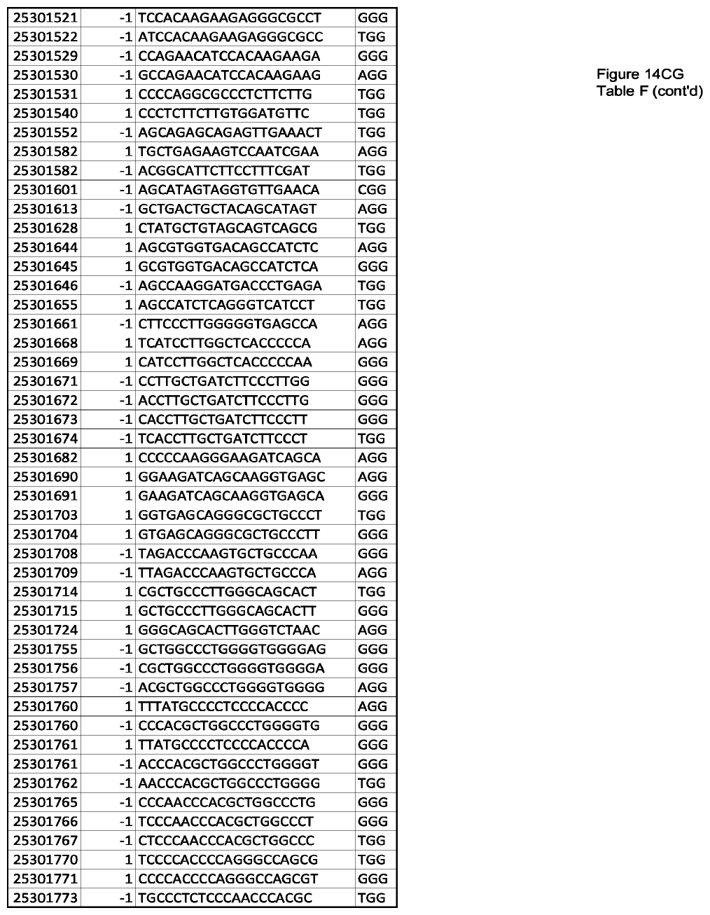

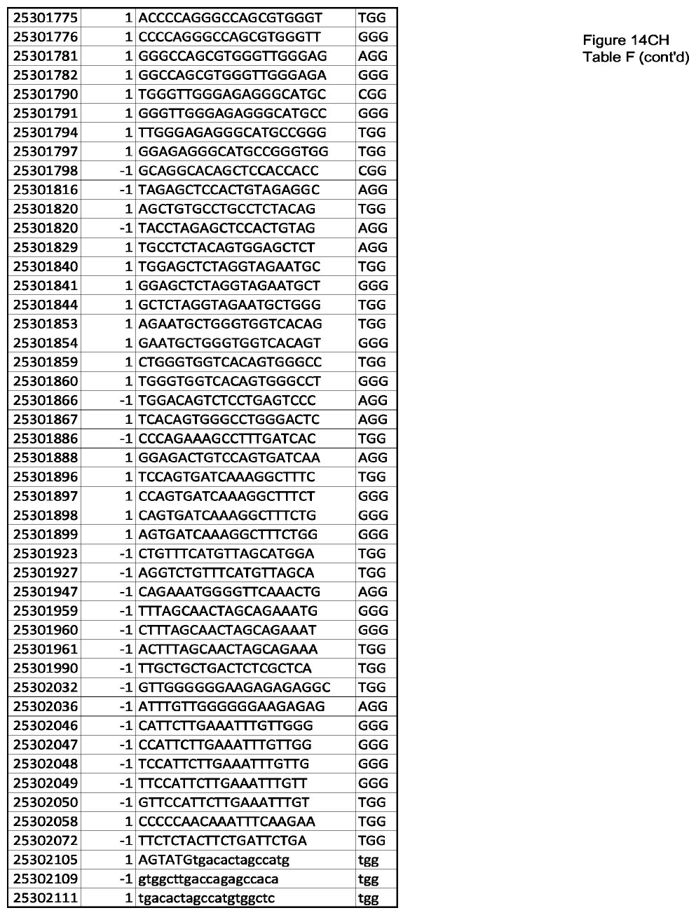

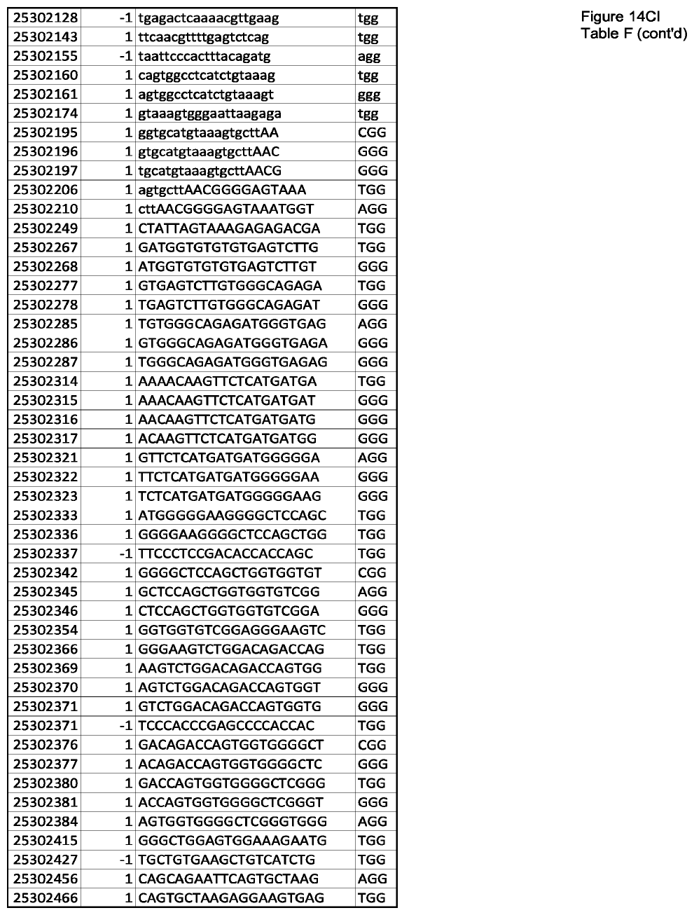

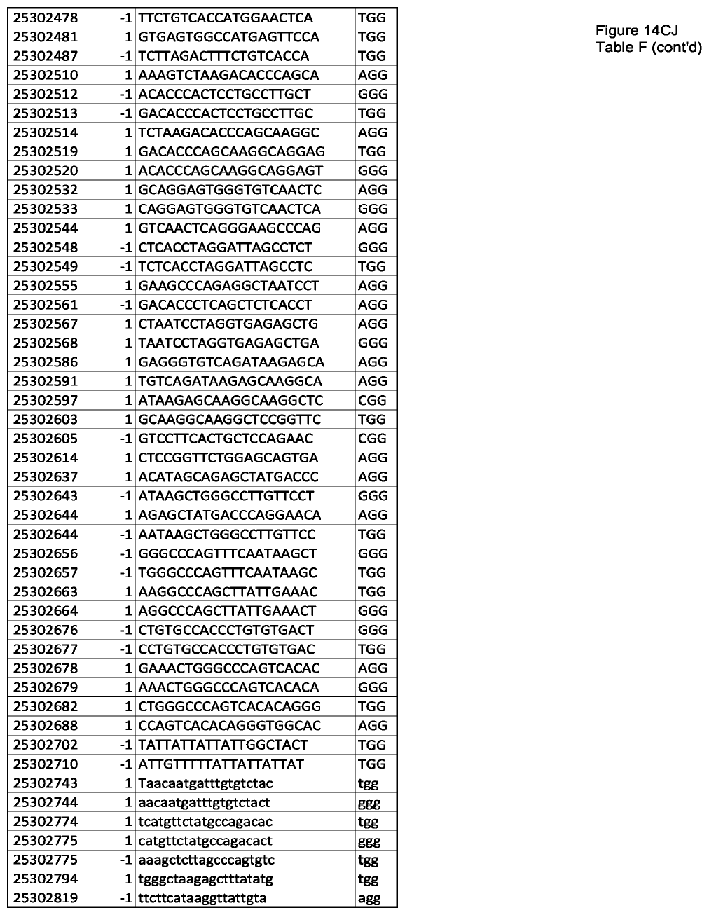

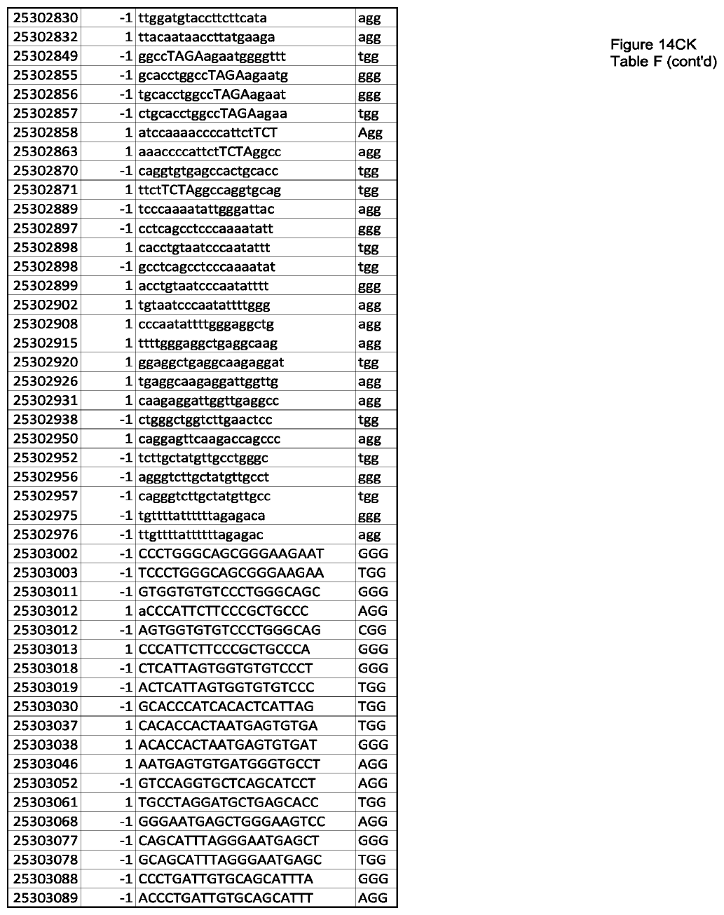

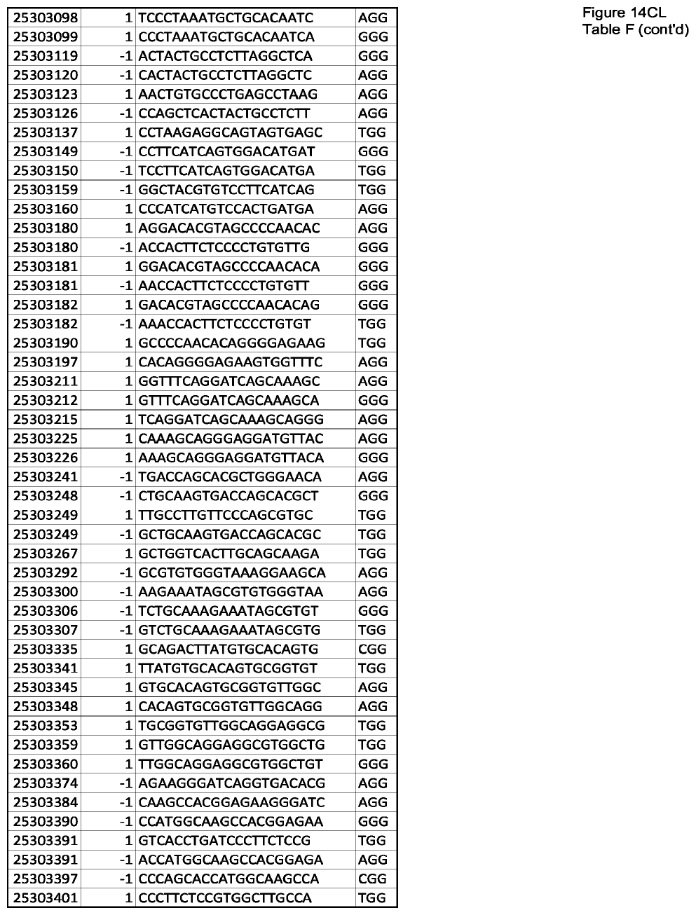

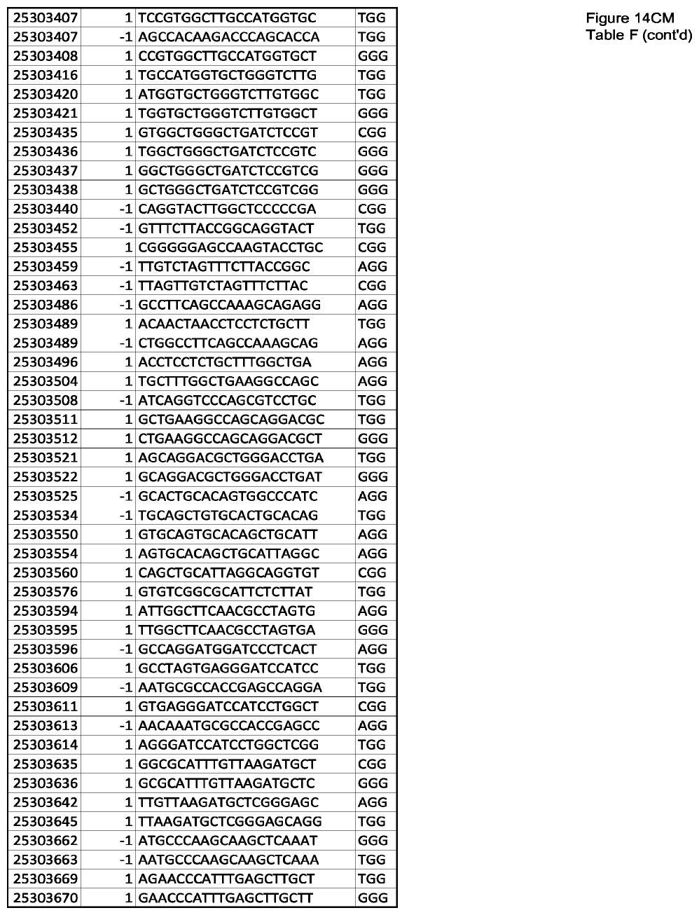

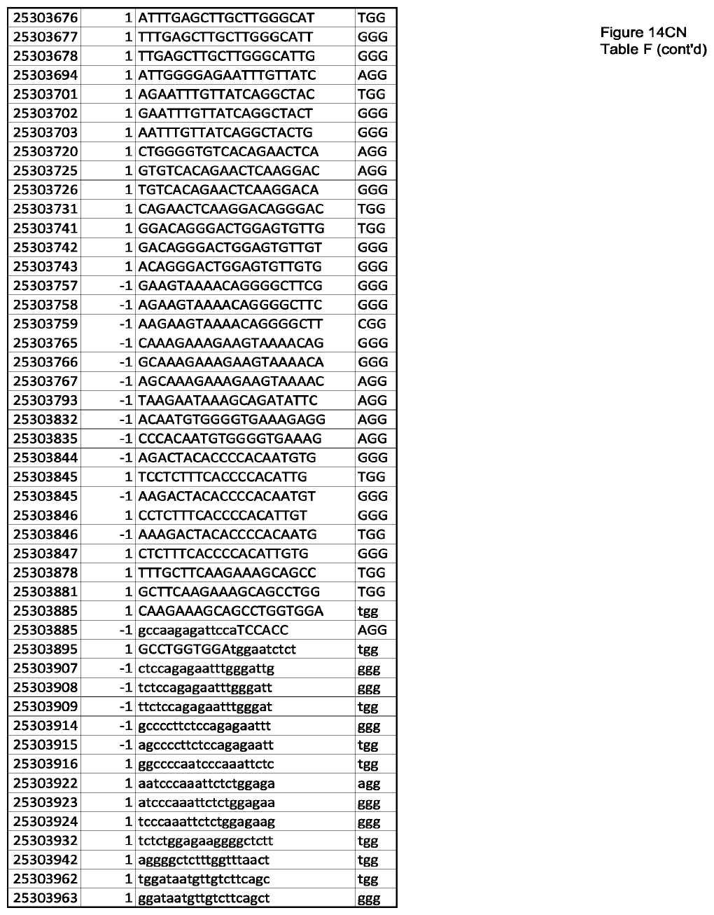

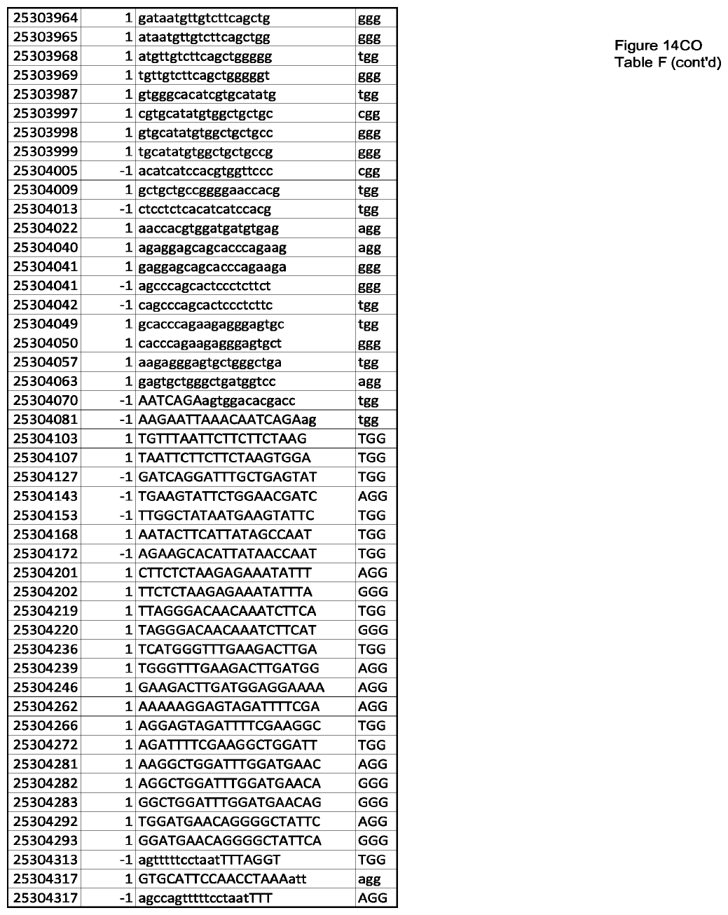

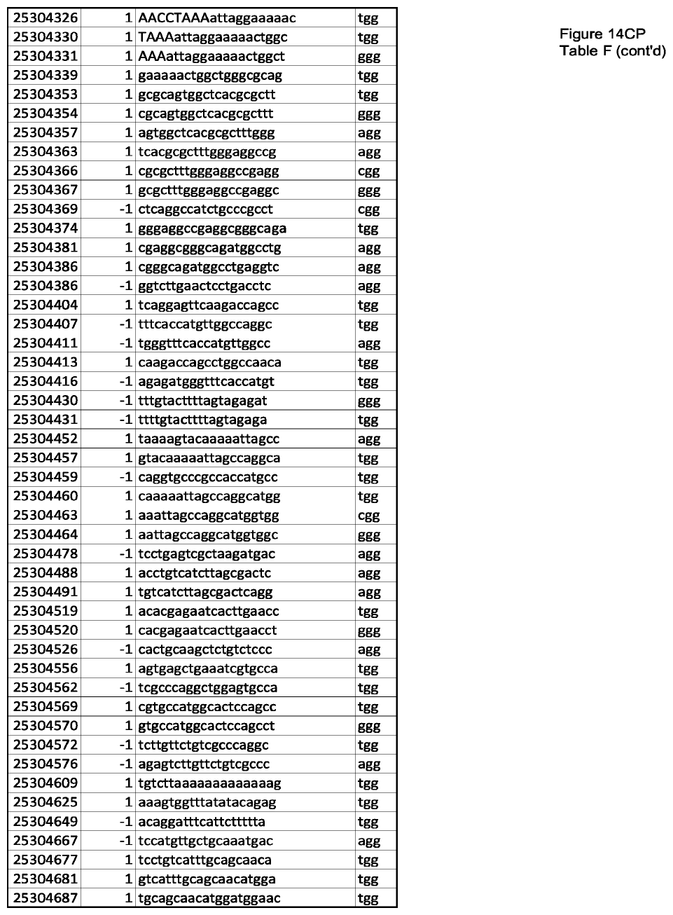

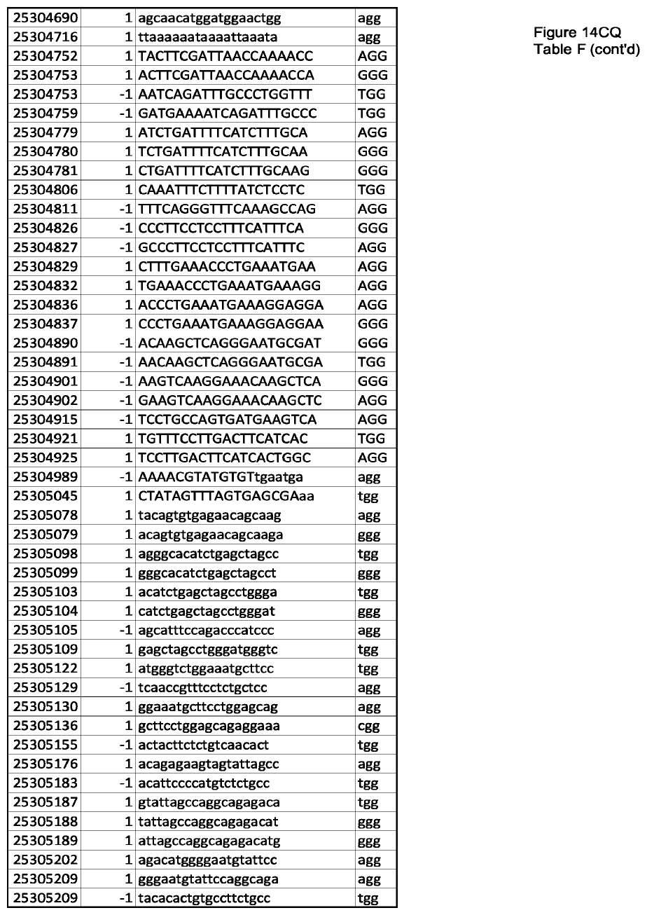

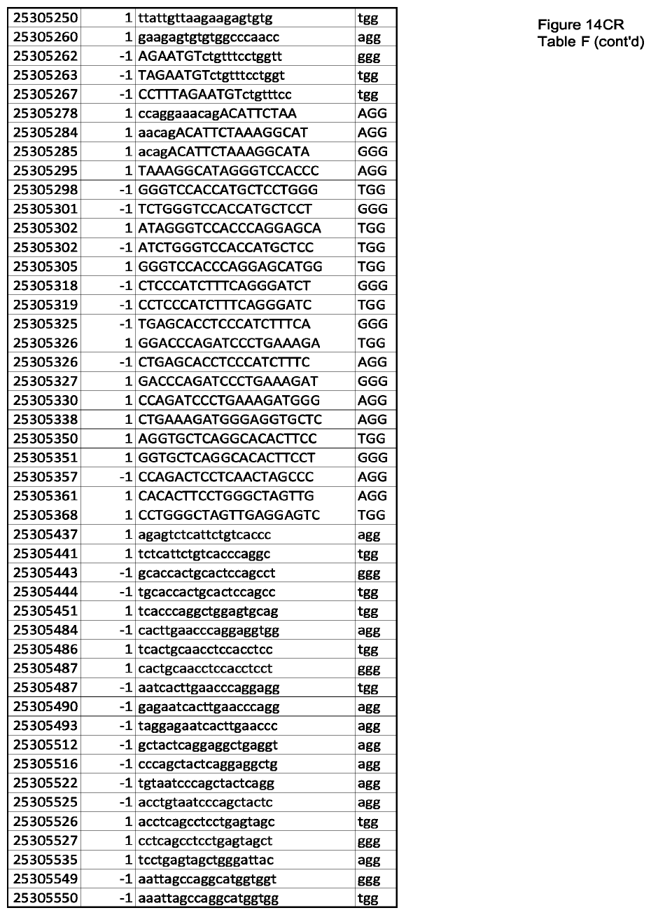

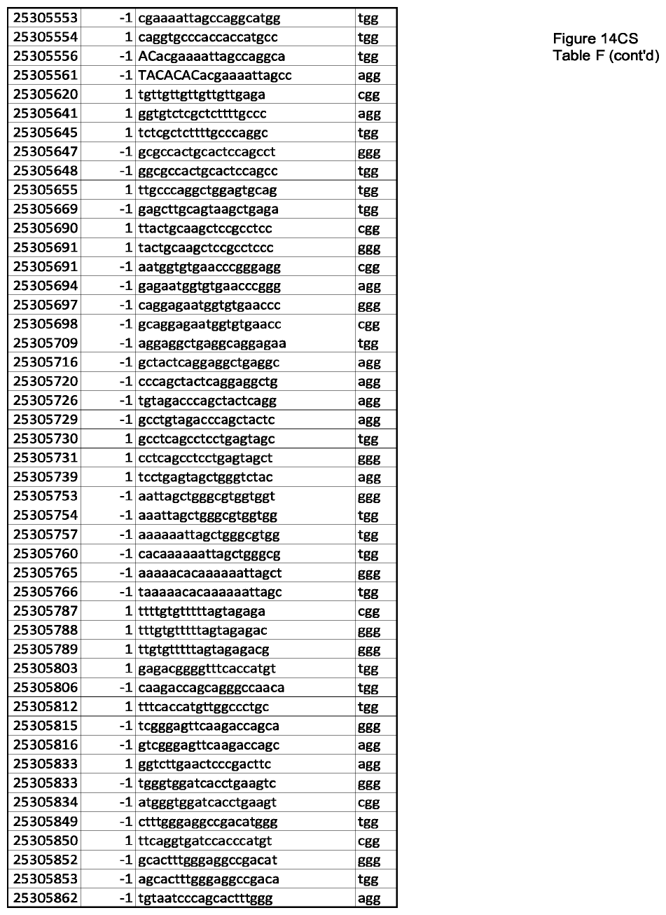

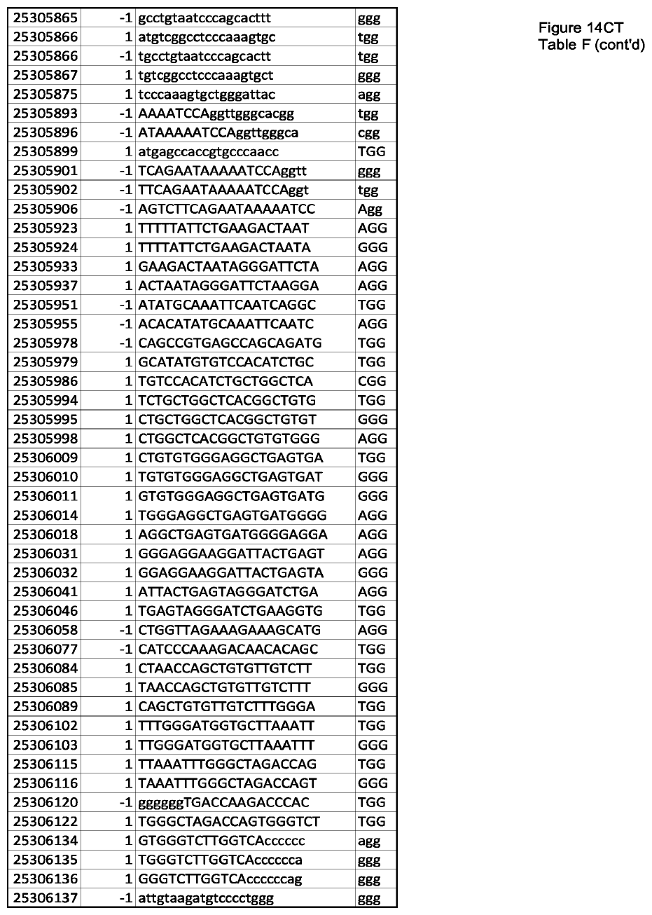

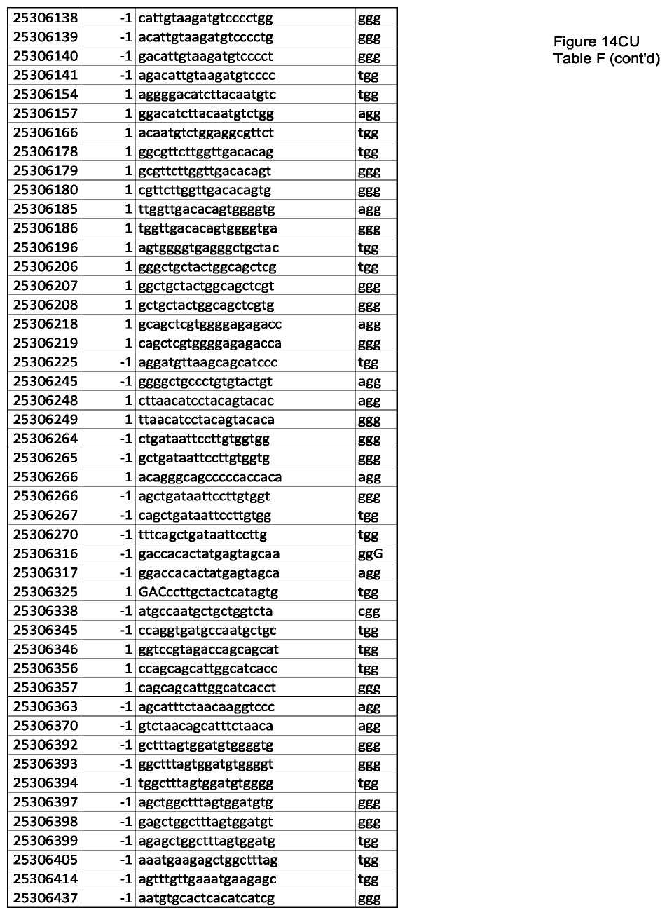

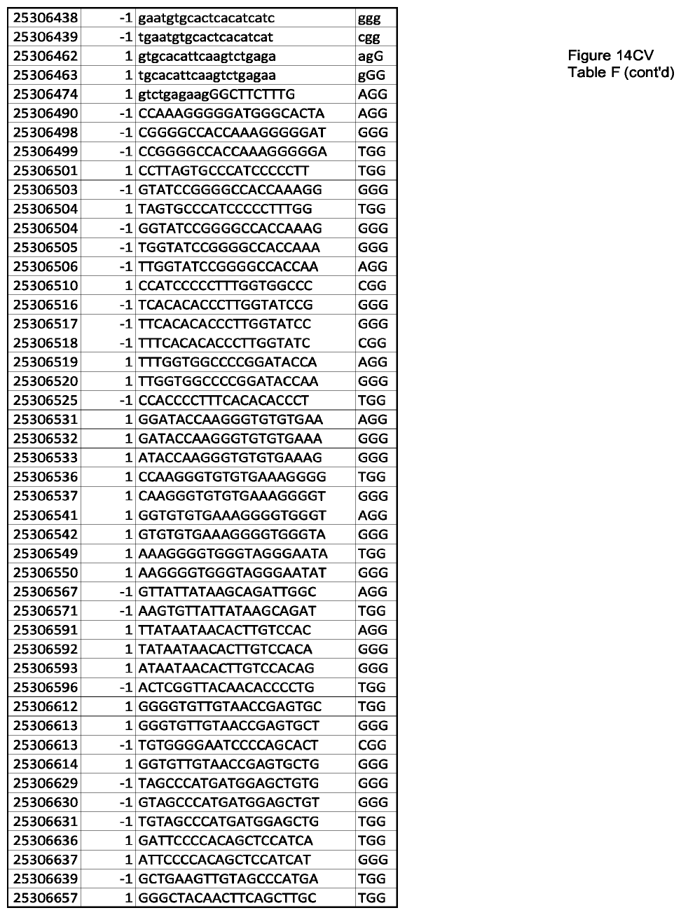

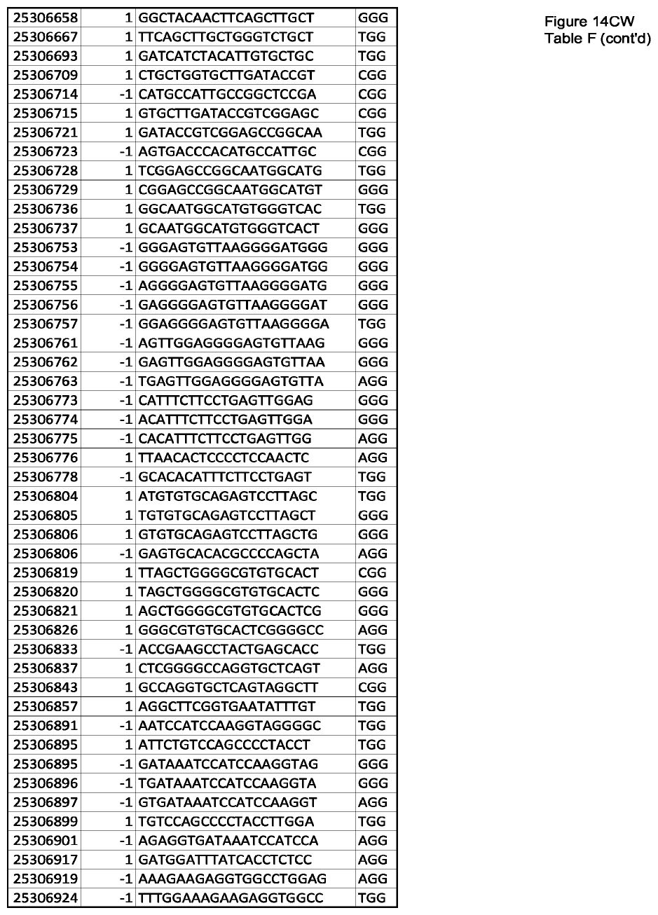

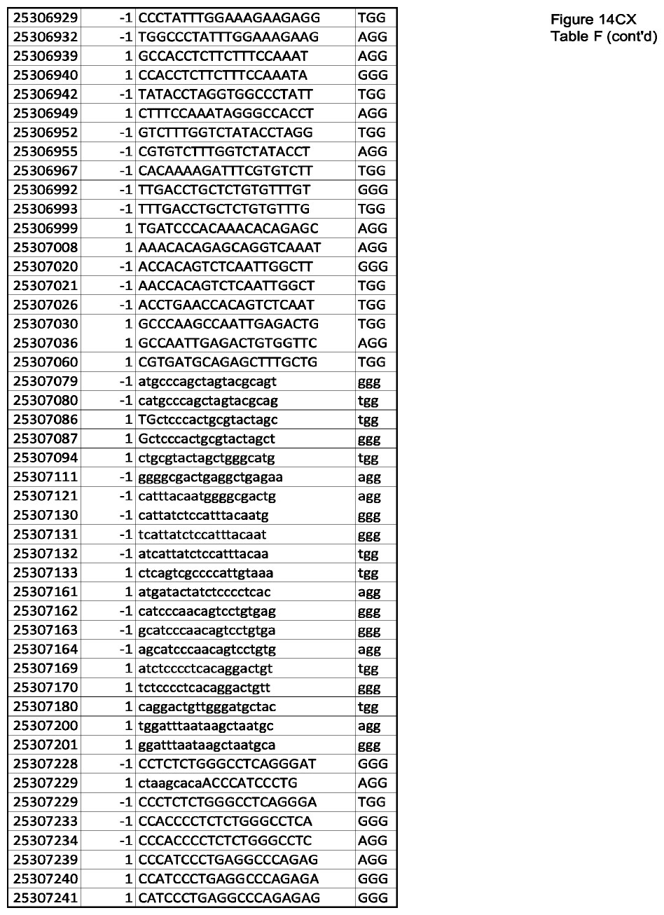

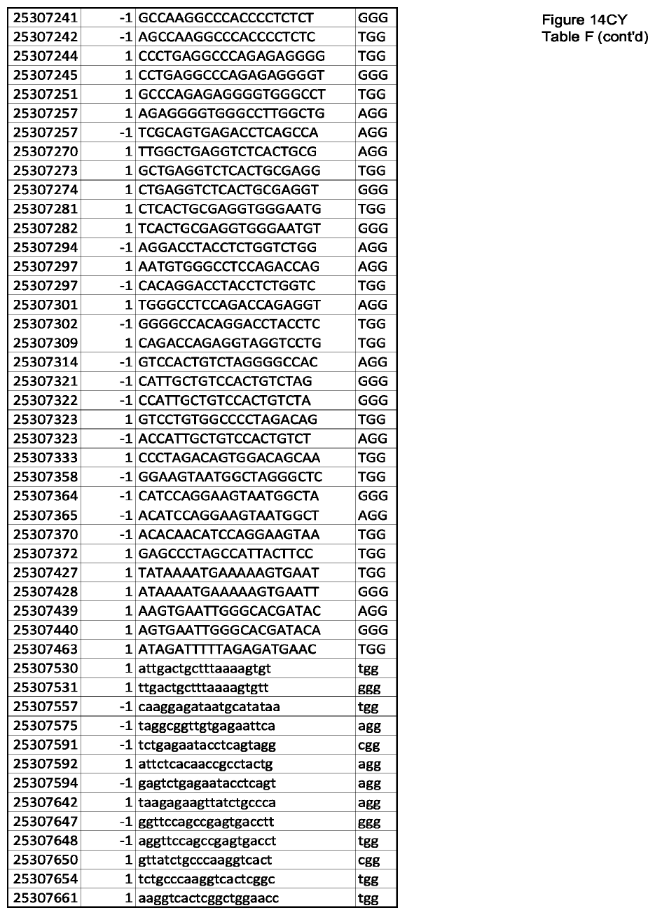

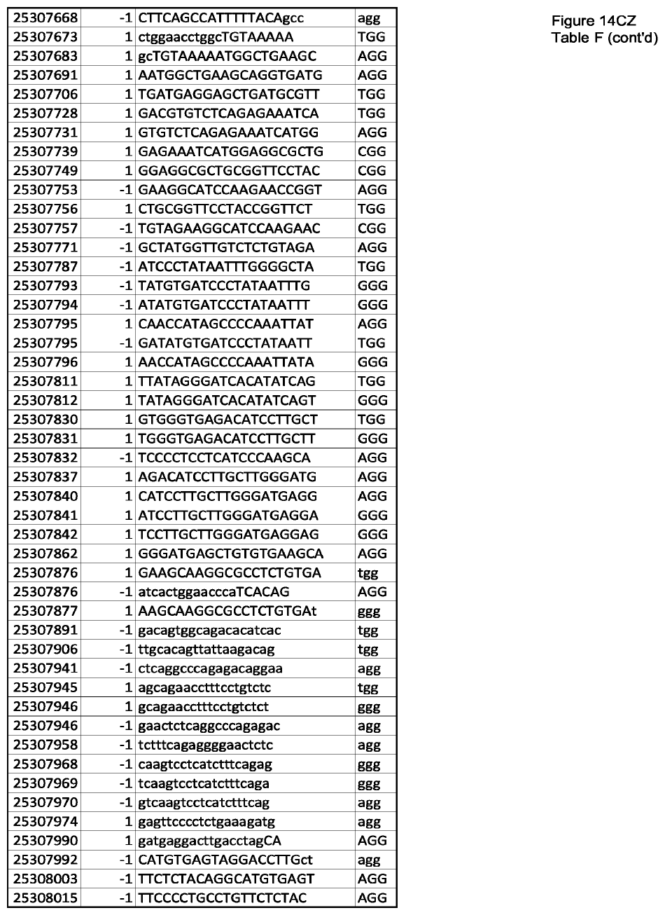

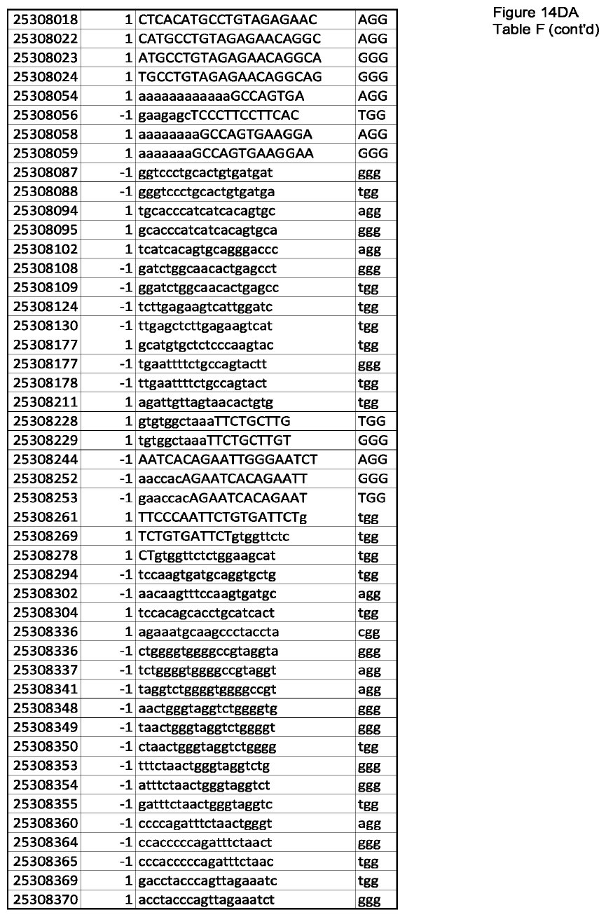

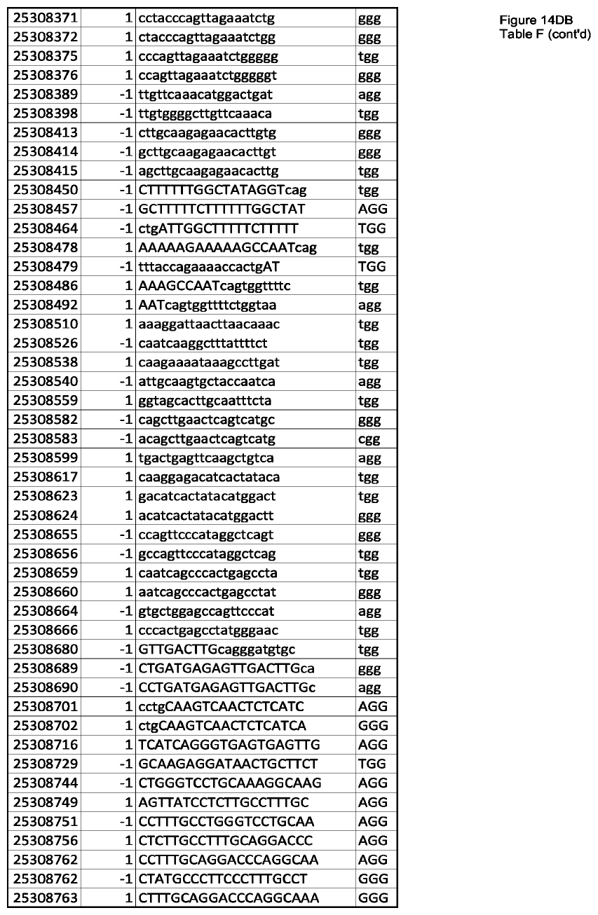

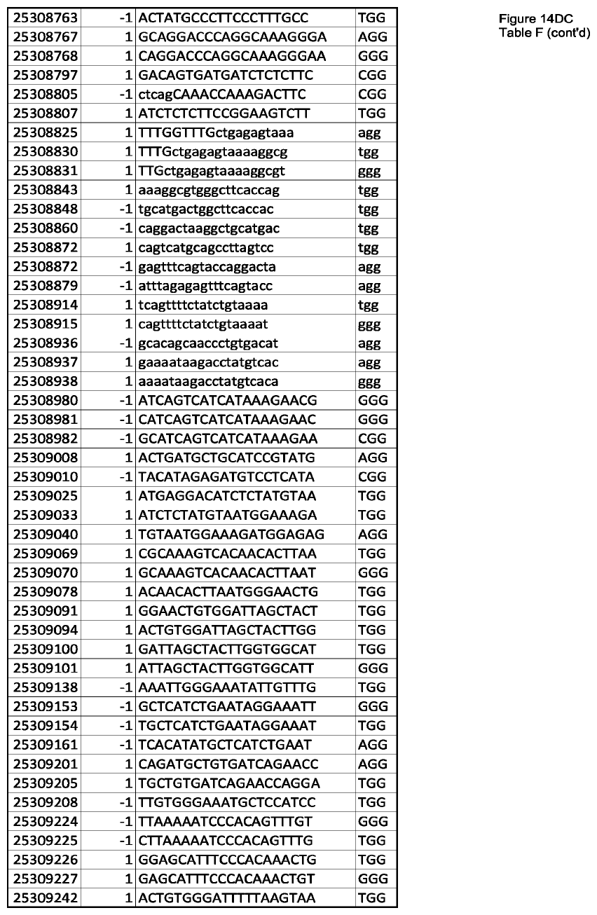

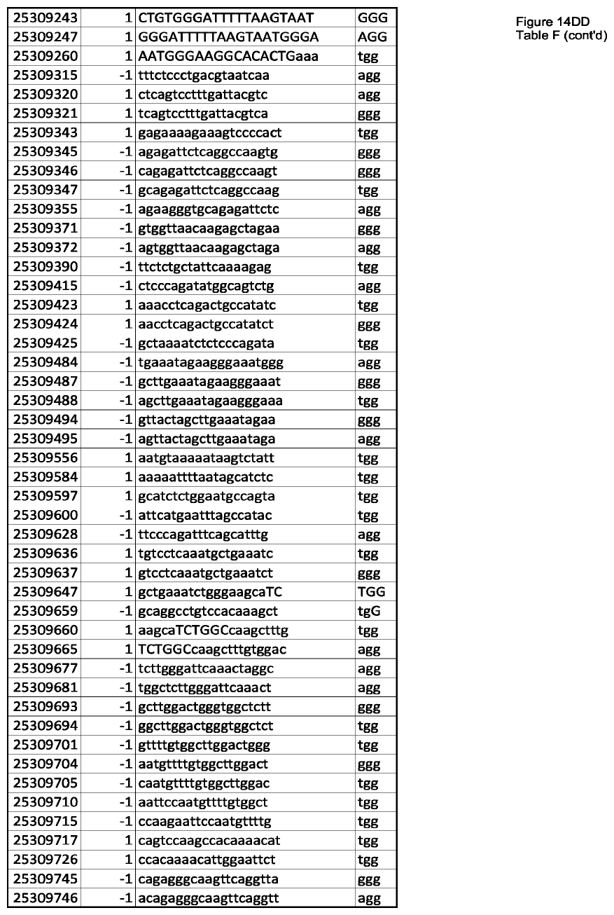

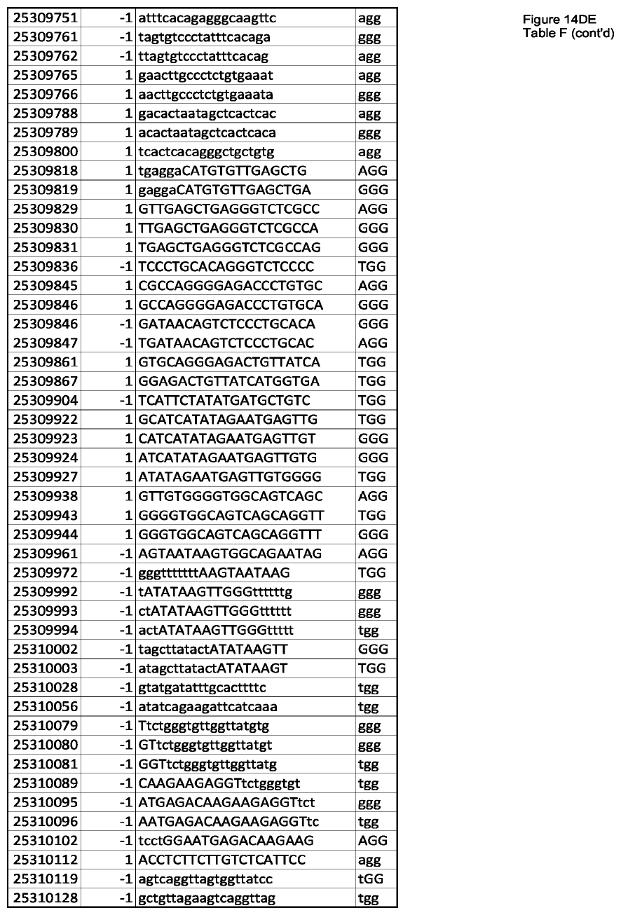

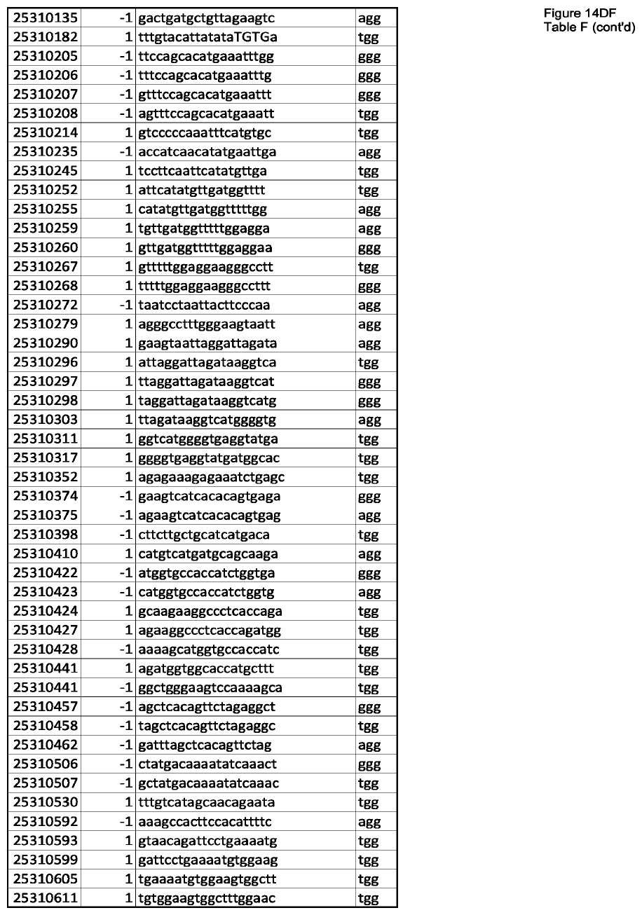

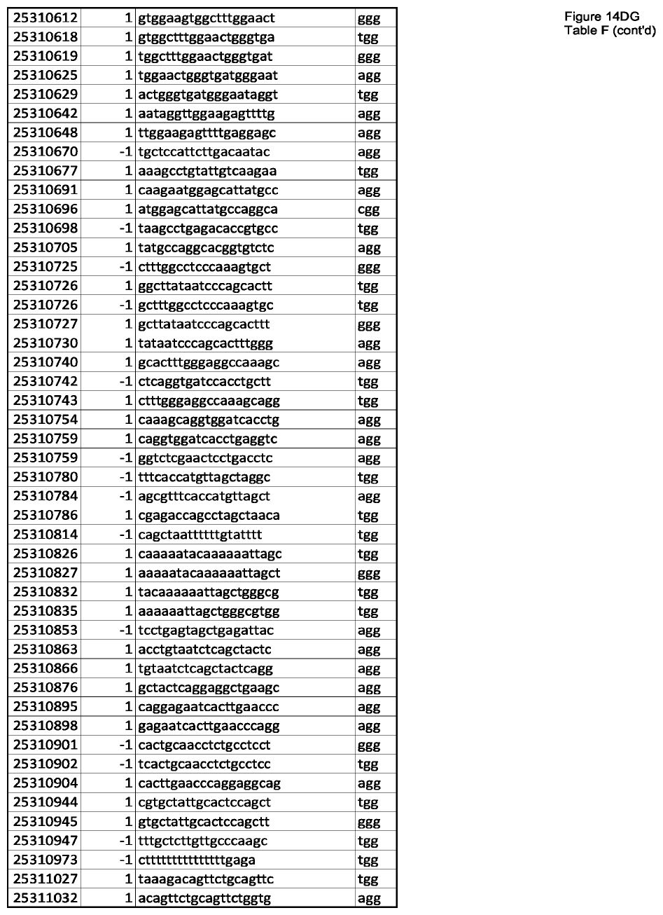

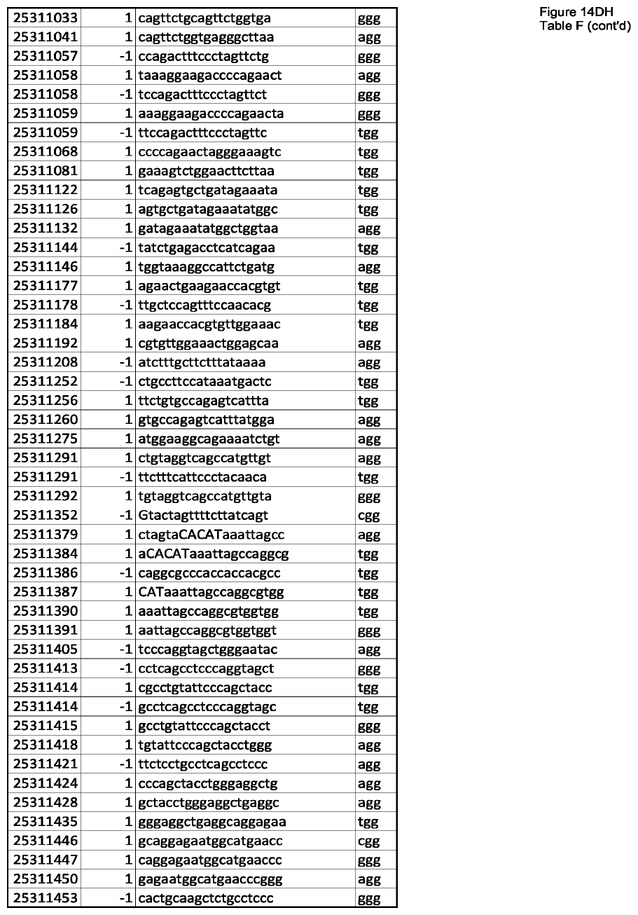

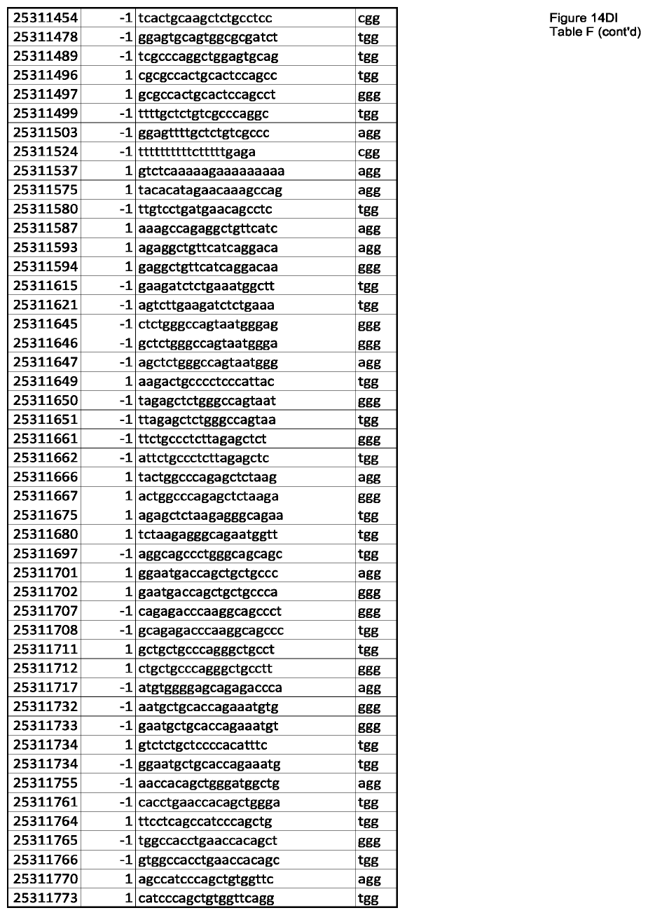

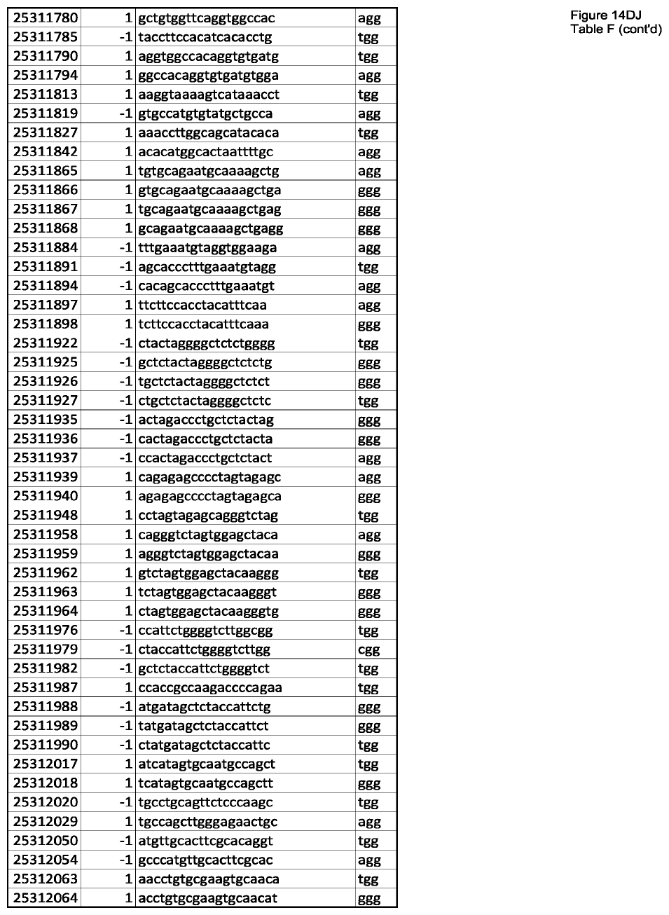

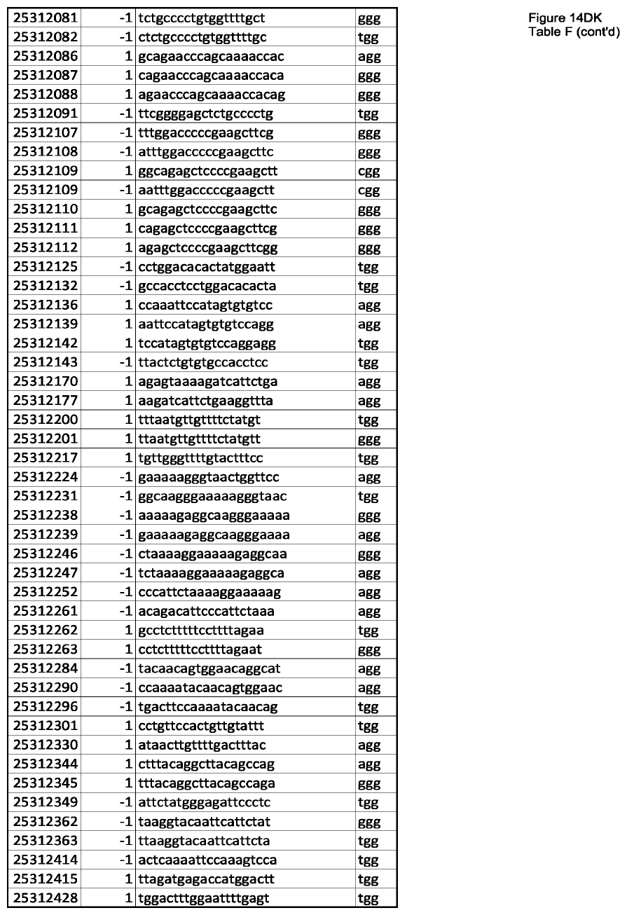

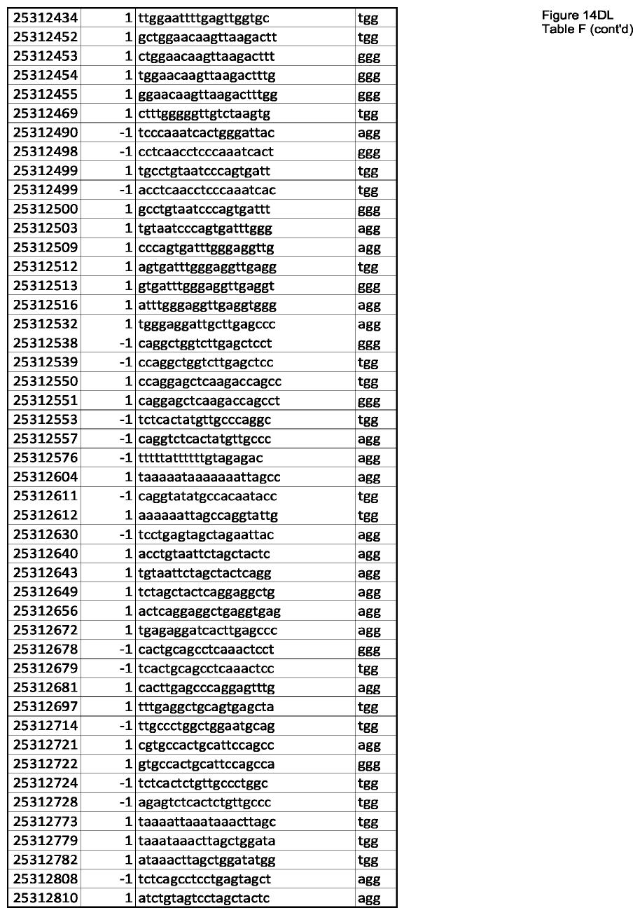

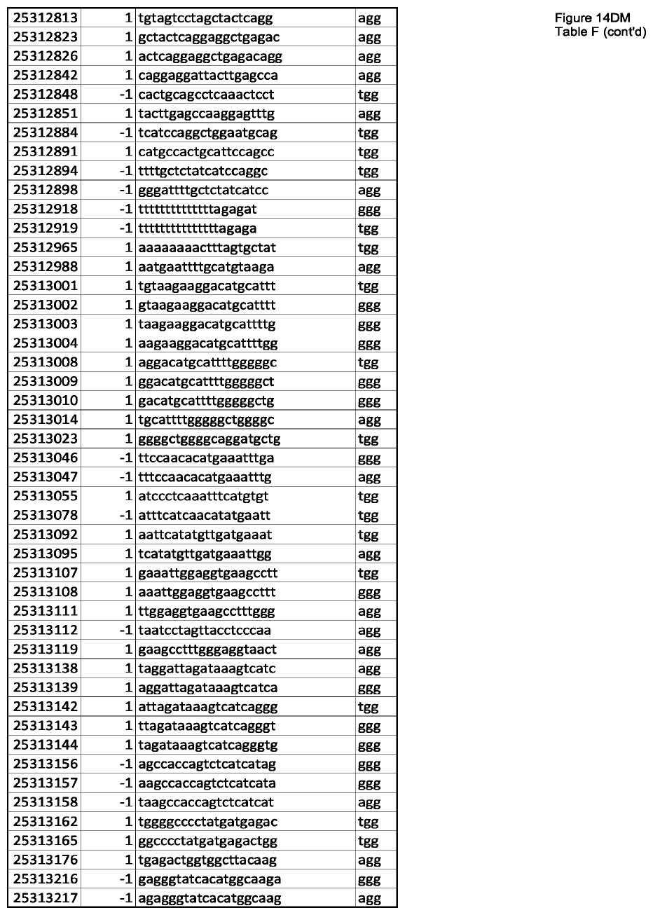

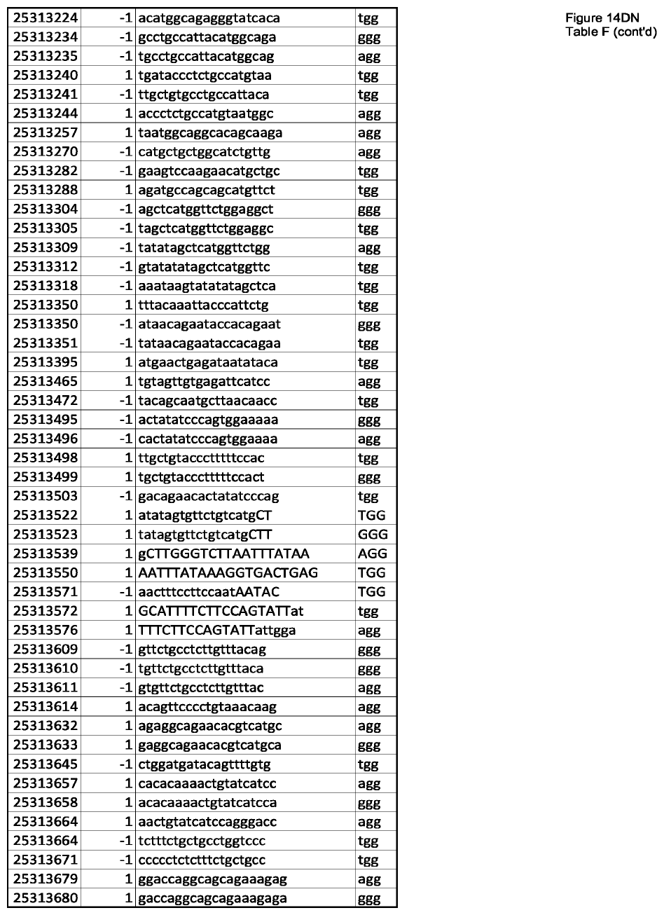

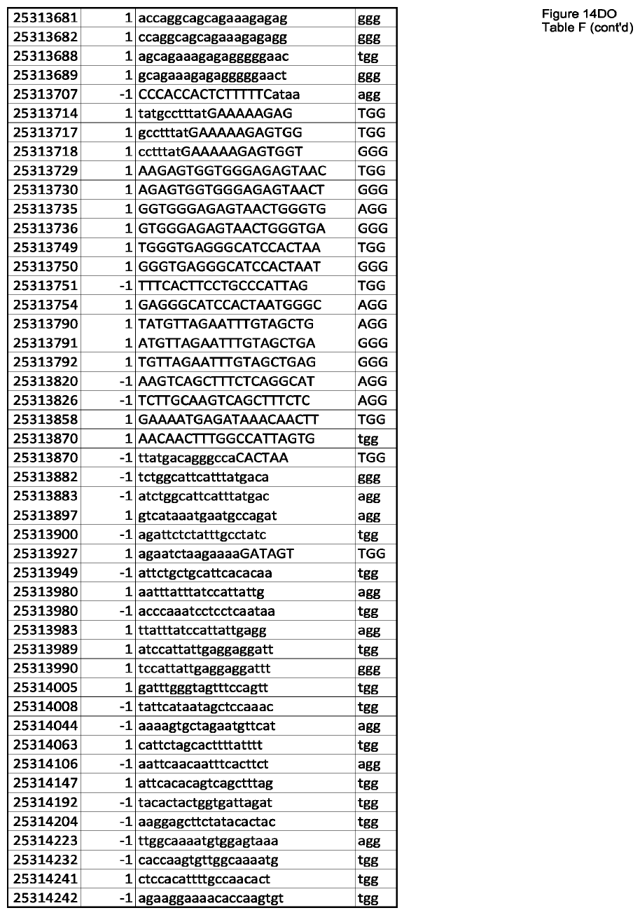

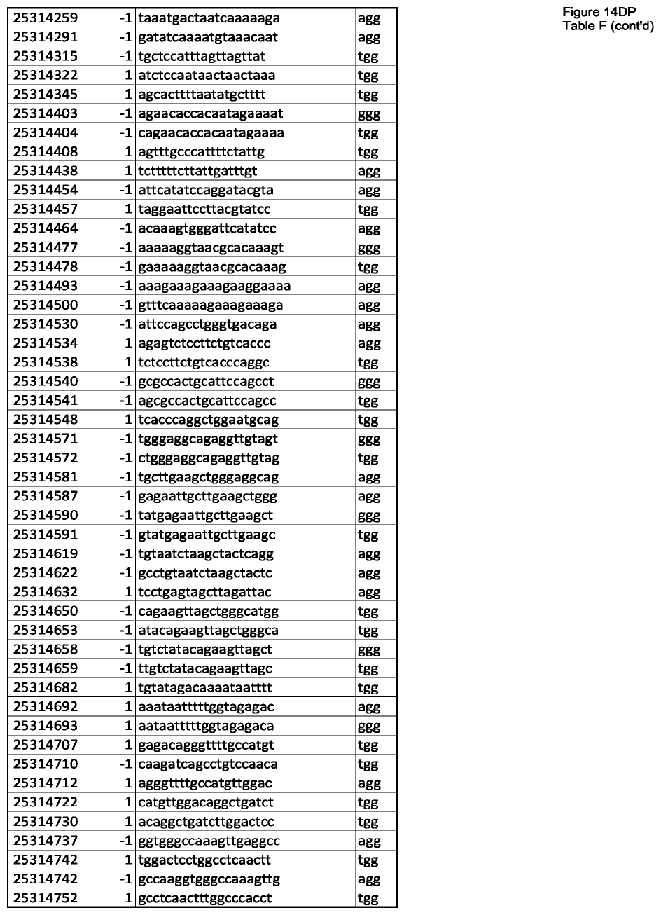

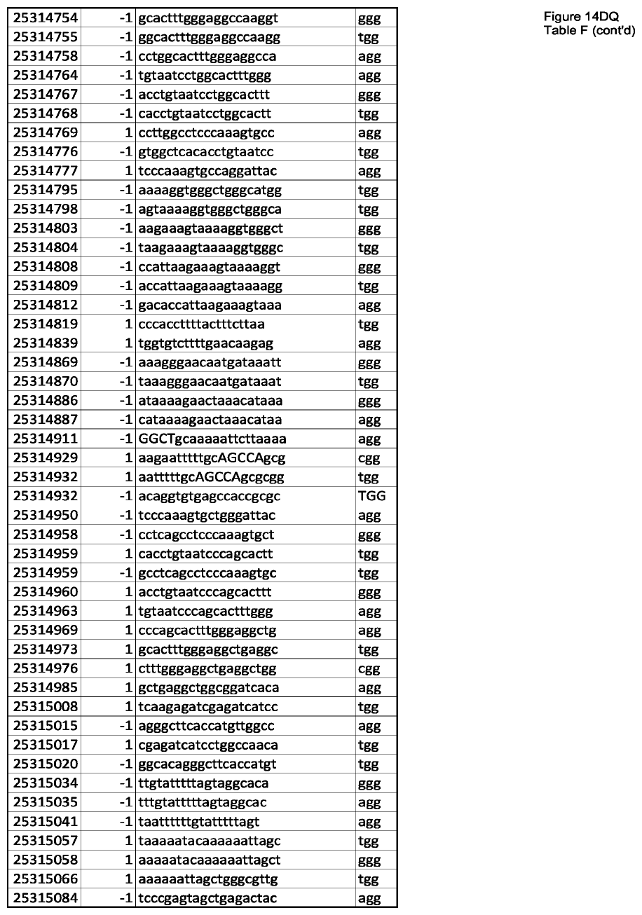

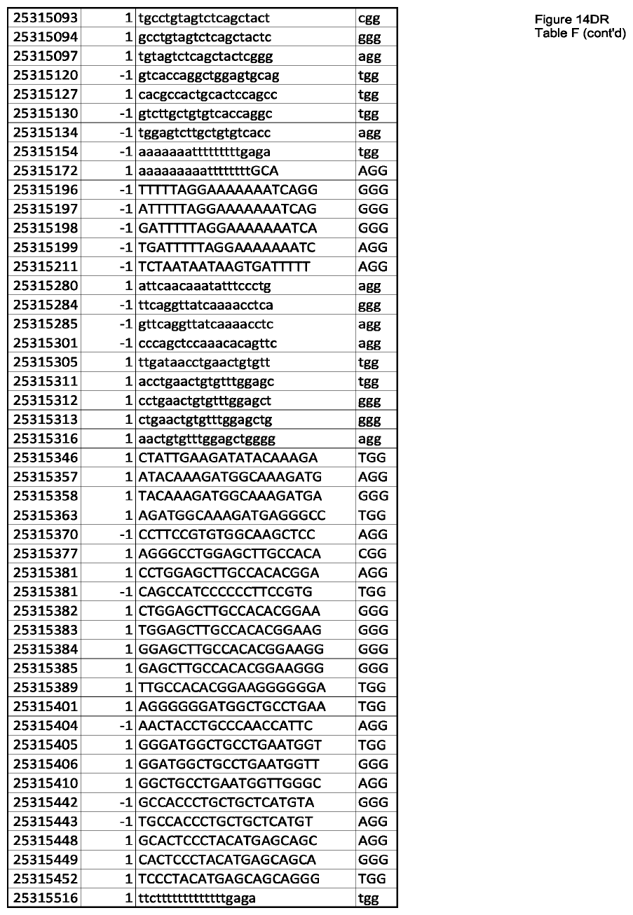

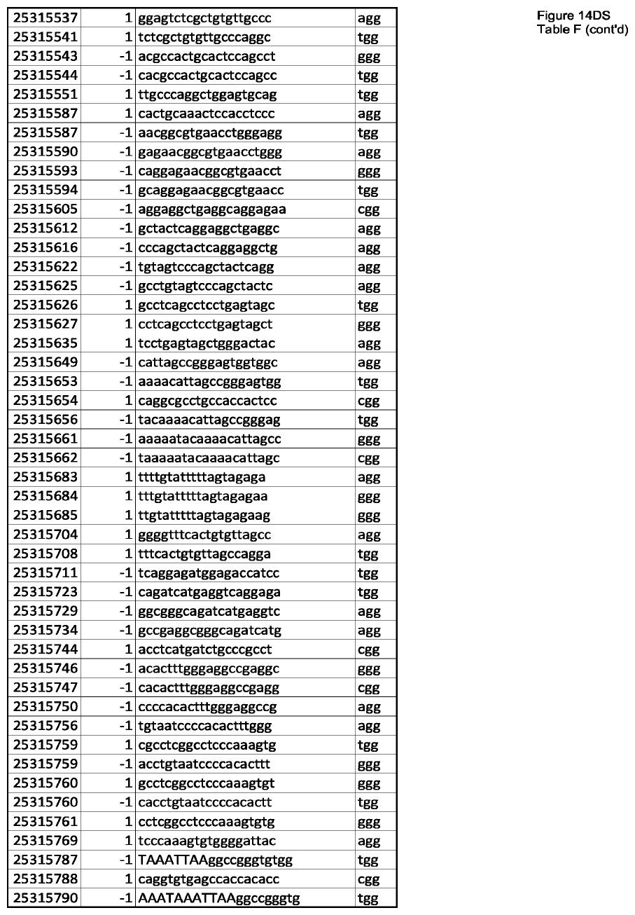

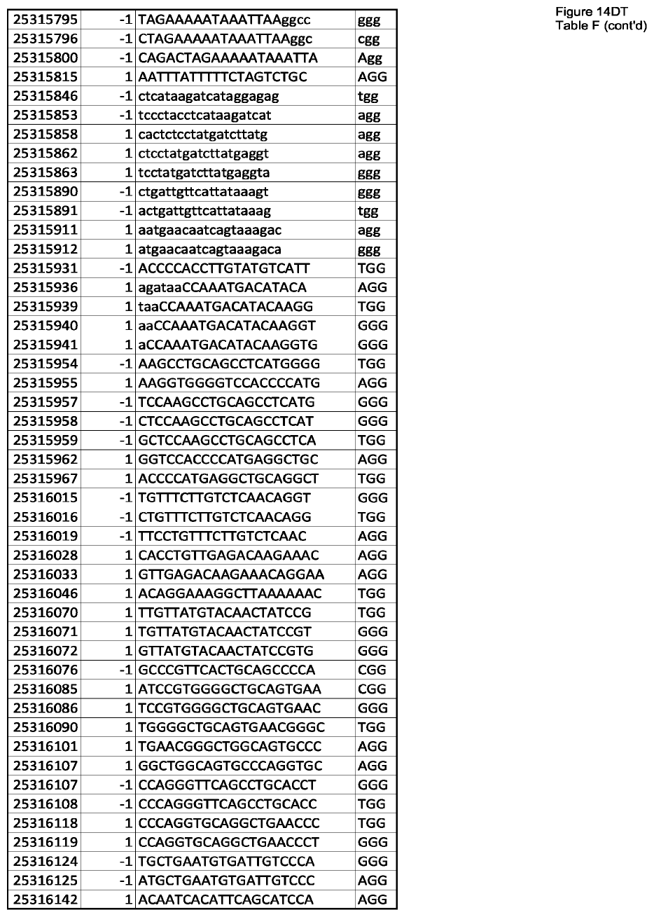

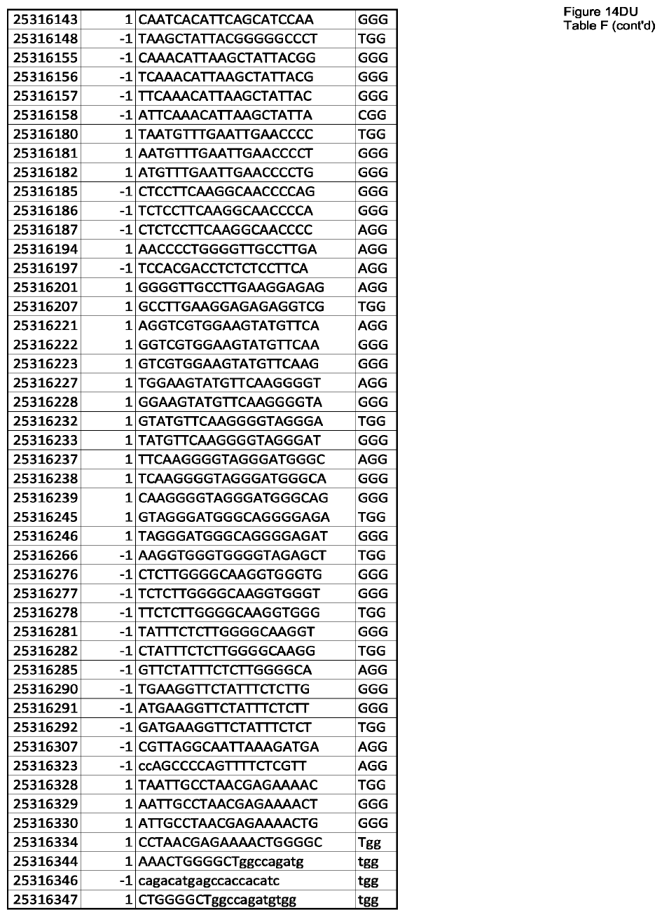

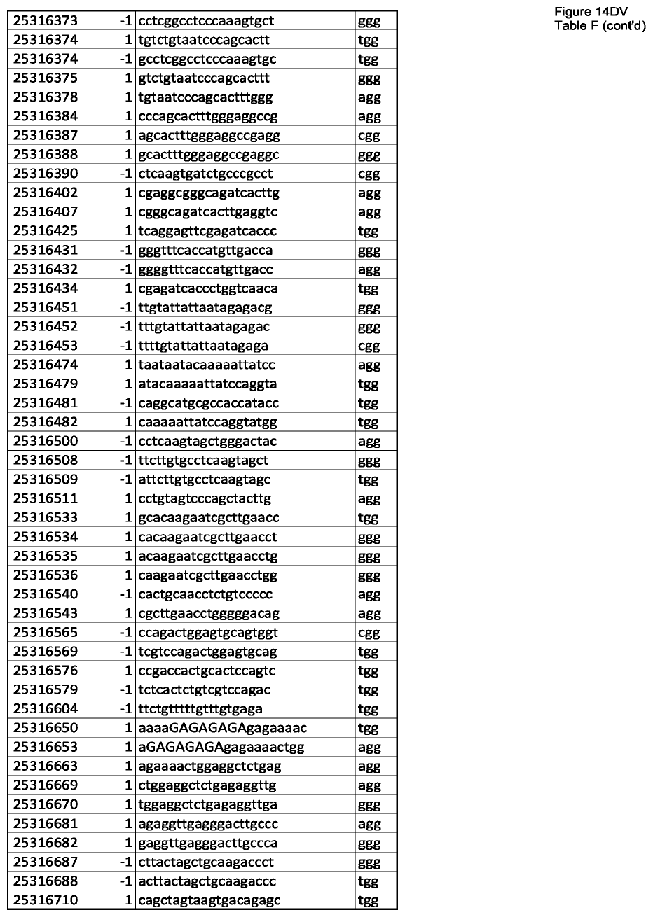

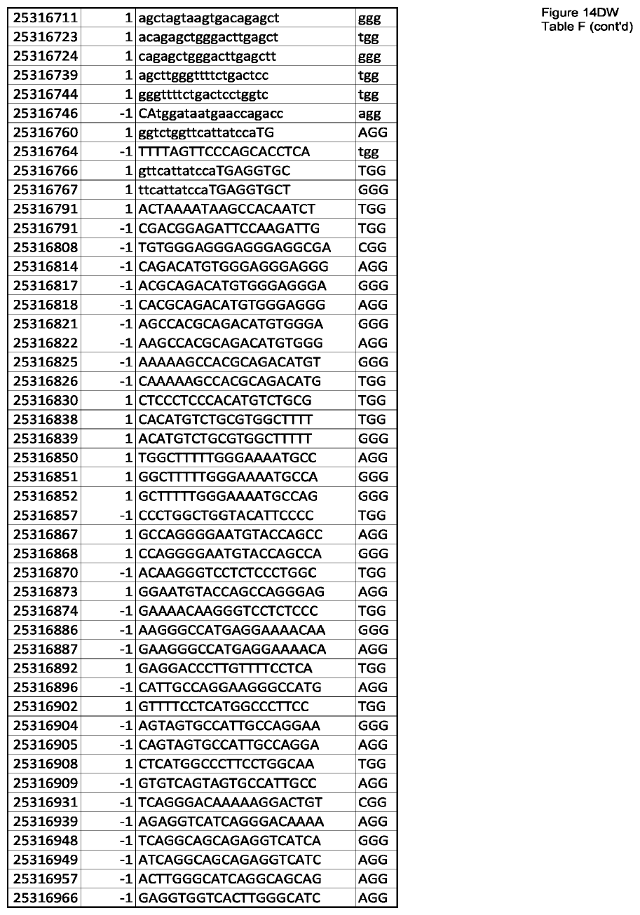

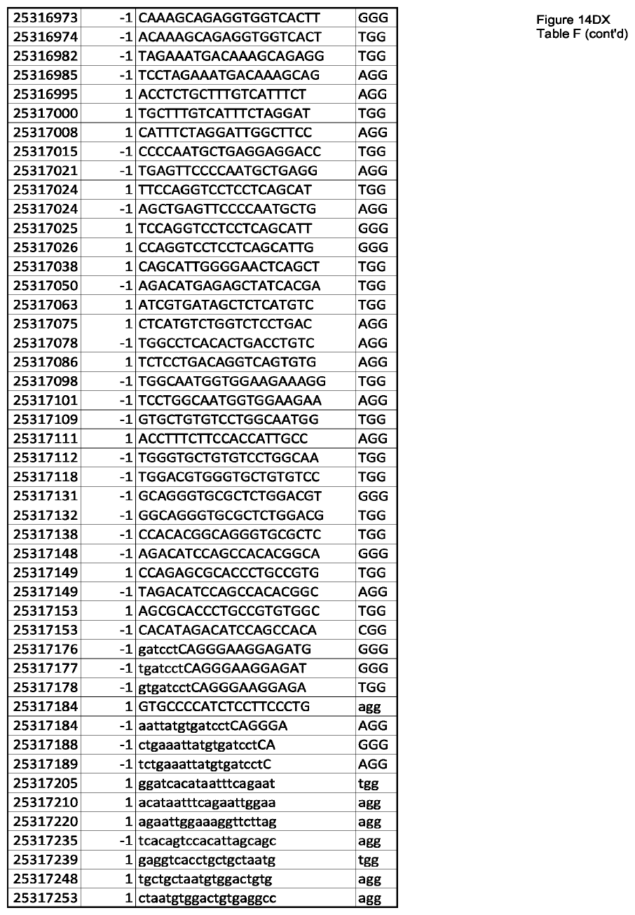

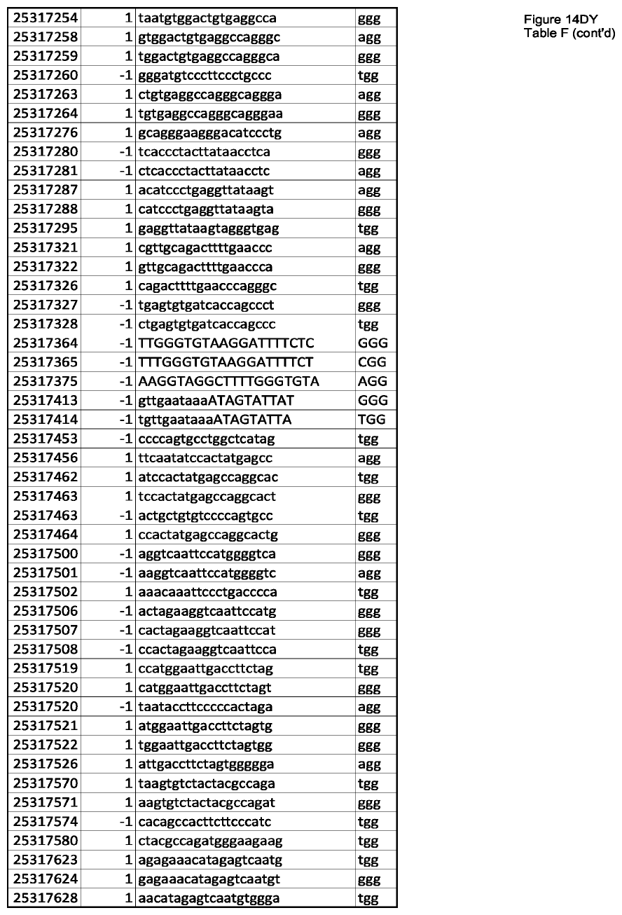

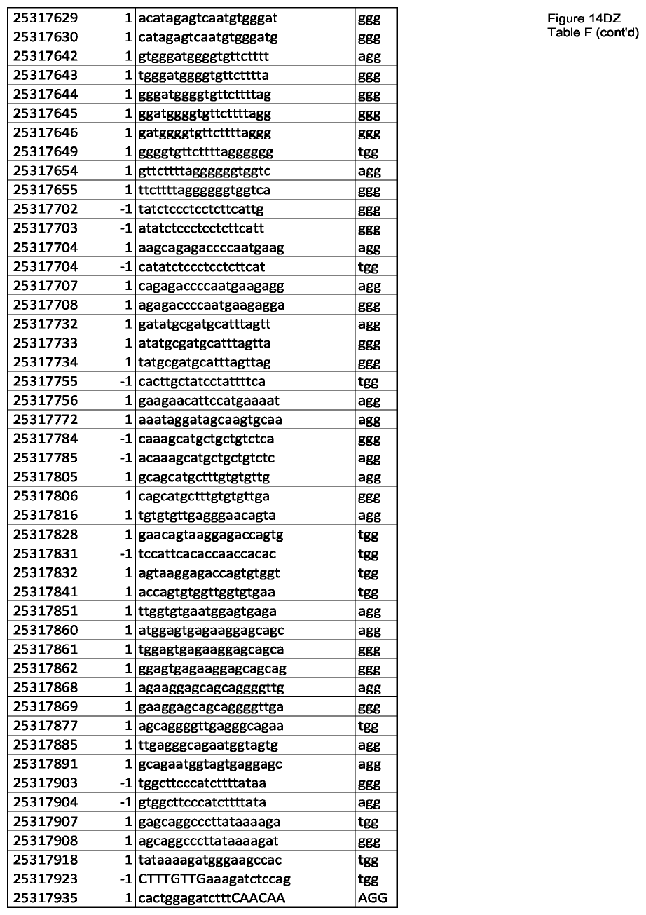

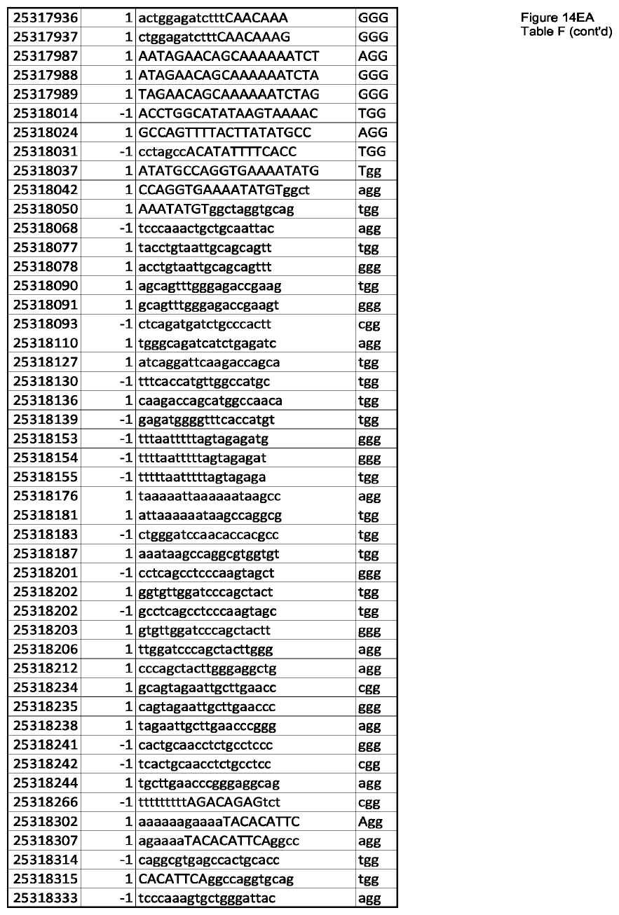

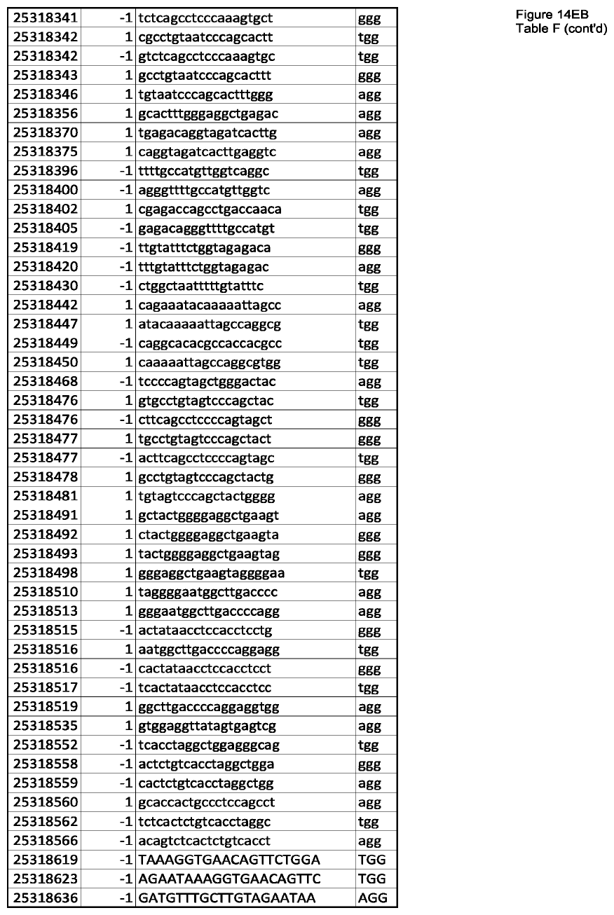

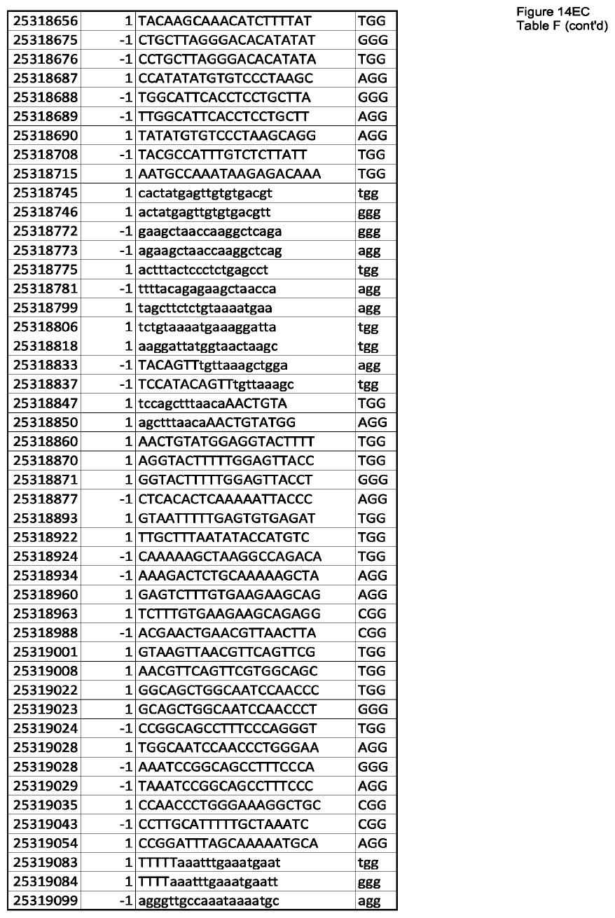

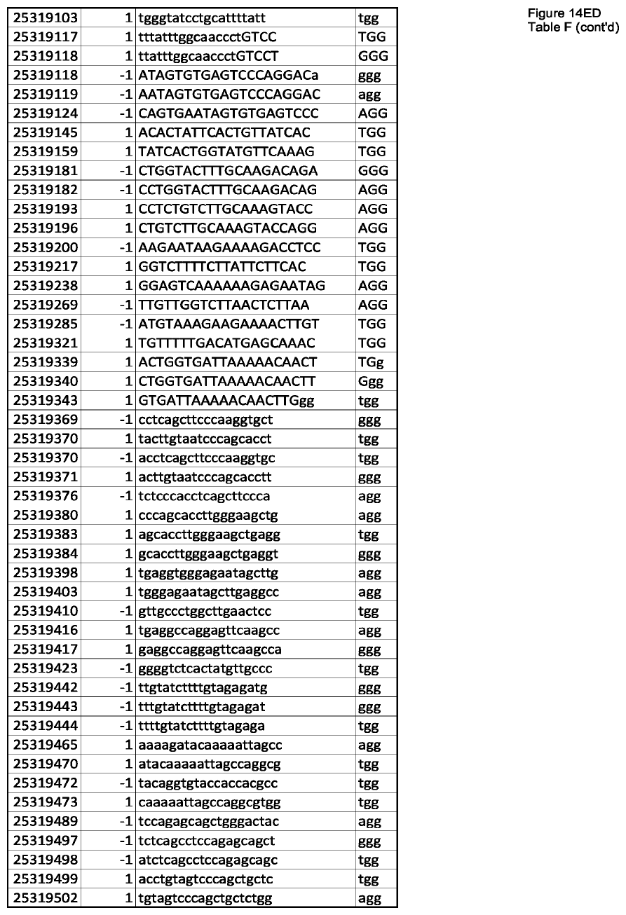

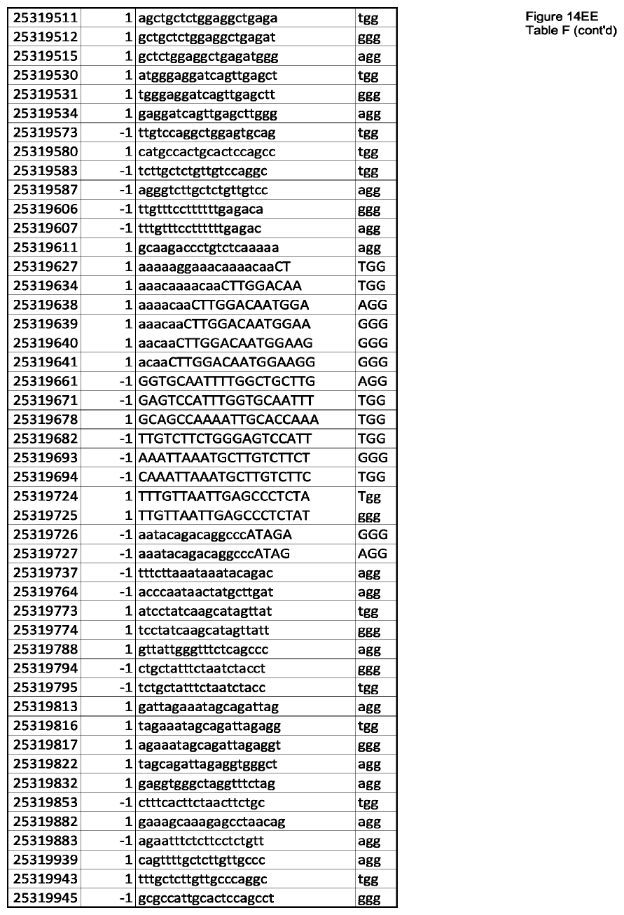

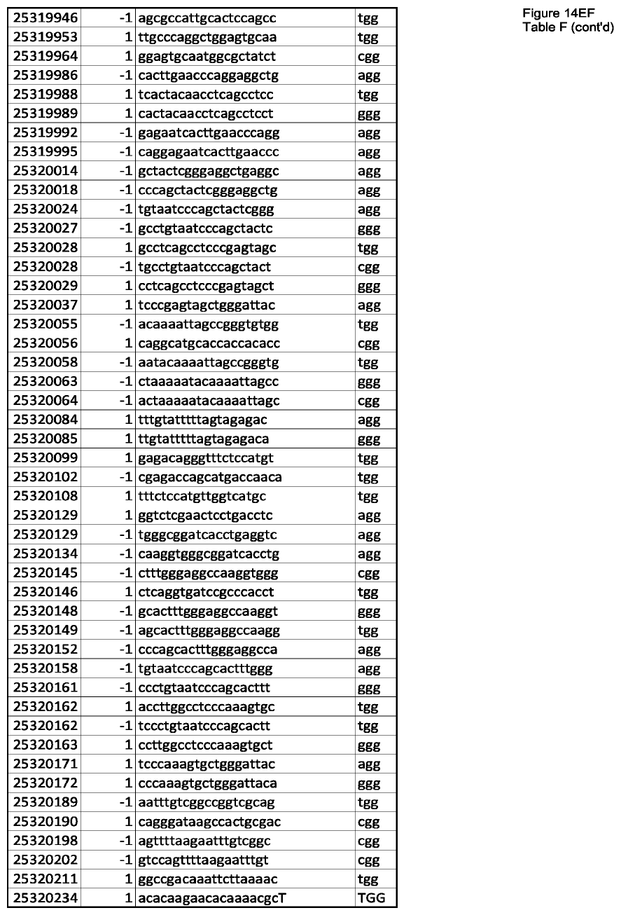

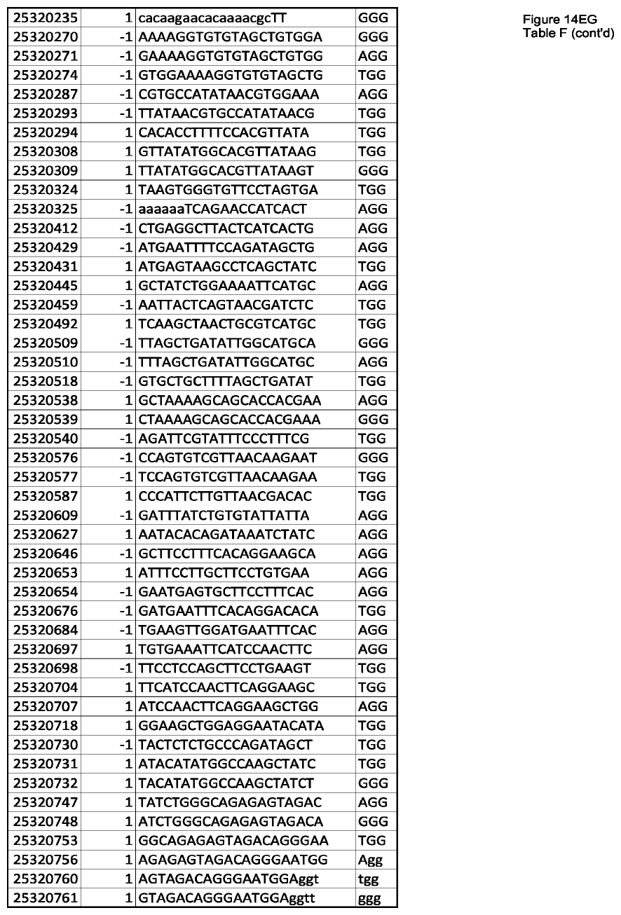

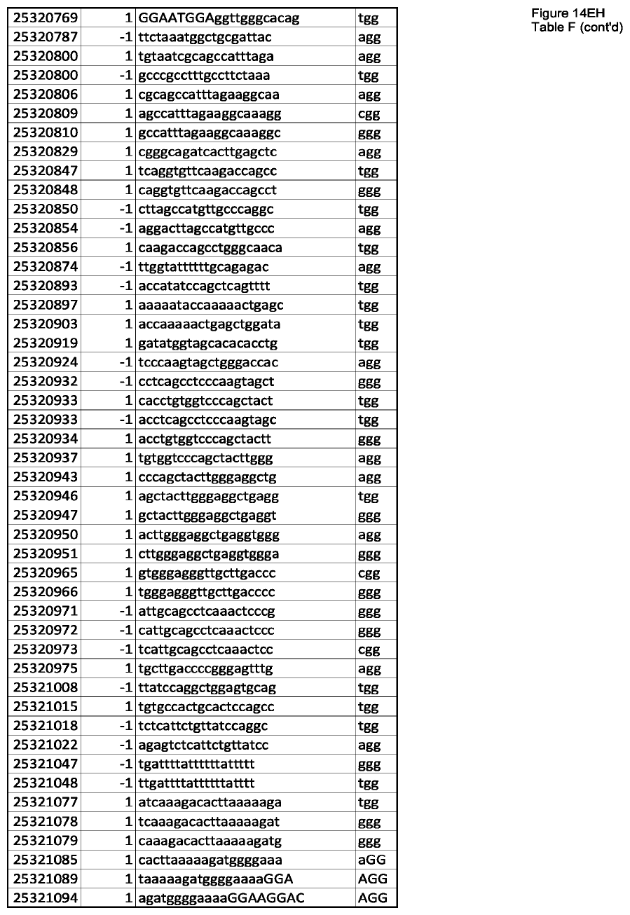

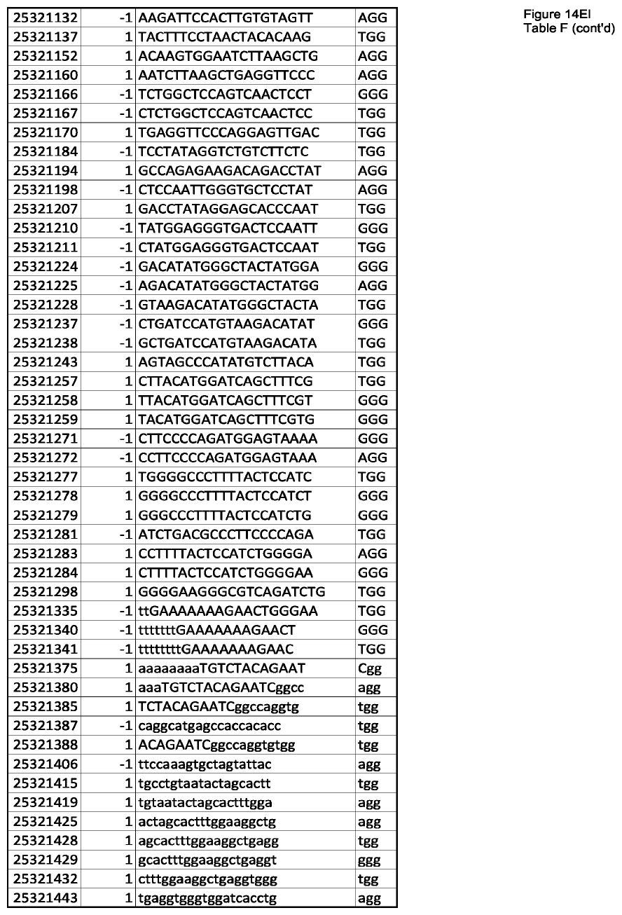

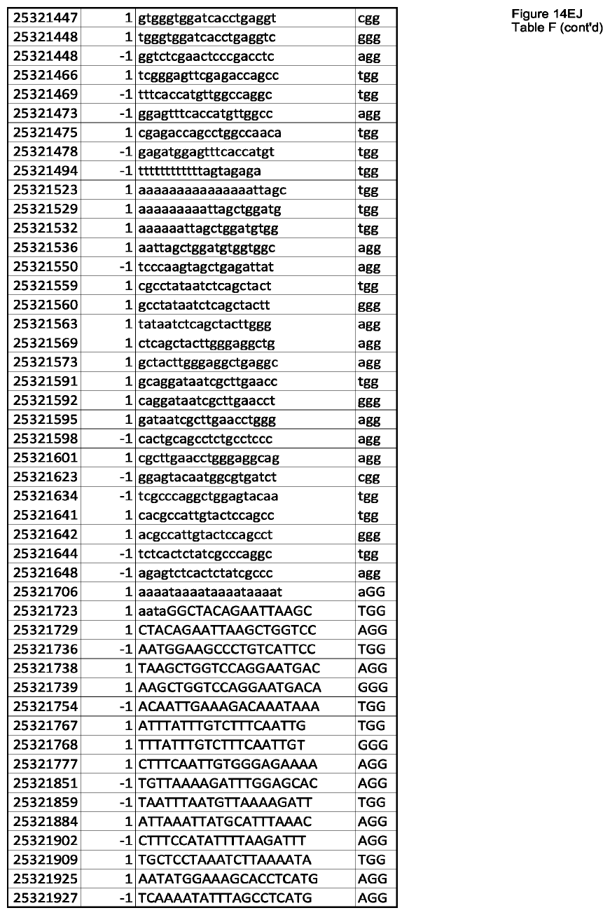

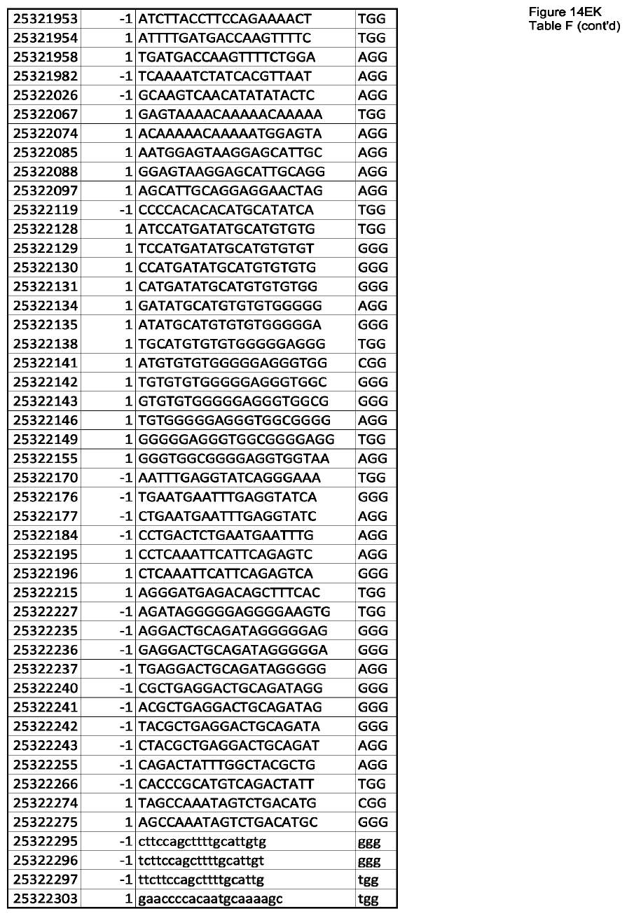

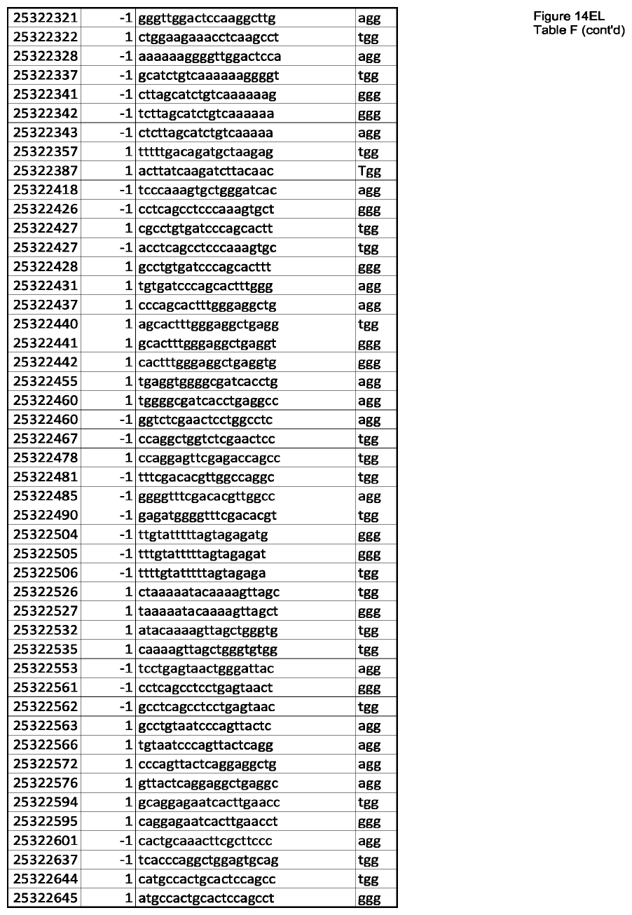

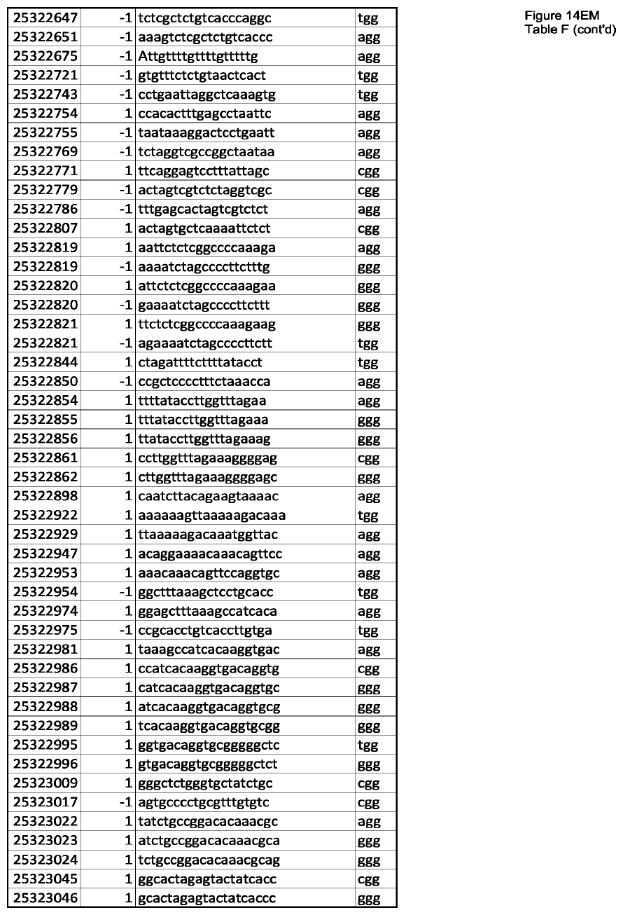

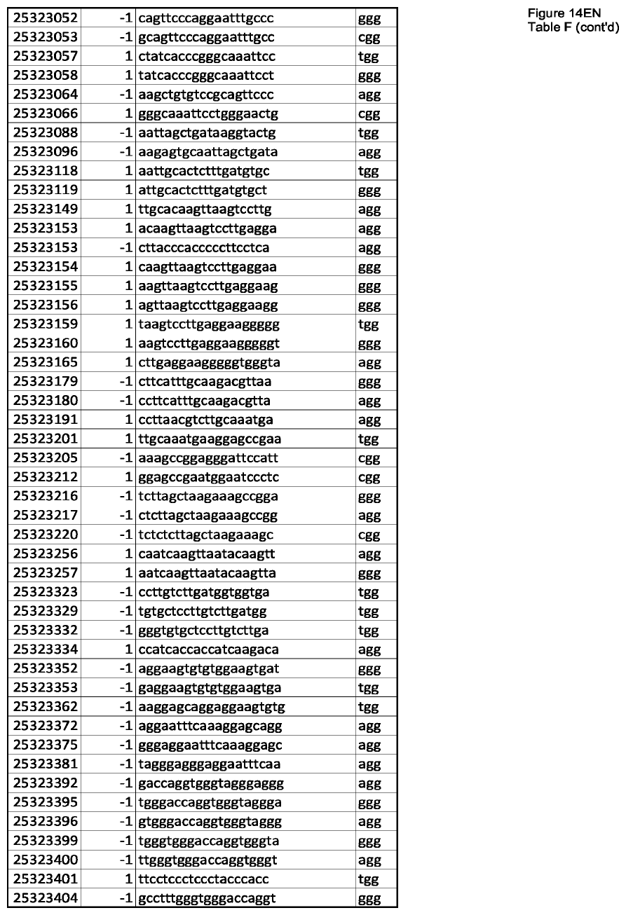

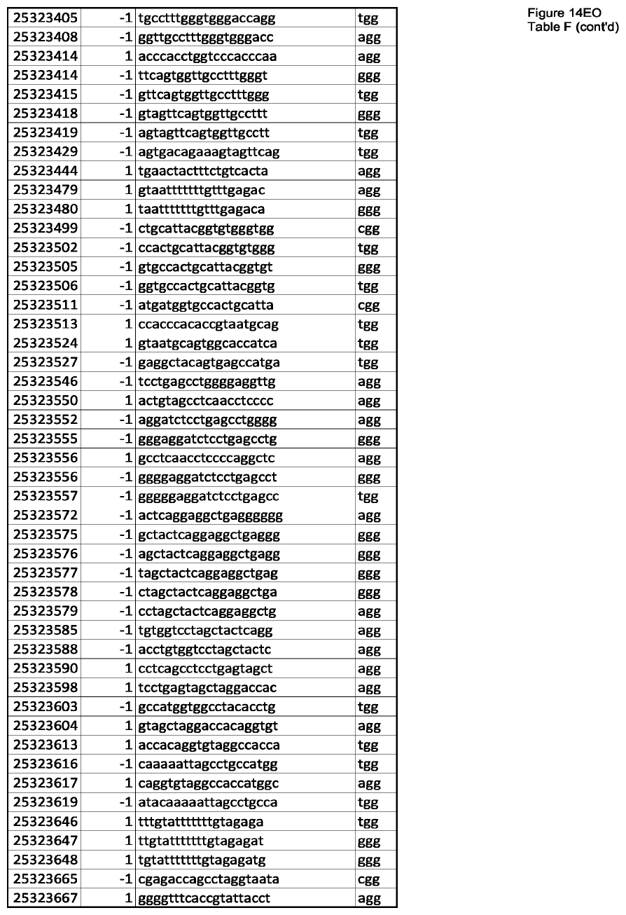

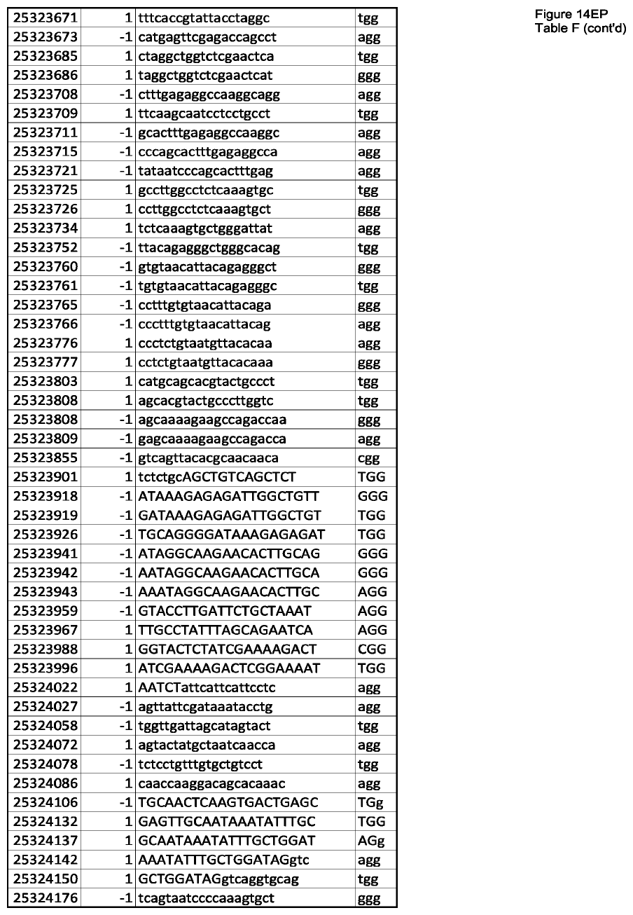

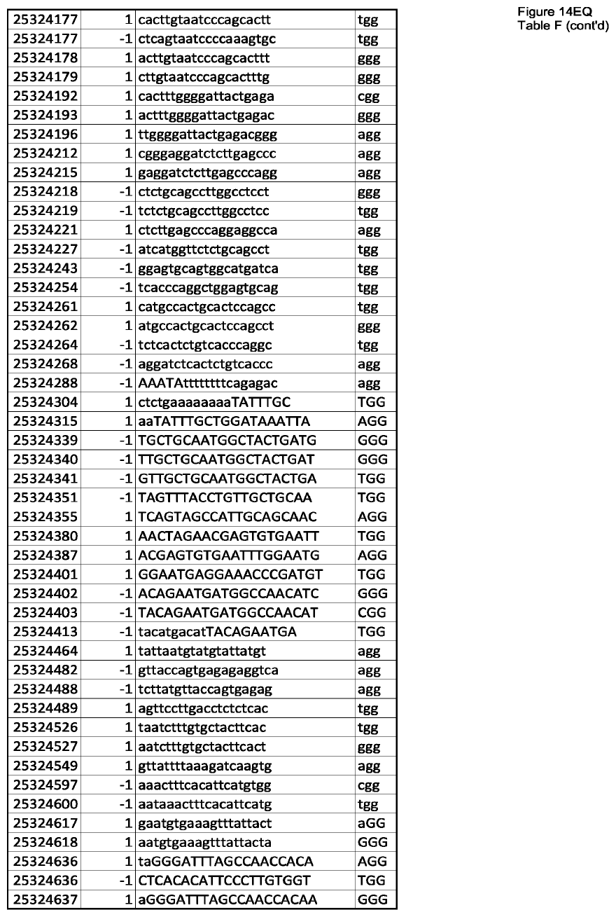

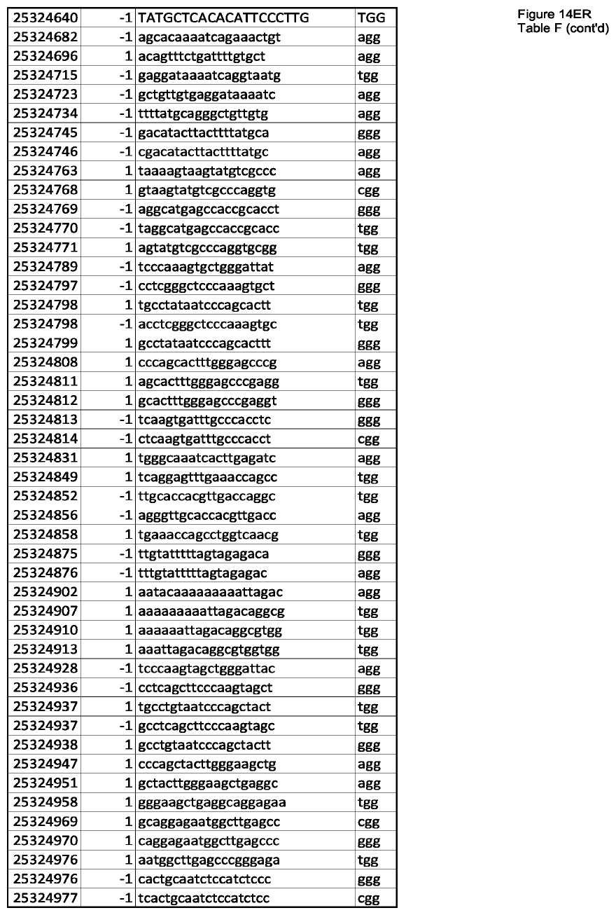

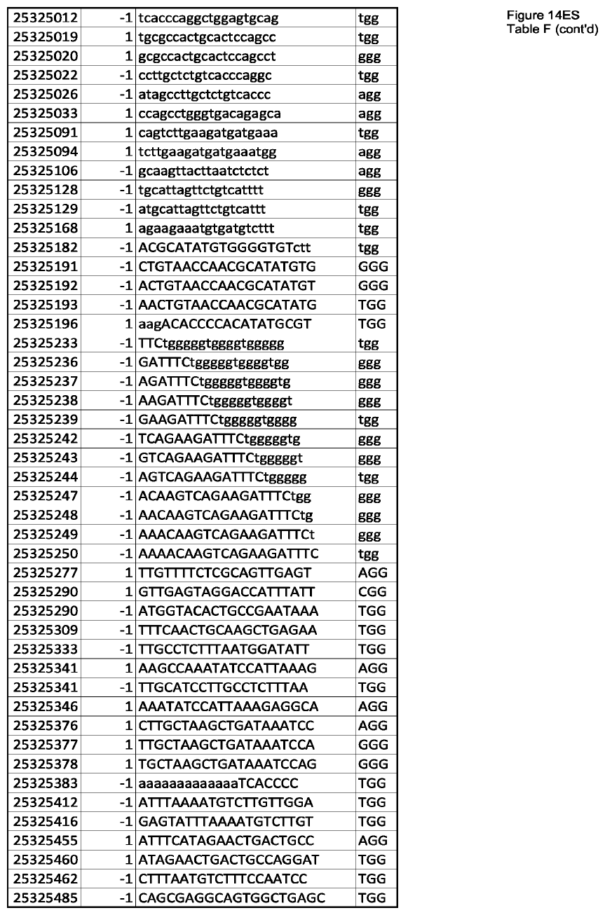

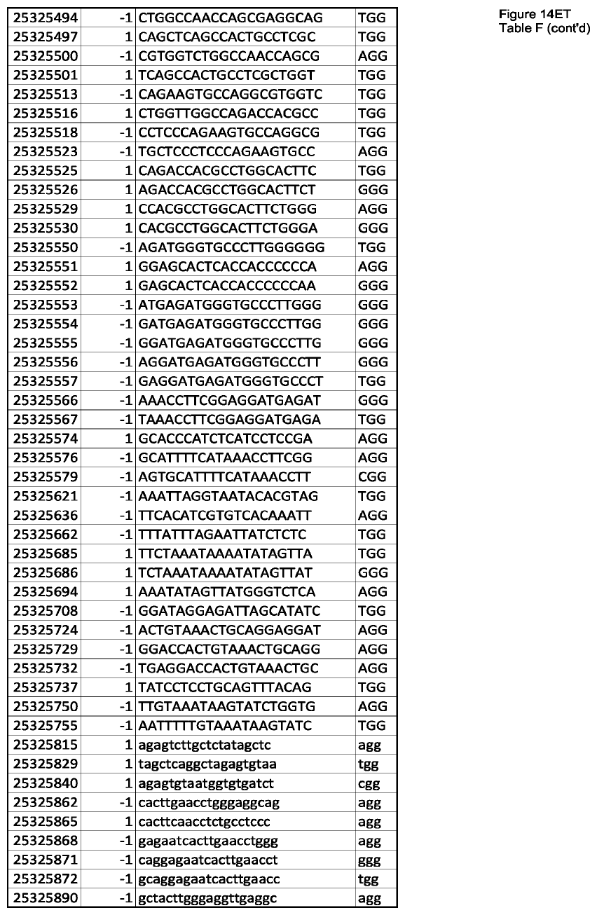

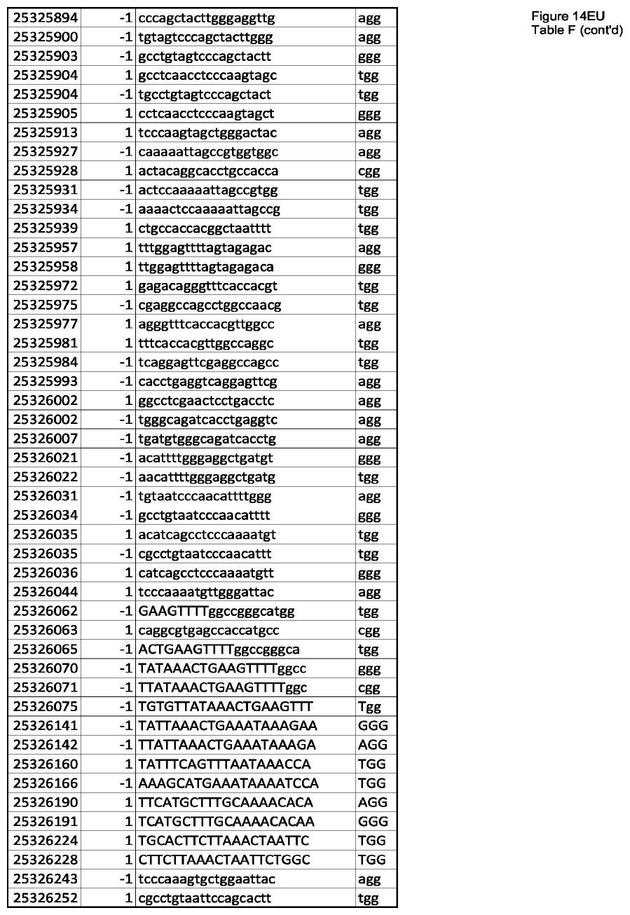

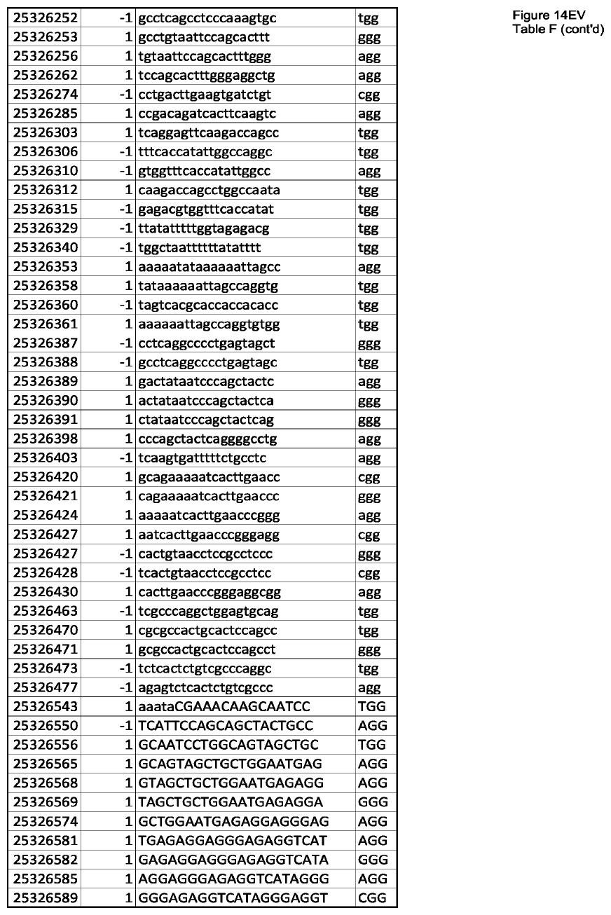

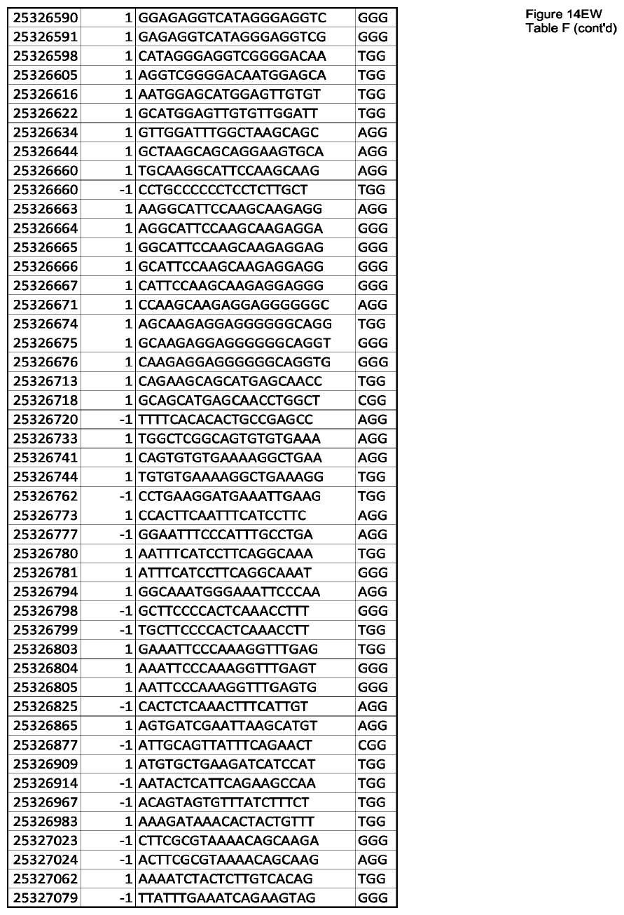

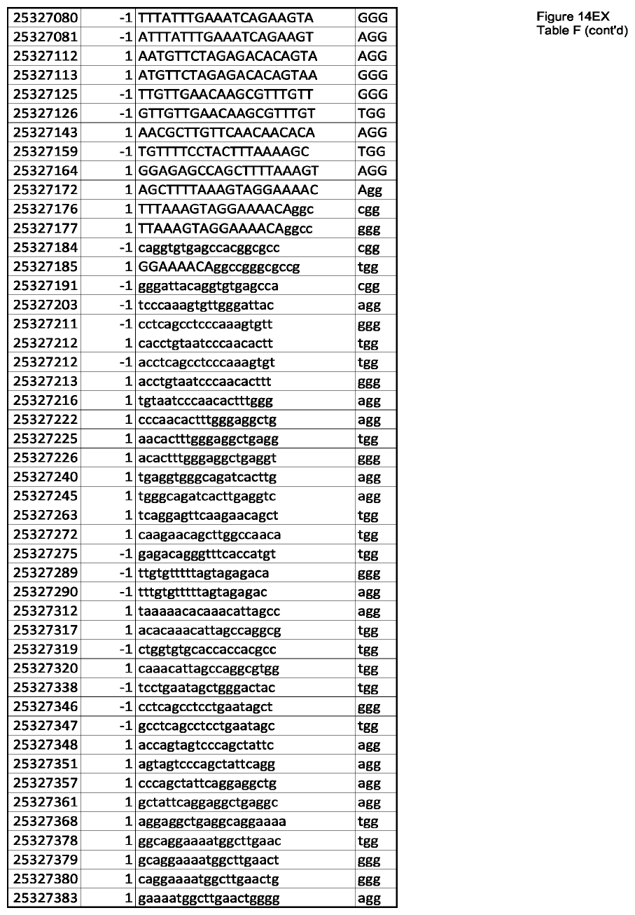

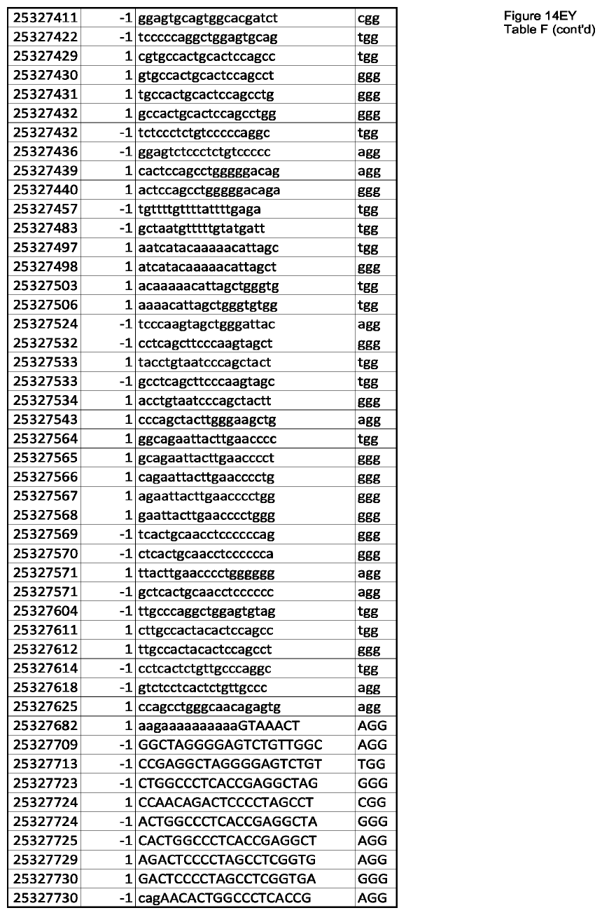

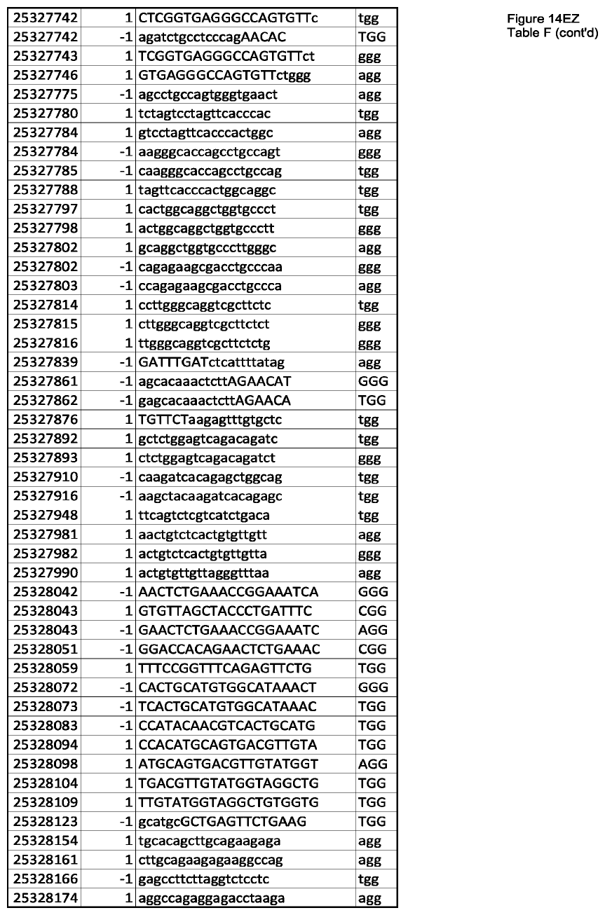

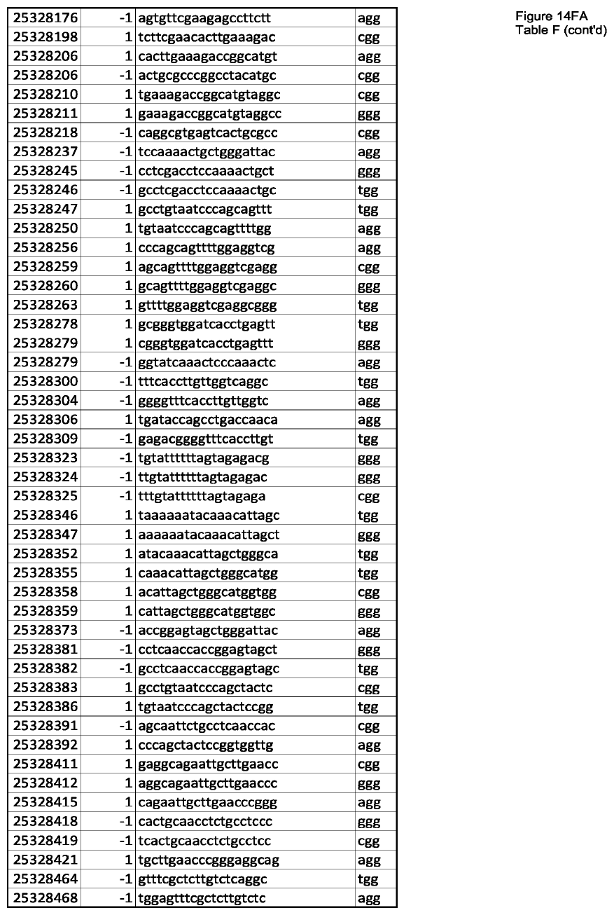

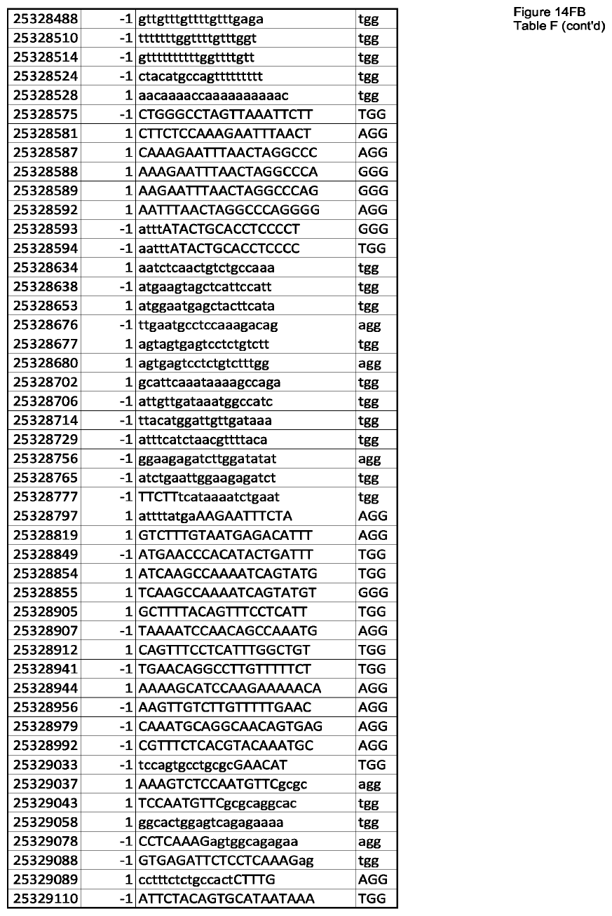

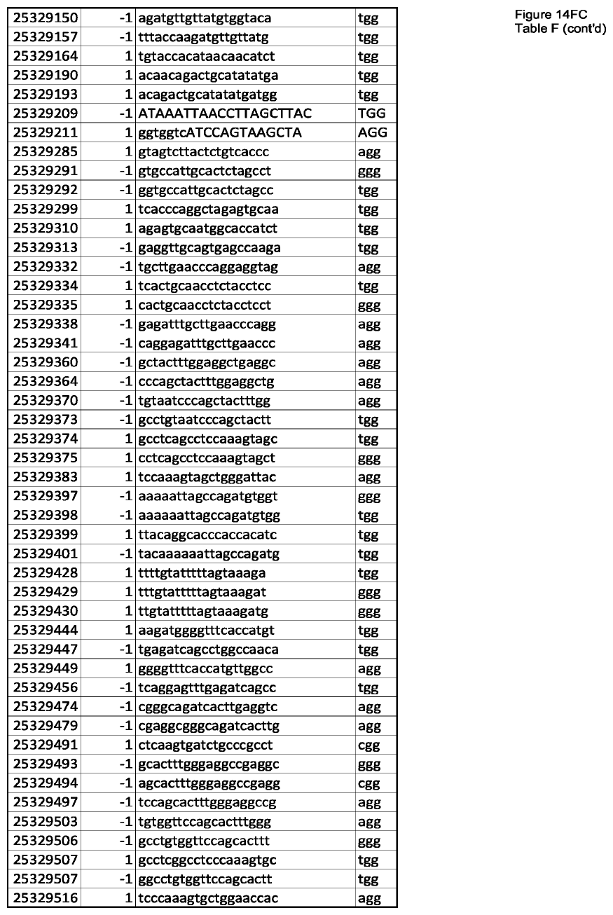

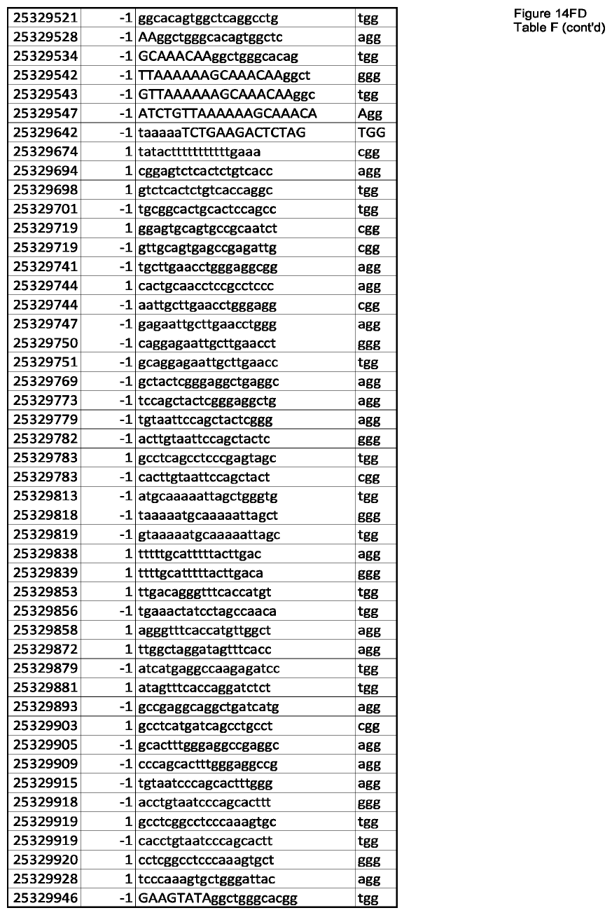

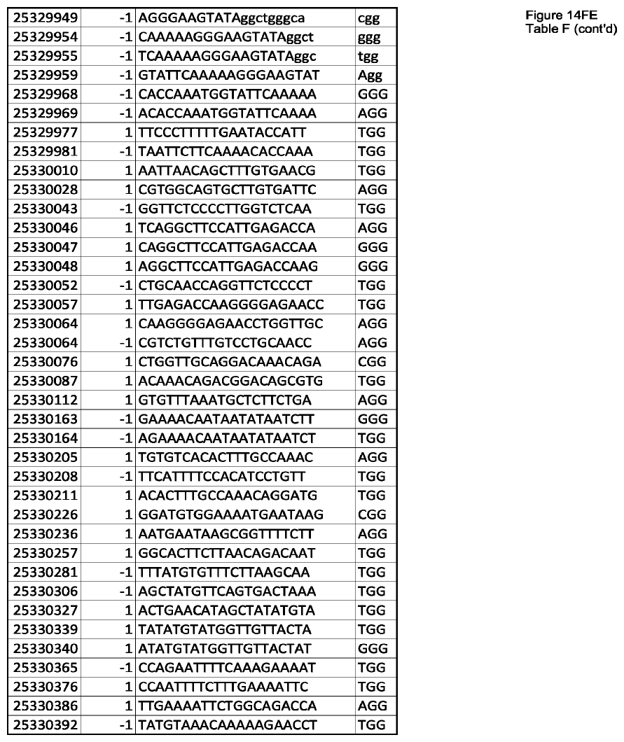

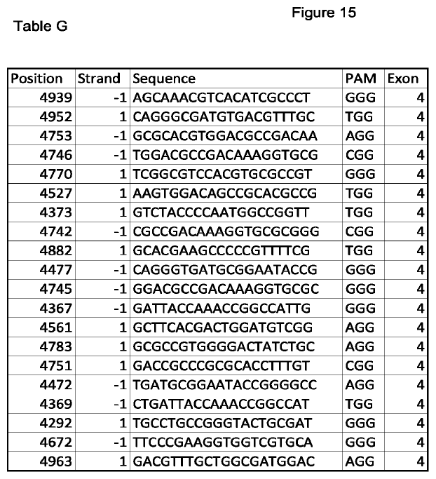

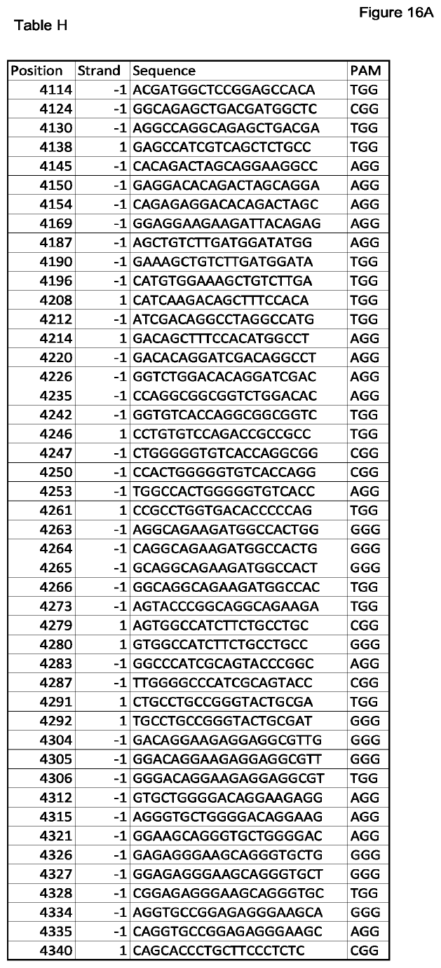

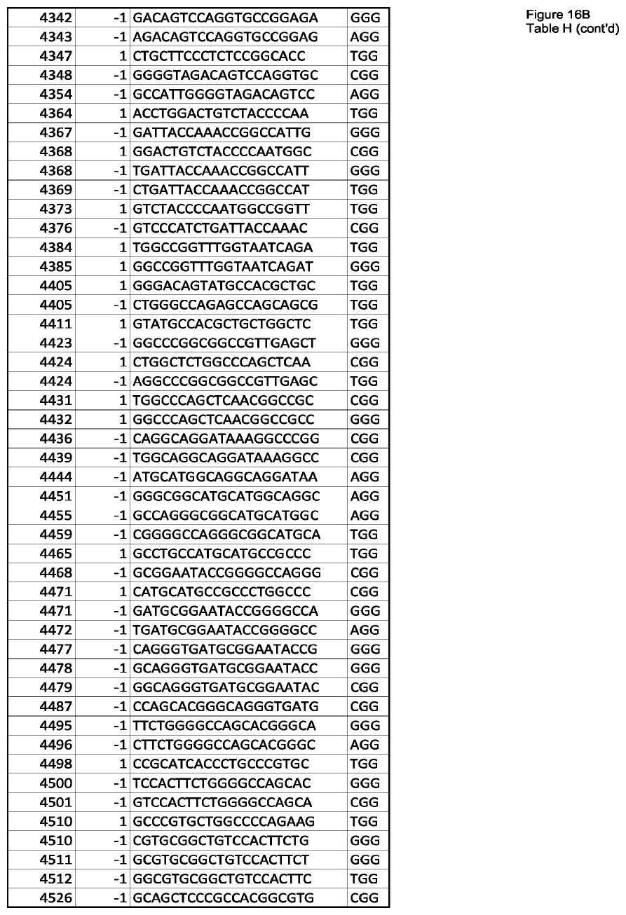

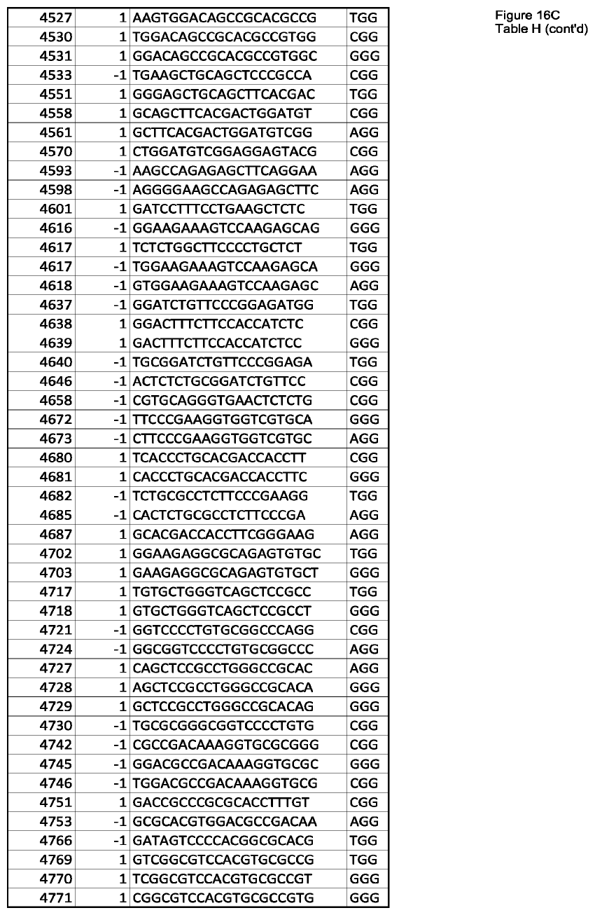

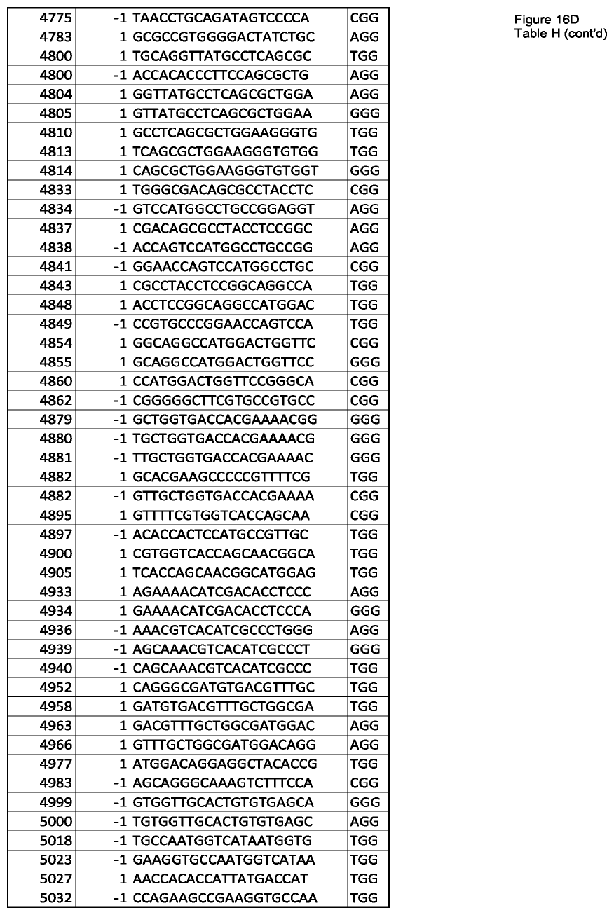

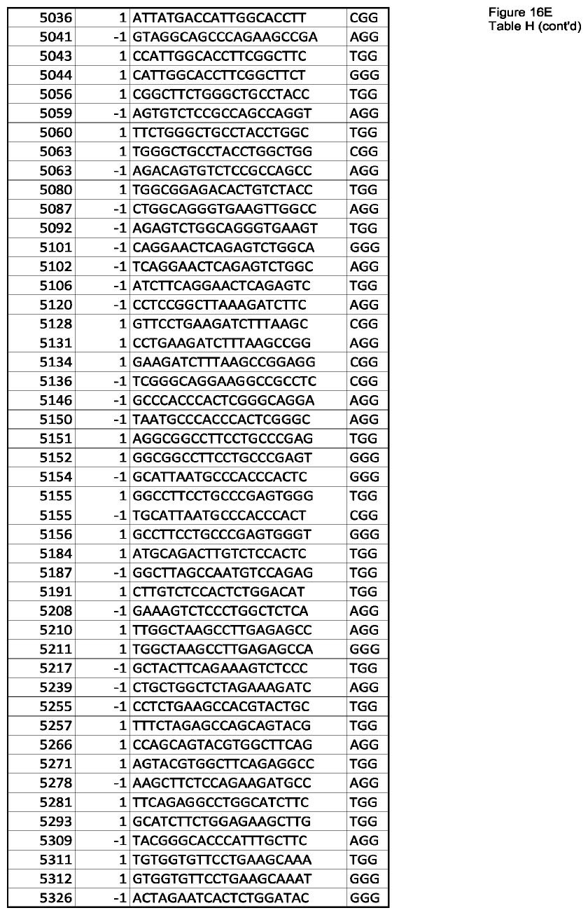

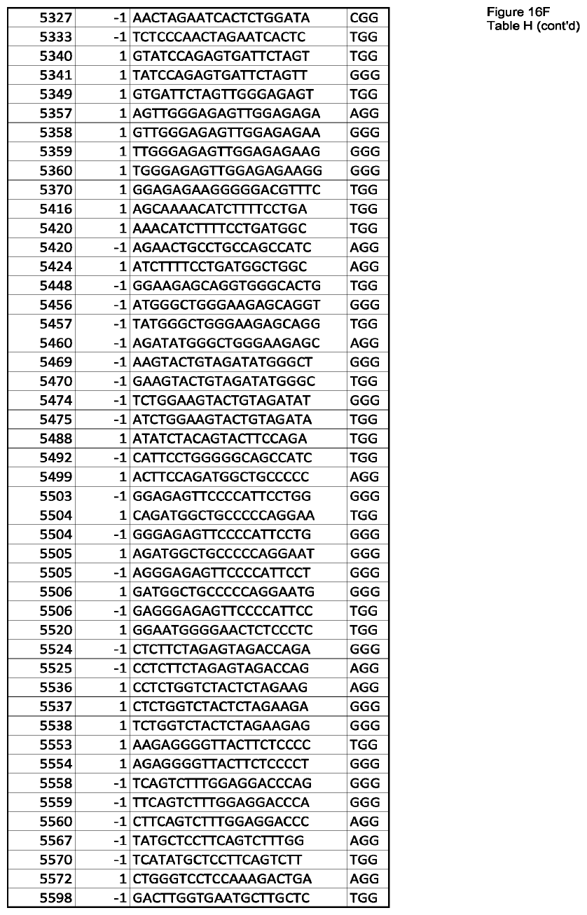

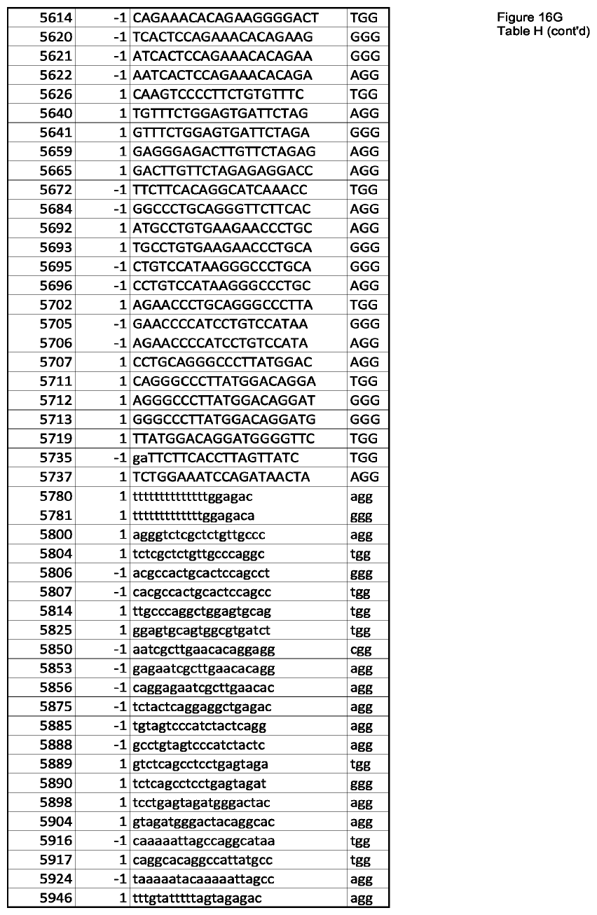

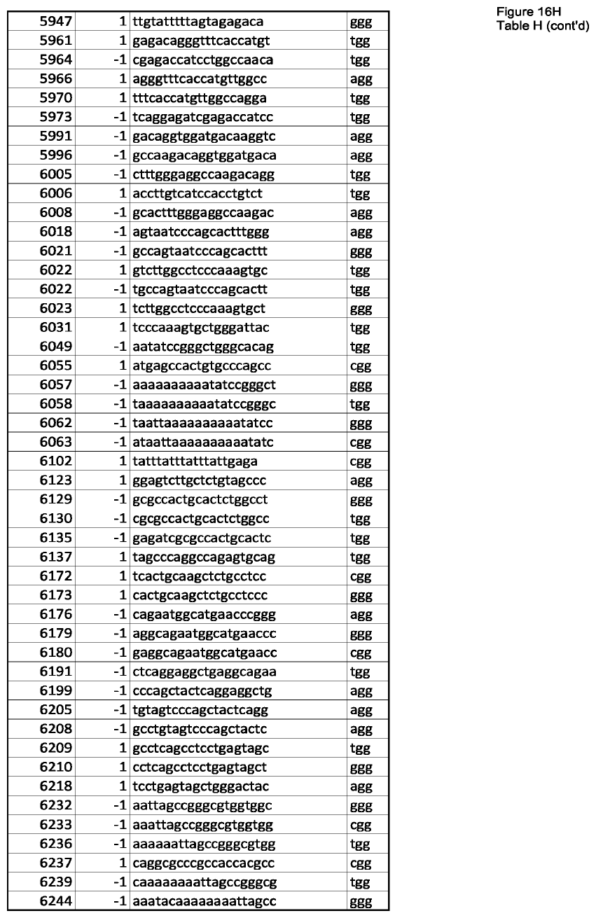

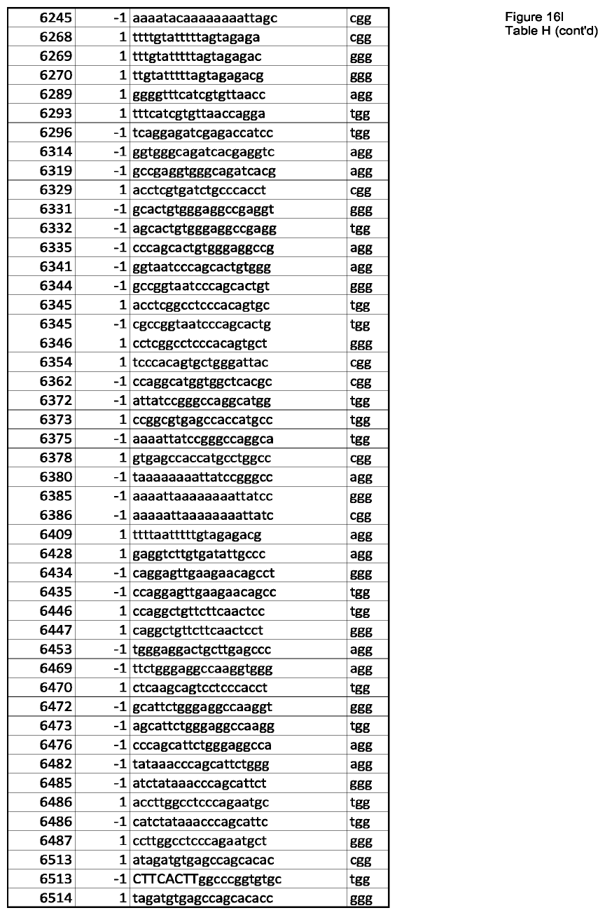

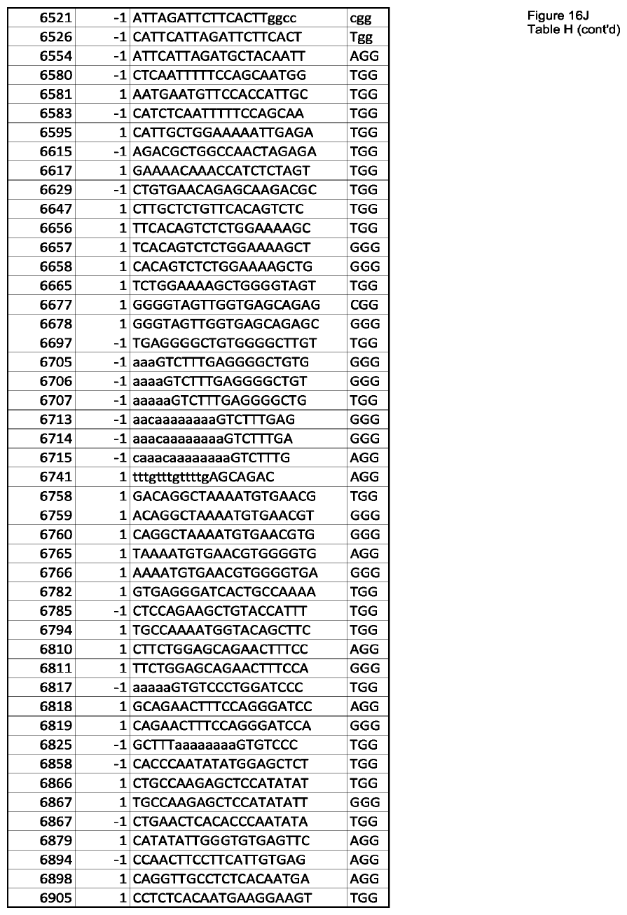

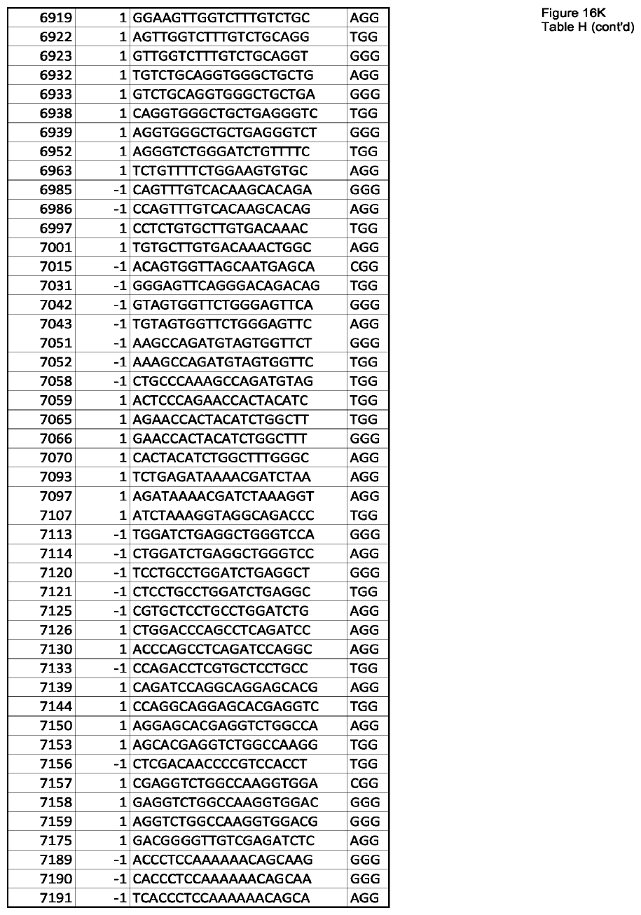

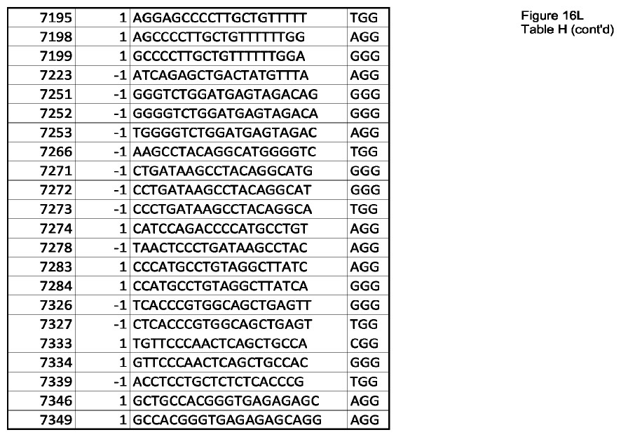

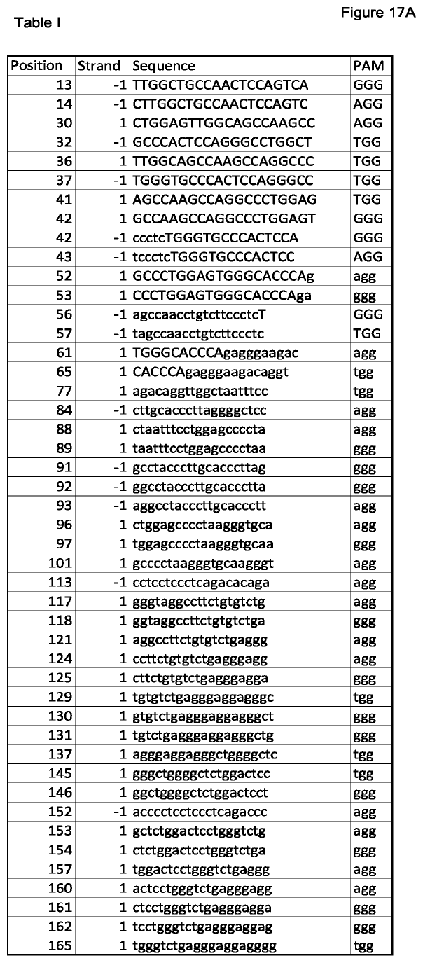

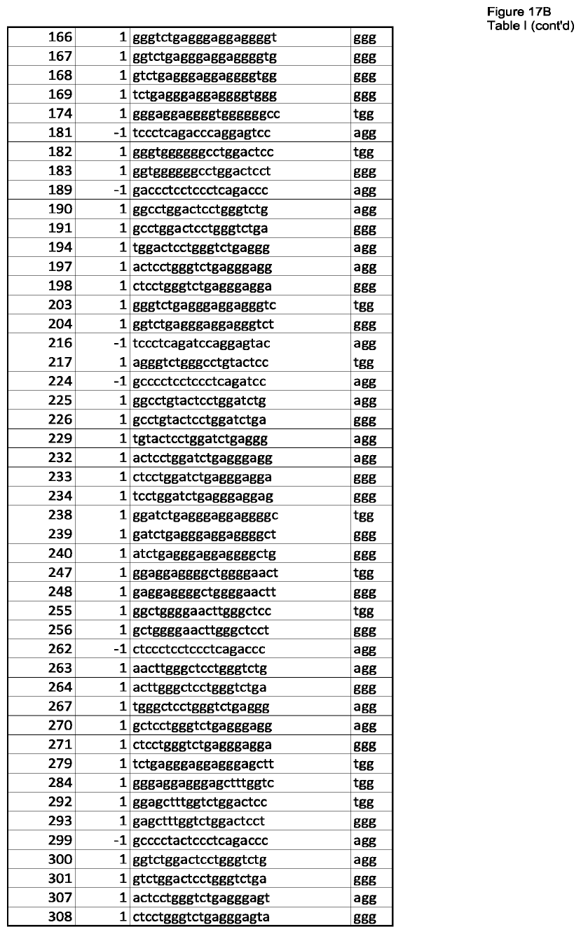

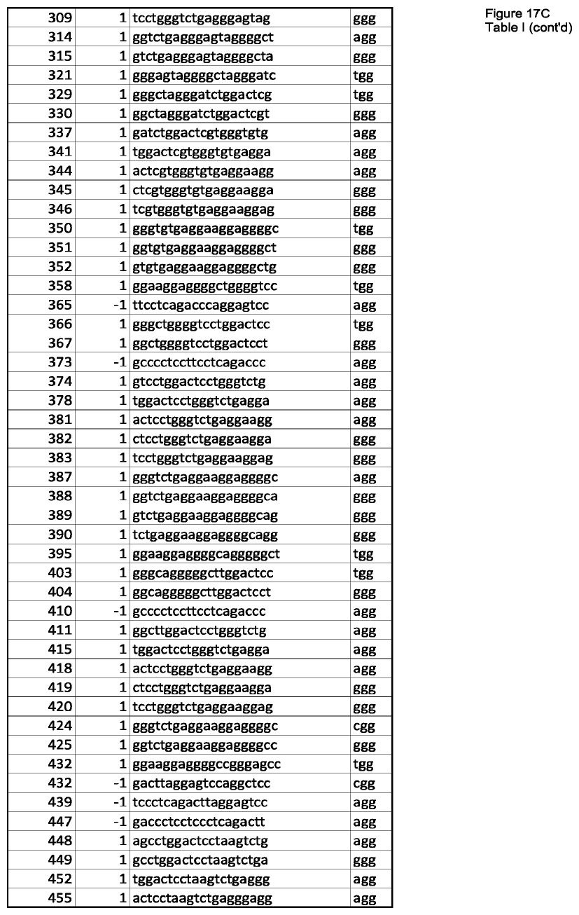

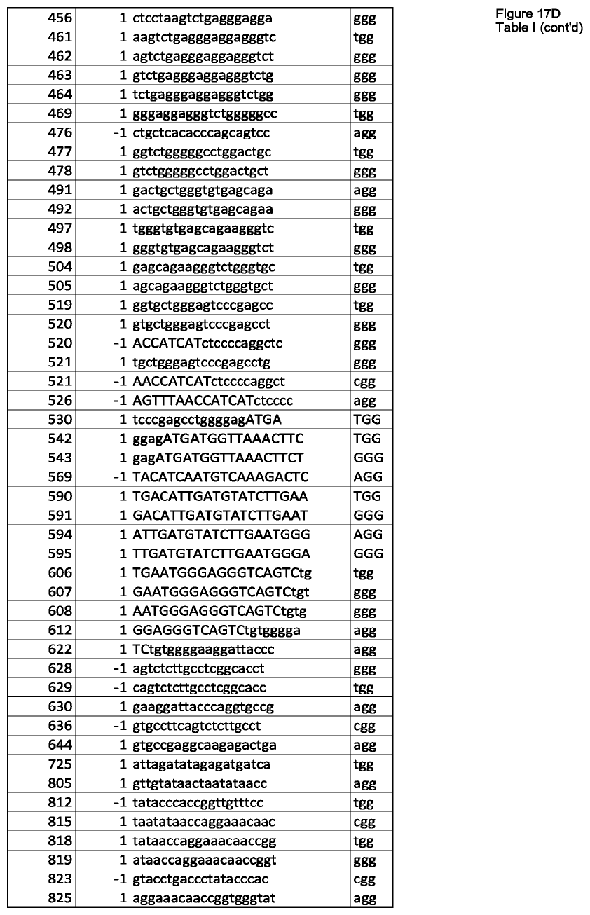

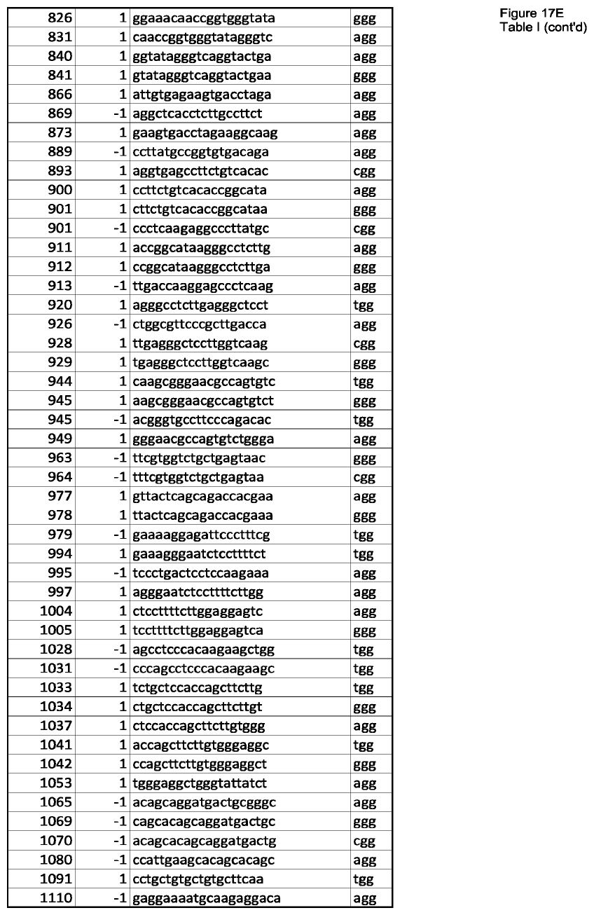

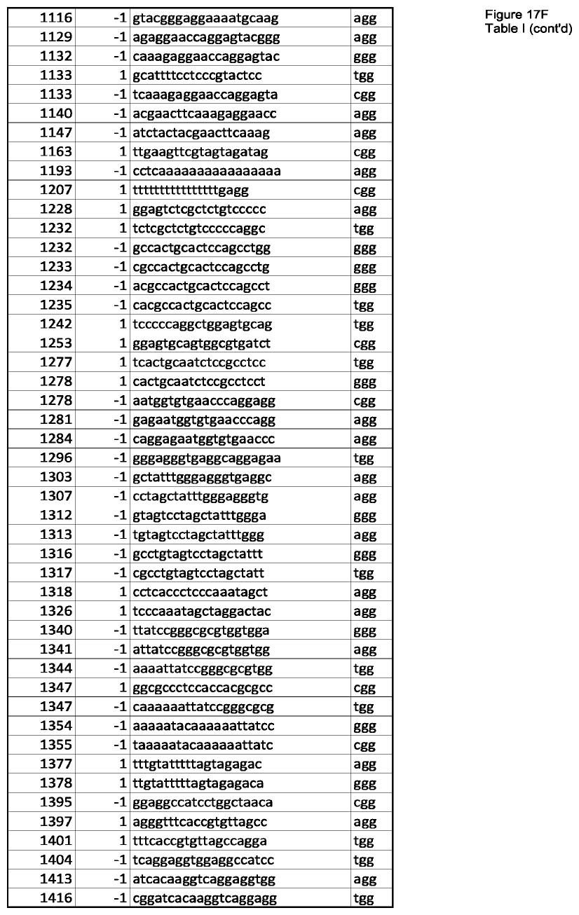

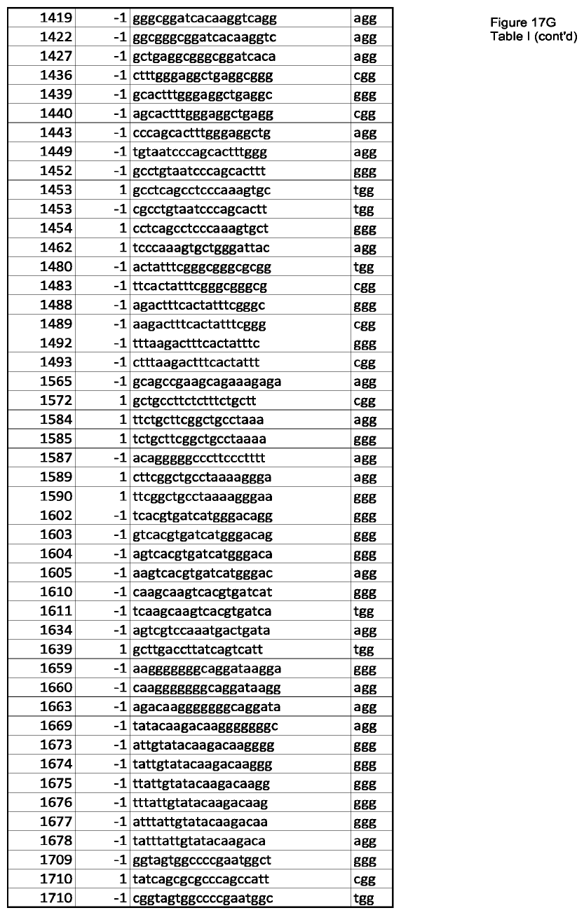

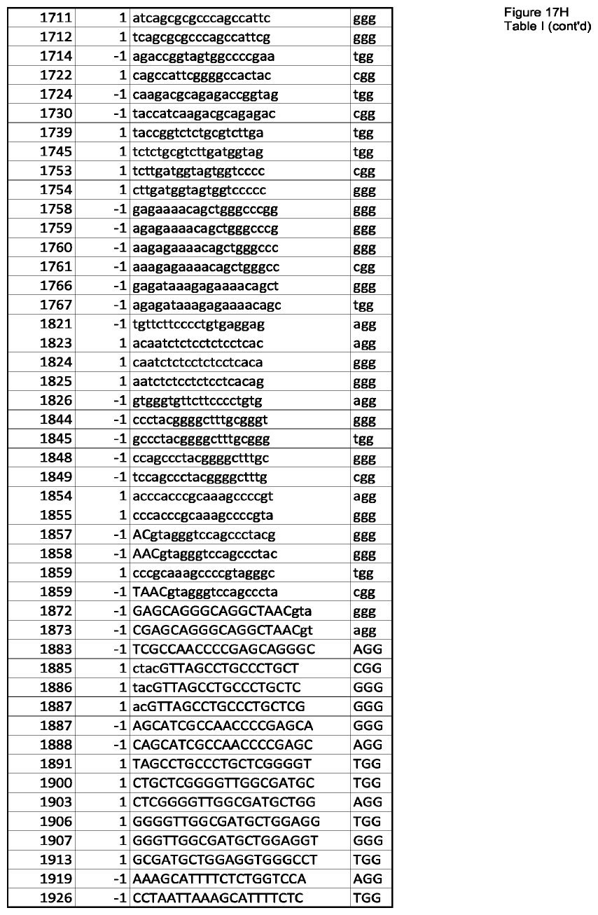

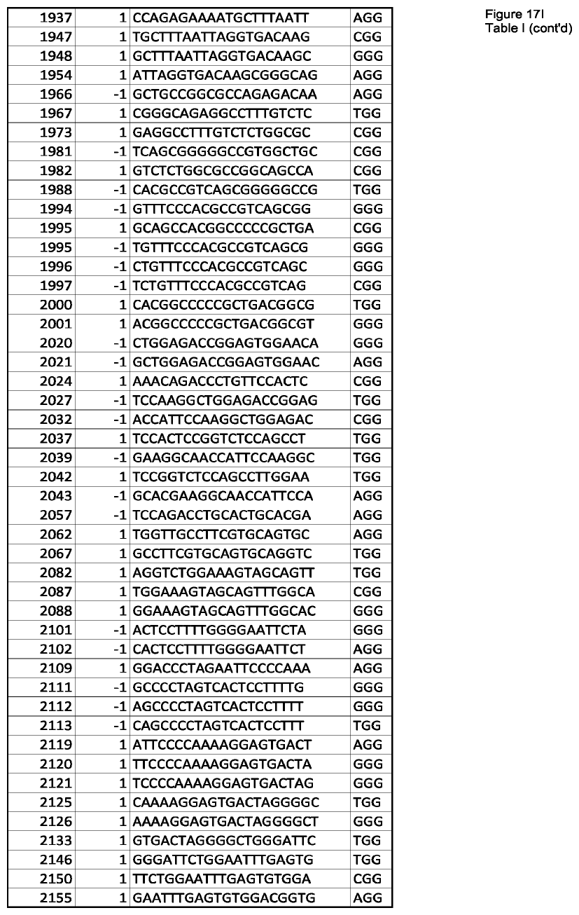

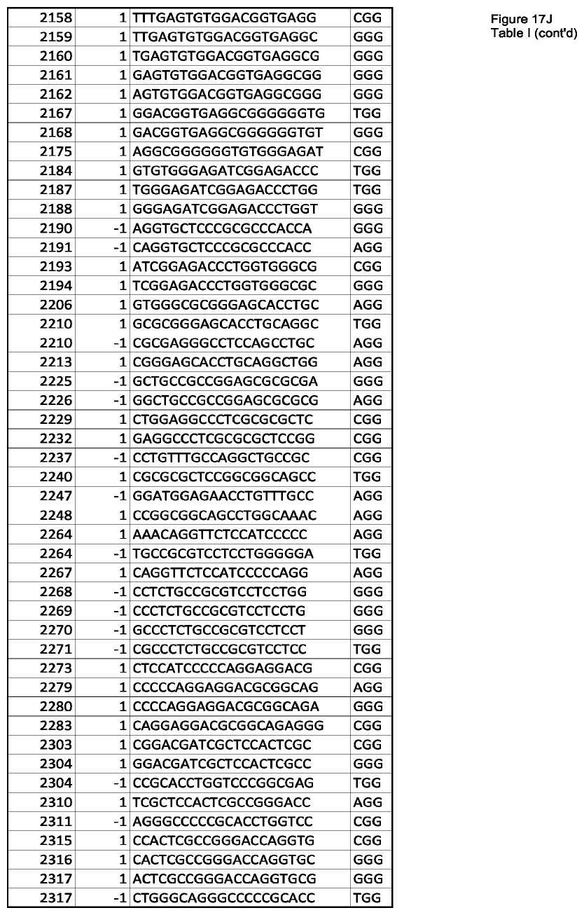

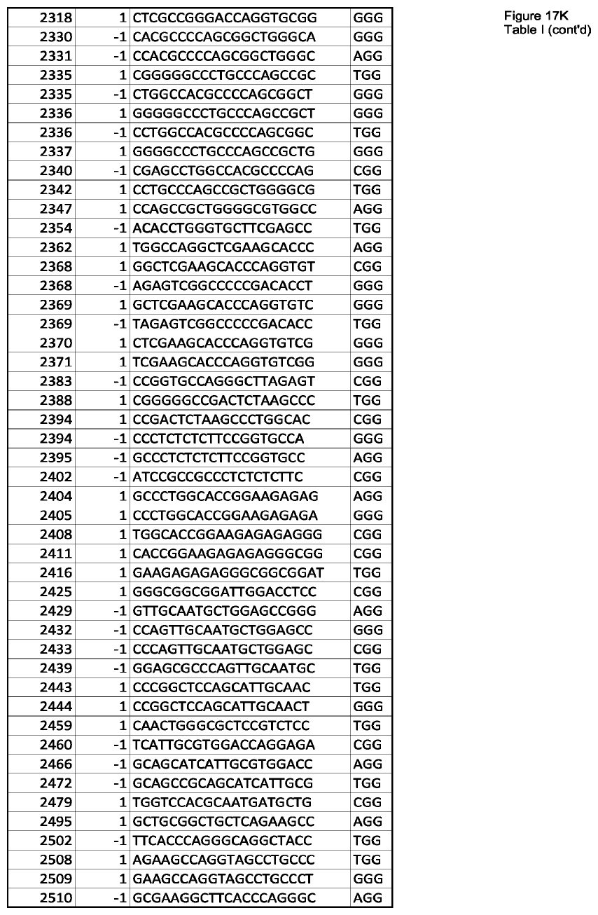

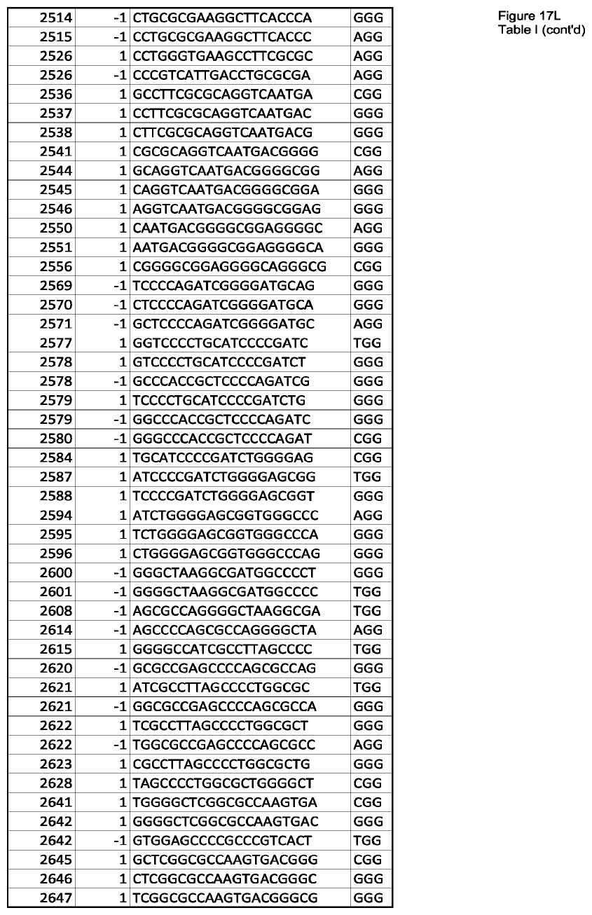

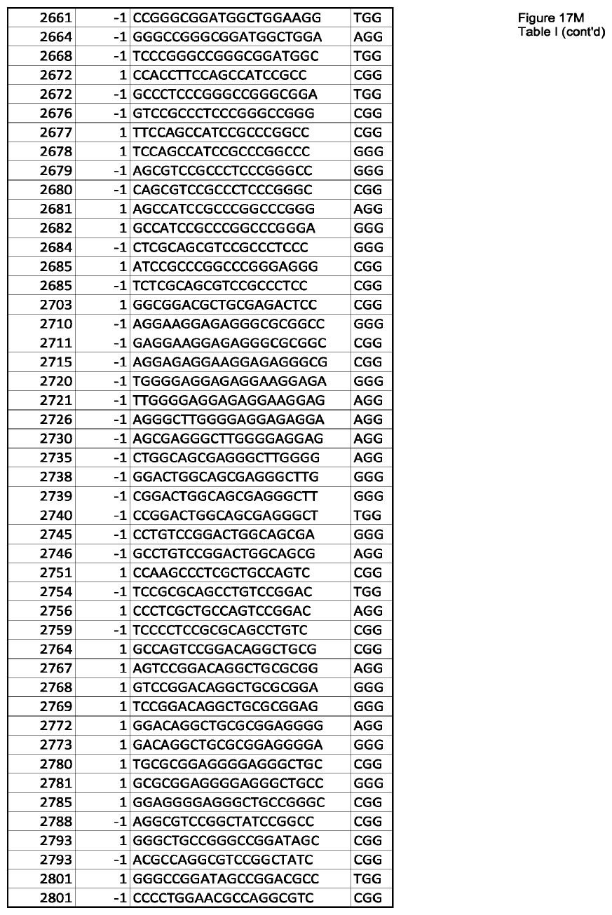

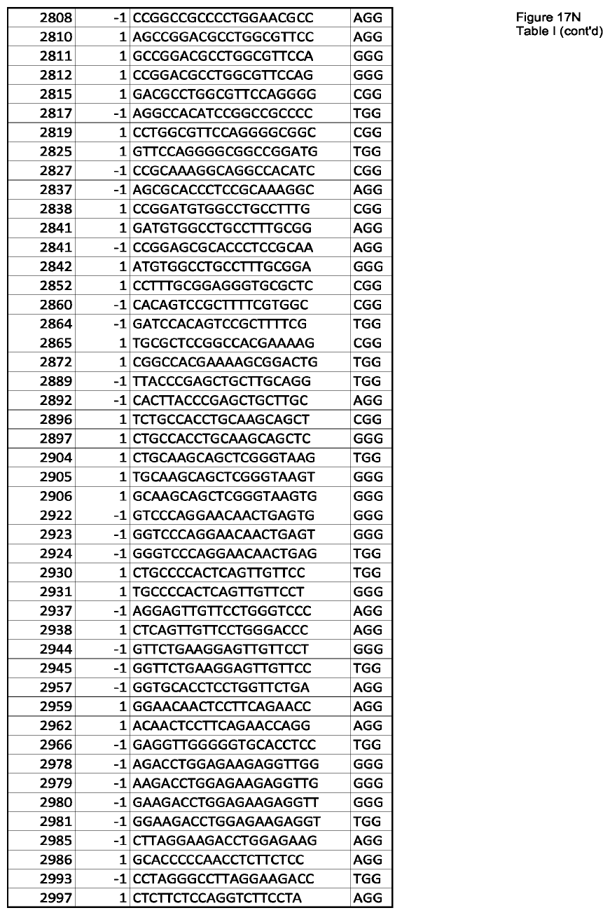

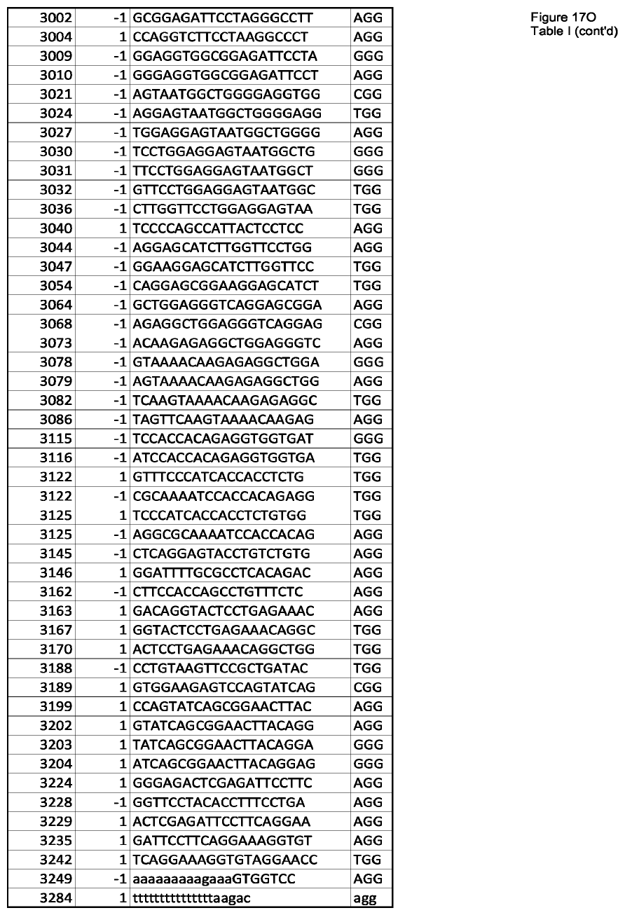

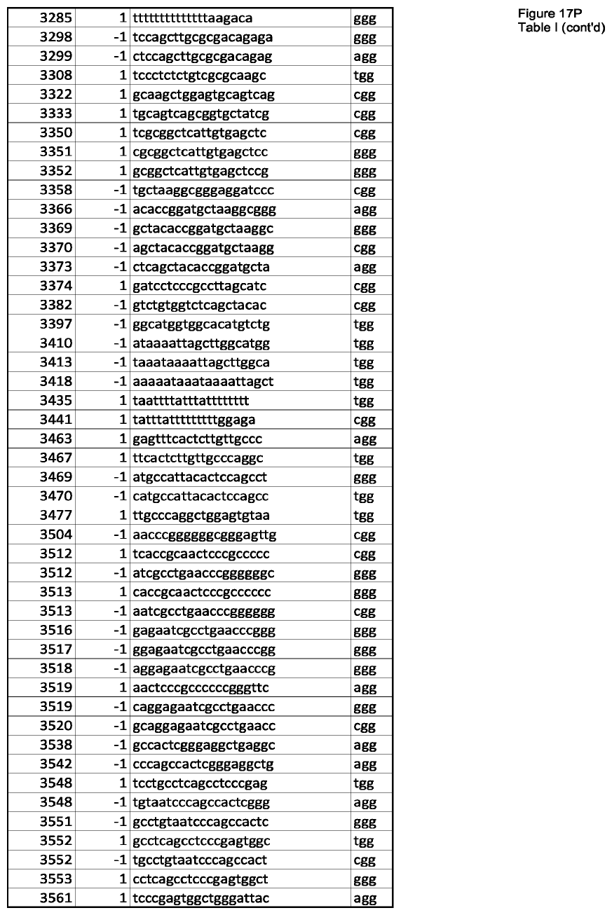

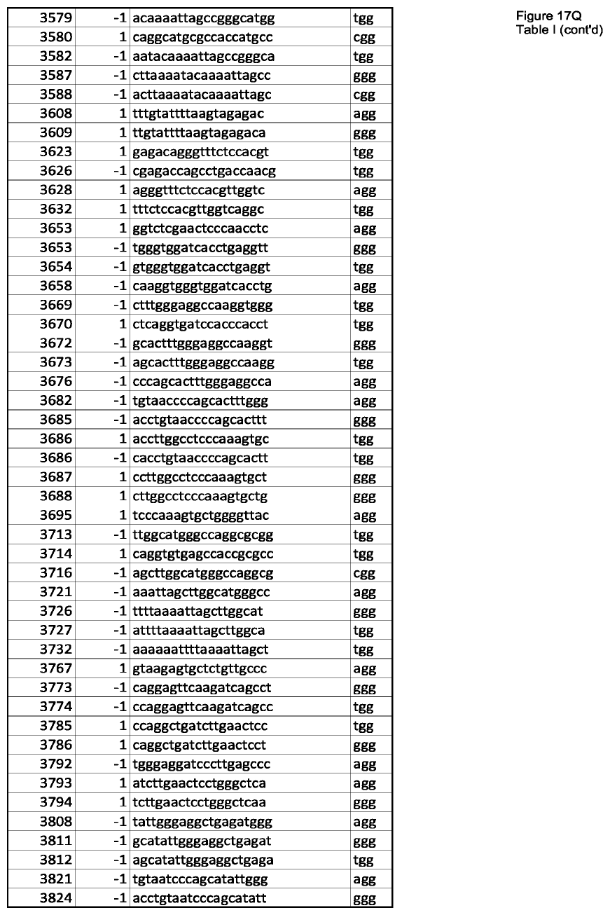

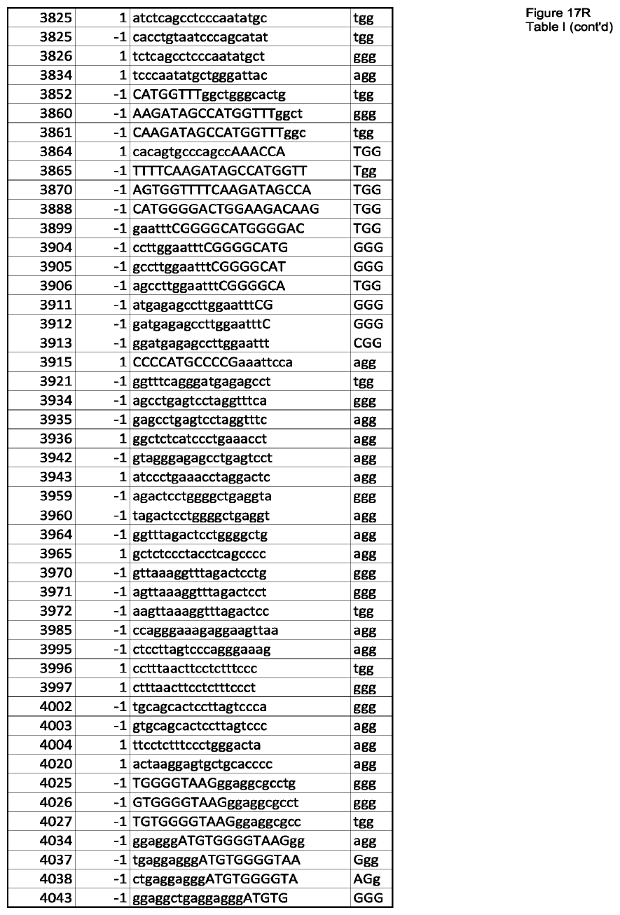

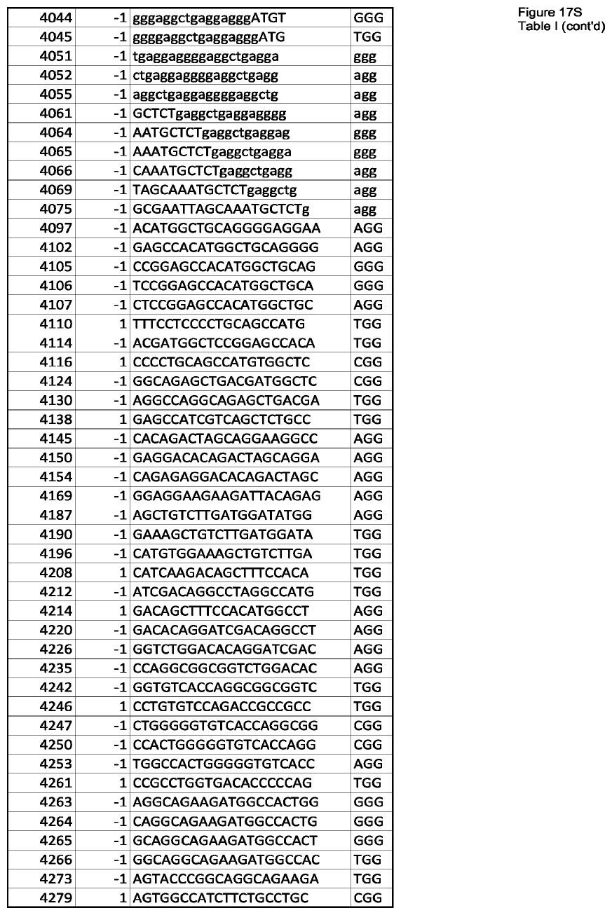

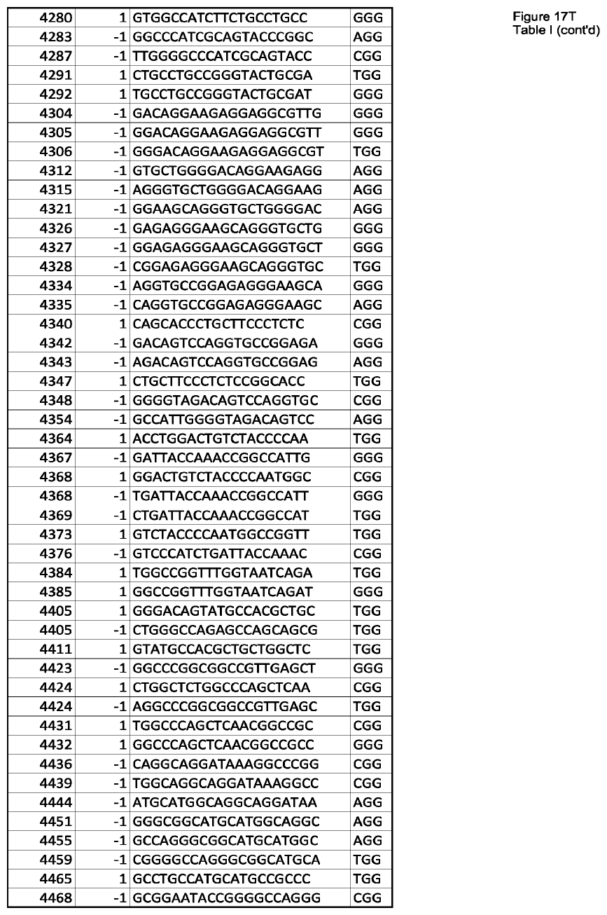

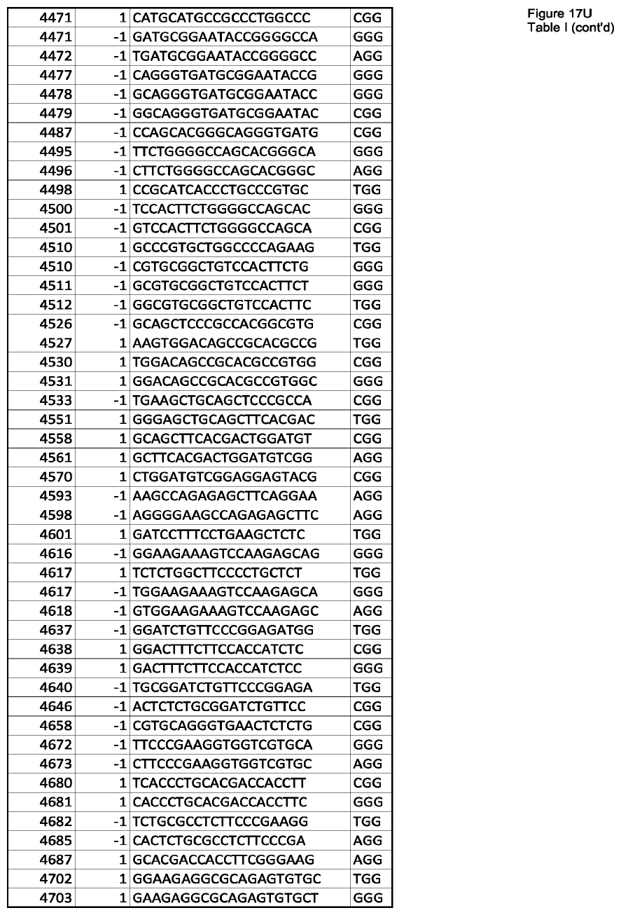

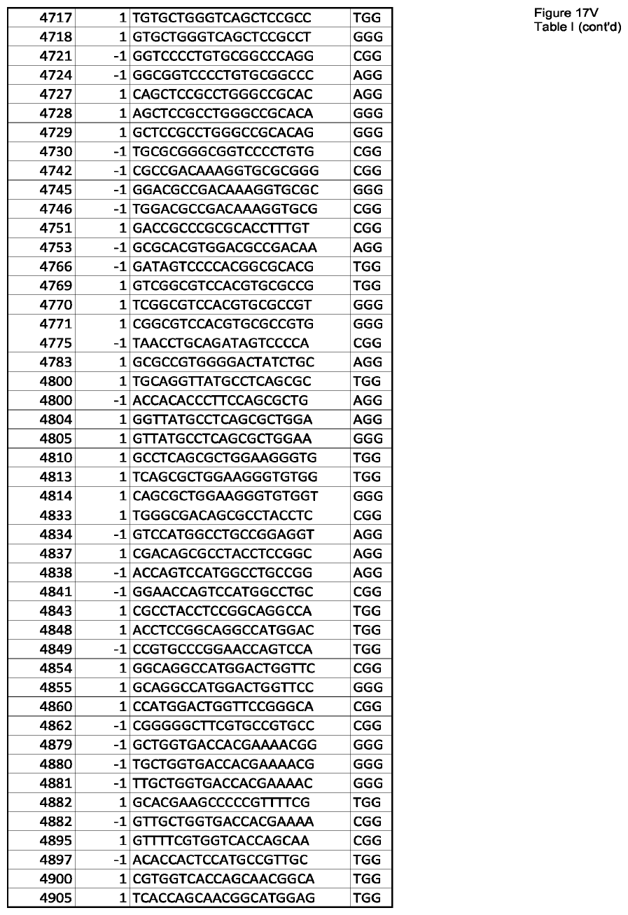

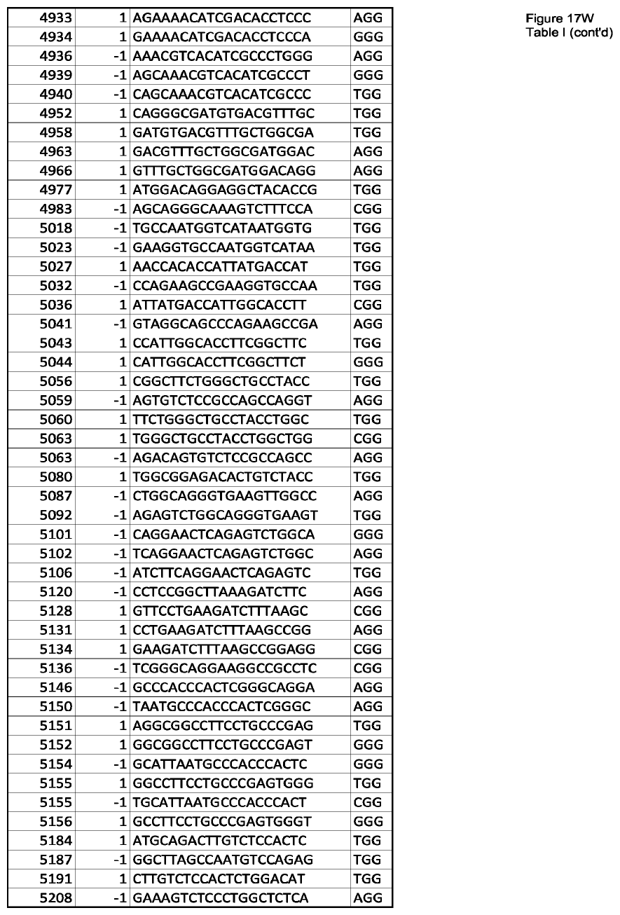

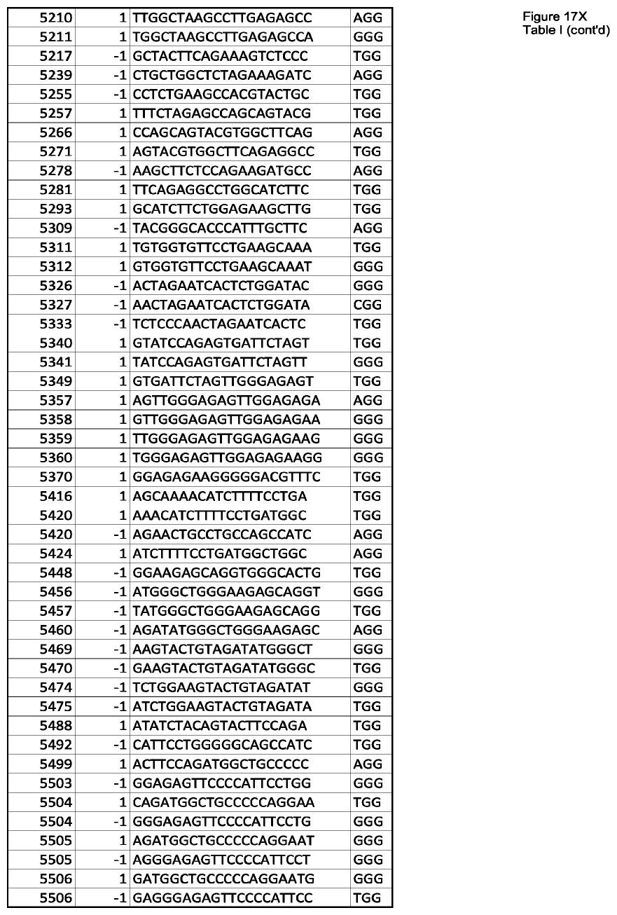

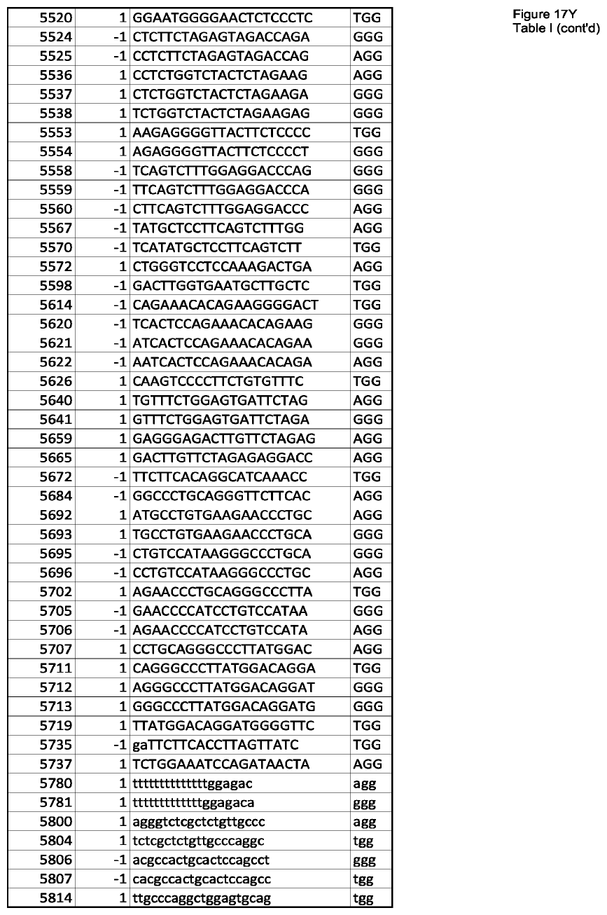

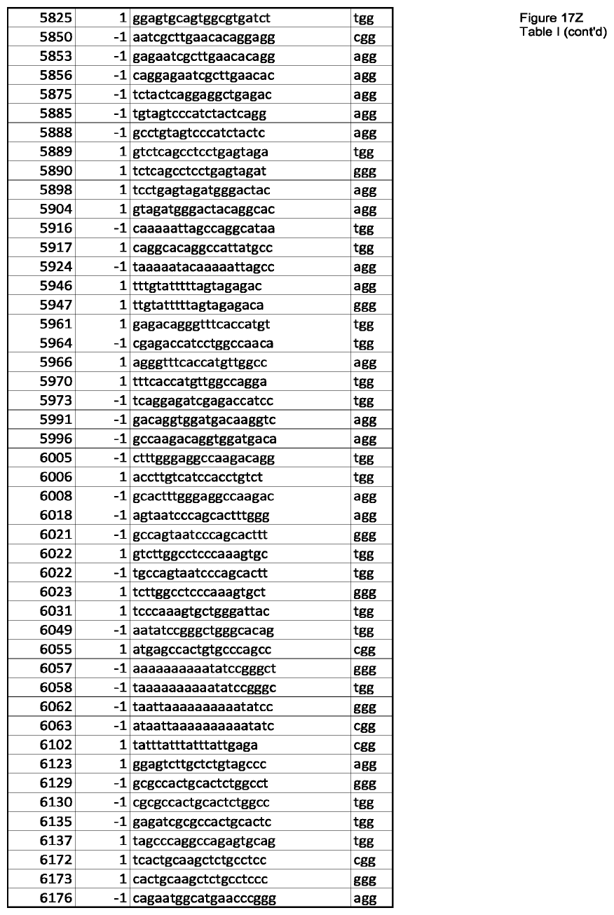

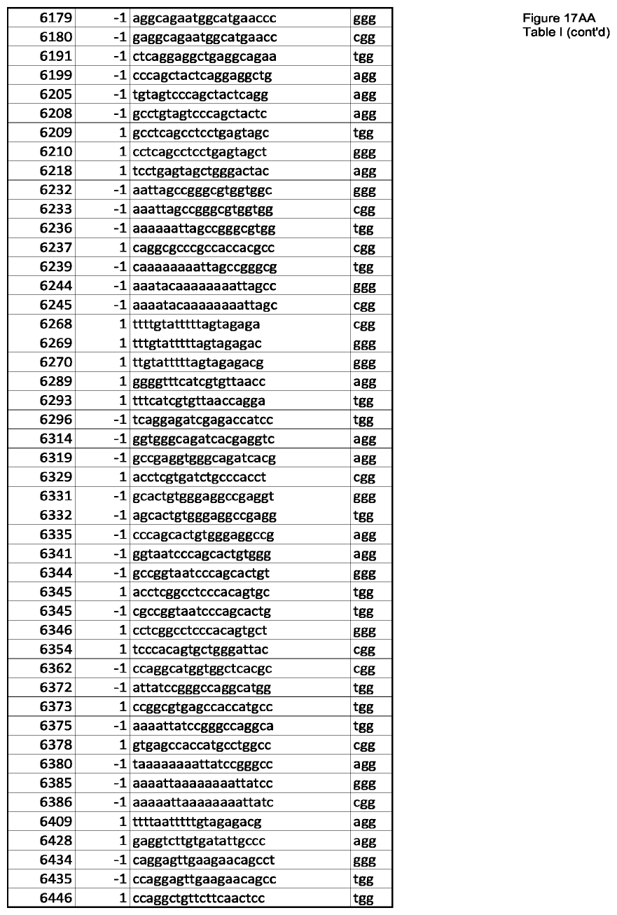

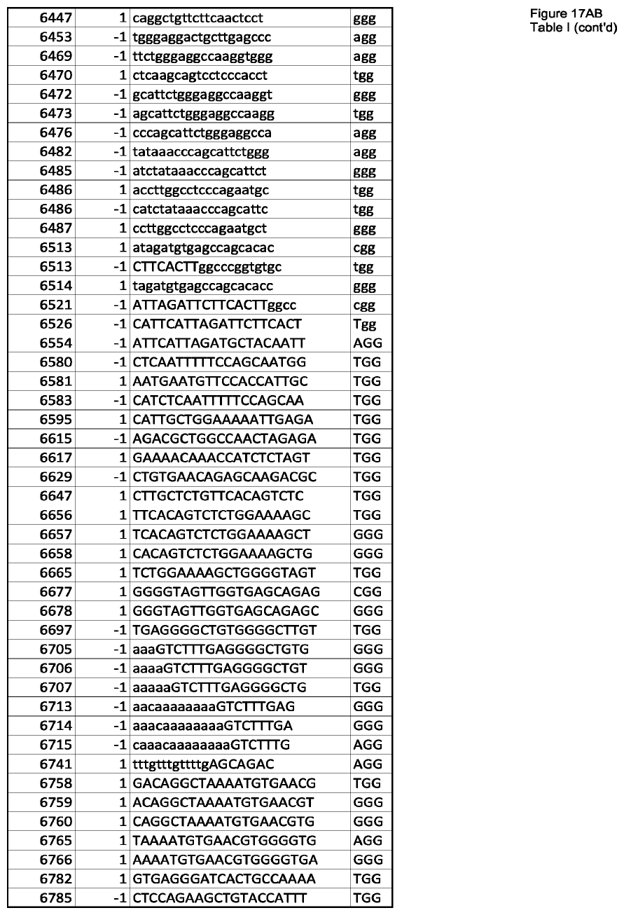

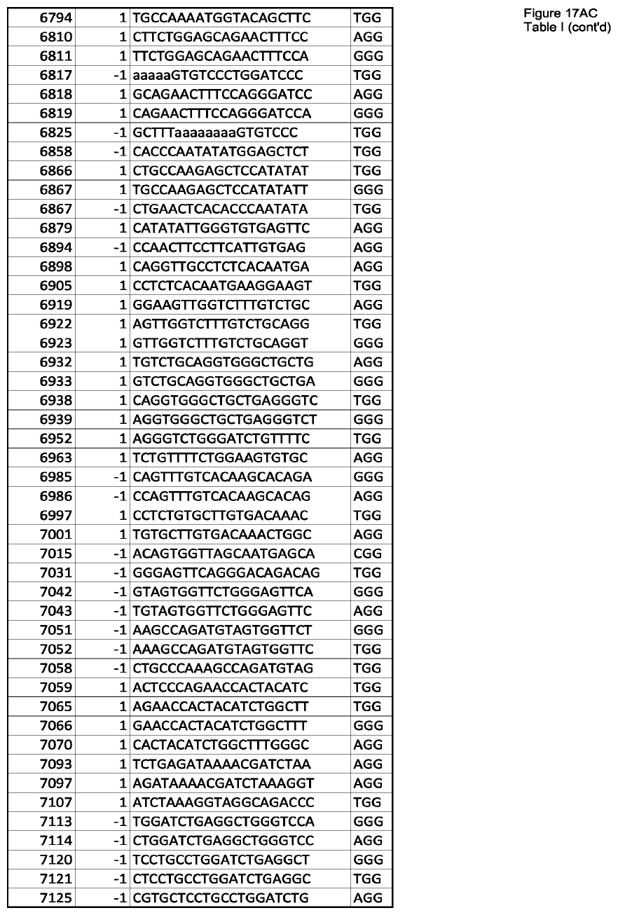

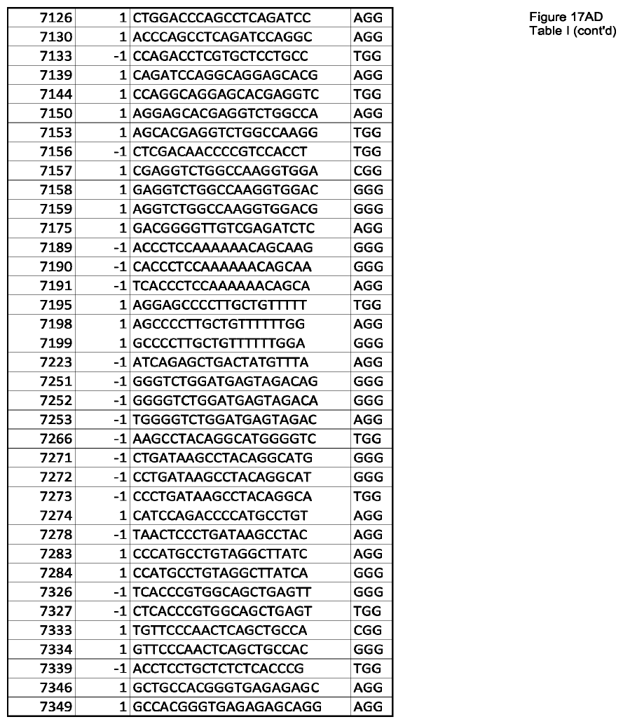

In some embodiments, hypoimmunogenicity is assayed using a number of techniques as exemplified in FIG. 13 and FIG. 15 of WO2018132783. These techniques include transplantation into allogeneic hosts and monitoring for hypoimmunogenic pluripotent cell growth (e.g. teratomas) that escape the host immune system. In some instances, hypoimmunogenic pluripotent cell derivatives are transduced to express luciferase and can then followed using bioluminescence imaging. Similarly, the T cell and/or B cell response of the host animal to such cells are tested to confirm that the cells do not cause an immune reaction in the host animal. T cell function is assessed by ELISpot, ELISA, FACS, PCR, or mass cytometry (CYTOF). B cell response or antibody response is assessed using FACS or Luminex. Additionally or alternatively, the cells may be assayed for their ability to avoid innate immune responses, e.g., NK cell killing, as is generally shown in FIGS. 14 and 15 of WO2018132783.

In some embodiments, the immunogenicity of the cells is evaluated using T cell immunoassays such as T cell proliferation assays, T cell activation assays, and T cell killing assays recognized by those skilled in the art. In some cases, the T cell proliferation assay includes pretreating the cells with interferon-gamma and coculturing the cells with labelled T cells and assaying the presence of the T cell population (or the proliferating T cell population) after a preselected amount of time. In some cases, the T cell activation assay includes coculturing T cells with the cells outlined herein and determining the expression levels of T cell activation markers in the T cells.

In vivo assays can be performed to assess the immunogenicity of the cells outlined herein. In some embodiments, the survival and immunogenicity of hypoimmunogenic cells is determined using an allogenic humanized immunodeficient mouse model. In some instances, the hypoimmunogenic pluripotent stem cells are transplanted into an allogenic humanized NSG-SGM3 mouse and assayed for cell rejection, cell survival, and teratoma formation. In some instances, grafted hypoimmunogenic pluripotent stem cells or differentiated cells thereof display long-term survival in the mouse model.

Additional techniques for determining immunogenicity including hypoimmunogenicity of the cells are described in, for example, Deuse et al., Nature Biotechnology, 2019, 37, 252-258 and Han et al., Proc Natl Acad Sci USA, 2019, 116(21), 10441-10446, the disclosures including the figures, figure legends, and description of methods are incorporated herein by reference in their entirety.

Similarly, the retention of pluripotency is tested in a number of ways. In one embodiment, pluripotency is assayed by the expression of certain pluripotency-specific factors as generally described herein and shown in FIG. 29 of WO2018132783. Additionally or alternatively, the pluripotent cells are differentiated into one or more cell types as an indication of pluripotency.

As will be appreciated by those in the art, the successful reduction of the MHC I function (HLA I when the cells are derived from human cells) in the pluripotent cells can be measured using techniques known in the art and as described below; for example, FACS techniques using labeled antibodies that bind the HLA complex; for example, using commercially available HLA-A, B, C antibodies that bind to the alpha chain of the human major histocompatibility HLA Class I antigens.

In addition, the cells can be tested to confirm that the HLA I complex is not expressed on the cell surface. This may be assayed by FACS analysis using antibodies to one or more HLA cell surface components as discussed above.

The successful reduction of the MHC II function (HLA II when the cells are derived from human cells) in the pluripotent cells or their derivatives can be measured using techniques known in the art such as Western blotting using antibodies to the protein, FACS techniques, RT-PCR techniques, etc.

In addition, the cells can be tested to confirm that the HLA II complex is not expressed on the cell surface. Again, this assay is done as is known in the art (See FIG. 21 of WO2018132783, for example) and generally is done using either Western Blots or FACS analysis based on commercial antibodies that bind to human HLA Class II HLA-DR, DP and most DQ antigens.

In addition to the reduction of HLA I and II (or MHC I and II), the hypoimmunogenic cells of the invention have a reduced susceptibility to macrophage phagocytosis and NK cell killing. The resulting hypoimmunogenic cells “escape” the immune macrophage and innate pathways due to the expression of one or more CD47 transgenes.

G. Maintenance of Hypoimmunogenic Pluripotent Stem Cells

Once the hypoimmunogenic pluripotent stem cells have been generated, they can be maintained an undifferentiated state as is known for maintaining iPSCs. For example, the cells can be cultured on matrigel using culture media that prevents differentiation and maintains pluripotency. In addition, they can be in culture medium under conditions to maintain pluripotency. Methods for culturing pluripotent stem cells are recognized in the art and are described, for example, in WO2016183041 and WO2018132783.

H. Differentiation of Hypoimmunogenic Pluripotent Stem Cells

The invention provides hypoimmunogenic pluripotent cells that are differentiated into different cell types for subsequent transplantation into subjects. As will be appreciated by those in the art, the methods for differentiation depend on the desired cell type using known techniques. The cells can be differentiated in suspension and then put into a gel matrix form, such as matrigel, gelatin, or fibrin/thrombin forms to facilitate cell survival. In some cases, differentiation is assayed as is known in the art, generally by evaluating the presence of cell-specific markers.

In some embodiments, the hypoimmunogenic pluripotent cells are differentiated into hepatocytes to address loss of the hepatocyte functioning or cirrhosis of the liver. There are a number of techniques that can be used to differentiate hypoimmunogenic pluripotent cells into hepatocytes; see for example Pettinato et al., doi:10.1038/spre32888, Snykers et al., Methods Mol Biol 698:305-314 (2011), Si-Tayeb et al, Hepatology 51:297-305 (2010) and Asgari et al., Stem Cell Rev (:493-504 (2013), all of which are hereby expressly incorporated by reference in their entirety and specifically for the methodologies and reagents for differentiation. Differentiation is assayed as is known in the art, generally by evaluating the presence of hepatocyte associated and/or specific markers, including, but not limited to, albumin, alpha fetoprotein, and fibrinogen. Differentiation can also be measured functionally, such as the metabolization of ammonia, LDL storage and uptake, ICG uptake and release and glycogen storage.

In some embodiments, the hypoimmunogenic pluripotent cells are differentiated into beta-like cells or islet organoids for transplantation to address type I diabetes mellitus (T1DM). Cell systems are a promising way to address T1DM, see, e.g., Ellis et al., doi/10.1038/nrgastro.2017.93, incorporated herein by reference. Additionally, Pagliuca et al. reports on the successful differentiation of β-cells from human iPSCs (see doi/10.106/j.cell.2014.09.040, hereby incorporated by reference in its entirety and in particular for the methods and reagents outlined there for the large-scale production of functional human β cells from human pluripotent stem cells). Furthermore, Vegas et al. shows the production of human β cells from human pluripotent stem cells followed by encapsulation to avoid immune rejection by the host; (doi:10.1038/nm.4030, hereby incorporated by reference in its entirety and in particular for the methods and reagents outlined there for the large-scale production of functional human (3 cells from human pluripotent stem cells).

Differentiation is assayed as is known in the art, generally by evaluating the presence of β cell associated or specific markers, including but not limited to, insulin. Differentiation can also be measured functionally, such as measuring glucose metabolism, see generally Muraro et al, doi:10.1016/j.cells.2016.09.002, hereby incorporated by reference in its entirety, and specifically for the biomarkers outlined there.

In some embodiments, the hypoimmunogenic pluripotent cells are differentiated into retinal pigment epithelium (RPE) to address sight-threatening diseases of the eye. Human pluripotent stem cells have been differentiated into RPE cells using the techniques outlined in Kamao et al., Stem Cell Reports 2014:2:205-18, hereby incorporated by reference in its entirety and in particular for the methods and reagents outlined there for the differentiation techniques and reagents; see also Mandai et al., doi:10.1056/NEJMoa1608368, also incorporated in its entirety for techniques for generating sheets of RPE cells and transplantation into patients.

Differentiation can be assayed as is known in the art, generally by evaluating the presence of RPE associated and/or specific markers or by measuring functionally. See for example Kamao et al., doi:10.1016/j.stemcr.2013.12.007, hereby incorporated by reference in its entirety and specifically for the markers outlined in the first paragraph of the results section.

In some embodiments, the hypoimmunogenic pluripotent cells are differentiated into cardiomyocytes to address cardiovascular diseases. Techniques are known in the art for the differentiation of hypoimmunogenic induced pluripotent stem cells to cardiomyoctes and discussed in the Examples. Differentiation can be assayed as is known in the art, generally by evaluating the presence of cardiomyocyte associated or specific markers or by measuring functionally; see for example Loh et al., doi:10.1016/j.cell.2016.06.001, hereby incorporated by reference in its entirety and specifically for the methods of differentiating stem cells including cardiomyocytes.

In some embodiments, the hypoimmunogenic pluripotent cells are differentiated into endothelial colony forming cells (ECFCs) to form new blood vessels to address peripheral arterial disease. Techniques to differentiate endothelial cells are known. See, e.g., Prasain et al., doi:10.1038/nbt.3048, incorporated by reference in its entirety and specifically for the methods and reagents for the generation of endothelial cells from human pluripotent stem cells, and also for transplantation techniques. Differentiation can be assayed as is known in the art, generally by evaluating the presence of endothelial cell associated or specific markers or by measuring functionally.

In some embodiments, the hypoimmunogenic pluripotent cells are differentiated into thyroid progenitor cells and thyroid follicular organoids that can secrete thyroid hormones to address autoimmune thyroiditis. Techniques to differentiate thyroid cells are known the art. See, e.g. Kurmann et al., doi:10.106/j.stem.2015.09.004, hereby expressly incorporated by reference in its entirety and specifically for the methods and reagents for the generation of thyroid cells from human pluripotent stem cells, and also for transplantation techniques. Differentiation can be assayed as is known in the art, generally by evaluating the presence of thyroid cell associated or specific markers or by measuring functionally.

Additional descriptions of methods for differentiating hypoimmunogenic pluripotent cells can be found, for example, in Deuse et al., Nature Biotechnology, 2019, 37, 252-258 and Han et al., Proc Natl Acad Sci USA, 2019, 116(21), 10441-10446.

I. Methods of Treatment

As will be appreciated by those in the art, the differentiated hypoimmunogenic pluripotent cell derivatives can be transplanted using techniques known in the art that depends on both the cell type and the ultimate use of these cells. In general, the cells of the invention can be transplanted either intravenously or by injection at particular locations in the patient. When transplanted at particular locations, the cells may be suspended in a gel matrix to prevent dispersion while they take hold.

In some embodiments, the cells or pharmaceutical composition thereof is administered to the subject systemically (e.g., orally, parenterally, subcutaneously, intravenously, intramuscularly, intraperitoneally) or locally. In some embodiments, the cells or pharmaceutical composition thereof is administered to the subject such that the cells reaches a target tissue selected from liver, lungs, heart, spleen, pancreas, gastrointestinal tract, kidney, testes, ovaries, brain, reproductive organs, central nervous system, peripheral nervous system, skeletal muscle, endothelium, inner ear, or eye. In some embodiments (e.g., wherein the subject has an autoimmune disease), the cells or pharmaceutical composition thereof is co-administered with an immunosuppressive agent, e.g., a glucocorticoid, cytostatic, antibody, or immunophilin modulator. In some embodiments (e.g., wherein the subject has a cancer or an infectious disease), the cells or pharmaceutical composition thereof is co-administered with an immunostimulatory agent, e.g., an adjuvant, interleukin, cytokine, or chemokine.

In some embodiments, the cells or pharmaceutical composition thereof is delivered ex-vivo to a tissue or organ, e.g., a human tissue or organ. In some embodiments, the composition is delivered to an ex vivo tissue that is in an injured state (e.g., from trauma, disease, hypoxia, ischemia or other damage).

In other embodiments, the cells or pharmaceutical composition thereof is used in vivo, i.e., to a human or mammal. In addition to humans, compositions described herein may also be used to similarly modulate the cell or tissue function or physiology of a variety of other organisms including but not limited to: farm or working animals (e.g., horses, cows, sheep, pigs, chickens etc.) and pet or zoo animals (e.g., cats, dogs, lizards, birds, lions, tigers, bears, etc.).

In some embodiments, the universal type O negative cells described herein are administered to a recipient subject determined to be ABO blood group type A, B, AB or O. In some embodiments, the universal type O negative cells are administered to a recipient subject determined to be Rh factor positive or Rh factor negative. In some embodiments, the universal type O negative cells are administered to a recipient subject determined to be ABO blood group type A, B, AB or O and Rh factor positive. In certain embodiments, the universal type O negative cells are administered to a recipient subject determined to be ABO blood group type A, B, AB or O and Rh factor negative. In some embodiments, the Bombay phenotype cells described herein are administered to a recipient subject determined to be ABO blood group type A, B, AB or O. In some embodiments, the Bombay phenotype cells are administered to a recipient subject determined to have the Bombay phenotype.

In some embodiments, the cells are not targeted by the subject's immune system.

In some embodiments, the cells described are administered alone or formulated as a pharmaceutical composition. The administration of a pharmaceutical composition may be by way of transdermal or parenteral (including intravenous, intratumoral, intraperitoneal, intramuscular, intracavity, and subcutaneous) administration. In certain instances, the administration further comprises a bolus or by continuous perfusion.

In some embodiments, the cells or composition thereof co-administered with an additional agent, e.g., a therapeutic agent, to a recipient subject. In some embodiments, the co-administered therapeutic agent is an immunosuppressive agent, e.g., a glucocorticoid (e.g., dexamethasone), cytostatic (e.g., methotrexate), antibody (e.g., muromonab-CD3), or immunophilin modulator (e.g., ciclosporin or rapamycin). In some embodiments, the immunosuppressive agent decreases immune mediated clearance of cells. In some embodiments, the cells or composition thereof is co-administered with an immunostimulatory agent, e.g., an adjuvant, an interleukin, a cytokine, or a chemokine.

In some embodiments, a therapeutically effective amount of the cells or a composition thereof is administered to treat a disease. In some embodiments, a cell composition is administered with a pharmaceutically acceptable carrier. In some embodiments, the cells are any one of the cells described herein including differentiated cells derived from a stem cell.

### IV. EXAMPLES

### Example 1: Engineering of Universal Histo-Blood Type Cells

A. Cell Culture

The Gibco Human Episomal iPSC line (ThermoFisher Scientific, A1895) is obtained and cultured in StemFlex Medium (ThermoFisher Scientific, A3349401) and grown on 0.5 μg/cm² Vitronectin Recombinant Human Protein (ThermoFisher Scientific, A31804) coated plates or flasks. Cells are passaged when they reach ˜90-95% confluency. To passage, the cells are first washed 2× with DPBS −/− (Life Technologies, 14190250). Accutase (ThermoFisher Scientific A1110501) is added and cells are incubated at 37° C. for 5 minutes, or until cells detach by gently rocking the plate or flask. Cells are rigorously resuspended to facilitate single cell suspension, and then washed with DMEM/F12 (ThermoFisher Scientific, 11330057) before spinning down for 3 minutes at 250 g. The supernatant is removed, and the cell pellet is resuspended in StemFlex Medium+10 μM Y-27632 (Fisher Scientific, 12-545-0). Cells are then counted and seeded at the appropriate density on a new Vitronectin coated plates or flasks in StemFlex Medium+10 uM Y-27632. Media is replaced the next day with StemFlex media minus Y-27632, and exchanged every two days until cells reach 90-95% confluency.

B. Guide RNA and HDR Template Design

Synthetic guide RNAs (sgRNAs) for ABO, RHD, FUT1 are designed using the Benchling CRISPR Guide RNA Design tool with genomic sequences from ENSEMBL: ABO (ENSG00000175164), RHD (ENSG00000187010), and FUT1 (ENSG00000174951). For ABO, three gRNA sequences targeting exon6/7 are utilized in combination with homology directed repair (HDR) templates encoding c.258delG and 100 bases of homologous sequence upstream and downstream. Guide RNA directed to exon 6-7 gRNA1 target (5′-GCCAGCCAAGGGGTACCACG-3′; SEQ ID NO:24) is paired with single-stranded repair template 1 (5′-TGGGGGCGGCCGTGTGCCAGAGGCGCATGTGGGTGGCACCCTGCCAGCTCCATGTG ACCGCACGCCTCTCTCCATGTGCAGTAGGAAGGATGTTCTCGTGTACCCCTTGGCTG GCTCCCATTGTCTGGGAGGGCACATTCAACATCGACATCCTCAACGAGCAGTTCAG GCTCCAGAACACCACCATTGGGTTAACTGTG-3′; SEQ ID NO:25). Guide RNA directed to exon 6-7 gRNA2 target (5′-GATGTCCTCGTGGTACCCCT-3′; SEQ ID NO:26) is paired with single-stranded repair template 2 (5′-TGGGGGCGGCCGTGTGCCAGAGGCGCATGTGGGTGGCACCCTGCCAGCTCCATGTG ACCGCACGCCTCTCTCCATGTGCAGTAGGAAGGATGTCCTCGTAAACCCCTTGGCTG GCTCCCATTGTCTGGGAGGGCACATTCAACATCGACATCCTCAACGAGCAGTTCAG GCTCCAGAACACCACCATTGGGTTAACTGTG-3′; SEQ ID NO:27). Guide RNA directed to exon 6-7 gRNA3 target (5′-AATGTGCCCTCCCAGACAAT-3′; SEQ ID NO:28) is paired with single-stranded repair template 3 (5′-TGGGGGCGGCCGTGTGCCAGAGGCGCATGTGGGTGGCACCCTGCCAGCTCCATGTG ACCGCACGCCTCTCTCCATGTGCAGTAGGAAGGATGTCCTCGTGTACCCCTTGGCTG GCTCCCATTGTTTGGGAGGGCACTTTCAACATCGACATCCTCAACGAGCAGTTCAGG CTCCAGAACACCACCATTGGGTTAACTGTG-3′; SEQ ID NO:29). Alternatively, Guide RNA directed to exon 1 gRNA target (5′-GGCCAGCGTCCGCAACACCT-3′; SEQ ID NO:30) or guide RNA directed to exon 2 gRNA target (5′-GGATCATAGGTCGAAGTGCG-3′; SEQ ID NO:31) are used to introduce double-stranded breaks (DSBs) into the early codon exons 1 and 2. For RHD, two gRNA sequences targeting exon 2 target sequence (5′-CACCGACAAAGCACTCATGG-3′; SEQ ID NO:32) and exon 3 target sequence (5′-TGGCCAAGATCTGACCGTGA-3′; SEQ ID NO:33) are used to introduce DSBs into the early codon exons 1 and 2. Alternatively, a gRNA targeting the 5′ UTR target sequence (5′-TGGTTGTGCTGGCCTCTCTA-3′; SEQ ID NO:34) in combination with a gRNA targeting exon 8 (5′-GGAGGCGCTGCGGTTCCTAC-3′; SEQ ID NO:35) are used to remove the entire coding sequence of the gene from the genome. For FUT1, two gRNA sequences targeting exon 4 (the only coding exon) include guide RNA directed to exon 4 gRNA1 target (5′GGTCTGGACACAGGATCGAC-3′; SEQ ID NO:36) and guide RNA directed to exon 4 gRNA2 target (5′-GATTACCAAACCGGCCATTG-3′; SEQ ID NO:37) and are used to induce DSBs into the coding sequence of the gene.

C. Cell Engineering

gRNAs targeting either ABO or FUT1 are complexed with recombinant SpyFi Cas9 protein procured from Aldevron for 15′ at room temperature in nuclease free water. Cells are harvested in STEMFLEX and vitronectin using ACCUTASE cell detachment solution, pelleted and resuspended in Lonza NUCLEOFECTOR buffer P3+RNP complex and transferred to a Lonza NUCLEOCUVETTE vessel. Cells are electroporated with the Lonza 4D-NUCLEOFECTOR core unit using the CA-137 program and supplemented with STEMFLEX medium with CLONER supplement. Cells are transferred to 2 vitronectin coated wells in a 24-well plate containing STEMFLEX medium with CLONER supplement. Cells are expanded and then electroporated again with an RNP complex containing a gRNA targeting the RHD gene. Cells are stained with antibodies against the A antigen and Rh antigen and the negative population of cells are sorted into single wells of a 96-well plate by FACS. Individual clones are then stained again to verify that the cells are O type Rh negative or Bombay type Rh negative, and this is confirmed by genomic analysis.

D. Genomics Assays to Characterize ABO, RHD and FUT1 Gene Editing

Amplicons containing the targeted regions of ABO, RHD, FUT1 are produced using primers specific to these sites and the sequences are sequenced using an Illumina NextSeq sequencing platform. The results are aligned to the human genome and analyzed for insertions and deletions (indels).

E. Phenotypic Assays to Characterize Knock-Out (KO) of ABO, RHD and FUT1 Genes

Single cell cloning of gene edited cells is performed using the Hana single cell dispenser (Namocell) into 96 well plates. Single cell deposition confirmation and clonal cell outgrowth is monitored using the CELIGO imaging cyometer (Nexcelcom). The single cells clones are passaged and expanded into the following three cohorts: 1. Cell maintenance and banking; 2. Genomic characterization by Next Generation Sequencing (NGS); and 3. Phenotypic characterization.

F. Phenotypic Characterization by Immunofluorescence

TABLE 6
Reagents for phenotypic characterization

| Description | Catalog Number | Supplier | Dilution |
|---|---|---|---|
| Anti-RhD antibody | ab222792 | Abcam | 1:50 |
| Anti-Blood Group AB antigen antibody | ab24223 | abcam | 1:400 |
| Blood Group Antigen H (O) Type 1 Monoclonal | 53-9810-82 | ThermoFisher | 1:50 |
| Antibody (17-206), Alexa Fluor 488, eBioscience ™ | | | |
| Anti-Blood Group H ab antigen antibody | ab24213 | abcam | 1:50 |
| FUT1 Polyclonal Antibody | PA5-13515 | ThermoFisher | 1:50 |
| Anti-Galactoside 2-alpha-L-fucosyltransferase 1 | ab198712 | Abcam | 1:50-1:200 |
| antibody (FUT1) | | | |
| Goat anti-mouse secondary ab AF488 | A-11029 | ThermoFisher | 1:500 |
| Goat anti-rabbit secondary ab AF647 | A-21244 | ThermoFisher | 1:500 |
| Hoechst 33342 | H3570 | ThermoFisher | 1:2000 |
| Normal Donkey Serum | 566460 | Sigma | 5% |
| 16% Formaldehyde (w/v), Methanol Free | 28908 | ThermoFisher | 1:4 |
| Phosphate Buffered Saline (PBS) | 10010023 | ThermoFisher | |

Cells are washed with PBS and fixed in 4% paraformaldehyde in PBS for 15 minutes. The cells are then washed and blocked in 5% donkey serum in PBS/TWEEN-20 for 15 minutes at room temperature. Cells are rinsed three times in PBS prior to the application of primary antibodies. Antibodies are diluted in blocking buffer (5% donkey serum/PBS) according to the specified dilution in Table 6 above overnight at 4 C. Cells are then rinsed three time in PBS and Alexa-488 and Alexa-647 conjugated antibodies are diluted to 1:500 in PBS and added to the cells for 30 minutes at room temperature. Antibodies conjugated to Alexa Fluor 488 are incubated overnight and do not require a secondary antibody. Cells are washed three times and Hoechst is diluted in PBS and added to the cells. Cells are ready for imaging on the Cytation5 cell imaging reader. Wild type (WT) cells are maintained throughout the workflow and gene KO is confirmed relative to WT cell expression levels.

G. Functional Assays to Validate Knockout of ABO, RHD, and FUT1

Human serum from an O negative individuals is obtained from a vendor. Complement mediated cell killing assays are performed on the XCelligence® SP platform (ACEA BioSciences). 96-well E-plates (ACEA BioSciences) are coated with collagen (Sigma-Aldrich) and universal type O Rh negative or type Bombay Rh negative iPSCs are plated in 100 μl cell-specific media. After the Cell Index value reaches 0.7, human O negative serum is added. As a negative control, heat inactivated serum is used. Data are standardized and analyzed with the RTCA software (ACEA).

### Example 2: Differentiation of RUES2 Embryonic Stem Cells that are B Positive Cells

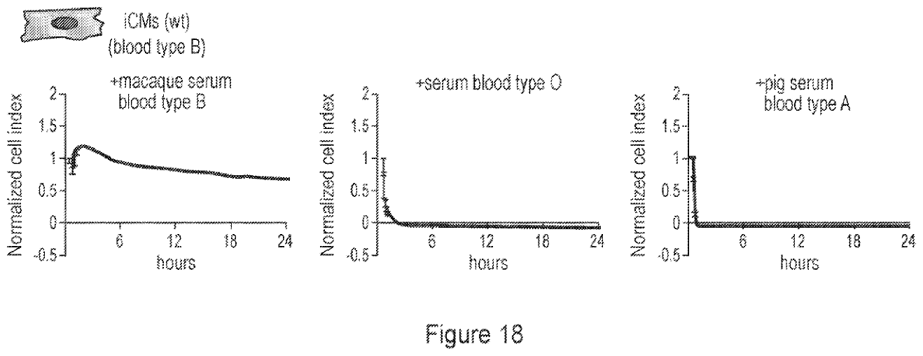

Human RUES2 embryonic stem cells (Lacoste et al., Cell Stem Cell 5 (3): 332-342 (2009); hPSCReg ID: RUESe002-A) were differentiated into cardiomyocyte-like cells. The resulting RUES2-derived cells survived when incubated with blood type B macaque serum but the cells were killed when incubated with human blood type O serum or pig blood type A serum as measured by a real-time cell analysis assay (XCelligence®; FIG. 18). This result indicates that RUES2 and differentiated cardiomyocytes derived therefrom are blood type B.

Additionally, undifferentiated RUES2 cells were characterized by PCR assays for blood type (see, e.g., Mohamed et al., Blood Res. 15 (4): 274-278 (20016)) and Rh status. Specifically, a pair of oligonucleotide primers that recognize a sequence in exon 4 and exon 5, respectively, of Rh D and Rh CcEe are synthesized and used on genomic DNA of Rh D phenotyped cells. After an initial cycle of denaturation at 99° C. for 5 minutes, 35 cycles were performed consisting of 1 minute at 95° C. of denaturation, 1.5 minutes at 55° C. of primer annealing, and 2.5 minutes at 72° C. of extension, followed by a final cycle of 9 minutes at 72° C. The Rh CcEe gene is distinguished from the Rh D gene, since in the Rh D gene there is a deletion in intron 4 between exon 4 and 5, resulting in a DNA fragment that is 600 bp smaller than that obtained from the CcEe gene. With primers A9 and A6, a PCR product of approximately 1200 bp was derived from the CcEe gene, whereas the smaller PCR product of approximately 600 bp, derived from the Rh D gene, was lacking when the donor was D negative. PCR products were size-separated on 2%-agarose gels and visualized by UV illumination after ethidium bromide staining. The results confirmed that the RUES2 cells were blood type B and Rh positive.

Exemplary Oligonucleotide Primer Sequences:

A6.
(SEQ ID NO: 21)
5′ TGACCCTGAGATGGCTGT 3′ (antisense)
A9.
(SEQ ID NO: 22)
5′ ACGATACCCAGTTTGTCT 3′ (sense)

Additional techniques for determining Rh status of the cells using PCR assays are described in, for example, Simsek et al., Blood, 1995, 85(10), 2975-2980 and Arce et al., Blood, 1993, 82(651). the disclosures including the figures, figure legends, and description of methods are incorporated herein by reference in their entirety.

### Example 3: Genome Modification of Human ABO Gene

Human episomal iPSC (Gibco) are blood type A-positive. The cells were edited to O-positive by creation of indels in the ABO gene. Guide RNA UCUCUCCAUGUGCAGUAGGA (SEQ ID NO:17) was complexed with S. pyogenes (sp) as9 to form a ribonucleoprotein (RNP). The RNP was then delivered to the cells via electroporation. The cells were then recovered for two days before evaluation of the edits. Edits of the ABO gene were assessed by PCR-amplification of the edited site and Sanger sequencing of the amplicons.

The edited pool was used to seed single cells for clonal expansion by single cell dilution. The resulting clones were verified using Sanger sequencing and two homozygous ABO KO clones were selected. The two selected clones were a +1 indel and a +2 indel. The resulting cells are functionally assayed to be blood type O-positive.

### Example 4: Genome Modification of Human iPSC from A+ to O+

Human episomal iPSC (Gibco) were edited from A positive (A+) to O positive (O+) by introduction of the ABO 258delG mutation in exon 6 by homology directed repair (HDR). Guide RNA CAGUAGGAAGGAUGUCCUCG (SEQ ID NO:18) was complexed with sp Cas9 to form a ribonucleoprotein (RNP). The RNP and donor template (below) were then delivered to the cells via electroporation. The cells were then recovered for two days before evaluation of the edits. Edits of the ABO gene were assessed by PCR-amplification of the edited site and Sanger sequencing of the amplicons.

Donor Template:

(SEQ ID NO: 19)
CTCCATGTGACCGCACGCCTCTCTCCATGTGCAGTAGGAAGGATGTCCT
CGTGTACCCCTTGGCTGGCTCCCATTGTCTGGGAGGGCACATTCAACAT
CGAC.

The edited pool was used to seed single cells for clonal expansion by single cell dilution. The resulting clones were verified using Sanger sequencing and two homozygous ABO 258delG mutation clones were selected. The resulting cells are functionally assayed to be blood type O-positive.

### Example 5: Genome Modification of Human FUT1 Gene

Human episomal iPSC (Gibco) were edited from A-positive to Bombay phenotype, Rh positive by creation of indels in the FUT1 gene. Guide RNA CUGGAUGUCGGAGGAGUACG (SEQ ID NO:23) was complexed with S. pyogenes (sp) Cas9 to form a ribonucleoprotein (RNP). The RNP was then delivered to the cells via electroporation. The cells were then recovered for two days before evaluation of the edits. Edits of the FUT1 gene were assessed by PCR-amplification of the edited site and Sanger sequencing of the amplicons.

The edited pool was used to seed single cells for clonal expansion by single cell dilution. The resulting clones were verified using Sanger sequencing and two homozygous FUT1 KO clones were selected. The resulting cells are functionally assayed to be Bombay phenotype, Rh positive.

All headings and section designations are used for clarity and reference purposes only and are not to be considered limiting in any way. For example, those of skill in the art will appreciate the usefulness of combining various aspects from different headings and sections as appropriate according to the spirit and scope of the invention described herein.

All references cited herein are hereby incorporated by reference herein in their entireties and for all purposes to the same extent as if each individual publication or patent or patent application was specifically and individually indicated to be incorporated by reference in its entirety for all purposes.

Many modifications and variations of this application can be made without departing from its spirit and scope, as will be apparent to those skilled in the art. The specific embodiments and examples described herein are offered by way of example only, and the application is to be limited only by the terms of the appended claims, along with the full scope of equivalents to which the claims are entitled.

1-174. (canceled)

175. An engineered cell in which expression of a blood group antigen gene is partially or fully inactivated and comprises one or more modifications, wherein the one or more modifications:
(a) inactivate or disrupt one or more alleles of:
(i) one or more major histocompatibility class I (MHC-I) molecules and/or one or more molecules that regulate expression of the one or more MHC-I molecules, and/or
(ii) one or more MHC class II (MHC-II) molecules and/or one or more molecules that regulate expression of the one or more MHC-II molecules, and/or
(b) increase expression of one or more tolerogenic factors,
wherein the increased expression of (b) is relative to an isolated cell that does not comprise the modifications.

176. The engineered cell of claim 175, wherein the blood group antigen gene is an ABO gene that is partially or fully inactivated by:
(a) insertion of an exon 6 258delG variation of the ABO gene,
(b) an insertion within exon 1, 2, 6, 7, or 8 of the ABO gene, or
(c) a deletion within exon 1, 2, 6, 7, or 8 of the ABO gene.

177. The engineered cell of claim 175, wherein the blood group antigen gene is a FUT1 gene that is partially or fully inactivated by an insertion or a deletion within exon 2 or exon 4.

178. The engineered cell of claim 175, wherein the blood group antigen gene is an RHD gene that is partially or fully inactivated by:
(a) an insertion or a deletion in a 5′ untranslated region (UTR) of the RHD gene, or
(b) an insertion of a deletion within exon 1, 2, 3, 4, 5, 6, 7, or 8 of the RHD gene.

179. The engineered cell of claim 175, wherein the cell is an Rh negative cell.

180. The engineered cell of claim 175, wherein the cell has a type O or Bombay phenotype.

181. The engineered cell of claim 175, wherein the cell is homozygous for the partial or full inactivation of the ABO gene.

182. The engineered cell of claim 175, wherein the cell is a human cell.

183. The engineered cell of claim 175, wherein the cell is selected from the group consisting of an induced pluripotent stem cell, an embryonic stem cell, an adult stem cell, and a differentiated cell.

184. The engineered cell of claim 176, wherein the ABO gene that is partially or fully inactivated is generated using a CRISPR/Cas gene editing system,
wherein the CRISPR/Cas gene editing system comprises a guide RNA targeting the ABO gene,
wherein the guide RNA targets a sequence selected from Table 1, Table A, Table B or Table C.

185. The engineered cell of claim 184, wherein the guide RNA targets a sequence comprising SEQ ID NO: 1 or SEQ ID NO: 2.

186. The engineered cell of claim 184, wherein the guide RNA targets a sequence comprising SEQ ID NO: 3, 4, 5, 15 or 16.

187. The engineered cell of claim 184, wherein the guide RNA comprises a nucleotide sequence of SEQ ID NO: 17 or SEQ ID NO: 18.

188. The engineered cell of claim 176, wherein the ABO gene is partially or fully inactivated by an insertion or deletion within ACACCT of exon 1; AGTGCG of exon 2; or CAAGCGC, GGCGCA, TGGGCG, ACCACG, GCCGCA, CCACGT, TGCCGT, TACTCG, ACTCGG, GAGCGC, GCCTGG, GTCCTT, TCACGC, TGGACG, TGGTCG, GCTGGC, CACGCG, AGGTGA, or GCATCG of exon 8 of the ABO gene.

189. The engineered cell of claim 176, wherein the exon 6 258delG variation of the ABO gene is generated using a CRISPR/Cas gene editing system,
wherein the CRISPR/Cas gene editing system comprises:
a guide RNA targeting the ABO gene that targets any one of SEQ ID NOs: 3, 4, 5, or 18, and
a homology directed repair (HDR) donor template comprising:
a sequence encoding the 258delG variation, a PAM sequence, a spacer region, and two homology arms,
wherein each homology arm comprises about 10 to about 1,000 nucleotides homologous to the ABO gene.

190. The engineered cell of claim 176, wherein the exon 6 258delG variation of the ABO gene is generated using a CRISPR/Cas gene editing system,
wherein the CRISPR/Cas gene editing system comprises:
a guide RNA targeting the ABO gene that targets any one of SEQ ID Nos: 3, 4, 5, or 18, and
an HDR donor template comprising a sequence of any one of SEQ ID NOs: 6, 7, 8, or 19.

191. The engineered cell of claim 176, wherein the insertion of an exon 6 258delG variation of the ABO gene is a homozygous variation such that the cell is a type O cell.

192. The engineered cell of claim 175, wherein the one or more molecules that regulate expression of the one or more MHC-I molecules regulate cell surface protein expression of the one or more MHC-I molecules.

193. The engineered cell of claim 175, wherein the one or more molecules that regulate expression of the one or more MHC-II molecules regulate cell surface protein expression of the one or more MHC-II molecules.

194. The engineered cell of claim 175, wherein the one or more modifications reduce expression of:
one or more MHC-I molecules;
one or more MHC-II molecules; or
one or more MHC-I molecules and one or more MHC-II molecules.

195. The engineered cell of claim 175, wherein the one or more modifications reduce expression of one or more molecules selected from the group consisting of B2M, TAP I, NLRC5, CIITA, HLA-A, HLA-B, HLA-C, HLA-DP, HLA-DQ, HLA-DR, HLA-DM, HLA-DO, RFX5, RFXANK, RFXAP, NFY-A, NFY-B, NFY-C, IRFI, and any combination thereof.

196. The engineered cell of claim 175, wherein the one or more modifications reduce cell surface protein expression of the one or more MHC-I molecules.

197. The engineered cell of claim 175, wherein the one or more modifications reduce cell surface trafficking of the one or more MHC-I molecules.

198. The engineered cell of claim 175, wherein the one or more modifications reduce expression of B2M.

199. The engineered cell of claim 175, wherein the one or more modifications reduce expression of HLA-A, HLA-B, and/or HLA-C.

200. The engineered cell of claim 175, wherein the one or more modifications reduce cell surface protein expression of the one or more MHC-II molecules.

201. The engineered cell of claim 175, wherein the one or more modifications reduce cell surface trafficking of the one or more MHC-II molecules.

202. The engineered cell of claim 175, wherein the one or more modifications reduce expression of CIITA.

203. The engineered cell of claim 175, wherein the one or more modifications reduce expression of HLA-DM, HLA-DO, HLA-DP, HLA-DQ, and/or HLA-DR.

204. The engineered cell of claim 175, wherein the one or more tolerogenic factors comprise one or more tolerogenic factors selected from the group consisting of PD-L1, HLA-E, HLA-G, CD47, CD200, FASLG, CLC21, MFGE8, SERPIN B9 and any combination thereof.

205. A pharmaceutical composition comprising the engineered cell of claim 175.

206. A method of treating a patient in need of a treatment comprising administering the engineered cell of claim 175 to the patient.

207. A method of generating an engineered cell comprising:
(a) obtaining an isolated cell;
(b) introducing into the cell a CRISPR/Cas nuclease and a guide RNA targeting a blood group antigen gene; and
(c) selecting an engineered cell in which the blood group antigen gene is partially or fully inactivated,
wherein the engineered cell comprises one or more modifications, wherein the one or more modifications:
(i) inactivate or disrupt one or more alleles of:
(1) one or more major histocompatibility class I (MHC-I) molecules and/or one or more molecules that regulate expression of the one or more MHC-I molecules, and/or
(2) one or more MHC class II (MHC-II) molecules and/or one or more molecules that regulate expression of the one or more MHC-II molecules, and/or
(ii) increase expression of one or more tolerogenic factors,
wherein the increased expression of (ii) is relative to an isolated cell that does not comprise the modifications.
